(19) United States
(12) Patent Application Publication
(10) Pub. No.: US 2026/0083853 A1
(43) Pub. Date: Mar. 26, 2026

(54) CD70 ANTIBODY DRUG CONJUGATES AND METHODS OF USING THE SAME

(71) Applicant: Genmab A/S, Valby (DK)

(72) Inventors: Xiao SHANG, Woodinville, WA (US); Haidong LIU, Suzhou, Jiangsu (CN); Julia GAVRILYUK, Woodinville, WA (US); Baiteng ZHAO, Woodinville, WA (US); Tae HAN, Woodinville, WA (US); Zhu CHEN, Woodinville, WA (US)

(21) Appl. No.: 19/123,878

(22) PCT Filed: Oct. 25, 2023
(86) PCT No.: PCT/US2023/077814

§ 371 Date: Apr. 24, 2025

(30) Foreign Application Priority Data
Oct. 26, 2022 (WO) PCT/CN2022/127588
Sep. 14, 2023 (WO) PCT/US2023/074126

(51) Int. Cl. A61K 47/68; A61P 35/00; C07K 16/28

(52) U.S. Cl. CPC A61K 47/68037; A61K 47/68031; A61K 47/6849; A61K 47/6851; A61K 47/6889; A61P 35/00; C07K 16/2875

(57) ABSTRACT

The present invention provides conjugates of CD70 antibodies, and/or antigen binding portions thereof, for use in the treatment of cancer and autoimmune disease.

### CROSS-REFERENCE TO RELATED APPLICATIONS

This application is a 35 U.S.C. § 371 national stage filing of International Application No. PCT/US2023/077814, filed on Oct. 25, 2023, which application claims priority to International Application Nos. PCT/CN2022/127588, filed Oct. 26, 2022, and PCT/CN2023/074126, filed Sep. 14, 2023. The entire contents of the aforementioned applications are incorporated herein by reference.

### SEQUENCE LISTING

The instant application contains a Sequence Listing which has been submitted electronically in XML format via EFS-Web and is hereby incorporated by reference in its entirety. Said XML copy, created on Aug. 2, 2023, is named GMI-409CUS_Sequence-Listing.txt and is 34 KB in size.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must be able to reach its target. If the drug is to be released after antibody internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

An attractive target for cancer therapies employing ADCs is CD70. CD70 is member of the tumor necrosis factor (TNF) family of cell membrane-bound and secreted molecules that are expressed by a variety of normal and malignant cell types. CD70 is a transmembrane type II protein with its carboxyl terminus exposed to the outside of cells and its amino terminus found in the cytosolic side of the plasma membrane (Bowman et al., 1994, J. Immunol. 152:1756-61; Goodwin et al, 1993, Cell 73:447-56). Human CD70 contains a 20 amino acid cytoplasmic domain, an 18 amino acid transmembrane domain, and a 155 amino acid extracellular domain with two potential N-linked glycosylation sites (Bowman et al. supra; Goodwin et al, supra). Based on its homology to TNF-alpha and TNF-beta, a trimeric structure is predicted for CD70 (Petsch et al, 1995, Mol Immunol. 32:761-72).

CD70 has limited expression on normal tissues in humans. This makes CD70 an attractive target for cancer therapies. CD70 expression has been identified on a number of cancers, including renal cell carcer, colon cancer, nasopharyngeal carcinoma, ovarian cancer, pancreatic cancer, certain types of Non-Hodgkin lymphoma and multiple myeloma. Although CD70 is present on a variety of types of cancer, clinical trials with CD70 antibodies and CD70 antibody drug conjugates have met with limited success thus far.

There is a need, therefore, for CD70 antibody drug conjugates generally, and for CD70 antibody drug conjugates in particular that allow for higher drug loading, but that maintain other characteristics of lower loaded conjugates, such as favorable PK properties. Embodiments of the present invention address these and related needs.

### SUMMARY OF THE INVENTION

Provided herein are CD70 antibody drug conjugates (ADCs) and methods of using the same. The CD70 antibody drug conjugates comprise a Binding unit comprising one or more CD70 antibodie(s) or antigen binding portions thereof, a Linker(s), and one or more Drug unit(s). Additionally, provided herein are CD70 ADCs having hydrophilic characteristics that maintain the intrinsic properties of CD70 antibodies conjugated via the Linker(s) to one or more Drug unit(s). In particular, the Linkers aid in maintaining the hydrophilic properties of the CD70 antibodies when conjugated at higher drug loading and/or to hydrophobic drugs and other agents. Also provided are methods of using such conjugates for the treatment of cancer and other diseases. The invention disclosed herein is based in part on CD70 ADCs that specifically bind to CD70 and that exhibit improved properties. CD70 is an important and advantageous therapeutic target for the treatment of certain cancers and autoimmune diseases. The CD70 ADCs provide compositions and methods based on the use of such conjugates in the treatment of CD70+ cancers and other diseases.

In some embodiments, provided is a conjugate comprising a Binding unit bound to one or more Drug units by one or more Linkers, wherein:
(1) the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO:21, SEQ ID NO:22, SEQ ID NO:13, SEQ ID NO:24, SEQ ID NO:25 and SEQ ID NO:26, respectively; SEQ ID NO:21, SEQ ID NO:22, SEQ ID NO:14, SEQ ID NO:24, SEQ ID NO:25 and SEQ ID NO:26, respectively; SEQ ID NO:21, SEQ ID NO:22, SEQ ID NO:15, SEQ ID NO:24, SEQ ID NO:25 and SEQ ID NO:26, respectively; SEQ ID NO:21, SEQ ID NO:22, SEQ ID NO:23, SEQ ID NO:24, SEQ ID NO:25 and SEQ ID NO:18, respectively; and SEQ ID NO:16, SEQ ID NO:17, SEQ ID NO:23, SEQ ID NO:24, SEQ ID NO:25 and SEQ ID NO:26; respectively;
(2) each Linker has the following formula (I):

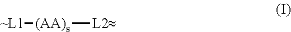

or a salt thereof, wherein:
L1 is a Stretcher unit covalently bound to the Binding unit, wherein the wavy (˜) line indicates an attachment site for the Binding unit;
AA is an Amino Acid unit having from 1 to 12 subunits;
s is 0 or 1;
L2 is a Linker Subunit having from 1 to 4 attachment sites for the Drug unit, wherein the double wavy (≈) line indicates an attachment site for the Drug Unit; and wherein at least one Polar unit is present within the Amino Acid unit, the Linker Subunit, the Stretcher unit, or combinations thereof, and wherein the Polar unit(s) is selected from Sugar units, PEG units, Carboxyl units, and combinations thereof; and
(3) each Drug unit is covalently attached to the Linker Subunit at (≈).

In some embodiments, provided is a conjugate wherein the VH and VL regions of the Binding unit have amino acid sequences that are selected from the pairs of amino acid sequences set forth in the group consisting of: SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; and SEQ ID NO:11 and SEQ ID NO:12; respectively. In some embodiments, provided is a conjugate wherein the VH and VL regions of the Binding unit have amino acid sequences that are selected from the pairs of amino acid sequences set forth in the group consisting of: SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; and SEQ ID NO:11 and SEQ ID NO:12; respectively, wherein the heavy and light chain framework regions are optionally modified with from 1 to 8 amino acid substitutions, deletions or insertions in the framework regions. In some embodiments, a conjugate is provided wherein HCDR1, HCDR2 and HCDR3 and LCDR1, LCDR2 and LCDR3 of the Binding unit have the amino acid sequences set forth in SEQ ID NO:21, SEQ ID NO:22, and SEQ ID NO:15, and SEQ ID NO:24, SEQ ID NO:25 and SEQ ID NO:26, respectively.

In some embodiments, provided is a conjugate wherein the framework regions of the Binding unit are human framework regions. In some embodiments, is provided a conjugate wherein the Binding unit is an antibody or an antigen-binding portion thereof. In some embodiments, the Binding unit is a monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, provided is a conjugate wherein the Binding unit has a heavy chain variable region further comprising a heavy chain constant region. In some embodiments, provided is a conjugate wherein the heavy chain constant region of the Binding unit is of the IgG isotype. In some embodiments, provided is a conjugate wherein the heavy chain constant region of the Binding unit is an IgG1 constant region. In some embodiments, provided is a conjugate wherein the heavy chain constant region of the Binding unit is an IgG4 constant region. In some embodiments, provided is a conjugate wherein the IgG1 constant region of the Binding unit has the amino acid sequence set forth in SEQ ID NO:28. In some embodiments, provided is a conjugate wherein the Binding unit has a light chain variable region further comprising a light chain constant region. In some embodiments, provided is a conjugate wherein the light chain constant region of the Binding unit is of the kappa isotype. In some embodiments, provided is a conjugate wherein the light chain constant region of the Binding unit has the amino acid sequence set forth in SEQ ID NO:29. In some embodiments, provided is a conjugate wherein the heavy chain constant region of the Binding unit further comprises at least one amino acid modification that decreases binding affinity to a human Fc receptor (such as FcgammaRIII). In some embodiments, provided is a conjugate wherein the heavy chain constant region of the Binding unit further comprises at least one amino acid modification that decreases binding affinity to human FcgammaRIII.

In some embodiments, provided is a conjugate wherein the Binding unit is mono-specific. In some embodiments, provided is a conjugate wherein the Binding unit is bivalent. In some embodiments, provided is a conjugate wherein the Binding unit is bispecific.

In some embodiments, provided is a pharmaceutical composition comprising any of the conjugates as described herein and a pharmaceutically acceptable carrier.

In some embodiments, provided is a conjugate wherein the Sugar unit of the Linker has the following formula:

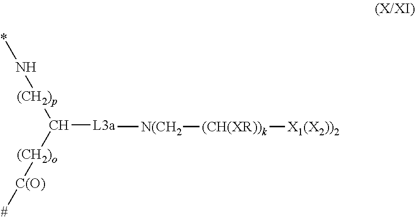

or a salt thereof, wherein:
   each X is independently selected from NH or 0;
   each R is independently selected from hydrogen, acetyl, a monosaccharide, a disaccharide, and a polysaccharide;
   each X₁ is independently selected from CH₂ and C(O);
   each X₂ is independently selected from H, OH and OR;
   k is 1 to 10;
   L3a is selected from C₁-C₁₀ alkylene and polyethylene glycol having from 1 to 24 ethylene glycol subunits;
   p and o are independently 0 to 2; and
   each * and each # indicate an attachment site for another subunit of an Amino Acid unit (AA), a Linker subunit L2, or a Stretcher unit (L1).

In some embodiments, provided is a conjugate wherein the PEG unit of the Linker has a formula selected from:
(a)

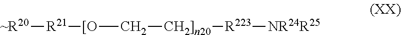

or a salt thereof, wherein:
   R²⁰ is a functional group for attachment to a subunit of the Amino Acid unit, a Stretcher unit and/or a portion of the Linker Subunit L2;
   R²¹ and R²² are each, independently, optional C₁-C₃ alkylene;
   R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle; provided that both R²⁴ and R²⁵ are not H;
   the wavy line (˜) indicates the attachment site to R²⁰; and
   n20 is 1 to 26;
   or
(b)

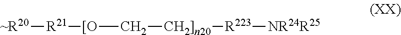

or a salt thereof, wherein:
   R²⁰ is a functional group for attachment to a subunit of the Amino Acid unit, a Stretcher unit and/or a portion of the Linker Subunit L2;
   R²¹ and R²² are each, independently, optional C₁-C₃ alkylene;
   one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is a polyethylene glycol, optionally having 1 to 24 ethylene glycol subunits;
   the wavy line (˜) indicates the attachment site to R²⁰; and
   n20 is 1 to 26;
   or
(c)

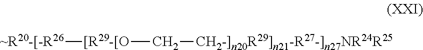

or a salt thereof, wherein:
   R²⁰ is a functional group for attachment to a subunit of an Amino Acid unit, a Stretcher unit and/or a portion of a Linker Subunit L2;
   R²⁶ and R²⁷ are each optional and are, independently, selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
   one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle; provided that both R²⁴ and R²⁵ are not H;
   each R²⁹ is optional and independently selected from —C(O)—, —NH—, —C(O)—C₁-C₆ alkenylene-, —NH—C₁-C₆ alkenylene-, —C₁-C₆ alkenylene-NH—, —C₁-C₆ alkenylene-C(O)—, —NH(CO)NH—, and triazole;
   the wavy line (˜) indicates the attachment site to R²⁰;
   n20 is 1 to 26;
   n21 is 1 to 4; and
   n27 is 1 to 4.

In some embodiments, provided is a conjugate wherein R²⁴ and R²⁵ are each independently selected from H and polyhydroxyl group, provided that R²⁴ and R²⁵ are not both H.

In some embodiments, provided is a conjugate wherein the polyhydroxyl group is a linear monosaccharide, optionally selected from a C6 or C5 sugar, sugar acid or amino sugar.

In some embodiments, provided is a conjugate wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

In some embodiments, provided is a conjugate wherein one of R²⁴ and R²⁵ is a linear monosaccharide and the other is a cyclic monosaccharide.

In some embodiments, provided is a conjugate wherein R²⁴ and R²⁵ are independently selected from cyclic monosaccharides, disaccharides and polysaccharides.

In some embodiments, provided is a conjugate wherein R²⁴ and R²⁵ are independently selected from a linear monosaccharide and a substituted linear monosaccharide, wherein the substituted linear monosaccharide is substituted with a monosaccharide, a disaccharide or a polysaccharide.

In some embodiments, provided is a conjugate wherein R²⁴ and R²⁵ are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide.

In some embodiments, provided is a conjugate wherein one of R²⁴ and R²⁵ is a —C(O)— polyhydroxyl group or substituted —C(O)-polyhydroxyl group, and the other of R²⁴ and R²⁵ is a H, —C(O)— polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O-alkyl, aryl, carboxyl, ester, or amide.

In some embodiments, provided is a conjugate wherein R²⁴ and R²⁵ are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl and —C₁-C₃ alkyl are substituted with hydroxyl and/or carboxyl; provided that both R²⁴ and R²⁵ are not H.

In some embodiments, provided is a conjugate wherein one of R²⁴ and R²⁵ is selected from H, substituted —C(O)—C₁-C₈ alkyl, substituted —C(O)—C₁-C₄ alkyl, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl; provided that both R²⁴ and R²⁵ are not H.

In some embodiments, provided is a conjugate wherein each monosaccharide is independently selected from:
a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine;
a sugar acid selected from gluconic acid, aldonic acid, uronic acid and ulosonic acid; or
an amino sugar is selected from glucosamine, N-acetyl glucosamine, galactosamine, and N-acetyl galactosamine.

In some embodiments, provided is a conjugate wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, provided is a conjugate wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, provided is a conjugate wherein the PEG unit has a formula selected from:

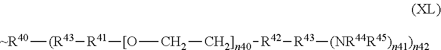

or a salt thereof, wherein:
   R⁴⁰ is a functional group for attachment to a subunit of the Amino Acid unit, the Stretcher unit and/or a portion of the Linker Subunit L2;
   R⁴¹ and R⁴² are absent or are each, independently, C₁-C₆ alkylene;
   each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
   R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
   provided that both R⁴⁴ and R⁴⁵ are not H;
   the wavy line (˜) indicates the attachment site to R⁴⁰;
   n40 is 1 to 26;
   n41 is 1 to 6; and
   n42 is 1 to 6.

In some embodiments, provided is a conjugate wherein the PEG unit has a formula selected from:

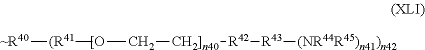

or a salt thereof, wherein:
   R⁴⁰ is a functional group for attachment to a subunit of the Amino Acid unit, the Stretcher unit and/or a portion of the Linker Subunit L2;
   R⁴¹ and R⁴² are absent or are each, independently, C₁-C₆ alkylene;
   R⁴³ is absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
   R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
   provided that both R⁴⁴ and R⁴⁵ are not H;
   the wavy line (˜) indicates the attachment site to R⁴⁰;
   n40 is 1 to 26;
   n41 is 1 to 6; and
   n42 is 1 to 6.

In some embodiments, provided is a conjugate wherein the PEG unit has a formula selected from:

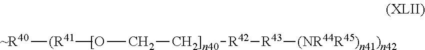

or a salt thereof, wherein:
   R⁴⁰ is a functional group for attachment to a subunit of the Amino Acid unit, the Stretcher unit and/or a portion of the Linker Subunit L2;
   R⁴¹ and R⁴² are absent or are each, independently, C₁-C₃ alkylene;
   R⁴³ is absent or is selected from selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
   R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
   provided that both R⁴⁴ and R⁴⁵ are not H;
   the wavy line (˜) indicates the attachment site to R⁴⁰.
   n40 is 1 to 26;
   n41 is 1 to 4; and
   n42 is 1 to 4.

In some embodiments, provided is a conjugate wherein R⁴⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, provided is a conjugate wherein R²⁰ or R⁴⁰ has one of the following structures:

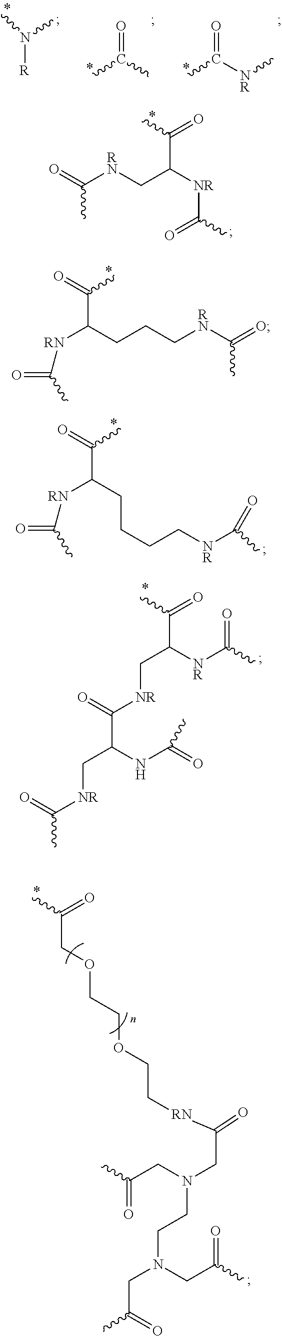

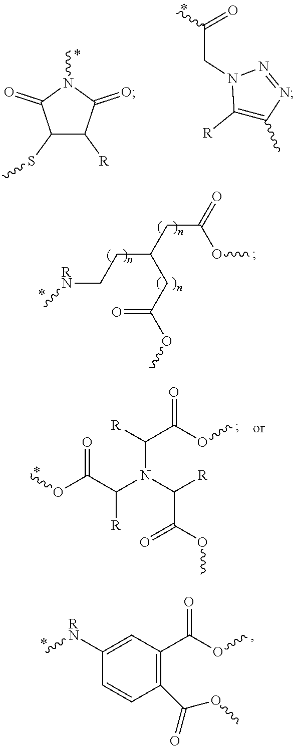

wherein R═H or C₁₋₆alkyl; and
n=0 to 12
or a stereoisomer thereof, wherein the (*) indicates the attachment site of R²⁰ or R⁴⁰ to a subunit of the Amino Acid unit, the Stretcher unit and/or a portion of the Linker Subunit L2 and the ( [symbol] ) indicates the attachment site of R²⁰ or R⁴⁰ to the remainder of the PEG unit.

In some embodiments, provided is a conjugate wherein R²⁰ or R⁴⁰ has one of the following structures:

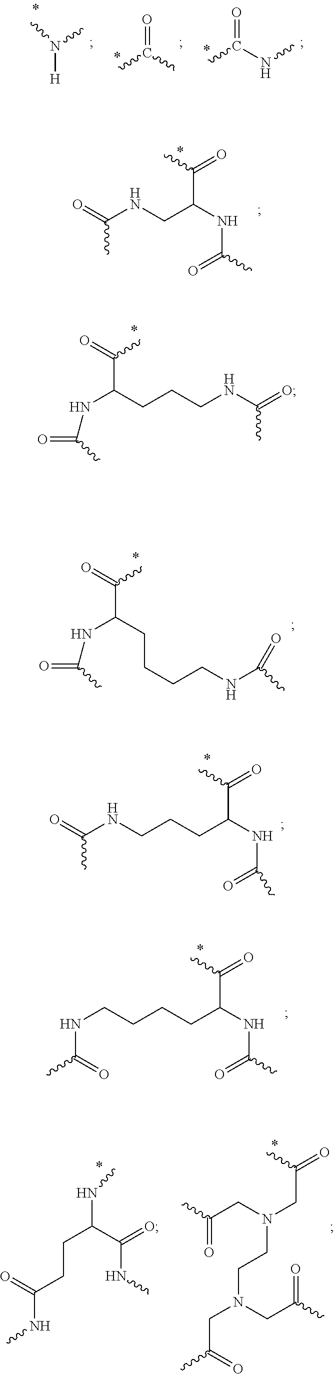

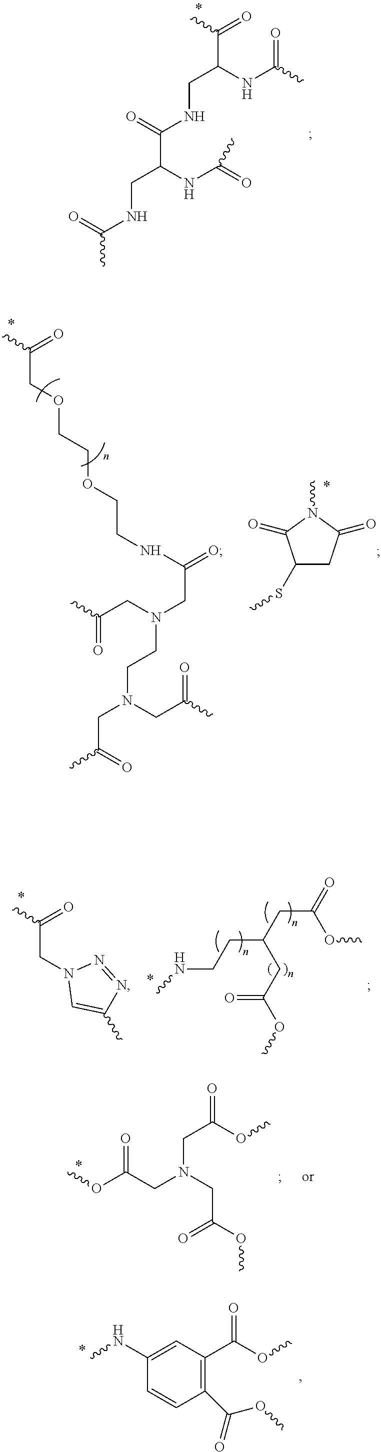

wherein n=0 to 12
or a stereoisomer thereof, wherein the (*) indicates the attachment site of R²⁰ or R⁴⁰ to a subunit of the Amino Acid unit, the Stretcher unit and/or a portion of the Linker Subunit L2 and the ( [symbol] ) indicates the attachment site of R²⁰ or R⁴⁰ to the remainder of the PEG unit.

In some embodiments, provided is a conjugate wherein R⁴³—(NR⁴⁴R⁴⁵)ₙ₄₁, when NR⁴³ is present, has one of the following structures:

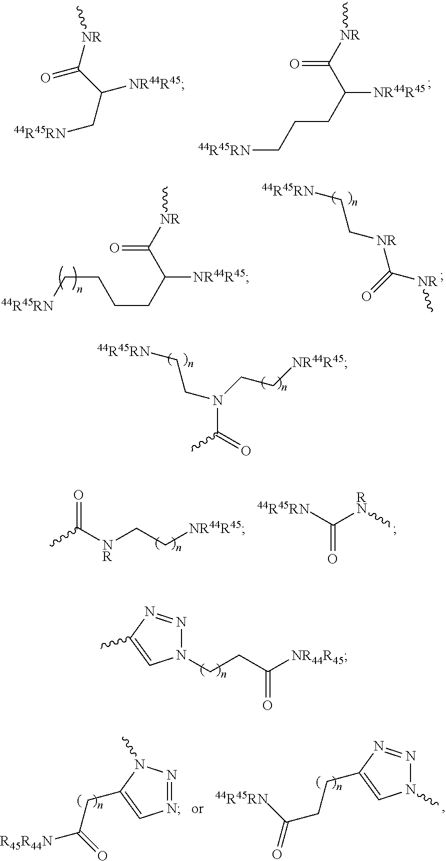

wherein R═H, C₁₋₆alkyl, polyhydroxyl, or substituted polyhydroxyl
or a stereoisomer thereof, wherein the ( [symbol] ) indicates the attachment site of R⁴³ to the remainder of the PEG unit.

In some embodiments, provided is a conjugate wherein R⁴³—(NR⁴⁴R⁴⁵)ₙ₄₁, when NR⁴³ is present, has one of the following structures:

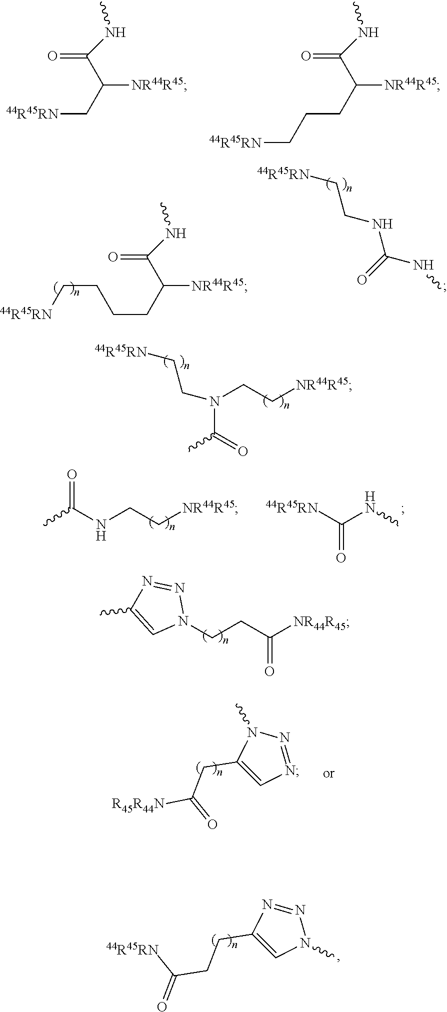

or a stereoisomer thereof, wherein the ( [symbol] ) indicates the attachment site of R⁴³ to the remainder of the PEG unit.

In some embodiments, provided is a conjugate wherein —NR⁴⁴R⁴⁵ has one of the following structures:

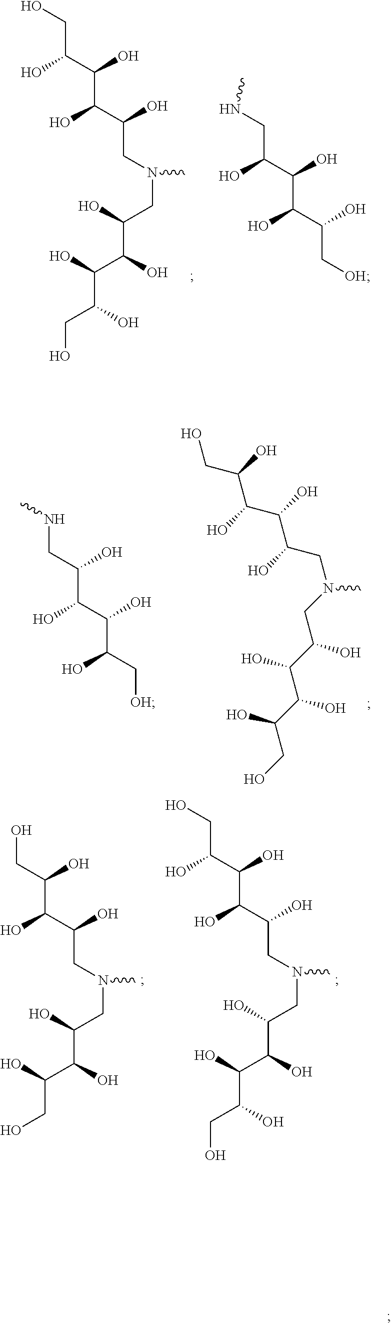

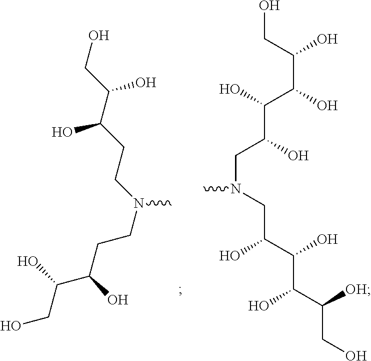

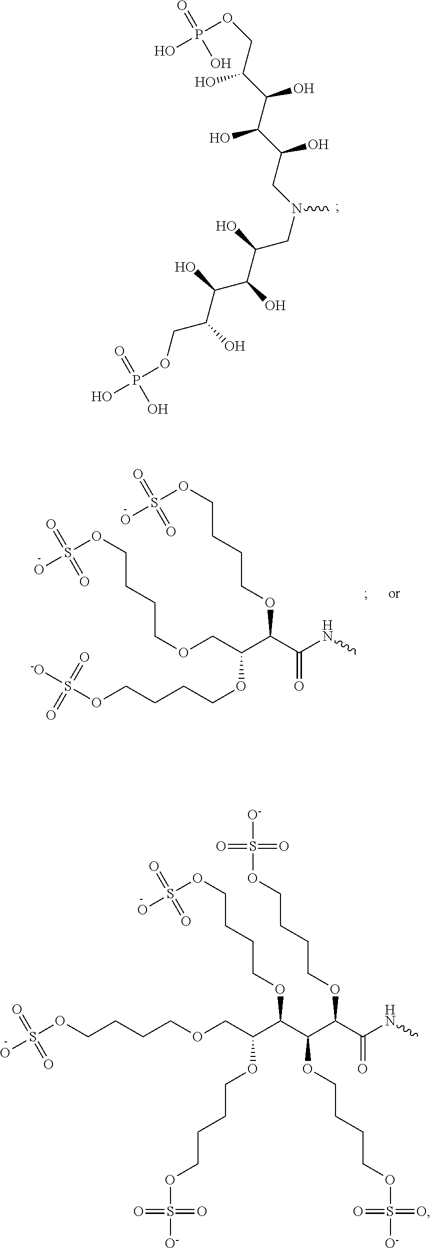

or a stereoisomer thereof, wherein the ( [symbol] ) indicates the attachment site of —NR⁴⁴R⁴⁵ to the remainder of the PEG unit.

In some embodiments, provided is a conjugate comprising a PEG unit having a formula selected from:

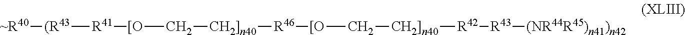

or a salt thereof, wherein:
   R⁴⁰ is a functional group for attachment to a subunit of the Amino Acid unit, the Stretcher unit and/or a portion of the Linker Subunit L2;
   R⁴¹ and R⁴² are absent or are each, independently, C₁-C₆ alkylene;
   each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
   R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; provided that both R⁴⁴ and R⁴⁵ are not H;
   R⁴⁶ is selected from amino, amino-alkyl-amino, or —NH—C(O)—NH—S(O)₂—NH—; the wavy line (˜) indicates the attachment site to R⁴⁰.
   n40 is 1 to 26;
   n41 is 1 to 6; and
   n42 is 1 to 6.

In some embodiments, provided is a conjugate comprising a PEG unit having a formula selected from:

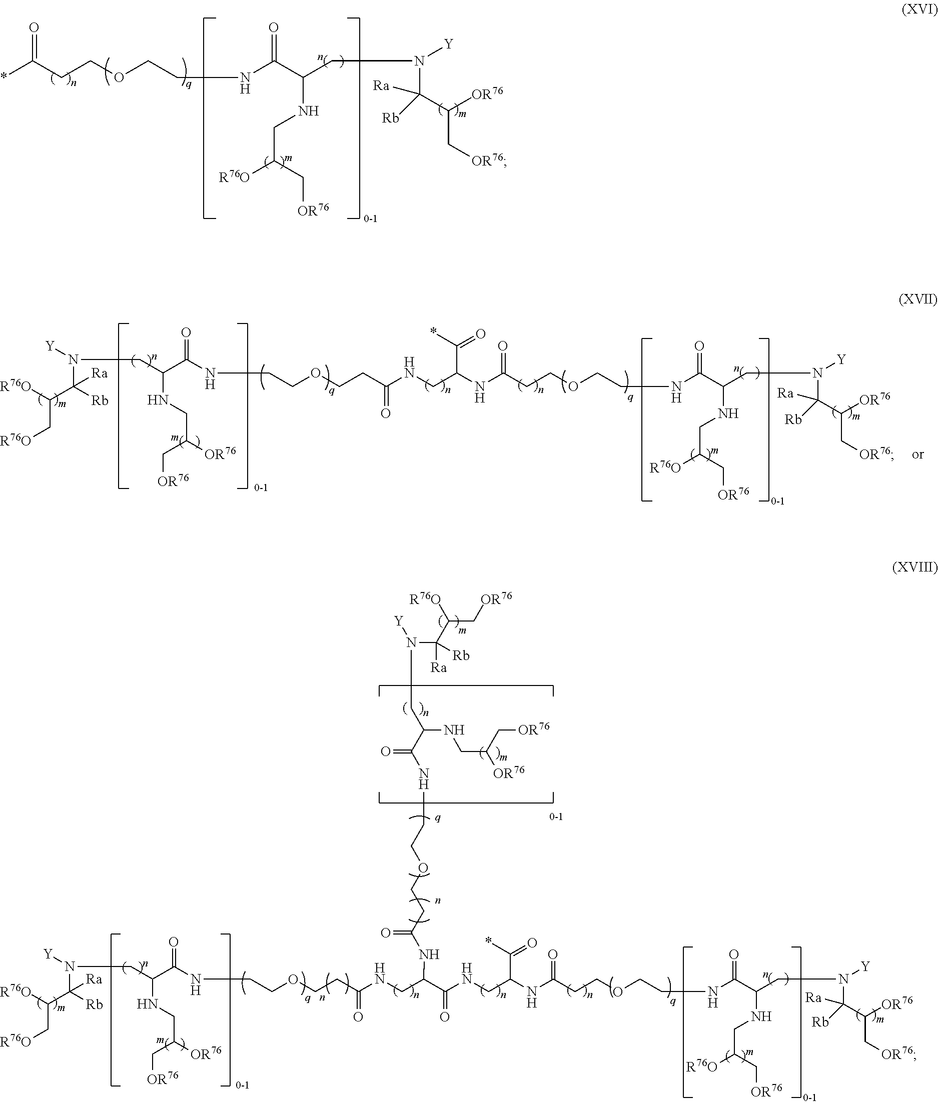

or a stereoisomer or salt thereof, wherein:
   each Y is independently R⁷⁶ or

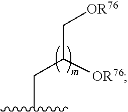

each R⁷⁶ is independently H, acetyl, —P(═O)(OH)₂, or —(CH₂)ᵥO—S(═O)₂(OH);
each Rₐ and R_{b} is independently H or Rₐ and R_{b} are taken together with the carbon to which they are attached to form an oxo group;
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

In some embodiments, provided is a conjugate wherein the PEG unit has a formula selected from:

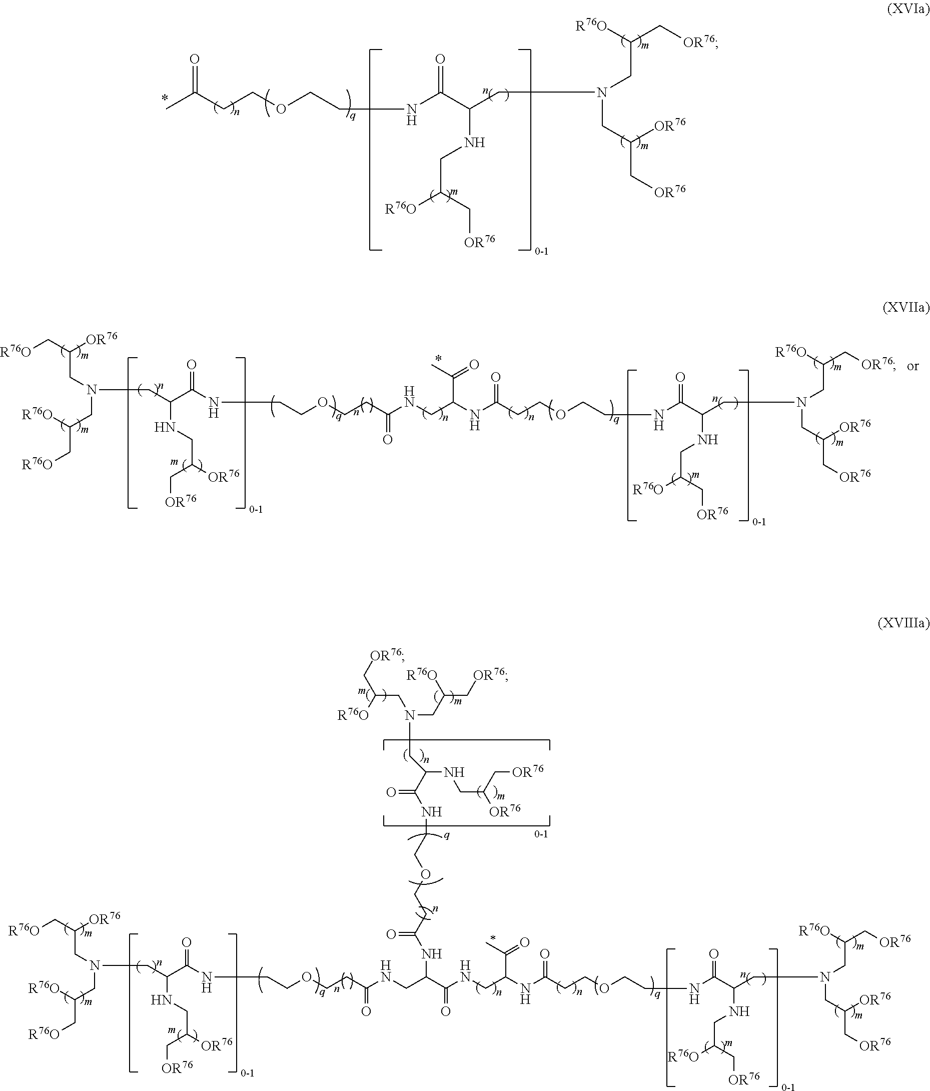

or a stereoisomer or salt thereof, wherein:
   each R⁷⁶ is independently H, acetyl, —P(═O)(OH)₂, or —(CH₂)ᵥS(═O)₂(OH);
   each q is independently 1-26;
   each m is independently 1 to 4;
   each n is independently 1 to 4;
   each v is independently 1 to 6; and
   each * indicates an attachment site for a subunit of the Amino Acid unit (AA), the Linker subunit L2, or the Stretcher unit (L1).

In some embodiments, provided is a conjugate wherein the PEG unit has a formula selected from:

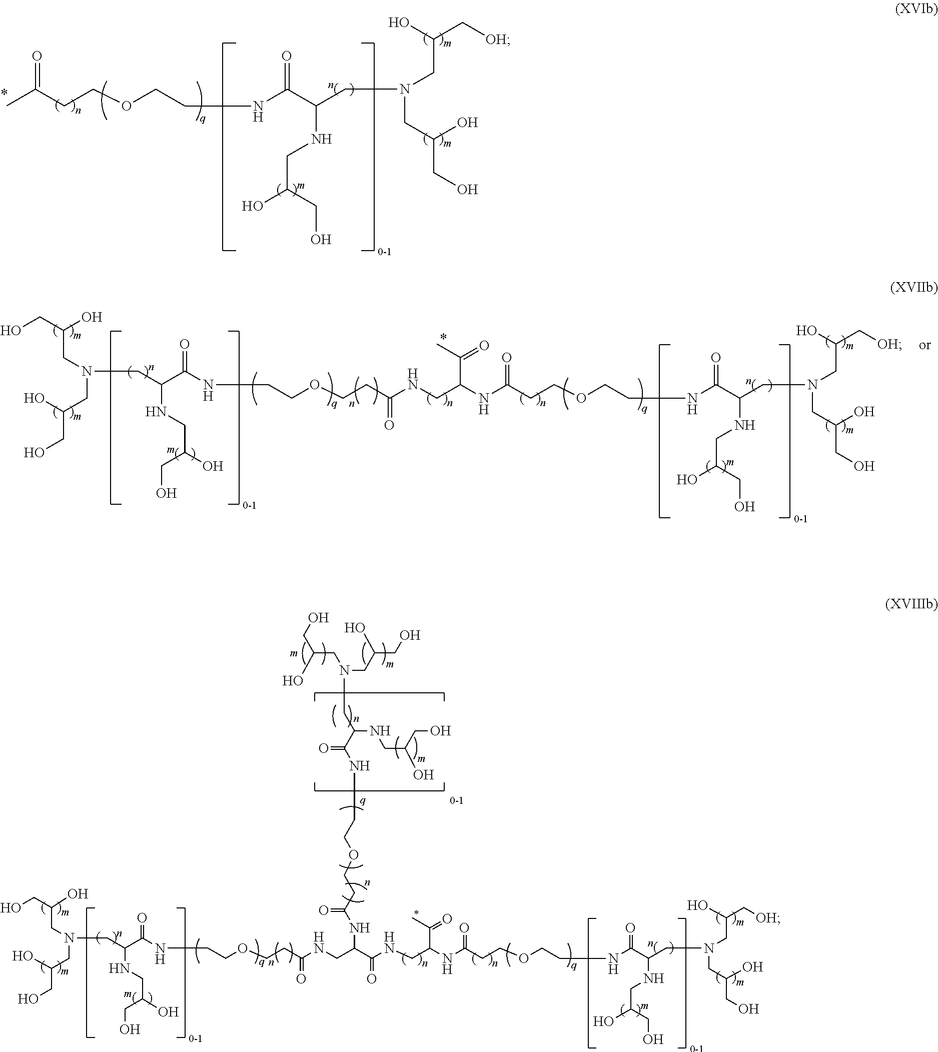

or a stereoisomer or salt thereof, wherein:
   each q is independently 1-26;
   each m is independently 1 to 4;
   each n is independently 1 to 4; and
   each * indicates an attachment site for a subunit of the Amino Acid unit (AA), the Linker subunit L2, or the Stretcher unit (L1).

In some embodiments, provided is a conjugate wherein Y is R⁷⁶.

In some embodiments, provided is a conjugate wherein Y is

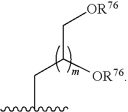

In some embodiments, provided is a conjugate wherein each Rₐ and R_{b} is independently H.

In some embodiments, provided is a conjugate wherein Rₐ and R_{b} are taken together with the carbon to which they are attached to form an oxo group.

In some embodiments, provided is a conjugate wherein q is 10-20.

In some embodiments, provided is a conjugate wherein q is 12.

In some embodiments, provided is a conjugate comprising a Carboxyl unit having the following formula:

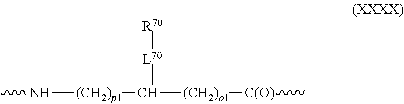

or a salt thereof, wherein:
   (a)
      L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is ˜NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is absent or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
      each wavy line (˜) indicates an attachment site for another subunit of an Amino Acid unit (AA), the Linker subunit L2, or the Stretcher unit (L1); and
      each of p1 and o1 are independently selected from 0 to 2;
      or
   (b)
      L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is ˜NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
      each wavy line (˜) indicates an attachment site for another subunit of an Amino Acid unit (AA), the Linker subunit L2, or the Stretcher unit (L1); and
      each of p1 and o1 are independently selected from 0 to 2;
      or
   (c)
      L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is ˜N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
      each wavy line (˜) indicates an attachment site for another subunit of an Amino Acid unit (AA), the Linker subunit L2, or the Stretcher unit (L1); and
      each of p1 and o1 are independently selected from 0 to 2.

In some embodiments, provided is a conjugate comprising at least one Sugar unit.

In some embodiments, provided is a conjugate comprising at least one PEG unit.

In some embodiments, provided is a conjugate comprising at least one Carboxyl unit.

In some embodiments, provided is a conjugate comprising at least two Polar units, each Polar unit selected from a Sugar unit, a PEG unit and a Carboxyl unit.

In some embodiments, provided is a conjugate comprising at least one Sugar unit and a PEG unit or a Carboxyl unit.

In some embodiments, provided is a conjugate comprising at least one Carboxyl unit and a PEG unit.

In some embodiments, provided is a conjugate wherein the Amino Acid unit (AA) is present (s=1).

In some embodiments, provided is a conjugate wherein the Amino Acid unit comprises at least one Polar unit.

In some embodiments, provided is a conjugate wherein L2 or AA-L2 has one of the following structures, or a stereoisomer thereof:

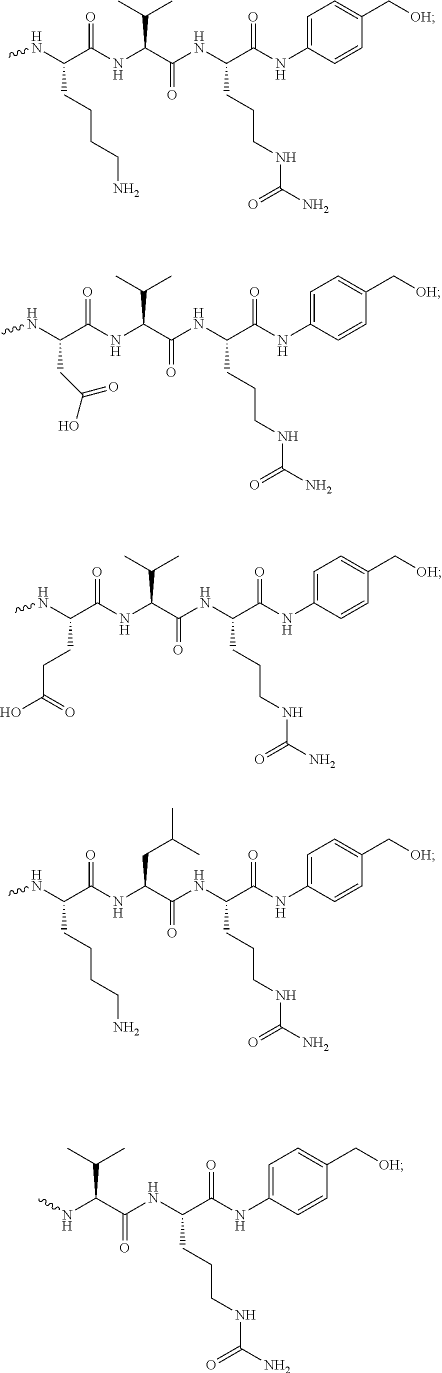

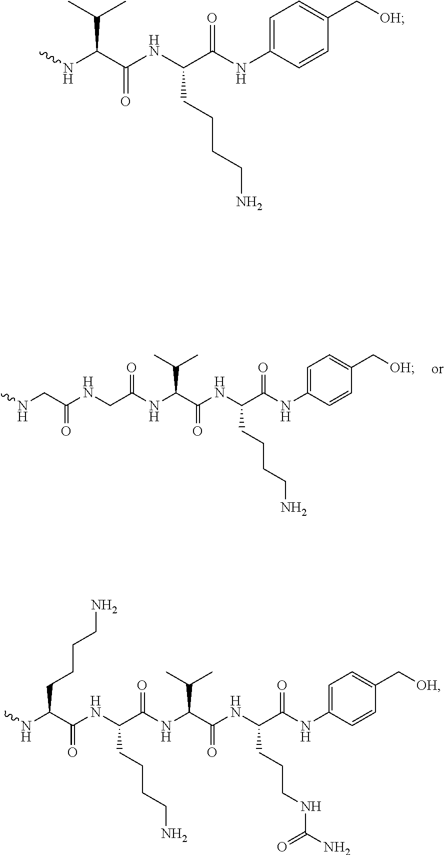

wherein the wavy line on the amino group indicates an attachment site for a Stretcher unit or an Amino Acid unit, and the Drug unit is attached to the benzyl alcohol.

In some embodiments, provided is a conjugate wherein the Linker comprises ˜AA-L2˜ having a formula selected from the following:

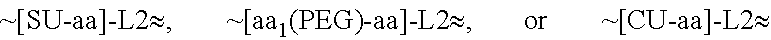

wherein the square brackets indicate the Amino Acid unit, each aa is an optional subunit of AA, L2 is the Linker Subunit, each wavy line (˜) indicates an attachment site for a Stretcher unit; aa₁(PEG) is a PEG unit attached to an amino acid subunit of AA, SU is a Sugar unit attached to a subunit of AA or to L2, and CU is a Carboxyl unit attached to a subunit of AA or to L2; and the double wavy (≈) line indicates an attachment site for a Drug unit, wherein aa and aa₁ are independently selected from alpha, beta and gamma amino acids and derivatives thereof.

In some embodiments, provided is a conjugate wherein the Linker comprises ˜AA-L2˜ having a formula selected from the following:

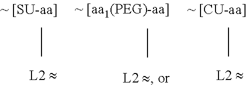

wherein the square brackets indicate the Amino Acid unit, each aa is an amino acid subunit of AA, L2 is the Linker Subunit attached to a side chain of aa, the wavy line (˜) indicates an attachment site for a Stretcher unit; aa₁(PEG) is a PEG unit attached to aa, SU is a Sugar unit attached to aa, CU is a Carboxyl unit attached to aa, and the double wavy (≈) line indicates an attachment site for a Drug unit; wherein aa and aa₁ are independently selected from alpha, beta and gamma amino acids and derivatives thereof.

In some embodiments, provided is a conjugate wherein the Amino Acid unit comprises at least two Polar units.

In some embodiments, provided is a conjugate wherein the Linker comprises ˜AA-L2˜ having a formula selected from the following:

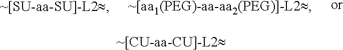

wherein the square brackets indicate the Amino Acid unit, aa is an optional subunit of AA, L2 is the Linker Subunit, the wavy line (˜) indicates an attachment site for a Stretcher unit; each of aa₁(PEG) and aa₂(PEG) is a PEG unit attached to aa or to the other PEG unit; each SU is a Sugar unit attached to aa or the other Sugar unit, each CU is a Carboxyl unit attached to aa or to the other Carboxyl unit, and the double wavy (≈) line indicates an attachment site for a Drug unit; wherein aa, aa₁ and aa₂ are independently selected from selected from alpha, beta and gamma amino acids and derivatives thereof.

In some embodiments, provided is a conjugate wherein the Linker comprises ˜AA-L2˜ having a formula selected from the following:

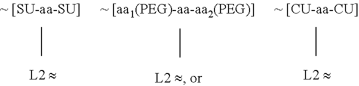

wherein the square brackets indicate the Amino Acid unit, aa is an amino acid subunit of AA, L2 is a Linker Subunit attached to a side chain of aa, each wavy line (˜) indicates an attachment site for a Stretcher unit; each of aa₁(PEG) and aa₂(PEG) is a PEG unit attached to aa, each SU is a Sugar unit attached to aa; each CU is a Carboxyl unit attached to aa; and the double wavy (≈) line indicates an attachment site for a Drug unit; wherein each of aa, aa₁ and aa₂ is independently selected from alpha, beta and gamma amino acids and derivatives thereof.

In some embodiments, provided is a conjugate wherein Linker Subunit L2 is a cleavable linker unit.

In some embodiments, provided is a conjugate wherein Linker Subunit L2 comprises a peptide that is cleavable by an intracellular protease.

In some embodiments, provided is a conjugate wherein the cleavable peptide comprises a valine-citrulline peptide, a valine-alanine peptide, a valine-lysine peptide, a phenylalanine-lysine peptide, or a glycine-glycine-phenylalanine-glycine peptide.

In some embodiments, provided is a conjugate wherein Linker Subunit L2 comprises at least one Polar unit.

In some embodiments, provided is a conjugate wherein the Polar unit is a Sugar unit (SU).

In some embodiments, provided is a conjugate wherein the cleavable peptide comprises a SU-valine-citrulline peptide, a SU-valine-lysine peptide, a SU-valine-alanine peptide, a SU-phenylalanine-lysine peptide, or a SU-glycine-glycine-phenylalanine-glycine peptide.

In some embodiments, provided is a conjugate wherein the Polar unit is a Carboxyl unit (CU).

In some embodiments, provided is a conjugate wherein the cleavable peptide comprises a CU-valine-citrulline peptide, a CU-valine-lysine peptide, a valine-(CU-lysine) peptide, a CU-valine-alanine peptide, a CU-phenylalanine-lysine peptide, a phenylalanine-(CU-lysine) peptide or a CU-glycine-glycine-phenylalanine-glycine peptide, wherein CU-lysine is a Carboxyl unit comprising a lysine residue.

In some embodiments, provided is a conjugate wherein the Polar unit is a PEG unit (PEG).

In some embodiments, provided is a conjugate wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG)) peptide or a Lys(PEG)-glycine-glycine-phenylalanine-glycine peptide, wherein Lys(PEG) and Cit(PEG) comprise a PEG unit attached to a lysine residue or a citrulline residue, respectively.

In some embodiments, provided is a conjugate wherein the cleavable peptide is attached to a para-aminobenzyl alcohol self immolative group (PABA).

In some embodiments, provided is a conjugate wherein L2 is attached to a side chain of a subunit of AA.

In some embodiments, provided is a conjugate wherein the Amino Acid unit is joined to Linker Subunit L2 by a non-peptidic linking group.

In some embodiments, provided is a conjugate wherein the non-peptidic linking group is selected from C₁-C₁₀ alkylene, C₂-C₁₀ alkenylene, C₂-C₁₀ alkynylene, or polyethylene glycol.

In some embodiments, provided is a conjugate wherein the Linker further comprises a Stretcher unit.

In some embodiments, provided is a conjugate wherein the Stretcher unit is selected from the following:

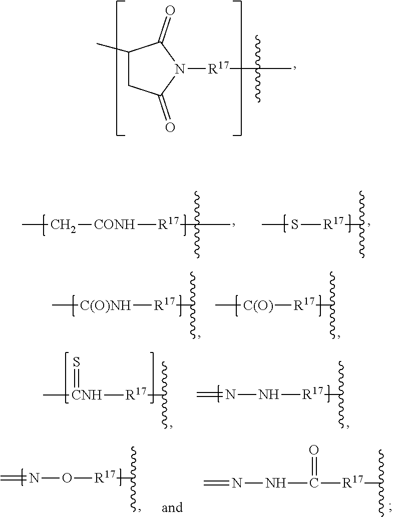

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or wherein the Stretcher unit comprises maleimido(C₁-C₁₀alkylene-C(O)—, maleimido(CH₂OCH₂)ₚ₂(C₁-C₁₀alkylene)C(O)—, maleimido(C₁-C₁₀alkylene)(CH₂OCH₂)ₚ₂C(O)—, or a ring open form thereof, wherein p2 is from 1 to 26.

In some embodiments, provided is a conjugate wherein the Stretcher unit is selected from the following:

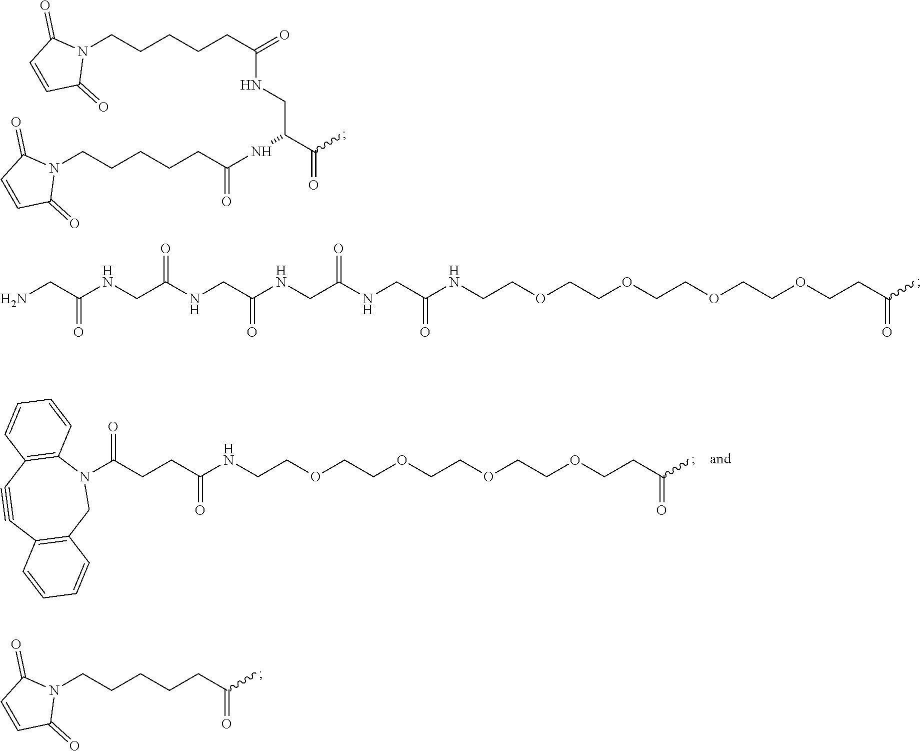

wherein the wavy line [symbol] indicates an attachment site of the Stretcher unit to an Amino Acid unit or to a Linker Subunit L2, and the attachment site to the Binding unit is on a maleimide, primary amine or alkyne functional group.

In some embodiments, provided is a conjugate comprising any of the Binding units described herein, at least one Linker attached to the Binding unit; and at least one Drug unit attached to each Linker. In some embodiments, provided is a conjugate wherein each Drug unit is selected from a cytotoxic agent, an immune modulatory agent, a nucleic acid, a growth inhibitory agent, a PROTAC, a toxin, a radioactive isotope, and a chelating ligand. In some embodiments, provided is a conjugate wherein each Linker is attached to the Binding unit via an interchain disulfide residue, a lysine residue, an engineered cysteine residue, a glycan, a modified glycan, an N-terminal residue of the Binding unit or a polyhistidine residue attached to the Binding unit. In some embodiments, the average drug loading of the conjugate is from about 1 to about 8, about 2, about 4, about 6, about 8, about 10, about 12, about 14, about 16, about 3 to about 5, about 6 to about 8, or about 8 to about 16. In some embodiments, the average drug loading of the conjugate is about 1, about 2, about 4, about 6, about 8, about 10, about 12, about 14, about 16. In some embodiments, the average drug loading of the conjugate is from about 1 to about 8, about 2 to about 8, about 4 to about 8. In some embodiments, the average drug loading of the conjugate is from about 1 to about 12, about 2 to about 12, about 4 to about 12, about 6 to about 12, about 8 to about 12, In some embodiments, the average drug loading of the conjugate is from about 1 to about 16, about 2 to about 16, about 4 to about 16, about 6 to about 16, about 8 to about 16, about 10 to about 16, about 12 to about 16.

In some embodiments, the average drug-to-antibody ratio (DAR) is from about 1 to about 8, about 2, about 4, about 6, about 8, about 10, about 12, about 14, about 16, about 3 to about 5, about 6 to about 8, or about 8 to about 16. In some embodiments, the DAR is about 1, about 2, about 4, about 6, about 8, about 10, about 12, about 14, about 16. In some embodiments, the DAR is from about 1 to about 8, about 2 to about 8, about 4 to about 8. In some embodiments, the DAR is from about 1 to about 12, about 2 to about 12, about 4 to about 12, about 6 to about 12, about 8 to about 12, In some embodiments, the DAR is from about 1 to about 16, about 2 to about 16, about 4 to about 16, about 6 to about 16, about 8 to about 16, about 10 to about 16, about 12 to about 16.

In some embodiments, provided is a conjugate wherein the Drug Unit is a cytotoxic agent. In some embodiments, provided is a conjugate wherein the cytotoxic agent is selected from the group consisting of an auristatin, a maytansinoid, a camptothecin, a duocarmycin, or a calicheamicin. In some embodiments, provided is a conjugate wherein the cytotoxic agent is an auristatin. In some embodiments, provided is a conjugate wherein the cytotoxic agent is MMAE or MMAF. In some embodiments, provided is a conjugate wherein the cytotoxic agent is a camptothecin. In some embodiments, provided is a conjugate wherein the cytotoxic agent is exatecan. In some embodiments, provided is a conjugate wherein the cytotoxic agent is a diastereomer of exatecan. In some embodiments, provided is a conjugate wherein the cytotoxic agent is SN-38. In some embodiments, provided is a conjugate wherein the cytotoxic agent is a calicheamicin. In some embodiments, provided is a conjugate wherein the cytotoxic agent is a maytansinoid. In some embodiments, provided is a conjugate wherein the maytansinoid is maytansine, maytansinol or a maytansine analog in DM1, DM3 and DM4, and ansamatocin-2.

In some embodiments, provided is a conjugate wherein the Linker is a cleavable linker. In some embodiments, provided is a conjugate wherein the Linker comprises mc-VC-PAB, CL2, CL2A or (Succinimid-3-yl-N)—(CH2)n-C(═O)-Gly-Gly-Phe-Gly-NH—CH2-O—CH2-(C═O)—(SEQ ID NO: 34), wherein n=1 to 5. In some embodiments, provided is a conjugate wherein the Linker comprises me-VC-PAB. In some embodiments, provided is a conjugate wherein the Linker comprises CL2A. In some embodiments, provided is a conjugate wherein the Linker comprises CL2. In some embodiments, provided is a conjugate wherein the Linker comprises (Succinimid-3-yl-N)—(CH2)n-C(═O)-Gly-Gly-Phe-Gly-NH—CH2-O—CH2-(C═O)—(SEQ ID NO: 34). In some embodiments, provided is a conjugate wherein the Linker is attached to at least one molecule of exatecan.

In some embodiments, provided is a conjugate wherein the Drug unit is an immune modulatory agent. In some embodiments, provided is a conjugate wherein the immune modulatory agent is selected from the group consisting of a TRL7 agonist, a TLR8 agonist, a STING agonist, or a RIG-I agonist. In some embodiments, provided is a conjugate wherein the immune modulatory agent is an TLR7 agonist. In some embodiments, provided is a conjugate wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, provided is a conjugate wherein the immune modulatory agent is a TLR8 agonist. In some embodiments, provided is a conjugate wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, provided is a conjugate wherein the immune modulatory agent is a STING agonist. In some embodiments, provided is a conjugate wherein the immune modulatory agent is a RIG-I agonist. In some embodiments, provided is a conjugate wherein the RIG-I agonist is selected from KIN1148, SB-9200, KIN700, KIN600, KIN500, KIN100, KIN101, KIN400 and KIN2000.

In some embodiments, provided is a conjugate wherein the Drug unit is a chelating ligand. In some embodiments, provided is a conjugate wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

In some embodiments, provided is a pharmaceutical composition comprising any of the conjugates described herein and a pharmaceutically acceptable carrier.

In some embodiments, provided is method of treating a CD70+ cancer, comprising administering to a subject in need thereof a therapeutically effective amount of any of the conjugates described herein or any of the pharmaceutical compositions described herein. In some embodiments, the CD70+ cancer is a solid tumor or a hematologic malignancy. In some embodiments, the CD70+ cancer is selected from hepatocellular cancer, colorectal cancer, pancreatic cancer, ovarian cancer, indolent Non-Hodgkin's lymphoma, Non-Hodgkin's lymphoma, cancers of the B-cell lineage, multiple myeloma, renal cell cancers, nasopharyngeal cancers, thymic cancers, head and neck cancers, and gliomas. In some embodiments, the CD70+ cancer is a hematologic malignancy. In some embodiments, the CD70+ cancer is Non-Hodgkin lymphoma. In some embodiments, the CD70+ cancer is diffuse large B cell lymphoma (DLBCL). In some embodiments, the CD70+ cancer is a solid tumor. In some embodiments, the CD70+ cancer is renal cell carcinoma. In some embodiments, the CD70+ cancer is clear cell renal cell carcinoma (ccRCC). In some embodiments, the CD70+ cancer is a head and neck cancer. In some embodiments, the CD70+ cancer is squamous cell carcinoma. In some embodiments, the CD70+ cancer is head and neck squamous cell carcinoma (HNSCC).

In some embodiments, the method further comprises administering an immunotherapy to the subject. In some embodiments, the immunotherapy comprises a checkpoint inhibitor. In some embodiments, the checkpoint inhibitor is selected from an antibody that specifically binds to human PD-1, human PD-L1, or human CTLA4. In some embodiments, the checkpoint inhibitor is pembrolizumab, nivolumab, cemiplimab or ipilimumab. In some embodiments, the method further comprises administering chemotherapy to the subject.

In some embodiments, the methods of treating cancer comprise administering any of the conjugates described herein or any of the pharmaceutical compositions described herein. In some embodiments, the conjugate or pharmaceutical composition is administered intravenously. In some embodiments, the conjugate or pharmaceutical composition is administered in a dose of about 0.1 mg/kg to about 12 mg/kg.

In some embodiments, a treatment outcome of the subject is improved. In some embodiments, the improved treatment outcome is an objective response selected from stable disease, a partial response or a complete response. In some embodiments, the improved treatment outcome is reduced tumor burden. In some embodiments, the improved treatment outcome is progression-free survival or disease-free survival.

In some embodiments, provided is the use of any of the conjugates described herein or any of the pharmaceutical compositions described herein for the treatment of CD70+ cancer in a subject.

In some embodiments, provided herein is a method of treating an autoimmune disease, comprising administering to a subject in need thereof a therapeutically effective amount of any of the conjugates described herein or any of the pharmaceutical compositions described herein. In some embodiments, the autoimmune disease is rheumatoid arthritis, multiple sclerosis, or systemic lupus erythematosus. In some embodiments, the methods further comprise administering an immunosuppressive therapy to the subject. In some embodiments, method comprises administering any of the conjugates described herein or any of the pharmaceutical compositions described herein.

In some embodiments, the conjugate or pharmaceutical composition is administered intravenously. In some embodiments, the conjugate or pharmaceutical composition is administered in a dose of about 0.1 mg/kg to about 12 mg/kg. In some embodiments, a treatment outcome of the subject is improved. In some embodiments, the improved treatment outcome is a reduction in disease progression or alleviation of disease severity.

In some embodiments, provided is the use of any of the conjugates described herein or any of the pharmaceutical compositions described herein for the treatment of an autoimmune disease in a subject.

These and other aspects of the present invention may be more fully understood by reference to the following detailed description, non-limiting examples of specific embodiments and the appended drawings.

### FIGURES

FIG. 1. 2E7 and 2E7-LD038 binding assay on Caki-1.

FIG. 2. 2E7 and 2E7-LD038 binding assay on 786-O.

FIG. 3. 2E7 and 2E7-LD038 binding assay on Raji.

FIG. 4. 2E7 and 2E7-LD038 binding assay on MCF-7.

FIG. 5. In vitro blockade of CD27 binding to Caki-1 cells by 2E7 or 2E7-LD038.

FIG. 6. In vitro blockade of CD27 binding to 786-O cells by 2E7 or 2E7-LD038.

FIG. 7. In vitro blockade of CD27 binding to Raji cells by 2E7 or 2E7-LD038.

FIG. 8. 2E7 internalization in tumor cells.

FIG. 9. 2E7-LD038 internalization in tumor cells.

FIG. 10. 2E7 and 2E7-LD038 PK in rat.

FIG. 11. In vitro cell cytotoxicity of 2E7-conjugates on 786-O.

FIG. 12. In vitro cell cytotoxicity of 2E7-conjugates on Raji.

FIG. 13. In vitro cell cytotoxicity of 2E7-conjugates on Caki-1.

FIG. 14. In vitro cell cytotoxicity of 2E7-conjugates on A498.

FIG. 15. In vitro cell cytotoxicity of 2E7 and 2E7-LD038 on Caki-1.

FIG. 16. In vitro cell cytotoxicity of 2E7 and 2E7-LD038 on 786-O.

FIG. 17. In vitro cell cytotoxicity of 2E7 and 2E7-LD038 on Raji.

FIG. 18. Multiple dose study of antitumor activity of 2E7 conjugates with Caki-1.

FIG. 19. Single dose study of antitumor activity of 2E7 conjugates with Caki-1.

FIG. 20. Multiple dose study of antitumor activity of 2E7 conjugates with Raji.

FIG. 21. Single dose study of antitumor activity of 2E7 conjugates with Raji.

FIG. 22. Single dose study of antitumor activity of 2E7 conjugates with HONE-1.

FIG. 23. Single and multiple dose study of antitumor activity of 2E7-LD038 conjugates on Caki-1.

FIG. 24. Single dose study of antitumor activity of 2E7-LD038 conjugates on 786-O.

FIG. 25. Single and multiple dose study of antitumor activity of 2E7-LD038 conjugates on Raji.

FIG. 26. Single dose study of antitumor activity of 2E7-LD038 conjugates on Raji.

FIG. 27. Single dose study of antitumor activity of 2E7-LD038 conjugates in a patient derived xenograft model of DLBCL.

FIG. 28. Single dose study of antitumor activity of 2E7-LD038 conjugates in a patient derived xenograft model of DLBCL.

FIG. 29. Single dose study of antitumor activity of 2E7-LD038 conjugates in a patient derived xenograft model of DLBCL.

FIG. 30. Single dose study of antitumor activity of 2E7-LD038 conjugates in a patient derived xenograft model of DLBCL.

FIG. 31. Single dose study of antitumor activity of 2E7-LD038 conjugates in a patient derived xenograft model of DLBCL.

FIG. 32. Single dose study of antitumor activity of 2E7-LD038 conjugates in a patient derived xenograft model of ccRCC.

FIG. 33. Single dose study of antitumor activity of 2E7-LD038 conjugates in a patient derived xenograft model of ccRCC.

FIG. 34. Single dose study of antitumor activity of 2E7-LD038 conjugates in a patient derived xenograft model of Head and Neck cancer.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

CD70 is a cell surface antigen on activated, but not on resting, T and B lymphocytes. It is also referred to as CD27L, Tumor Necrosis Factor (Ligand) Superfamily, Member 7, TNFSF7, Surface Antigen CD70, and Ki-24 Antigen. It is reported to be overexpressed on certain cancers, as further described herein. Human CD70 polypeptides include, but are not limited to, those having the amino acid sequences set forth in UniProt identifiers P32970-1 and P32970-2 and RefSeq NP_001243.1 and NP_001317261.1; these sequences are incorporated by reference herein.

As used herein, an “epitope” refers to the amino acids conventionally bound by an immunoglobulin VH/VL pair, such as the antibodies or antigen binding portions thereof. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody or antigen binding portions thereof, and thus represents the target of specificity of an antibody or antigen binding portion thereof. In the case of a single domain antibody, an epitope represents the unit of structure bound by a variable domain in isolation.

As used herein, “specifically binds” refers to the ability of a Binding unit (e.g., an antibody or antigen binding portion thereof) described herein to bind to a target, such as human CD70, with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the Binding unit and the concentration of target polypeptide. The person of ordinary skill in the art can determine appropriate conditions under which the Binding unit described herein selectively bind to CD70 using any suitable methods, such as titration of an antibody or antigen binding portion thereof in a suitable cell binding assay. A Binding unit specifically bound to CD70 is not displaced by a non-similar competitor. In certain embodiments, a Binding unit is said to specifically bind to CD70 when it preferentially recognizes its target antigen, CD70, in a complex mixture of proteins and/or macromolecules.

In some embodiments, the Binding unit as described herein specifically binds to a CD70 polypeptide with a dissociation constant (KD or K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, the Binding unit as described herein specifically binds to a CD70 polypeptide with a dissociation constant (KD) of from about 10⁻⁵ M to 10⁻⁶ M. In some embodiments, the Binding unit as described herein specifically binds to a CD70 polypeptide with a dissociation constant (KD) of from about 10⁻⁶ M to 10⁻⁷ M. In some embodiments, the Binding unit as described herein specifically binds to a CD70 polypeptide with a dissociation constant (KD) of from about 10⁻⁷ M to 10⁻⁸ M. In some embodiments, the Binding unit as described herein specifically binds to a CD70 polypeptide with a dissociation constant (KD) of from about 10⁻⁸ M to 10⁻⁹ M. In some embodiments, the Binding unit as described herein specifically binds to a CD70 polypeptide with a dissociation constant (KD) of from about 10⁻⁹ M to 10⁻¹⁰ M. In some embodiments, the Binding unit as described herein specifically binds to a CD70 polypeptide with a dissociation constant (KD) of from about 10⁻¹⁰ M to 10⁻¹¹ M. In some embodiments, the Binding unit as described herein specifically binds to a CD70 polypeptide with a dissociation constant (KD) of from about 10⁻¹¹ M to 10⁻¹² M. In some embodiments, the Binding unit as described herein specifically binds to a CD70 polypeptide with a dissociation constant (KD) of less than 10⁻¹² M.

Unless otherwise indicated, the term “alkyl” by itself or as part of another term refers to a substituted or unsubstituted straight chain or branched, saturated hydrocarbon having the indicated number of carbon atoms (e.g., “—C₁-C₅ alkyl”, “—C₁-C₅ alkyl” or “—C₁-C₁₀” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C₂-C₈ substituted or unsubstituted straight chain or branched, hydrocarbon with at least one site of unsaturation (i.e., a carbon-carbon, sp² double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C₂-C₈, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃, —. Up to two heteroatoms may be consecutive, such as, for example, —CH₂NHOCH₃ and CH₂OSi(CH₃)₃. In some embodiments, a C₁ to C₄ heteroalkyl has 1 to 4 carbon atoms and 1 or 2 heteroatoms and a C₁ to C₃ heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH₂CH₂SCH₂CH₂— and —CH₂SCH₂CH₂NHCH₂—. In some embodiments, a C₁ to C₄ heteroalkylene has 1 to 4 carbon atoms and 1 or 2 heteroatoms and a C₁ to C₃ heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C₂ to C₄ heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C₃-C₈ carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo”, by itself or as part of another term, refers to a substituted or unsubstituted C₃-C₈ carbocycle group defined above wherein another of the carbocycle groups' hydrogen atoms is replaced with a bond (i.e., it is divalent).

Unless otherwise indicated, a “C₃-C₁₀ carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C₃-C₁₀ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C₃-C₁₀ carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C₃-C₈ heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C₃-C₈ heterocyclo”, by itself or as part of another term, refers to a substituted or unsubstituted C₃-C₈ heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO⁻M⁺, where M⁺ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R¹⁰, —O⁻, —OR¹⁰, —SR¹⁰, —S⁻, —NR¹⁰₂, —NR¹⁰₃, ═NR¹⁰, —CX₃, —CN, —OCN, —SCN, —N═C═O, —NCS, —NO, —NO₂, ═N₂, —N₃, —NR¹⁰C(═O)R¹⁰, —C(═O)R¹⁰, —C(═O)NR¹⁰₂, —SO₃⁻, —SO₃H, —S(═O)₂R¹⁰, —OS(═O)₂OR¹⁰, —S(═O)₂NR¹⁰, —S(═O)R¹⁰, —OP(═O)(OR¹⁰)₂, —P(═O)(OR¹⁰)₂, —PO⁻₃, —PO₃H₂, —AsO₂H₂, —C(═O)R¹⁰, —C(═O)X, —C(═S)R¹⁰, —CO₂R¹⁰, —CO₂⁻, —C(═S)OR¹⁰, C(═O)SR¹⁰, C(═S)SR¹⁰, C(═O)NR¹⁰₂, C(═S)NR¹⁰₂, or C(═NR¹⁰)NR¹⁰₂, where each X is independently a halogen: —F, —Cl, —Br, or —I; and each R¹⁰ is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkyl heteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any one of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH₂, —N(R′)(R″), —N(R″)₃, ═NR, —CX₃, —CN, —NO₂, —NR′C(═O)H, —NR′C(═O)R, —NR′C(═O)R″, —C(═O)R′, —C(═O)NH₂, —C(═O)N(R′)R″, —S(═O)₂R″, —S(═O)₂NH₂, —S(═O)₂N(R′)R″, —S(═O)₂NH₂, —S(═O)₂N(R′)R″, —S(═O)₂OR′, —S(═O)R″, —OP(═O)(OR′)(OR″), —OP(OH)₃, —P(═O)(OR′)(OR″), —PO₃H₂, —C(═O)R′, —C(═S)R″, —CO₂R′, —C(═S)OR″, —C(═O)SR′, —C(═S)SR′, —C(═S)NH₂, —C(═S)N(R′)(R″)₂, —C(═NR′)NH₂, —C(═NR′)N(R′)R″, and salts thereof, wherein each X is independently selected from the group consisting of a halogen: —F, —Cl, —Br, and —I; and wherein each R″ is independently selected from the group consisting of C₁-C₂₀ alkyl, C₂-C₂₀ alkenyl, C₂-C₂₀ alkynyl, C₆-C₂₄ aryl, C₃-C₂₄ heterocyclyl (including C₅-C₂₄ heteroaryl), a protecting group, and a prodrug moiety or two of R″ together with the heteroatom to which they are attached defines a heterocyclyl; and R′ is hydrogen or R″, wherein R″ is selected from the group consisting of C₁-C₂₀ alkyl, C₆-C₂₄ aryl, C₃-C₂₄ heterocyclyl (including C₅-C₂₄ heteroaryl), and a protecting group.

Typically, optional substituents are selected from the group consisting of —X, —OH, —OR″, —SH, —SR″, —NH₂, —NH(R″), —NR′(R″)₂, —N(R″)₃, ═NH, ═NR″, —CX₃, —CN, —NO₂, —NR′C(═O)H, NR′C(═O)R″, —CO₂H, —C(═O)H, —C(═O)R″, —C(═O)NH₂, —C(═O)NR′R″—, —S(═O)₂R″, —S(═O)₂NH₂, —S(═O)₂N(R′)R″, —S(═O)₂NH₂, —S(═O)₂N(R′)(R″), —S(═O)₂OR′, —S(═O)R″, —C(═S)R″, —C(═S)NH₂, —C(═S)N(R′)R″, —C(═NR′)N(R″)₂, and salts thereof, wherein each X is independently selected from the group consisting of —F and —Cl, R″ is typically selected from the group consisting of C₁-C₆ alkyl, C₆-C₁₀ aryl, C₃-C₁₀ heterocyclyl (including C₅-C₁₀ heteroaryl), and a protecting group; and R′ independently is hydrogen, C₁-C₆ alkyl, C₆-C₁₀ aryl, C₃-C₁₀ heterocyclyl (including C₅-C₁₀ heteroaryl), and a protecting group, independently selected from R″. More typically, substituents are selected from the group consisting of —X, —R″, —OH, —OR″, —NH₂, —NH(R″), —N(R″)₂, —N(R″)₃, —CX₃, —NO₂, —NHC(═O)H, —NHC(═O)R″, —C(═O)NH₂, —C(═O)NHR″, —C(═O)N(R″)₂, —CO₂H, —CO₂R″, —C(═O)H, —C(═O)R″, —C(═O)NH₂, —C(═O)NH(R″), —C(═O)N(R″)₂, —C(═NR′)NH₂, —C(═NR′)NH(R″), —C(═NR′)N(R″)₂, a protecting group and salts thereof, wherein each X is —F, R″ is independently selected from the group consisting of C₁-C₆ alkyl, C₆-C₁₀ aryl, C₅-C₁₀ heteroaryl and a protecting group; and R′ is selected from the group consisting of hydrogen, C₁-C₆ alkyl and a protecting group, independently selected from R″.

The phrase “pharmaceutically acceptable salt,” as used herein, refers to pharmaceutically acceptable organic or inorganic salts of a compound (e.g., a Linker, Drug Linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Although structures shown throughout the specification are depicted with specific stereocenters, the specification should be read to include variations in those stereocenters. For example, the structure of exatecan may be shown in the (S,S) configuration, but the (R,S) diastereomer of exatecan is also envisioned as being found in a separate embodiment of a conjugate as described herein.

Other terms are defined herein within the description of the various aspects of the invention.

### DETAILED DESCRIPTION

Provided herein are CD70 antibody drug conjugates (ADCs) that specifically bind to human CD70. The CD70 antibody drug conjugates comprise a Binding unit comprising one or more CD70 antibodie(s) or antigen binding portions thereof, a Linker, and one or more Drug unit(s) such as cytotoxic agents or immune modulatory agents. In some embodiments, the CD70 ADCs specifically bind to and reduce the number of CD70+ cells in a subject. In some embodiments, the CD70 ADCs specifically bind to and reduce the number of CD70+ cancer cells in a subject. In some embodiments, the CD70 ADCs specifically bind to and reduce the number of CD70+ cells associated with a disease or condition in a subject, such as an autoimmune disease.

In some embodiments, the Binding unit of the CD70 ADCs comprises a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, the Binding unit of the CD70 ADCs comprises a heavy chain variable region (VH) and a light chain variable region (VL), the VH and VL regions having the amino acid sequences set forth in SEQ ID NO:3 and SEQ ID NO:4, respectively. In some embodiments, the Binding unit of the CD70 ADCs comprises a heavy chain variable region (VH) and a light chain variable region (VL), the VH and VL regions having the amino acid sequences set forth in SEQ ID NO:5 and SEQ ID NO:6, respectively. In some embodiments, the Binding unit of the CD70 ADCs comprises a heavy chain variable region (VH) and a light chain variable region (VL), the VH and VL regions having the amino acid sequences set forth in SEQ ID NO:7 and SEQ ID NO:8, respectively. In some embodiments, the Binding unit of the CD70 ADCs comprises a heavy chain variable region (VH) and a light chain variable region (VL), the VH and VL regions having the amino acid sequences set forth in SEQ ID NO:9 and SEQ ID NO:10, respectively. In some embodiments, the Binding unit of the CD70 ADCs comprises a heavy chain variable region (VH) and a light chain variable region (VL), the VH and VL regions having the amino acid sequences set forth in and SEQ ID NO:11 and SEQ ID NO:12; respectively.

In some embodiments, the Binding unit of the CD70 ADCs comprises a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. The phrase “wherein the CDRs of the heavy or light chain variable regions are not modified” refers to the VH and VL CDRs that do not have amino acid substitutions, deletions or insertions, as compared to the recited amino acid sequences.

In some embodiments, the Binding unit of the CD70 ADCs comprises a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit of the CD70 ADCs comprises a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit of the CD70 ADCs comprises a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit of the CD70 ADCs comprises a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit of the CD70 ADCs comprises a heavy chain variable region (VH) and a light chain variable region (VL), the VH and VL regions having the amino acid sequences set forth in and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding unit of the CD70 ADCs comprises a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit comprises a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the Binding unit specifically binds to CD70. In some embodiments, the Binding unit comprises a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the Binding unit specifically binds to CD70 with a higher binding affinity (lower Kd) than that of antibody 69A7. In some embodiments, the Binding unit comprises a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, the Binding unit includes a CD70 antibody or antigen binding portion(s) thereof and can optionally include other peptides or polypeptides covalently attached to the CD70 antibody or antigen binding portion thereof. In any of these embodiments, the Binding unit specifically binds to CD70.

In some embodiments, the Binding unit comprises a heavy chain variable region (VH) and a light chain variable region (VL), the VH and VL regions having the amino acid sequences set forth in SEQ ID NO:3 and SEQ ID NO:4, respectively; wherein the Binding unit specifically binds to CD70. In some embodiments, the Binding unit comprises a heavy chain variable region (VH) and a light chain variable region (VL), the VH and VL regions having amino acid sequences set forth SEQ ID NO:3 and SEQ ID NO:4, respectively; wherein the Binding unit specifically binds to CD70 with a higher binding affinity (lower Kd) than that of antibody 69A7. In some embodiments, the Binding unit comprises a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, the Binding unit comprises a heavy chain variable region (VH) and a light chain variable region (VL), the VH and VL regions having the amino acid sequences set forth in SEQ ID NO:5 and SEQ ID NO:6, respectively; wherein the Binding unit specifically binds to CD70. In some embodiments, the Binding unit comprises a heavy chain variable region (VH) and a light chain variable region (VL), the VH and VL regions having the amino acid sequences set forth in SEQ ID NO:5 and SEQ ID NO:6, respectively; wherein the Binding unit specifically binds to CD70 with a higher binding affinity (lower Kd) than that of antibody 69A7. In some embodiments, the Binding unit comprises a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, the Binding unit comprises a heavy chain variable region (VH) and a light chain variable region (VL), the VH and VL regions having the amino acid sequences set forth in SEQ ID NO:7 and SEQ ID NO:8, respectively; wherein the Binding unit specifically binds to CD70. In some embodiments, the Binding unit comprises a heavy chain variable region (VH) and a light chain variable region (VL), the VH and VL regions having the amino acid sequences set forth in SEQ ID NO:7 and SEQ ID NO:8, respectively; wherein the Binding unit specifically binds to CD70 with a higher binding affinity (lower Kd) than that of antibody 69A7. In some embodiments, the Binding unit comprises a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, the Binding unit comprises a heavy chain variable region (VH) and a light chain variable region (VL), the VH and VL regions having the amino acid sequences set forth in SEQ ID NO:9 and SEQ ID NO:10, respectively; wherein the Binding unit specifically binds to CD70. In some embodiments, the Binding unit comprises a heavy chain variable region (VH) and a light chain variable region (VL), the VH and VL regions having the amino acid sequences set forth in SEQ ID NO:9 and SEQ ID NO:10, respectively; wherein the Binding unit specifically binds to CD70 with a higher binding affinity (lower Kd) than antibody 69A7. In some embodiments, the Binding unit comprises a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, the Binding unit comprises a heavy chain variable region (VH) and a light chain variable region (VL), the VH and VL regions having the amino acid sequences set forth in SEQ ID NO:11 and SEQ ID NO:12; respectively; wherein the Binding unit specifically binds to CD70. In some embodiments, the Binding unit comprises a heavy chain variable region (VH) and a light chain variable region (VL), the VH and VL regions having the amino acid sequences set forth in SEQ ID NO:11 and SEQ ID NO:12; respectively; wherein the Binding unit specifically binds to CD70 with a higher binding affinity (lower Kd) than that of antibody 69A7. In some embodiments, the Binding unit comprises a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, a Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the compositions and methods described herein relate to reduction of CD70+ cells in a subject (e.g., reducing the number of CD70+ cells in a cancer or tumor, or CD70+ cells associated with an autoimmune disease or disorder) by a CD70 ADC in vivo. In some embodiments, the compositions and methods described herein relate to the treatment of CD70+ cancer in a subject by administering a CD70 ADC. In some embodiments, the compositions and methods described herein relate to the treatment of an autoimmune disorder in a subject by administering a CD70 ADC. In some embodiments, the compositions and methods described herein relate to the treatment of disease or disorder associated with CD70+ cells in a subject by administering a CD70 ADC. In any of these embodiments, the methods further include a reduction in the number of CD70+ cells in the subject that are associated with the disease, condition or cancer.

As used herein, the term “antibody” refers to immunoglobulin molecules and immunologically active portions of immunoglobulin molecules, i.e., molecules that contain an antigen binding site(s) that specifically binds to an antigen, e.g., human CD70. The term generally refers to antibodies comprised of two immunoglobulin heavy chain variable regions and two immunoglobulin light chain variable regions including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH₄. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of a CD70 antibody refers to the portions of a CD70 antibody as described herein having the VH and VL sequences of the CD70 antibody or the CDRs of a CD70 antibody and that specifically binds to CD70. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab fragment, i.e. a monovalent fragment composed of the VL, VH, CL and CH1 domains; (ii) an F(ab′)₂ fragment, i.e. a bivalent fragment comprising two Fab fragments linked to one another in the hinge region via a disulfide bridge; and (iii) an Fv fragment composed of the VL and VH domains, in each case of a CD70 antibody. Although the two domains of the Fv fragment, namely VL and VH, are encoded by separate coding regions, they may further be linked to one another using a synthetic linker, e.g., a poly-G4S amino acid sequence (‘(G₄S)ₙ’ disclosed as SEQ ID NO: 27, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the CD70 antibodies or antigen binding portions thereof are part of a bispecific or multispecific Binding unit. Bispecific and multi-specific antibodies include the following: an scFv1-ScFv2, an ScFv1₂-Fc-scFv2₂, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in a polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to CD70.

In some embodiments, a conservatively modified variant of the CD70 antibody or antigen binding portion thereof (i.e., the Binding unit) can have an alteration(s) in the framework regions (i.e., other than in the CDRs), e.g. a conservatively modified variant of a CD70 antibody has the amino acid sequences of the VH and VL CDRs (set forth in sets of amino acid sequences SEQ ID NO:21, SEQ ID NO:22, SEQ ID NO:13, SEQ ID NO:24, SEQ ID NO:25 and SEQ ID NO:26; SEQ ID NO:21, SEQ ID NO:22, SEQ ID NO:14, SEQ ID NO:24, SEQ ID NO:25 and SEQ ID NO:26; SEQ ID NO:21, SEQ ID NO:22, SEQ ID NO:15, SEQ ID NO:24, SEQ ID NO:25 and SEQ ID NO:26; SEQ ID NO:21, SEQ ID NO:22, SEQ ID NO:23, SEQ ID NO:24, SEQ ID NO:25 and SEQ ID NO:18; and SEQ ID NO:16, SEQ ID NO:17, SEQ ID NO:23, SEQ ID NO:24, SEQ ID NO:25 and SEQ ID NO:26) and has at least one conservative amino acid substitution in a framework region (FR). In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the Binding unit (i.e., the Binding unit) exhibits specific binding to CD70.

For conservative amino acid substitutions, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to CD70.

In some embodiments, the Binding unit can be further optimized to, for example, decrease potential immunogenicity or optimize other functional property, while maintaining functional activity, for therapy in humans. In some embodiments, the Binding units comprise a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the Binding units comprise a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the Binding unit comprises a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. In some embodiments, the Binding unit may comprise a VH region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 3. In some embodiments, the Binding unit may comprise a VL region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 4.

In some embodiments, the Binding unit comprises a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. In some embodiments, the Binding unit may comprise a VH region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 5. In some embodiments, the Binding unit may comprise a VL region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 6.

In some embodiments, the Binding unit comprises a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. In some embodiments, the Binding unit may comprise a VH region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 7. In some embodiments, the Binding unit may comprise a VL region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 8.

In some embodiments, the Binding unit comprises a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. In some embodiments, the Binding unit may comprise a VH region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 9. In some embodiments, the Binding unit may comprise a VL region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 10.

In some embodiments, the Binding unit comprises a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, the Binding unit comprises a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. In some embodiments, the Binding unit may comprise a VH region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 11. In some embodiments, the Binding unit may comprise a VL region having an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 12.

In any of these embodiments, the functional activity of the Binding unit includes specifically binding to CD70. Additional functional activities include depletion of CD70+ cells (e.g., cancer cells or autoimmune cells). Additionally, a Binding unit having functional activity means the polypeptide exhibits activity similar to, or better than, the activity of a reference antibody or antigen-binding portion thereof as described herein (e.g., a reference CD70 binding antibody or antigen binding portion thereof comprising (i) a heavy chain variable region having the amino acid sequence set forth in SEQ ID NO:1 and (ii) a light chain variable region having the amino acid sequence set forth in SEQ ID NO:2 or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of the Binding unit preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g. BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, the Binding unit has fully human constant regions. In some embodiments, the Binding unit has humanized constant regions. In some embodiments, the Binding unit has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAQ1, and IgA2. The heavy-chain constant regions (Fc) that correspond to the different classes of immunoglobulins can be α, δ, ε, γ, and μ, respectively. The light chains can be one of either kappa (or κ) and lambda (or λ).

A constant region can have an IgG1 isotype. A constant region can have an IgG2 isotype. A constant region can have an IgG3 isotype. A constant region can have an IgG4 isotype. An Fc domain can have a hybrid isotype comprising constant regions from two or more isotypes. An immunoglobulin constant region can be an IgG1 or IgG4 constant region. In some embodiments, the CD70 antibody heavy chain of the Binding unit is of the IgG1 isotype and has the amino acid sequence set forth in SEQ ID NO:28. In some embodiments, the CD70 antibody light chain of the Binding unit is of the kappa isotype and has the amino acid sequence set forth in SEQ ID NO:29.

### Fc Domain Modifications to Alter Effector Function

In some embodiments, an Fc region or Fc domain of the Binding unit has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A Binding unit with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A Binding unit with diminished binding to FcRs can be prepared containing such amino acid modifications.

In some embodiments, the Binding unit comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Binding Units

In various embodiments, the Binding units can be produced in human, murine or other animal-derived cells lines. Recombinant DNA expression can be used to produce Binding units. This allows the production of CD70 antibodies as well as a spectrum of CD70 antigen binding portions in a host species of choice. The production of Binding units in bacteria, yeast, transgenic animals and chicken eggs are also alternatives for cell-based production systems. The main advantages of transgenic animals are potential high yields from renewable sources.

In some embodiments, a VH polypeptide having the amino acid sequence set forth in SEQ ID NOs:3, 5, 7, 9 or 11 is encoded by a nucleic acid. In some embodiments, a VH polypeptide having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 3, 5, 7, 9 or 11 is encoded by a nucleic acid. In some embodiments, a VL polypeptide having the amino acid sequence set forth in SEQ ID NOs: 4, 6, 8, 10, or 12 is encoded by a nucleic acid. In some embodiments, a VL polypeptide having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 4, 6, 8, 10, or 12 is encoded by a nucleic acid. In some embodiments, a nucleic acid encodes a VH polypeptide having the amino acid sequence set forth in SEQ ID NOs:3, 5, 7, 9 or 11. In some embodiments, a nucleic acid encodes a VH polypeptide having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs:3, 5, 7, 9 or 11. In some embodiments, a nucleic acid encodes a VL polypeptide having the amino acid sequence set forth in SEQ ID NOs: 4, 6, 8, 10, or 12. In some embodiments, a nucleic acid encodes a VL polypeptide having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 4, 6, 8, 10, or 12. In some embodiments, the nucleic acid encodes a VH polypeptide having the amino acid sequence set forth in SEQ ID NO:3. In some embodiments, the nucleic acid encodes a VH polypeptide having the amino acid sequence set forth in SEQ ID NO:5. In some embodiments, the nucleic acid encodes a VH polypeptide having the amino acid sequence set forth in SEQ ID NO:7. In some embodiments, the nucleic acid encodes a VH polypeptide having the amino acid sequence set forth in SEQ ID NO:9. In some embodiments, the nucleic acid encodes a VH polypeptide having the amino acid sequence set forth in SEQ ID NO:11. In some embodiments, the nucleic acid encodes a VH polypeptide having the amino acid sequence set forth in SEQ ID NO:4. In some embodiments, the nucleic acid encodes a VH polypeptide having the amino acid sequence set forth in SEQ ID NO:6. In some embodiments, the nucleic acid encodes a VH polypeptide having the amino acid sequence set forth in SEQ ID NO:8. In some embodiments, the nucleic acid encodes a VH polypeptide having the amino acid sequence set forth in SEQ ID NO:10. In some embodiments, the nucleic acid encodes a VH polypeptide having the amino acid sequence set forth in SEQ ID NO:12.

In some embodiments, the nucleic acid encodes VH and VL polypeptides having the amino acid sequences set forth in SEQ ID NOs:3 and 4. In some embodiments, the nucleic acid encodes VH and VL polypeptides having the amino acid sequences set forth in SEQ ID NOs:5 and 6. In some embodiments, the nucleic acid encodes VH and VL polypeptides having the amino acid sequences set forth in SEQ ID NOs:7 and 8. In some embodiments, the nucleic acid encodes VH and VL polypeptides having the amino acid sequences set forth in SEQ ID NOs:9 and 10. In some embodiments, the nucleic acid encodes VH and VL polypeptides having the amino acid sequences set forth in SEQ ID NOs:11 and 12.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of a Binding units can be prepared by a variety of methods known in the art. These methods include, but are not limited to, preparation of synthetic nucleotide sequences encoding of a CD70 antibody antigen binding portion thereof. In addition, oligonucleotide-mediated (or site-directed) mutagenesis, PCR-mediated mutagenesis, and cassette mutagenesis can be used to prepare nucleotide sequences encoding a CD70 antibody or antigen binding portion thereof. A nucleic acid sequence encoding at least a CD70 antibody or antigen binding portion thereof, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode a CD70 antibody or antigen binding portion thereof or a VH or VL polypeptide thereof (i.e., Binding unit).

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., a CD70 antibody or antigen binding portion thereof (i.e., a Binding unit)) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a CD70 antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a CD70 antibody or antigen binding portion thereof as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant CD70 antibodies or antigen-binding portions thereof. Known glycolytic genes can also provide very efficient transcriptional control signals. For example, the promoter and terminator signals of the phosphoglycerate kinase gene can be utilized.

Production of Binding units in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding a CD70 antibody or antigen binding portion thereof or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding Binding units include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

For expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the CD70 variable region(s) or antigen binding portions thereof are then transfected singly with nucleotides encoding a CD70 antibody or an antibody polypeptide or antigen-binding portion thereof, or are co-transfected with a polynucleotide(s) encoding VH and VL chain coding regions. The transfected recipient cells are cultured under conditions that permit expression of the incorporated coding regions and the expressed antibody chains or intact antibodies or antigen binding portions are recovered from the culture.

The nucleic acids containing the coding regions encoding a Binding unit can be assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing CD70 antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the Binding units, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding a Binding unit can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled Binding units. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of Binding units in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

One or more Binding units can be produced in vivo in an animal that has been engineered or transfected with one or more nucleic acid molecules encoding the polypeptides, according to any suitable method.

An antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled Binding units or enhanced stability of the transfected cell lines.

Additionally, plants have emerged as a convenient, safe and economical alternative expression system for recombinant antibody production, which are based on large scale culture of microbes or animal cells. Binding units can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the CD70 antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). A CD70 binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to CD70. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, the Binding unit comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, the Binding unit is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be Binding units (see, e.g. US 2006/0025576A1).

In some embodiments, the Binding units (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the Binding units comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the Binding unit a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, the Binding unit is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, the Binding unit is a bispecific antibody is composed of a single polypeptide chain comprising two single chain FV portions (scFV) fused to each other by a peptide linker.

In some embodiments, the Binding unit is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

Igg-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof (i.e., Binding units) can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure Binding units of at least about 90% to 95% homogeneity are advantageous, as are those with 98% to 99% or more homogeneity, particularly for pharmaceutical uses. Once purified, partially or to homogeneity as desired, an intact Binding unit can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Antibody Drug Conjugates

In some embodiments, a CD70 antibody drug conjugate (also referred to as a CD70 conjugate or CD70 ADC) comprises a Binding unit comprising a CD70 antibody or antigen binding portion attached to at least one Linker and at least one Drug unit is attached to each linker. As used herein the term “Drug unit” refers to a cytotoxic agent (such as a chemotherapeutic agent or drug), immunomodulatory agent, nucleic acid (including siRNAs), growth inhibitory agent, toxin (e.g., protein toxins, enzymatically active toxins of bacterial, fungal, plant, or animal origin, or fragments thereof), radioactive isotope, PROTAC and other compound that is active against target cells when delivered to those cells.

### Cytotoxic Agents

In some embodiments, a CD70 ADC includes at least one Drug unit that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HT1-286.

Other tubulin disrupting agents include taccalonolide A, taccalonolide B, taccalonolide AF, taccalonolide AJ, taccalonolide Al-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan (SS form), a diastereoisomer of exatecan, the RS form, and the exatecan analog DXd (see US20150297748) containing exatecan) and an analog of DXd containing the RS diastereoisomer of exatecan. Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, the Drug unit is an immune modulatory agent. An immune modulatory agent can be, for example, a TLR7 and/or TLR8 agonist, a STING agonist, a RIG-I agonist or other immune modulatory agent.

In some embodiments, the Drug unit is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164, US 20150299194, US20110098248, US20100143301, and US20090047249.

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755, WO2017216054, WO2017190669, WO2017202704, WO2017202703, WO20170071944, US20140045849, US20140073642, WO2014056953, WO2014076221, WO2014128189, US20140350031, WO2014023813, US20080234251, US20080306050, US20100029585, US20110092485, US20110118235, US20120082658, US20120219615, US20140066432, US20140088085, US20140275167, and US20130251673, WO2018198091, and US20170131421.

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-I agonist. Examples of RIG-I agonists include KIN1148, SB-9200, KIN700, KIN600, KIN500, KIN100, KIN101, KIN400 and KIN2000.

### Toxins

In some embodiments, the Drug unit is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, the Drug unit is a radioactive atom. A variety of radioactive isotopes are available for the production of radioconjugates. Examples include I131, I125, Y90, Re186, Re188, Sm153, Bi213, P32, Pb212 and radioactive isotopes of Lutetium (e.g., Lu177).

### PROTACs

In some embodiments, the Drug unit is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The CD70 conjugates typically comprise at least one Linker, each Linker having at least one Drug unit attached to it. Typically, a conjugate includes a Linker between a CD70 antibody (or antigen binding portion thereof (i.e., a Binding unit) and the Drug unit. In various embodiments, the Linker may comprise a protease cleavable linker, an acid-cleavable linker, a disulfide linker, a disulfide-containing linker, or a disulfide-containing linker having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690 and WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, the Linker is a cleavable linker that is cleavable under intracellular conditions, such that cleavage of the linker releases the Drug unit from the Binding unit and/or Linker in the intracellular environment. For example, in some embodiments, the Linker is cleavable by a cleaving agent that is present in the intracellular environment (e.g., within a lysosome or endosome or caveolae). A Linker can comprise, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, for example, in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly (SEQ ID NO: 35) linker (see, e.g., US2015/0297748). One advantage of using intracellular proteolytic release of the Drug unit is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable Linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether Linker (such as, for example, a thioether attached to the drug via an acyl hydrazone bond (see, e.g., U.S. Pat. No. 5,622,929)).

In some embodiments, the Linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the Linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the Linker unit is not cleavable, such as a maleimidocaproyl linker, and the drug is released by antibody degradation. (See U.S. Publication No. 2005/0238649).

In some embodiments, the Linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC), are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, the Linker promotes cellular internalization. In some embodiments, the Linker promotes cellular internalization when conjugated to the drug such as a cytotoxic agent (i.e., in the milieu of the linker-drug moiety of the ADC as described herein). In yet other embodiments, a Linker promotes cellular internalization when conjugated to both the drug and the CD70 antibody (i.e., in the milieu of the ADC as described herein).

In other embodiments, CD70 ADCs may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to a Binding unit have been described in, for example WO94/11026.

The conjugates of a CD70 ADCs include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In some embodiments, the Linker is attached to a terminus of an amino acid sequence of an antibody or antigen binding portion thereof (i.e., a Binding unit) or can be attached to a side chain modification of an antibody or antigen binding portion thereof, such as the side chain of a lysine, serine, threonine, cysteine, tyrosine, aspartic acid, a non-natural amino acid residue, glutamine, or glutamic acid residue. An attachment between an antibody or antigen binding portion thereof and a Linker or Drug unit can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, the Linker is attached to the Binding unit at an interchain disulfide. In some embodiments, the Linker is connected to the Binding unit at a hinge cysteine residue. In some embodiments, the Linker is attached to the Binding unit at an engineered cysteine residue. In some embodiments, the Linker is connected to the Binding unit at a lysine residue. In some embodiments, the Linker is connected to the Binding unit at an engineered glutamine residue. In some embodiments, the Linker is connected to the Binding unit at an unnatural amino acid engineered into the heavy chain.

In some embodiments, the Linker is attached to the Binding unit via a sulfhydryl group. In some embodiments, the Linker is attached to the Binding unit via a primary amine. In some embodiments, the Linker is attached via a link created between an unnatural amino acid on the Binding unit by reacting with oxime bond that was formed by modifying a ketone group with an alkoxyamine on a drug.

In some embodiments, the Linker is attached to the Binding unit via Sortase A linker. A Sortase A linker can be created by a Sortase A enzyme fusing an LPXTG recognition motif (SEQ ID NO: 33) to an N-terminal GGG motif to regenerate a native amide bond.

In some embodiments, the Linker has the following formula (I):

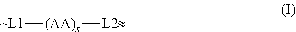

or a salt thereof, wherein:
L1 is a Stretcher unit having an attachment site for a Binding unit;
AA is an Amino Acid unit having from 1 to 12 subunits;
s is 0 or 1;
L2 is a Linker Subunit having from 1 to 4 attachment sites for a Drug unit;
the wavy (˜) line indicates an attachment site for the Binding unit, and the double wavy (≈) line indicates an attachment site for a Drug unit;
wherein at least one Polar unit is present within the Amino Acid unit, the Stretcher unit, the Linker Subunit, or combinations thereof, and wherein the Polar unit(s) is selected from Sugar units, PEG units, Carboxyl units, and combinations thereof.

In some embodiments, the Linker has the following formula (I):

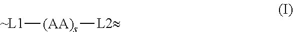

or a salt thereof, wherein:
L1 is a Stretcher unit having an attachment site for a Binding unit;
AA is an Amino Acid unit having from 1 to 12 subunits;
s is 0 or 1;
L2 is a Linker Subunit having from 1 to 4 attachment sites for a Drug unit;
the wavy (˜) line indicates an attachment site for the Binding unit, and the double wavy (≈) line indicates an attachment site for a Drug unit;
wherein at least one Polar unit is present within the Amino Acid unit, the Linker Subunit, the Stretcher unit, or combinations thereof, and wherein the Polar unit(s) is selected from Sugar units, PEG units, Carboxyl units, and combinations thereof.

### Sugar Units

In some embodiments, the Linker comprises a Sugar unit that has the following formula:

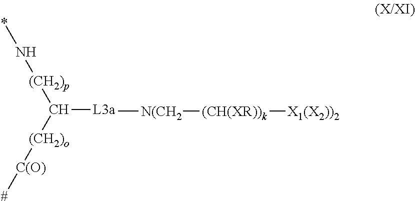

or a salt thereof, wherein:
   each X is independently selected from NH or 0;
   each R is independently selected from hydrogen, acetyl, a monosaccharide, a
   disaccharide, and a polysaccharide;
   each X₁ is independently selected from CH₂ and C(O);
   each X₂ is independently selected from H, OH and OR;
   k is 1 to 10; and
   L3a is selected from C₁-C₁₀ alkylene and polyethylene glycol having from 1 to 24 ethylene glycol subunits;
   p and o are independently 0 to 2; and
   each * and each # indicate an attachment site for another subunit of an Amino Acid unit (AA), a Linker subunit L2, or a Stretcher unit (L1).

In some embodiments, the Linker comprises a Sugar unit having a formula selected from:

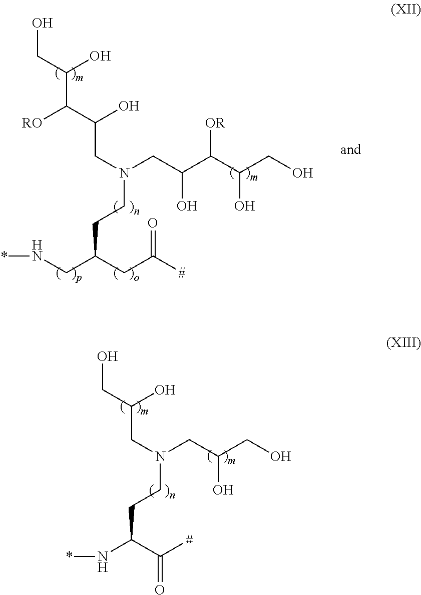

or a stereoisomer or salt thereof, wherein:
   each R is independently selected from hydrogen, a monosaccharide, a disaccharide and a polysaccharide;
   p and o are independently 0 to 2;
   m is 1-8;
   n is 0 to 4; and
   each * and each # indicate an attachment site for another subunit of the Amino Acid unit (AA), the Linker subunit L2, or the Stretcher unit (L1).

### PEG Units

In some embodiments, the Linker comprises a PEG unit having a formula selected from:
(a)

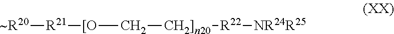

or a salt thereof, wherein:
   R²⁰ is a functional group for attachment to a subunit of the Amino Acid unit, a Stretcher unit, and/or a portion of the Linker Subunit L2;
   R²¹ and R²² are each, independently, optional C₁-C₃ alkylene;
   R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle; provided that both R²⁴ and R²⁵ are not H;
   the wavy line (˜) indicates the attachment site to R²⁰; and
   n20 is 1 to 26;
   or
(b)

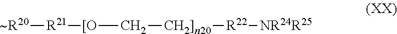

or a salt thereof, wherein:
R²⁰ is a functional group for attachment to a subunit of the Amino Acid unit, a Stretcher unit and/or a portion of the Linker Subunit L2;
R²¹ and R²² are each, independently, optional C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is a polyethylene glycol, optionally having 1 to 24 ethylene glycol subunits;
the wavy line (˜) indicates the attachment site to R²⁰; and
n20 is 1 to 26;
or
(c)

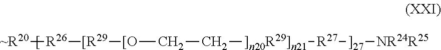

or a salt thereof, wherein:
R²⁰ is a functional group for attachment to a subunit of an Amino Acid unit, a Stretcher unit and/or a portion of a Linker Subunit L2;
R²⁶ and R²⁷ are each optional and are, independently, selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁—C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle; provided that both R²⁴ and R²⁵ are not H;
each R²⁹ is optional and independently selected from —C(O)—, —NH—, —C(O)—C₁-C₆ alkenylene-, —NH—C₁-C₆ alkenylene-, —C₁-C₆ alkenylene-NH—, —C₁-C₆ alkenylene-C(O)—, —NH(CO)NH—, and triazole;
the wavy line (˜) indicates the attachment site to R²⁰;
n20 is 1 to 26;
n21 is 1 to 4; and
n27 is 1 to 4.

In some embodiments, provided is a conjugate comprising a Linker, wherein both R²⁴ and R²⁵ of the PEG unit are not H. In some embodiments, provided is a Linker, wherein R²⁴ and R²⁵ of the PEG unit are each independently selected from H and polyhydroxyl group, provided that R²⁴ and R²⁵ are not both H.

In some embodiments, provided is a conjugate comprising a Linker, wherein the polyhydroxyl group is a linear monosaccharide, optionally selected from a C6 or C5 sugar, sugar acid or amino sugar. In some embodiments, provided is a conjugate comprising a Linker, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose talose, aldose, and ketose;
the sugar acid is selected from gluconic acid, aldonic acid, uronic acid and ulosonic acid; or the amino sugar is selected from glucosamine, N-acetyl glucosamine, galactosamine, and N-acetyl galactosamine.

In some embodiments, provided is a conjugate comprising a Linker, wherein the PEG unit is selected from the following, or a stereoisomer or salt thereof:

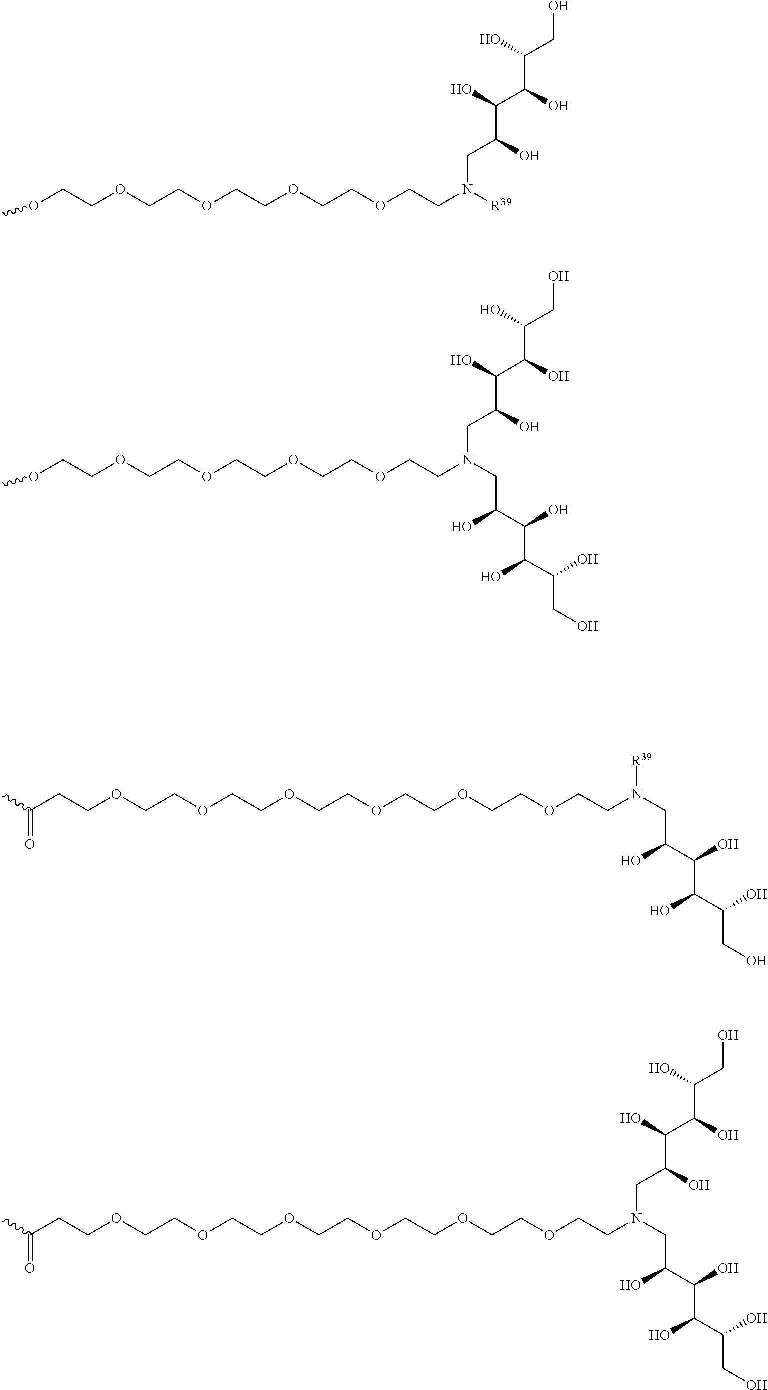

wherein R³⁹ is selected from H, a linear monosaccharide and polyethylene glycol, optionally having from 1 to 24 ethylene glycol subunits; and the wavy line at the left side indicates the attachment site to the subunit of the Amino Acid unit, the Stretcher unit and/or the portion of the Linker subunit.

In some embodiments, provided is a conjugate comprising a Linker, wherein one of R²⁴ and R²⁵ of the PEG unit is a linear monosaccharide and the other is a cyclic monosaccharide.

In some embodiments, provided is a conjugate comprising a Linker, wherein the PEG unit is selected from the following, or a stereoisomer or salt thereof:

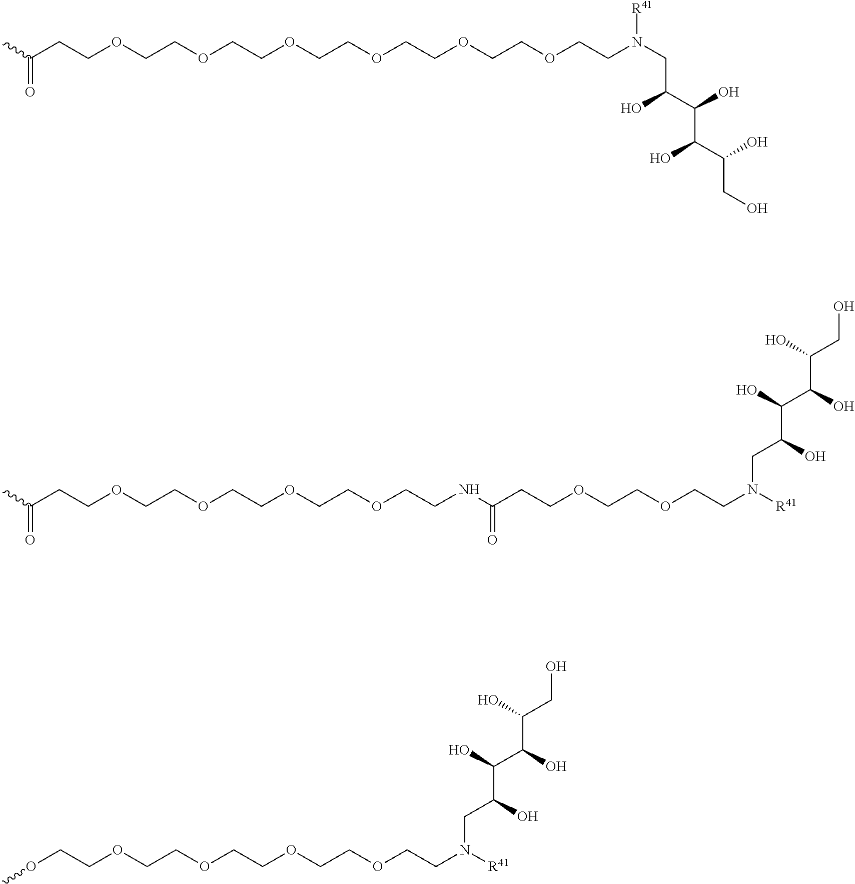

wherein R^{4′} is a cyclic monosaccharide; and the wavy line at the left side indicates the attachment site to the subunit of the Amino Acid unit, the Stretcher unit and/or the portion of the Linker subunit.

In some embodiments, provided is a conjugate comprising a Linker, wherein R²⁴ and R²⁵ of the PEG unit are independently selected from cyclic monosaccharides, disaccharides and polysaccharides. In some embodiments, provided is a conjugate comprising a Linker, wherein the PEG unit is selected from the following, or a stereoisomer or salt thereof:

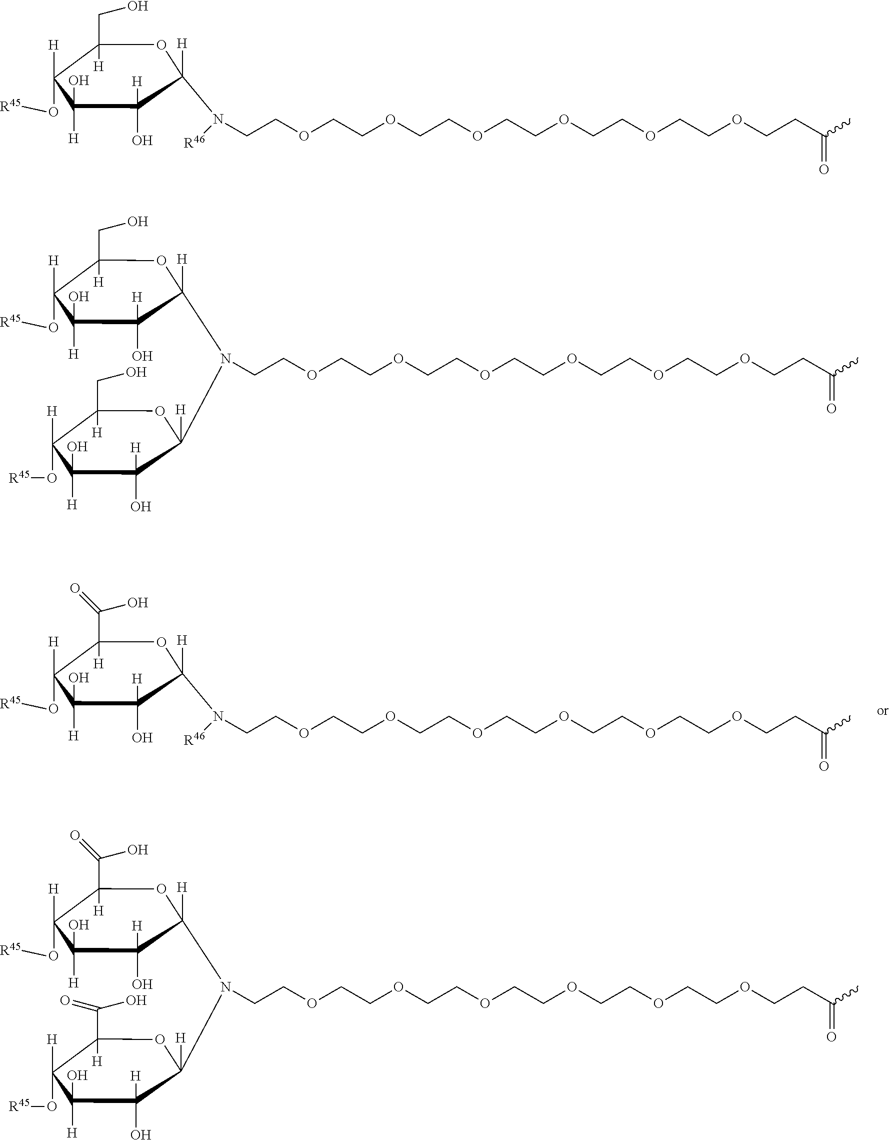

wherein each R⁴⁵ is selected from H and a monosaccharide, a disaccharide, or a polysaccharide; and R⁴⁶ is selected from a cyclic monosaccharide, disaccharide, or polysaccharide; and the wavy line at the right side indicates the attachment site to the subunit of the Amino Acid unit, the Stretcher unit and/or the portion of the Linker subunit.

In some embodiments, provided is a conjugate comprising a Linker, wherein R²⁴ and R²⁵ of the PEG unit are independently selected from a linear monosaccharide and a substituted linear monosaccharide, wherein the substituted linear monosaccharide is substituted with a monosaccharide, a disaccharide or a polysaccharide. In some embodiments, provided is a conjugate comprising a Linker, wherein the PEG unit is selected from the following, or a stereoisomer or salt thereof:

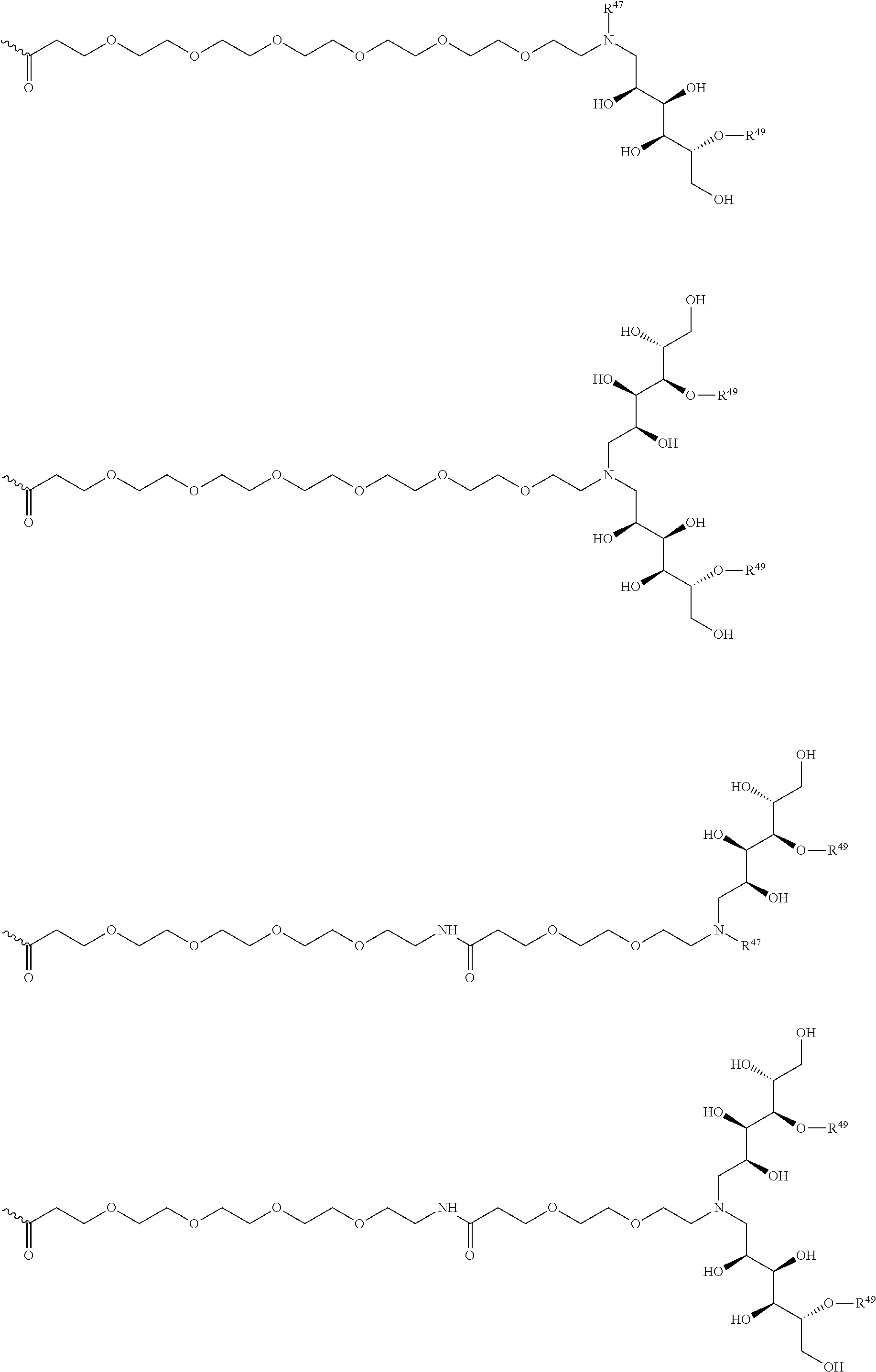

wherein R⁴⁷ is a linear monosaccharide; and each R⁴⁹ is selected from a monosaccharide, a disaccharide and a polysaccharide; and the wavy line at the left side indicates the attachment site to the subunit of the Amino Acid unit, the Stretcher unit and/or the portion of the Linker subunit.

In some embodiments, provided is a conjugate comprising a Linker, wherein R²⁴ and R²⁵ of the PEG unit are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide. In some embodiments, provided is a conjugate comprising a Linker, wherein the PEG unit is selected from the following, or a stereoisomer or salt thereof:

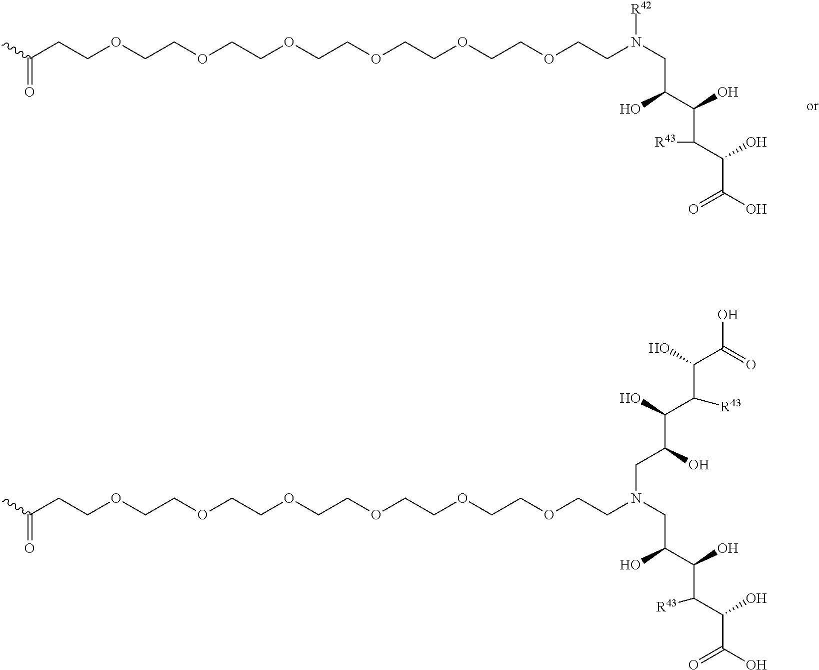

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line at the left side indicates the attachment site to the subunit of the Amino Acid unit, the Stretcher unit and/or the portion of the Linker subunit.

In some embodiments, provided is a conjugate comprising a Linker, wherein one of R²⁴ and R²⁵ of the PEG unit is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O— alkyl, aryl, carboxyl, ester, or amide. In some embodiments, provided is a conjugate comprising a Linker, wherein the PEG unit is selected from the following, or a stereoisomer or salt thereof:

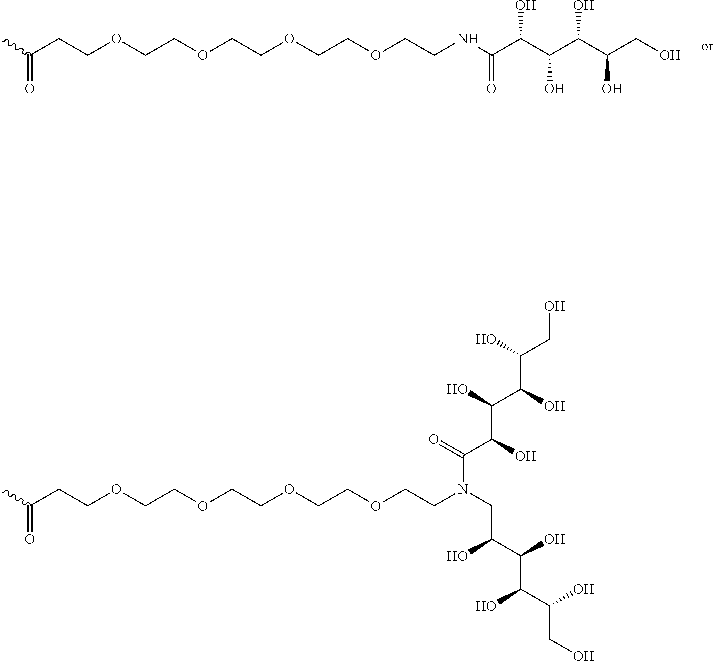

wherein the wavy line at the left side indicates the attachment site to the subunit of the Amino Acid unit, the Stretcher unit and/or the portion of the Linker subunit.

In some embodiments, provided is a conjugate comprising a Linker, wherein R²⁴ and R²⁵ of the PEG unit are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl and —C₁-C₃ alkyl are substituted with hydroxyl and/or carboxyl. In some embodiments, provided is a conjugate comprising a Linker, wherein the PEG unit is selected from the following, or a stereoisomer or salt thereof:

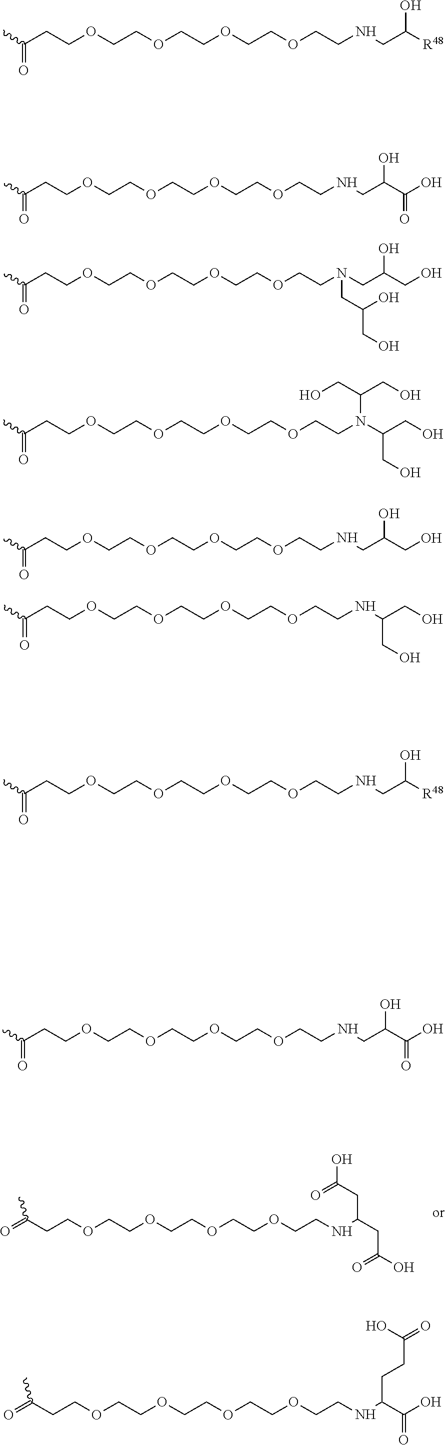

wherein R⁴⁸ is selected from H, OH, CH₂OH, COOH or —C₁-C₆ alkyl substituted with hydroxyl or carboxyl; and the wavy line at the left side indicates the attachment site to the subunit of the Amino Acid unit, the Stretcher unit and/or the portion of the Linker subunit.

In some embodiments, provided is a conjugate comprising a Linker, wherein one of R²⁴ and R²⁵ of the PEG unit is selected from H, substituted —C(O)—C₁-C₈ alkyl, substituted —C(O)—C₁-C₄ alkyl, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl. In some embodiments, provided is a conjugate comprising a Linker, wherein the PEG unit is selected from the following, or a stereoisomer or salt thereof:

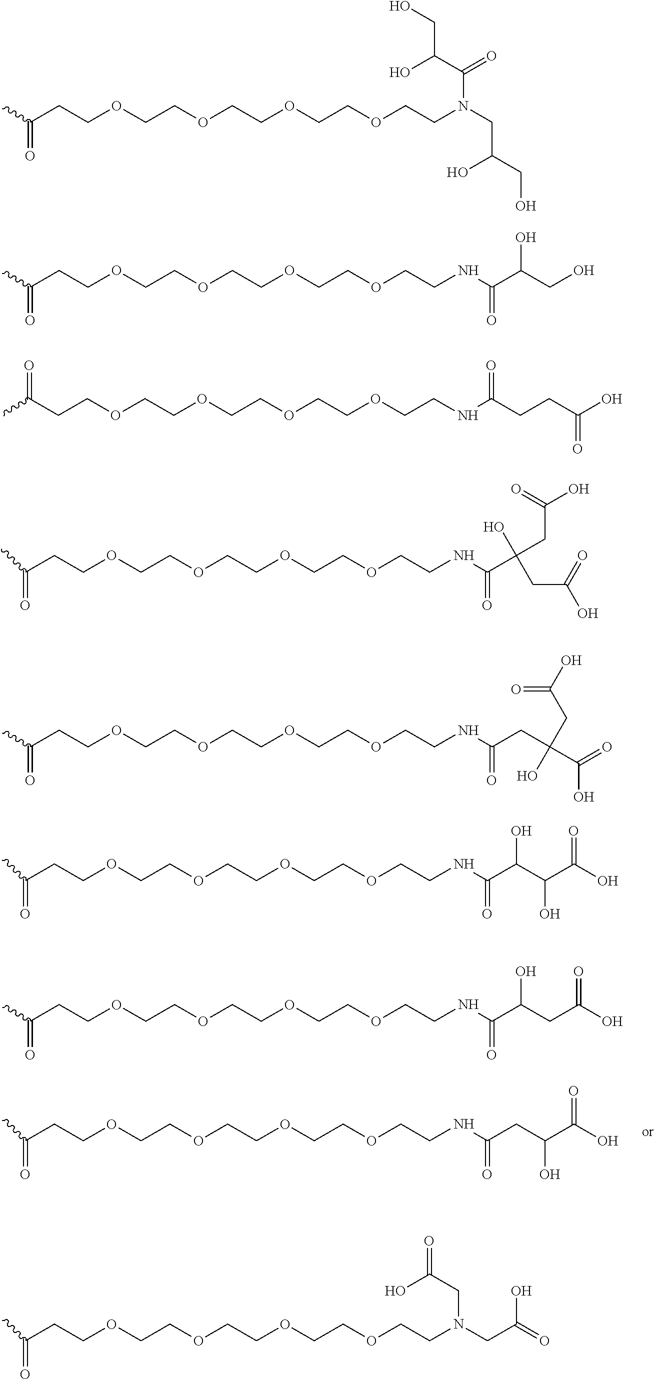

wherein the wavy line at the left side indicates the attachment site to the subunit of the Amino Acid unit, the Stretcher unit and/or the portion of the Linker subunit.

In some embodiments, provided is a conjugate comprising a Linker, wherein R²⁴ and R²¹ of the PEG unit are independently selected from H and a chelator, wherein the chelator is optionally attached to the nitrogen of —NR²⁴R²⁵ by an alkylene, arylene, carbocyclo, heteroarylene or heterocarbocylo; provided that both R²⁴ and R²⁵ are not H. In some embodiments, provided is a conjugate comprising a Linker, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine. In some embodiments, provided is a conjugate comprising a Linker, wherein the PEG unit is selected from the following, or a stereoisomer or salt thereof:

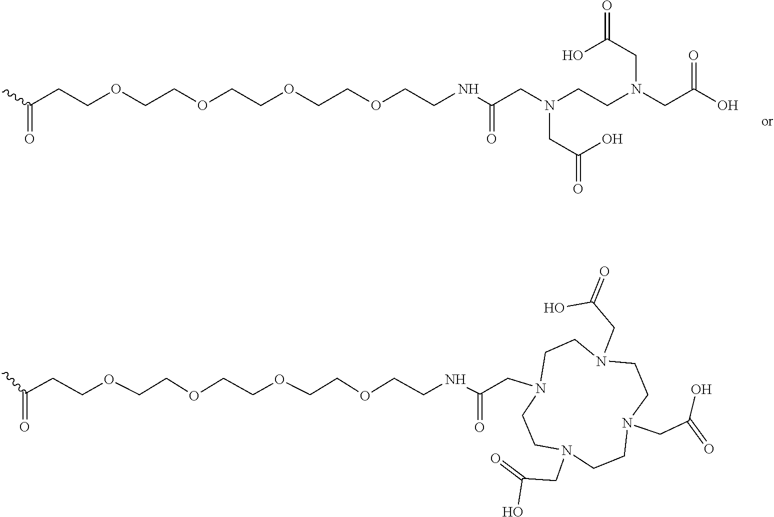

wherein the wavy line at the left side indicates the attachment site to the subunit of the Amino Acid unit, the Stretcher unit and/or the portion of the Linker subunit.

In some embodiments, provided is a conjugate comprising a Linker, wherein each monosaccharide of a Sugar unit or a PEG unit is independently selected from:
a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine; a sugar acid selected from gluconic acid, aldonic acid, uronic acid and ulosonic acid; or an amino sugar is selected from glucosamine, N-acetyl glucosamine, galactosamine, and N-acetyl galactosamine.

In some embodiments, provided is a conjugate comprising a Linker, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.

In some embodiments, provided is a conjugate comprising a Linker, wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, provided is a conjugate comprising a Linker, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.

In some embodiments, provided is a conjugate comprising a Linker, wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, the Linker comprises a PEG unit having a formula selected from:

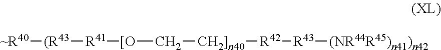

or a salt thereof, wherein:
R⁴⁰ is a functional group for attachment to a subunit of the Amino Acid unit, the Stretcher unit and/or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
provided that both R⁴⁴ and R⁴⁵ are not H;
the wavy line (˜) indicates the attachment site to R⁴⁰;
n40 is 1 to 26;
n41 is 1 to 6; and
n42 is 1 to 6.

In some embodiments, the Linker comprises a PEG unit having a formula selected from:

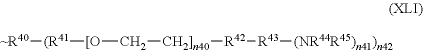

or a salt thereof, wherein:
R⁴⁰ is a functional group for attachment to a subunit of the Amino Acid unit, the Stretcher unit and/or a portion of the Linker Subunit L2;
R⁴¹ and R⁴² are absent or are each, independently, C₁-C₆ alkylene;
R⁴³ is absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
provided that both R⁴⁴ and R⁴⁵ are not H;
the wavy line (˜) indicates the attachment site to R⁴⁰;
n40 is 1 to 26;
n41 is 1 to 6; and
n42 is 1 to 6.

In some embodiments, the Linker comprises a PEG unit having a formula selected from:

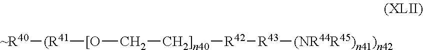

or a salt thereof, wherein:
R⁴⁰ is a functional group for attachment to a subunit of the Amino Acid unit, the Stretcher unit and/or a portion of the Linker Subunit L2;
R⁴¹ and R⁴² are absent or are each, independently, C₁-C₃ alkylene;
R⁴³ is absent or is selected from selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
provided that both R⁴⁴ and R⁴⁵ are not H;
the wavy line (˜) indicates the attachment site to R⁴⁰;
n40 is 1 to 26;
n41 is 1 to 4; and
n42 is 1 to 4.

In some embodiments, provided is a conjugate comprising a Linker, wherein R⁴⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkyl heteroaryl, or protected forms thereof.

In some embodiments, provided is a conjugate comprising a Linker, wherein R²⁰ or R⁴⁰ has one of the following structures:

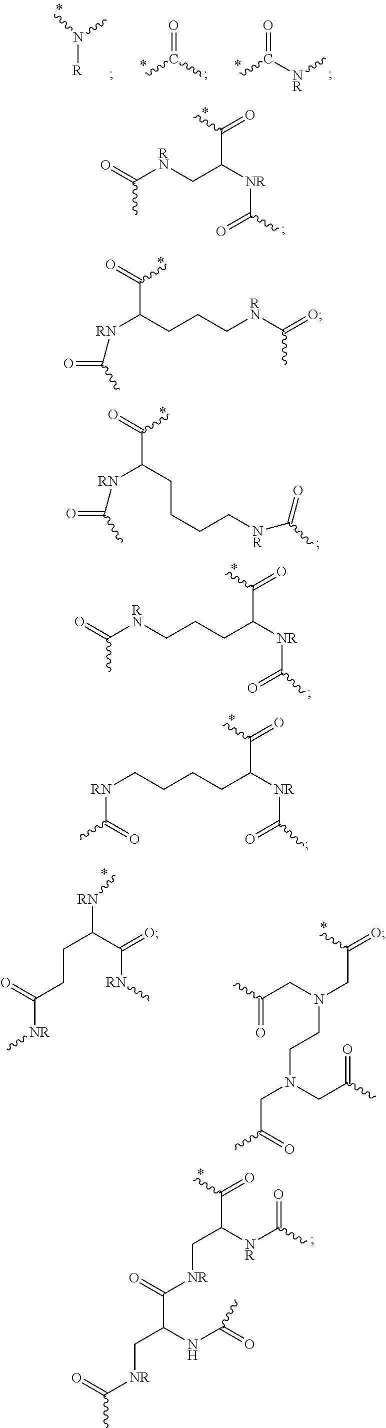

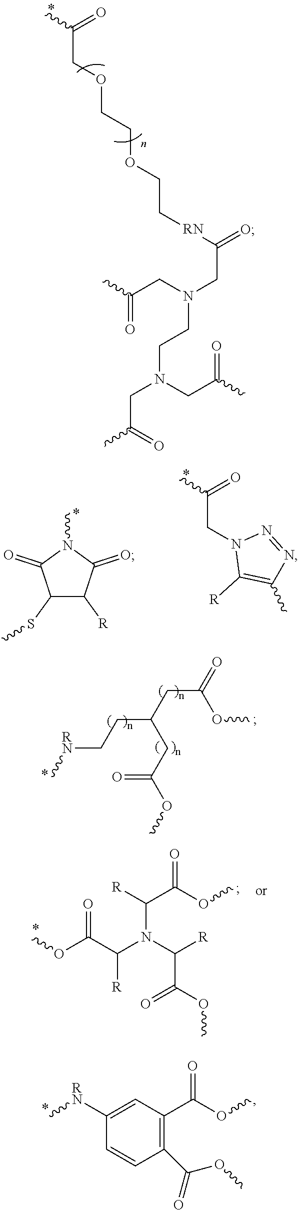

wherein R═H or C₁₋₆alkyl; and n=0 to 12 or a stereoisomer thereof, wherein the (*) indicates the attachment site of R²⁰ or R⁴⁰ to a subunit of the Amino Acid unit, the Stretcher unit and/or a portion of the Linker Subunit L2 and the ( [symbol] ) indicates the attachment site of R²⁰ or R⁴⁰ to the remainder of the PEG unit.

In some embodiments, provided is a conjugate comprising a Linker, wherein R²⁰ or R⁴⁰ has one of the following structures:

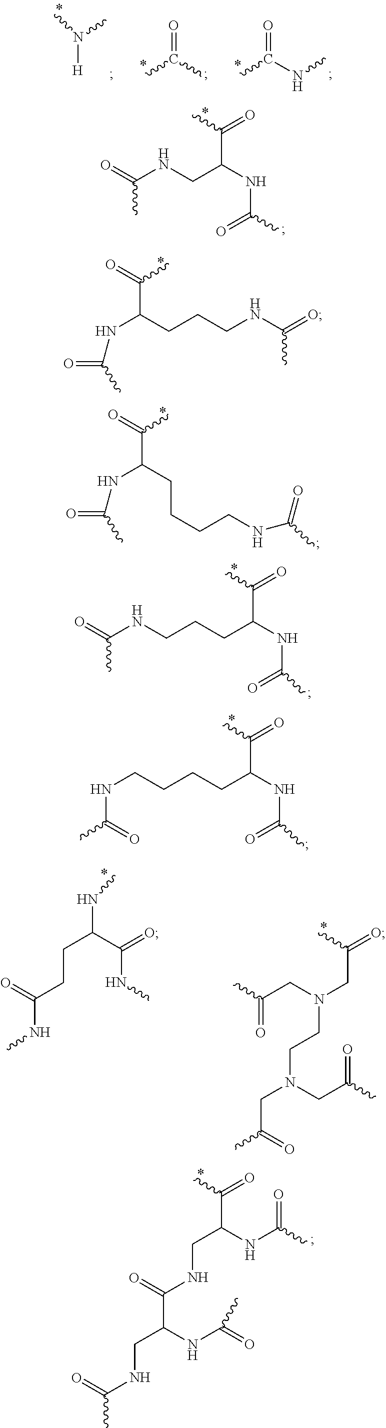

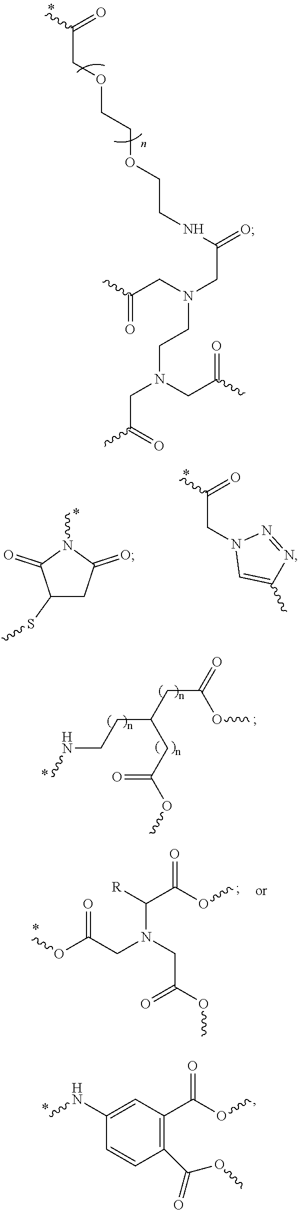

wherein n=0 to 12 or a stereoisomer thereof, wherein the (*) indicates the attachment site of R²⁰ or R⁴⁰ to a subunit of the Amino Acid unit, the Stretcher unit and/or a portion of the Linker Subunit L2 and the ( [symbol] ) indicates the attachment site of R²⁰ or R⁴⁰ to the remainder of the PEG unit.

In some embodiments, provided is a conjugate comprising a Linker, wherein R⁴³—(NR⁴⁵R⁴⁵)ₙ₄₁, when R⁴³ is present, has one of the following structures:

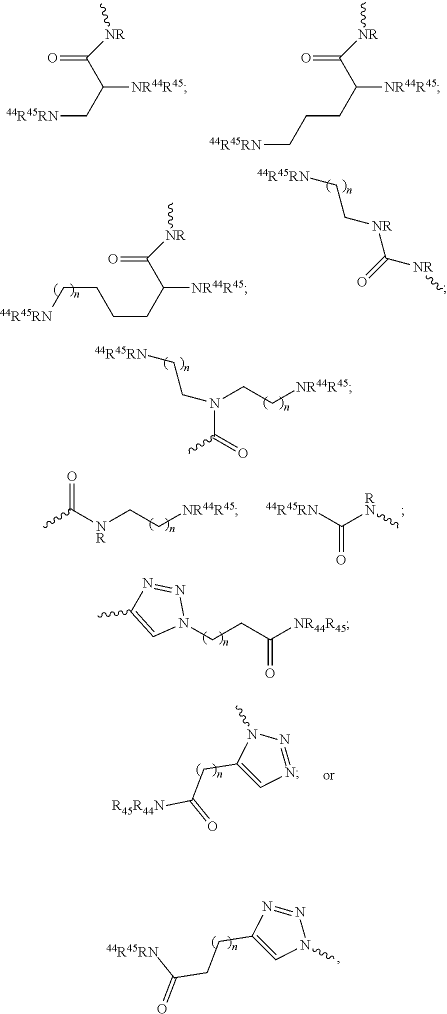

wherein n is 0-6 and R═H, C₁₋₆alkyl, polyhydroxyl, or substituted polyhydroxyl
or a stereoisomer thereof, wherein the ( [symbol] ) indicates the attachment site of R⁴³ to the remainder of the PEG unit.

In some embodiments, provided is a conjugate comprising a Linker, wherein R⁴³—(NR⁴⁴R⁴⁵)ₙ₄₁, when R⁴³ is present, has one of the following structures:

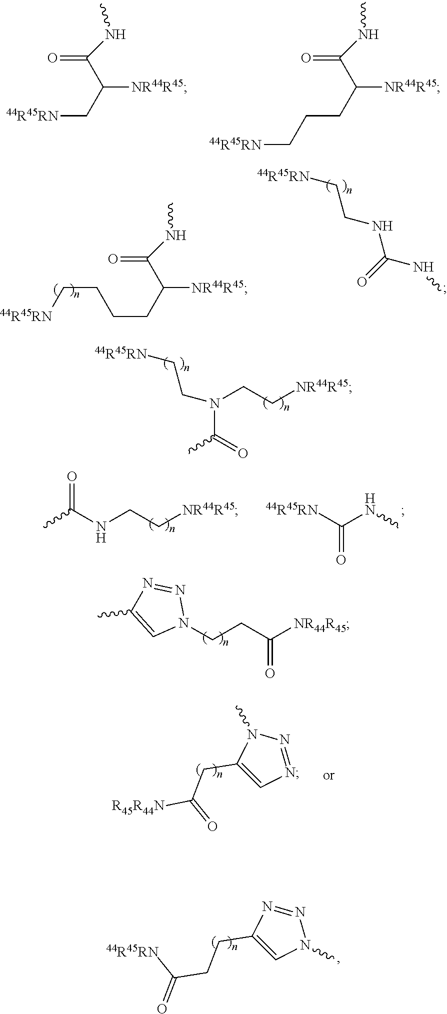

or a stereoisomer thereof, wherein the ( [symbol] ) indicates the attachment site of R⁴³ to the remainder of the PEG unit.

In some embodiments, provided is a conjugate comprising a Linker, wherein —NR⁴⁴R⁴⁵ has one of the following structures:

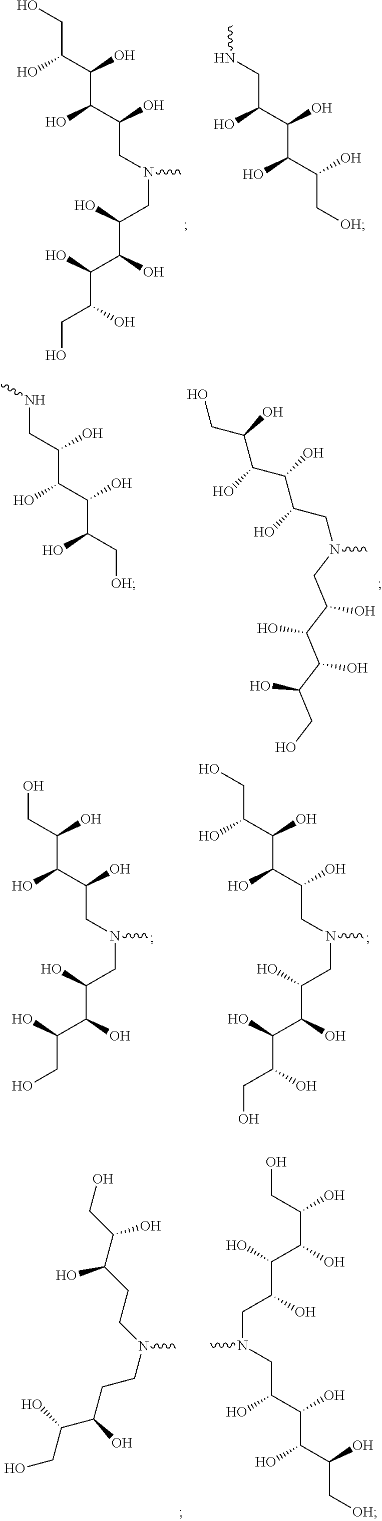

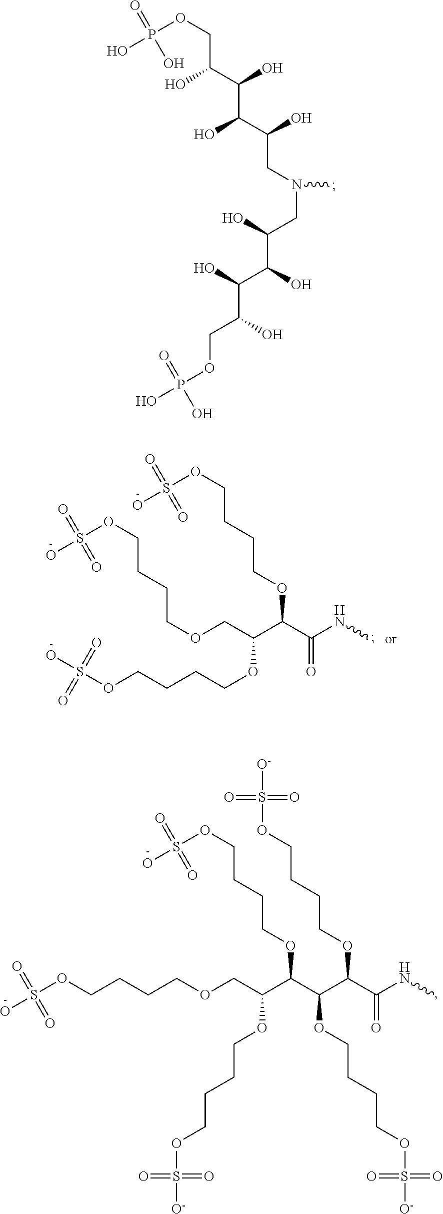

or a stereoisomer thereof, wherein the ( [symbol] ) indicates the attachment site of —NR⁴⁴R⁴⁵ to the remainder of the PEG unit.

In some embodiments, provided is a conjugate comprising a Linker, wherein the PEG unit has one of the following structures, or a stereoisomer thereof, prior to attachment to the Amino Acid unit, the Stretcher unit and/or to a portion of the Linker Subunit L2:

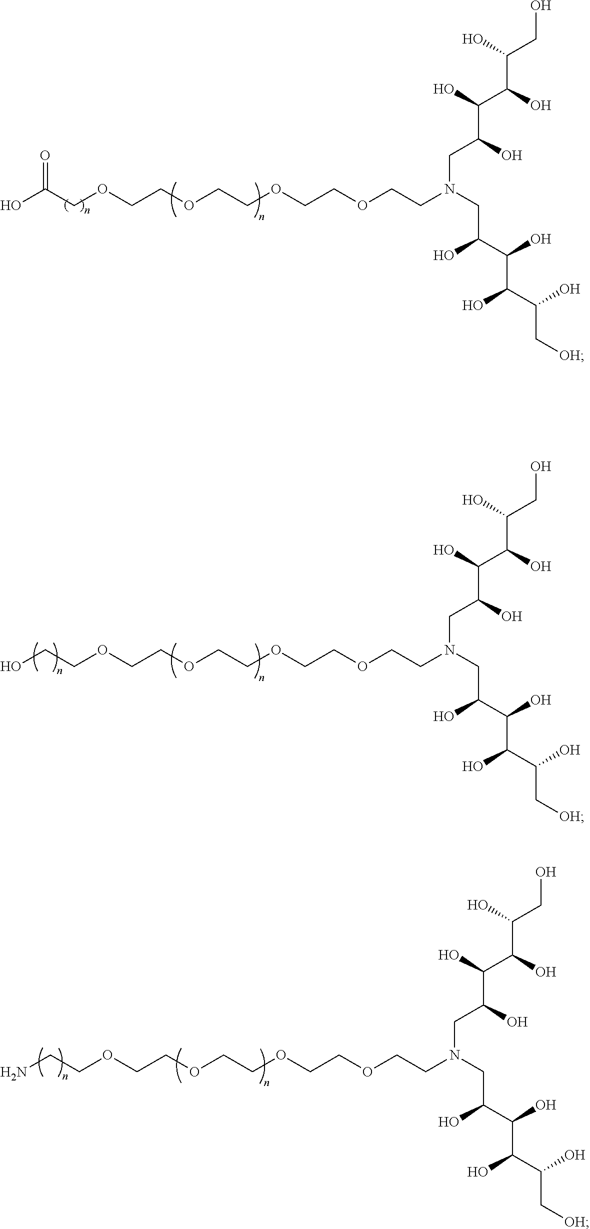

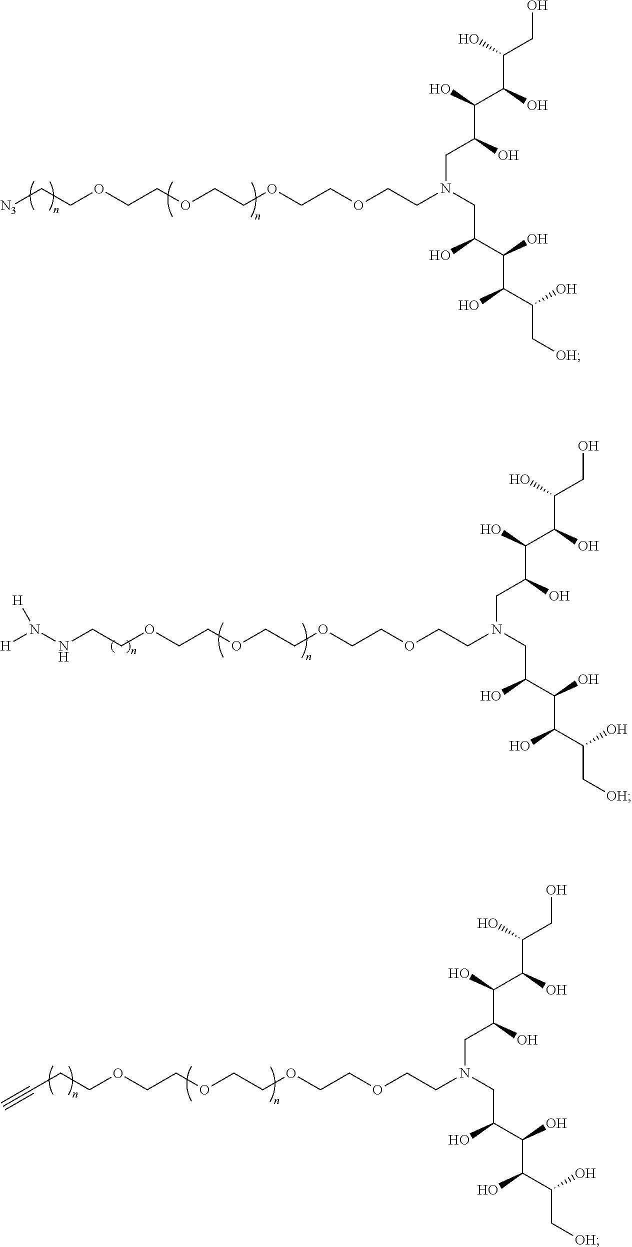

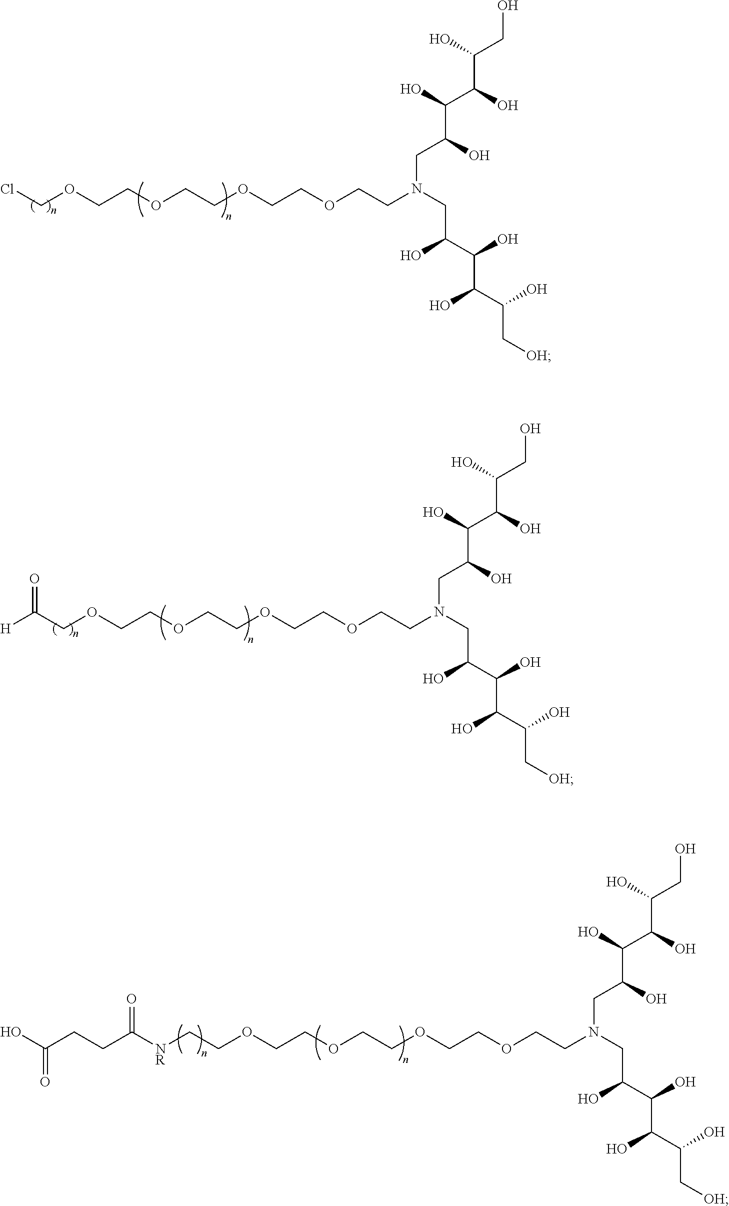

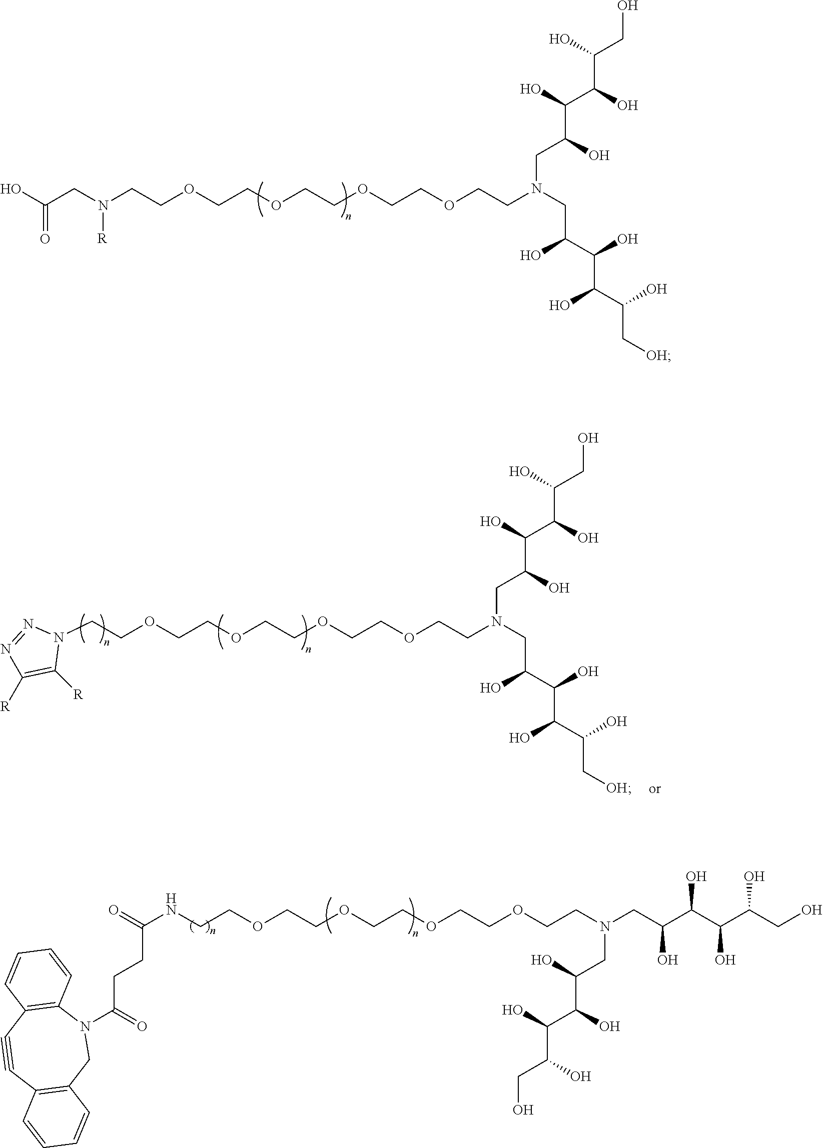

wherein R is H or alkyl, and each n is individually 1 to 12, and wherein the functional group moiety of the attachment site of the PEG unit may be selected from carboxyl, hydroxyl, aminyl, azidyl, hydrazinyl, alkynyl, formyl, or triazolyl as depicted above.

In some embodiments, the Linker comprises a PEG unit having a formula selected from:

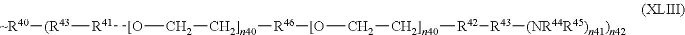

or a salt thereof, wherein:
R⁴⁰ is a functional group for attachment to a subunit of the Amino Acid unit, the Stretcher unit and/or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
R⁴⁶ is selected from amino, amino-alkyl-amino, or —NH—C(O)—NH—S(O)₂—NH—; the wavy line (˜) indicates the attachment site to R⁴⁰;
n40 is 1 to 26;
n41 is 1 to 6; and
n42 is 1 to 6.

In some embodiments, provided is a conjugate comprising a Linker, wherein the PEG unit has one of the following structures prior to attachment to the Amino Acid unit, the Stretcher unit and/or to a portion of the Linker Subunit L2:

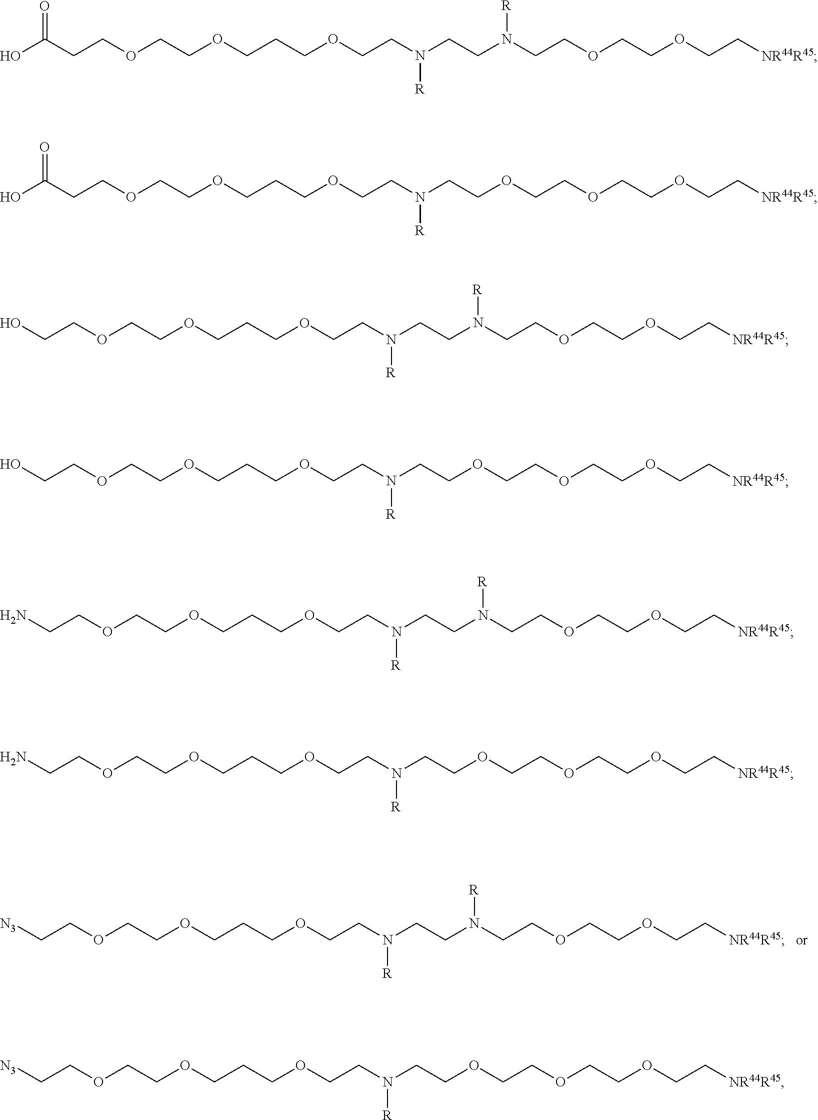

wherein R is H or alkyl, and n is 1 to 12, and wherein the functional group moiety of the attachment site of the PEG unit may be selected from carboxyl, hydroxyl, aminyl or azidyl.

In some embodiments, the Linker comprises a PEG unit having a formula selected from:

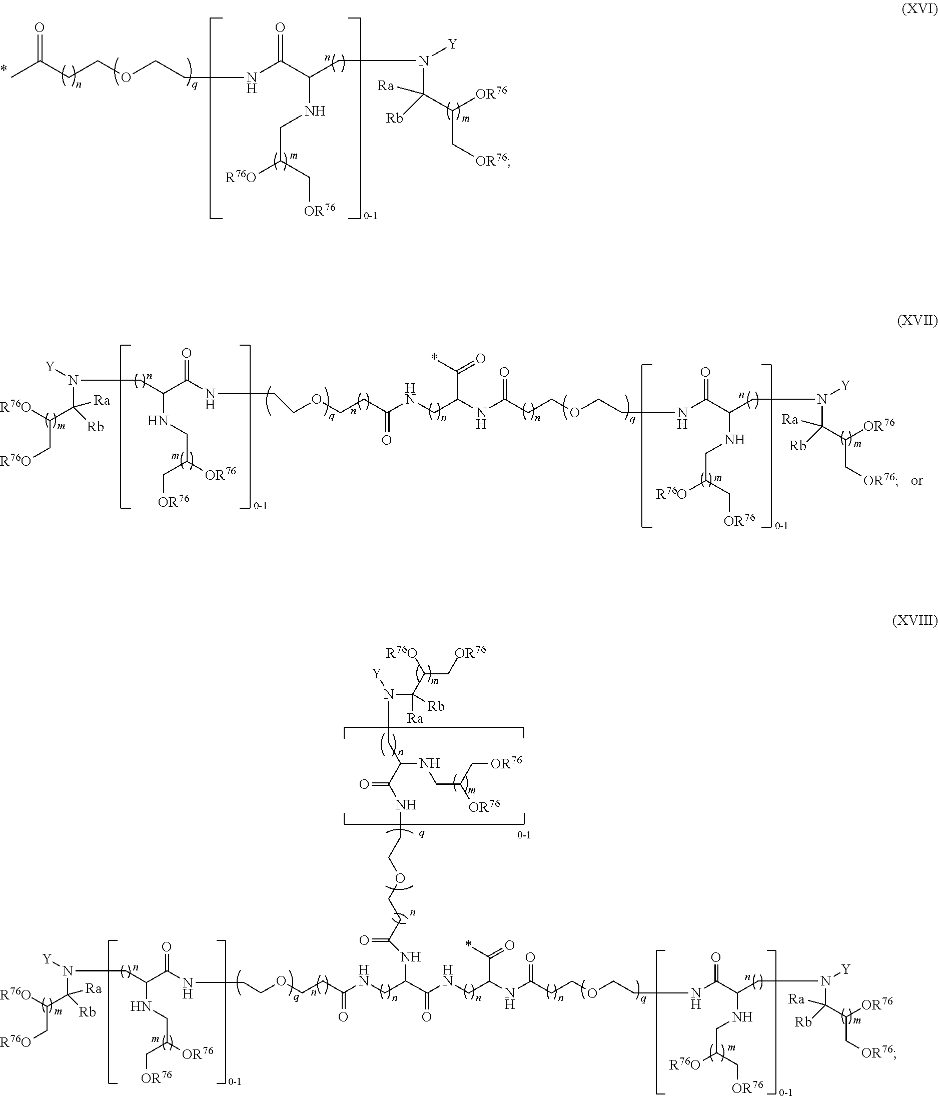

or a stereoisomer or salt thereof, wherein:
   each Y is independently R⁷⁶ or

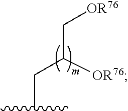

each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

In some embodiments, the Linker comprises a PEG unit having a formula selected from:

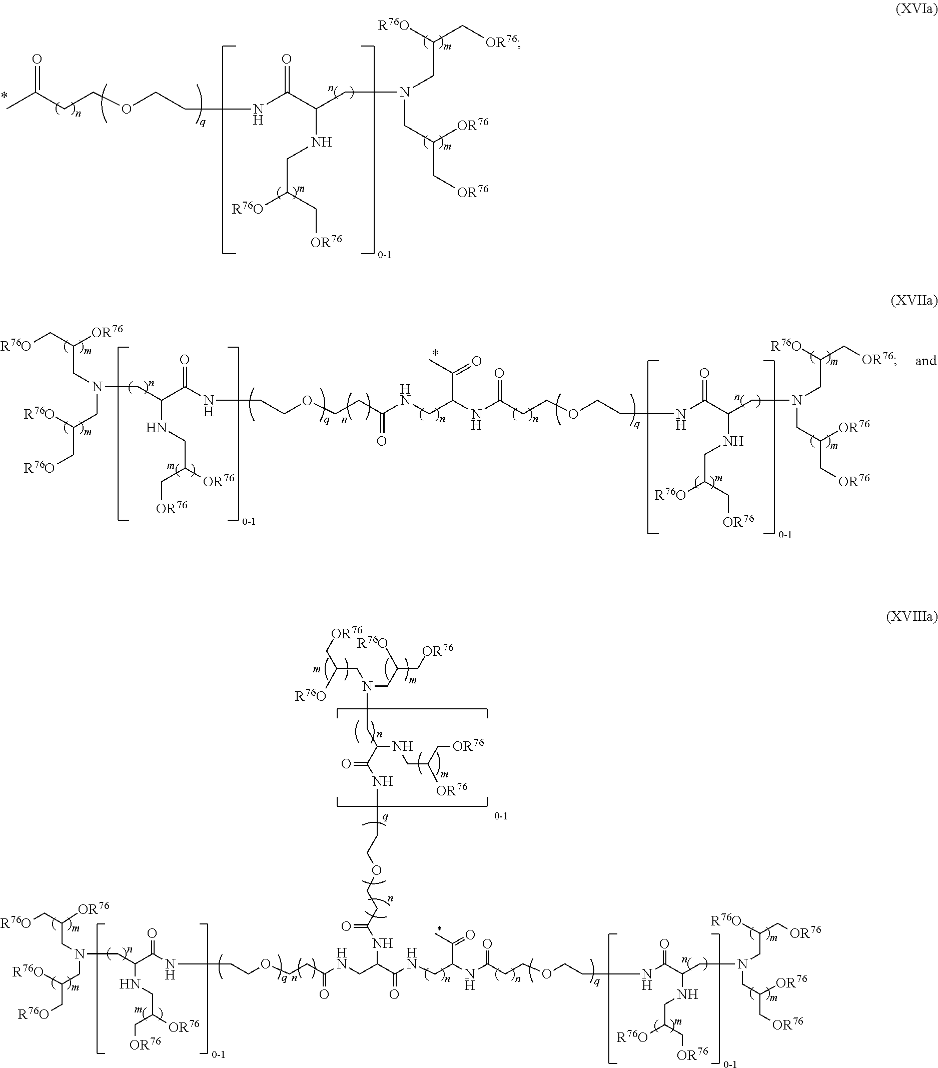

or a stereoisomer or salt thereof, wherein:
   each R⁷⁶ is independently H, acetyl, —P(═O)(OH)₂, or —(CH₂)ᵥS(═O)₂(OH);
   each q is independently 1-26;
   each m is independently 1 to 4;
   each n is independently 1 to 4;
   each v is independently 1 to 6; and
   each * indicates an attachment site for a subunit of the Amino Acid unit (AA), the Linker subunit L2, or the Stretcher unit (L1).

In some embodiments, the Linker comprises a PEG unit having a formula selected from:

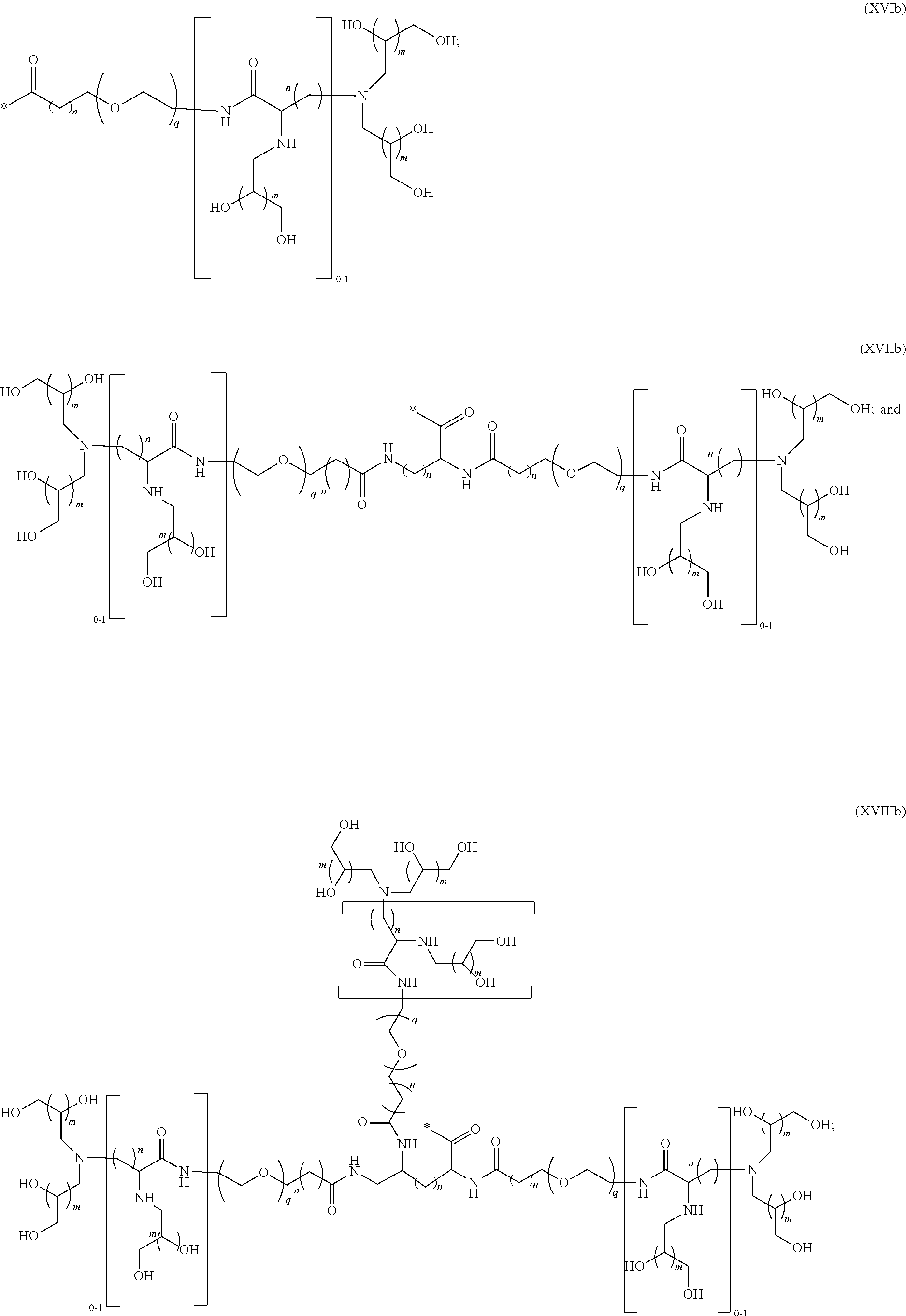

or a stereoisomer or salt thereof, wherein:
   each q is independently 1-26;
   each m is independently 1 to 4;
   each n is independently 1 to 4; and
   each * indicates an attachment site for a subunit of the Amino Acid unit (AA), the Linker subunit L2, or the Stretcher unit (L1).

In some embodiments, provided is a conjugate comprising a Linker, wherein Y is R⁷⁶.

In some embodiments, provided is a conjugate comprising a Linker, wherein Y is

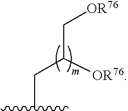

In some embodiments, provided is a conjugate comprising a Linker, wherein each Rₐ and R_{b} is independently H.

In some embodiments, provided is a conjugate comprising a Linker, wherein Rₐ and R_{b} are taken together with the carbon to which they are attached to form an oxo group.

In some embodiments, provided is a conjugate comprising a Linker, wherein q is 10-20.

In some embodiments, provided is a conjugate comprising a Linker, wherein q is 12.

### Carboxyl Units

In some embodiments, the Linker comprises a Carboxyl unit having the following formula:

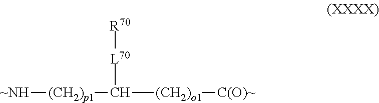

or a salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
R⁷⁰ is —NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is absent or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
   each wavy line (˜) indicates an attachment site for another subunit of an Amino Acid unit (AA), the Linker subunit L2, or the Stretcher unit (L1); and
   each of p1 and o1 are independently selected from 0 to 2;
   or
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is —NR⁷¹(R⁷⁵_(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
   each wavy line (˜) indicates an attachment site for another subunit of an Amino Acid unit (AA), the Linker subunit L2, or the Stretcher unit (L1); and
   each of p1 and o1 are independently selected from 0 to 2;
   or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is ˜N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
   each wavy line (˜) indicates an attachment site for another subunit of an Amino Acid unit (AA), the Linker subunit L2, or the Stretcher unit (L1); and
   each of p1 and o1 are independently selected from 0 to 2.

In some embodiments, provided is a conjugate comprising a Linker comprising at least one Sugar unit. In some embodiments, provided is a conjugate comprising a Linker comprising at least one PEG unit. In some embodiments, provided is a conjugate comprising a Linker comprising at least one Carboxyl unit. In some embodiments, provided is a conjugate comprising a Linker comprising at least two Polar units, each Polar unit selected from a Sugar unit, a PEG unit and a Carboxyl unit. In some embodiments, provided is a conjugate comprising a Linker comprising at least one Sugar unit and a PEG unit or a Carboxyl unit. In some embodiments, provided is a conjugate comprising a Linker comprising at least one Carboxyl unit and a PEG unit.

In some embodiments, provided is a conjugate comprising a Linker, wherein the Amino Acid unit (AA) is present (s=1). In some embodiments, provided is a conjugate comprising a Linker, wherein the Amino Acid unit comprises at least one Polar unit.

In some embodiments, provided is a conjugate comprising a Linker, wherein L2 or AA-L2 has one of the following structures, or a stereoisomer thereof:

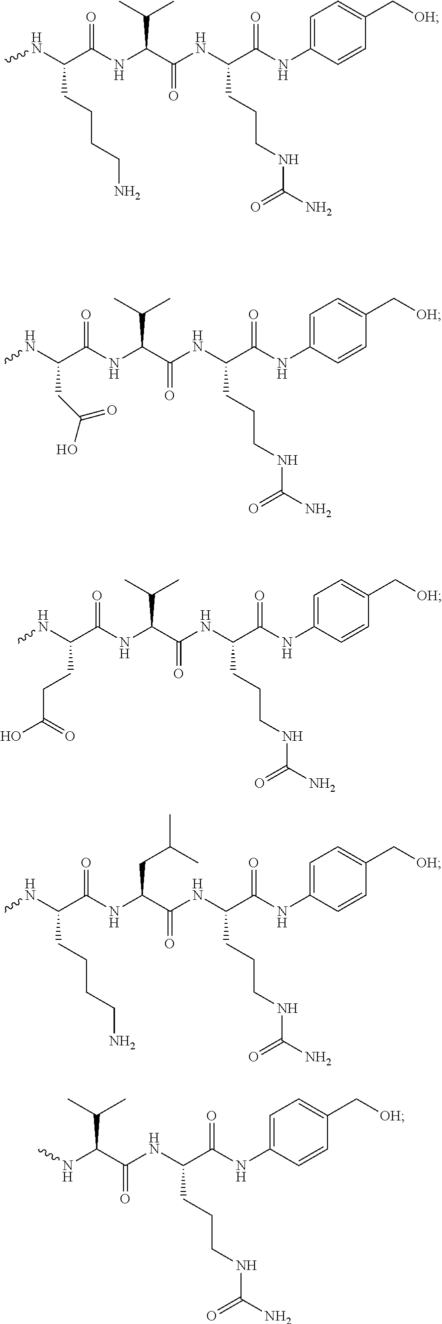

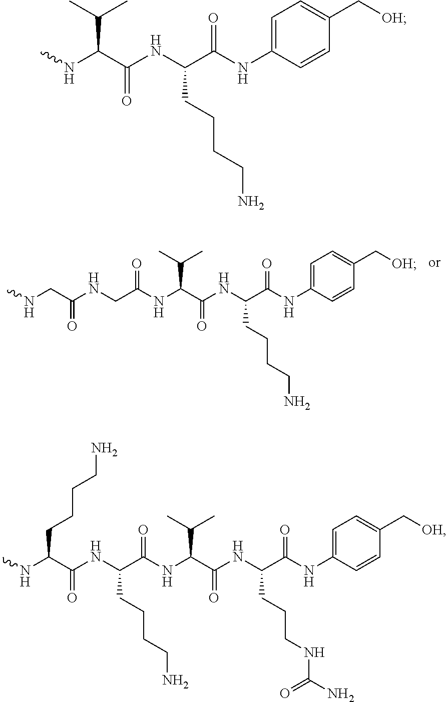

wherein the wavy line on the amino group indicates an attachment site for a Stretcher unit or an Amino Acid unit, and the Drug unit is attached to the benzyl alcohol.

In some embodiments, provided is a conjugate comprising a Linker, wherein the Linker comprises ˜AA-L2˜ having a formula selected from the following:

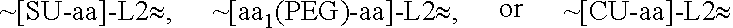

wherein the square brackets indicate the Amino Acid unit, each aa is an optional subunit of AA, L2 is the Linker Subunit, each wavy line (˜) indicates an attachment site for a Stretcher unit; aa₁(PEG) is a PEG unit attached to an amino acid subunit of AA, SU is a Sugar unit attached to a subunit of AA or to L2, and CU is a Carboxyl unit attached to a subunit of AA or to L2; and the double wavy (≈) line indicates an attachment site for a Drug unit, wherein aa and aa₁ are independently selected from alpha, beta and gamma amino acids and derivatives thereof.

In some embodiments, provided is a conjugate comprising a Linker, wherein the Linker comprises ˜AA-L2˜ having a formula selected from the following:

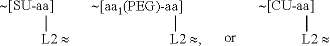

wherein the square brackets indicate the Amino Acid unit, each aa is an amino acid subunit of AA, L2 is the Linker Subunit attached to a side chain of aa, the wavy line (˜) indicates an attachment site for a Stretcher unit; aa₁(PEG) is a PEG unit attached to aa, SU is a Sugar unit attached to aa, CU is a Carboxyl unit attached to aa, and the double wavy (≈) line indicates an attachment site for a Drug unit; wherein aa and aa₁ are independently selected from alpha, beta and gamma amino acids and derivatives thereof.

In some embodiments, provided is a conjugate comprising a Linker, wherein the Amino Acid unit comprises at least two Polar units.

In some embodiments, provided is a conjugate comprising a Linker, wherein the Linker comprises ˜AA-L2˜ having a formula selected from the following:

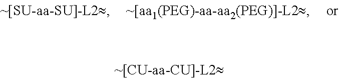

wherein the square brackets indicate the Amino Acid unit, aa is an optional subunit of AA, L2 is the Linker Subunit, the wavy line (˜) indicates an attachment site for a Stretcher unit; each of aa₁(PEG) and aa₂(PEG) is a PEG unit attached to aa or to the other PEG unit; each SU is a Sugar unit attached to aa or the other Sugar unit, each CU is a Carboxyl unit attached to aa or to the other Carboxyl unit, and the double wavy (≈) line indicates an attachment site for a Drug unit; wherein aa, aa₁ and aa₂ are independently selected from selected from alpha, beta and gamma amino acids and derivatives thereof.

In some embodiments, provided is a conjugate comprising a Linker, wherein the Linker comprises ˜AA-L2˜ having a formula selected from the following:

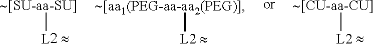

wherein the square brackets indicate the Amino Acid unit, aa is an amino acid subunit of AA, L2 is a Linker Subunit attached to a side chain of aa, each wavy line (˜) indicates an attachment site for a Stretcher unit; each of aa₁(PEG) and aa₂(PEG) is a PEG unit attached to aa, each SU is a Sugar unit attached to aa; each CU is a Carboxyl unit attached to aa; and the double wavy (≈) line indicates an attachment site for a Drug unit; wherein each of aa, aa₁ and aa₂ is independently selected from alpha, beta and gamma amino acids and derivatives thereof.

In some embodiments, provided is a conjugate comprising a Linker, wherein Linker Subunit L2 is a cleavable linker unit. In some embodiments, provided is a conjugate comprising a Linker, wherein Linker Subunit L2 comprises a peptide that is cleavable by an intracellular protease. In some embodiments, provided is a conjugate comprising a Linker, wherein the cleavable peptide comprises a valine-citrulline peptide, a valine-alanine peptide, a valine-lysine peptide, a phenylalanine-lysine peptide, or a glycine-glycine-phenylalanine-glycine peptide.

In some embodiments, provided is a conjugate comprising a Linker, wherein Linker Subunit L2 comprises at least one Polar unit. In some embodiments, provided is a conjugate comprising a Linker, wherein the Polar unit is a Sugar unit (SU). In some embodiments, provided is a conjugate comprising a Linker, wherein the cleavable peptide comprises a SU-valine-citrulline peptide, a SU-valine-lysine peptide, a SU-valine-alanine peptide, a SU-phenylalanine-lysine peptide, or a SU-glycine-glycine-phenylalanine-glycine peptide.

In some embodiments, provided is a conjugate comprising a Linker, wherein the Polar unit is a Carboxyl unit (CU). In some embodiments, provided is a conjugate comprising a Linker, wherein the cleavable peptide comprises a CU-valine-citrulline peptide, a CU-valine-lysine peptide, a valine-(CU-lysine) peptide, a CU-valine-alanine peptide, a CU-phenylalanine-lysine peptide, a phenylalanine-(CU-lysine) peptide or a CU-glycine-glycine-phenylalanine-glycine peptide, wherein CU-lysine is a Carboxyl unit comprising a lysine residue.

In some embodiments, provided is a conjugate comprising a Linker, wherein the Polar unit is a PEG unit (PEG). In some embodiments, provided is a conjugate comprising a Linker, wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG)) peptide or a Lys(PEG)-glycine-glycine-phenylalanine-glycine peptide, wherein Lys(PEG) and Cit(PEG) comprise a PEG unit attached to a lysine residue or a citrulline residue, respectively.

In some embodiments, provided is a conjugate comprising a Linker, wherein the cleavable peptide is attached to para-aminobenzyl alcohol self immolative group (PABA).

In some embodiments, provided is a conjugate comprising a Linker, wherein the Amino Acid unit is joined to Linker Subunit L2 by a non-peptidic linking group. In some embodiments, provided is a conjugate comprising a Linker, wherein the non-peptidic linking group is selected from C₁-C₁₀ alkylene, C₂-C₁₀ alkenylene, C₂-C₁₀ alkynylene, or polyethylene glycol.

In some embodiments, provided is a conjugate comprising a Linker, further comprising a Stretcher unit. In some embodiments, provided is a conjugate comprising a Linker, wherein the Stretcher unit is selected from the following:

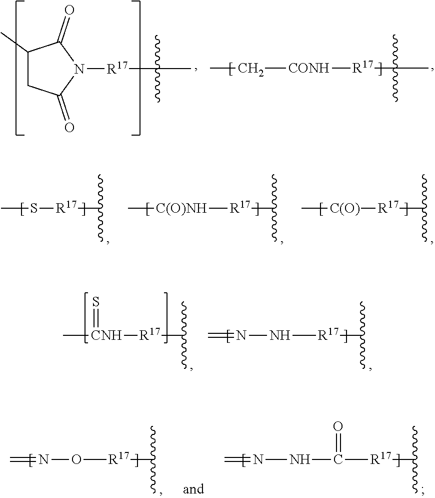

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or wherein the Stretcher unit comprises maleimido(C₁-C₁₀alkylene-C(O)—, maleimido(CH₂OCH₂)ₚ₂(C₁-C₁₀alkylene)C(O)—, maleimido(C₁-C₁₀alkylene)(CH₂OCH₂)ₚ₂C(O)—, or a ring open form thereof, wherein p2 is from 1 to 26.

In some embodiments, provided is a conjugate comprising a Linker, wherein the Stretcher unit is selected from the following:

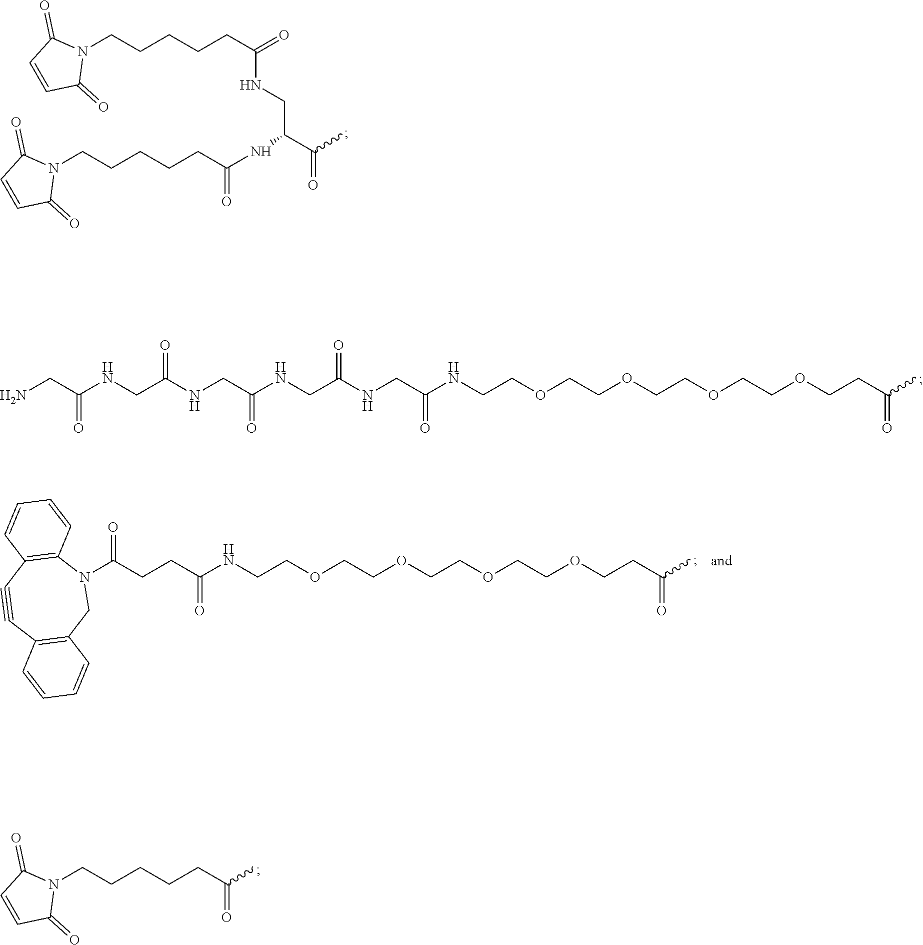

wherein the wavy line [symbol] indicates an attachment site of the Stretcher unit to an Amino Acid unit or to a Linker Subunit L2, and the attachment site to the Binding unit is on a maleimide, primary amine or alkyne functional group.

In some embodiments, provided is a conjugate comprising a Linker having one of the following structures, or a stereoisomer thereof:

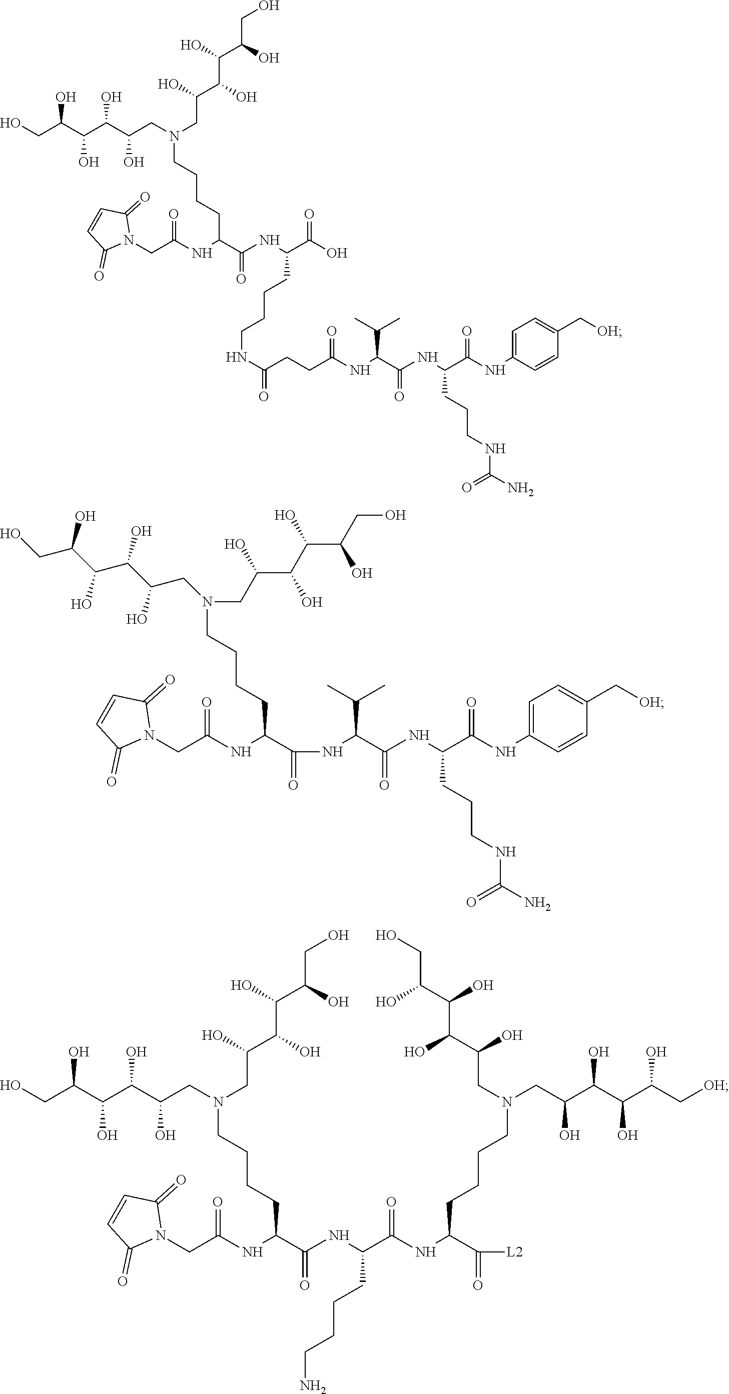

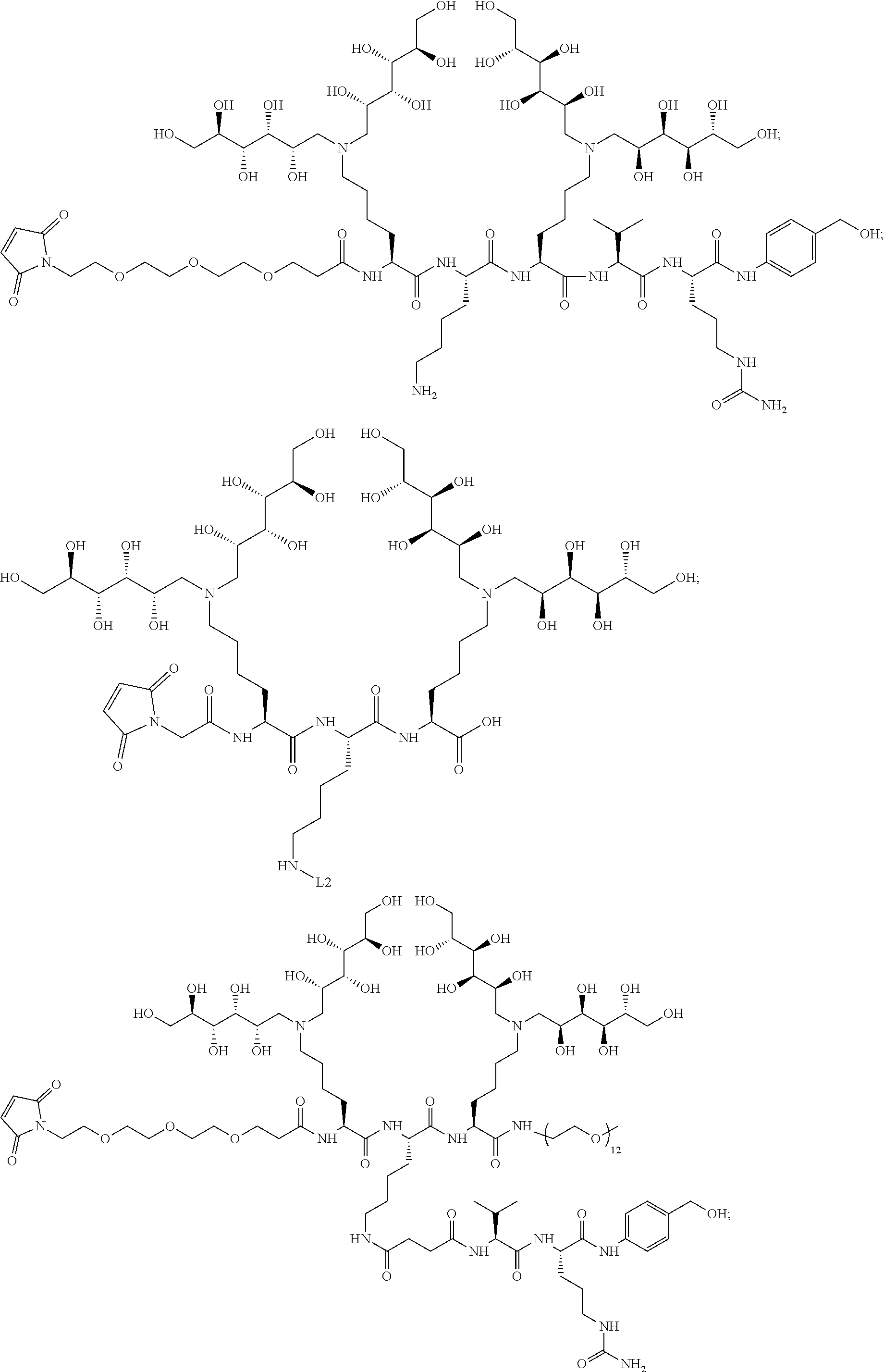

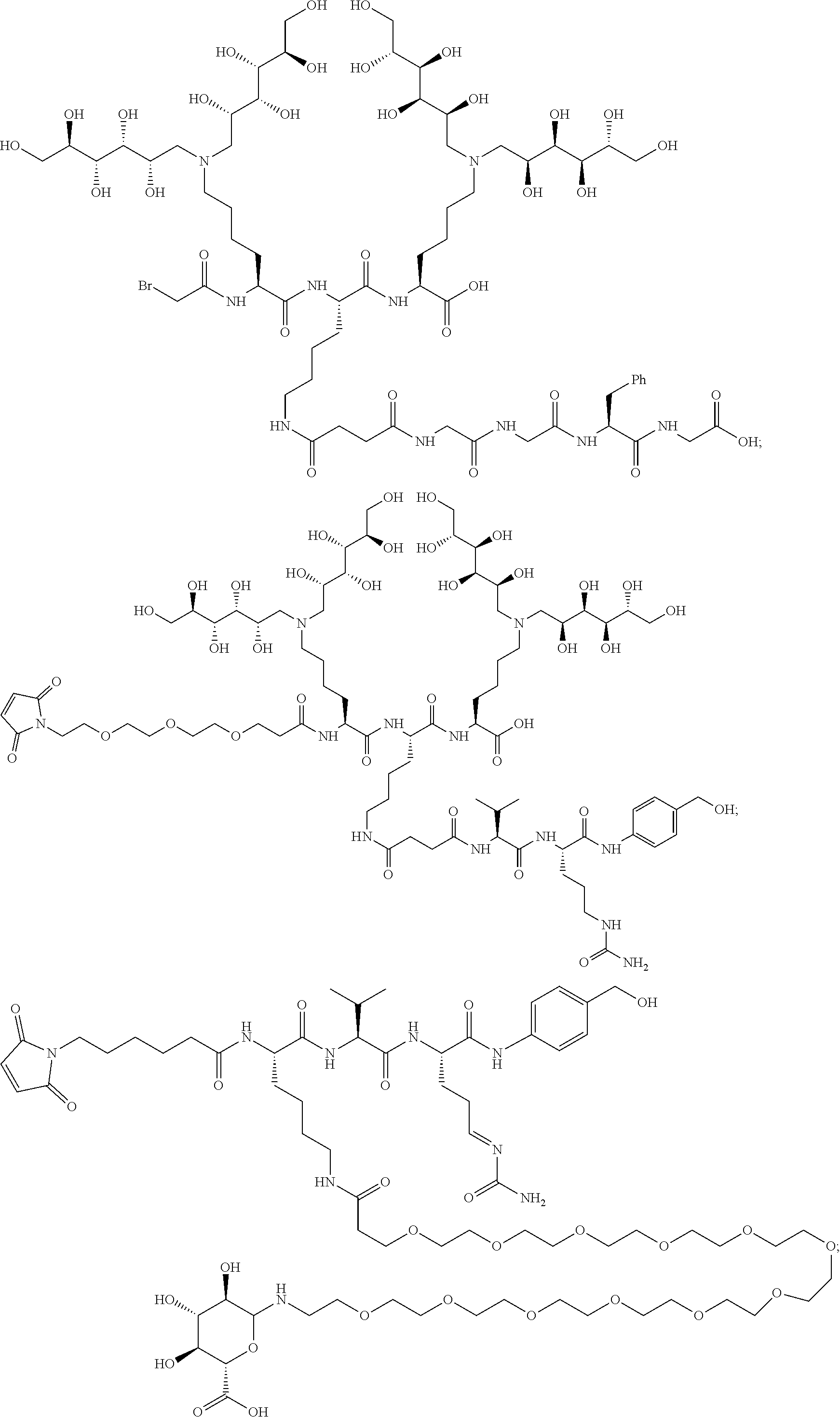

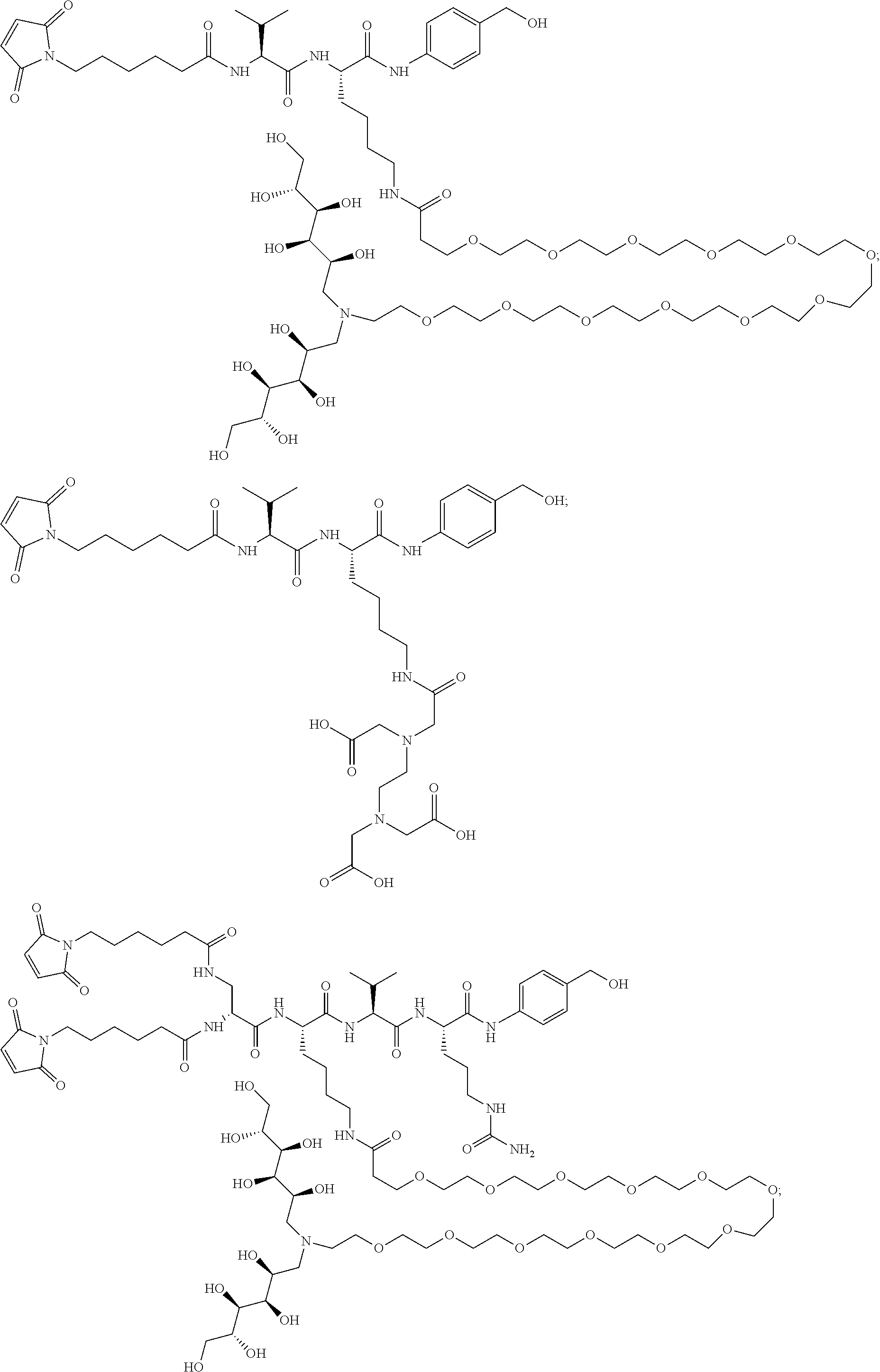

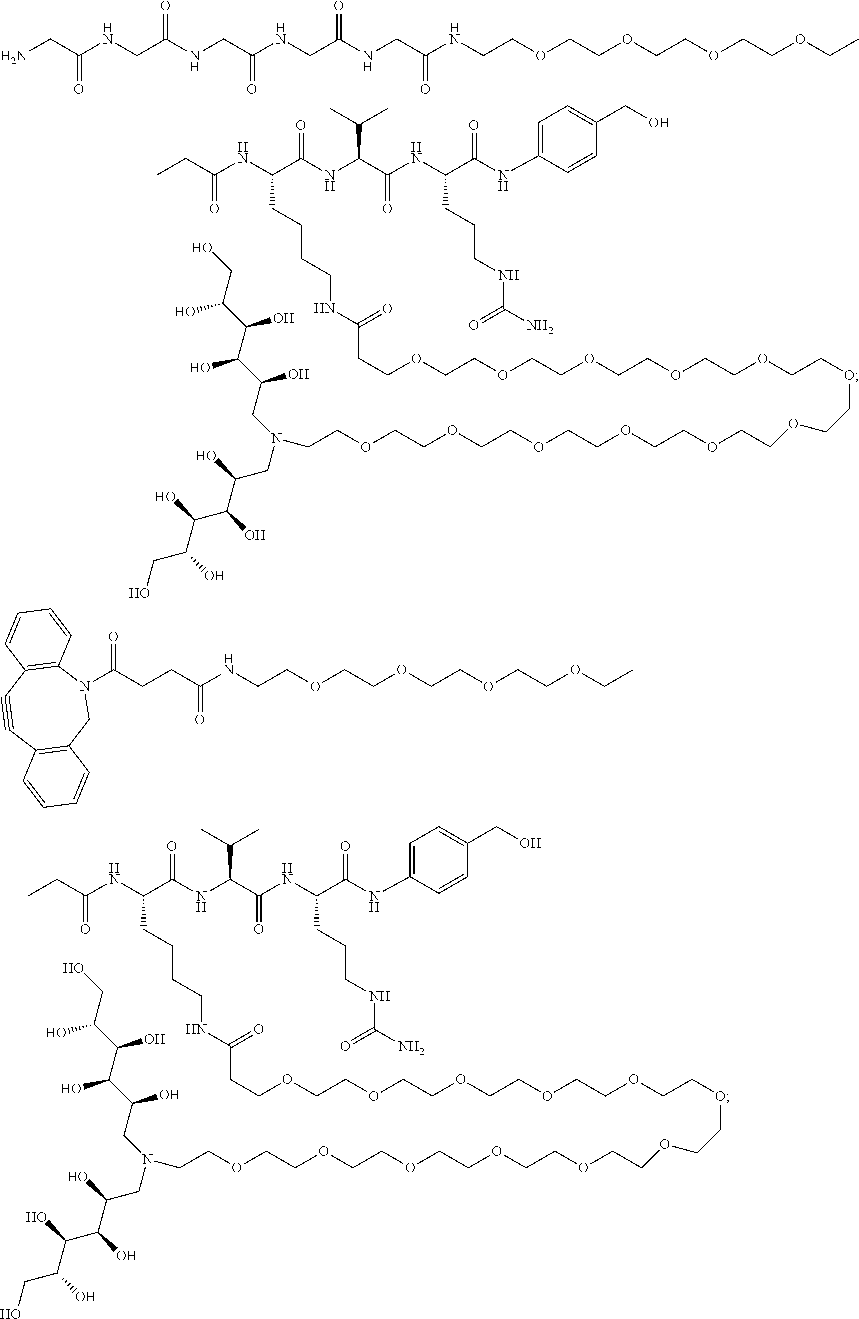

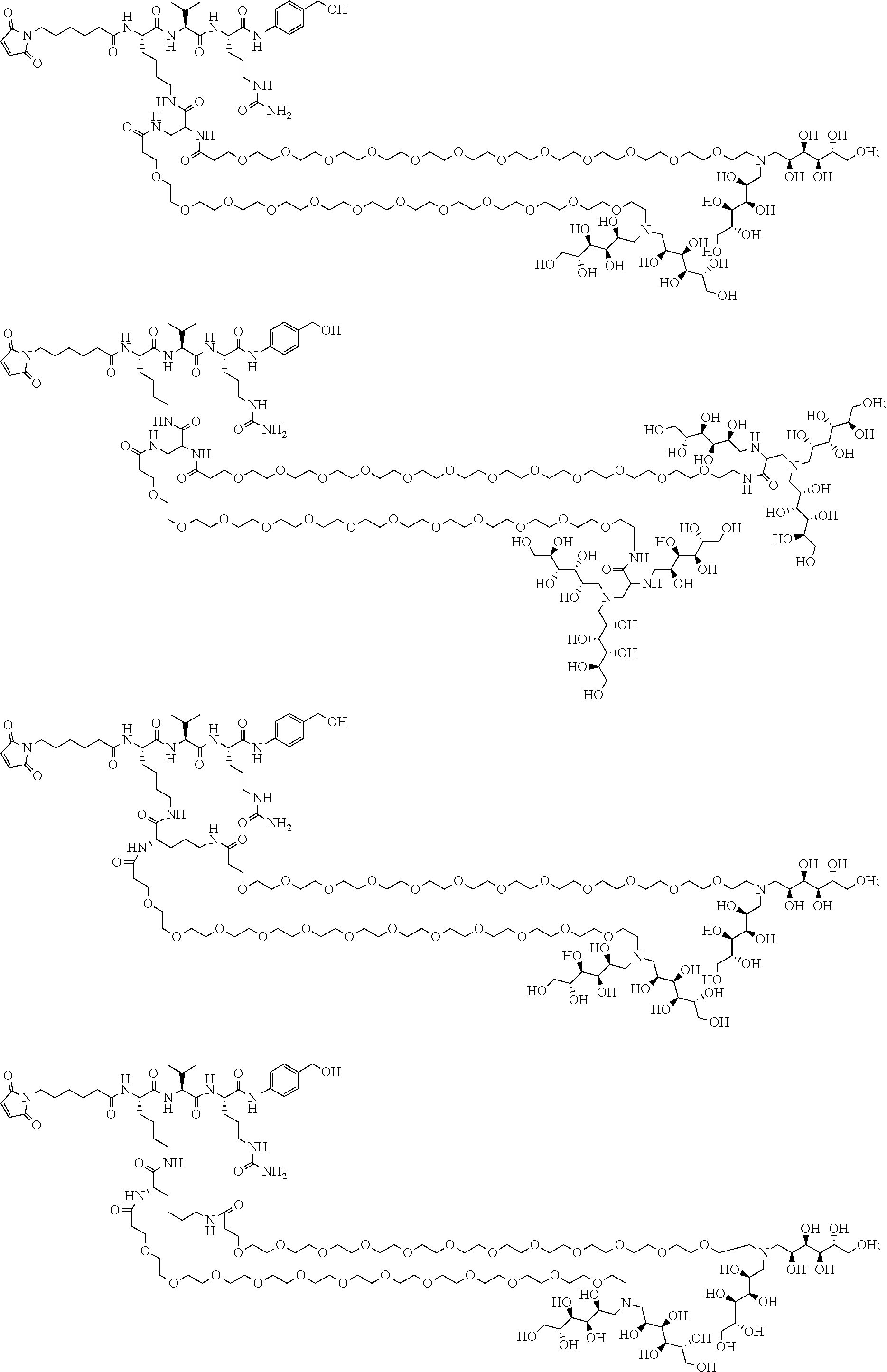

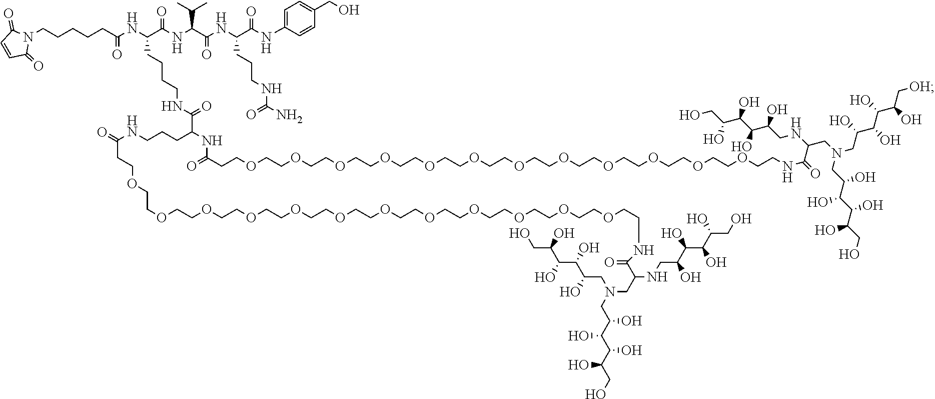

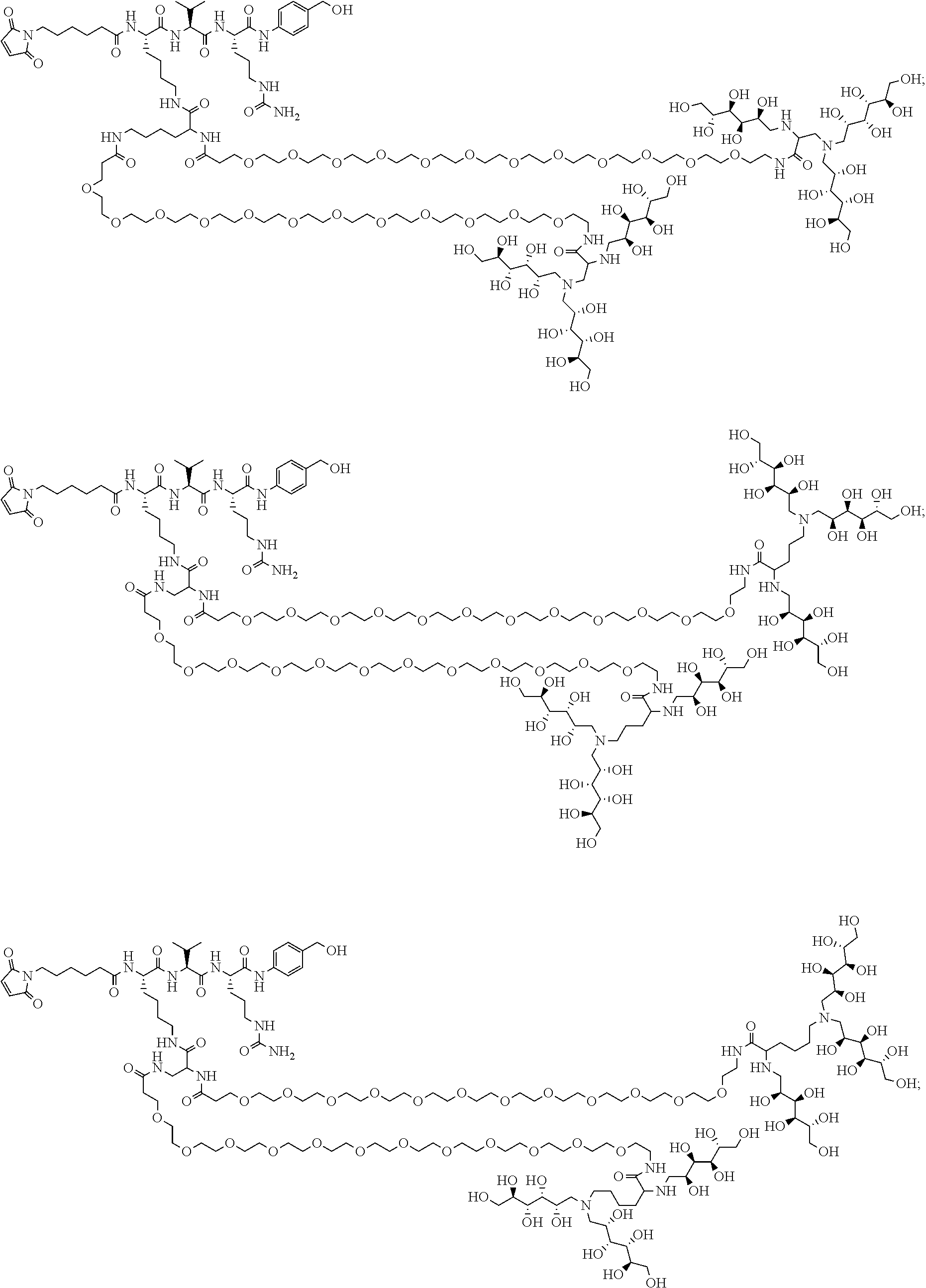

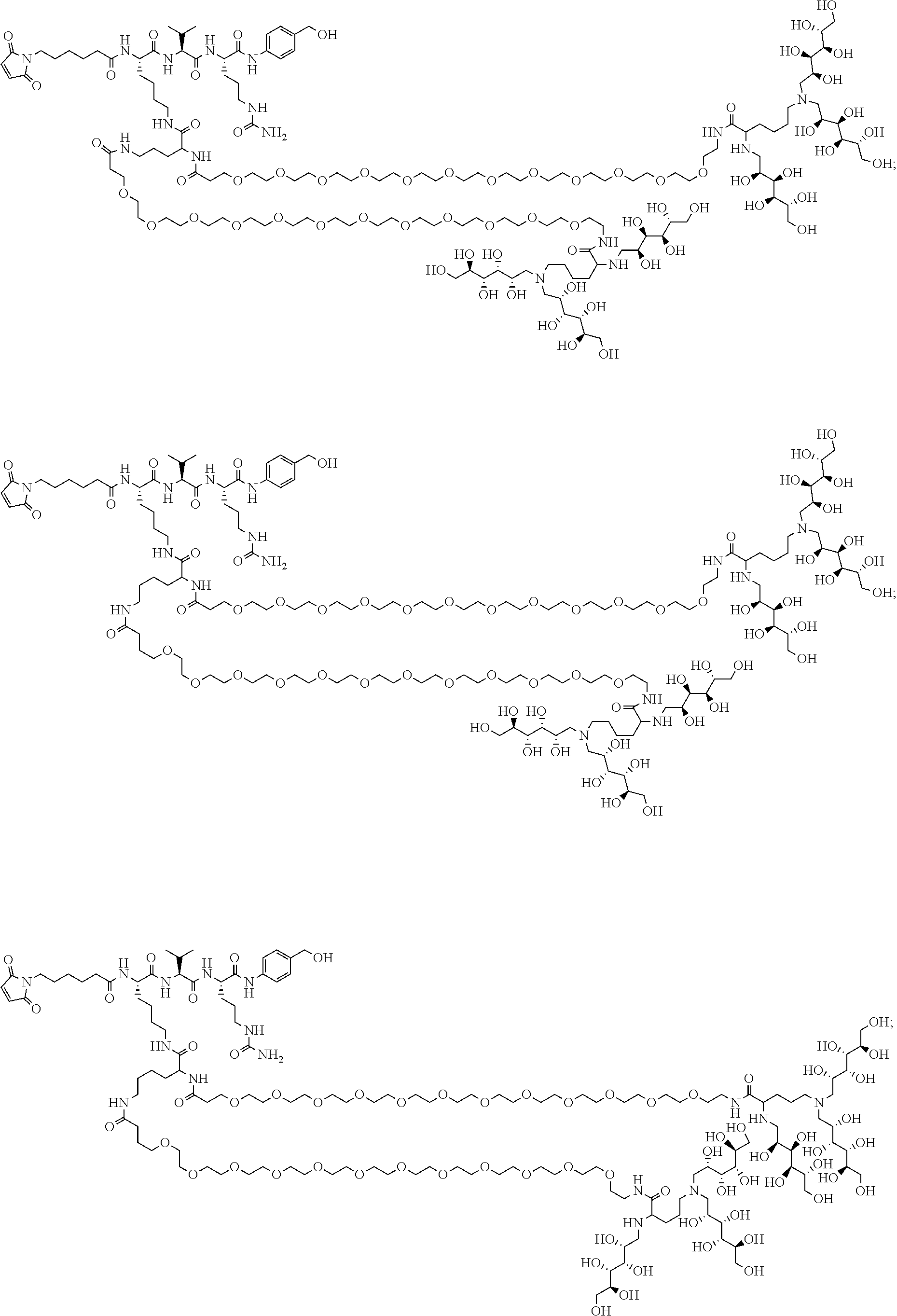

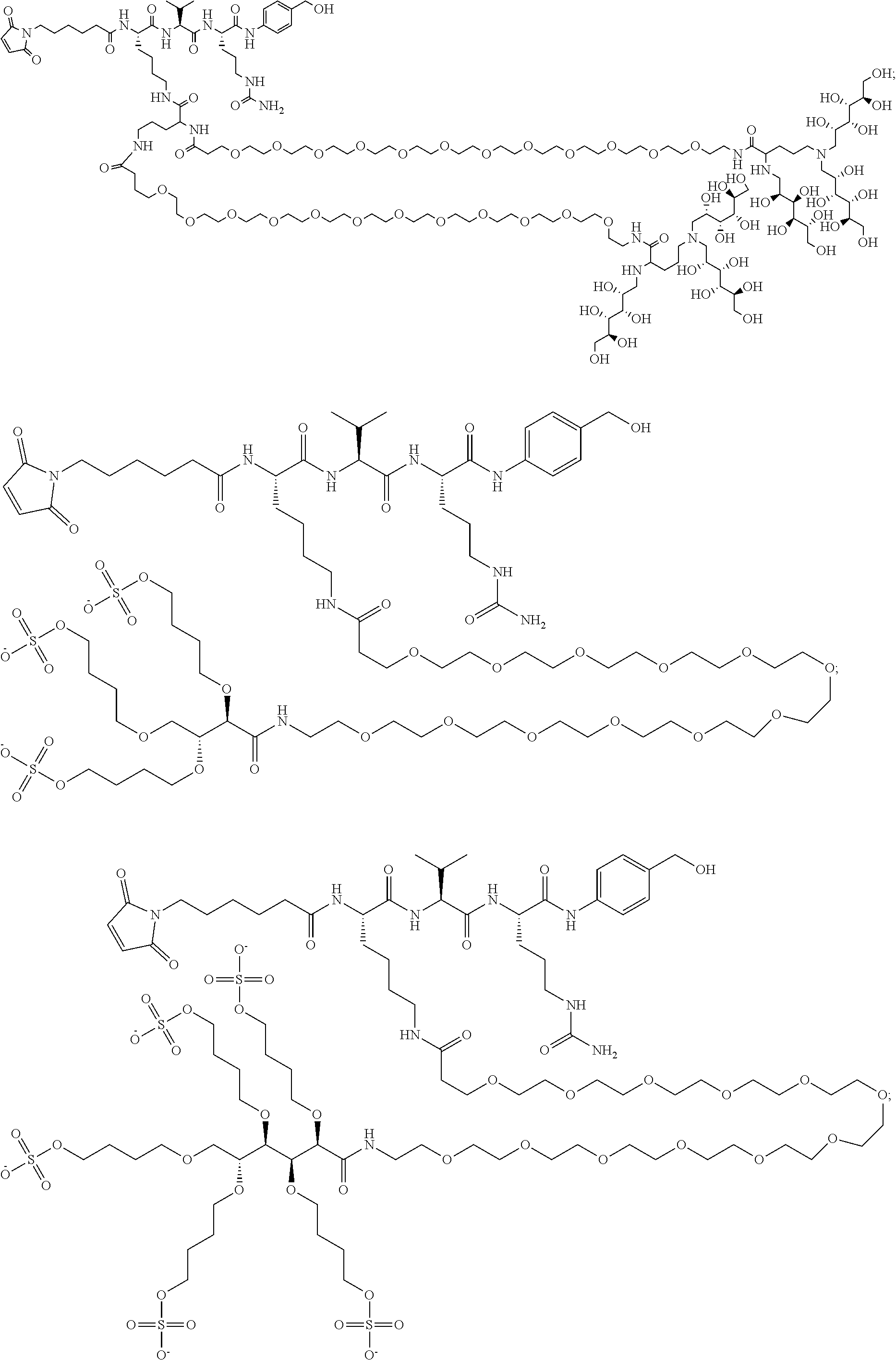

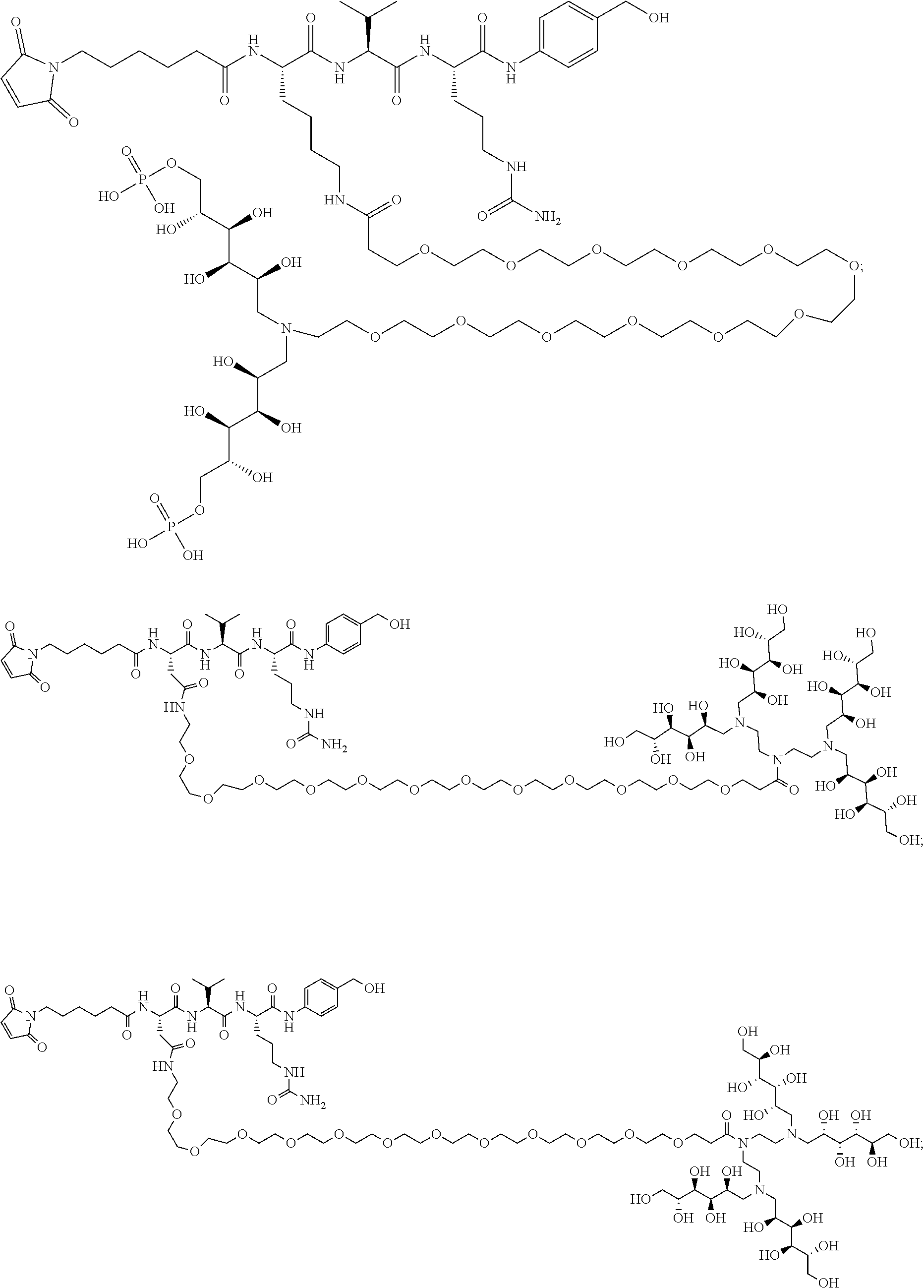

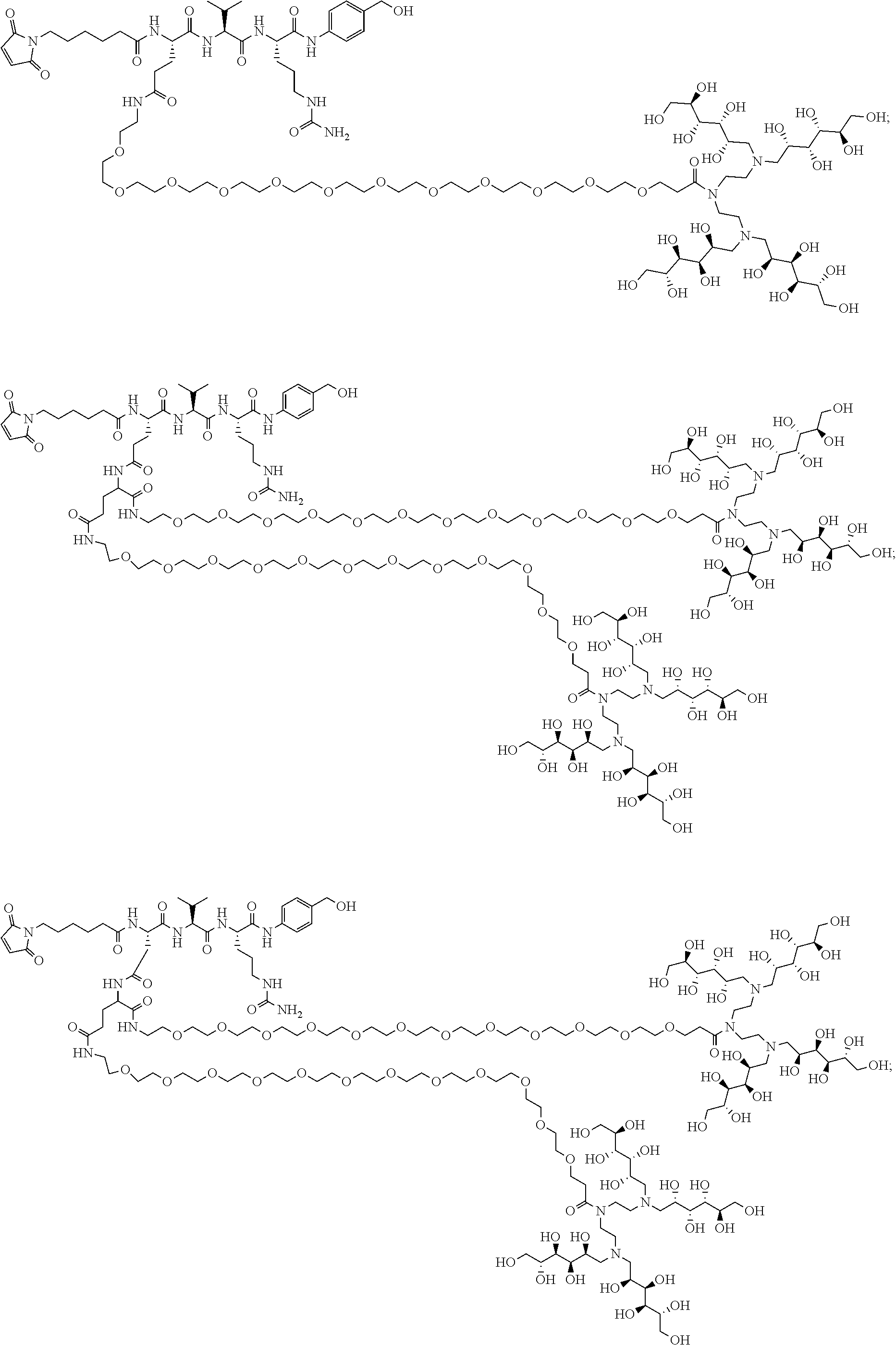

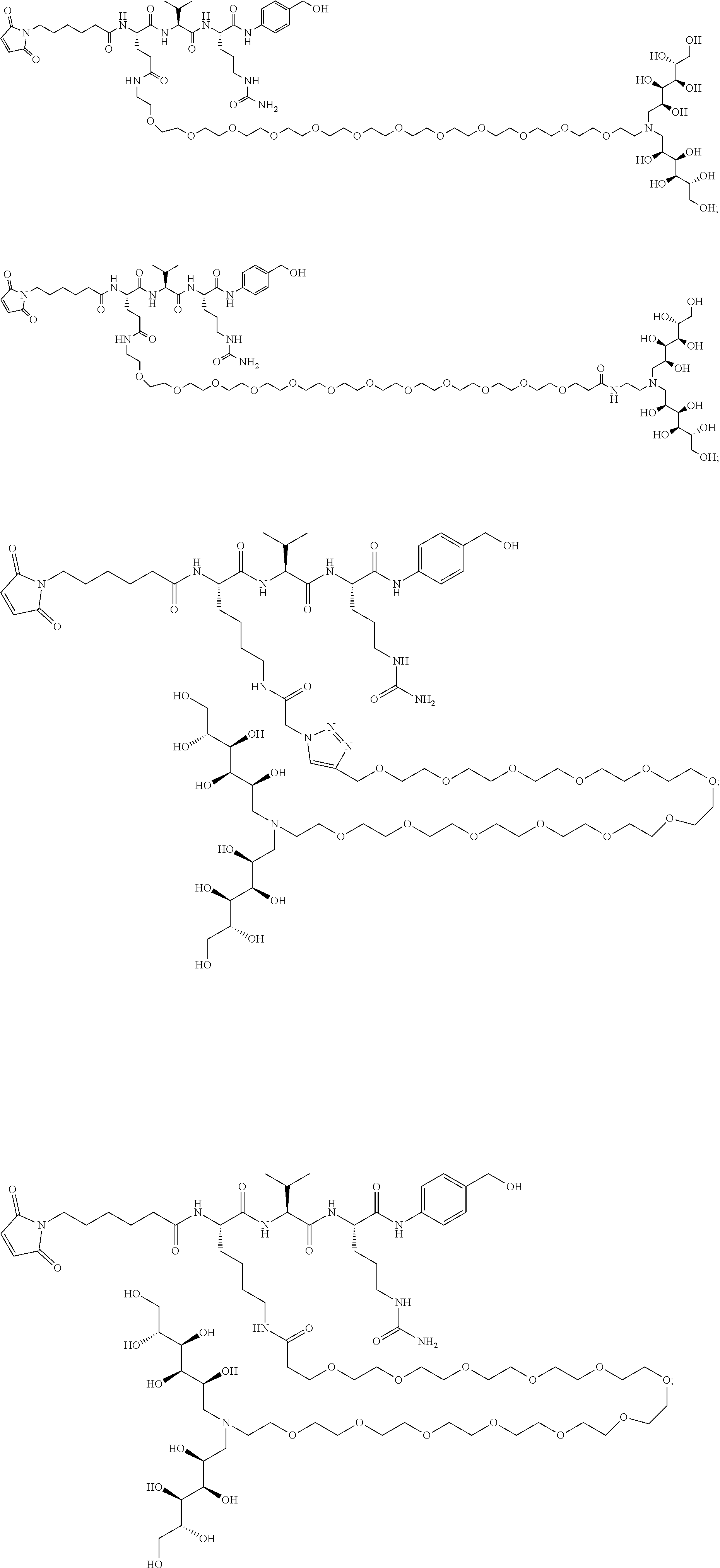

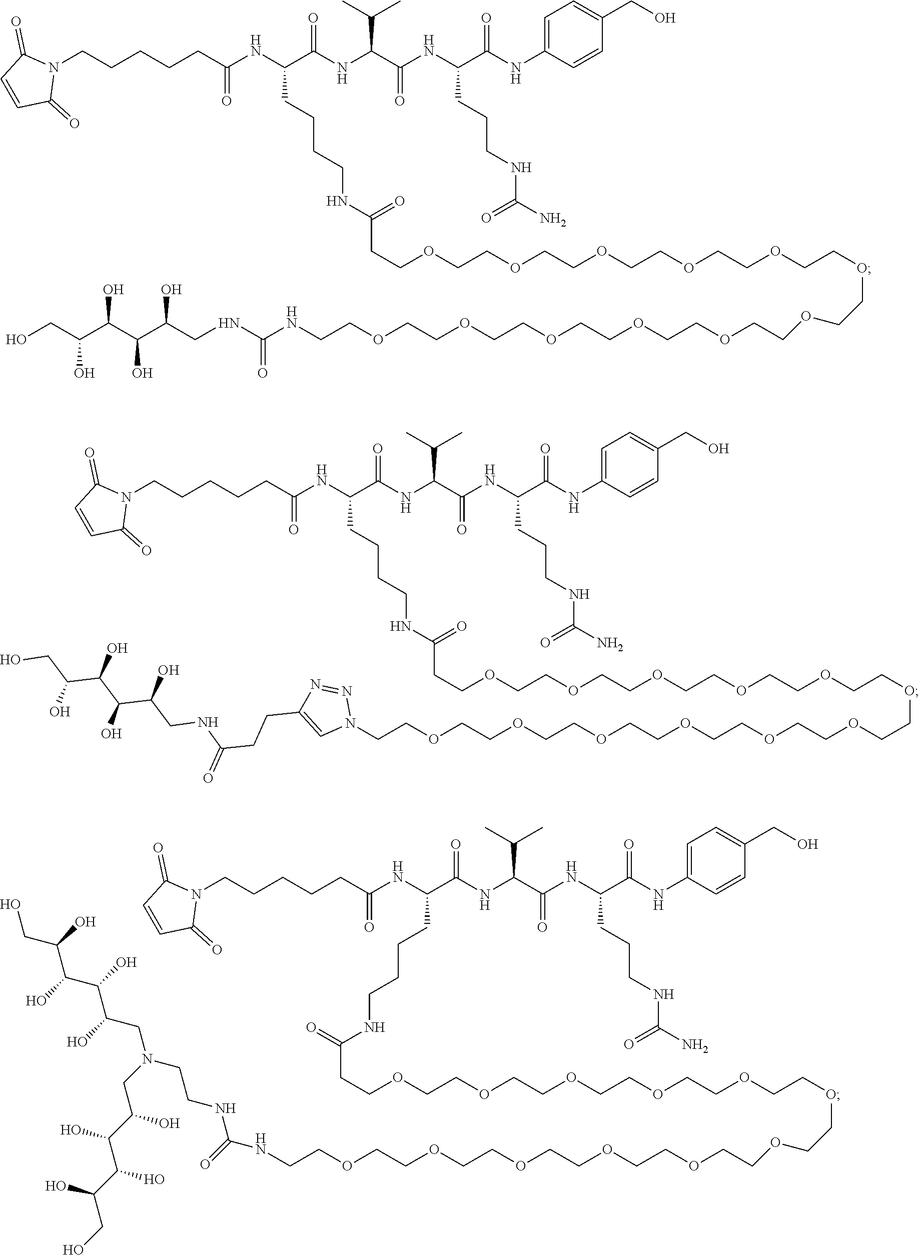

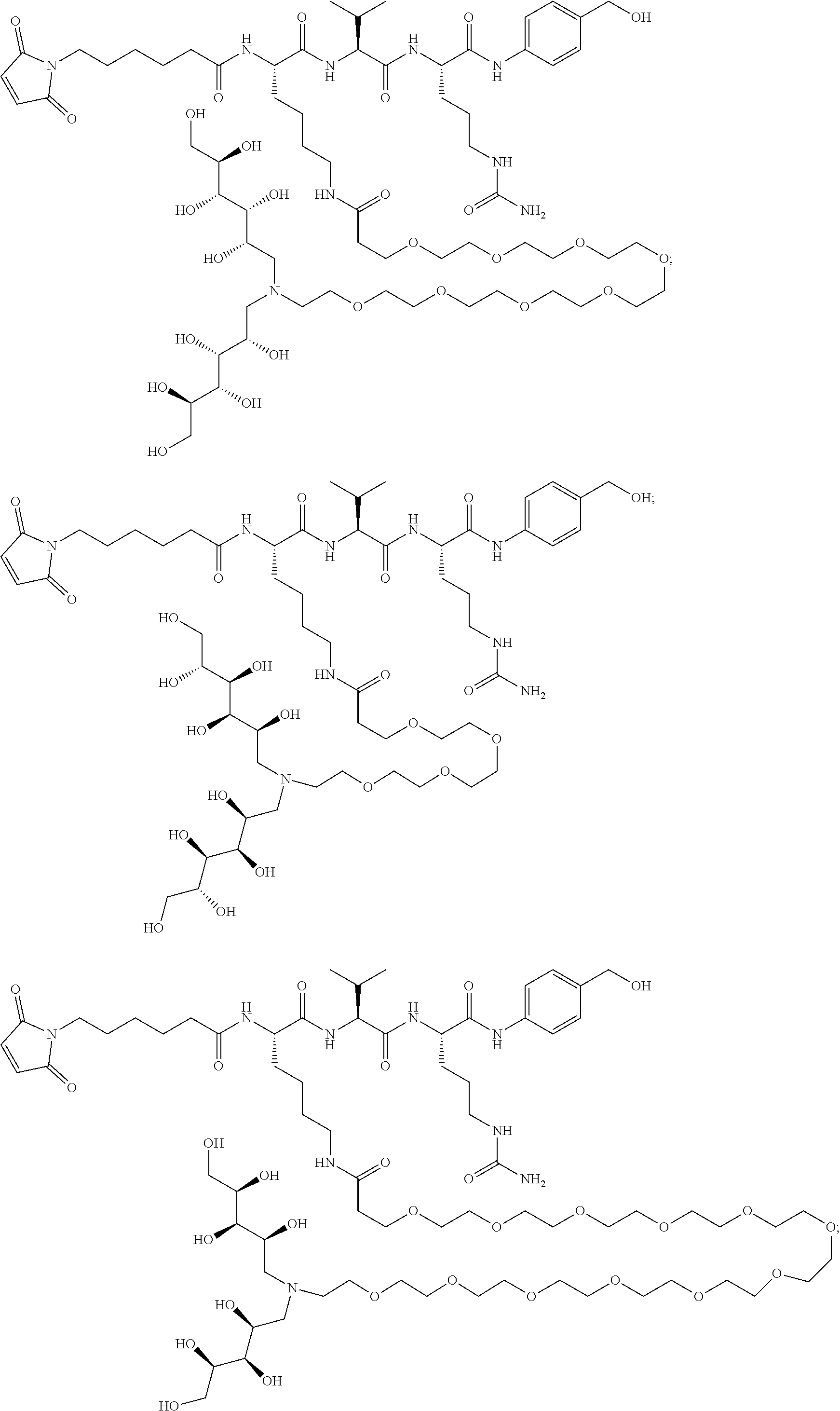

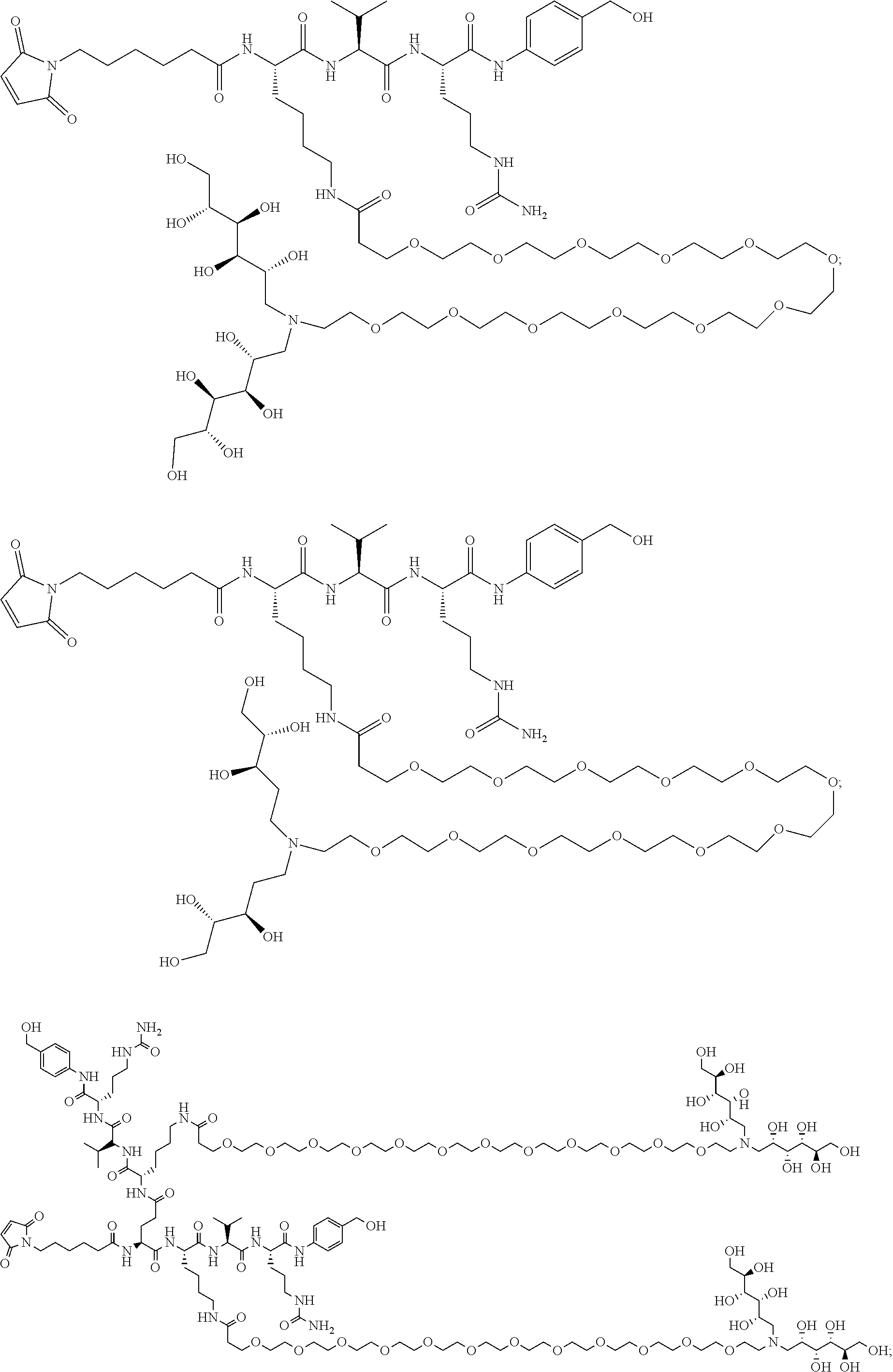

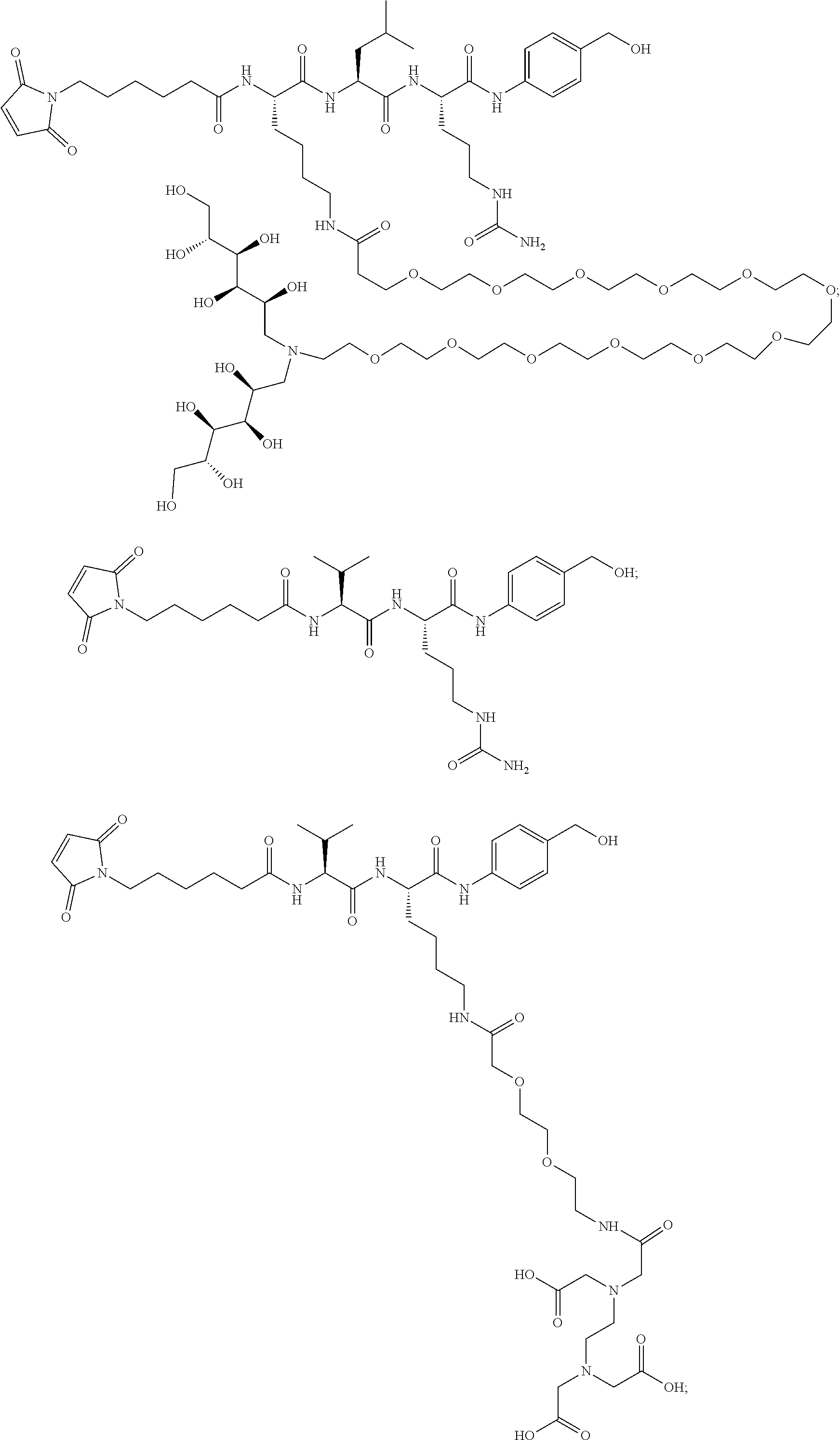

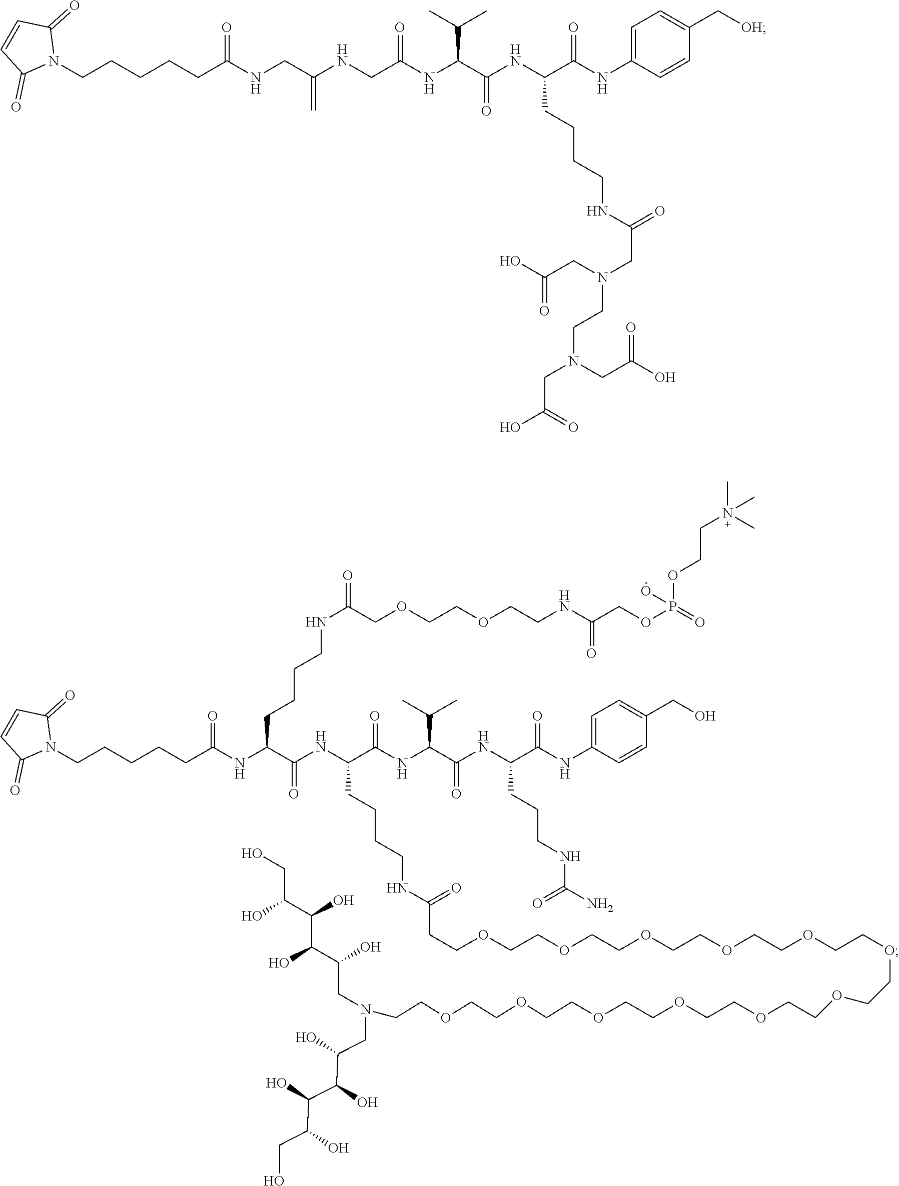

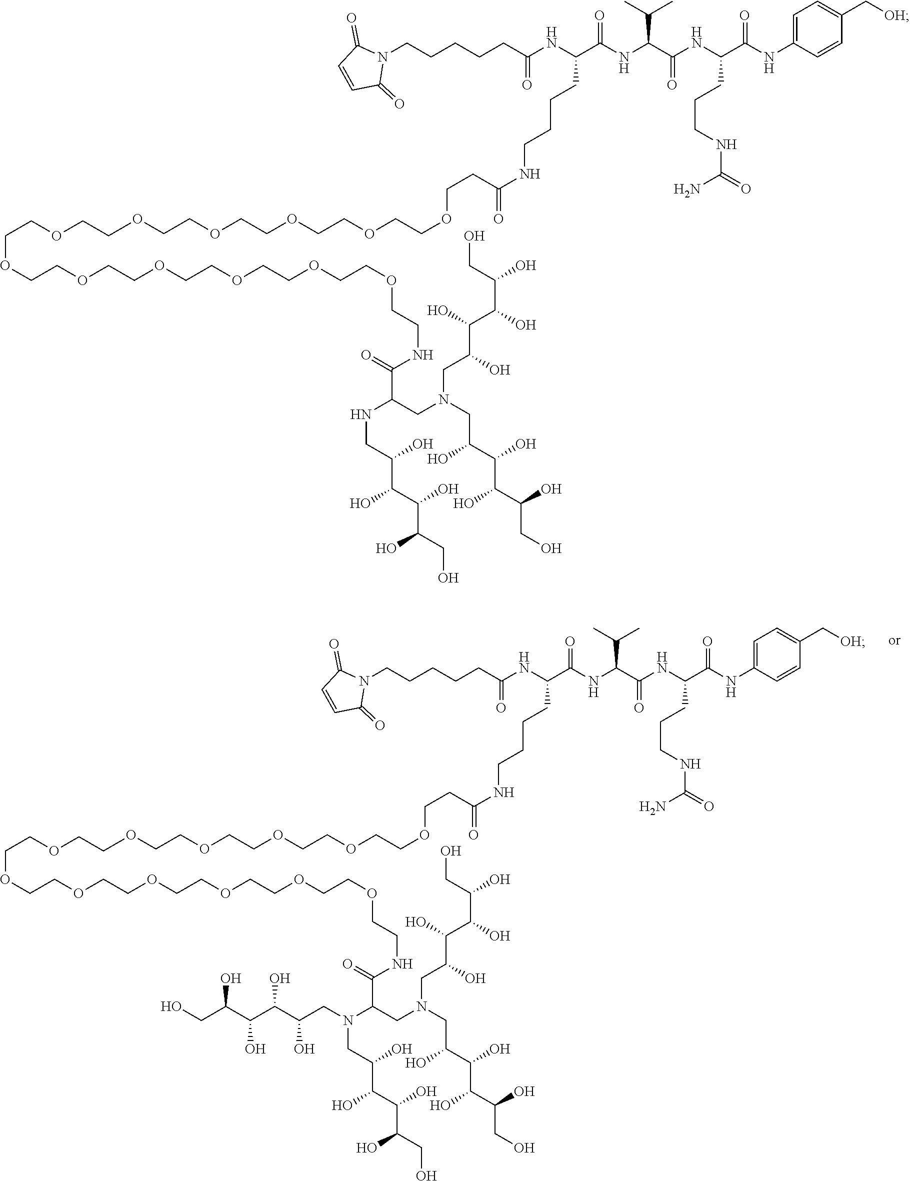

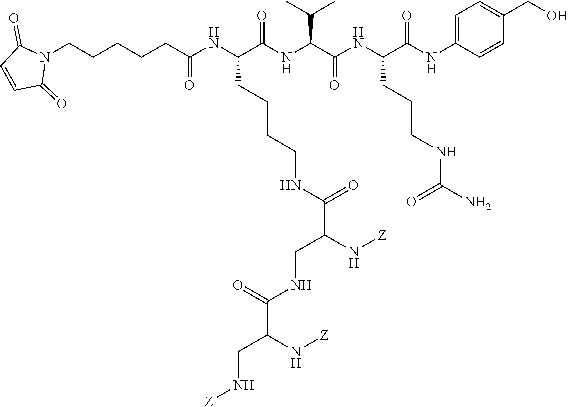

wherein each Z is attached at * and is individually selected from:

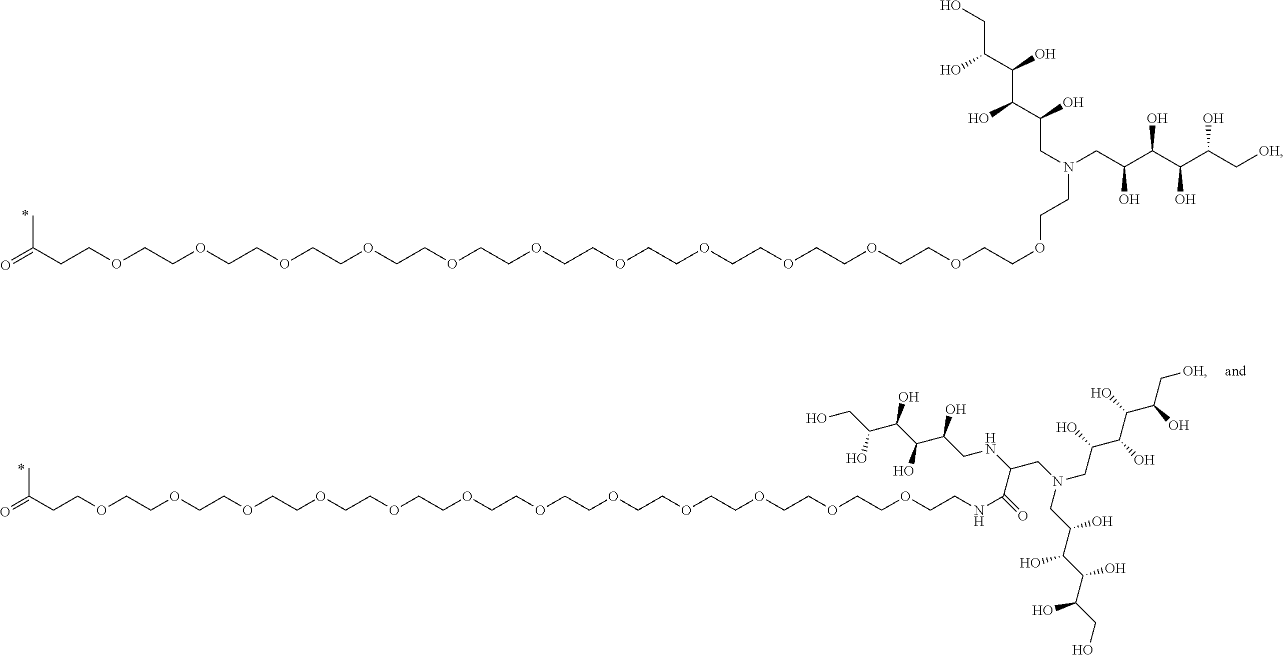

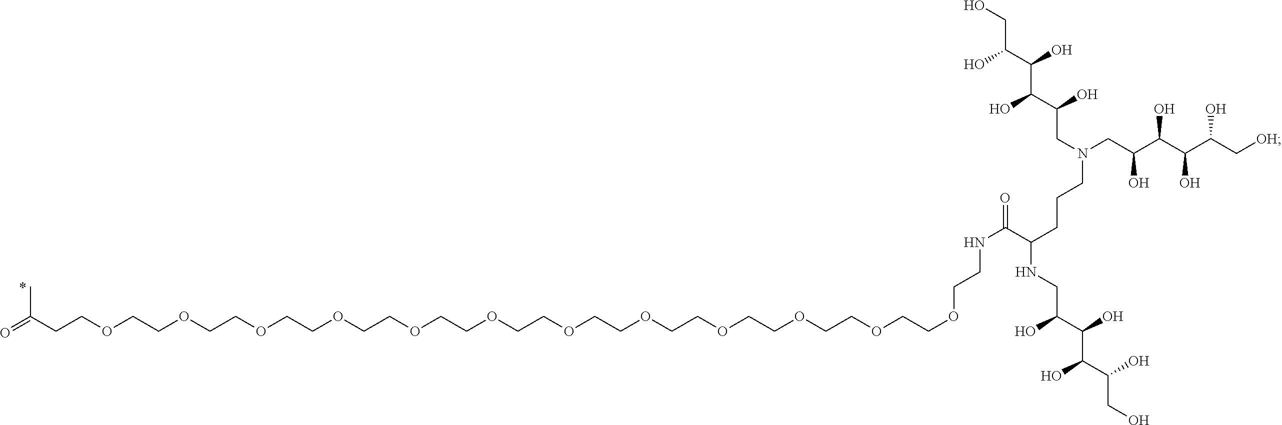

wherein a Drug unit is attached to the Linker Subunit L2, the terminal acid group or the benzyl alcohol, or wherein the wavy (≈) line indicates an attachment site for the Drug Unit.

In some embodiments, provided is a conjugate having the following

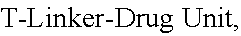

or a pharmaceutically acceptable salt thereof;
   wherein T is a Binding Unit;
   wherein the Linker has the following formula (I):

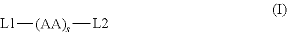

wherein:
(i) L1 is a Stretcher unit attached to the Binding unit,
(ii) AA is an Amino Acid unit having from 1 to 12 subunits;
(iii) s is 0 or 1;
(iv) L2 is a Linker Subunit attached to the Drug unit, wherein the Linker Subunit is a cleavable linker unit that comprises a cleavable peptide;
(v) Drug unit is selected from a cytotoxic agent, an immune modulatory agent, a nucleic acid, a growth inhibitory agent, a PROTAC, a toxin, a radioactive isotope and a chelating ligand; and
(vi) at least one PEG unit,
wherein the at least one PEG unit is present within the Amino Acid unit, the Linker Subunit, or combinations thereof, and wherein the at least one PEG unit has the formula:

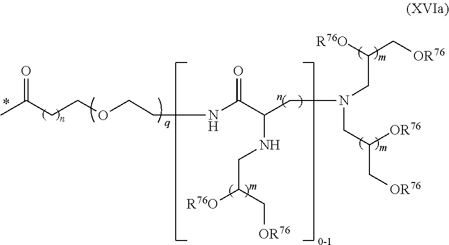

each R⁷⁶ is independently H, acetyl, —P(═O)(OH)₂, or —(CH₂)ᵥS(═O)₂(OH);
each q is independently 1-26;
each m is independently 1 to 4;
each n is independently 1 to 4;
each v is independently 1 to 6; and
each * indicates an attachment site for a subunit of the Linker Subunit, Amino Acid unit, or both

In some embodiments, provided is a Drug-Linker having the following formula (III)

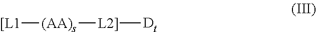

or a salt thereof, wherein:
   (i) L1 is a Stretcher unit;
   (ii) AA is an Amino Acid unit having from 1 to 12 subunits;
   (iii) s is 0 or 1;
   (iv) L2 is a Linker Subunit attached to the Drug unit (D), wherein the Linker Subunit is a cleavable linker unit that comprises a cleavable peptide, and wherein t is 1 to 4;
   (v) Drug unit is selected from a cytotoxic agent, an immune modulatory agent, a nucleic acid, a growth inhibitory agent, a PROTAC, a toxin, a radioactive isotope and a chelating ligand; and
   (vi) one or more PEG units,
wherein the one or more PEG units is present within the Amino Acid unit, the Linker Subunit, or combinations thereof, and wherein at least one of the one or more PEG units has the formula:

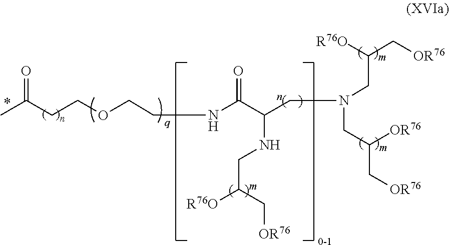

each R⁷⁶ is independently H, acetyl, —P(═O)(OH)₂, or —(CH₂)ᵥS(═O)₂(OH);
each q is independently 1-26;
each m is independently 1 to 4;
each n is independently 1 to 4;
each v is independently 1 to 6; and
each * indicates an attachment site to the Linker Subunit, Amino Acid unit, or both.

In some embodiments, for the conjugate, the Binding unit is an antibody or an antigen-binding portion thereof.

In some embodiments, for the conjugate, (1) the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO:21, SEQ ID NO:22, SEQ ID NO:13, SEQ ID NO:24, SEQ ID NO:25 and SEQ ID NO:26, respectively; SEQ ID NO:21, SEQ ID NO:22, SEQ ID NO:14, SEQ ID NO:24, SEQ ID NO:25 and SEQ ID NO:26, respectively; SEQ ID NO:21, SEQ ID NO:22, SEQ ID NO:15, SEQ ID NO:24, SEQ ID NO:25 and SEQ ID NO:26, respectively; SEQ ID NO:21, SEQ ID NO:22, SEQ ID NO:23, SEQ ID NO:24, SEQ ID NO:25 and SEQ ID NO:18, respectively; and SEQ ID NO:16, SEQ ID NO:17, SEQ ID NO:23, SEQ ID NO:24, SEQ ID NO:25 and SEQ ID NO:26.

In some embodiments, for the conjugate, the Binding unit is as described elsewhere herein.

In some embodiments, for the conjugate or Drug-Linker, each of the one or more PEG units has the formula:

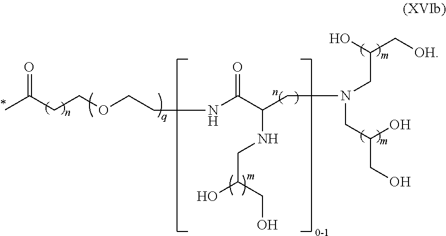

In some embodiments, for the conjugate or Drug-Linker, each of the one or more PEG unit has the formula

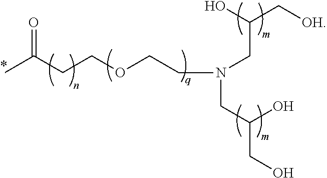

In some embodiments, for the conjugate or Drug-Linker, (vi) has one PEG unit. In some cases, (vi) has two PEG units.

In some embodiments, for the conjugate or Drug-Linker, q is independently 4-16. In some cases, q is 12. In some cases, m is 4. In some cases, n is 1.

In some embodiments, for the Drug-Linker, the Stretcher unit is capable of forming a bond with a sulfur atom. In some cases, the Stretcher unit comprises maleimido(C₁-C₁₀alkylene)-C(O)—, maleimido(CH₂OCH₂)ₚ₂(C₁-C₁₀alkylene)C(O)—, maleimido(C₁-C₁₀alkylene)(CH₂OCH₂)ₚ₂C(O)—, or a ring open form thereof, wherein p2 is from 1 to 26. In some cases, the Stretcher unit comprises maleimido(C₁-C₁₀alkylene)-C(O)—. In some cases, the Stretcher unit is maleimido(C₁-C₁₀alkylene)-C(O)—. In some cases, the Stretcher unit is

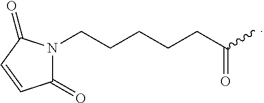

In some embodiments, for the conjugate or Drug-Linker, s is 0.

In some embodiments, for the conjugate or Drug-Linker, the cleavable peptide comprises a valine-citrulline peptide, a valine-alanine peptide, a valine-lysine peptide, a phenylalanine-lysine peptide, or a glycine-glycine-phenylalanine-glycine peptide. In some cases, the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG) peptide or a Lys(PEG)-glycine-glycine-phenylalanine-glycine peptide, wherein Lys(PEG) and Cit(PEG) comprise a PEG unit attached to a lysine residue or a citrulline residue, respectively, wherein the PEG unit is represented by the Formula (XVIb).

In some embodiments, for the conjugate or Drug-Linker, the cleavable peptide comprises a self immolative group. In some cases, the cleavable peptide comprises a para-aminobenzyl alcohol self immolative group (PABA) or a p-amino-benzyloxycarbonyl self immolative group, in some cases, the cleavable peptide comprises a p-amino-benzyloxycarbonyl self immolative group.

In some embodiments, for the conjugate or Drug-Linker, the cleavable peptide is attached to the Drug unit via the p-amino-benzyloxycarbonyl self immolative group.

In some embodiments, for the conjugate or Drug-Linker, s is 1.

In some embodiments, for the conjugate or Drug-Linker, the subunits of the Amino Acid unit are selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine. β-alanine, aminoalkanoic acid, aminoalkanoic acid, amino alkanedioic acid, aminobenzoic acid, amino-heterocyclo-alkanoic acid, heterocyclo-carboxylic acid, citrulline, and diaminoalkanoic acid; wherein the one or more PEG units is attached to one of the subunits. In some cases, the subunits of the Amino Acid unit are selected from alanine, arginine, aspartic acid, asparagine, histidine, glycine, glutamine, phenylalanine, lysine, leucine, serine, and citrulline. In some cases, the subunits of the Amino Acid unit are selected from lysine, valine, and citrulline. In some cases, each of the one or more PEG units has the formula selected from:

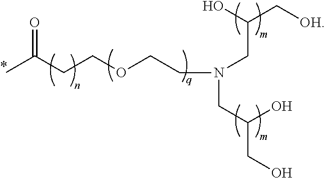

In some embodiments, for the conjugate or Drug-Linker, L2 is a cleavable peptide. In some cases, L2 is a cleavable peptide substituted with one or more PEG units.

In some embodiments, for the Drug-Linker, the Stretcher unit is selected from

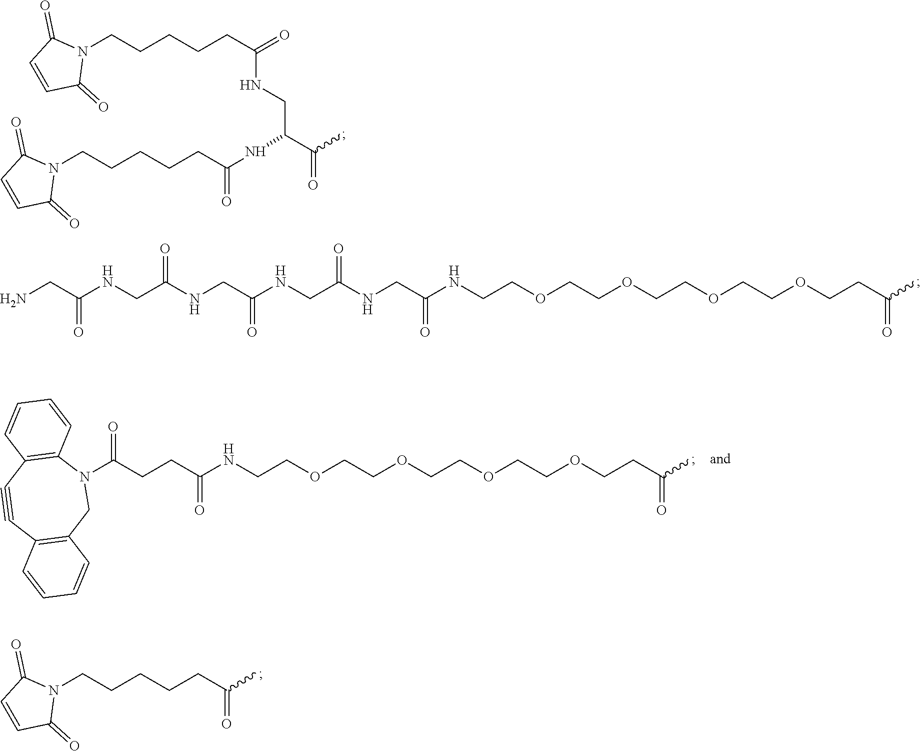

wherein the wavy line [symbol] indicates an attachment site of the Stretcher unit to the Amino Acid unit if s is 1 or L2 if s is 0.

In some embodiments, for the conjugate or Drug-Linker, the Drug unit is selected from a cytotoxic agent. In some cases, the cytotoxic agent is MMAE, MMAF, exatecan or SN-38, In some cases, the cytotoxic agent is exatecan. In some cases, the cytotoxic agent is MMAE. In some cases, the cytotoxic agent is MMAF.

In some embodiments, provided is a conjugate having the following formula (III*C)

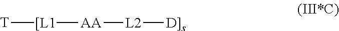

or a salt thereof, wherein:
   (vii) T is a Binding unit;
   (viii) s is p^{load}, wherein the p^{load} is selected from about 1 to about 16;
   (i) L1 is

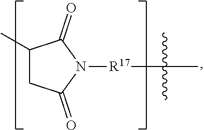

wherein R¹⁷ is C₁-C₈ alkylene-C(O)—;
(ii) AA is an Amino Acid unit having from 1 to 5 subunits;
(iii) wherein the 1 to 5 subunits of the Amino Acid unit are selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, and diaminoalkanoic acid;
(iv) L2 is a cleavable peptide covalently attached to a self immolative group;
(v) D is selected from a cytotoxic agent, an immune modulatory agent, a nucleic acid, a growth inhibitory agent, a PROTAC, a toxin, a radioactive isotope and a chelating ligand, wherein D is covalently attached to the self immolative group of L2; and
(vi) wherein one of the 1 to 5 subunits of the Amino Acid unit is covalently attached to a PEG unit, wherein the PEG unit has the formula:

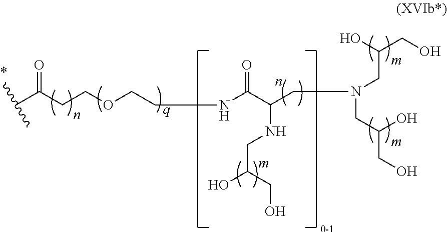

wherein each q is independently 1-26;
wherein each m is independently 1 to 4;
wherein each n is independently 1 to 4; and
wherein * indicates an attachment site to one of the 1 to 5 subunits of the Amino Acid unit.

In some embodiments, for the conjugate, the Binding unit is an antibody or an antigen-binding portion thereof.

In some embodiments, for the conjugate, (1) the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO:21, SEQ ID NO:22, SEQ ID NO:13, SEQ ID NO:24, SEQ ID NO:25 and SEQ ID NO:26, respectively; SEQ ID NO:21, SEQ ID NO:22, SEQ ID NO:14, SEQ ID NO:24, SEQ ID NO:25 and SEQ ID NO:26, respectively; SEQ ID NO:21, SEQ ID NO:22, SEQ ID NO:15, SEQ ID NO:24, SEQ ID NO:25 and SEQ ID NO:26, respectively; SEQ ID NO:21, SEQ ID NO:22, SEQ ID NO:23, SEQ ID NO:24, SEQ ID NO:25 and SEQ ID NO:18, respectively; and SEQ ID NO:16, SEQ ID NO:17, SEQ ID NO:23, SEQ ID NO:24, SEQ ID NO:25 and SEQ ID NO:26.

In some embodiments, the Binding unit may be as described elsewhere herein.

In some embodiments, for the conjugate, p^{load} is selected from about 8 to about 16. In some embodiments, p^{load} is about 8. In some embodiments, p^{load} is about 12. In some embodiments, p^{load} is about 16.

In some embodiments, provided is a Drug-Linker having the following formula (III*)

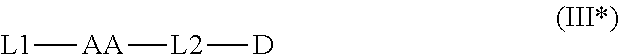

or a salt thereof, wherein:
   (i) L1 is maleimido (C₁-C₁₀alkylene)-C(O)—;
   (ii) AA is an Amino Acid unit having from 1 to 5 subunits;
   (iii) wherein the 1 to 5 subunits of the Amino Acid unit are selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine. β-alanine, aminoalkanoic acid, aminoalkynoic acid, amino alkanedioic acid, aminobenzoic acid, amino-heterocyclo-alkanoic acid, heterocyclo-carboxylic acid, citrulline, and diaminoalkanoic acid;
   (iv) L2 is a cleavable peptide covalently attached to a self immolative group;
   (v) D is selected from a cytotoxic agent, an immune modulatory agent, a nucleic acid, a growth inhibitory agent, a PROTAC, a toxin, a radioactive isotope and a chelating ligand, wherein D is covalently attached to the self immolative group of L2; and
   (vi) wherein one of the 1 to 5 subunits of the Amino Acid unit is covalently attached to a PEG unit, wherein the PEG unit has the formula:

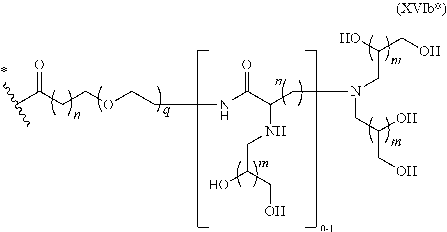

wherein each q is independently 1-26;
wherein each m is independently 1 to 4;
wherein each n is independently 1 to 4; and
wherein * indicates an attachment site to one of the 1 to 5 subunits of the Amino Acid unit.

In some embodiments, for the conjugate or Drug-Linker, D is a cytotoxic agent. In some cases, the cytotoxic agent is MMAE, MMAF, exatecan or SN-38. In some cases, the cytotoxic agent is exatecan. In some cases, D is

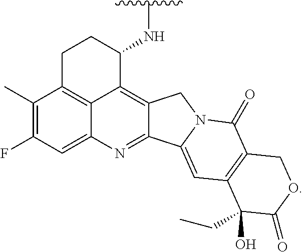 [SMILES: *N[C@H]1CCc2c(C)c(F)cc3nc4c(c1c23)Cn1c-4cc2c(c1=O)COC(=O)[C@]2(O)CC]

In some cases, D is

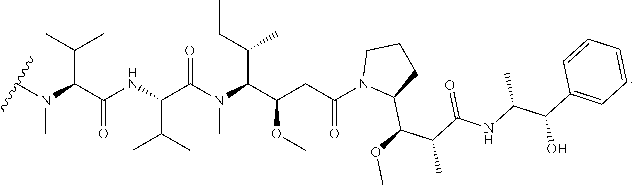 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, for the conjugate or Drug-Linker, the cleavable peptide is selected from a valine-citrulline peptide, a valine-alanine peptide, a valine-lysine peptide, a phenylalanine-lysine peptide, and a glycine-glycine-phenylalanine-glycine peptide. In some cases, the cleavable peptide is selected from

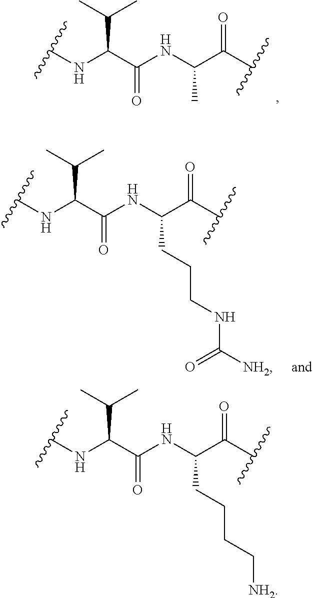

In some cases, the cleavable peptide is

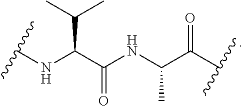 [SMILES: *N[C@H](C(=O)N[C@@H](C)C(*)=O)C(C)C]

In some cases, the cleavable peptide is

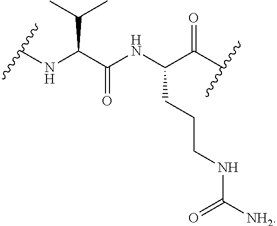 [SMILES: *N[C@H](C(=O)N[C@@H](CCCNC(N)=O)C(*)=O)C(C)C]

In some cases, the cleavable peptide is

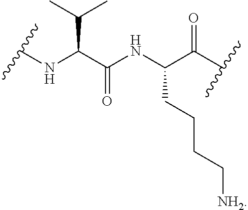 [SMILES: *N[C@H](C(=O)N[C@@H](CCCCN)C(*)=O)C(C)C]

In some embodiments, for the conjugate or Drug-Linker, the self immolative group is selected from para-aminobenzyl alcohol self immolative group (PABA) and p-amino-benzyloxycarbonyl self immolative group. In some cases, the self immolative group is selected from

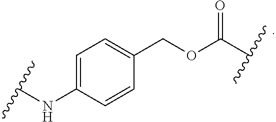 [SMILES: *Nc1ccc(COC(*)=O)cc1]

In some embodiments, L2 is selected from

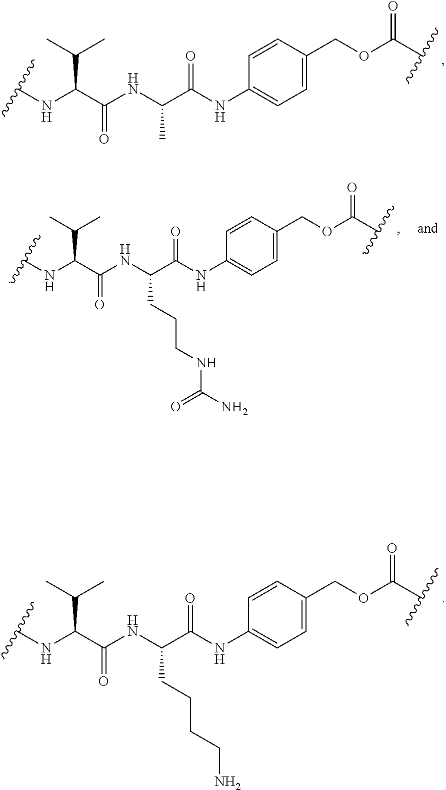 [SMILES: *N[C@H](C(=O)N[C@@H](C)C(=O)Nc1ccc(COC(*)=O)cc1)C(C)C ; *N[C@H](C(=O)N[C@@H](CCCNC(N)=O)C(=O)Nc1ccc(COC(*)=O)cc1)C(C)C ; *N[C@H](C(=O)N[C@@H](CCCCN)C(=O)Nc1ccc(COC(*)=O)cc1)C(C)C]

In some cases, L2 is

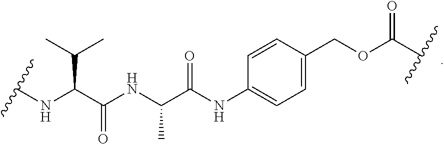 [SMILES: *N[C@H](C(=O)N[C@@H](C)C(=O)Nc1ccc(COC(*)=O)cc1)C(C)C]

In some cases, L2 is

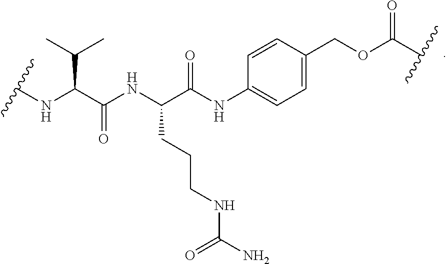 [SMILES: *N[C@H](C(=O)N[C@@H](CCCNC(N)=O)C(=O)Nc1ccc(COC(*)=O)cc1)C(C)C]

In some cases, L2 is

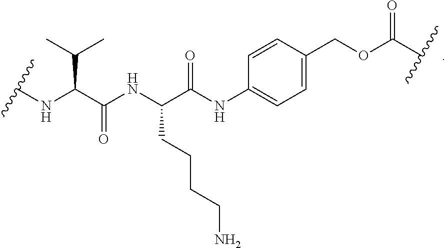 [SMILES: *N[C@H](C(=O)N[C@@H](CCCCN)C(=O)Nc1ccc(COC(*)=O)cc1)C(C)C]

In some embodiments, for the Drug-Linker, L1 is

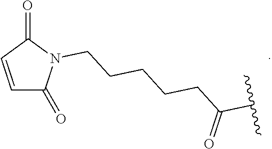 [SMILES: *C(=O)CCCCCN1C(=O)C=CC1=O ; "."]

In some embodiments, for the conjugate or Drug-Linker, the 1 to 5 subunits of the Amino Acid unit are selected from alanine, arginine, asparagine, histidine, glycine, glutamine, phenylalanine, lysine, leucine, serine, tyrosine, threonine, isoleucine, proline, tryptophan, valine, omithine, β-alanine, and citrulline. In some cases, the 1 to 5 subunits of the Amino Acid unit are selected from alanine, arginine, asparagine, histidine, glycine, glutamine, phenylalanine, lysine, leucine, serine, tyrosine, threonine, isoleucine, proline, tryptophan, valine, ornithine, β-alanine, and citrulline. In some cases, the Amino Acid unit has 1 subunit. In some cases, the Amino Acid unit has 2 subunits. In some cases, the Amino Acid unit has 3 subunits.

In some embodiments, for the conjugate or Drug-Linker, AA is selected from

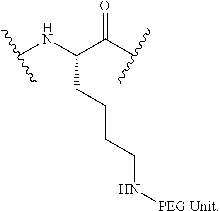

In some cases, AA is selected from

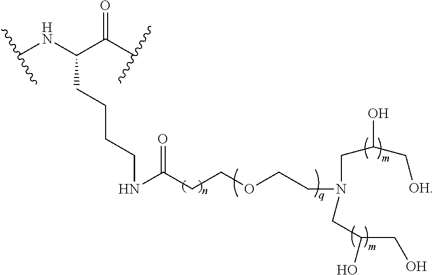

In some cases, q is independently 1-16. In some cases, each m is independently 3 to 4. In some cases, each n is independently 1 to 2. In some cases, n is 1. In some cases, each m is 4. In some cases, q is selected from 4, 8, and 12. In some cases, q is 12.

In some embodiments, provided is a conjugate, wherein the conjugate includes the Drug-Linker of formula (III*) and a Binding unit. In some cases, the Binding unit includes a reactive substituent which reacts with the maleimido of Drug-Linker of formula (III*) to form a new covalent bond and thus forming the conjugate.

In some embodiments, provided is a conjugate, wherein the conjugate includes the Drug-Linker of formula (III) and a Binding unit, wherein the Drug-Linker of formula (III). In some cases, the Binding unit includes a reactive substituent which reacts with the maleimido of Drug-Linker of formula (III) to form a new covalent bond and thus forming the conjugate.

In some embodiments, provided is a conjugate comprising a Linker, further comprising at least one Drug unit attached to Linker Subunit L2 to form a Drug-Linker. In some embodiments, provided is a conjugate comprising a Drug-Linker, wherein the Drug unit is selected from a cytotoxic agent, an immune modulatory agent, a nucleic acid, a growth inhibitory agent, a PROTAC, a toxin, a radioactive isotope and a chelating ligand. In some embodiments, provided is a conjugate comprising a Drug-Linker, wherein the Drug unit is a cytotoxic agent. In some embodiments, provided is a conjugate comprising a Drug-Linker, wherein the cytotoxic agent is selected from the group consisting of an auristatin, a maytansinoid, a camptothecin, a duocarmycin, and a calicheamicin. In some embodiments, provided is a conjugate comprising a Drug-Linker, wherein the cytotoxic agent is an auristatin. In some embodiments, provided is a conjugate comprising a Drug-Linker, wherein the cytotoxic agent is MMAE or MMAF. In some embodiments, provided is a conjugate comprising a Drug-Linker, wherein the cytotoxic agent is a camptothecin. In some embodiments, provided is a conjugate comprising a Drug-Linker, wherein the cytotoxic agent is exatecan or SN-38. In some embodiments, provided is a conjugate comprising a Drug-Linker, wherein the cytotoxic agent is exatecan (SS). In some embodiments, provided is a conjugate comprising a Drug-Linker, wherein the cytotoxic agent is diastereoisomer of exatecan, the RS form. In some embodiments, provided is a conjugate comprising a Drug-Linker, wherein the cytotoxic agent is a calicheamicin. In some embodiments, provided is a conjugate comprising a Drug-Linker, wherein the cytotoxic agent is a maytansinoid. In some embodiments, provided is a conjugate comprising a Drug-Linker, wherein the maytansinoid is maytansine, maytansinol or ansamatocin-2.

In some embodiments, provided is a conjugate comprising a Drug-Linker, wherein the Drug unit is an immune modulatory agent. In some embodiments, provided is a conjugate comprising a Drug-Linker, wherein the immune modulatory agent is selected from a TRL7 agonist, a TLR8 agonist, a STING agonist, or a RIG-I agonist. In some embodiments, provided is a conjugate comprising a Drug-Linker, wherein the immune modulatory agent is an TLR7 agonist. In some embodiments, provided is a conjugate comprising a Drug-Linker, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3. In some embodiments, provided is a conjugate comprising a Drug-Linker, wherein the immune modulatory agent is a TLR8 agonist. In some embodiments, provided is a conjugate comprising a Drug-Linker, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, provided is a Drug-Linker, wherein the immune modulatory agent is a STING agonist. In some embodiments, provided is a conjugate comprising a Drug-Linker, wherein the immune modulatory agent is a RIG-I agonist. In some embodiments, provided is a conjugate comprising a Drug-Linker, wherein the RIG-I agonist is selected from KIN1148, SB-9200, KIN700, KIN600, KIN500, KIN100, KIN101, KIN400 and KIN2000.

In some embodiments, provided is a Drug-Linker, wherein the Drug unit is a chelating ligand. In some embodiments, provided is a Drug-Linker, wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

In some embodiments, provided is a conjugate comprising a Binding unit attached to any of the Drug-Linkers described herein. In some embodiments, provided is a conjugate, wherein the Binding unit is selected from an antibody or an antigen-binding portion thereof. In some embodiments, provided is a conjugate, wherein the Binding unit is a monoclonal antibody, a Fab, a Fab′, an F(ab′), an Fv, a disulfide linked Fc, a scFv, a single domain antibody, a diabody, a bi-specific antibody, or a multi-specific antibody. In some embodiments, provided is a conjugate, wherein the Binding unit is mono-specific. In some embodiments, provided is a conjugate, wherein the Binding unit is bivalent. In some embodiments, provided is a conjugate, wherein the Binding unit is bispecific.

In some embodiments, provided is a conjugate, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

In some embodiments, provided is a conjugate, selected from the following:

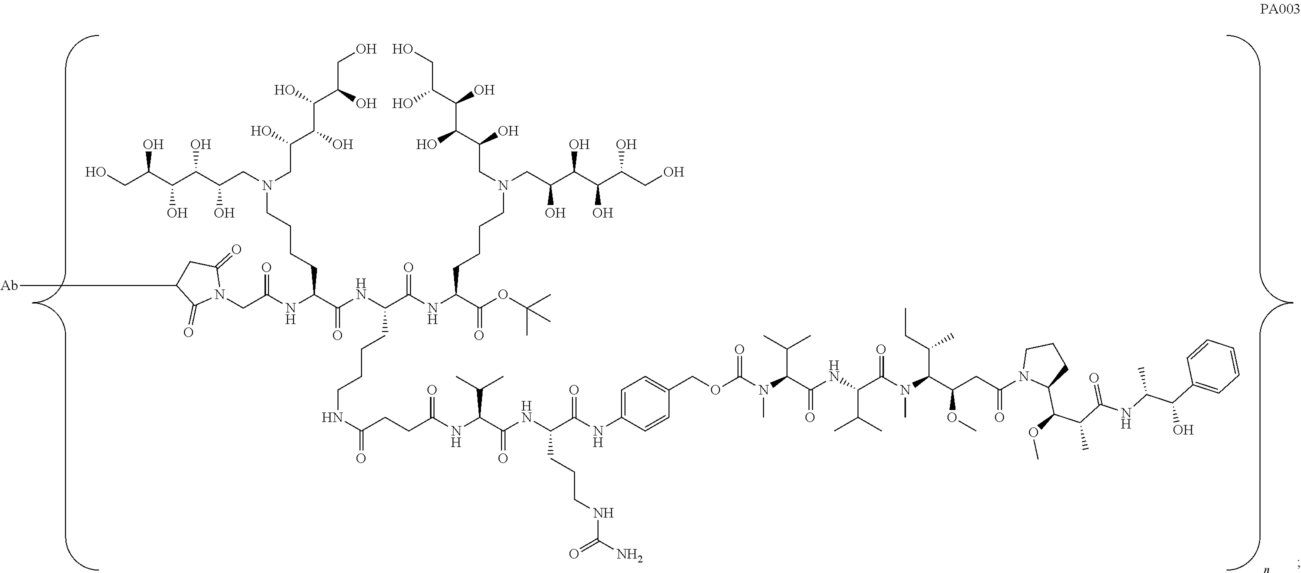

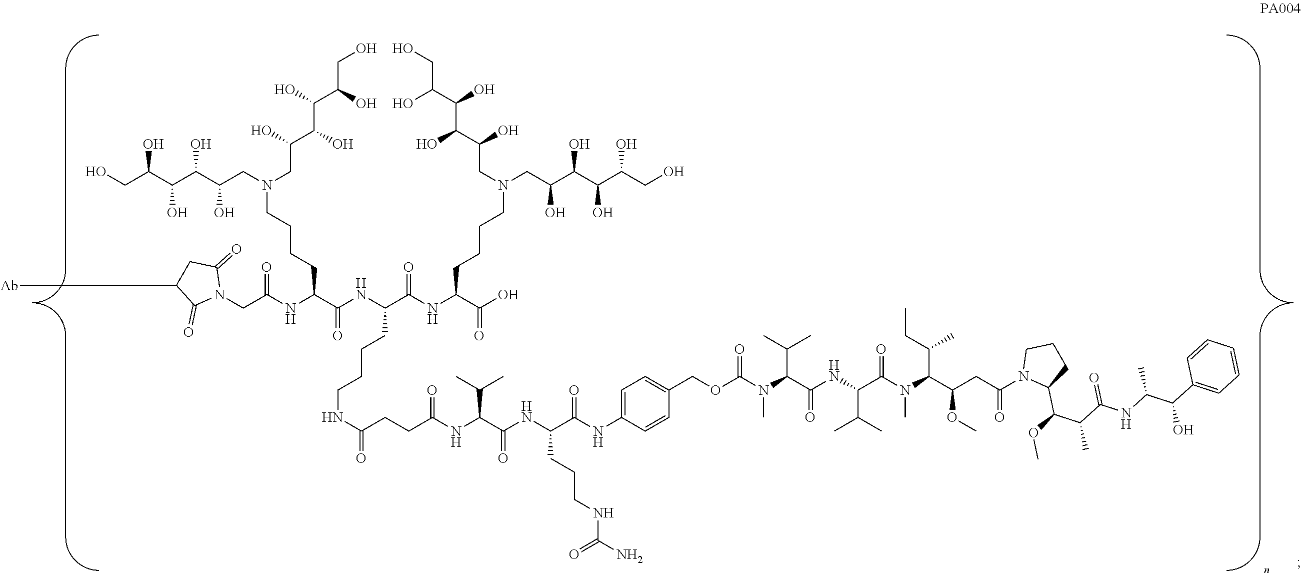

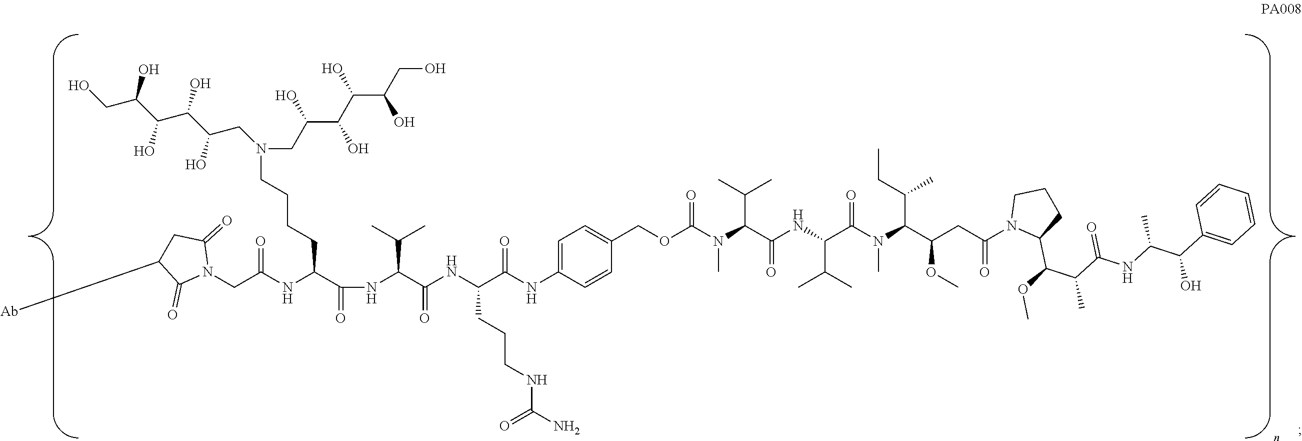

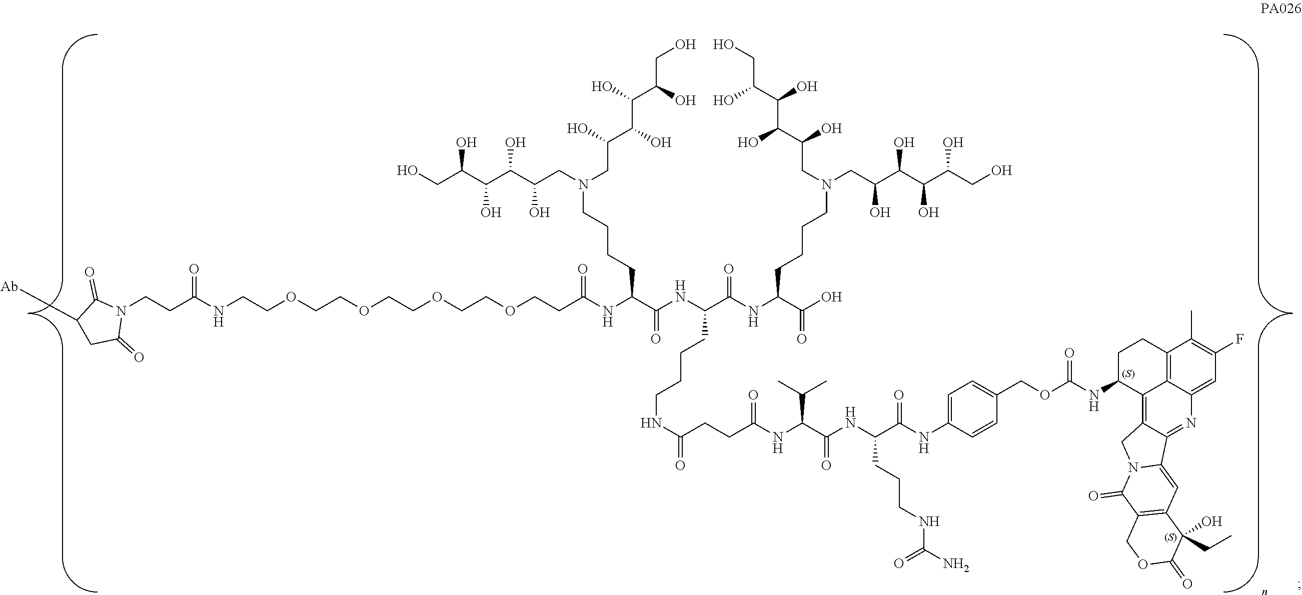

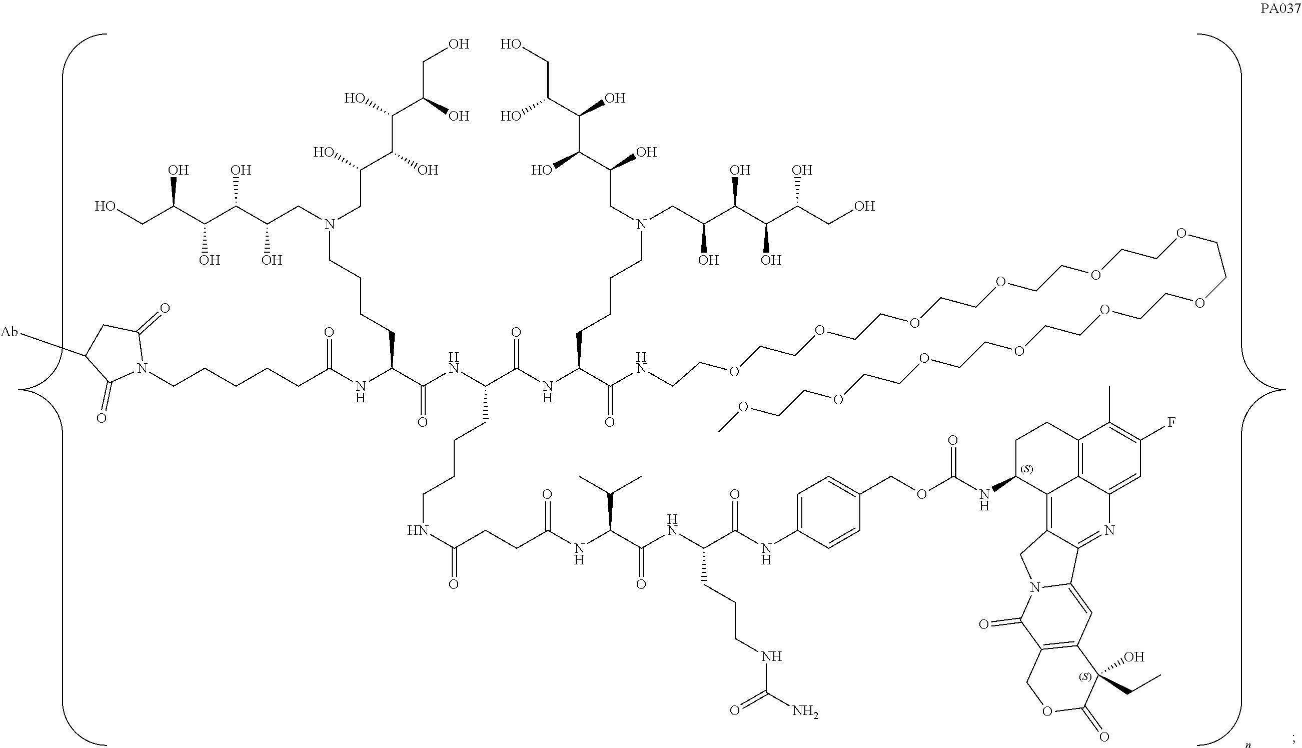

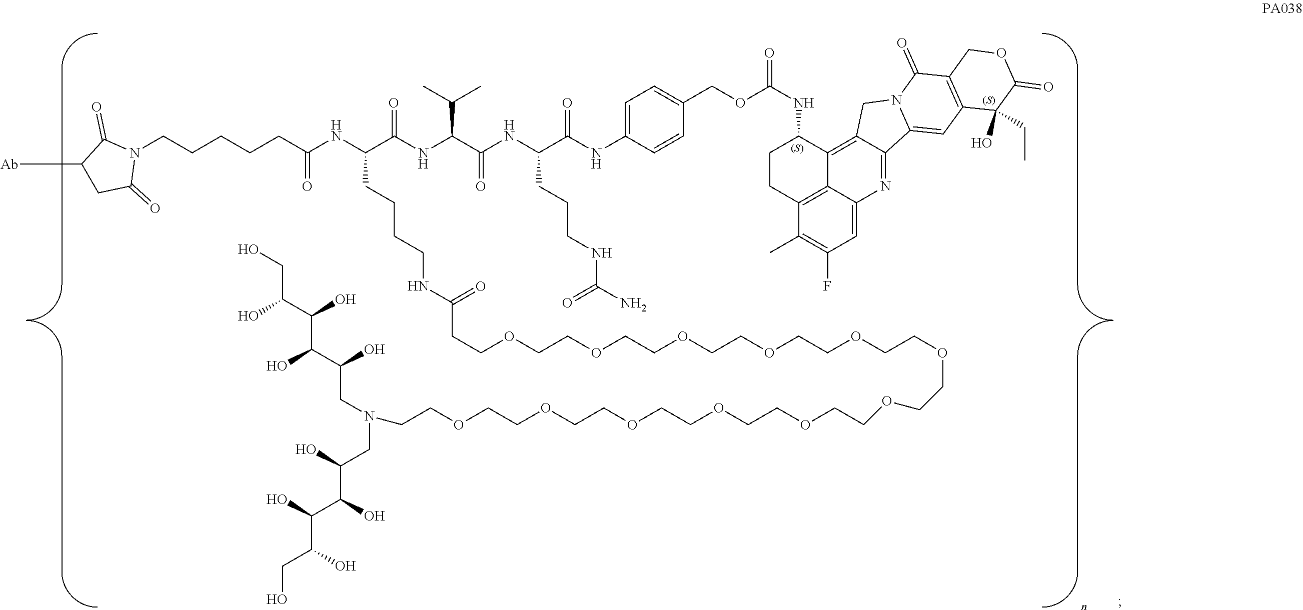

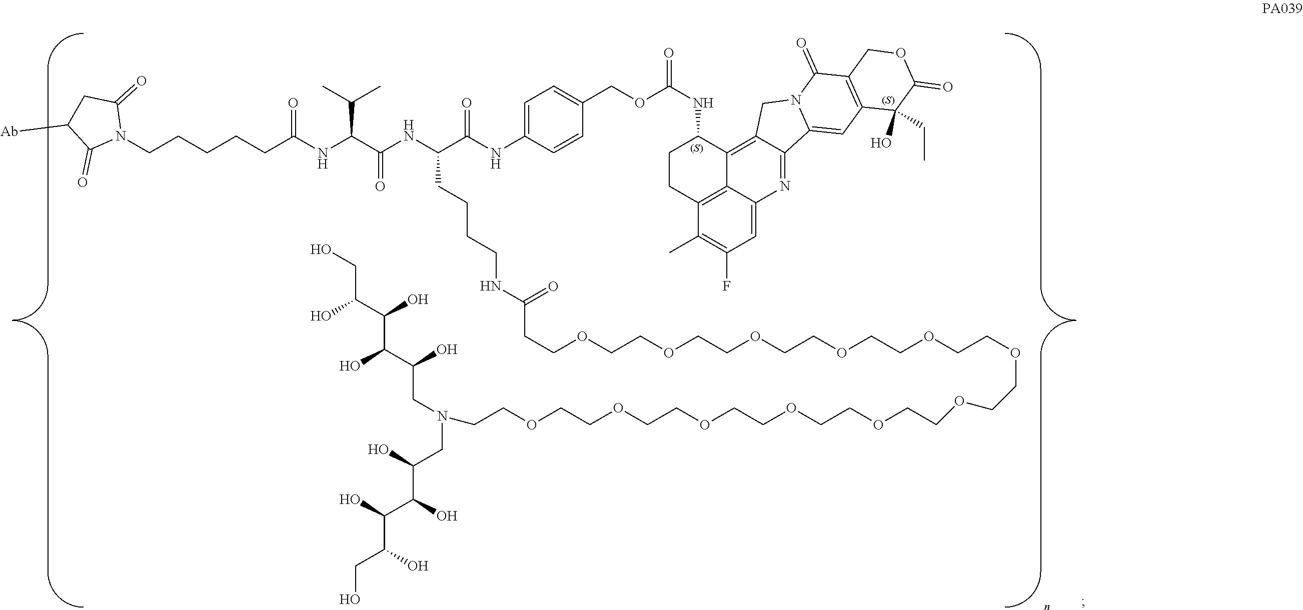

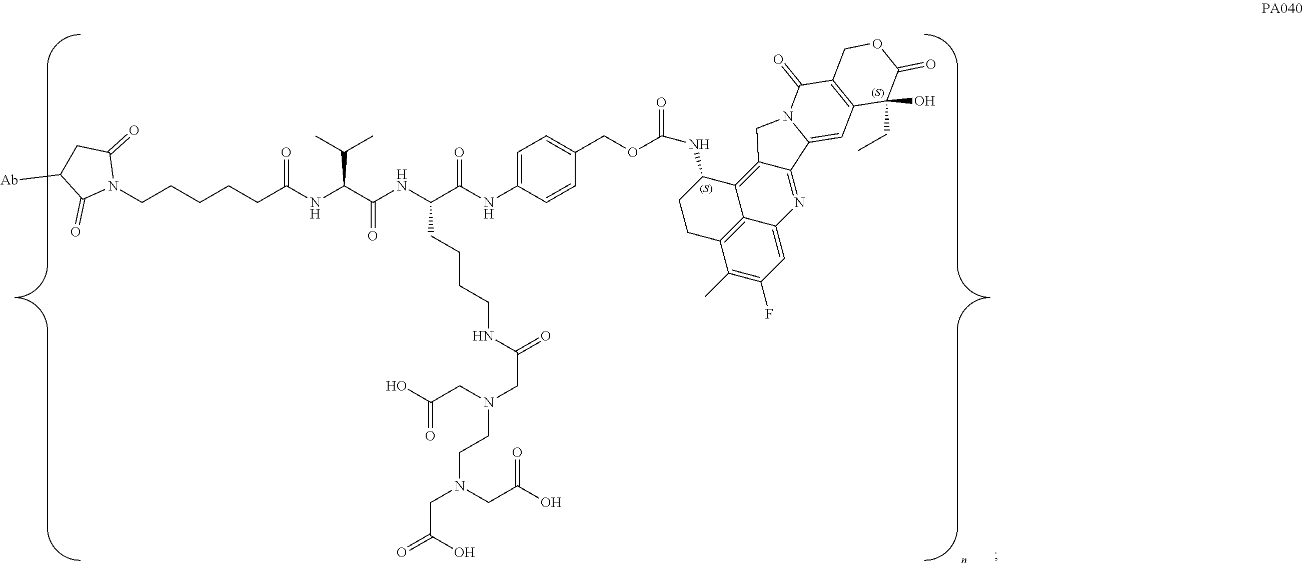

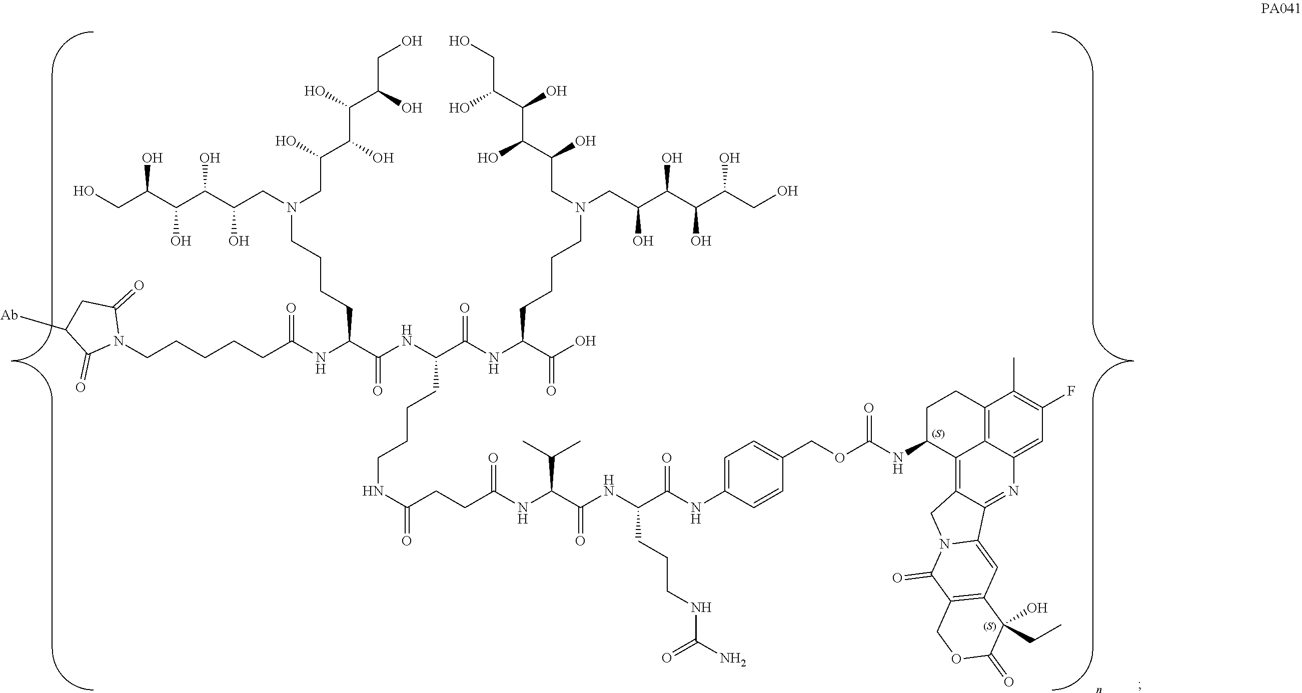

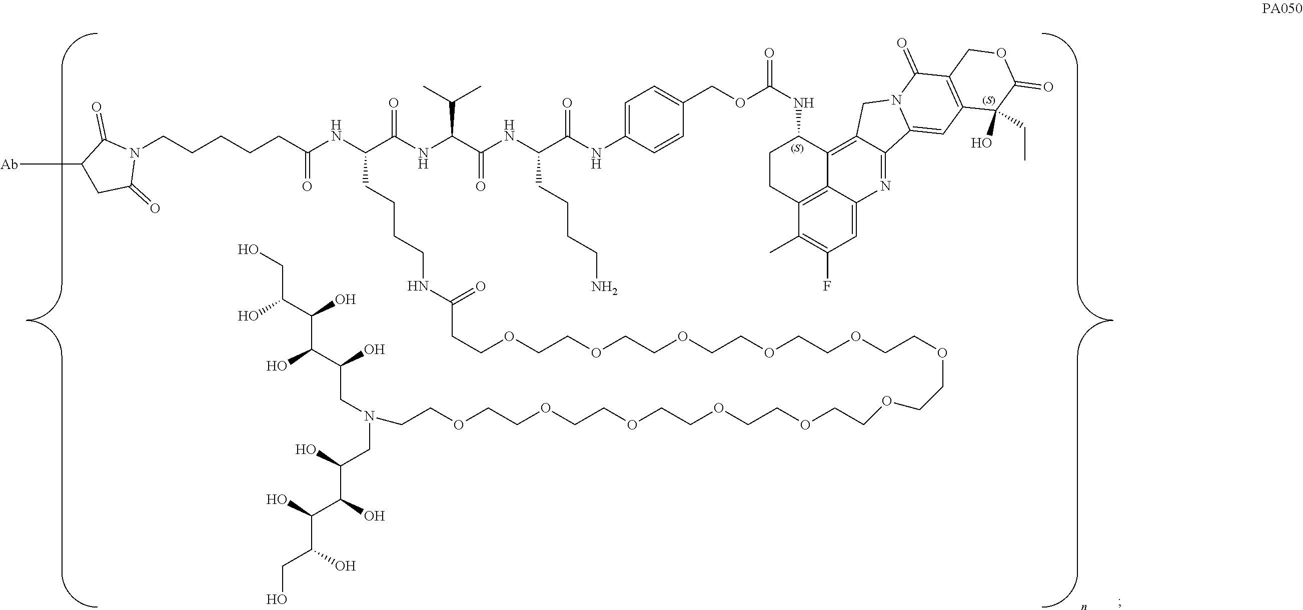

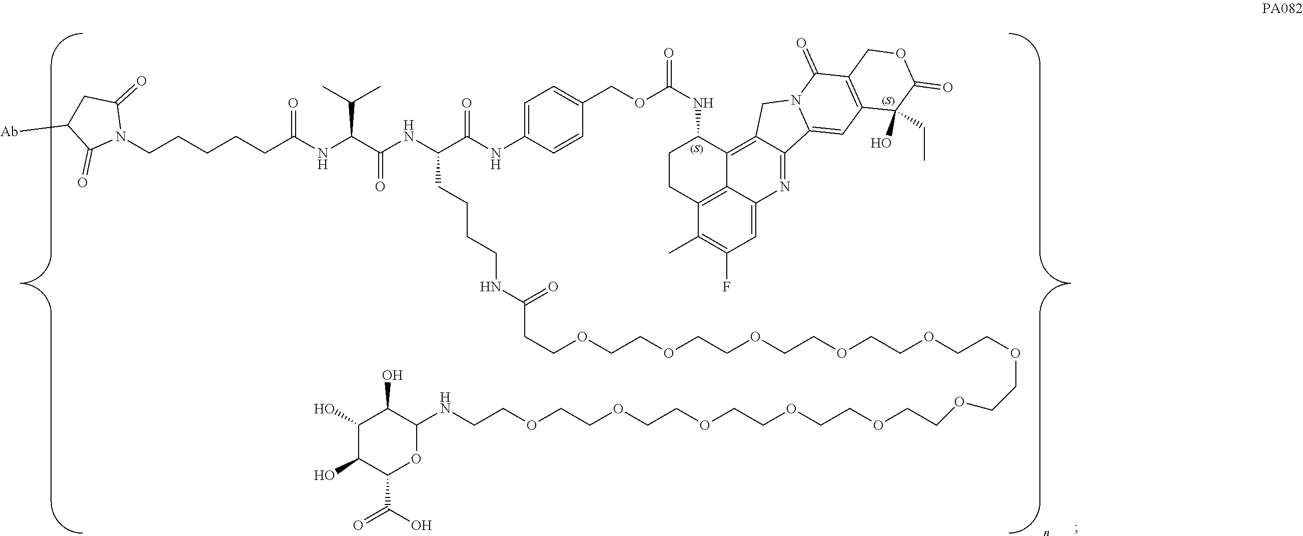

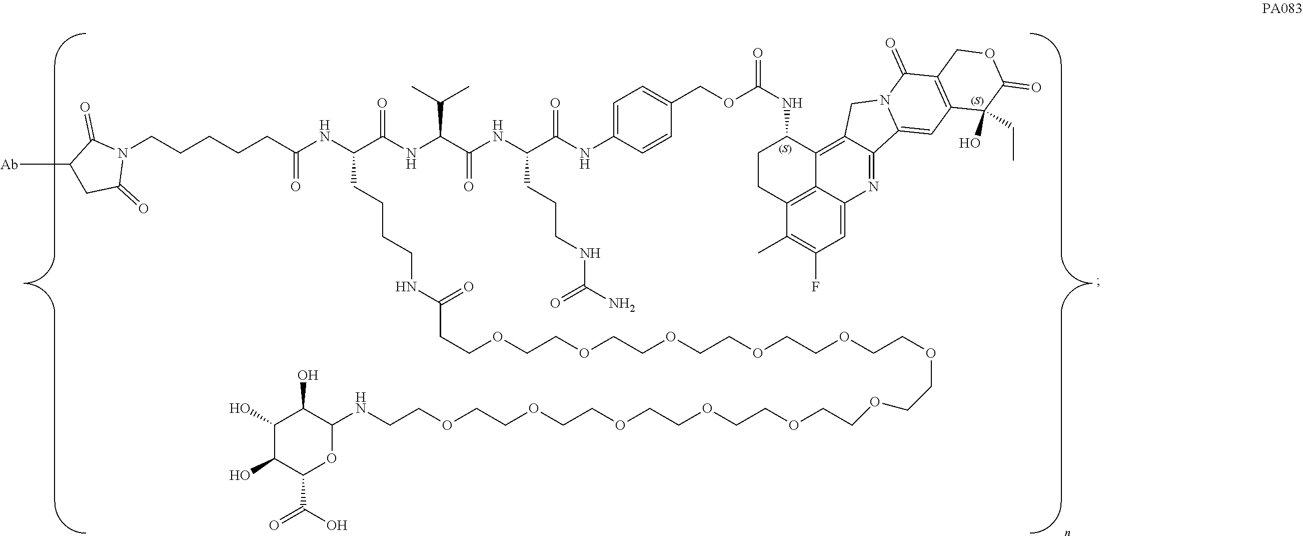

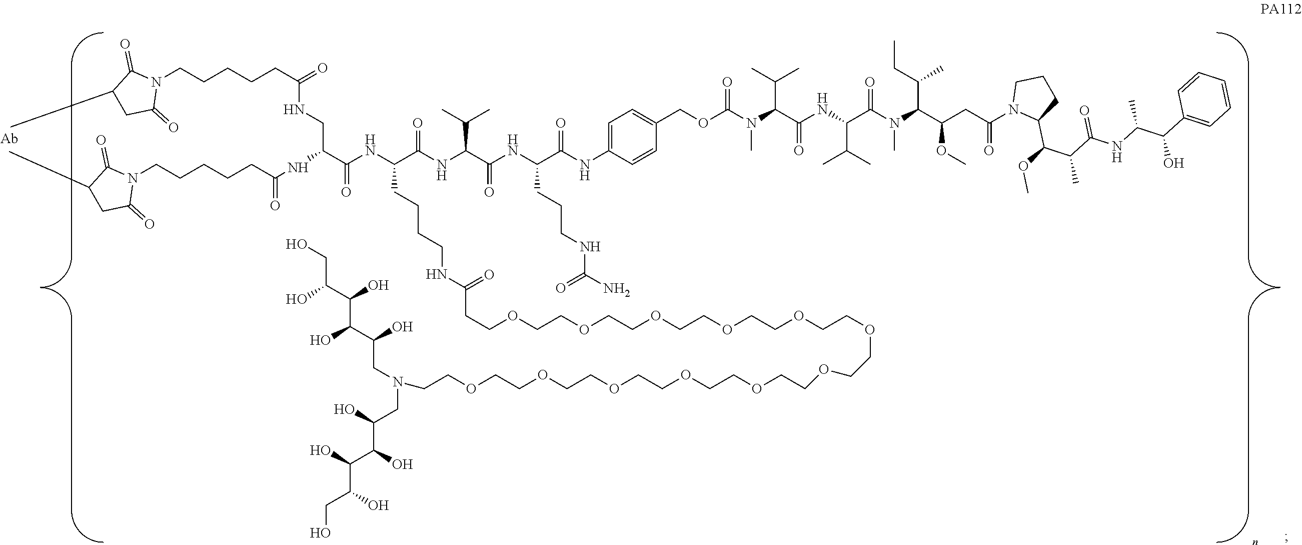

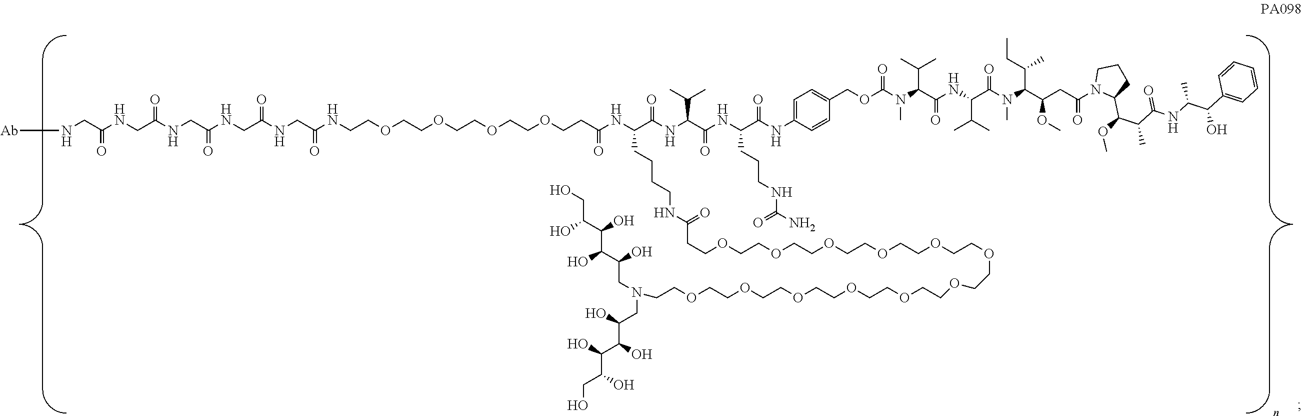

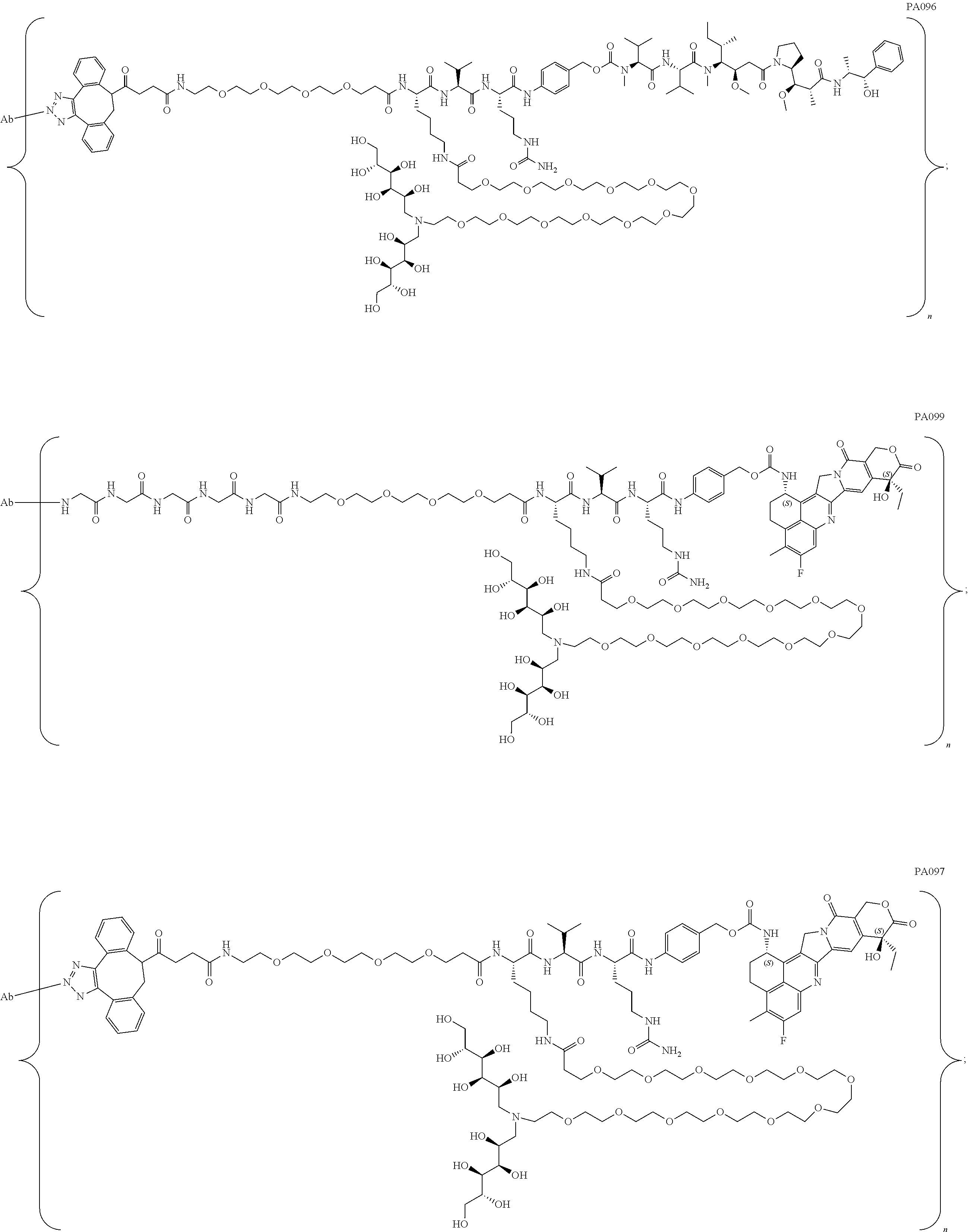

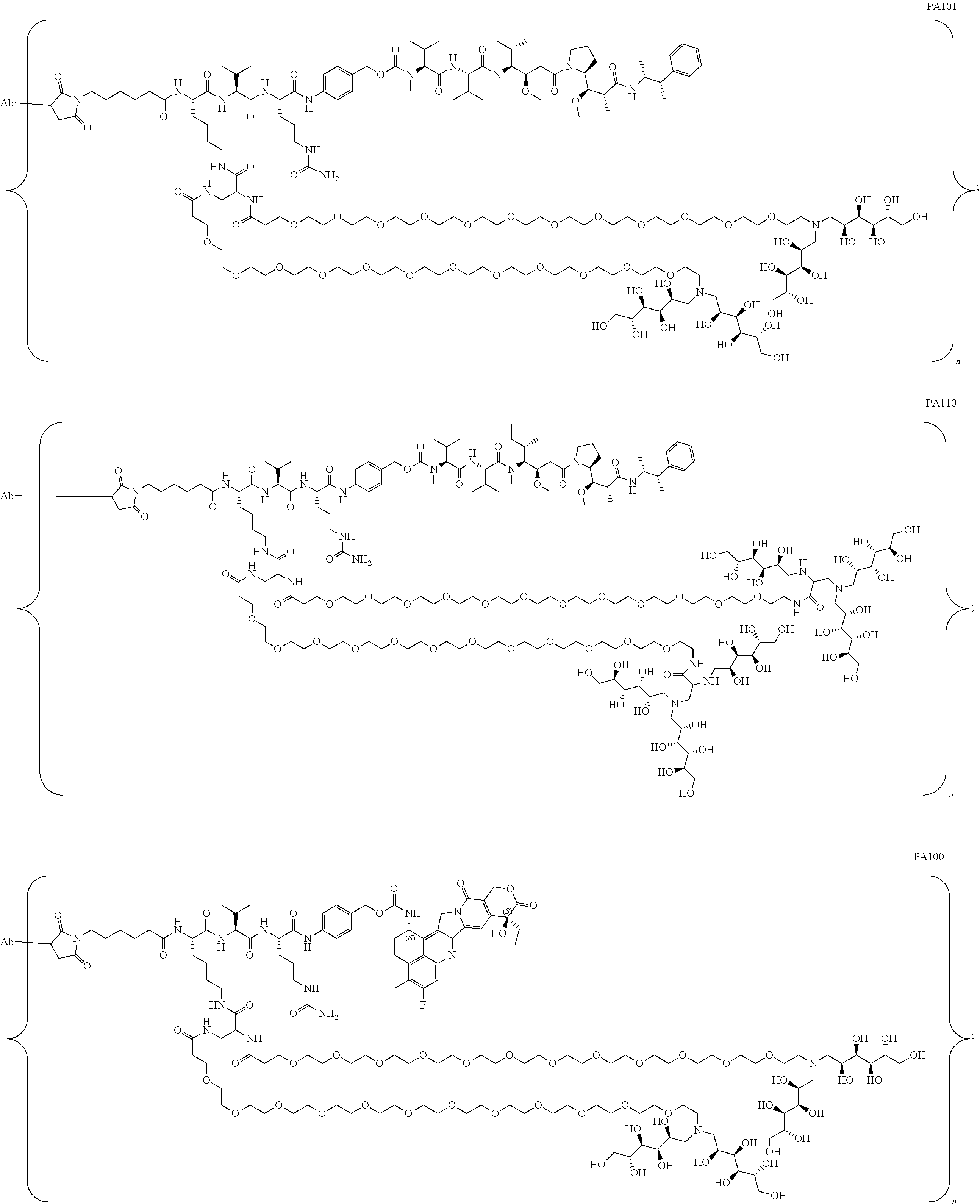

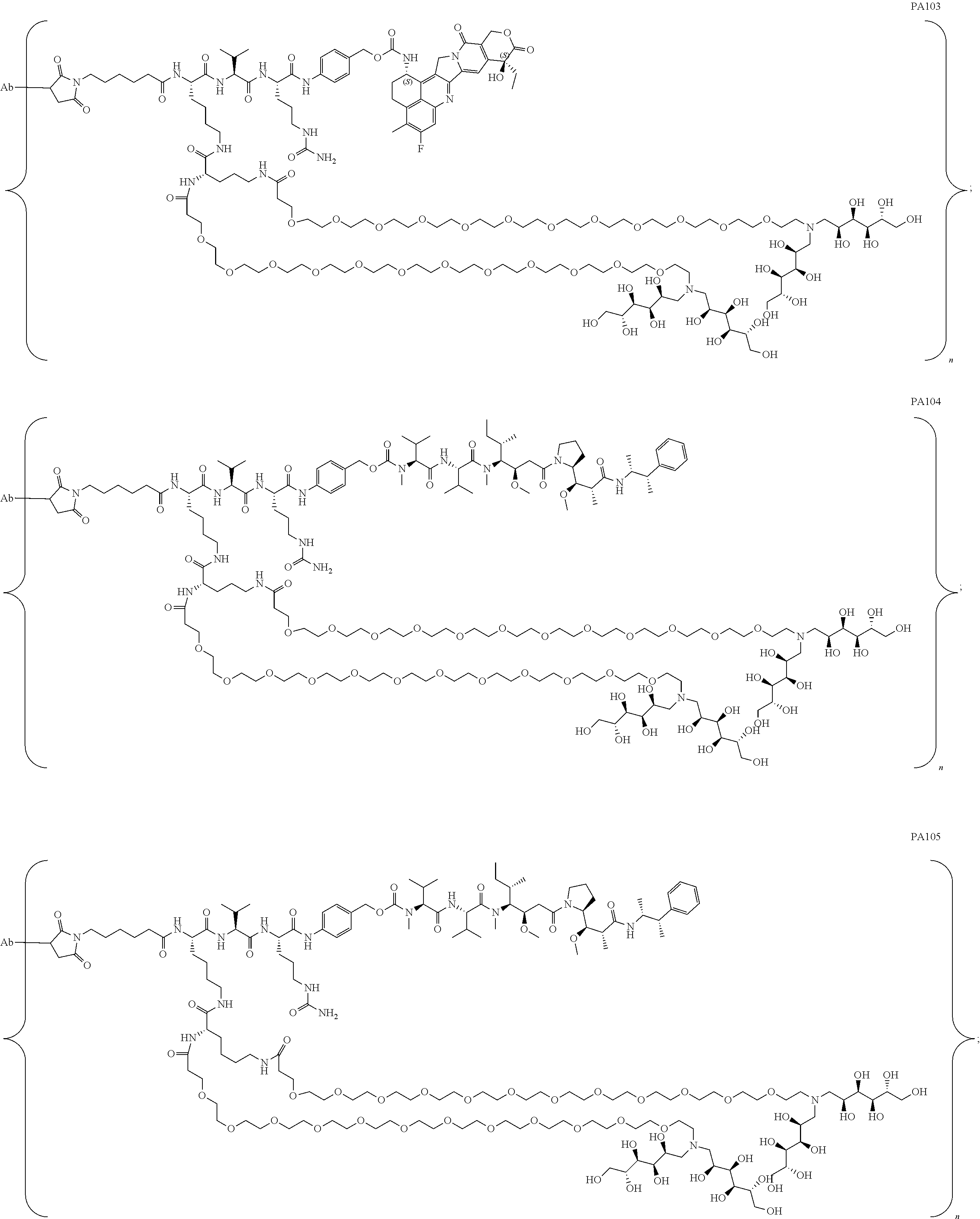

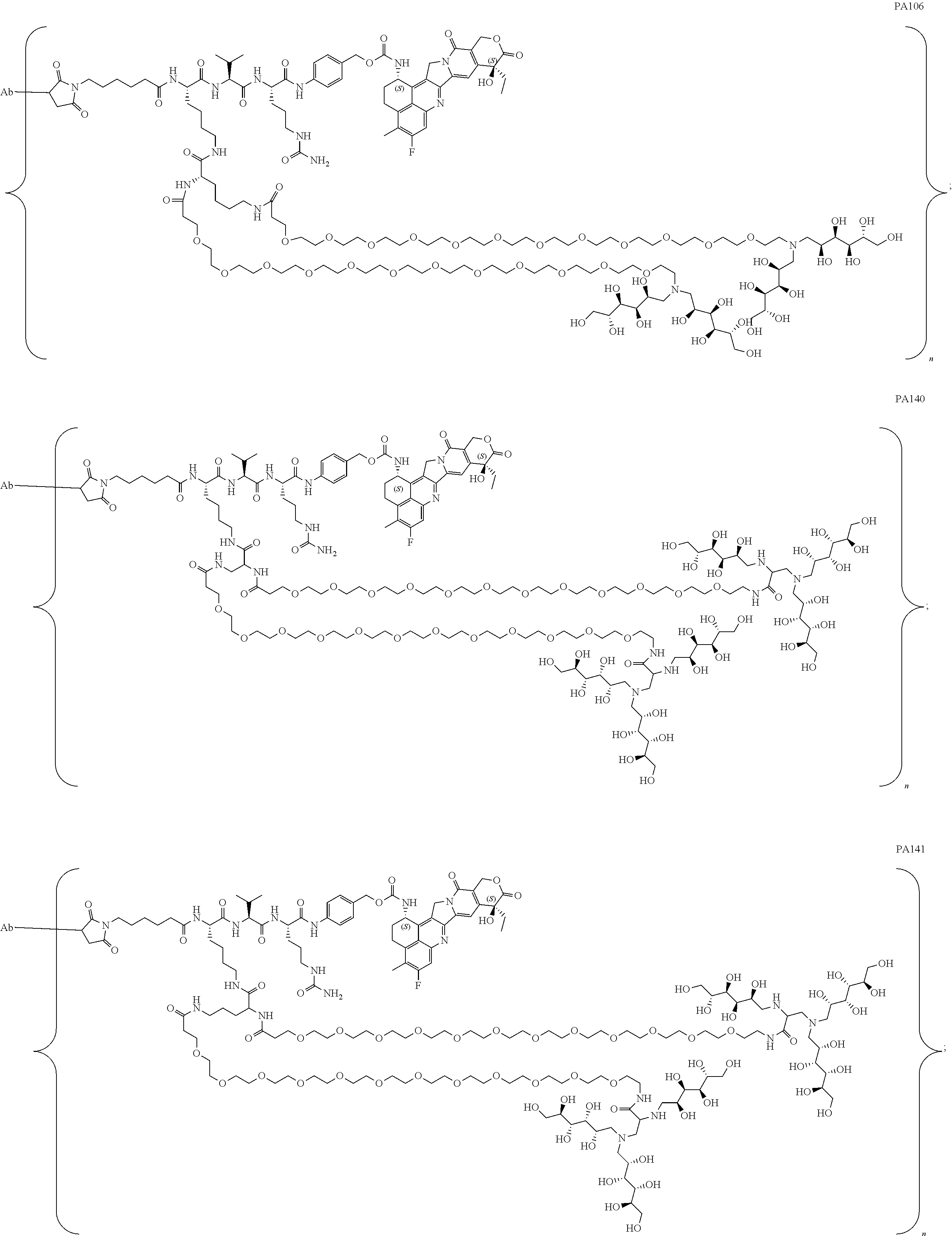

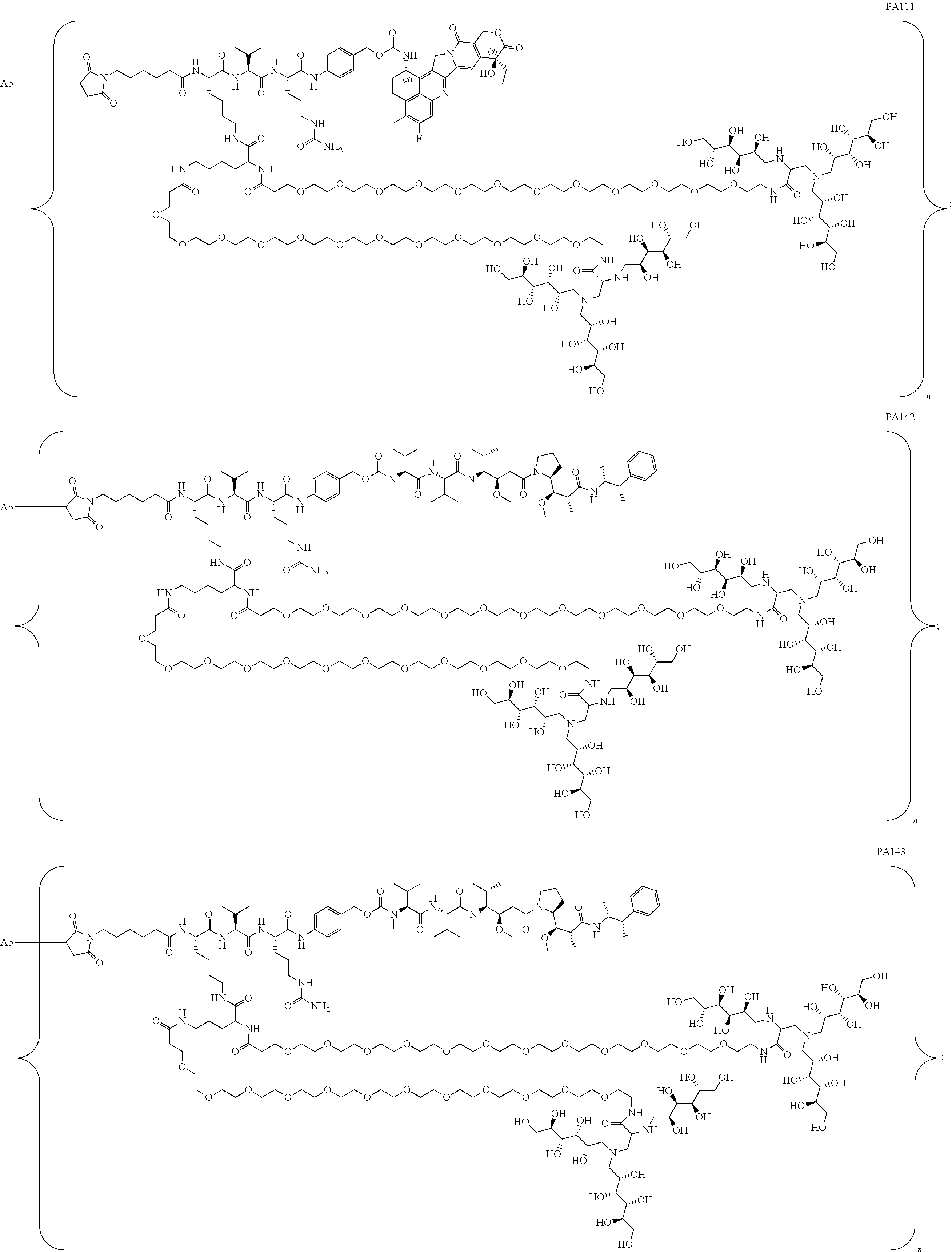

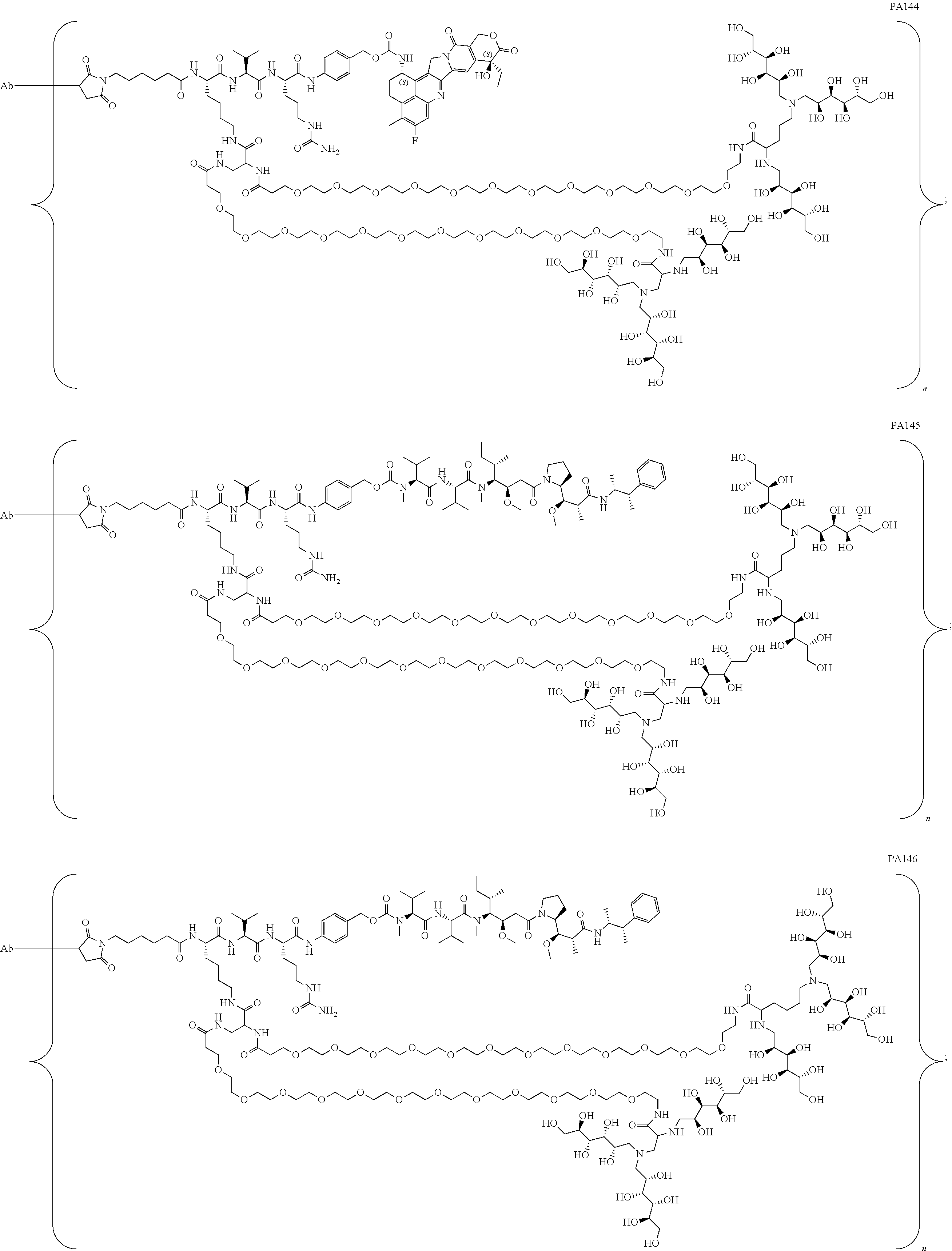

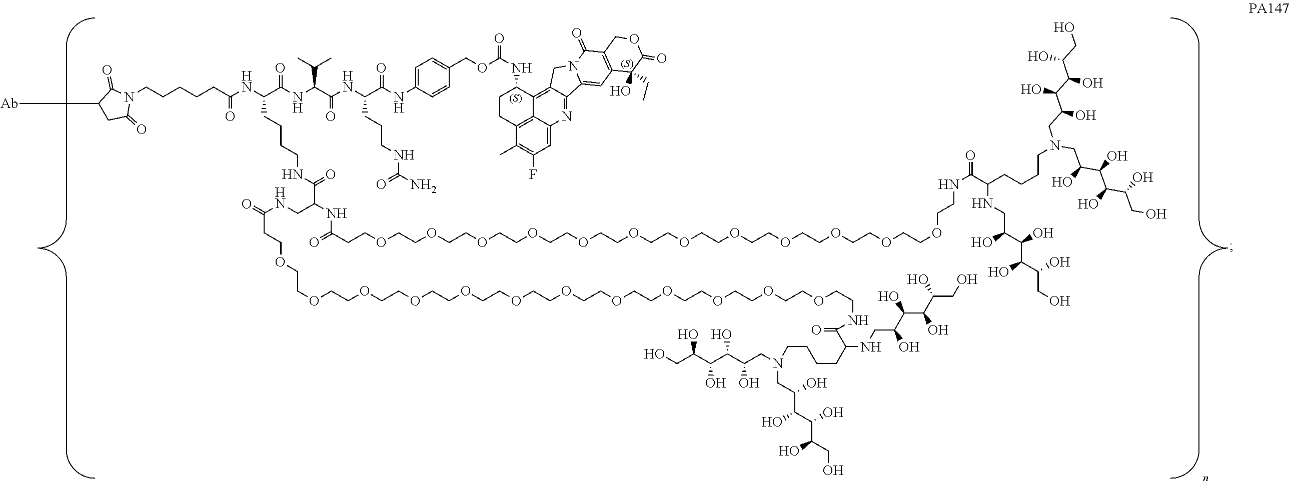

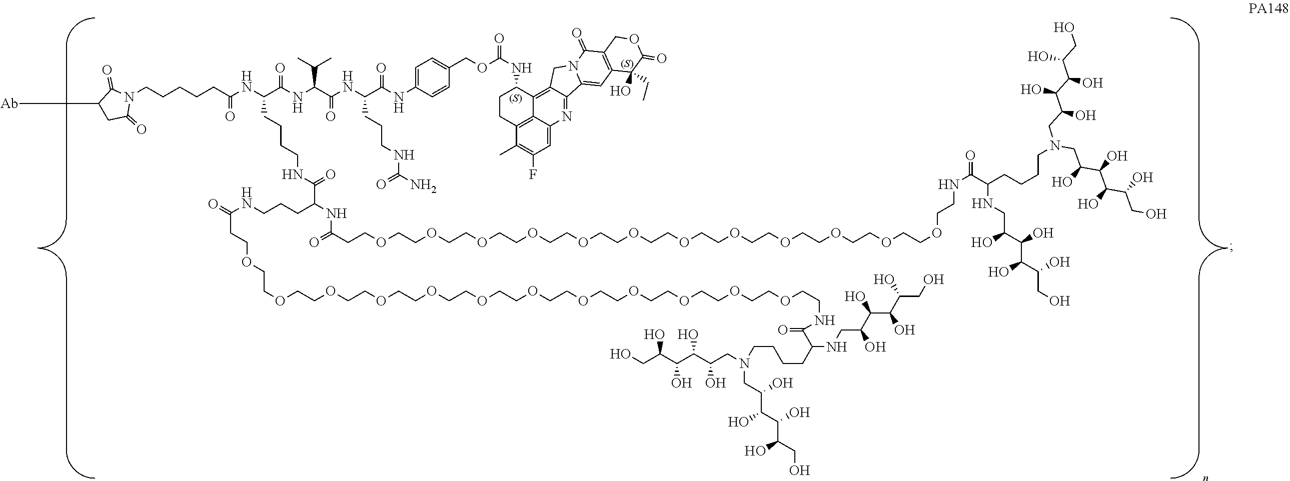

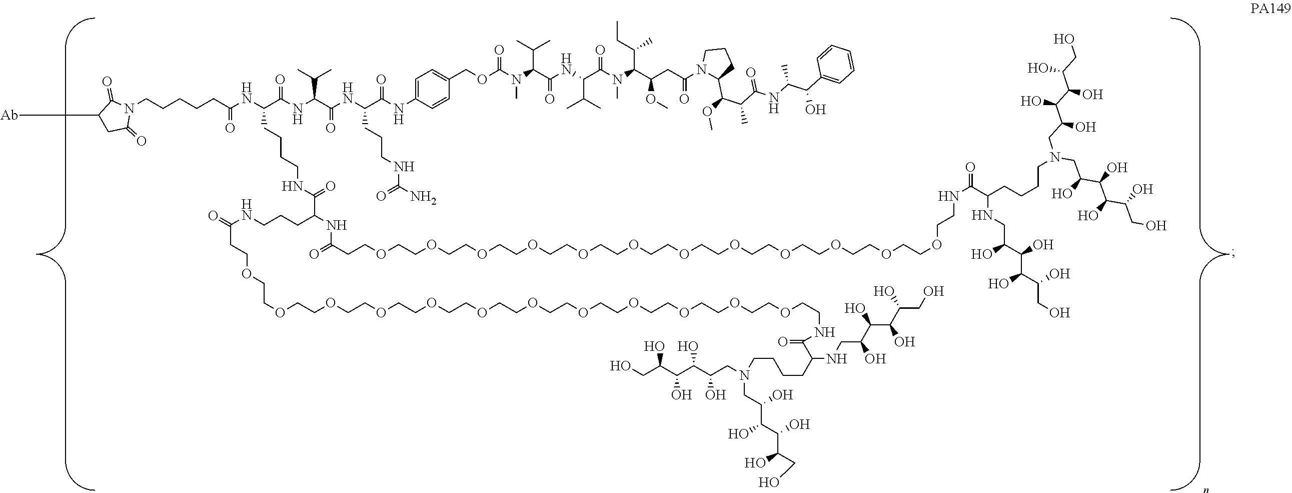

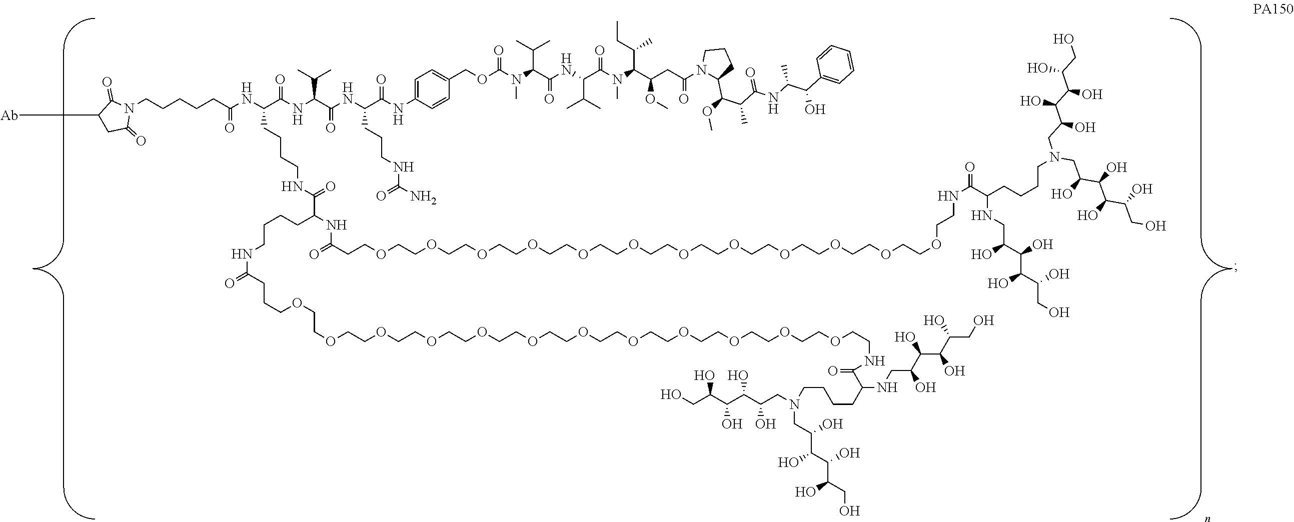

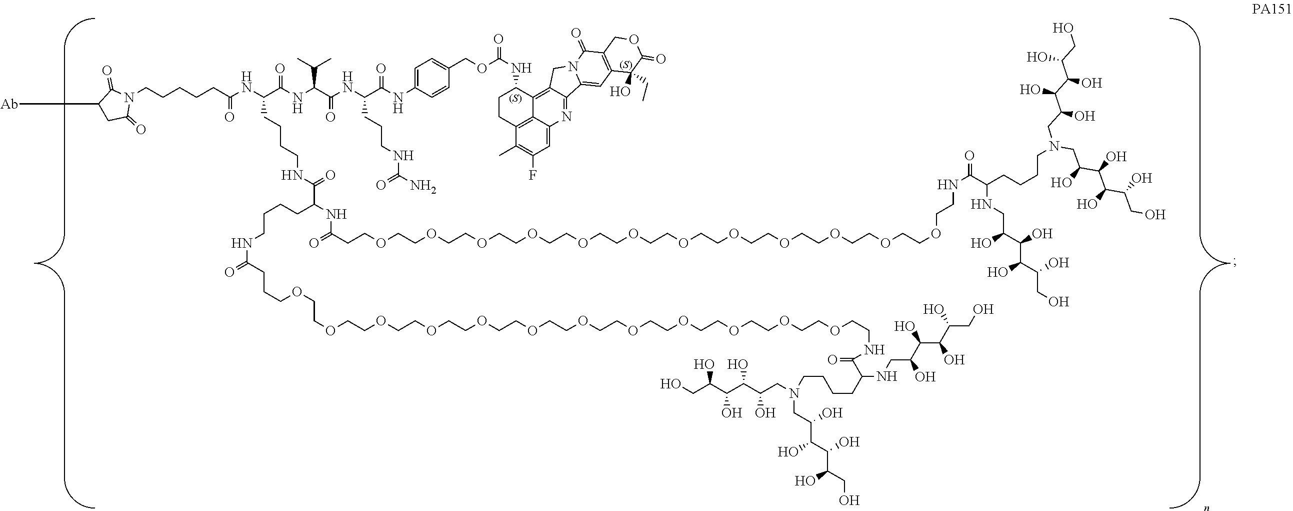

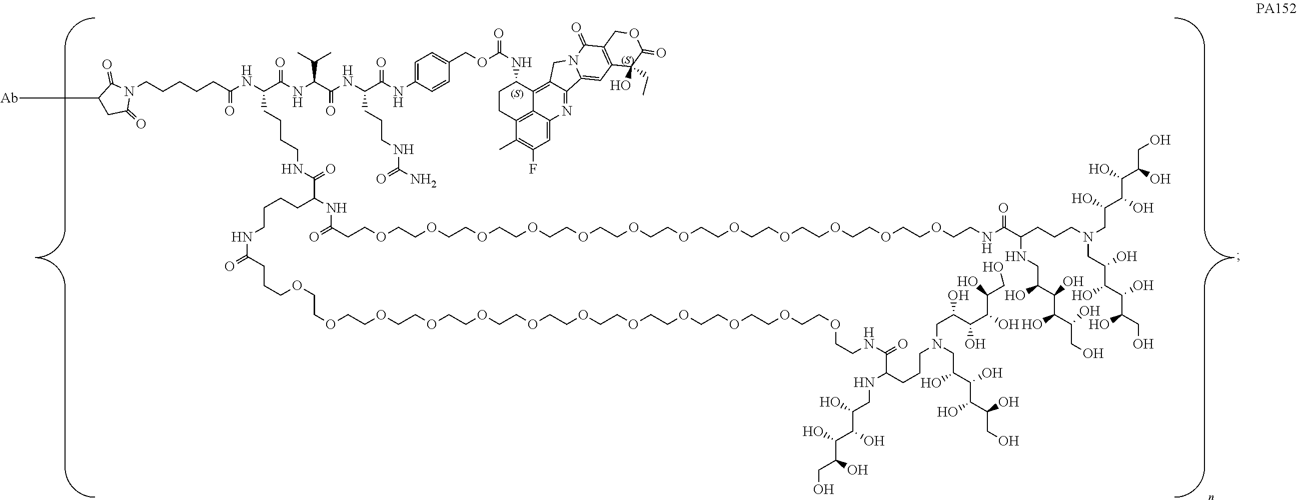

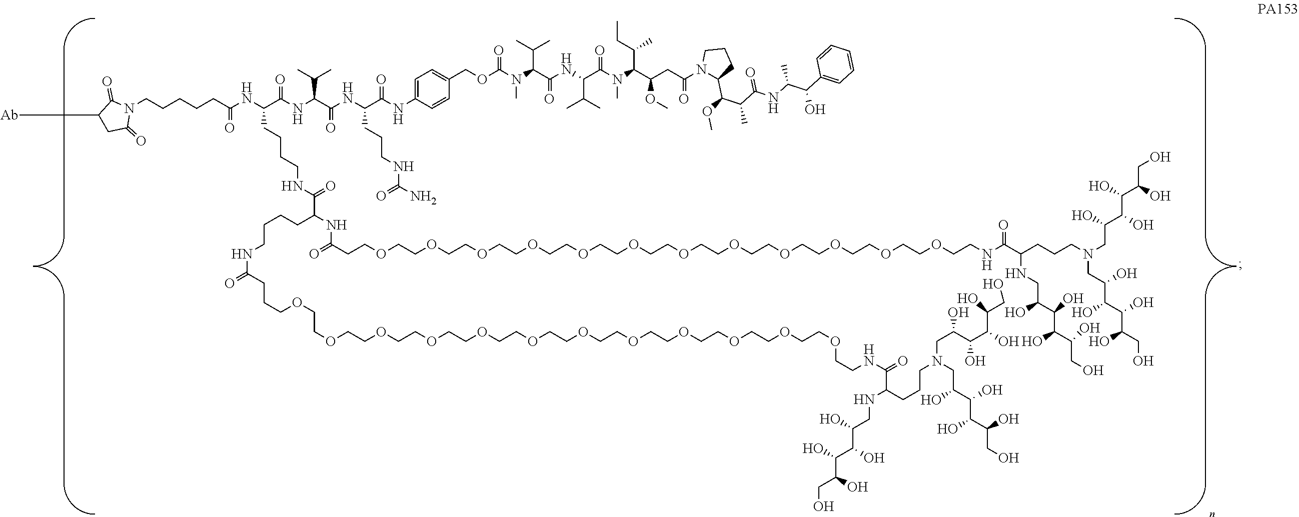

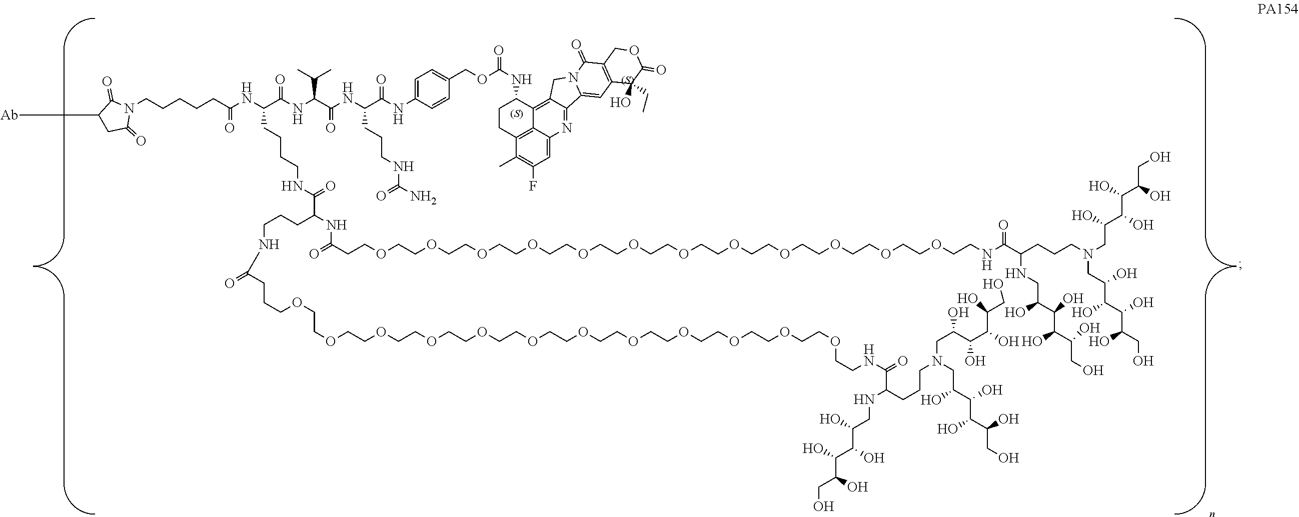

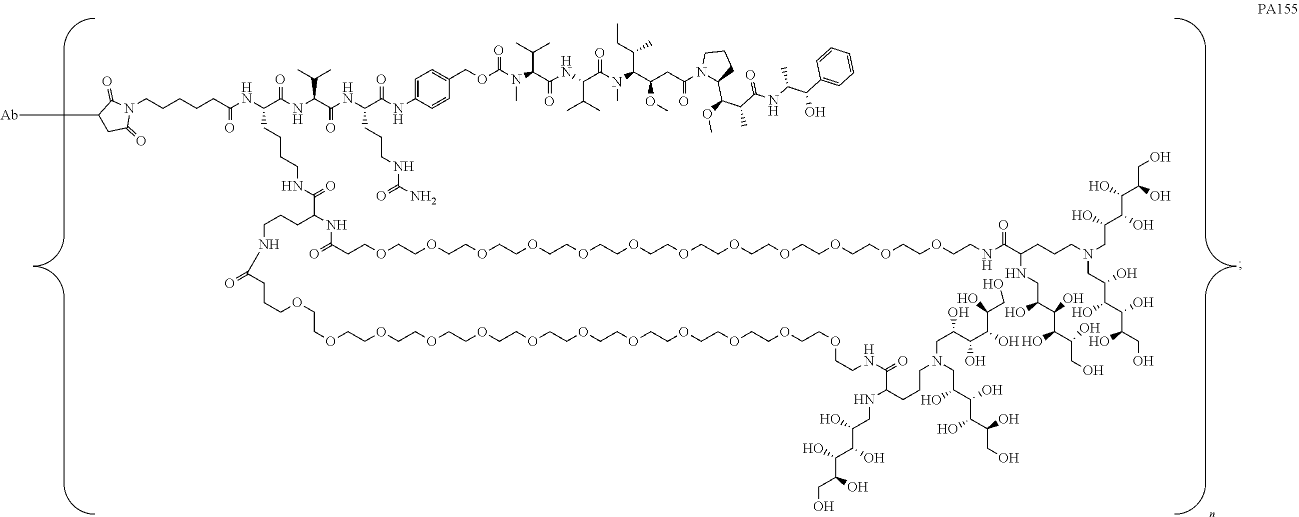

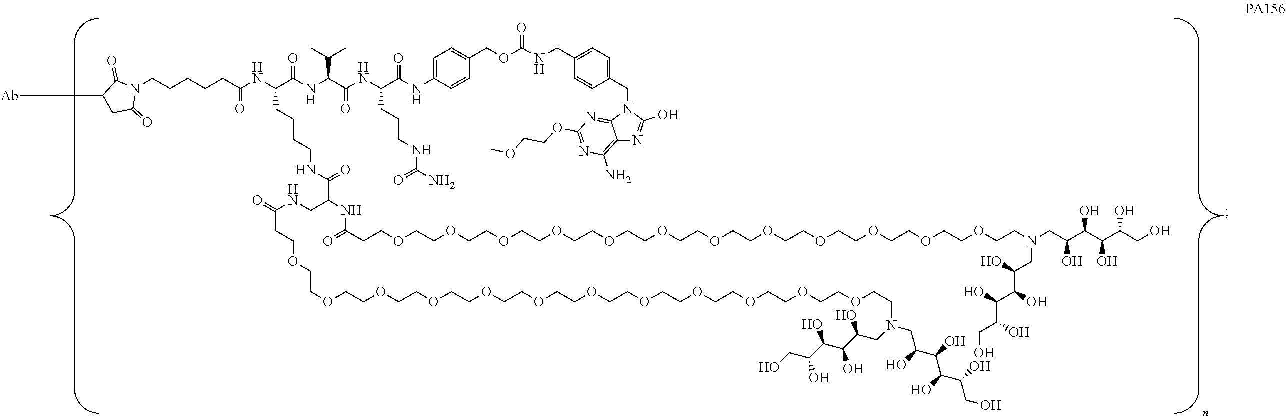

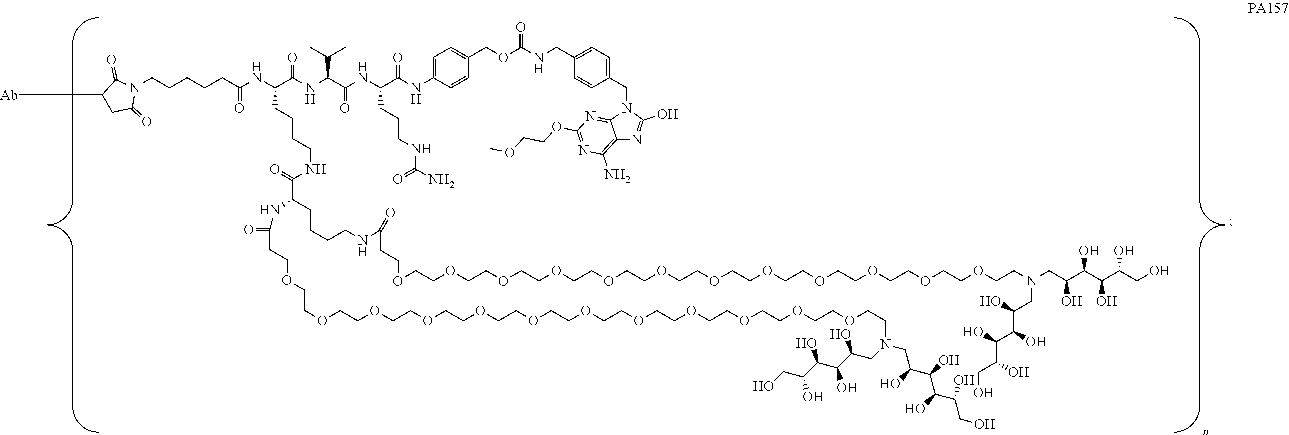

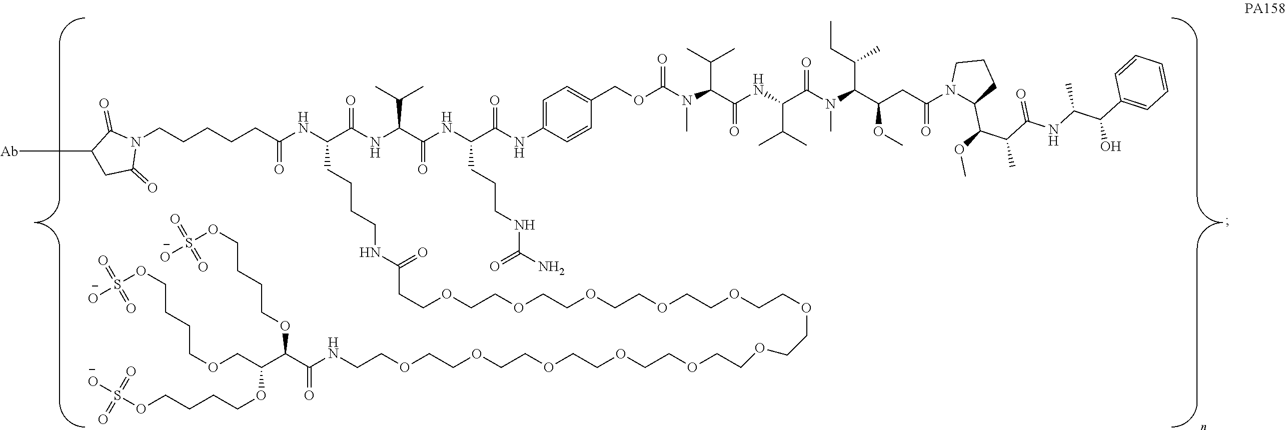

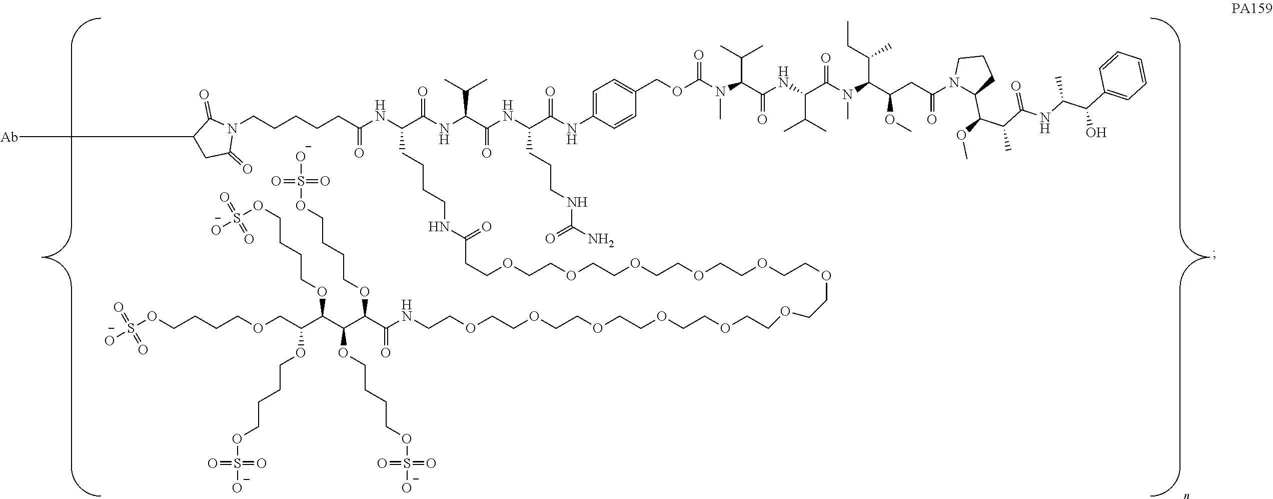

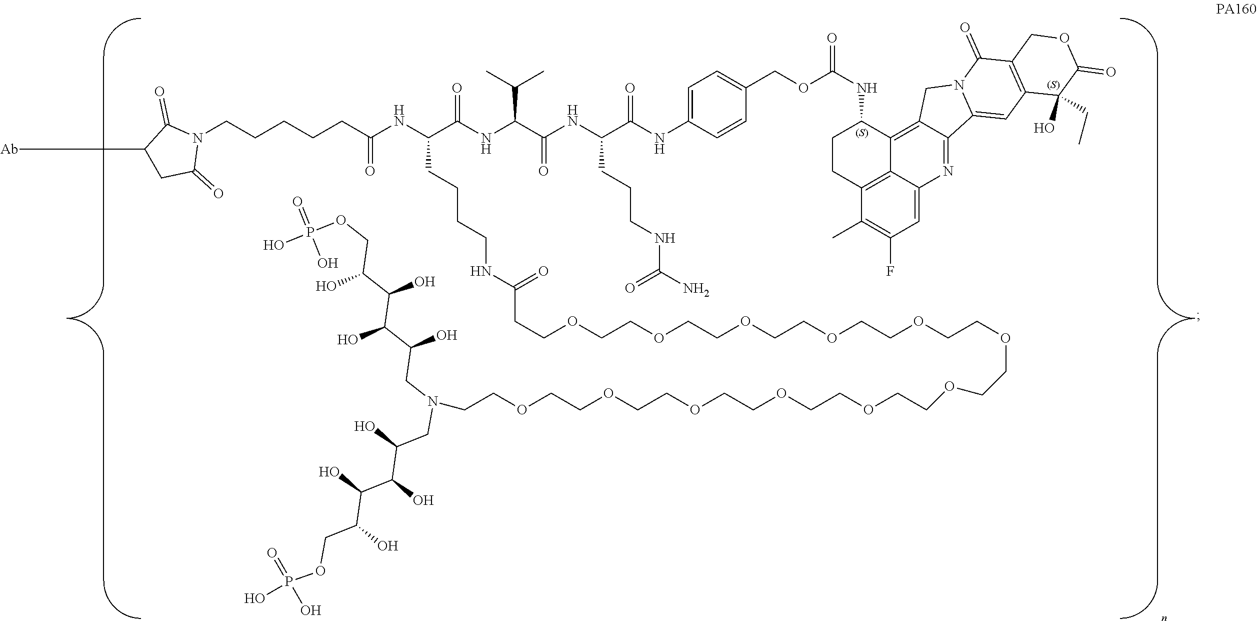

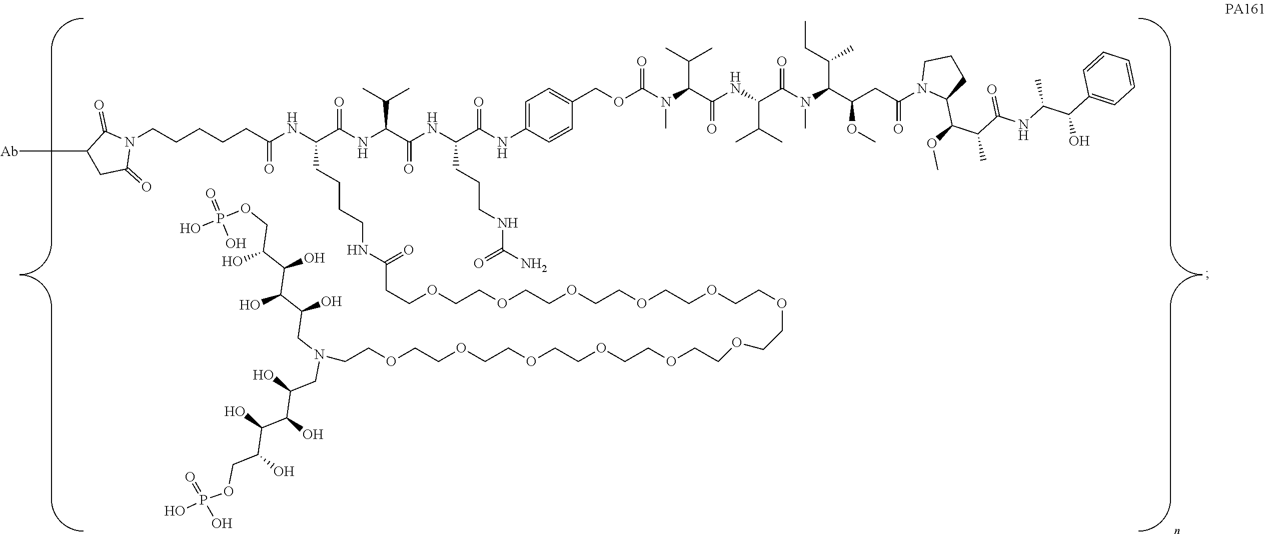

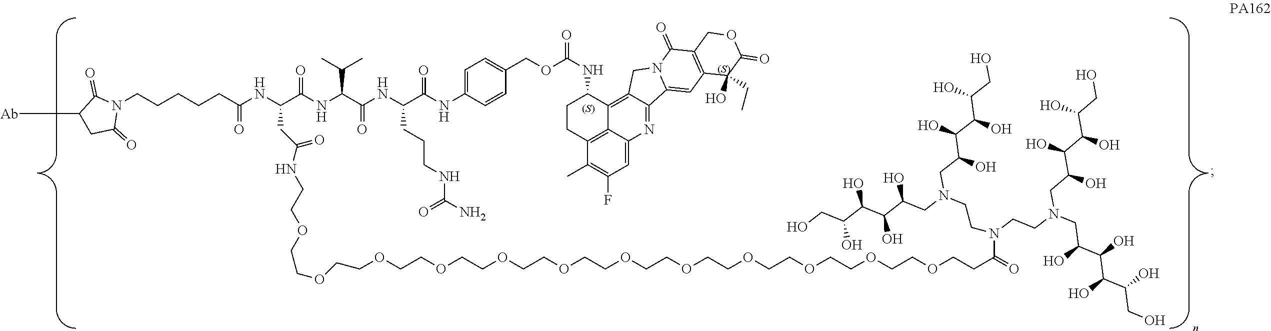

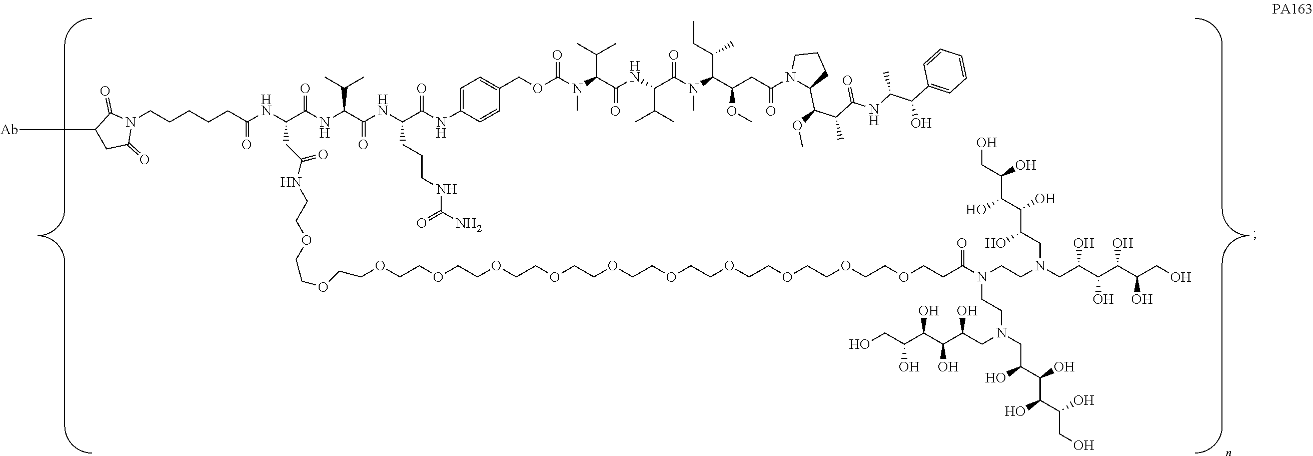

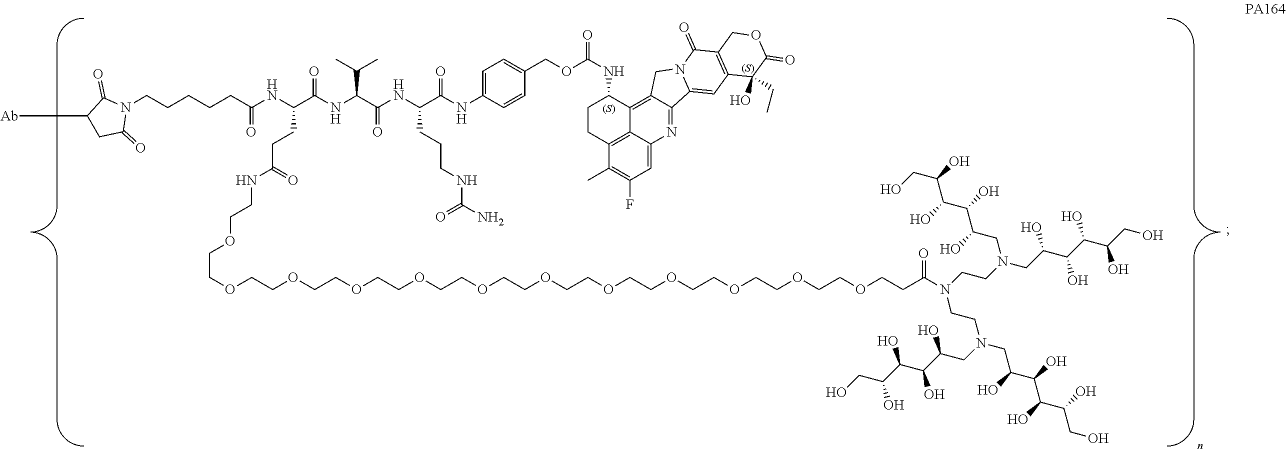

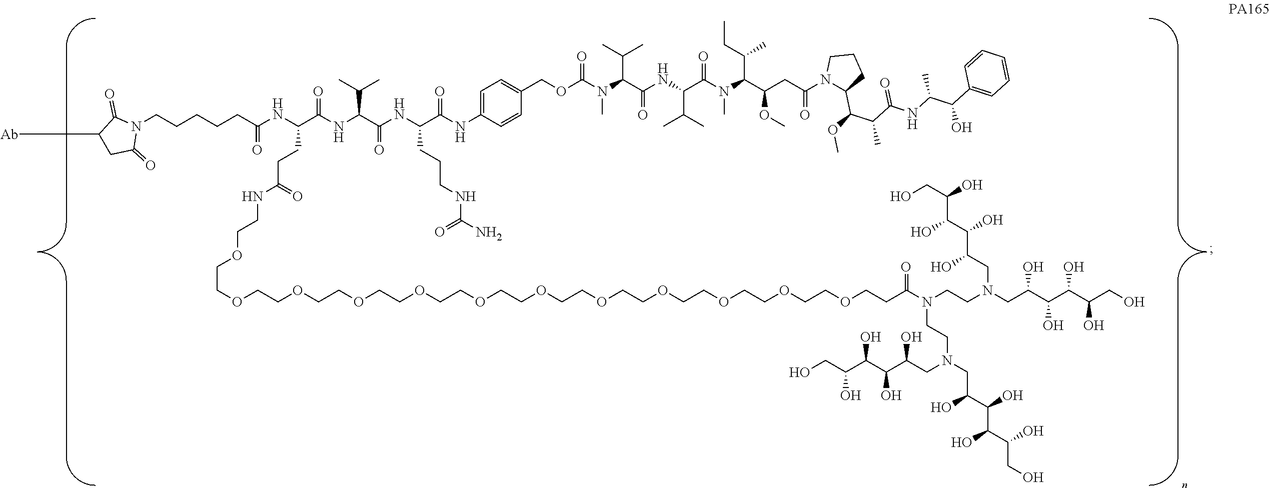

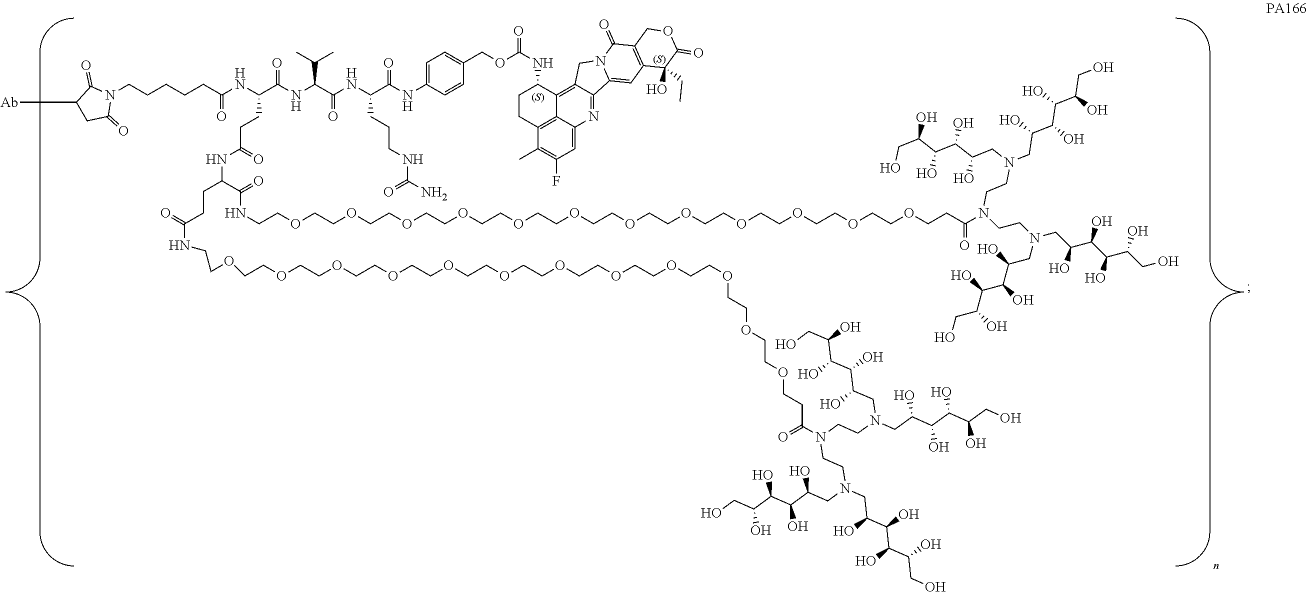

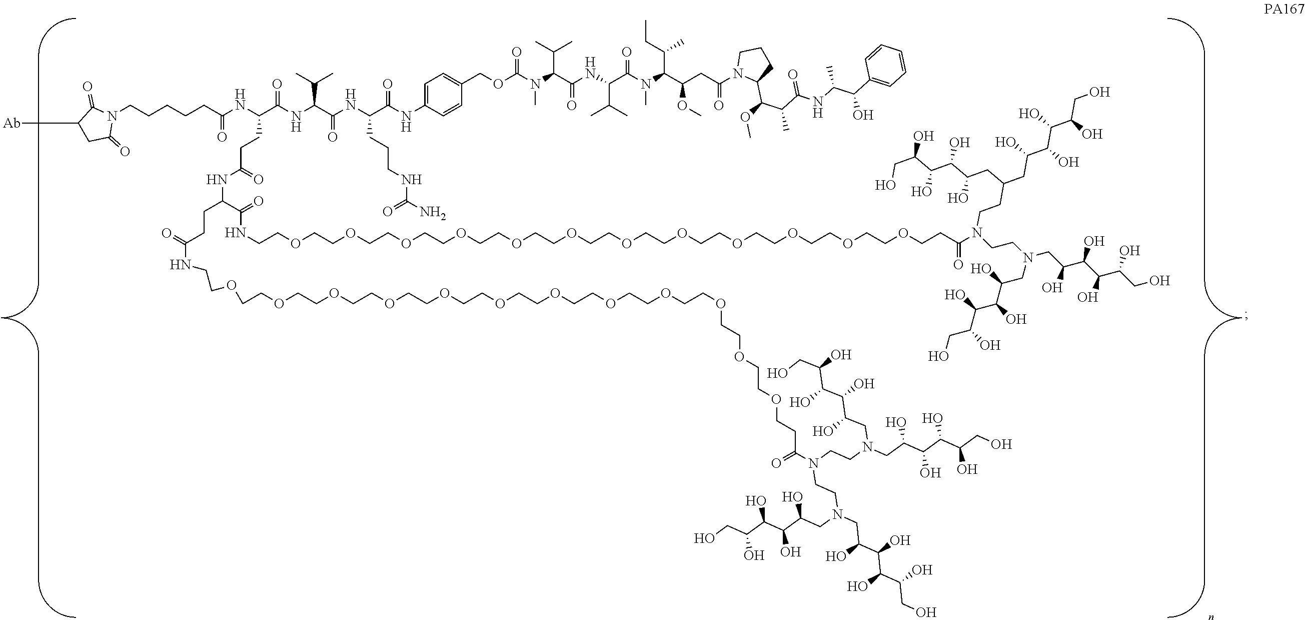

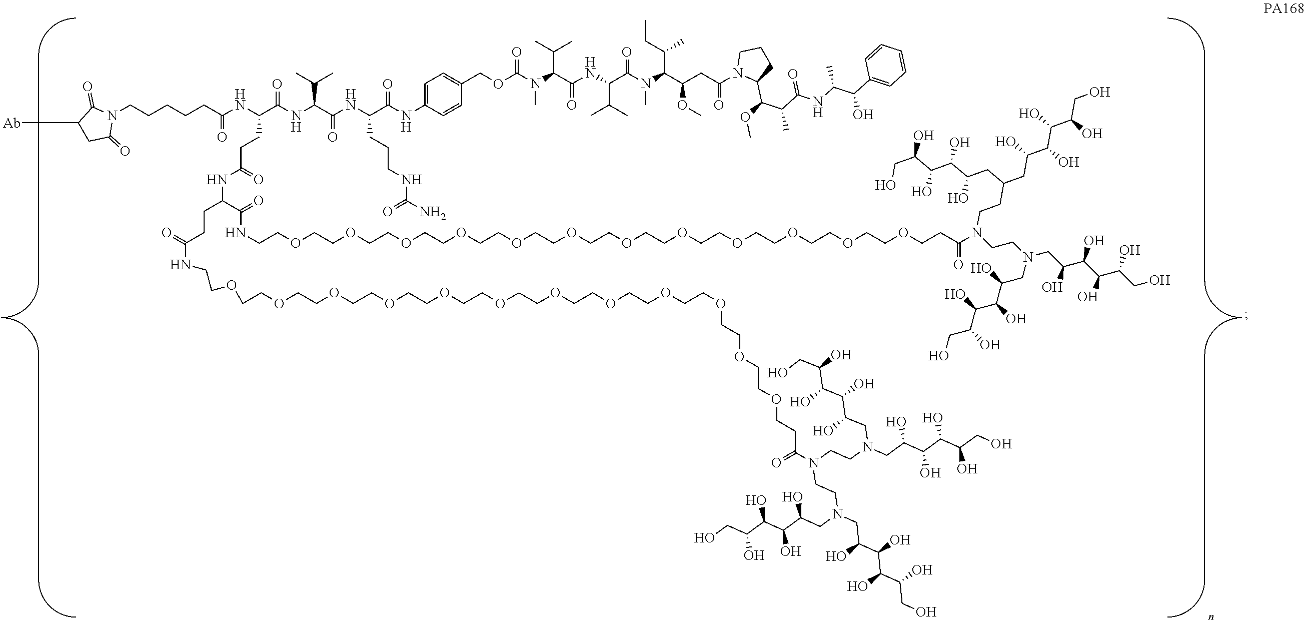

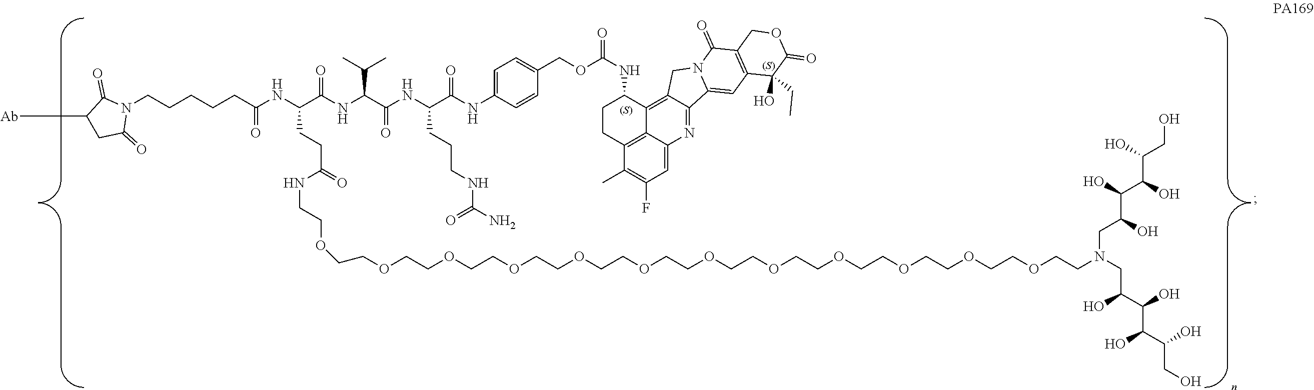

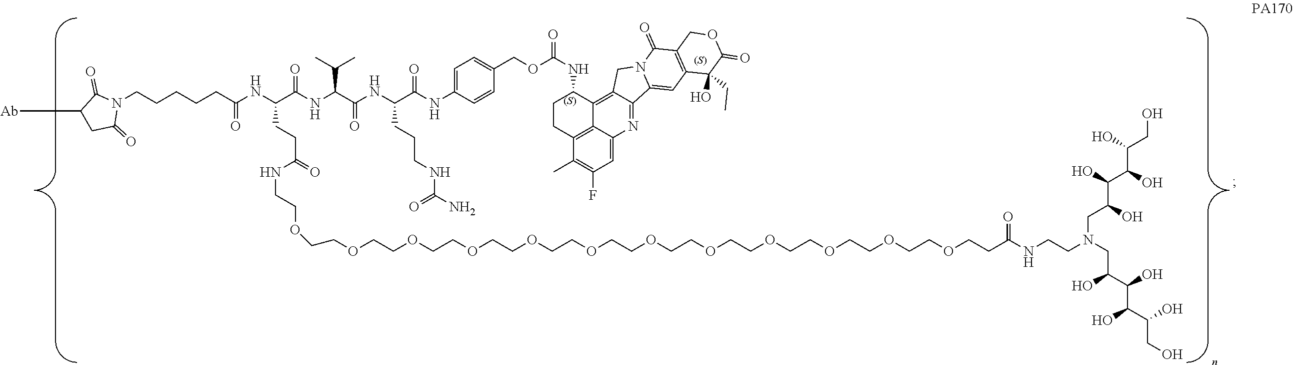

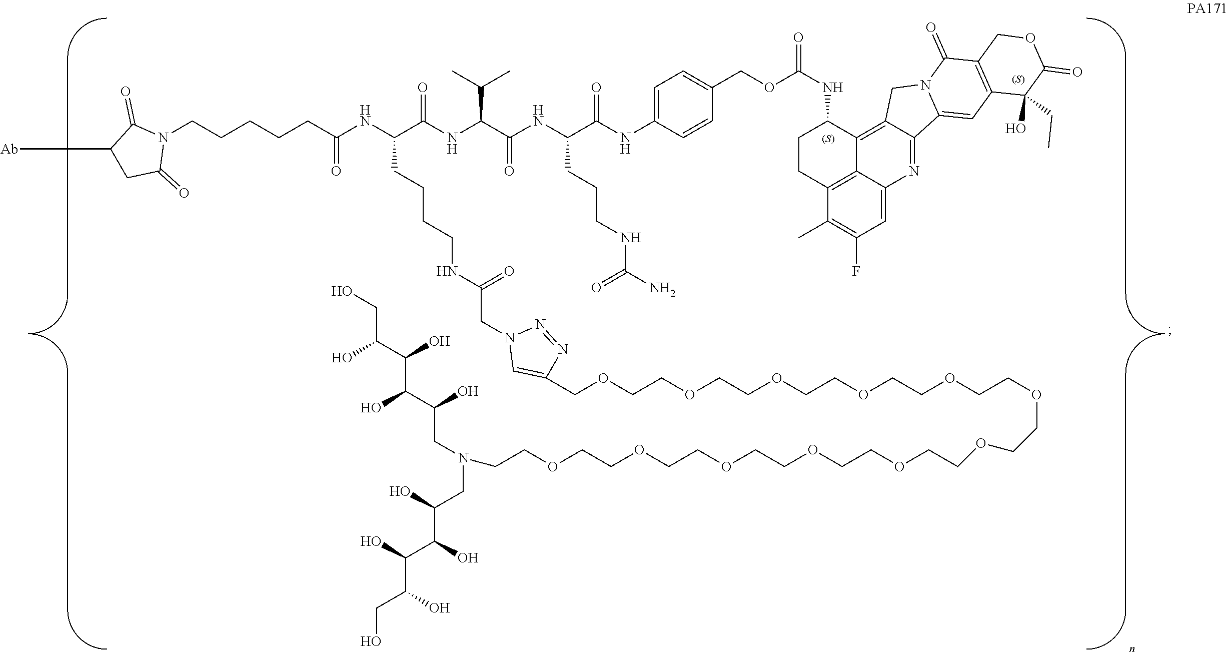

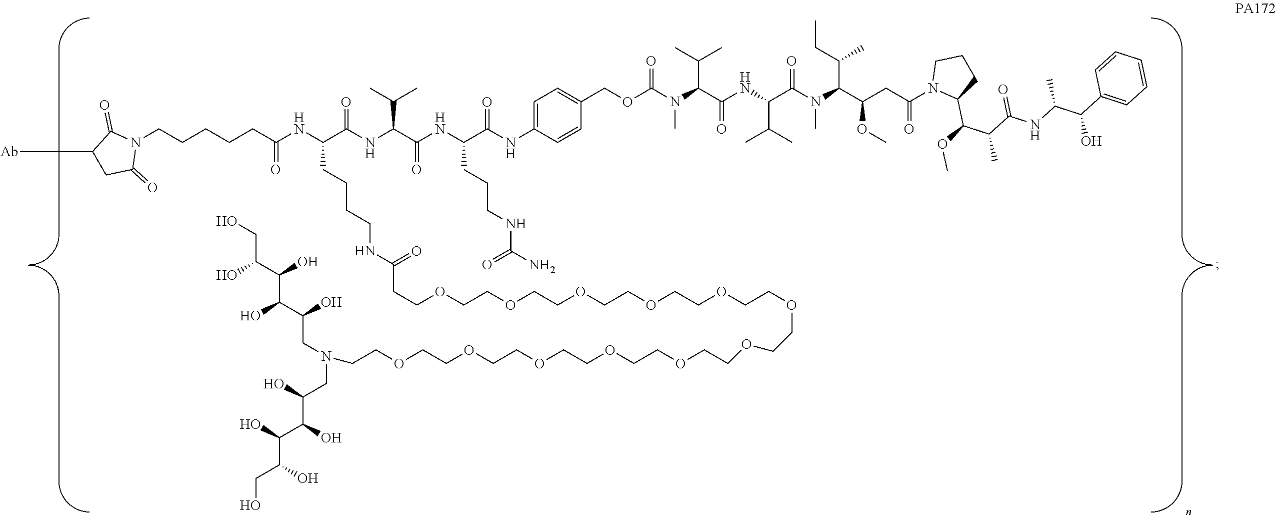

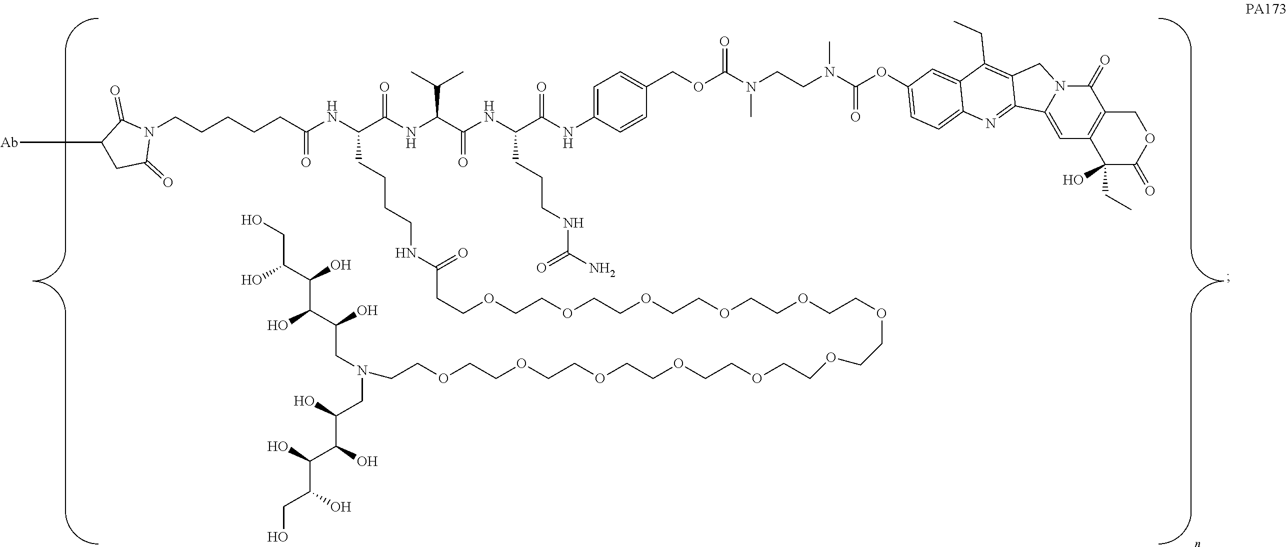

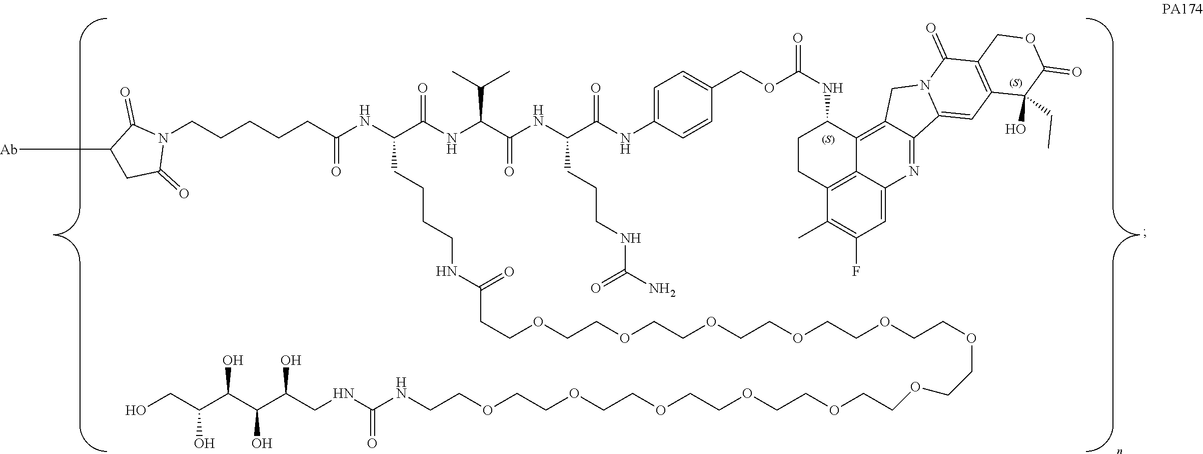

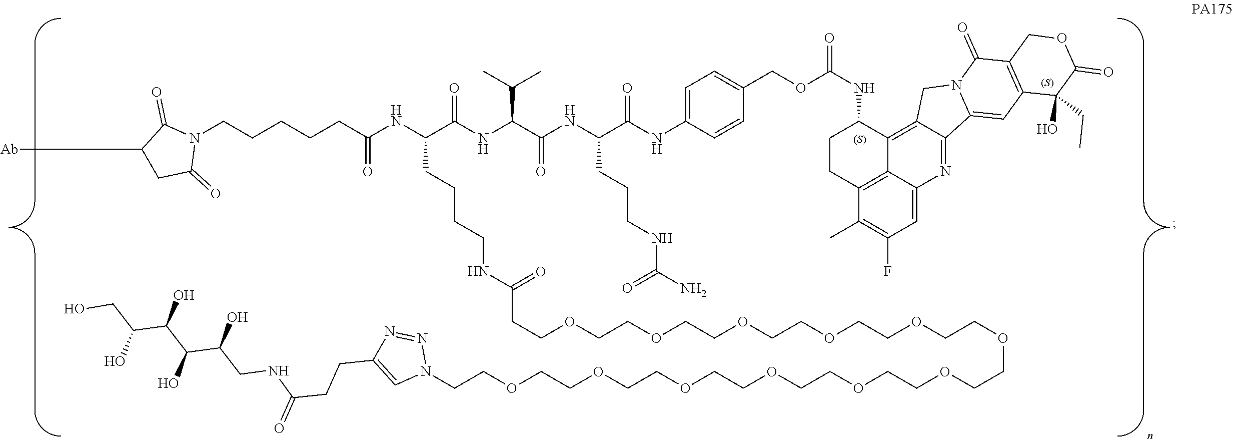

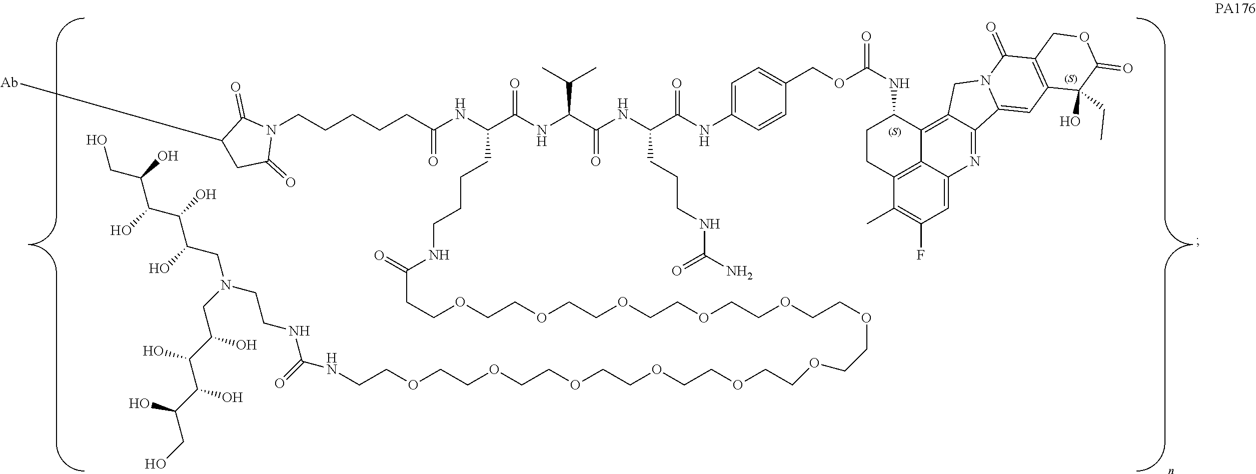

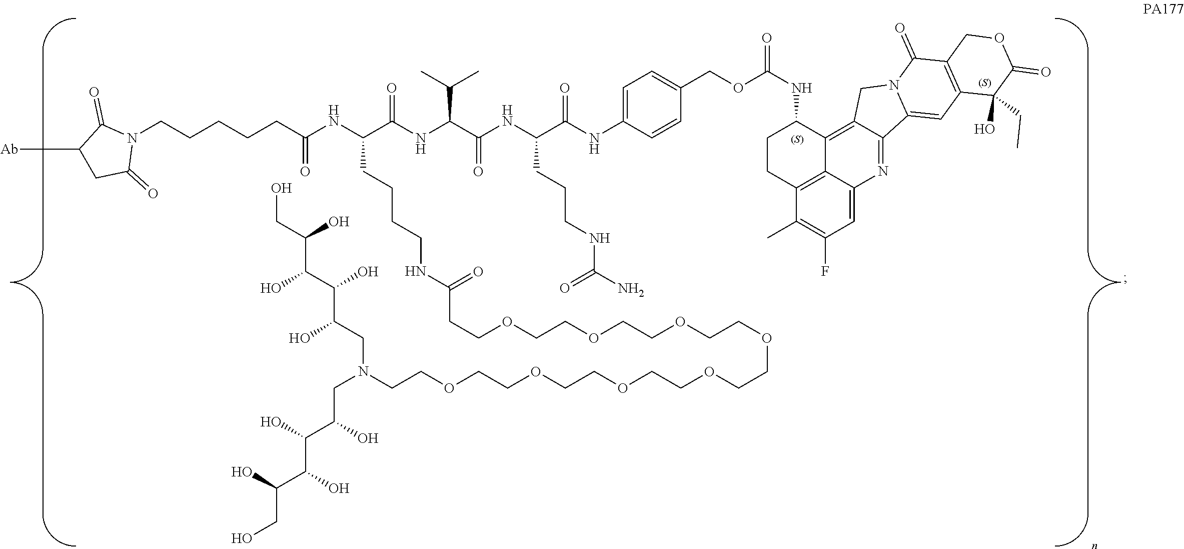

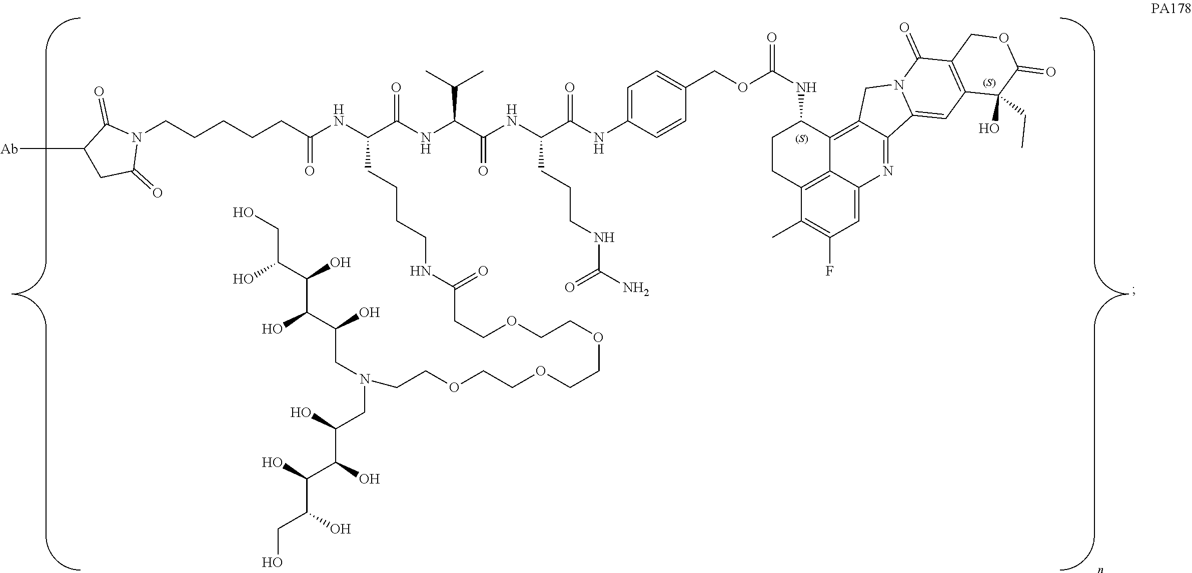

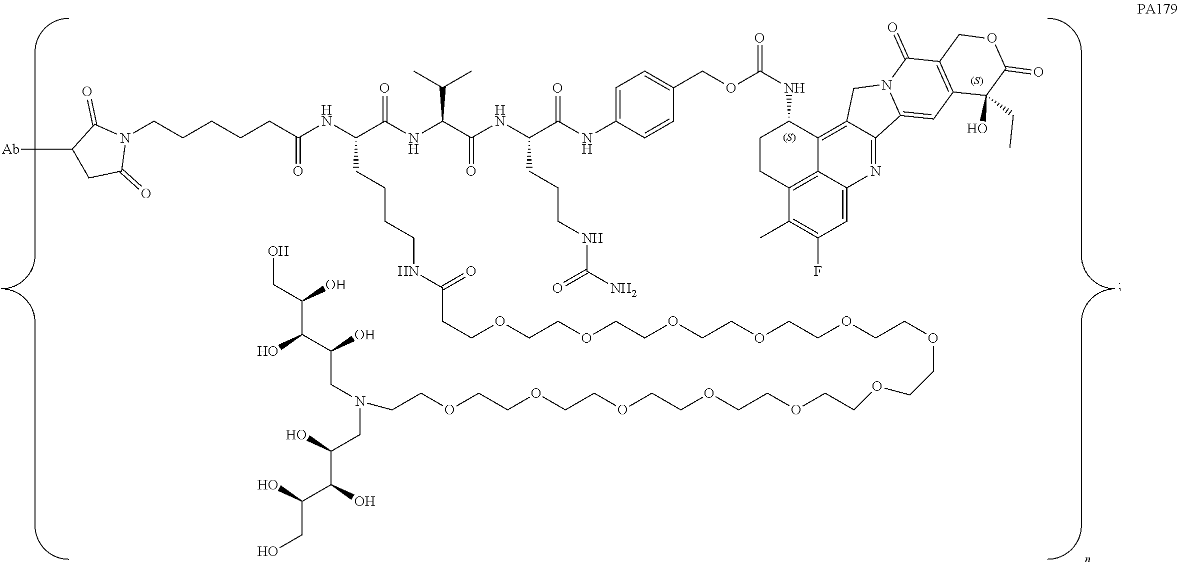

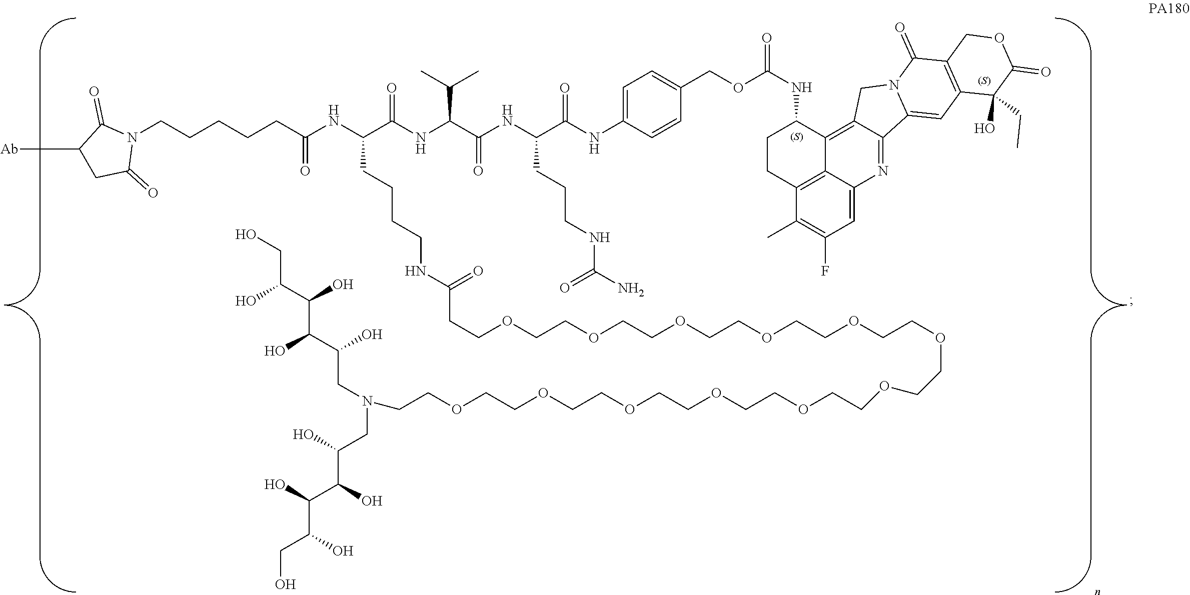

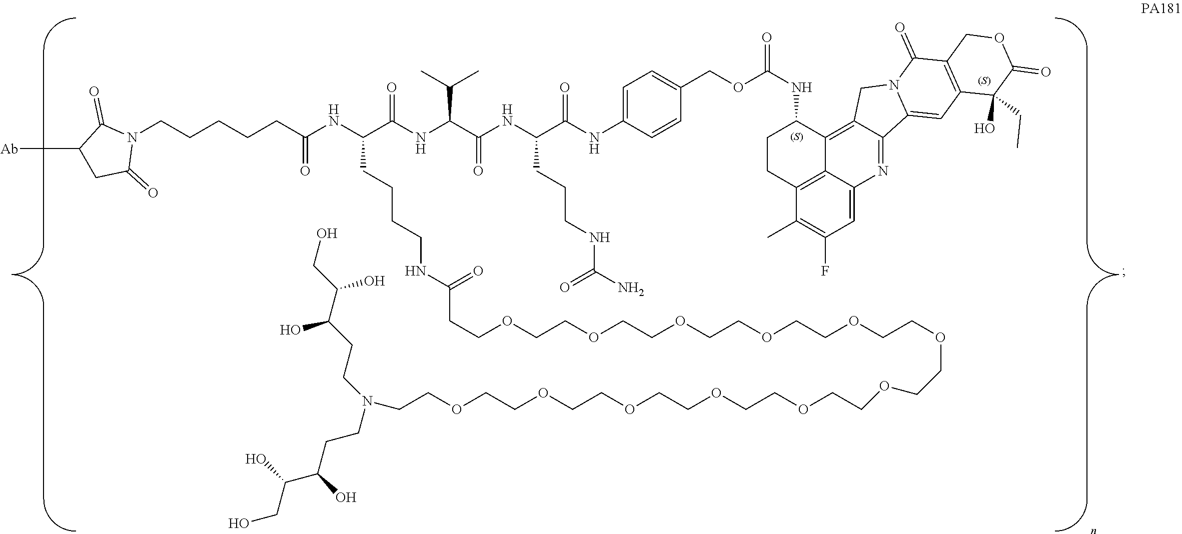

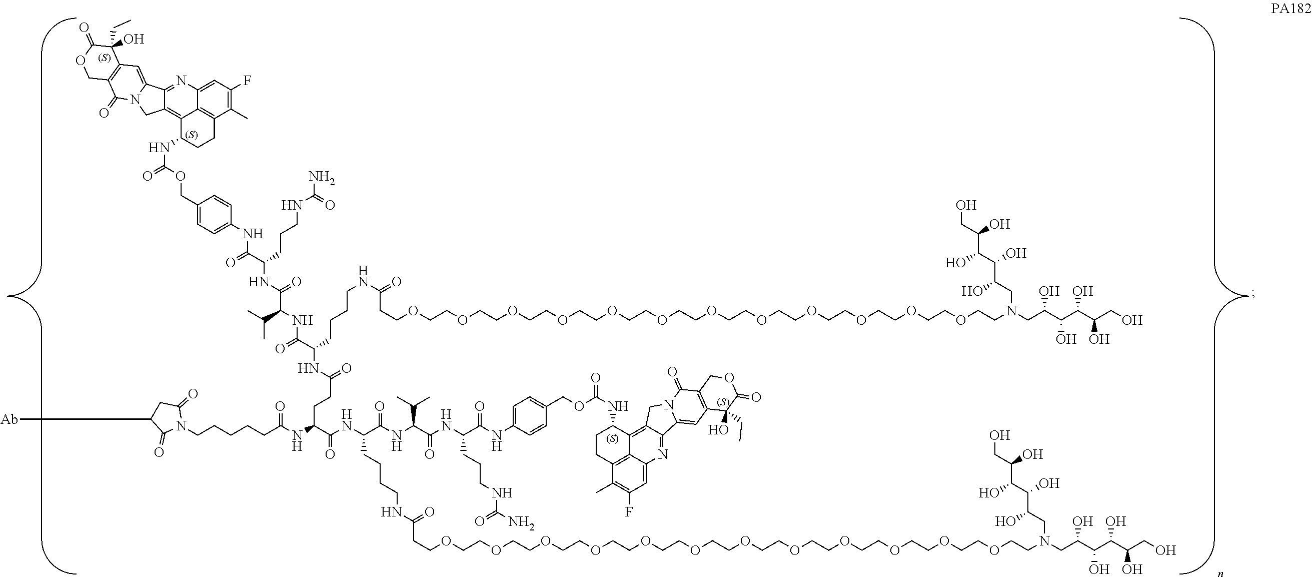

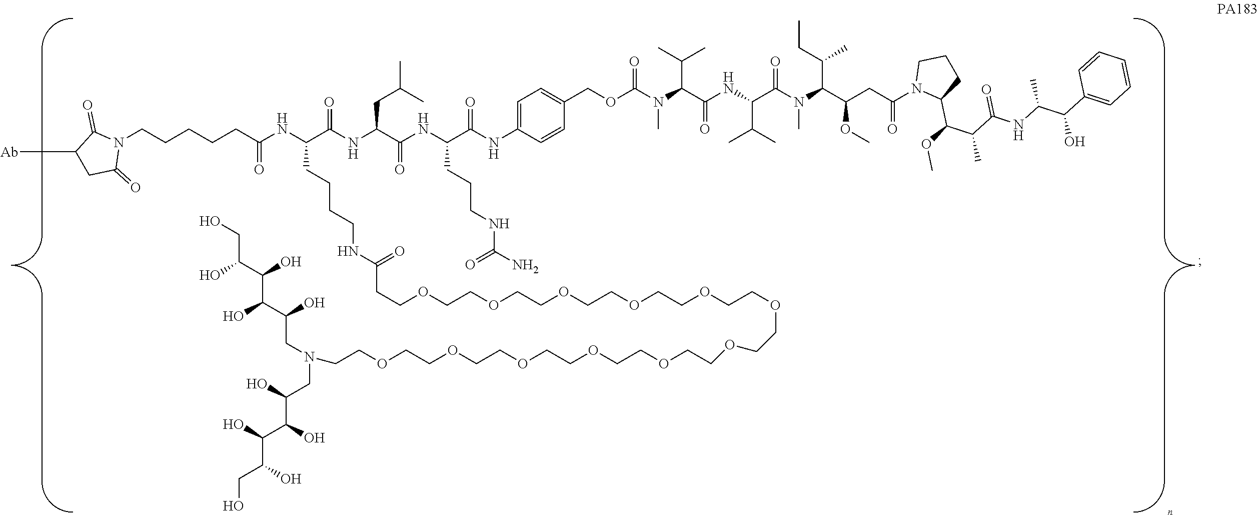

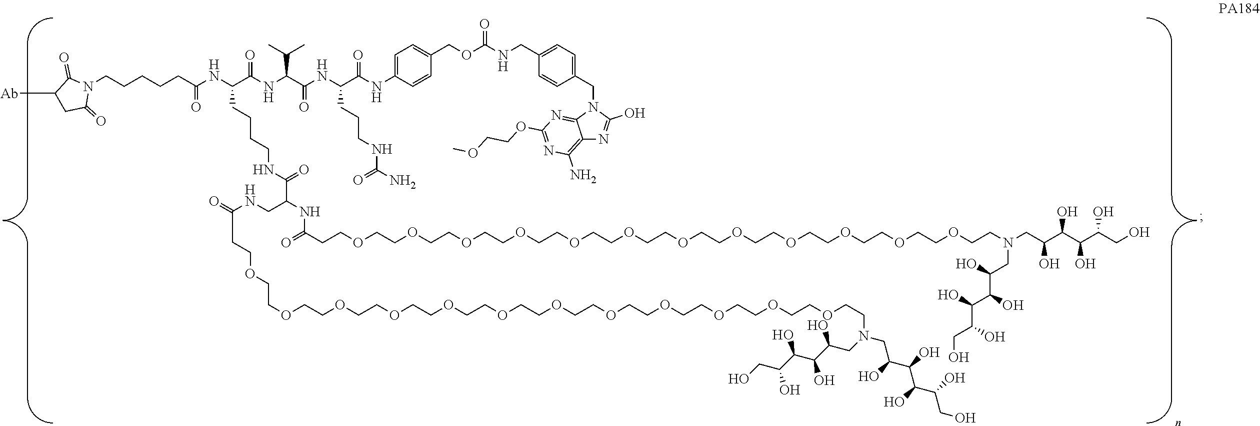

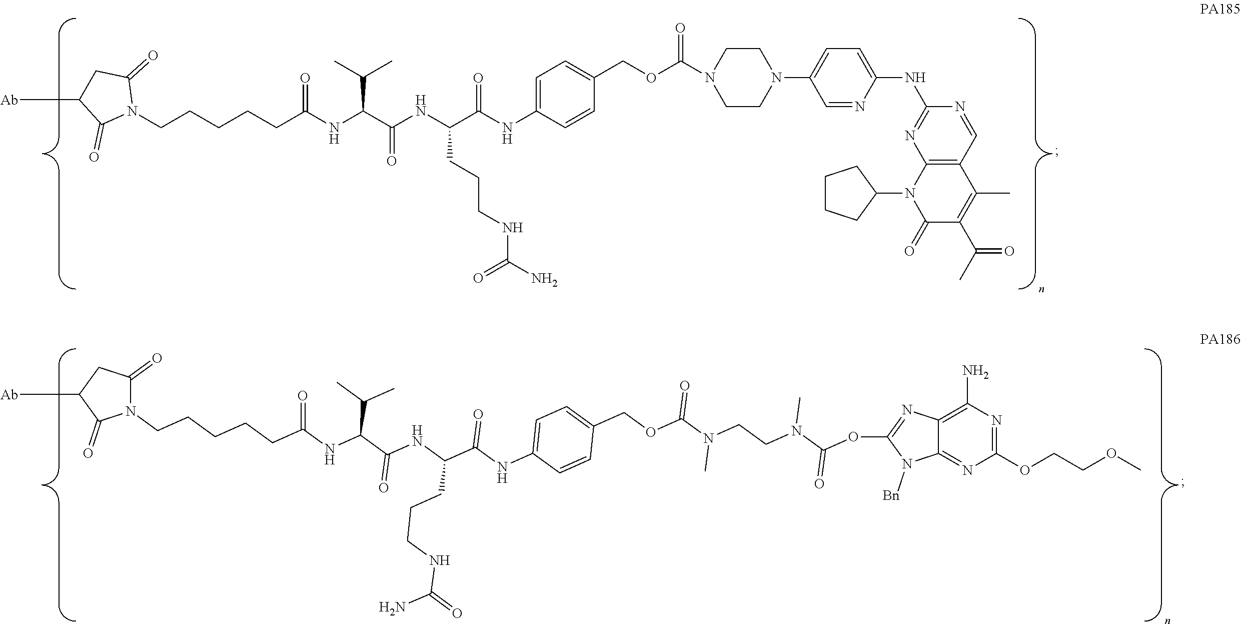

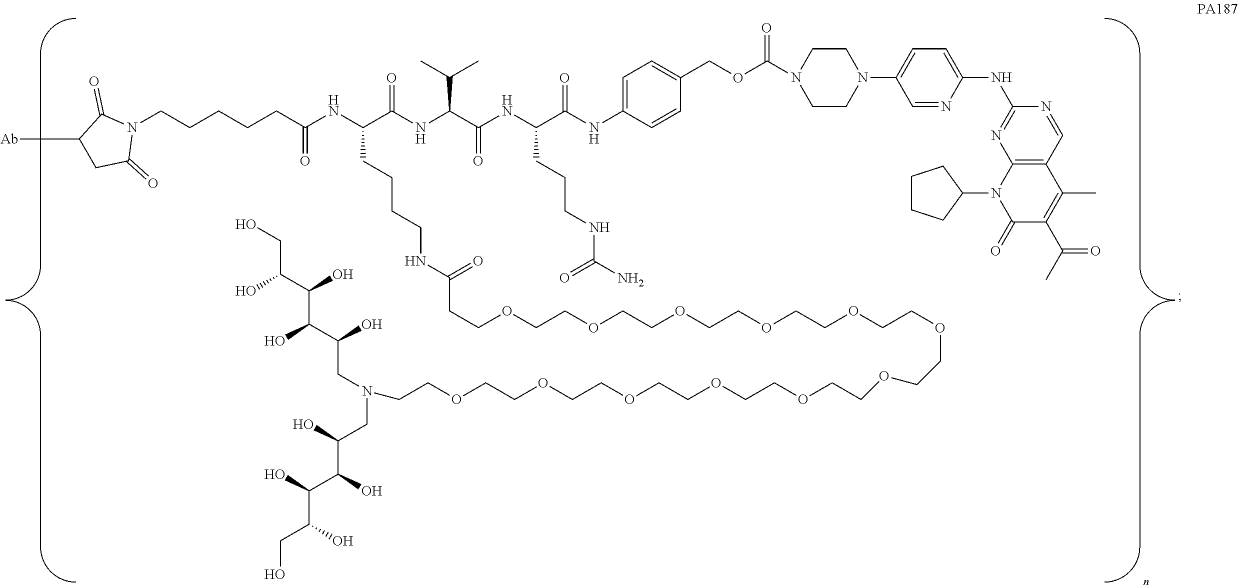

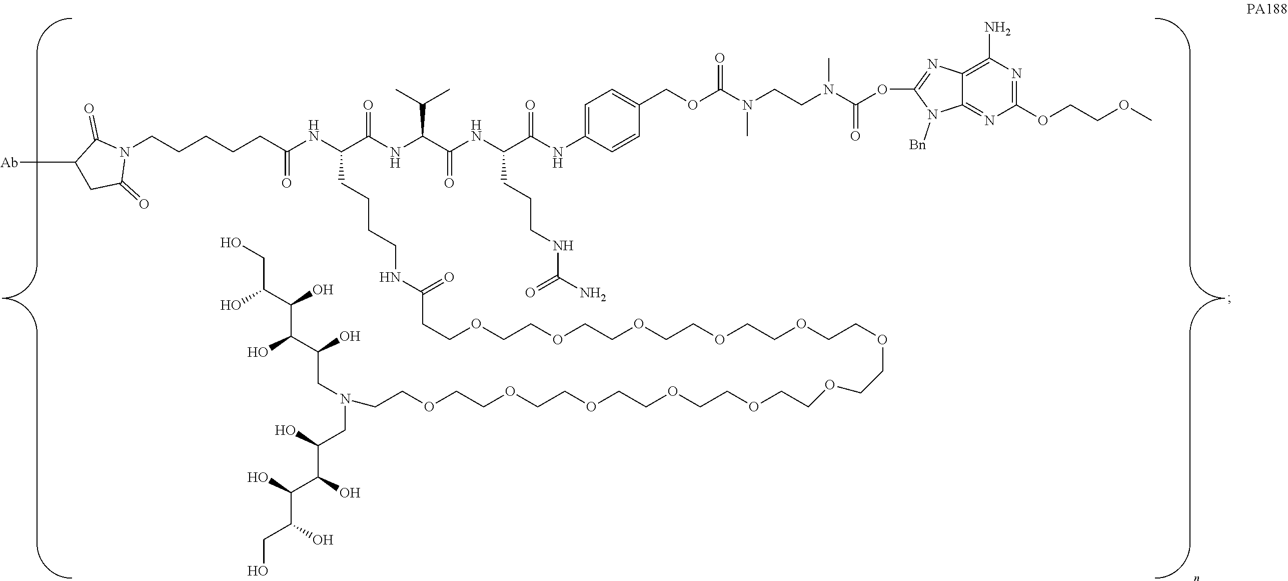

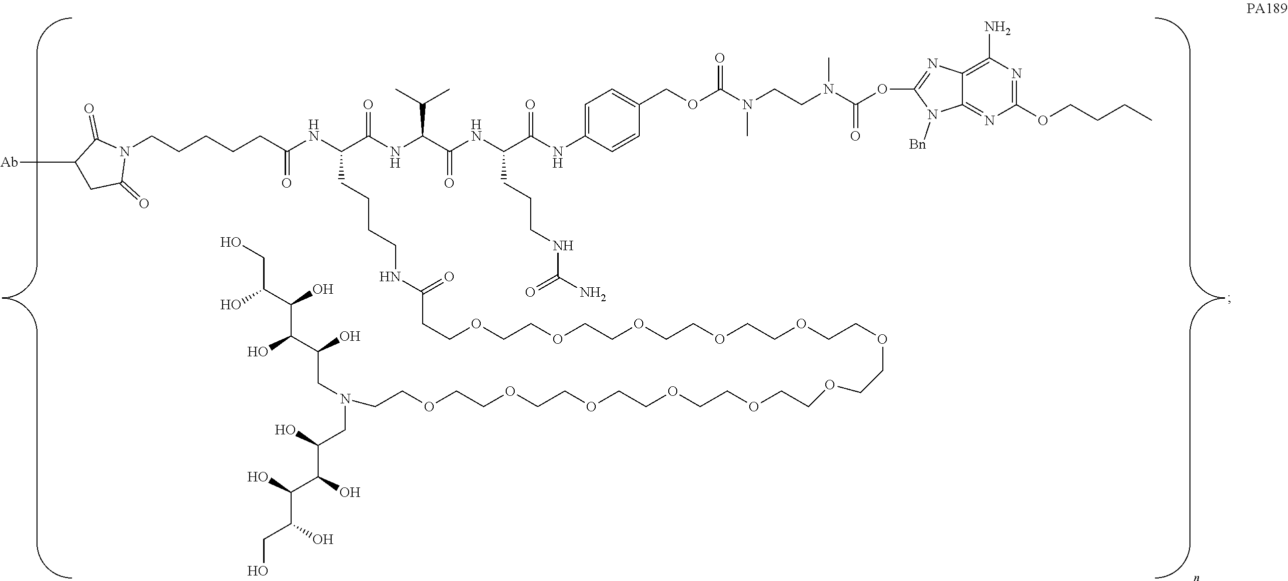

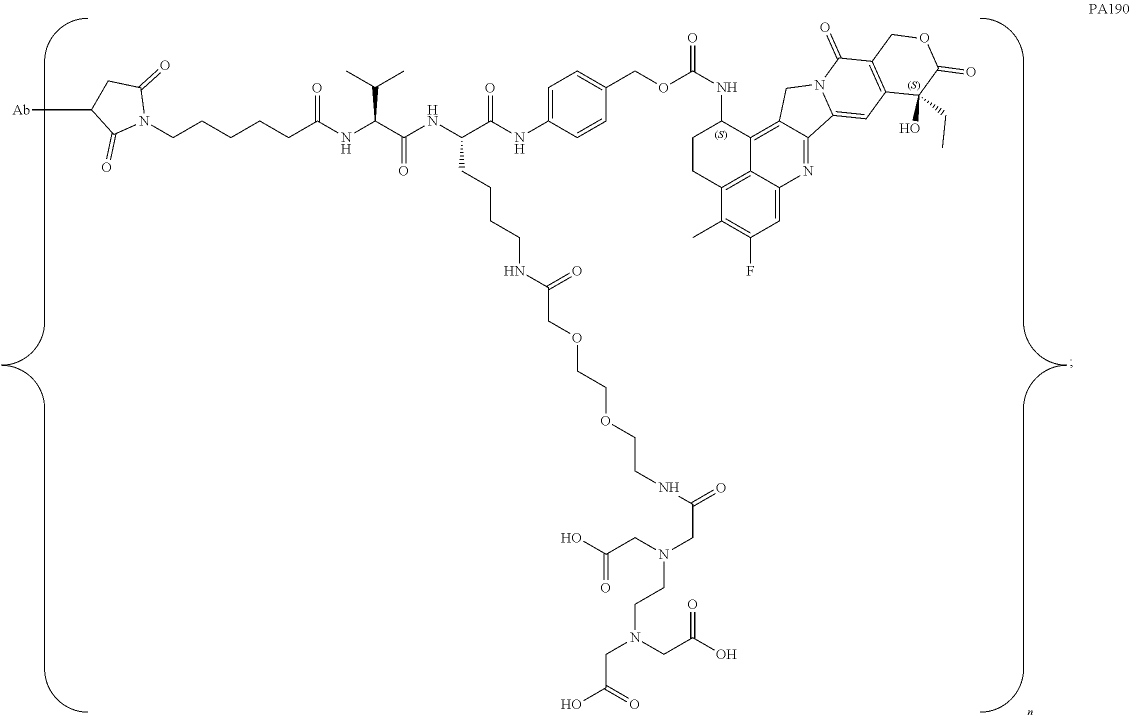

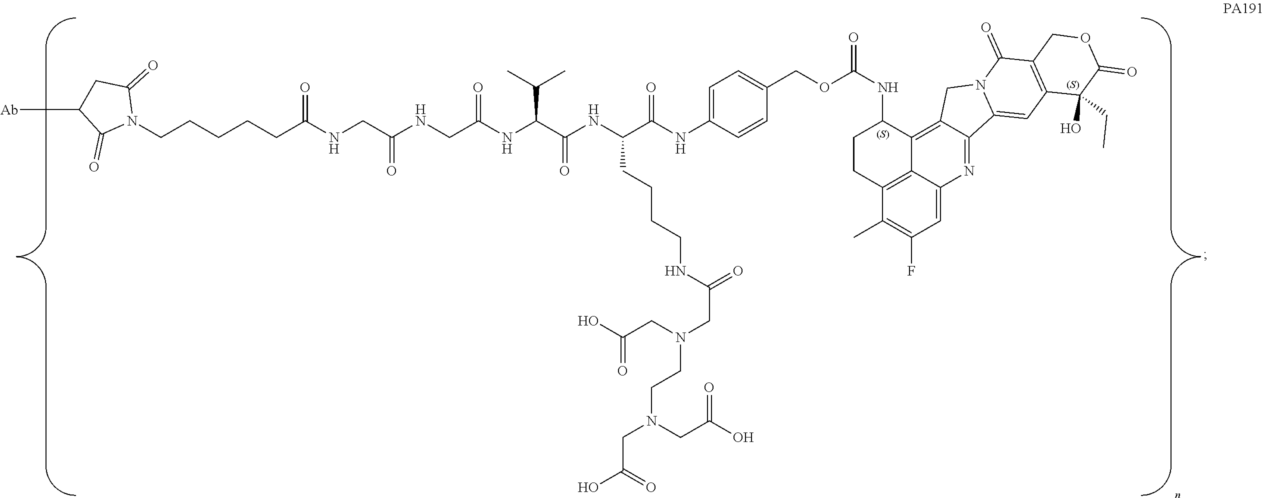

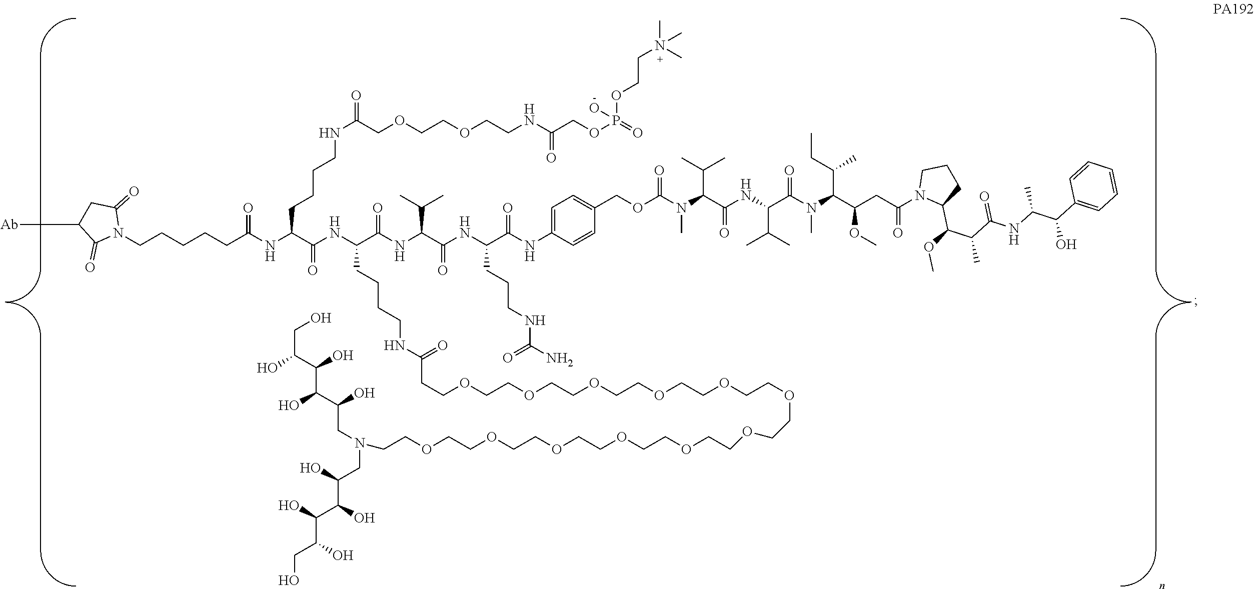

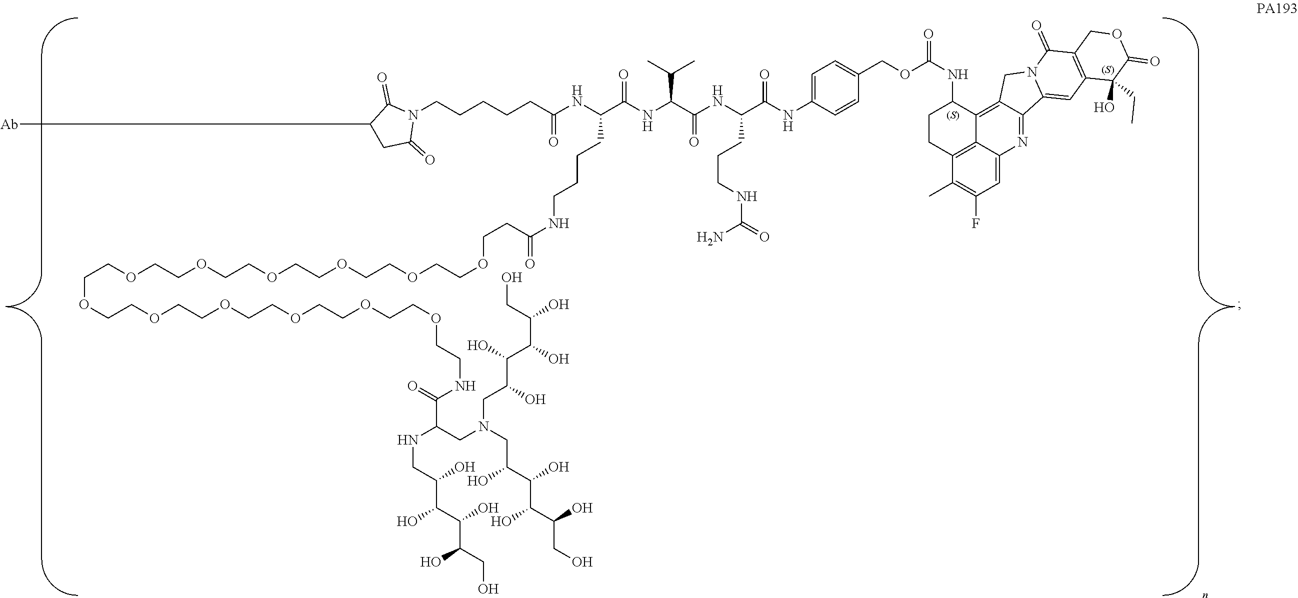

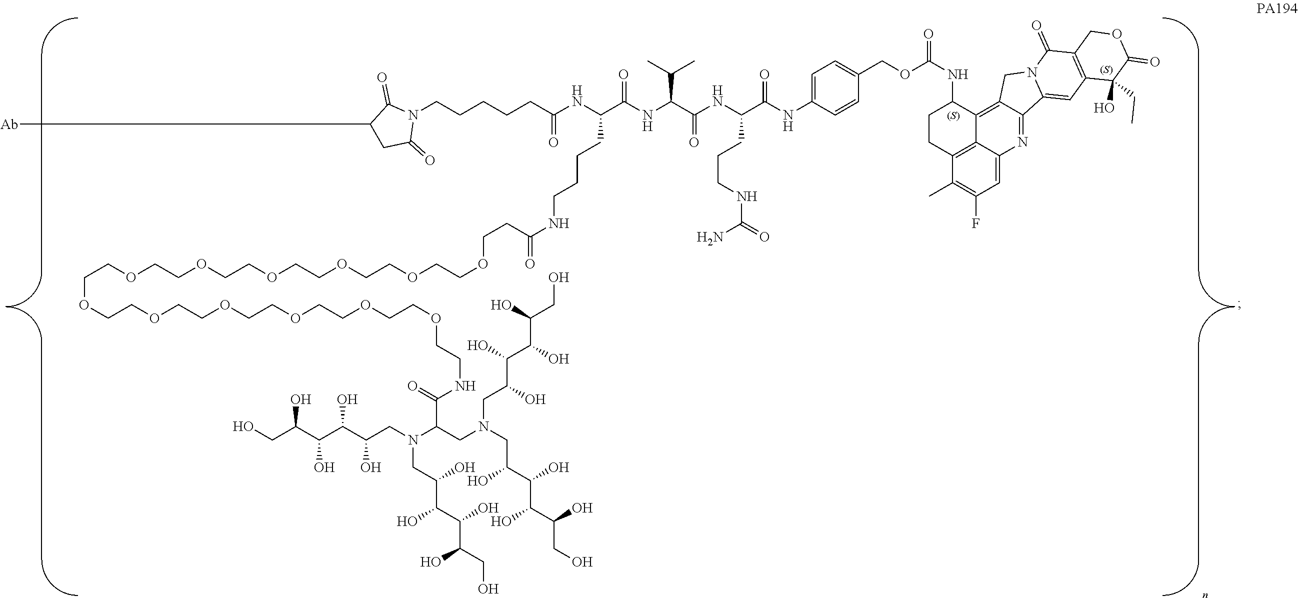

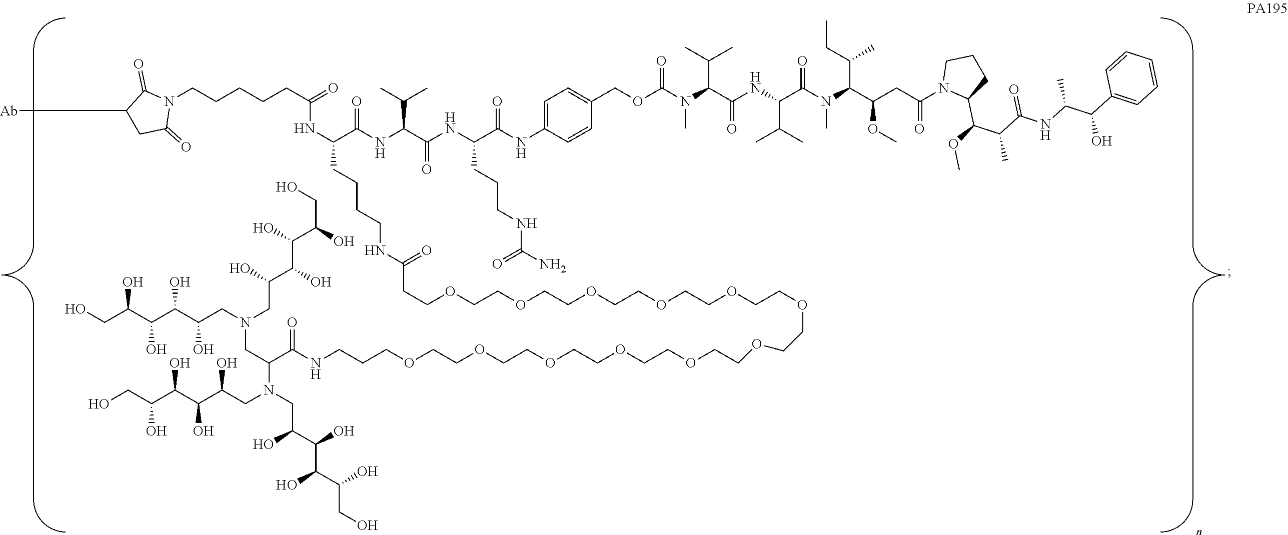

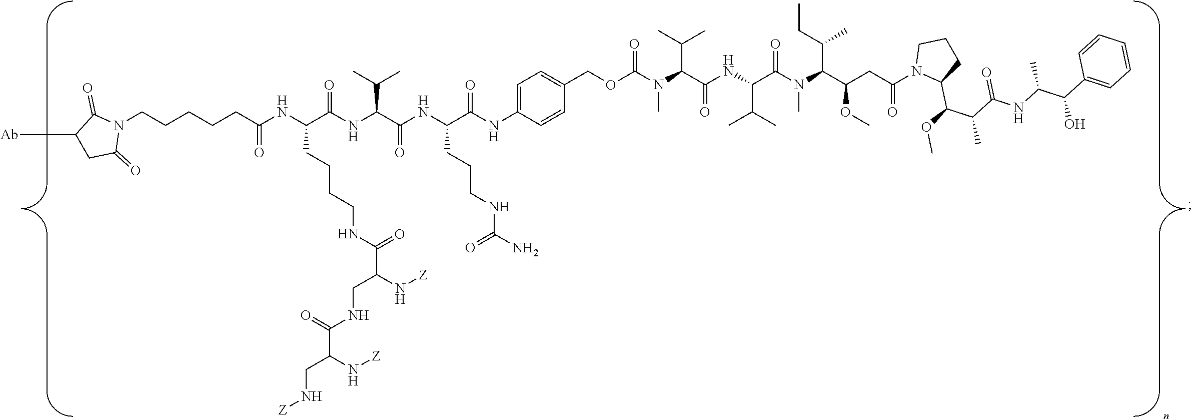

wherein each Z is attached at * and is individually selected from:

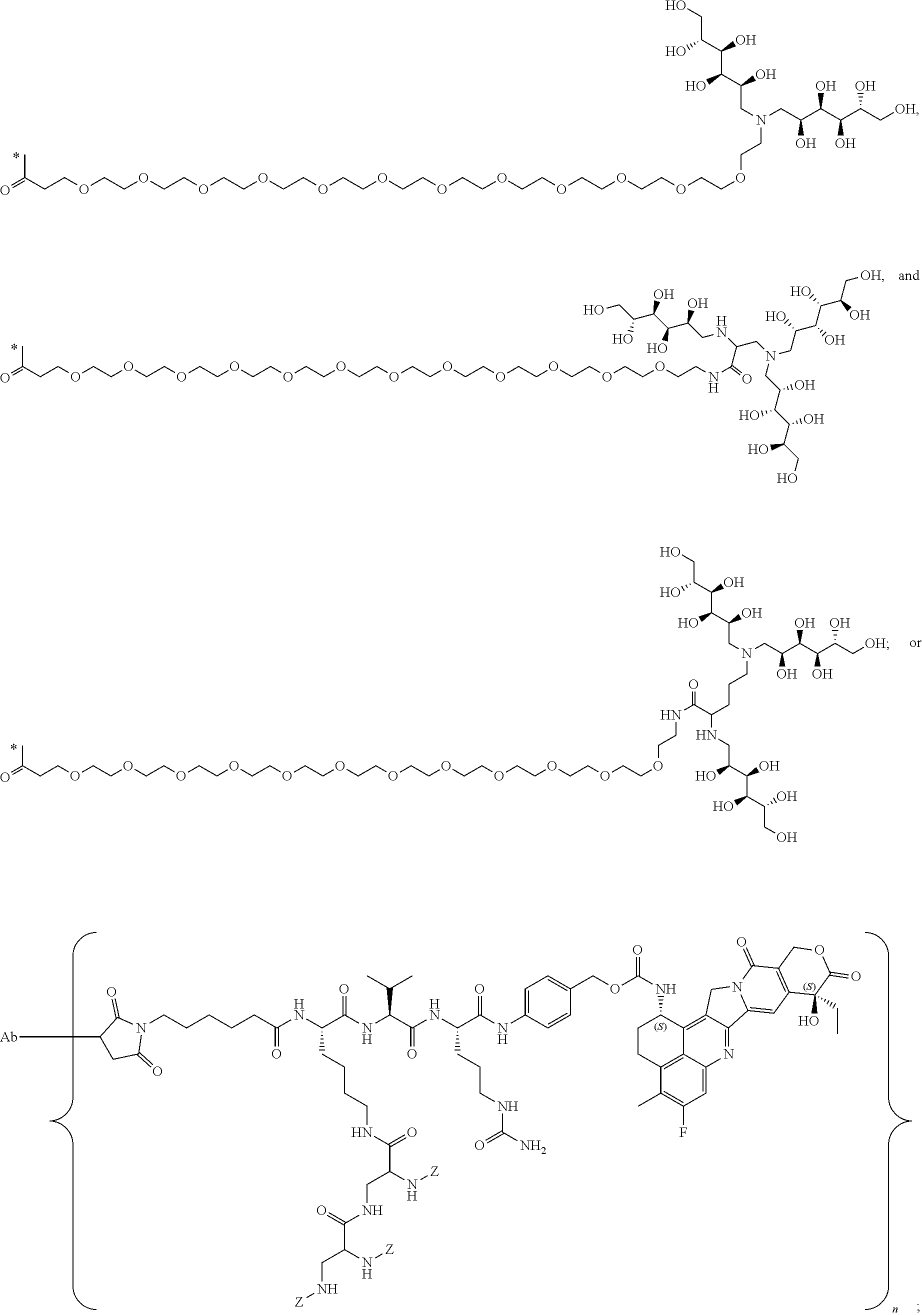

wherein each Z is attached at * and is individually selected from:

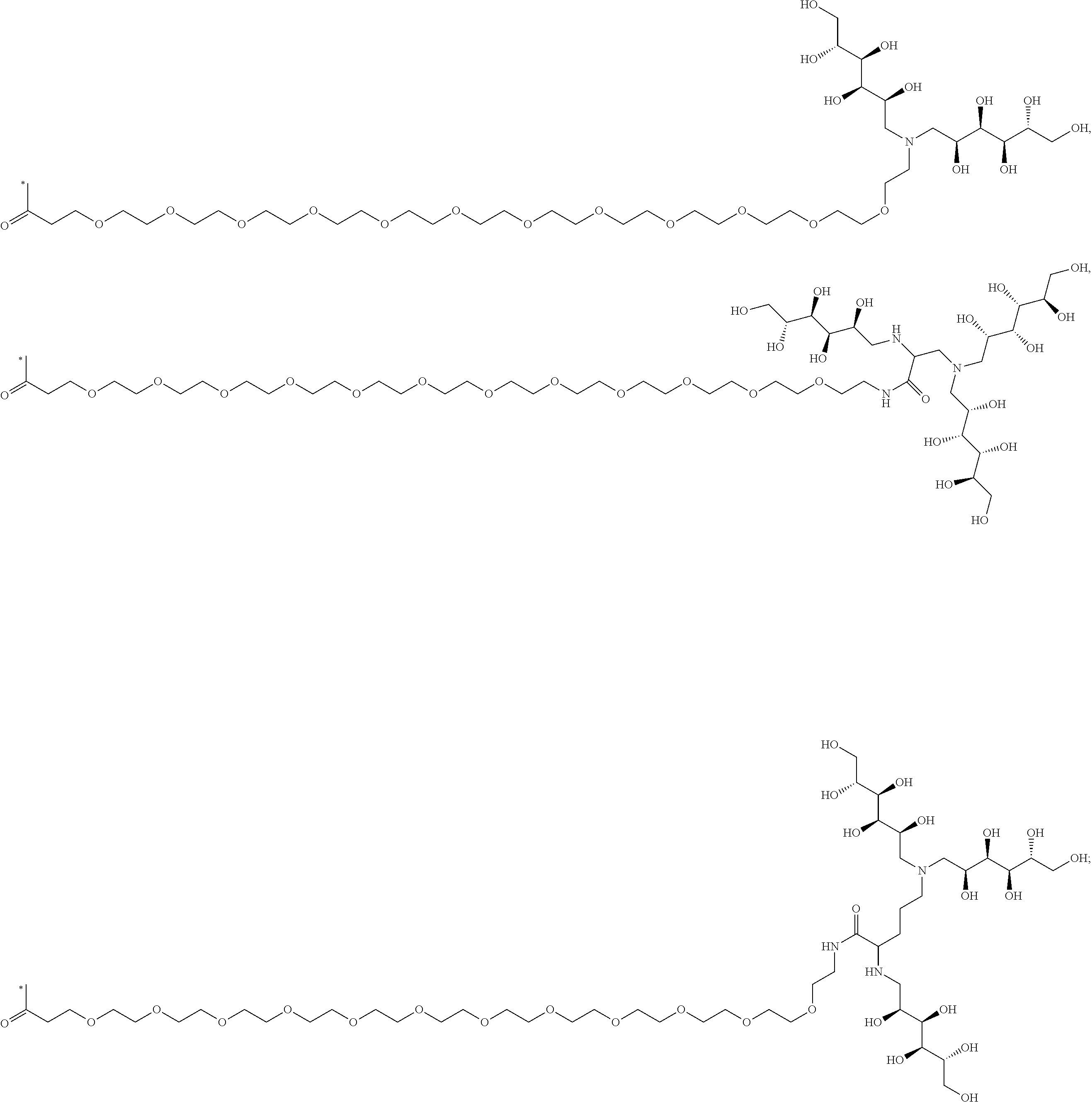 [SMILES: CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; CC(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO ; CC(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

or a stereoisomer thereof, wherein Ab represents the Binding Unit and n can be selected from p^{load}, such as for example. wherein p^{load} is from about 1 to about 8, about 2, about 4, about 6, about 8, about 10, about 12, about 14, about 16, about 3 to about 5, about 6 to about 8, or about 8 to about 16.

In some embodiments, provided is a conjugate as described above wherein the Binding unit is antibody 2E7 and the Drug-Linker is LD038. In a specific embodiment, the Binding unit is antibody 2E7 (VH SEQ ID NO: 7 and VL SEQ ID NO: 8).

In some embodiments, the conjugate has the following structure:

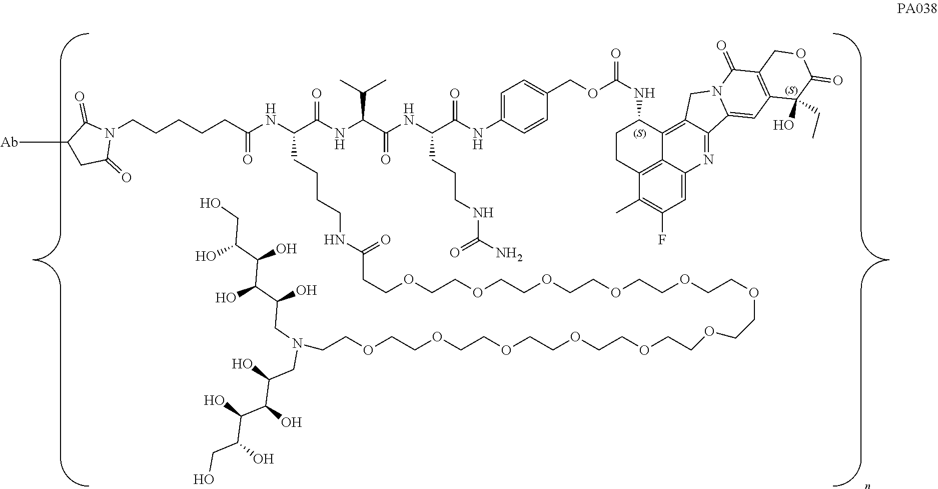

and wherein Ab is 2E7 and n can be selected from p^{load}, such as for example, wherein p^{load} is from about 1 to about 8, about 2, about 4, about 6, about 8, about 10, about 12, about 14, about 16, about 3 to about 5, about 6 to about 8, or about 8 to about 16.

In some embodiments, the conjugate has the following structure:

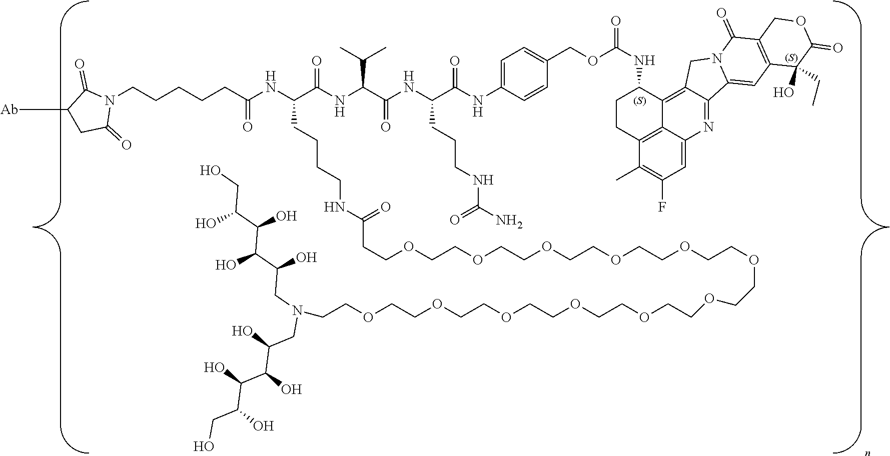

and wherein Ab is 2E7 and n can be selected from p^{load}, such as for example. wherein p^{load} is from about 1 to about 8, about 2, about 4, about 6, about 8, about 10, about 12, about 14, about 16, about 3 to about 5, about 6 to about 8, or about 8 to about 16.

### Exemplary Linker Drug Combinations

In some embodiments, provided is a conjugate wherein a Drug unit such as a tubulin disrupting agent, for example, an auristatin, is attached to the Linker by a C-terminal carboxyl group that forms an amide bond with the Linker Subunit L2. In some embodiments, provided is a conjugate wherein the Linker comprises at least one amino acid.

In some embodiments, provided is a conjugate wherein the Linker also comprises a Stretcher unit and/or an Amino Acid unit in addition to Linker Subunit L2.

In some embodiments, a Stretcher unit is capable of linking to a Binding unit to an Amino Acid unit or to a Linker Subunit L2 via a sulfhydryl group of the Binding unit. Sulfhydryl groups can be generated, for example, by reduction of the interchain disulfide bonds of a Binding unit. For example, a Stretcher unit can be linked to the Binding unit via the sulfur atoms generated from reduction of the interchain disulfide bonds of a Binding unit. In some embodiments, Stretcher units are linked to the Binding unit solely via the sulfur atoms generated from reduction of the interchain disulfide bonds of the Binding unit. In some embodiments, sulfhydryl groups can be generated by reaction of an amino group of a lysine moiety of a Binding unit with 2-iminothiolane (Traut's reagent) or other sulfhydryl generating reagents. In some embodiments, the Binding unit is a recombinant antibody and is engineered to contain one or more additional lysines. In some embodiments, a recombinant Binding unit is engineered to contain additional sulfhydryl groups, e.g., additional cysteines, such as engineered cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945, each of which is incorporated herein by reference in its entirety.

Representative Stretcher units are described within the square brackets of Formulas IIIa and IIIb of U.S. Pat. No. 9,211,319, and incorporated herein by reference.

In some embodiments, a CD70 conjugate comprises monomethyl auristatin E (MMAE) and a protease-cleavable Linker. In some embodiments, a CD70 conjugate comprises exatecan and a protease-cleavable Linker. It is contemplated that the protease cleavable Linker comprises a thiol-reactive spacer and a dipeptide. In various embodiments, the protease cleavable Linker includes a thiol-reactive maleimidocaproyl spacer, a valine-citrulline (val-cit) dipeptide, and a p-amino-benzyloxycarbonyl or PAB spacer.

The abbreviation “PAB” refers to the self-immolative spacer:

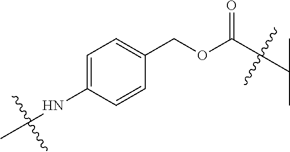

The abbreviation “MC” refers to the stretcher maleimidocaproyl:

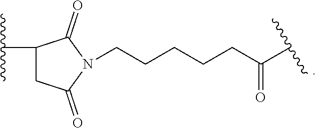 [SMILES: *C(=O)CCCCCN1C(=O)CC(*)C1=O]

In some embodiments, provided is a conjugate wherein the Drug unit is a camptothecin or a camptothecin (CPT) analog, such as irinotecan (also referred to as CPT-11), belotecan, topotecan, 10-hydroxy-CPT, exatecan, a diastereomer of exatecan, DXd, a diastereomer of DXdor SN-38. Representative structures are shown below.

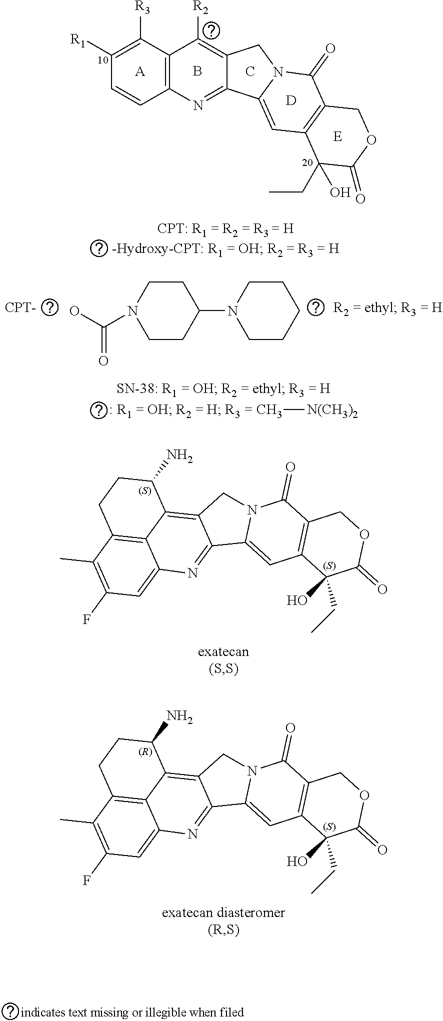

### Attachment of Drug-Linkers to Binding Units

Techniques for attaching Drug units to Binding units via Linkers are well-known in the art. See, e.g., Alley et al., Current Opinion in Chemical Biology 2010 14:1-9; Senter, Cancer J., 2008, 14(3):154-169. In some embodiments, provided is a conjugate wherein the Linker is first attached to a Drug unit (e.g., a cytotoxic agent(s)) and then the Drug-Linker is attached to the Binding unit. In some embodiments, provided is a conjugate wherein the Linker is first attached to a Binding unit, and then a Drug unit is attached to the Linker. In the following discussion, the term Drug-Linker is used to exemplify attachment of Linkers or Drug-Linkers to Binding units; the skilled artisan will appreciate that the selected attachment method can be determined according to Linker and the Drug unit (e.g., cytotoxic agent or other Drug unit). In some embodiments, provided is a conjugate wherein the Drug unit is attached to the Binding unit via a Linker in a manner that reduces the activity of the Drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a Binding unit with a bivalent Linker reagent to form a Binding unit-Linker intermediate via a covalent bond, followed by reaction with aDrug unit(s) (e.g., a cytotoxic agent); and (2) reaction of a nucleophilic group of a Drug unit(s) (e.g., a cytotoxic agent) with a bivalent Linker reagent, to form Drug-Linker, via a covalent bond, followed by reaction with a nucleophilic group of the Binding unit. Exemplary methods for preparing conjugates via the latter route are described in U.S. Pat. No. 7,498,298, which is expressly incorporated herein by reference.

Nucleophilic groups on Binding units include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain Binding units have reducible interchain disulfides, i.e., cysteine bridges. Binding units may be made reactive for conjugation with Linker reagents by treatment with a reducing agent such as DTT (dithiothreitol) or tricarbonylethylphosphine (TCEP), such that the Binding unit is fully or partially reduced. Each cysteine bridge will thus form, theoretically, two reactive thiol nucleophiles. Additional nucleophilic groups can be introduced into Binding units through modification of lysine residues, e.g., by reacting lysine residues with 2-iminothiolane (Traut's reagent), resulting in conversion of an amine into a thiol. Reactive thiol groups may also be introduced into Binding units by introducing one, two, three, four, or more cysteine residues (e.g., by preparing Binding units comprising one or more non-native cysteine amino acid residues).

Conjugates may also be produced by reaction between an electrophilic group on a Binding unit, such as an aldehyde or ketone carbonyl group, with a nucleophilic group on a Linker reagent or Drug unit. Useful nucleophilic groups on a Linker reagent include, but are not limited to, hydrazide, oxime, amino, hydrazine, thiosemicarbazone, hydrazine carboxylate, and aryl hydrazide. In an embodiment, a Binding unit is modified to introduce electrophilic moieties that are capable of reacting with nucleophilic substituents on the Linker reagent or Drug unit. In another embodiment, the sugars of glycosylated Binding units may be oxidized, e.g. with periodate oxidizing reagents, to form aldehyde or ketone groups which may react with the amine group of Linker reagents or Drug unit moieties. The resulting imine Schiff base groups may form a stable linkage, or may be reduced, e.g., by borohydride reagents to form stable amine linkages. In one embodiment, reaction of the carbohydrate portion of a glycosylated Binding unit with either galactose oxidase or sodium meta-periodate may yield carbonyl (aldehyde and ketone) groups in the Binding unit that can react with appropriate groups on the Drug unit (see, e.g., Hermanson, Bioconjugate Techniques). In another embodiment, Binding units containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a cytotoxic agent or Linker.

Exemplary nucleophilic groups on a Drug unit, such as a cytotoxic agent, include, but are not limited to: amine, thiol, hydroxyl, hydrazide, oxime, hydrazine, thiosemicarbazone, hydrazine carboxylate, and aryl hydrazide groups capable of reacting to form covalent bonds with electrophilic groups on Linker moieties and Linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, provided is a conjugate wherein the Drug-Linker is attached to an interchain cysteine residue(s) of a Binding unit). See, e.g., WO2004/010957 and WO2005/081711. In such embodiments, the Linker typically comprises a maleimide group for attachment to the cysteine residues of an interchain disulfide. In some embodiments, provided is a conjugate wherein the Linker or Drug-Linker is attached to a cysteine residue(s) of Binding unit as described in U.S. Pat. Nos. 7,585,491 or 8,080,250.

In some embodiments, provided is a conjugate wherein the Linker or Drug-Linker is attached to a lysine or cysteine residue(s) of a Binding unit as described in WO2005/037992 or WO2010/141566.

In some embodiments, provided is a conjugate wherein engineered cysteine residues, polyhistidine sequences, glycoengineering tags, or transglutaminase recognition sequences can be used for site-specific attachment of Linkers or Drug-linkers to Binding units.

In some embodiments, provided is a conjugate wherein the Drug-Linker(s) is attached to an engineered cysteine residue at an Fc residue other than an interchain disulfide. In some embodiments, provided is a conjugate wherein the Drug-Linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, provided is a conjugate wherein the Linker or Drug-linker(s) is attached to one or more introduced cysteine residues of a Binding unit as described in WO2006/034488, WO2011/156328 and/or WO2016040856.

In some embodiments, provided is a conjugate wherein an exemplary substitution for site specific conjugation using bacterial transglutaminase is N297S or N297Q of the Fc region. In some embodiments, provided is a conjugate wherein the Linker or Drug-linker(s) is attached to the glycan or modified glycan of a Binding unit). See, e.g., WO2017/147542, WO2020/123425, WO2020/245229, WO2014/072482; WO2014//065661, WO2015/057066 and WO2016/022027; the disclosure of which are incorporated by reference herein.

### Drug Loading

The CD70 ADCs can comprise one or more Drug units per Binding unit. The number of Drug units per Binding unit is referred to as drug loading. The drug loading of a CD70 ADC is represented by p^{load}, the average number of Drug units (drug molecules (e.g., cytotoxic agents)) per Binding unit (e.g., an antibody or antigen binding portion) in a CD70 ADC. For example, if p^{load} is about 4, the average drug loading taking into account all of the Binding units (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of Drug units per Binding unit (e.g., antibody or antigen binding portion) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Pharmaceutical Formulations

Other aspects relate to compositions comprising CD70 conjugates as described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A CD70 conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a CD70 conjugate). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., a CD70 conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a CD70 conjugate as described herein can be a lyophilisate.

In some embodiments, a syringe comprising a therapeutically effective amount of a CD70 conjugate, or a pharmaceutical composition thereof, described herein is provided.

### Treatment of Cancer

In some embodiments, the CD70 conjugates as described herein can be used in a method(s) comprising administering a CD70 conjugate as described herein to a subject in need thereof, such as a subject having cancer.

In some embodiments, provided are methods of treating cancer comprising administering a CD70 conjugate comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:3 and SEQ ID NO:4, respectively. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:5 and SEQ ID NO:6, respectively. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:7 and SEQ ID NO:8, respectively. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:9 and SEQ ID NO:10, respectively. In some embodiments, provided are methods of treating cancer comprising administering a CD70 conjugate comprising a heavy chain variable region (VH) and a light chain variable region (VL), the VH and VL regions having amino acid sequences set forth in and SEQ ID NO:11 and SEQ ID NO:12; respectively.

In some embodiments, provided are methods of treating cancer comprising administering a CD70 conjugate comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, provided are methods of treating cancer comprising administering a CD70 conjugate comprising a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating cancer comprising administering a CD70 conjugate comprising a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are method of treating cancer comprising administering a CD70 conjugate comprising a heavy chain variable region having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 3, 5, 7, 9, or 11. In some embodiments, provided are method of treating cancer comprising administering a CD70 conjugate comprising a VL region having the amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any of SEQ ID NOs: 4, 6, 8, 10, or 12.

In some embodiments, provided are methods of treating cancer comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided are methods of treating cancer comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating cancer comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating cancer comprising administering a CD70 conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiment of treating cancer, each VH and VL region comprises a human framework region.

In some embodiments, provided are methods of treating cancer comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided are methods of treating cancer comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the subject is in need of treatment for a cancer and/or a malignancy. In some embodiments, the subject is in need of treatment for a CD70+ cancer or a CD70+ malignancy, such as for example, hepatocellular cancer, colorectal cancer, pancreatic cancer, ovarian cancer, indolent Non-Hodgkin's Lymphoma (indolent NHLs) (e.g., follicular NHLs, small lymphocytic lymphomas, lymphoplasmacytic NHLs, or marginal zone NHLs), Non-Hodgkin's Lymphoma (non-indolent), cancers of the B-cell lineage, including, e.g., Burkitt's lymphoma and chronic lymphocytic leukemia, multiple myeloma, renal cell cancers, nasopharyngeal cancers, thymic cancers, squamous cell carcinomas, head and neck cancers, and gliomas. In some embodiments, the method is for treating a subject having a CD70+ cancer or malignancy. In some embodiments, the method is for treating hepatocellular cancer in a subject. In some embodiments, the method is for treating colorectal cancer in a subject. In some embodiments, the method is for treating pancreatic cancer in a subject. In some embodiments, the method is for treating ovarian cancer in a subject. In some embodiments, the method is for treating an indolent Non-Hodgkin's Lymphoma (indolent NHLs), such as for example a follicular NHL, a small lymphocytic lymphoma, a lymphoplasmacytic NHL, or a marginal zone NHL in a subject. In some embodiments, the method is for treating Non-Hodgkin's Lymphoma, for example, diffuse large B cell lymphoma (DLBCL), in a subject. In some embodiments, the method is for treating cancers of the B-cell lineage, such as, for example, Burkitt's lymphoma or chronic lymphocytic leukemia, in a subject. In some embodiments, the method is for treating multiple myeloma in a subject. In some embodiments, the method is for treating renal cell cancer in a subject. In some embodiments, the method is for treating nasopharyngeal carcinoma in a subject. In some embodiments, the method is for treating thymic cancer in a subject. In some embodiments, the method is for treating a glioma in a subject. In some embodiments, the method is for treating a hematologic malignancy in a subject. In some embodiments, the method is for treating a squamous cell carcinoma in a subject. In some embodiments, the method is for treating renal cell carcinoma, for example, clear cell renal cell carcinoma (ccRCC), in a subject. In some embodiments, the method is for treating head and neck cancers, for example, head and neck squamous cell carcinoma (HNSCC), in a subject.

The methods described herein include administering a therapeutically effective amount of a CD70 conjugate to a subject having a CD70+ cancer or malignancy. As used herein, the phrases “therapeutically effective amount”, “effective amount” or “effective dose” refer to an amount of the CD70 conjugate as described herein that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), clear cell renal cancer (ccRCC), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, diffuse large B cell lymphoma (DLBCL), melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the cancer is a solid tumor. In some embodiments, the cancer is selected from a solid tumor, including but not limited to, hepatocellular cancer, colorectal cancer, renal cell carcer, pancreatic cancer, ovarian cancer, nasopharyngeal carcinoma, thymic cancer and gliomas. In some embodiments, the cancer is selected from a hematologic cancer, also referred to as a hematologic malignancy. In some embodiments, the cancer is selected from a hematologic cancer, such as indolent Non-Hodgkin's Lymphoma (indolent NHLs) (e.g., follicular NHLs, small lymphocytic lymphomas, lyiphoplasmacytic NHLs, or marginal zone NHLs), Non-Hodgkin's Lyiphoma (non-indolent), NS cancers of the B-cell lineage, including, e.g., Burkitt's lymphoma and chronic lymphocytic leukemia. In some embodiments, the cancer is Non-Hodgkin lymphoma. In some embodiments, the cancer is diffuse large B cell lymphoma (DLBCL). In some embodiments, the cancer is renal cell carcinoma. In some embodiments, the cancer is clear cell renal cell carcinoma (ccRCC). In some embodiments, the cancer is head and neck cancer. In some embodiments, the cancer is head and neck squamous cell carcinoma (HNSCC). In some embodiments, the cancer or malignancy is CD70-positive (CD70+). As used herein, the terms “CD70-positive” or “CD70+” are used to describe a cancer cell, a cluster of cancer cells, a tumor mass, or a metastatic cell that express CD70 on the cell surface (membrane-bound CD70). Some non-limiting examples of CD70-positive cancers include, for example, hepatocellular cancer, colorectal cancer, pancreatic cancer, ovarian cancer, indolent Non-Hodgkin's Lymphoma (indolent NHLs) (e.g., follicular NHLs, small lymphocytic lymphomas, lymphoplasmacytic NHLs, or marginal zone NHLs), Non-Hodgkin's Lymphoma, cancers of the B-cell lineage, including, e.g., Burkitt's lymphoma and chronic lymphocytic leukemia, multiple myeloma, renal cell cancers, nasopharyngeal cancers, thymic cancers, head and neck cancers, squamous cell carcinomas, and gliomas. In some embodiments, the CD70-positive cancer is Non-Hodgkin lymphoma. In some embodiments, the CD70-positive cancer is diffuse large B cell lymphoma (DLBCL). In some embodiments, the CD70-positive cancer is renal cell carcinoma. In some embodiments, the CD70-positive cancer is clear cell renal cell carcinoma (ccRCC). In some embodiments, the CD70-positive cancer is head and neck cancer. In some embodiments, the CD70-positive cancer is head and neck squamous cell carcinoma (HNSCC).

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a CD70+ cancer and in need of treatment, but need not have already undergone treatment for the CD70+ cancer. In some embodiments, a subject can also be one who has not been previously diagnosed as having a CD70+ cancer in need of treatment. In some embodiments, a subject can be one who exhibits one or more risk factors for a condition or one or more complications related to a CD70+ cancer or a subject who does not exhibit risk factors. A “subject in need” of treatment for a CD70+ cancer particular can be a subject having that condition or diagnosed as having that condition. In other embodiments, a subject “at risk of developing” a condition refers to a subject diagnosed as being at risk for developing the condition or at risk for having the condition again (e.g., a CD70+ cancer).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in CD70+ cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a CD70 conjugate as described herein into a subject by a method or route which results in binding of the CD70 conjugate to CD70+ cancer cells or malignant cells. Similarly, a pharmaceutical composition comprising a CD70 conjugate as described herein can be administered by any appropriate route which results in an effective treatment in the subject.

The dosage ranges for a CD70 conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a CD70 conjugate can be administered in a unit dose. The term “unit dose” when used in reference to a pharmaceutical composition refers to physically discrete units suitable as unitary dosage for the subject, each unit containing a predetermined quantity of active material (e.g., a CD70 conjugate), calculated to produce the desired therapeutic effect in association with the required physiologically acceptable diluent, i.e., carrier, or vehicle.

In some embodiments, a CD70 conjugate, or a pharmaceutical composition thereof, is administered with an immunotherapy. As used herein, “immunotherapy” refers to therapeutic strategies designed to induce or augment the subject's own immune system to fight the cancer or malignancy. Examples of an immunotherapy include, but are not limited to, antibodies such as check point inhibitors.

In some embodiments, the immunotherapy involves administration of a checkpoint inhibitor. In some embodiments, an immune checkpoint inhibitor includes an agent that inhibits CTLA-4, PD-1, PD-L1, and the like. Suitable anti-CTLA-4 inhibitors include, for example, ipilimumab, tremelimumab, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B 1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin. Oncology, 22(145): Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Suitable anti-PD-1 inhibitors, include, for example, nivolumab, pembrolizumab, pidilizumab, MED10680, and combinations thereof. In other specific embodiments, anti-PD-L1 therapy agents include atezolizumab, BMS-936559, MEDI4736, MSB0010718C, and combinations thereof.

Suitable anti-PD-1 inhibitors include, for example, those described in Topalian, et al., Immune Checkpoint Blockade: A Common Denominator Approach to Cancer Therapy, Cancer Cell 27: 450-61 (Apr. 13, 2015), incorporated herein by reference in its entirety.

In some embodiments, the checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, provided is a method of improving treatment outcome in a subject receiving immunotherapy. The method generally includes administering an effective amount of an immunotherapy to the subject having cancer; and administering a therapeutically effective amount of a CD70 conjugate or a pharmaceutical composition thereof to the subject, wherein the CD70 conjugate thereof specifically binds to CD70+ cancer cells; wherein the treatment outcome of the subject is improved, as compared to administration of the immunotherapy alone. In some embodiments, the CD70 conjugate comprises any of the embodiments of CD70 conjugates as described herein.

In some embodiments, an improved treatment outcome is an objective response selected from stable disease, a partial response or a complete response as determined by standard medical criteria for the cancer being treated. In some embodiments, an improved treatment outcome is reduced tumor burden. In some embodiments, an improved treatment outcome is progression-free survival or disease-free survival.

### Treatment of Autoimmune Disease

In some embodiments, the CD70 conjugates as described herein can be used in a method(s) comprising administering a CD70 conjugate as described herein to a subject in need thereof, such as a subject having an autoimmune disease.

In some embodiments, provided are methods of treating an autoimmune disease comprising administering a CD70 conjugate comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:3 and SEQ ID NO:4, respectively. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:5 and SEQ ID NO:6, respectively. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:7 and SEQ ID NO:8, respectively. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:9 and SEQ ID NO:10, respectively. In some embodiments, provided are methods of treating an autoimmune disease comprising administering a CD70 conjugate comprising a heavy chain variable region (VH) and a light chain variable region (VL), the VH and VL regions having amino acid sequences set forth in and SEQ ID NO:11 and SEQ ID NO:12; respectively.

In some embodiments, provided are methods of treating an autoimmune disease comprising administering a CD70 conjugate comprising a heavy chain variable region (VH) and a light chain variable region (VL), the VH and VL regions having amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified.

In some embodiments, provided are methods of treating an autoimmune disease comprising administering a CD70 conjugate comprising a heavy chain variable region (VH) and a light chain variable region (VL), the VH and VL regions having the amino acid sequences set forth in the pairs of amino acid sequences selected from SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; and SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In some embodiments, provided are methods of treating an autoimmune disease comprising administering a CD70 conjugate comprising a heavy chain variable region (VH) and a light chain variable region (VL), the VH and VL regions having the amino acid sequences set forth in SEQ ID NO:11 and SEQ ID NO:12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO:21, SEQ ID NO:22, SEQ ID NO:13, SEQ ID NO:24, SEQ ID NO:25 and SEQ ID NO:26, respectively; (ii) SEQ ID NO:21, SEQ ID NO:22, SEQ ID NO:14, SEQ ID NO:24, SEQ ID NO:25 and SEQ ID NO:26, respectively; (iii) SEQ ID NO:21, SEQ ID NO:22, SEQ ID NO:15, SEQ ID NO:24, SEQ ID NO:25 and SEQ ID NO:26, respectively; (iv) SEQ ID NO:21, SEQ ID NO:22, SEQ ID NO:23, SEQ ID NO:24, SEQ ID NO:25 and SEQ ID NO:18, respectively; and (v) SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:13, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:14, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:15, SEQ ID NO:24, SEQ ID NO:25 and SEQ ID NO:26, respectively. In some embodiments, each VH and VL region comprises a humanized framework region. In some embodiments, each VH and VL region comprises a human framework region.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:21, SEQ ID NO:22, SEQ ID NO:23, SEQ ID NO:24, SEQ ID NO:25 and SEQ ID NO:18, respectively. In some embodiments, each VH and VL region comprises a humanized framework region. In some embodiments, each VH and VL region comprises a human framework region.

In some embodiments, provided are methods of treating an autoimmune disease comprising administering a CD70 conjugate comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:16, SEQ ID NO:17, SEQ ID NO:23, SEQ ID NO:24, SEQ ID NO:25 and SEQ ID NO:26; respectively. In some embodiments, each VH and VL region comprises a humanized framework region. In some embodiments, each VH and VL region comprises a human framework region.

In some embodiments, the subject is in need of treatment for an autoimmune disease. The methods described herein include administering a therapeutically effective amount of a CD70 conjugate to a subject having an autoimmune disease. As used herein, the phrase “therapeutically effective amount”, “effective amount” or “effective dose” refers to an amount of the CD70 conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by expression of CD70 by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

In some embodiments, the immunological disorder is a T cell-mediated immunological disorder, such as a T cell disorder in which activated T cells associated with the disorder express CD70. CD70 conjugates can be administered to deplete such CD70-expressing activated T cells. In a specific embodiment, administration of CD70 conjugates can deplete CD70-expressing activated T cells, while resting T cells are not substantially depleted by the anti-CD70 conjugates. In this context, “not substantially depleted” means that less than about 60%, or less than about 70% or less than about 80% of resting T cells are not depleted.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in CD70+ autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a CD70 conjugate as described herein into a subject by a method or route which results in binding to the CD70 conjugate to CD70+ autoimmune cells. Similarly, a pharmaceutical composition comprising a CD70 conjugate as described herein can be administered by any appropriate route which results in an effective treatment in the subject.

The dosage ranges for a CD70 conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a CD70 conjugate can be administered in a unit dose. The term “unit dose” when used in reference to a pharmaceutical composition refers to physically discrete units suitable as unitary dosage for the subject, each unit containing a predetermined quantity of active material (e.g., a CD70 conjugate), calculated to produce the desired therapeutic effect in association with the required physiologically acceptable diluent, i.e., carrier, or vehicle.

In some embodiments, a CD70 conjugate, or a pharmaceutical composition thereof, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a CD70 conjugate or a pharmaceutical composition thereof to the subject, wherein the CD70 conjugate specifically binds to CD70+ autoimmune cells; wherein the treatment outcome of the subject is improved, as compared to administration of the immunotherapy alone. In some embodiments, the CD70 conjugate comprises any of the embodiments of CD70 conjugates as described herein. In some embodiments, an improved treatment outcome is a decrease in disease progression, an alleviation of one or more symptoms, or the like.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### General Methods

¹H NMR and other NMR spectra were recorded on Bruker AVIII 400 or Bruker AVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 um; Column Temperature: 40° C. Detectors: ADC ELSD, DAD (214 nm and 254 nm), ES-API.

Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.

Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.

Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:

Method A: Waters SunFire 10 μm C18 column (100 \acute{Å}, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.

Method B: Waters SunFire 10 μm C18 column (100 \acute{Å}, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.

Method C: Waters Xbridge 10 μm C18 column (100 \acute{Å}, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of a Sugar Unit

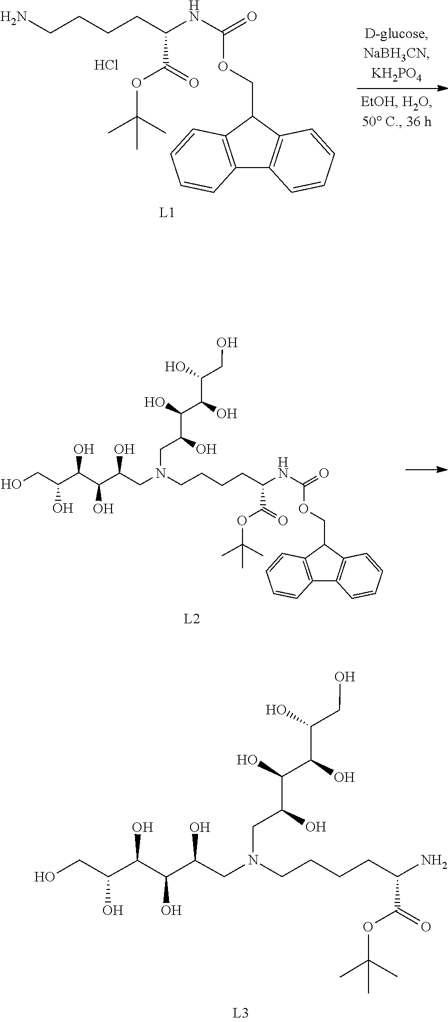 [SMILES: CC(C)(C)OC(=O)[C@H](CCCCN)NC(=O)OCC1c2ccccc2-c2ccccc21 ; Cl ; CC(C)(C)OC(=O)[C@H](CCCCN(C[C@H](O)[C@@H](O)[C@H](O)[C@H](O)CO)C[C@H](O)[C@@H](O)[C@H](O)[C@H](O)CO)NC(=O)OCC1c2ccccc2-c2ccccc21 ; CC(C)(C)OC(=O)[C@@H](N)CCCCN(C[C@H](O)[C@@H](O)[C@H](O)[C@H](O)CO)C[C@H](O)[C@@H](O)[C@H](O)[C@H](O)CO]

A Sugar unit was prepared as follows:

Step 1 A reaction mixture of compound L1 (5 g, 10.846 mmol), D-glucose (19.54 g, 108.460 mmol), NaBH₃CN (5.45 g, 86.768 mmol) and potassium dihydrogen phosphate (0.379 mL, 6.508 mmol) in water (40 mL) and ethanol (65 mL) was stirred at 50° C. under N₂ for 36 hr, until the reaction was complete as indicated by LCMS. The solvents were evaporated, and the residue was purified by C18 reversed-phase chromatography to give the desired product L2 (3.5 g, 4.649 mmol, 42.86%). LCMS (M+H)⁺=753.0;

1H NMR (400 MHz, DMSO) δ 7.90 (d, J=7.5 Hz, 2H), 7.74-7.64 (m, 2H), 7.44-7.32 (m, 4H), 4.58-4.21 (m, 8H), 4.14-3.74 (m, 4H), 3.68-3.41 (m, 8H), 2.85-2.56 (m, 2H), 1.69-1.28 (m, 15H). 13C NMR (100 MHz, DMSO) δ 171.53, 156.10, 143.77, 140.70, 127.62, 127.04, 125.24, 120.10, 80.47, 80.42, 71.66, 71.58, 71.34, 70.18, 65.53, 63.51, 63.36, 54.48, 54.41, 46.63, 27.65, 23.14, 22.38.

Step 2 To a solution of L2 (200 mg, 0.266 mmol) in THF (2 mL) was added the diethylamine (38.86 mg, 0.531 mmol). The reaction mixture was stirred at room temperature for 2 hr. A sample was taken from the reaction mixture, and the LCMS result showed the desired product was found and the starting material was consumed completely. The solvents were evaporated, and the residue was purified by C18 reversed-phase chromatography to give the desired product L3 (120 mg), LCMS (M+H)⁺=531.1.

### Example 2: Preparation of a PEG Unit

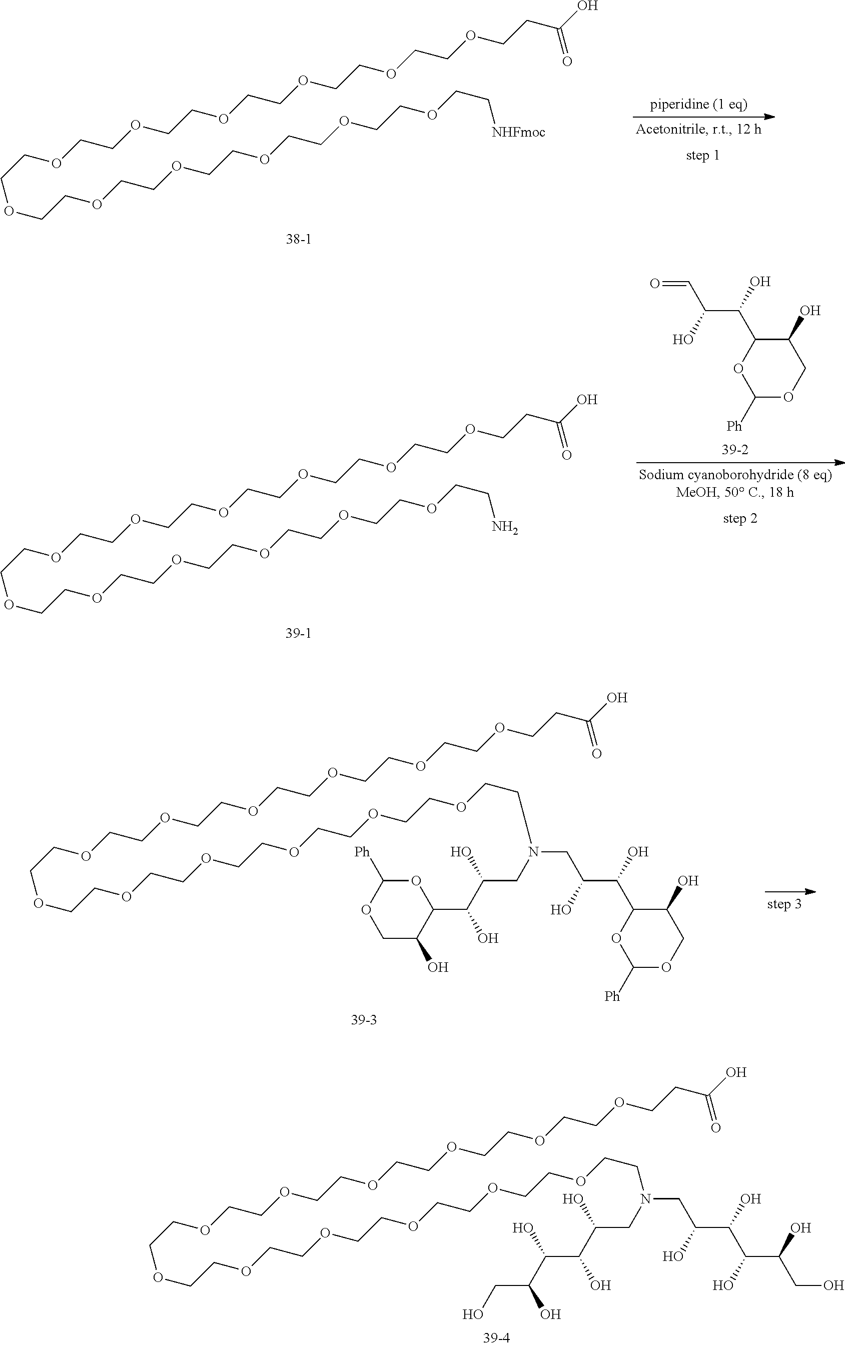

A PEG unit containing linear monosaccharide was prepare as follows:

### Step 1

A solution of compound 38-1 (260 mg, 0.31 mmol) in acetonitrile (3.0 mL) was stirred at r.t. and diethyl amine anhydrous (0.2 mL, 1.941 mmol) was added. The resulting solution was stirred at r.t. for 2 h until LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to give expected fractions of compound 39-1 (170 mg, 0.28 mmol) as a pale yellow oil. LCMS, ESI m/z=618.4 (M+H)⁺;

### Step 2

A clear reaction solution of compound 39-1 (170 mg, 0.28 mmol), 39-2 (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and compound 39-2 (217.08 mg, 1.206 mmol) was added and kept stirring at 50° C. overnight. After stirring for 20 hrs, LCMS indicated the reaction was complete. The solvents were evaporated, and the residue was purified by C18 reversed-phase chromatography to give the desired product 39-3 (265 mg, 0.24 mmol). LCMS, ESI m/z=1122.6 (M+H)⁺.

### Step 3

A mixture of compound 39-3 (265 mg, 0.24 mmol) in 6N HCl/THF aqueous was stirred at rt for 3 hours, until LCMS indicated the reaction was complete. The solvents were neutralized with NaHCO₃ aqueous, evaporated, and the residue was purified by C18 reversed-phase chromatography to give the desired product 39-4 (160 mg, 0.17 mmol). LCMS, ESI m/z=946.5 (M+H)⁺.

### Example 3: Preparation of a Drug-Linker (PB003) Containing MMAE

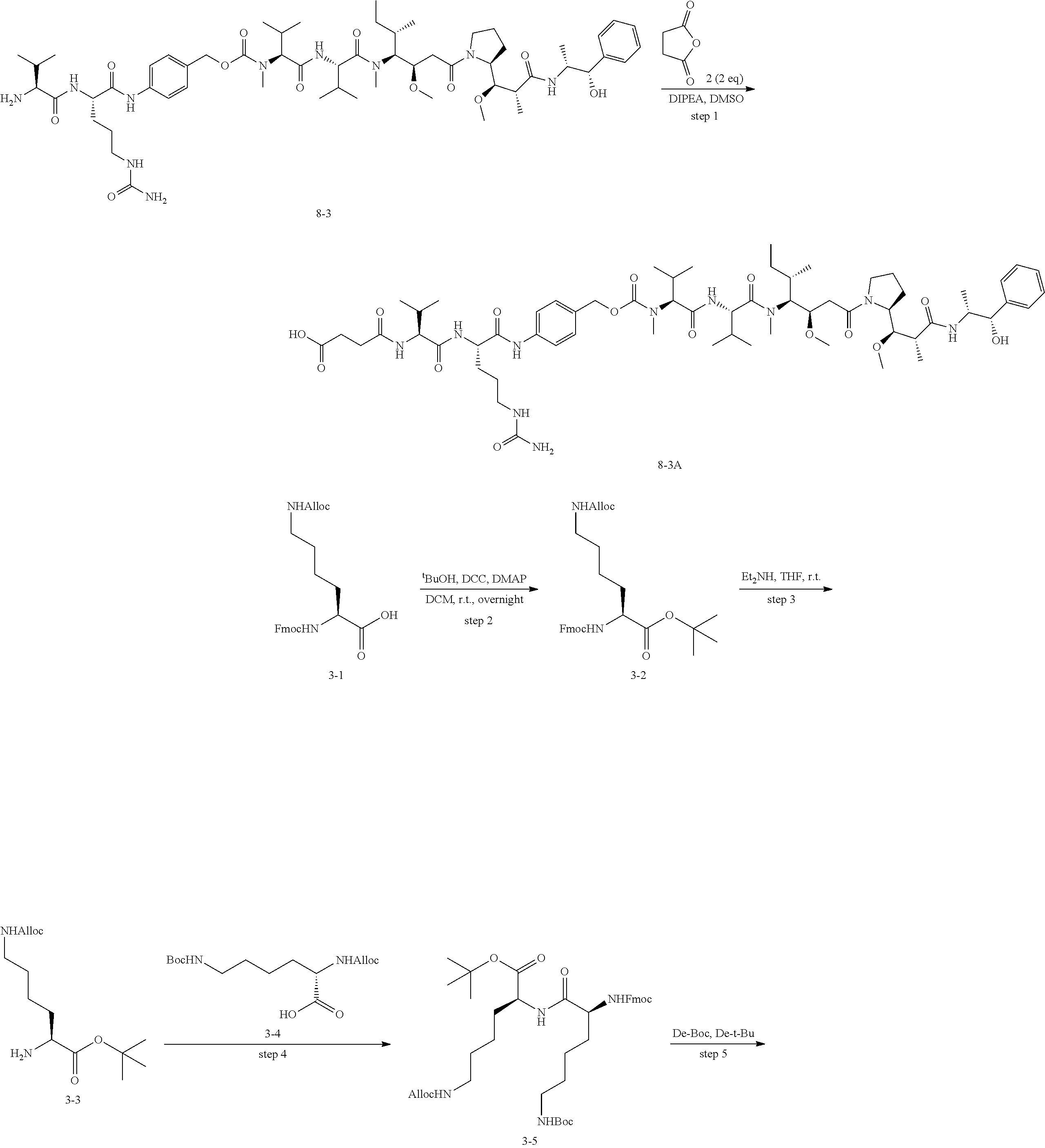

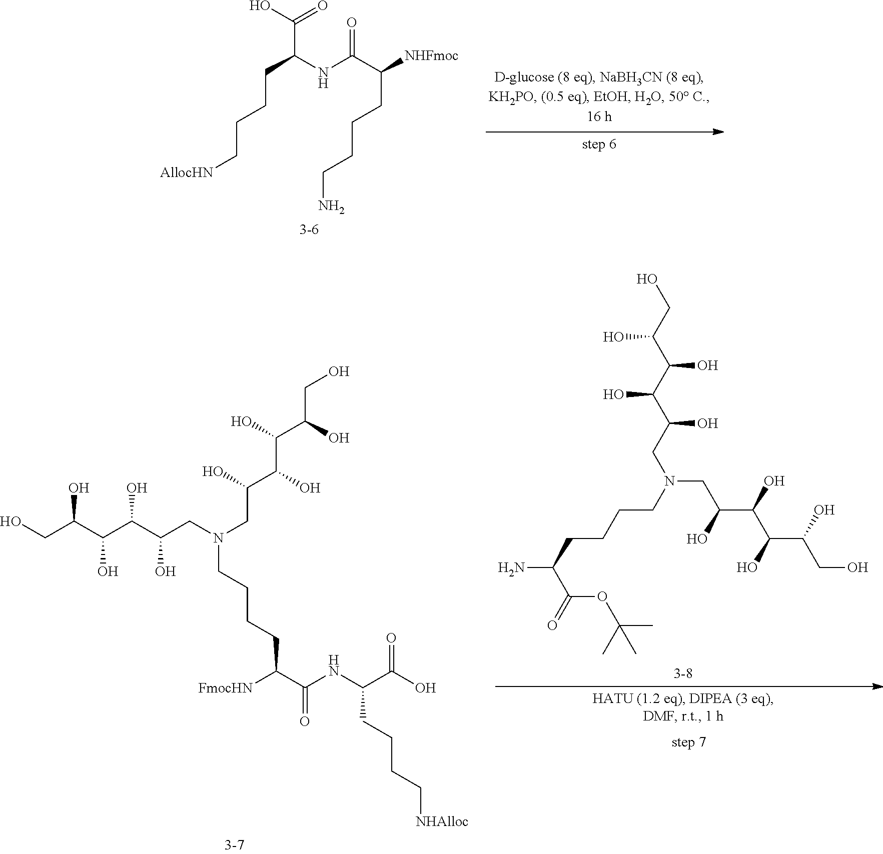

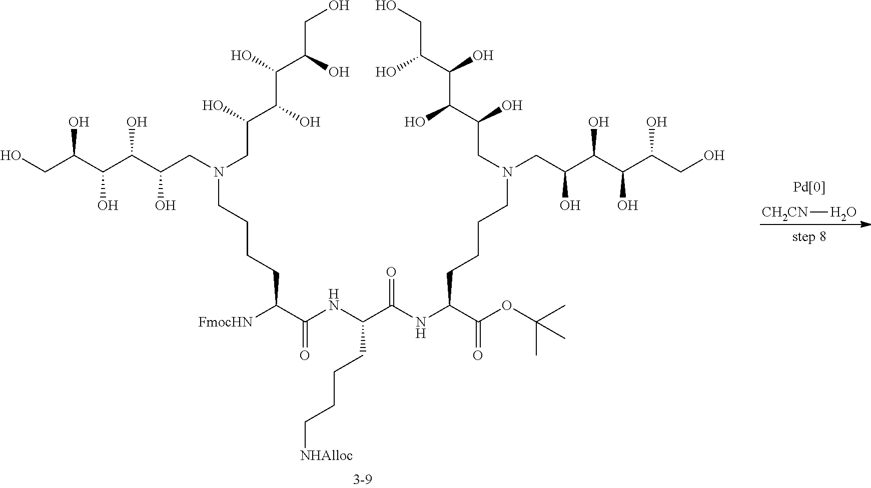

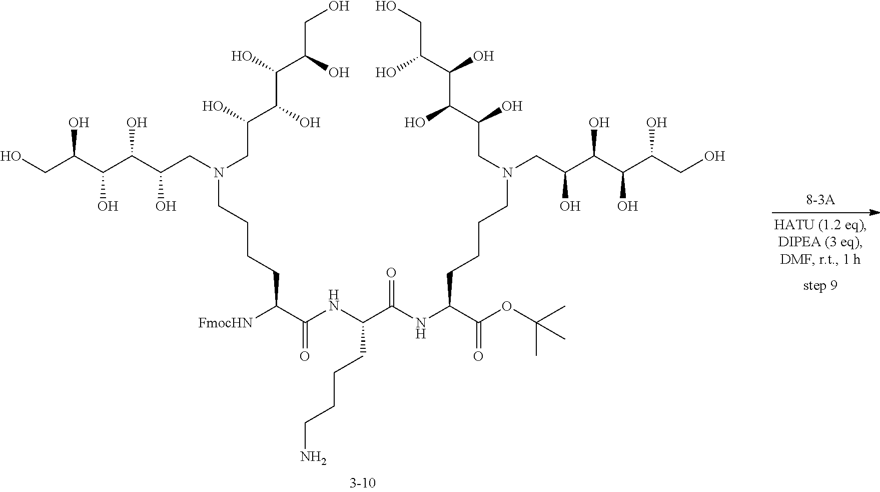

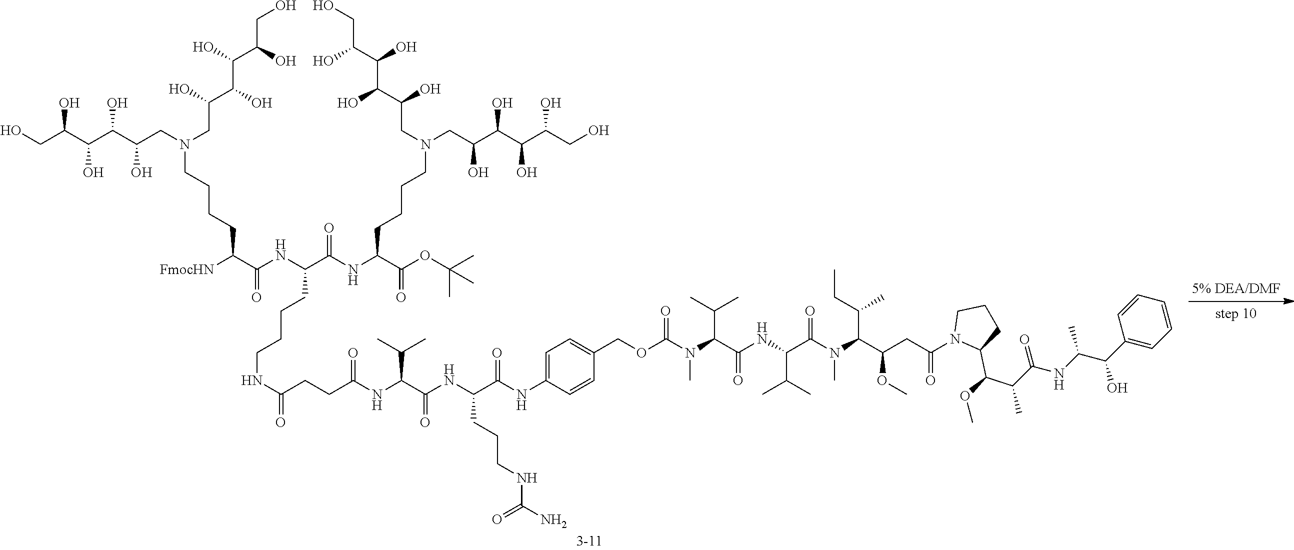

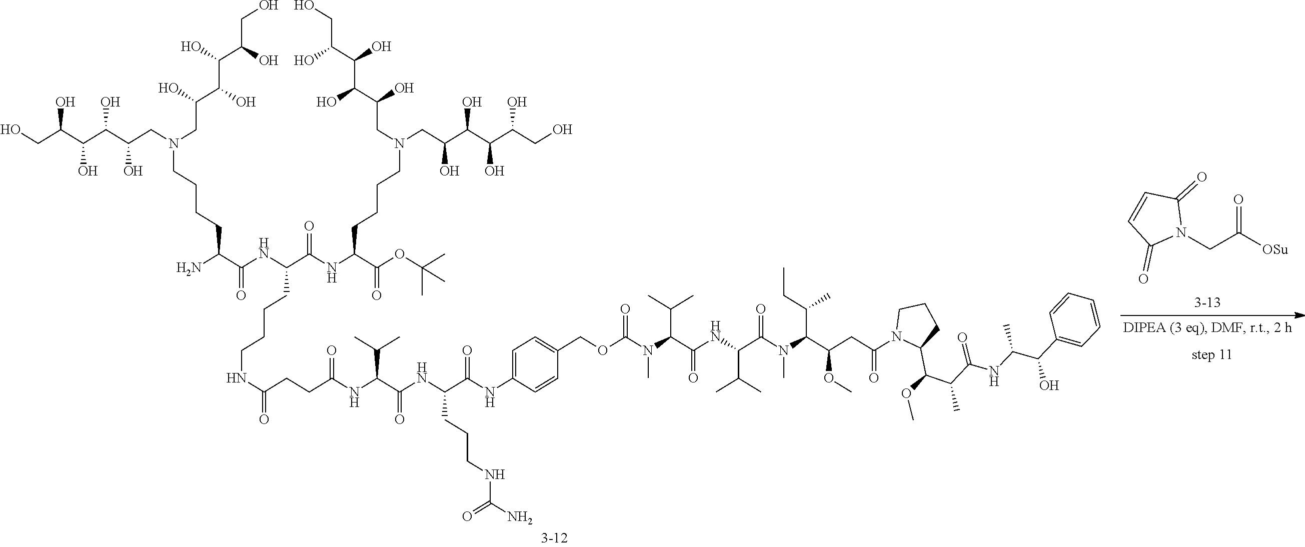

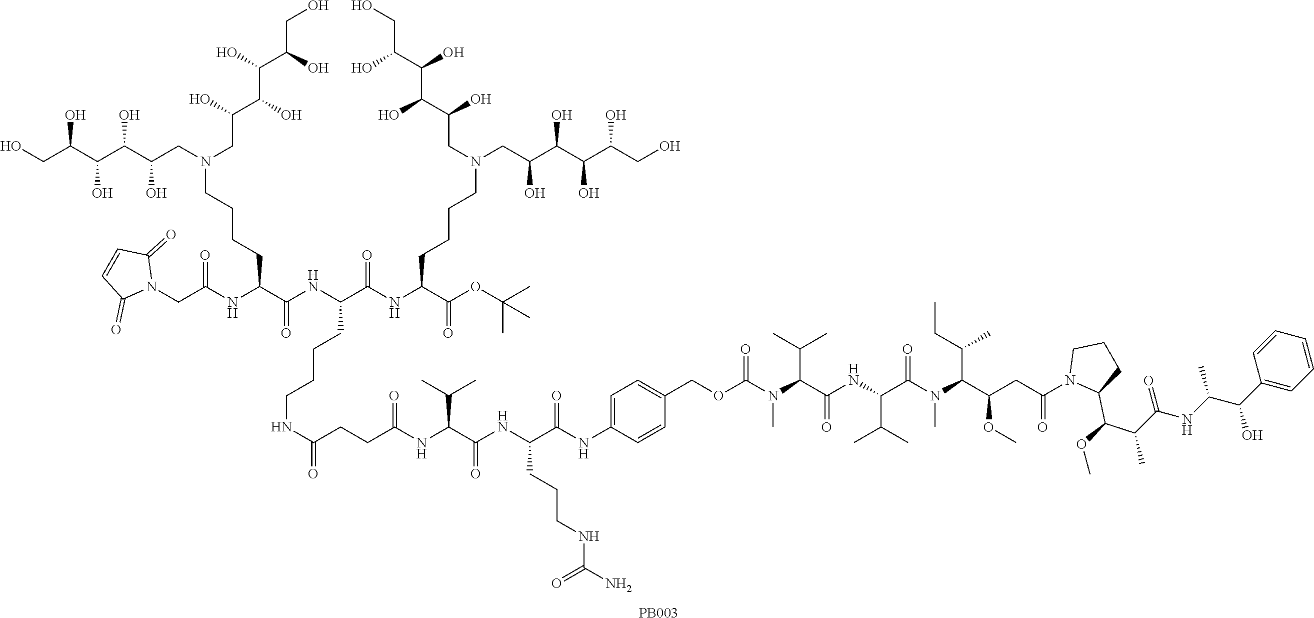

A Drug Linker containing two Sugar units and a cleavable linker attached to MMAE (PB003) was prepared as follows:

### Step 1

A solution of compound 8-3 (30 mg, 0.027 mmol), DIPEA (10.45 mg, 0.081 mmol) in anhydrous DMF (2 mL) was stirred at room temperature, then oxolane-2,5-dione (5.40 mg, 0.054 mmol) was added. The resulting solution was stirred for another 1 hr at room temperature (r.t.) until liquid chromatography mass spectrometry (LCMS) indicated a complete reaction. The mixture was purified directly by reverse phase liquid chromatography (40 g C18 column, eluting with 0-50% acetonitrile in water with 0.01% TFA over 15 min) to give compound 8-3A (25.8 mg, 0.021 mmol, 78.97%) as a white solid. LCMS: Product (M/2+H)⁺=612.5;

### Step 2

A mixture of compound 3-1 (20.00 g, 44.198 mmol), 2-methylpropan-2-ol (12.600 mL, 132.594 mmol), DCC (13.68 g, 66.297 mmol) and DMAP (1.62 g, 13.259 mmol) in DCM (150 L) was stirred for 12 hr at room temperature (r.t.) under nitrogen (N₂). After completion of the reaction as judged by LCMS, the reaction mixture was filtered through a Celite pad, and the filtrate was concentrated under the reduced pressure to give a residue which was then purified by silicon-gel flash chromatography (Petroleum Ether:EtOAc=10:1) to afford the compound 3-2 (11.80 g, 23.200 mmol, 52.49%) as a colorless oil.

LCMS (M−56+H)⁺=453.1;

1H NMR (400 MHz, DMSO) δ=7.89 (d, J=7.5, 2H), 7.72 (d, J=7.5, 2H), 7.65 (d, J=7.8, 1H), 7.44 (t, J=7.4, 2H), 7.35 (d, J=7.1,

2H), 7.20 (t, J=5.3, 1H), 5.94-5.76 (m, 1H), 5.27 (dd, J=17.2, 1.6, 1H), 5.16 (dd, J=10.5, 1.4, 1H), 4.45 (d, J=5.3, 2H), 4.32 (dd, J=12.3, 4.8, 2H), 4.26-4.20 (m, 1H), 3.84 (d, J=4.9, 1H), 2.97 (dd, J=12.6, 6.4, 2H), 1.66-1.53 (m, 2H), 1.38 (s, 9H), 1.33-1.29 (m, 2H), 1.28-1.23 (m, 2H).

### Step 3

To a solution of compound 3-2 (5 g, 9.837 mmol) in DCM (20 mL) was added Et2NH (4 mL, 38.693 mmol). The reaction was stirred at room temperature for 2 h. The mixture was concentrated and the crude compound 3-3 (2.84 g, 9.921 mmol, 100%) was used in the next step directly. ESI m/z: 287.3 (M+H)⁺.

### Step 4

To a solution of compound 3-3 (2.84 g, 9.917 mmol) in DMF (15 mL) was added compound 3-4 (5.58 g, 11.901 mmol), DIPEA (2.56 g, 19.834 mmol) and HATU (3.77 g, 9.917 mmol). The reaction was stirred at room temperature for 1 h. Then the mixture was concentrated and purified by reverse phase separation (C18 column, eluting with 0-87% acetonitrile in water with TFA) to afford the compound 3-5 (5.2 g, 7.056 mmol, 71.15%) as white solid. ESI m/z: 759.4 (M+Na)⁺.

### Step 5

To a solution of compound 3-5 (5.2 g, 7.056 mmol) in DCM (12 mL) was added TFA (12 mL, 1199.474 mmol). The reaction was stirred at room temperature for 4 h. Then the mixture was concentrated and purified by reverse column separation (C18 column, eluting with 0-44% acetonitrile in water with TFA) to yield compound 3-6 (2.4 g, 4.133 mmol, 58.57%) as a white solid. ESI m/z: 581.3 (M+H)⁺.

### Step 6

To a solution of compound 3-6 (2.40 g, 4.133 mmol) in EtOH (35 mL) and H₂O (5 mL) was added D-glucose (5.93 g, 32.919 mmol), KH₂PO₄ (0.020 mL, 0.344 mmol) and NaBH₃CN (2.08 g, 33.099 mmol). The reaction was stirred at 50° C. for 18 h. The reaction was stirred at room temperature for 4 h. Then the mixture was concentrated and purified by reverse column separation (C18 column, eluting with 0-44% acetonitrile in water with TFA) to yield compound 3-7 (2.0 g, 2.200 mmol, 53.48%) as white solid. ESI m/z: 910.4 (M+H)⁺.

### Step 7

To a solution of compound 3-7 (1.00 g, 1.100 mmol) in DMF (15 mL) was added HATU (0.50 g, 1.320 mmol) and DIPEA (0.43 g, 3.300 mmol). The mixture was stirred for 10 min and then compound 3-8 (0.58 g, 1.099 mmol) was added. The reaction was stirred for 1 h at room temperature. Then the mixture was concentrated and purified by reverse phase column separation (C18 column, eluting with 0-34% acetonitrile in water with TFA) to yield compound 3-9 (0.47 g, 0.334 mmol, 30.37%). ESI m/z: 711.5 (M/2+H)⁺.

### Step 8

A clear solution of compound 3-9 (10 mg, 0.007 mmol), Pd(PPh3)4 (0.41 mg, 0.000 mmol) and diethyl amine anhydrous (0.001 mL, 0.014 mmol) in MeCN (0.5 mL) and water (0.1 mL) was stirred at room temperature for 2 hr under N₂. LCMS indicated all starting material was consumed, and desired mass of product (fragment mass 669 in LCMS) was detected. The mixture was purified by reverse phase liquid chromatography (12 g C18 column, eluting with 0-35% acetonitrile in water with 0.01% TFA) to yield product 3-10 as a white solid. LCMS: Product (M/2+H)⁺=669.5; purity 65% (214 nm).

### Step 9

A solution of compound 3-10 (27 mg, 0.024 mmol), 8-3A (54.67 mg, 0.048 mmol) and DIPEA (3.10 mg, 0.024 mmol) in anhydrous DMF (1.8 mL) was stirred at room temperature for 5 min, then a solution of HATU (9.14 mg, 0.024 mmol) in anhydrous DMF (0.2 mL) was added. The resulting solution was stirred for another 1 hr at r.t. until LCMS indicated complete reaction. The reaction mixture was purified directly by reverse phase liquid chromatography (40 g C18 column, eluting with 0-70% acetonitrile in water with 0.01% TFA over 15 min) to yield product 3-11 (40.5 mg, 0.016 mmol) as a white solid. LCMS (M/3+H)⁺=848.5;

### Step 10

A solution of compound 3-11 (45 mg, 0.018 mmol) in DMF (0.95 mL) was stirred at r.t. and diethyl amine anhydrous (0.05 mL, 0.485 mmol) was added. After addition, the resulting solution was kept stirring at rt. for another 1 hr until LCMS showed that the reaction was completed. Volatiles (especially DEA) were evaporated off to give a crude product, purified directly by reverse phase liquid chromatography (12 g C18 column, eluting with 0-50% acetonitrile in water with 0.01% TFA over 15 min) to give expected fractions, which were lyophilized to yield product 3-12 (30 mg, 0.013 mmol, 73.05%) as a white solid. LCMS (M/3+H)⁺=774.4;

### Step 11

A solution of compound 3-12 (20 mg, 0.009 mmol) and DIPEA (3.34 mg, 0.026 mmol) in anhydrous DMF (0.8 mL) was stirred at room temperature for 5 min, then a solution of 2,5-dioxopyrrolidin-1-yl 2-(2,5-dioxo-2,5-dihydro-1H-pyrrol-1-yl)acetate (4.35 mg, 0.017 mmol) in anhydrous DMF (0.2 mL) was added dropwise over 2 min. The resulting solution was stirred for another 2 hr at r.t., then monitored by LCMS; the desired product was formed as majority of the reaction product. The reaction was quenched with one drop of water and then purified directly by Prep-HPLC (Mobile Phase: A: Water (0.01% TFA) B: acetonitrile (0.01% TFA); Gradient Phase: 5% of B increasing to 95% of B with 15 min; Flow Rate: 1.0 mL/min; Column: SunFire C18, 4.6*50 mm, 3.5 m; Column Temperature: 50° C. Detectors: ADC ELSD, DAD (214 nm and 254 nm) to yield product PB003 (7 mg, 0.003 mmol) as a white solid. LCMS (M/3+H)⁺=820.1

1H NMR (400 MHz, DMSO-d6) δ 9.86-9.78 (m, 1H), 8.49-8.31 (m, 3H), 8.26-8.06 (m, 3H), 8.01-7.83 (m, 3H), 7.66-7.59 (m, 2H), 7.32-7.25 (m, 6H), 7.20-7.15 (m, 1H), 7.09 (s, 1H), 6.05-5.98 (m, 1H), 5.45-5.35 (m, 6H), 5.13-4.95 (m, 2H), 4.87-4.71 (m, 4H), 4.71-4.41 (m, 13H), 4.35-4.13 (m, 7H), 4.10-4.04 (m, 2H), 3.99-3.94 (m, 6H), 3.80-3.77 (m, 1H), 3.73-3.69 (m, 4H), 3.67-3.43 (m, 21H), 3.24-3.17 (m, 17H), 3.12-2.97 (m, 7H), 2.89-2.83 (m, 3H), 2.44-2.24 (m, 7H), 2.16-1.91 (m, 5H), 1.84-1.42 (m, 19H), 1.38 (s, 9H), 1.34-1.24 (m, 9H), 1.06-0.98 (m, 6H), 0.89-0.75 (m, 26H) ppm. Proton signals of two molecule of TFA are included.

Drug linker PB003 can be used to make a conjugate such as PA003.

### Example 4: Preparation of the Drug-Linker (PB004) Having Two Sugar Units and a Cleavable Linker Attached to MMAE

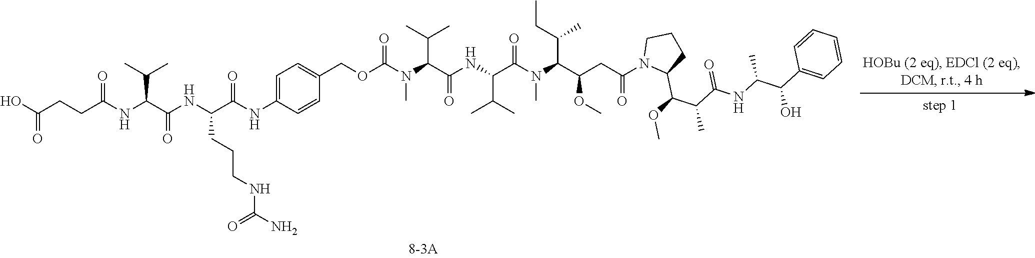 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCC(=O)O)C(C)C)cc1)C(C)C]

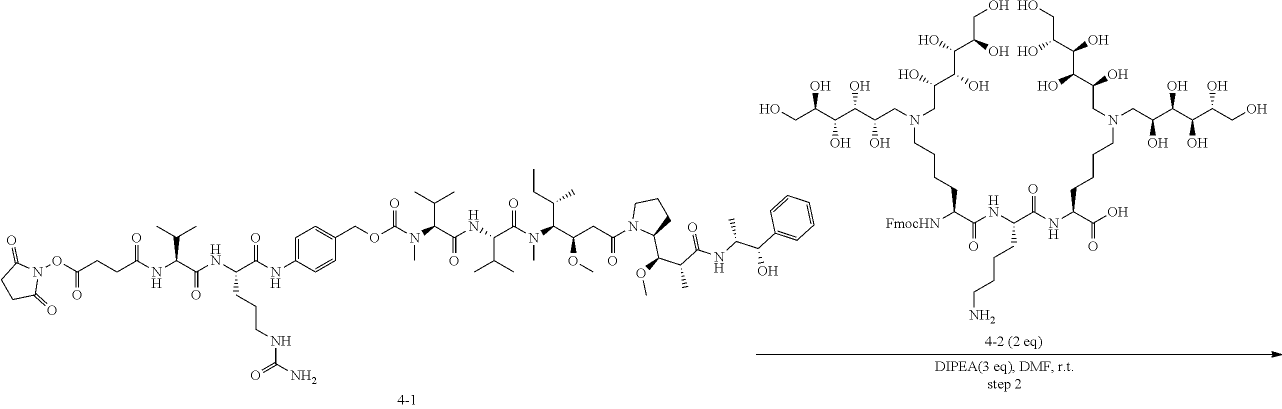

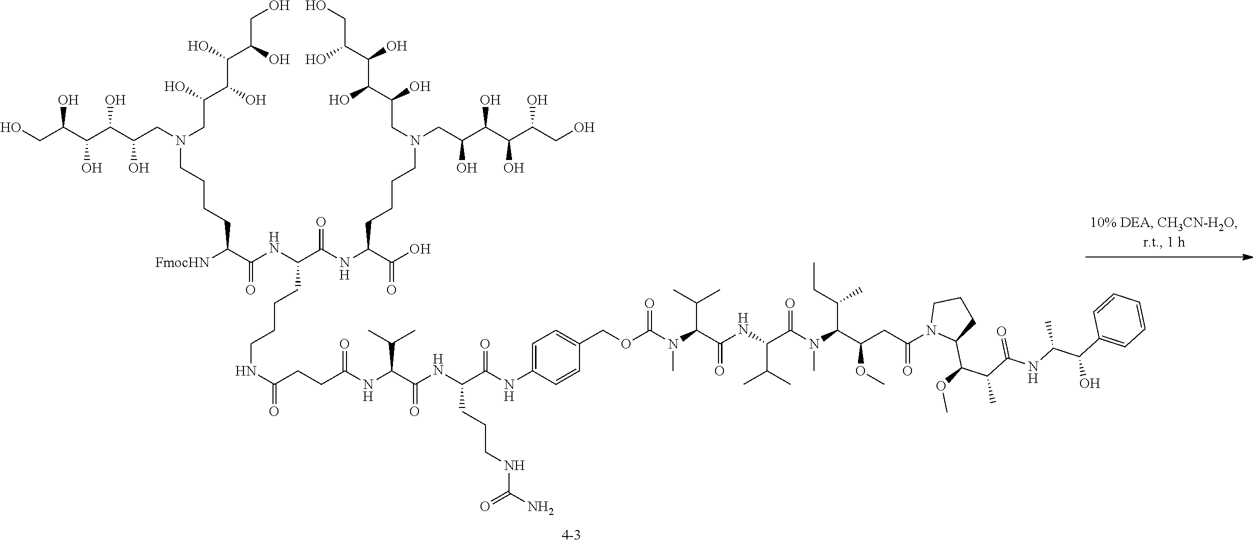

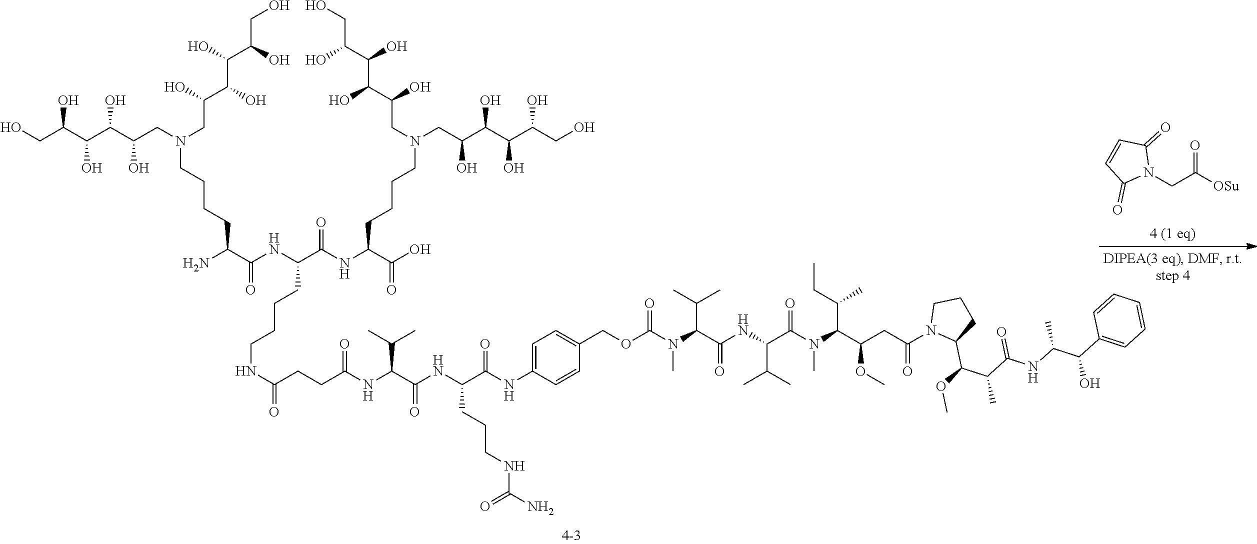

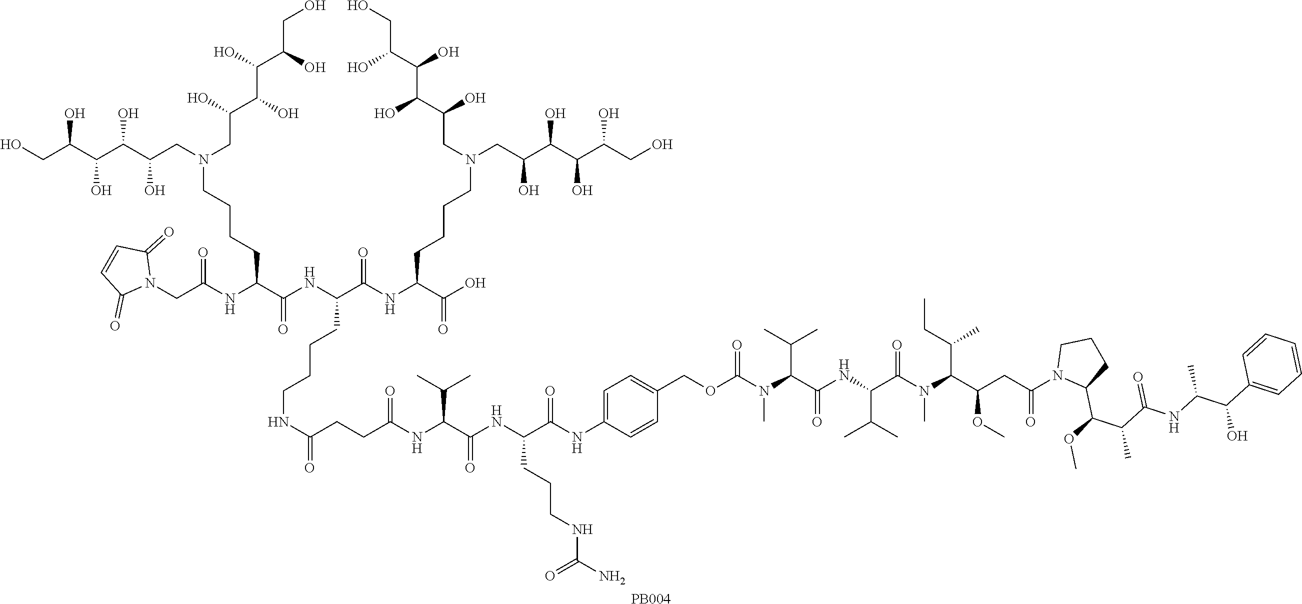

A Drug Linker containing Sugar units and a cleavable linker attached to MMAE (PB004) was prepared as follows:

### Step 1

A solution of compound 8-3A (50 mg, 0.041 mmol) and HOSu (7.05 mg, 0.061 mmol) in anhydrous DCM (5 mL) was stirred at room temperature, and then a solution of EDCI (11.75 mg, 0.061 mmol) was added. The resulting solution was stirred for another 1.5 hr at r.t. until LCMS indicated a complete reaction. The reaction solution was diluted with more DCM (10 mL) and washed with water. The organic layer was collected and dried over sodium sulfate, then condensed to dryness to give crude NHS ester 4-1 (50 mg, 0.038 mmol, 92.65%) as a white solid, which was used directly in next step (refer to N200897-071). LCMS (M/2+H)⁺=661.0; purity=96% (254 nm).

### Step 2

The crude compound 4-1 (50 mg, 0.038 mmol) from last step was dissolved in anhydrous DMF (2 mL), then DIPEA (14.65 mg, 0.114 mmol) and compound 4-2 (48.52 mg, 0.038 mmol) was added. The resulting solution was stirred at room temperature for 2 h until LCMS indicated all starting amine was consumed. The reaction solution was purified directly by reverse phase liquid chromatography (40 g C18 column, eluting with 0-70% acetonitrile in water with 0.01% TFA over 15 min) to yield compound 4-3 (50 mg, 0.020 mmol, 53.10%) as a white solid. LCMS: m/z=829.8 (M/3+H)⁺;

### Step 3

A suspension of compound 4-3 (50 mg, 0.022 mmol) in acetonitrile (2 mL) was stirred at r.t. and water (0.5 mL) was added to improve the solubility. The material was dissolved into a clear solution. Then diethyl amine anhydrous (0.2 mL, 1.941 mmol) was added. The resulting solution was stirred at r.t. for 1 h. until the LCMS showed that the reaction was completed. Then the solution was concentrated to dryness to remove most of the diethyl amine, and then redissolved in acetonitrile and water and lyophilized to yield crude product 4-4 (48 mg, 0.021 mmol, 96.94%) as a white solid, which was used directly in the next step. LCMS: m/z=739.0 (inclusive fragment piece, (2263/3)+H=755 is expected);

### Step 4

A solution of compound 4-4 (47 mg, 0.021 mmol) and DIPEA (8.03 mg, 0.062 mmol) in anhydrous DMF (0.8 mL) was stirred at room temperature for 5 min, then a solution of 2,5-dioxopyrrolidin-1-yl 2-(2,5-dioxo-2,5-dihydro-1H-pyrrol-1-yl)acetate (5.23 mg, 0.021 mmol) in anhydrous DMF (0.2 mL) was added dropwise by syringe. The resulting solution was stirred for another 0.5 hr at r.t. until LCMS indicated all starting amine was consumed and the mass of desired product was detected. The resulting solution was stirred at room temperature for 2 h until LCMS indicated all starting amine was consumed. The reaction solution was purified directly by Prep-HPLC (eluting with gradient with 0.01% TFA over 20 min) to yield product PB004 (10 mg, 0.004 mmol, 20.06%) as a white solid. LCMS: m/z=801.4 (M/3+H)⁺;

1H NMR (400 MHz, DMSO-d6) δ 12.56 (s, 1H), 9.77 (s, 1H), 8.52-8.23 (m, 3H), 8.19-7.84 (m, 6H), 7.67-7.61 (m, 2H), 7.35-7.24 (m, 6H), 7.19-7.13 (m, 1H), 7.09 (s, 2H), 6.05-6.02 (m, 1H), 5.52-5.36 (m, 6H), 5.13-4.94 (m, 2H), 4.89-4.73 (m, 4H), 4.69-4.36 (m, 13H), 4.29-4.23 (m, 6H), 4.14-4.09 (m, 2H), 4.05-3.93 (m, 6H), 3.79-3.76 (m, 1H), 3.72-3.68 (m, 4H), 3.65-3.43 (m, 21H), 3.24-3.05 (m, 17H), 3.01-2.91 (m, 7H), 2.89-2.83 (m, 3H), 2.44-2.23 (m, 7H), 2.14-1.94 (m, 5H), 1.86-1.41 (m, 19H), 1.37-1.20 (m, 9H), 1.05-0.97 (m, 6H), 0.88-0.75 (m, 26H) ppm. Proton signals of two molecule of TFA are included. Drug linker PB004 can be used to make a conjugate such as PA004.

### Example 5: Preparation of the Drug-Linker (PB008) Having One Sugar Unit and a Cleavable Linker Attached to MMAE

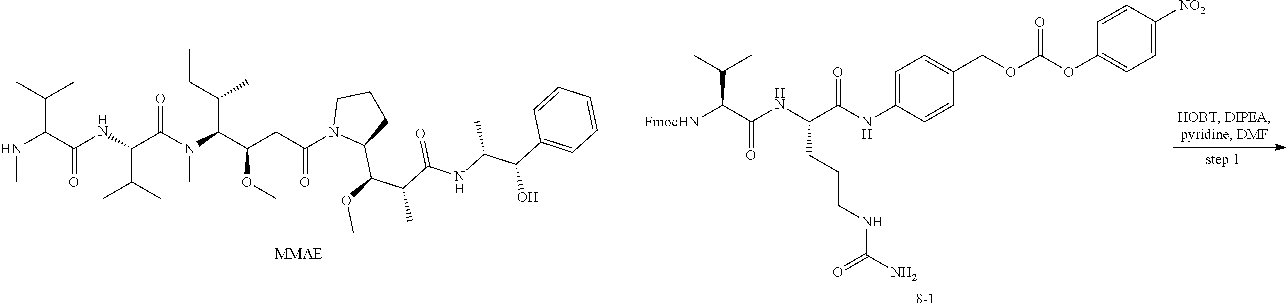

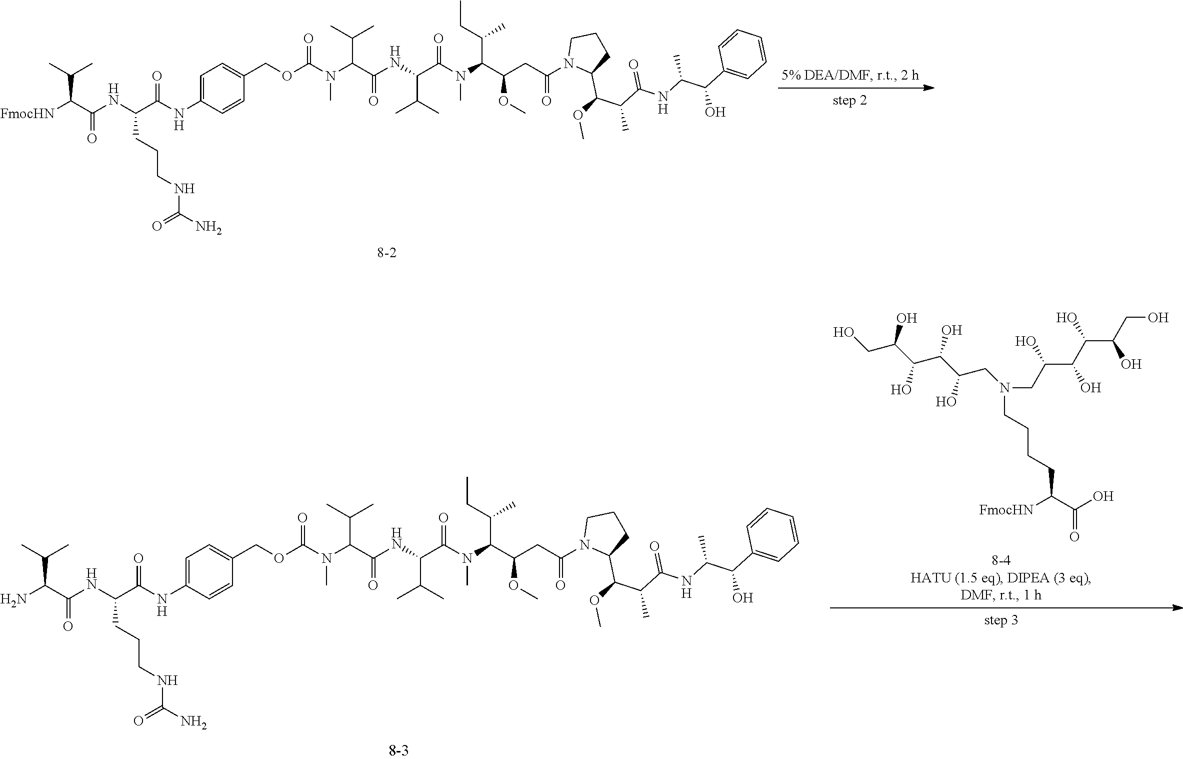

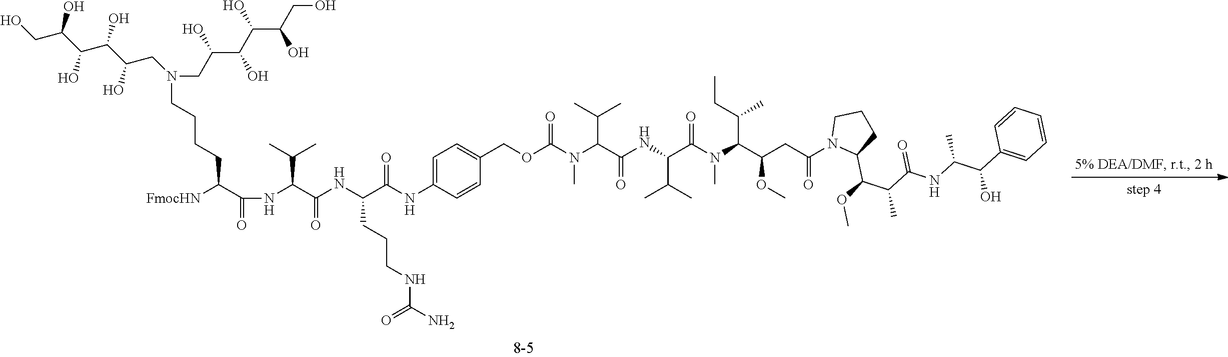

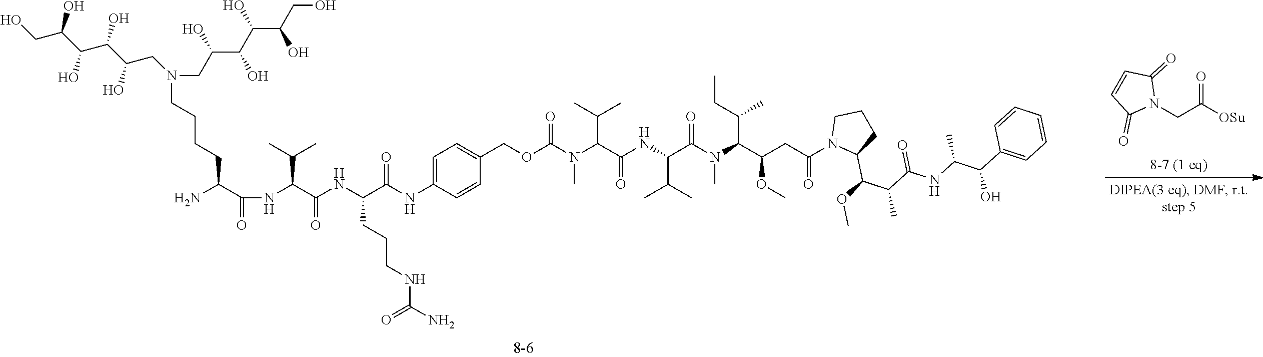

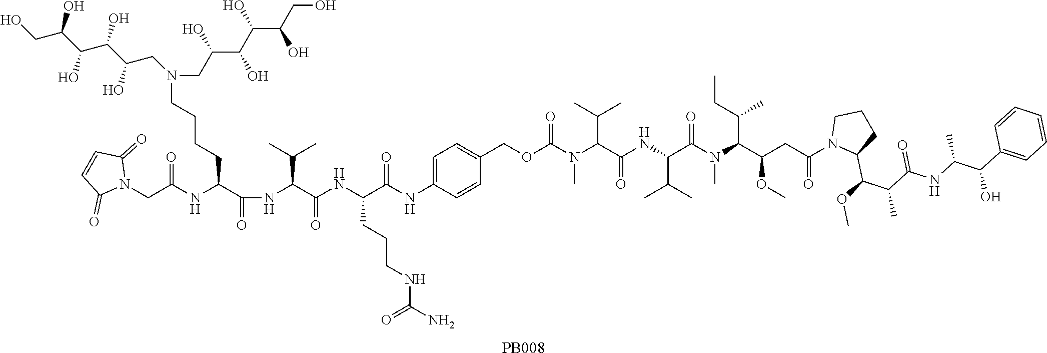

A Drug-Linker having one Sugar Unit and a cleavable linker attached to MMAE (PB008) was prepared as follows:

### Step 1

To a solution of compound 8-1 (155.50 mg, 0.203 mmol) in DMF (10 mL), HOBt (26.35 mg, 0.195 mmol) and MMAE (140 mg, 0.195 mmol) was added at 0° C. The reaction mixture was stirred at 0° C. for 15 min, Pyridine (3 mL) and DIEA ethyldiisopropylamine (0.039 mL, 0.234 mmol) were added at 0° C., and the reaction mixture was stirred at 0° C. for 30 min. The reaction mixture was allowed to warm to r.t. The reaction mixture was stirred at r.t. for 36 hr. After removing DIEA and pyridine, the residue was purified by Prep-HPLC to yield the desired product 8-2 (62 mg, 0.045 mmol). LCMS ((M+2H)/2)⁺=673.4;

### Step 2

To a solution of compound 8-2 (60 mg, 0.045 mmol) in DMF (0.95 mL) was added diethyl amine anhydrous (0.05 mL, 0.485 mmol). Then the resulting solution was stirred at room temperature for 1 h until LCMS indicated complete deprotection. The completed reaction solution was purified by reverse phase column chromatography (C18 column, eluting with 0-70% acetonitrile in water with 0.01% TFA) to get the desired fractions containing compound 8-3, which were lyophilized to yield a TFA salt of compound 8-3 (40 mg, 0.036 mmol, 79.85%) as a white solid. LCMS (M/2+H)⁺=562.5; 1H NMR (400 MHz, DMSO-d6) δ 10.20 (s, 1H), 8.69 (d, J=7.6 Hz, 1H), 8.30 (brs, 2H), 8.17-8.07 (m, 3H), 7.90 (d, J=8.4 Hz, 0.5H), 7.64 (d, J=8.4 Hz, 0.5H), 7.59-7.57 (m, 2H), 7.37-7.24 (m, 6H), 7.20-7.13 (m, 1H), 6.05-6.02 (m, 1H), 5.48 (s, 2H), 5.43-5.34 (m, 1H), 5.12-4.97 (m, 2H), 4.78-4.23 (m, 4H), 4.04-3.93 (m, 2H), 3.80-3.52 (m, 2H), 3.25-3.06 (m, 7H), 2.98-2.83 (m, 10H), 2.43-2.39 (m, 1H), 2.26-2.22 (m, 1H), 2.14-1.99 (m, 3H), 1.99-1.85 (m, 1H), 1.85-1.67 (m, 4H), 1.67-1.37 (m, 5H), 1.37-1.25 (m, 1H), 1.16 (t, J=7.2 Hz, 3H), 1.05-1.00 (m, 6H), 0.98-0.93 (m, 6H), 0.86-0.73 (m, 17H) ppm.

### Step 3

A solution of compound 8-3 (17.96 mg, 0.026 mmol), compound 8-4 and DIPEA (9.99 mg, 0.077 mmol) in anhydrous DMF (0.8 mL) was stirred at room temperature for 5 min, and then a solution of HATU (14.71 mg, 0.039 mmol) in anhydrous DMF (0.2 mL) was added. The resulting solution was stirred for another 1 hr at r.t. until LCMS indicated complete reaction. The reaction solution was purified directly by reverse phase liquid chromatography (C18 column, eluting with 0-70% acetonitrile in water with 0.01% TFA over 15 min) to yield compound 8-5 (36.2 mg, 0.020 mmol, 77.26%) as a white solid. LCMS (M/2+H)⁺=902.1; 1H NMR (400 MHz, DMSO-d6)

### Step 4

A solution of compound 8-5 (50 mg, 0.028 mmol) in DMF (0.95 mL) was stirred at r.t. and DEA (0.05 mL, 0.313 mmol) was added. The resulting solution was stirred at r.t. for 2 hr. The LCMS of the solution showed that the reaction was completed. The completed reaction solution was purified directly by reverse phase liquid chromatography (12 g C18 column, eluting with 0-60% acetonitrile in water with 0.01% TFA over 15 min) to give the expected fractions, which were lyophilized to yield product 8-6 (35 mg, 0.022 mmol, 79.85%) as a white solid. LCMS (M/2+H)⁺=790.6;

### Step 5

A solution of compound 8-6 (30 mg, 0.019 mmol), DIPEA (7.35 mg, 0.057 mmol) in anhydrous DMF (2 mL) was stirred at r.t., then compound 8-7 (2,5-dioxopyrrolidin-1-yl 2-(2,5-dioxo-2,5-dihydro-1H-pyrrol-1-yl)acetate) (9.58 mg, 0.038 mmol) was added. The resulting solution was stirred for another 1 hr to achieve complete conversion. The completed reaction solution was purified by Prep-HPLC Mobile Phase: A: Water (0.01% TFA) B: acetonitrile (0.01% TFA); Gradient Phase: 5% of B increasing to 95% of B with 15 min; Flow Rate: 1.0 mL/min; Column: SunFire C18, 4.6*50 mm, 3.5 μm; Column Temperature: 50° C. Detectors: ADC ELSD, DAD (214 nm and 254 nm) to afford TFA salt of PB008 (20 mg, 0.012 mmol, 61.34%) as a white solid. LCMS (M/2+H)⁺=859.0;

1H NMR (400 MHz, DMSO-d6) δ 10.03 (s, 1H), 8.42-8.40 (m, 2H), 8.33-8.31 (m, 0.5H), 8.20-8.18 (m, 1H), 8.11-8.09 (m, 0.5H), 7.92-7.84 (m, 1.5H), 7.66-7.63 (m, 0.5H), 7.59-7.56 (m, 2H), 7.36-7.24 (m, 6H), 7.20-7.14 (m, 1H), 7.10 (s, 2H), 6.06-6.01 (m, 1H), 5.54-5.33 (m, 4H), 5.13-4.95 (m, 2H), 4.87-4.34 (m, 10H), 4.30-4.17 (m, 2H), 4.09 (s, 2H), 4.04-3.92 (m, 4H), 3.80-3.77 (m, 0.5H), 3.70-3.65 (m, 2H), 3.61-3.58 (m, 9.5H), 3.33-3.24 (m, 2H), 3.23-3.07 (m, 13H), 3.03-2.89 (m, 5H), 2.89-2.83 (m, 3H), 2.44-2.39 (m, 1H), 2.30-2.21 (m, 1H), 2.16-2.05 (m, 2H), 1.99-1.91 (m, 2H), 1.84-1.22 (m, 18H), 1.06-0.97 (m, 6H), 0.89-0.75 (m, 26H) ppm. Proton signals of one molecule of TFA are included.

Drug linker PB008 can be used to make a conjugate such as PA008.

### Example 6: Preparation of the Drug-Linker (PB026) Having Two Sugar Units and a Cleavable Linker Attached to Exatecan

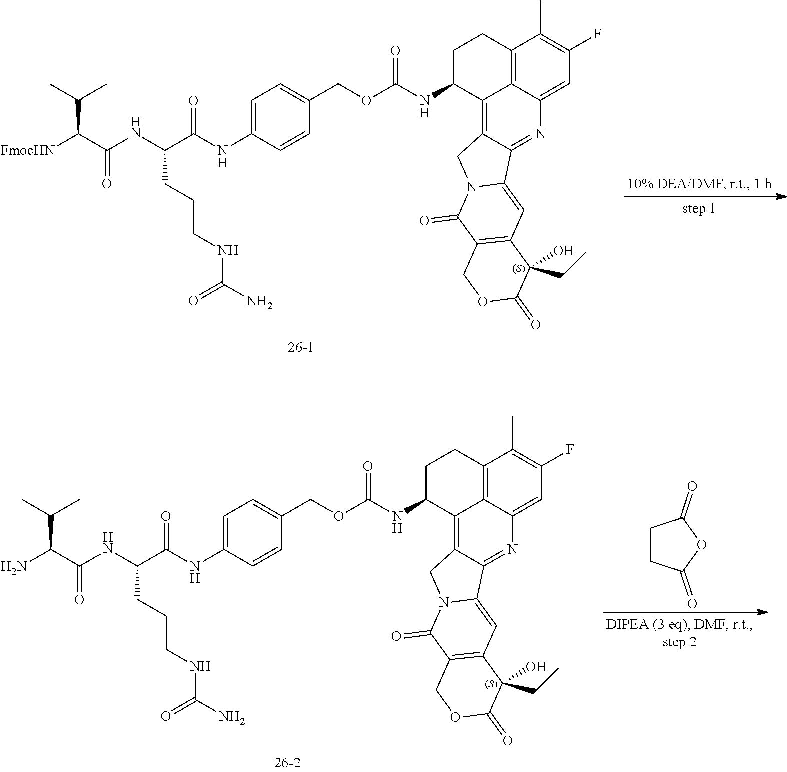

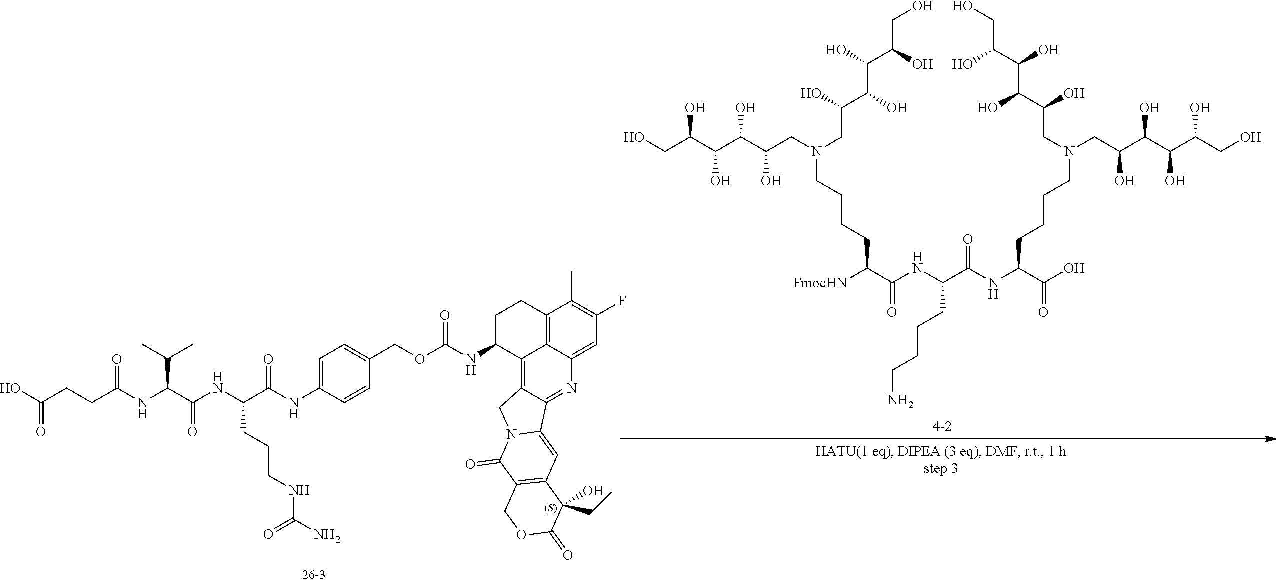

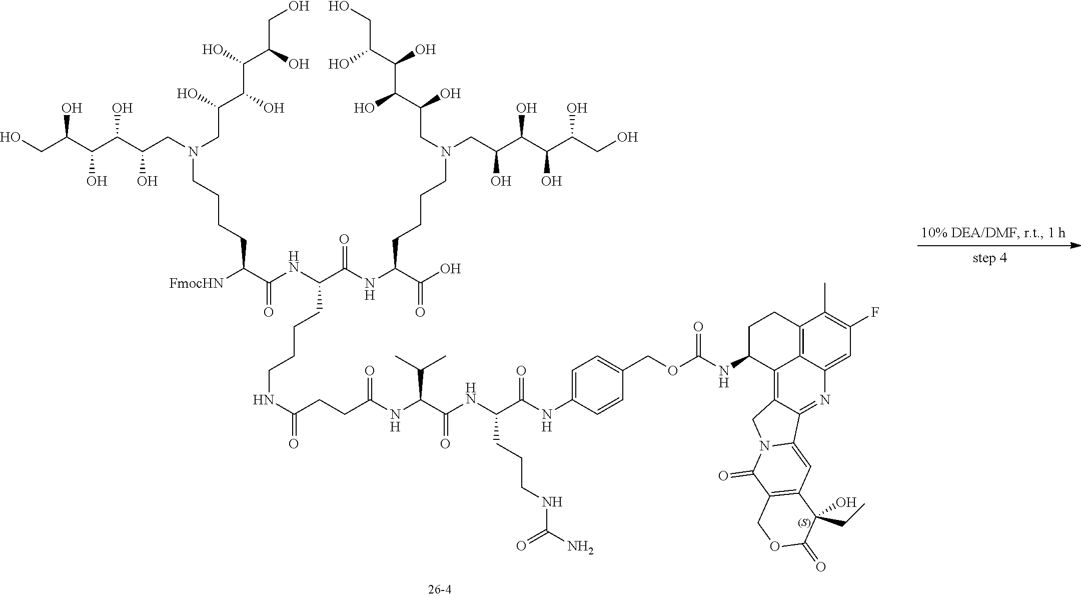

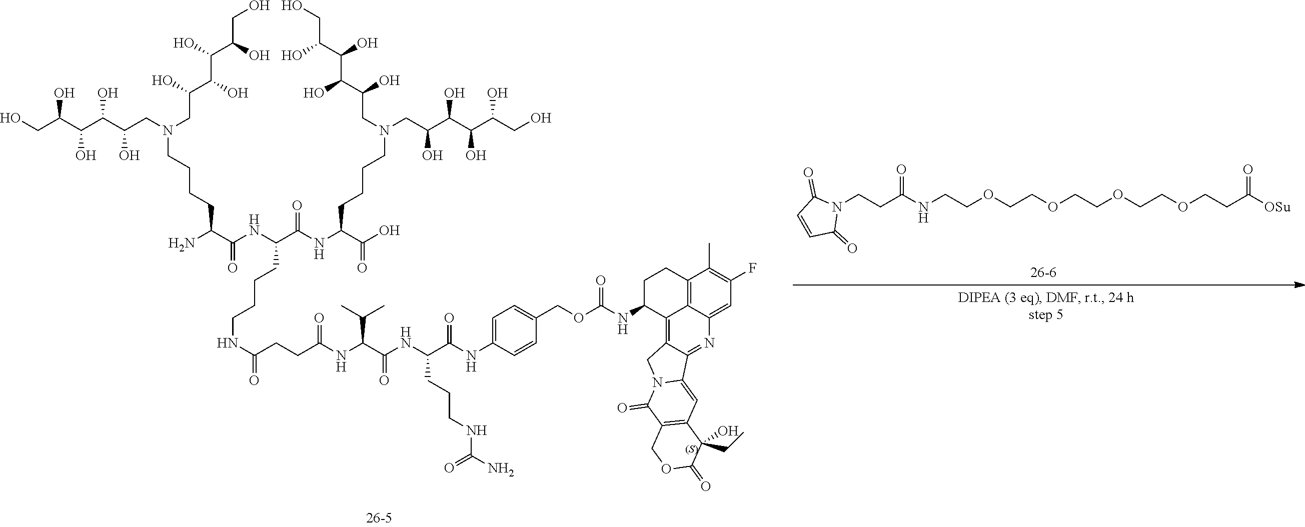

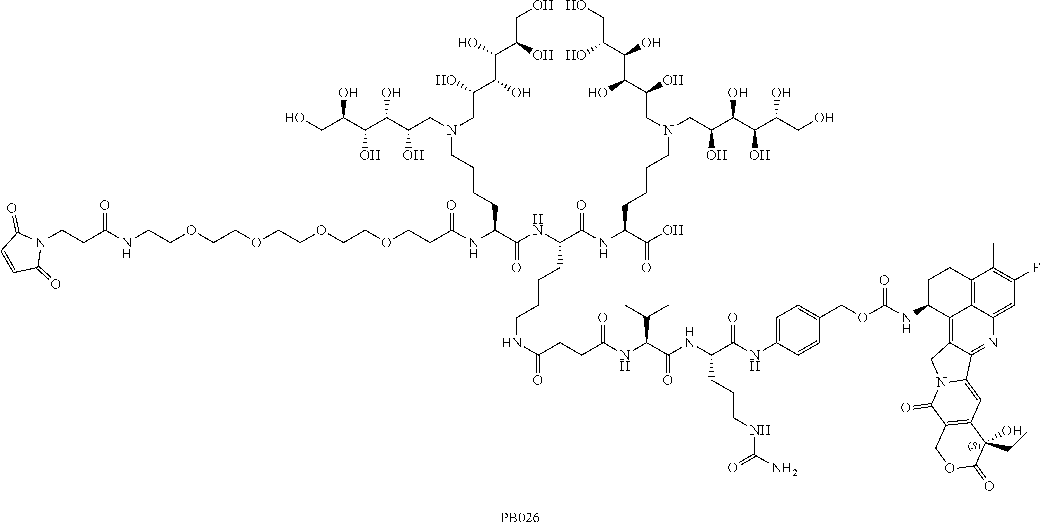

A Drug-Linker having two Sugar units and a cleavable linker attached to exatecan (PB026) was prepared as follows:

### Step 1

A solution of compound 26-1 (475 mg, 0.447 mmol) in DMF (3.6 mL) was stirred at r.t. and diethyl amine anhydrous (0.4 mL, 3.883 mmol) was added. The resulting solution was stirred at r.t. for 1 h. Then LCMS of the solution showed that the reaction was completed. The reaction solution was purified directly by reverse phase liquid chromatography (120 g C18 column, eluting with 0-80% acetonitrile in water with 0.01% TFA over 15 min) to yield product 26-2 (260 mg, 0.309 mmol, 69.24%) as a white solid. LCMS: m/z=841.1 (M+H)⁺

### Step 2

A solution of compound 26-2 (150 mg, 0.178 mmol) and oxolane-2,5-dione (35.70 mg, 0.357 mmol) in anhydrous DMF (2 mL) was stirred at room temperature, and then DIPEA (69.03 mg, 0.535 mmol) was added. The resulting solution was stirred at room temperature for 1 h until LCMS indicated all starting amine was consumed. The completed reaction solution was purified directly by reverse phase liquid chromatography (40 g C18 column, eluting with 0-70% acetonitrile in water with 0.01% TFA over 15 min) to yield compound 26-3 (140 mg, 0.149 mmol, 83.41%) as a white solid. LCMS: m/z=963.5 (M+Na)⁺

### Step 3

A solution of compound 26-3 (100 mg, 0.106 mmol), DIPEA (41.02 mg, 0.318 mmol) and HATU (40.30 mg, 0.106 mmol) in anhydrous DMF (1 mL) was stirred at room temperature for 10 min, and then a solution of compound 4-2 (271.57 mg, 0.212 mmol) in anhydrous DMF (1 mL) was added dropwise by syringe. After addition, the resulting solution was stirred for another 1 hr at r.t. until LCMS indicated the starting acid was almost consumed and the reaction was completed. The reaction solution was purified by reverse phase liquid chromatography (40 g C18 column, eluting with 0-70% acetonitrile in water with 0.01% TFA) to yield product 26-4 (100 mg, 0.045 mmol, 42.64%) as a white solid. LCMS ESI m/z=735.8 (M/2+H)⁺;

### Step 4

To a suspension of compound 26-4 (100 mg, 0.045 mmol) in acetonitrile (0.9 mL) was added water (0.9 mL) to help dissolve most of the material. Then diethyl amine anhydrous (0.2 mL, 1.941 mmol) was added to the solution and it was stirred at r.t. for 2 hours. During this process, a sticky oil precipitated on the bottom of the flask. The reaction was monitored by LCMS, the starting material was consumed and the desired product was detected as a major peak. The solution was concentrated to dryness and the residue was washed with petroleum ether to remove most of the nonpolar impurities. The undissolved solid was filtered and collected, then dissolved in acetonitrile and water (1:1) and lyophilized in a freeze dryer to yield compound 26-5 (60 mg, 0.030 mmol, 66.72%) as a white solid, which was used directly in the next step.

LCMS: ESI m/z=661.5 (M/3+H)⁺;

### Step 5

A mixture of compound 26-5 (30 mg, 0.015 mmol) and DIPEA (5.86 mg, 0.045 mmol) in anhydrous DMF (1 mL) was stirred at room temperature for 20 min, and solid was observed suspending in the solution, then additional DMF (1 mL) was added, followed by the addition of compound 26-6 (7.77 mg, 0.015 mmol). The resulting solution was stirred for at r.t. for 24 hr until LCMS and HPLC indicated starting amine was substantially consumed. The reaction solution was purified directly by Prep-HPLC (Mobile Phase: A: Water (0.01% FA) B: acetonitrile (0.01% FA); Gradient Phase: 5% of B increasing to 95% of B with 15 min; Flow Rate: 1.0 mL/min; Column: SunFire C18, 4.6*50 mm, 3.5 μm; Column Temperature: 50° C. Detectors: DAD (214 nm and 254 nm) to yield desired product PB026 (9.1 mg, 0.004 mmol, 25.26%) as a white solid. LCMS: ESI m/z=794.3 (M/3+H)⁺;

1H NMR (400 MHz, DMSO-d6) δ 9.73 (s, 1H), 8.38-8.31 (m, 1H), 8.20-8.16 (m, 2H), 8.08-8.00 (m, 4H), 7.89-7.82 (m, 1H), 7.78 (d, J=11.2 Hz, 1H), 7.65 (d, J=8.0 Hz, 2H), 7.41-7.35 (m, 2.6H), 7.31 (s, 1H), 7.01-7.00 (m, 1.4H), 6.52 (brs, 1H), 6.09-6.01 (m, 1H), 5.45 (s, 4H), 5.29 (s, 4H), 5.08 (s, 2H), 4.36-4.27 (m, 2H), 4.15-4.12 (m, 1H), 4.09-4.03 (m, 0.5H), 3.96-3.88 (m, 1H), 3.88-3.82 (m, 0.5H), 3.77-3.70 (m, 1H), 3.64-3.57 (m, 12H), 3.50-3.47 (m, 17H), 3.43-3.40 (m, 10H), 3.40-3.22 (m, 11H), 3.16-3.12 (m, 4H), 3.05-2.90 (m, 11H), 2.72-2.50 (m, 3H), 2.38-2.31 (m, 11H), 2.24-2.12 (m, 5H), 2.08-1.98 (m, 2H), 1.93-1.87 (m, 2H), 1.84-1.55 (m, 8H), 1.55-1.23 (m, 18H), 1.13-1.07 (m, 2H), 1.01-0.96 (m, 3H), 0.89-0.85 (m, 9H) ppm.

Drug linker PB026 can be used to make a conjugate such as PA026.

### Example 7: Preparation of the Drug Linker (PB037) Containing Two Sugar Units and a Cleavable Linker Attached to Exatecan

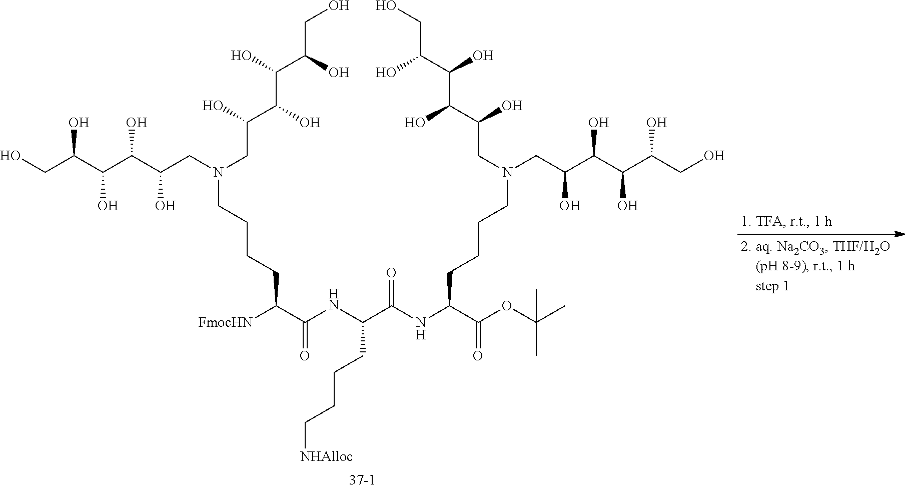

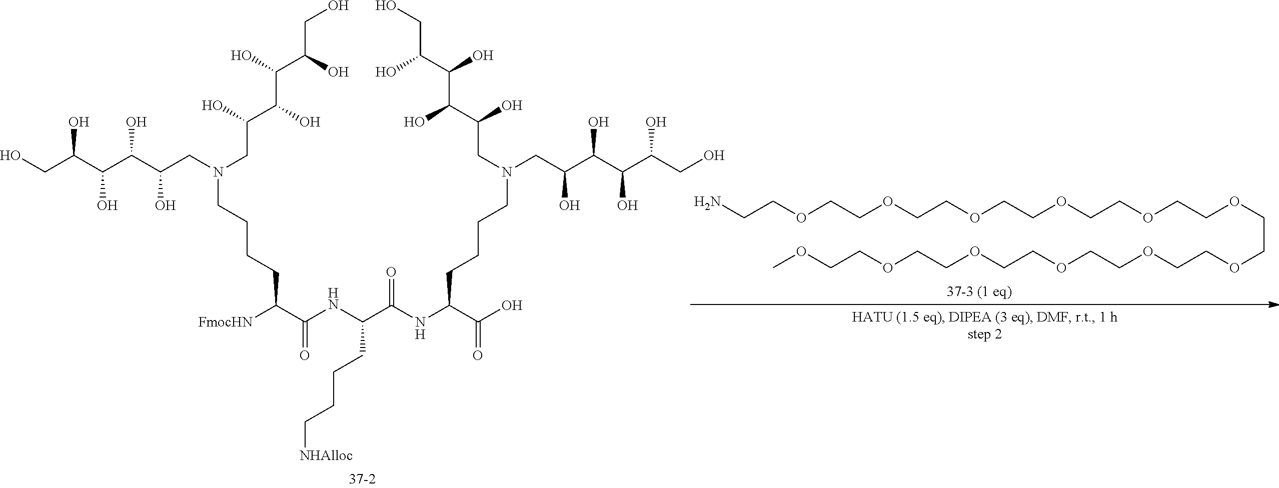

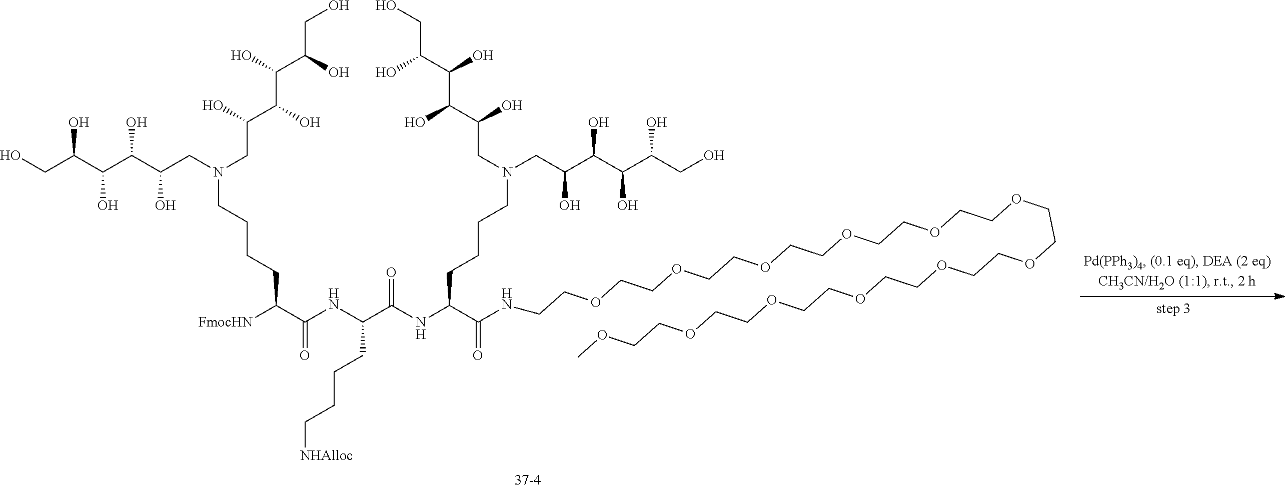

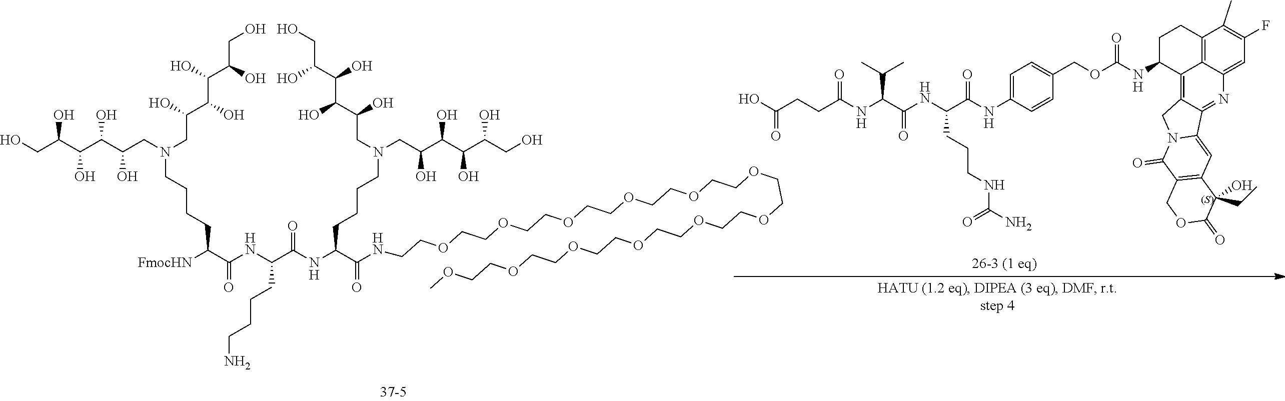

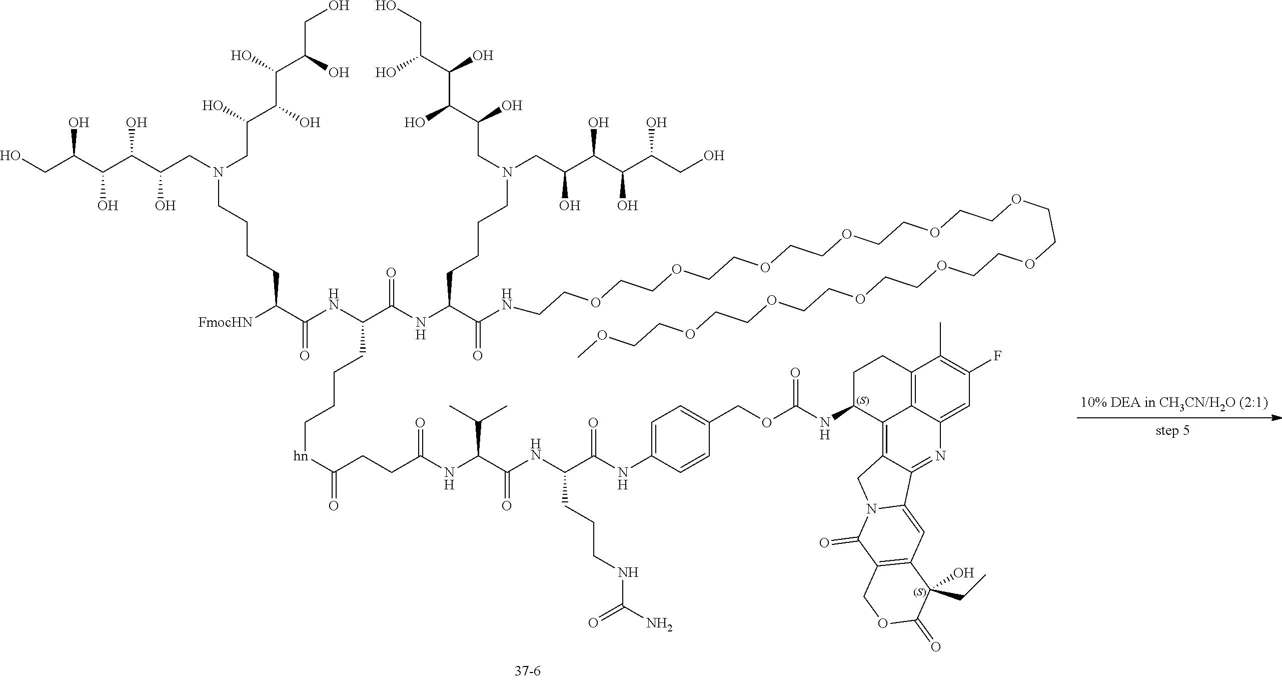

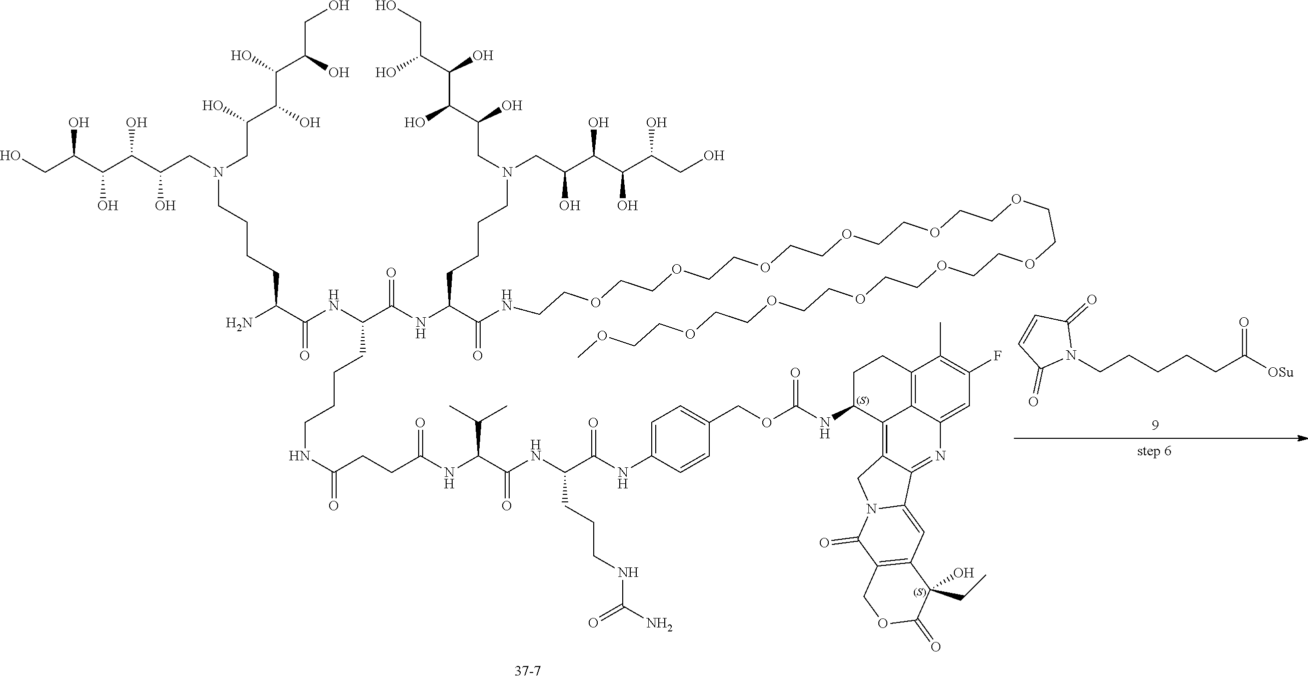

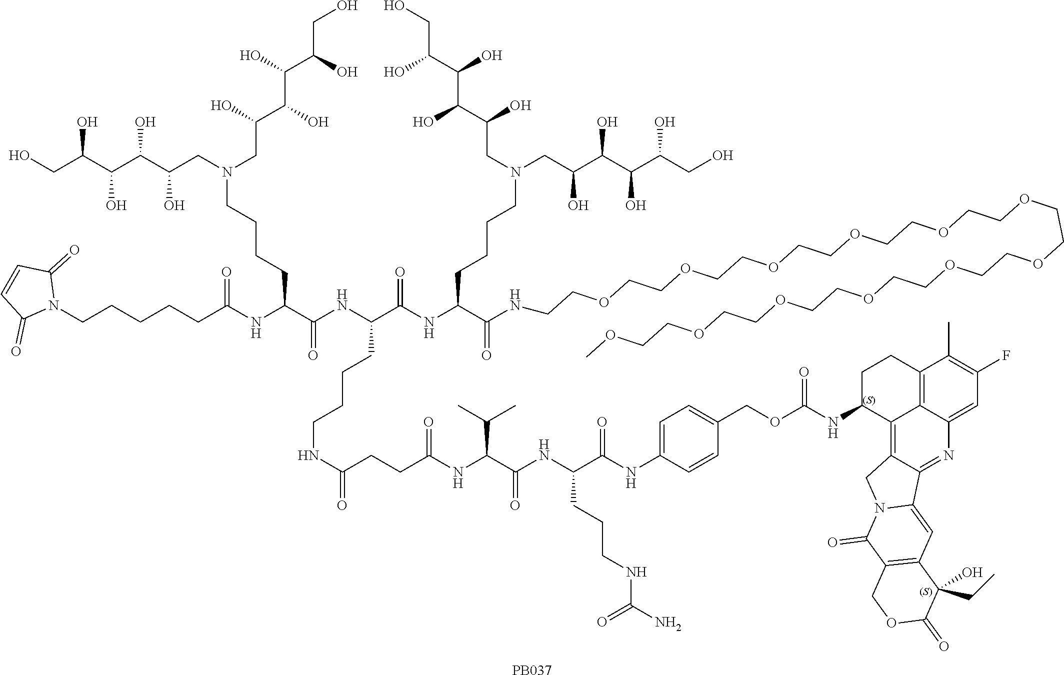

A Drug-Linker containing two Sugar units and a cleavable linker attached to exatecan (PB037) was prepared as follows:

### Step 1

A solution of compound 37-1 (200 mg, 0.141 mmol) in TFA (2 mL) was stirred at r.t. for 2 hr. LCMS of the mixture showed that the reaction was completed and all starting material was consumed and the desired product (mass 640=1280/2) was formed. The completed reaction solution was condensed to dryness and then redissolved in THF (4 mL) and water (1 mL), and treated with saturated aqueous sodium carbonate solution to adjust the pH to 8-9. The resulting solution was stirred at room temperature for 1 h to achieve completion. The solution was then neutralized with diluted TFA and condensed, and the residue was purified by reverse phase liquid chromatography (C18 column, eluting with 0-30% acetonitrile in water with 0.01% TFA for 15 min) to yield the expected product 37-2 (180 mg, 0.132 mmol, 93.70%) as a white solid after lyophilization. LCMS, ESI m/z=683.4 (M/2+H)⁺;

### Step 2

A solution of compound 37-2 (180 mg, 0.132 mmol), HATU (75.27 mg, 0.198 mmol) and DIPEA (51.07 mg, 0.396 mmol) in anhydrous DMF (2 mL) was stirred at room temperature for 5 min and compound 37-3 (73.86 mg, 0.132 mmol) was added. The resulting solution was stirred for another 1 hr at r.t. until LCMS indicated a complete reaction. The reaction solution was purified directly by reverse phase liquid chromatography (40 g C18 column, eluting with 0-70% acetonitrile in water with 0.01% TFA over 15 min) to yield compound 37-3 (220 mg, 0.118 mmol, 89.48%) as a white solid. LCMS, ESI m/z=954.5 (M/2+Na)⁺;

### Step 3

A solution of compound 37-3 (220 mg, 0.115 mmol) and Pd(PPh3)4 (133.30 mg, 0.115 mmol) in anhydrous acetonitrile (1 mL) and water (1 mL) was stirred at room temperature, and then diethyl amine anhydrous (0.024 mL, 0.231 mmol) was added immediately. The resulting solution was stirred for another 2 hr at r.t. until LCMS indicated all starting material was consumed and the mass of the desired product was detected. The resulting solution was concentrated under reduced pressure to remove solvent and diethyl amine. The residue was then purified directly by Prep-HPLC (eluting with gradient with 0.01% TFA over 20 min) to yield compound 37-4 (160 mg, 0.088 mmol, 76.08%) as a white solid. LCMS, m/z=608.6 (M/3+H), 912.2 (M/2+H)⁺;

### Step 4

A solution of compound 37-4 (30.97 mg, 0.033 mmol), HATU (12.51 mg, 0.033 mmol) and DIPEA (4.25 mg, 0.033 mmol) in anhydrous solvent was stirred at room temperature for 5 min, then compound 26-1 (60 mg, 0.033 mmol) was added. The resulting solution was stirred for another 2 hr at r.t. until LCMS indicated a complete reaction. The reaction solution was purified directly by reverse phase liquid chromatography (40 g C18 column, eluting with 0-70% acetonitrile in water with 0.01% TFA over 15 min) to give compound 37-5 (40 mg, 0.015 mmol, 44.26%) as a white solid. LCMS, ESI m/z=916.4 (M/3+H)⁺;

### Step 5

A solution of compound 37-5 (40 mg, 0.015 mmol) in CH₃CN (1.2 mL) and water (0.6 mL) was stirred at r.t., and then Diethyl Amine Anhydrous (0.2 mL, 0.015 mmol) was added. The resulting solution was stirred at r.t. for overnight until LCMS showed that the reaction was completed. Solvent and most of diethyl amine were evaporated, and then the residue was purified by reverse phase liquid chromatography (12 g C18 column, eluting with acetonitrile in water with 0.01% TFA) to yield the expected product 37-6 (20 mg, 0.008 mmol, 54.41%) as a white solid. LCMS, ESI m/z=631.8 (M/4+H)⁺, 842.1 (M/3+H)⁺; 2524

### Step 6

A solution of compound 37-6 (20 mg, 0.008 mmol) and DIPEA (5.03 mg, 0.039 mmol) in anhydrous DMF (0.4 mL) was stirred at room temperature for 5 min, and then a solution of 2,5-dioxopyrrolidin-1-yl 6-(2,5-dioxo-2,5-dihydro-1H-pyrrol-1-yl)hexanoate (5.83 mg, 0.019 mmol) in anhydrous DMF (0.1 mL) was added dropwise by syringe over 2 min. The resulting solution was stirred for another 8 hr at r.t. until LCMS indicated all starting amine was consumed and the mass of the desired product was detected. The reaction solution was adjusted to pH 6-7 with diluted formic acid in acetonitrile, and then purified directly by Prep-HPLC (eluting with gradient with 0.01% FA over 20 min) to yield PB037 (4.8 mg, 0.002 mmol) as a white solid. LCMS, m/z=725.8 (M/3+H)⁺; Drug linker PB037 can be used to make a conjugate such as PA037.

### Example 8: Preparation of the Drug-Linker (PB038 or “LD038”) Containing a PEG Unit and a Cleavable Linker Attached to Exatecan

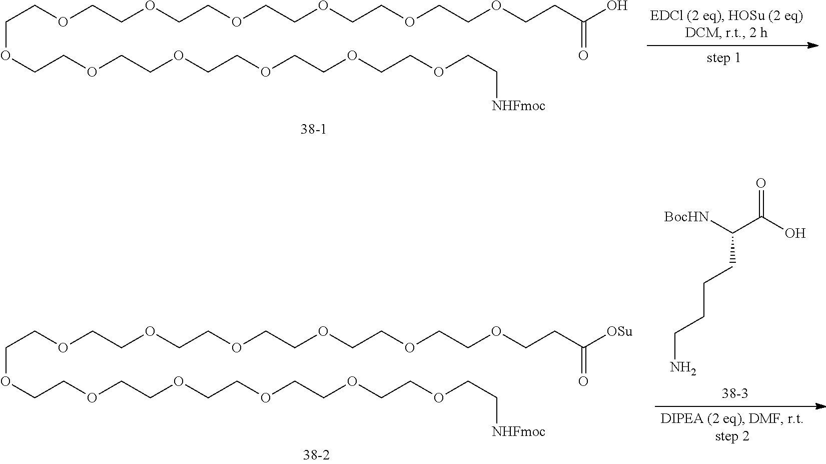

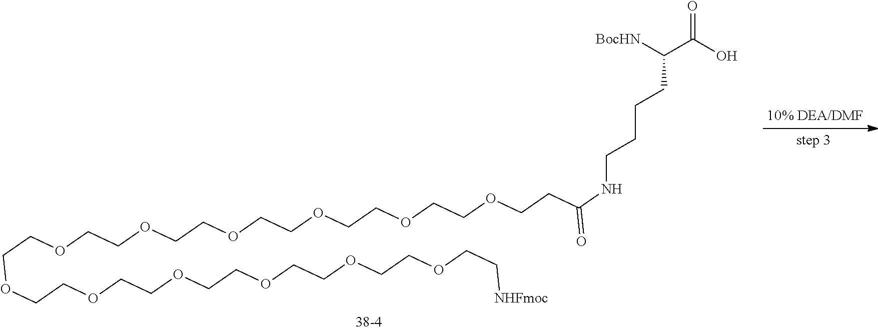

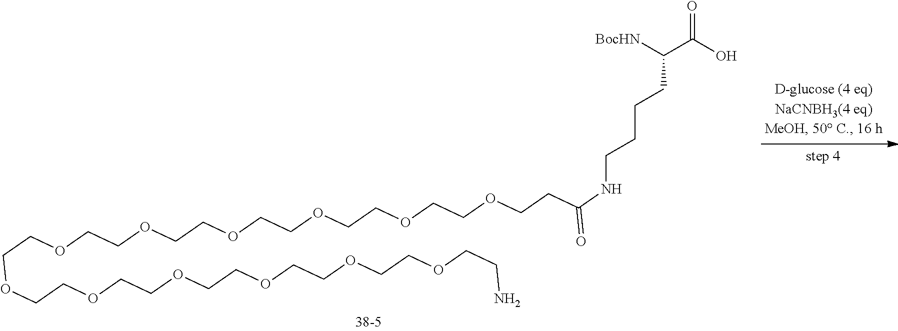 [SMILES: CC(C)(C)OC(=O)N[C@@H](CCCCNC(=O)CCOCCOCCOCCOCCOCCOCCOCCOCCOCCOCCOCCOCCN)C(=O)O]

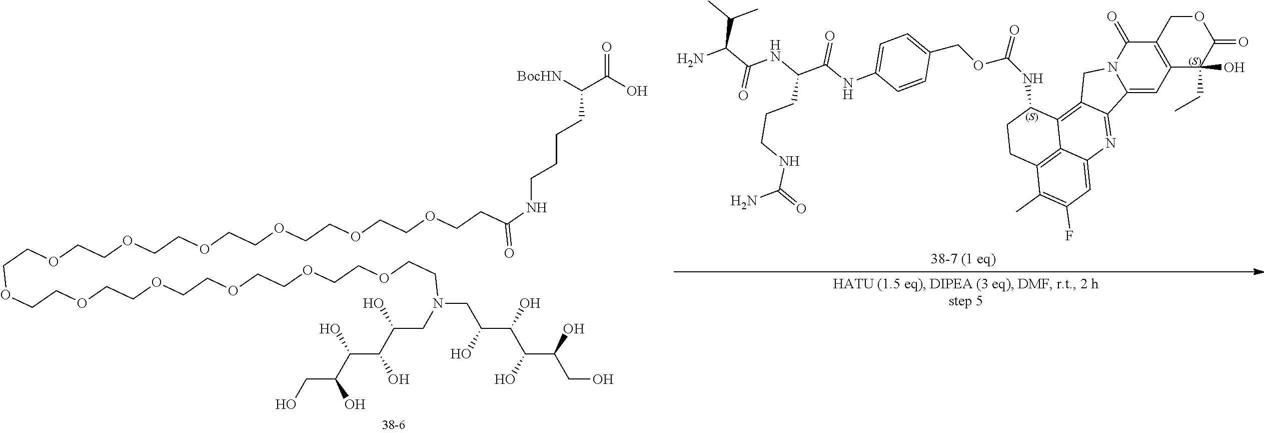

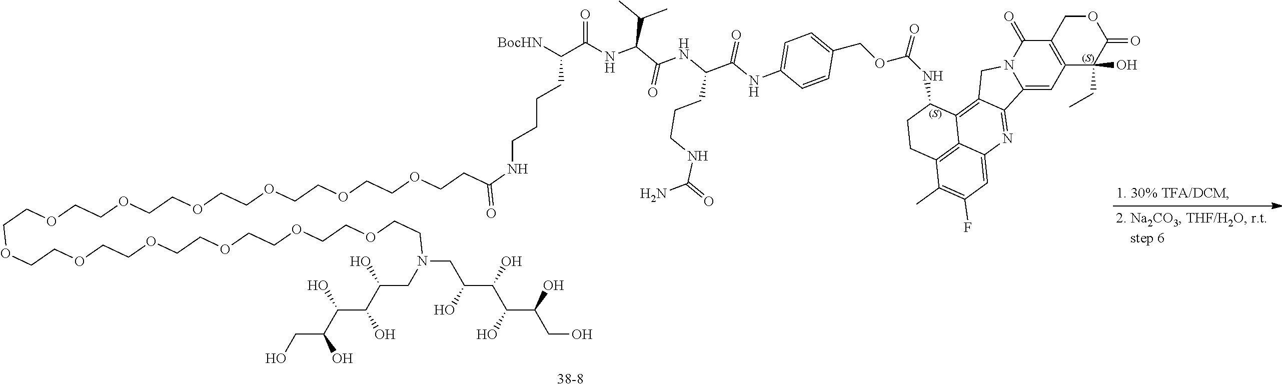

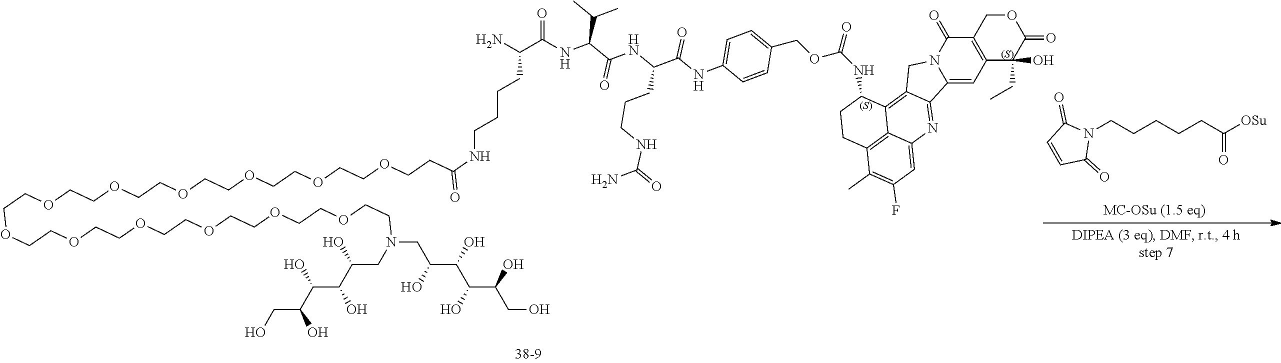

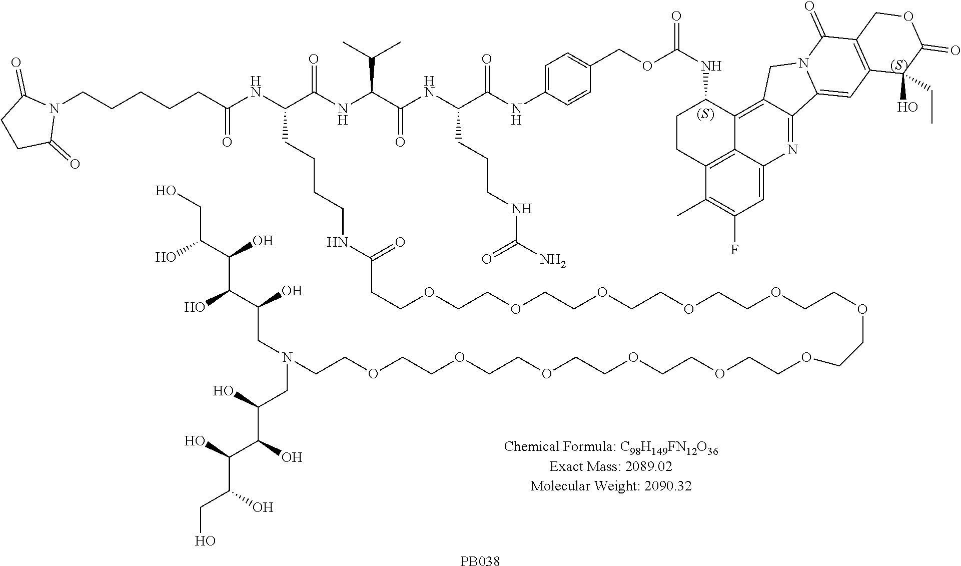

A Drug-Linker (PB038 or LD038) containing a PEG unit and a cleavable linker attached to exatecan was prepared as follows:

### Step 1

A solution of compound 38-1 (650 mg, 0.774 mmol) and N-hydroxylsuccinimide (HOSu) (177.98 mg, 1.548 mmol) in anhydrous DCM (8 mL) was stirred at room temperature, and then EDCI (296.69 mg, 1.548 mmol) was added. The resulting solution was stirred for another 1 hr at r.t. until LCMS indicated that all starting amine was consumed and the desired product was detected. The resulting solution was washed with water, the organic layer was collected, and then the water phase was extracted with DCM (10 mL*2). The combined organic layer was dried over sodium sulfate and filtered, concentrated to dryness to yield compound 38-2 (552 mg, 0.589 mmol, 76.12%) as colorless oil and used as such in the next step (refer to N200897-136). LCMS: m/z=959.4 (M+Na)⁺;

### Step 2

A solution of compound 38-2 (300 mg, 0.357 mmol) and DIPEA (138.22 mg, 1.071 mmol) in anhydrous DMF (2 mL) was stirred at room temperature, and then compound 38-3 (87.97 mg, 0.357 mmol) was added and the starting amine was suspended in the solution. The resulting mixture was kept stirring at r.t. for another 6 hrs. The starting amine dissolved gradually during this period, and the suspension turned into clear light yellow solution. The reaction solution was terminated and purified directly by reverse phase liquid chromatography (40 g C18 column, eluting with 0-100% acetonitrile in water with 0.01% TFA over 15 min) to yield compound 38-4 (260 mg, 0.243 mmol, 68.14%) as pale yellow oil, LCMS ((M−100)/2+H)⁺=484.9;

### Step 3

A solution of compound 38-4 (260 mg, 0.243 mmol) in acetonitrile (1.8 mL) was stirred at r.t. and diethyl amine anhydrous (0.2 mL, 1.941 mmol) was added. The resulting solution was stirred at r.t. for 2 h until the LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to yield the expected fractions of compound 38-5 (170 mg, 0.201 mmol, 82.54%) as pale yellow oil. LCMS, ESI m/z=846.6 (M+H)⁺; Retention time (0.01% TFA)=1.451 min; no UV.

### Step 4

A clear reaction solution of 38-5 (170 mg, 0.201 mmol), D-glucose (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and D-glucose (217.08 mg, 1.206 mmol) were added and kept stirring at 50° C. for overnight. After stirring for 20 hr, LCMS indicated the reaction was complete. The solvents were evaporated, and the residue was purified by C18 reversed-phase chromatography to yield the desired product 38-6 (106 mg, 0.090 mmol, 44.92%). LCMS, ESI m/z=537.9 ((M−100)/2+H)⁺;

### Step 5

A solution compound 38-6 (250 mg, 0.213 mmol), HATU (121.45 mg, 0.319 mmol) and DIPEA (82.41 mg, 0.639 mmol) in anhydrous DMF (2 mL) was stirred at room temperature for 5 min, and then compound 38-7 (178.88 mg, 0.213 mmol) was added. The resulting solution was stirred for another 2 hr at r.t. until LCMS indicated a complete reaction. The reaction solution was purified directly by reverse phase liquid chromatography (40 g C18 column, eluting with 0-70% acetonitrile in water with 0.01% TFA over 15 min) to yield compound 38-8 (270 mg, 0.135 mmol, 63.48%) as a white solid. LCMS, ESI m/z=666.6 (M/3+H)⁺, 999.2 (M/2+H)⁺;

### Step 6

A solution of compound 38-8 (120 mg, 0.060 mmol) in TFA (2 mL) was stirred at r.t. for 1 hr. The LCMS of the mixture showed that the reaction was completed, all starting material was consumed, and the desired product (m/z=633=1896/3+H, R.T. 1.501 min) along with the sugar-esterification product (TFA was condensed with hydroxy group in sugar unit, mono-ester with m/z=(1896+96)/2+H=665, R.T. 1.58 min) were formed. The completed reaction solution was condensed to dryness and then redissolved in THF (4 mL) and water (2 mL), and treated with saturated aqueous sodium carbonate solution to adjust the pH to 8-9. The resulting solution was stirred at room temperature for 1 h to achieve complete hydrolysis. The solution was then neutralized with diluted TFA and condensed. The residue was purified by reverse phase liquid chromatography (C18 column, eluting with 0-25% acetonitrile in water with 0.01% TFA for 15 min) to yield the expected product 38-9 (80 mg, 0.042 mmol, 70.19%) as a white solid after lyophilization. LCMS, ESI m/z=633.2 (M/3+H)⁺, 949.2 (M/2+H);

### Step 7

A solution of compound 38-9 (20 mg, 0.011 mmol) and DIPEA (4.08 mg, 0.032 mmol) in anhydrous DMF (1 mL) was stirred at room temperature for 5 min, and then a solution compound 38-10 (4.88 mg, 0.016 mmol) in anhydrous DMF (1 mL) was added dropwise by syringe over 2 min. The resulting solution was stirred for another 4 hr at r.t. until all starting amine was consumed and the mass of the desired product was detected. The resulting solution was neutralized with formic acid to adjust the pH to 6-7. Then the reaction solution was purified by Prep-HPLC (eluting with gradient with 0.01% TFA over 20 min) to yield PB038 (11 mg, 0.005 mmol, 49.91%) as a white solid. LCMS, m/z=697.7 (M/3+H)⁺;

¹HNMR (400 MHz, DMSO-d6): δ 10.03 (s, 1H), 8.19-8.11 (m, 2H), 8.07 (d, J=8.8 Hz, 1H), 7.96 (d, J=7.6 Hz, 1H), 7.82-7.77 (m, 2H), 7.66 (d, J=8.4 Hz, 1H), 7.60 (d, J=8.4 Hz, 2H), 7.36 (d, J=8.0 Hz, 2H), 7.32 (s, 1H), 7.00 (s, 2H), 6.53 (s, 1H), 5.99 (t, J=5.6 Hz, 1H), 5.45-5.43 (m, 6H), 5.30-5.24 (m, 3H), 5.08 (s, 2H), 4.84-4.74 (m, 2H), 4.65-4.49 (m, 4H), 4.45-4.35 (m, 3H), 4.27-4.17 (m, 2H), 4.04-3.95 (m, 2H), 3.80-3.77 (m, 2H), 3.71-3.67 (m, 2H), 3.62-3.55 (m, 9H), 3.53-3.43 (m, 44H), 3.27-3.21 (m, 2H), 3.16-3.07 (m, 2H), 3.07-2.93 (m, 6H), 2.38 (s, 3H), 2.29 (t, J=6.4 Hz, 2H), 2.23-2.13 (m, 2H), 2.13-2.08 (m, 2H), 2.00-1.82 (m, 4H), 1.73-1.54 (m, 4H), 1.54-1.40 (m, 7H), 1.40-1.30 (m, 4H), 1.30-1.14 (m, 5H), 0.90-0.81 (m, 9H) ppm.

Drug linker PB038 can be used to make a conjugate such as PA038.

### Example 9: Preparation of the Drug-Linker (PB039) Containing a PEG Unit Attached to a Cleavable Linker and Exatecan

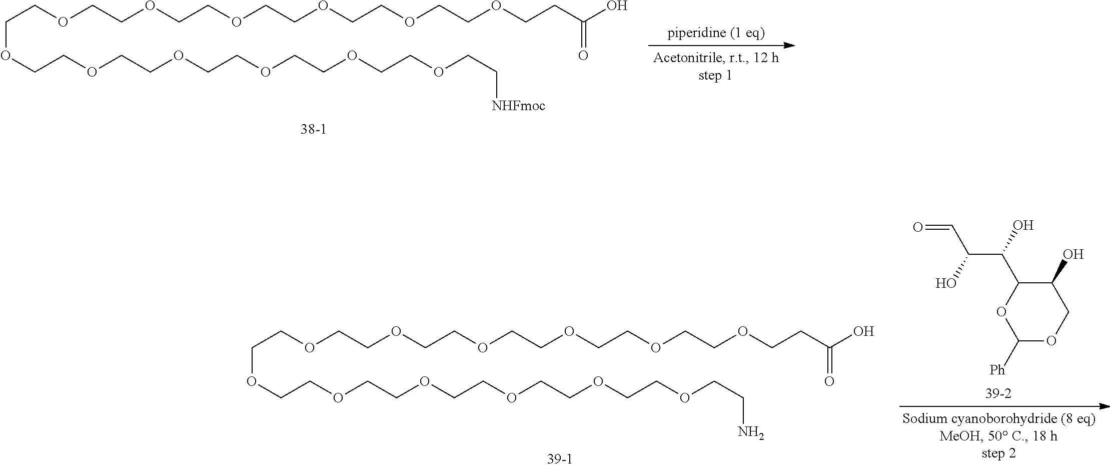

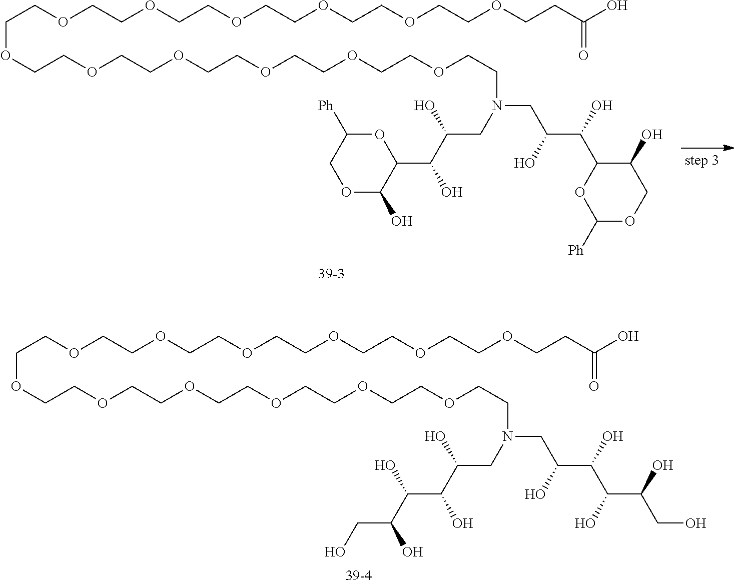 [SMILES: O=C(O)CCOCCOCCOCCOCCOCCOCCOCCOCCOCCOCCOCCOCCN(C[C@@H](O)[C@H](O)C1OC(c2ccccc2)OC[C@@H]1O)C[C@@H](O)[C@H](O)C1OC(c2ccccc2)CO[C@@H]1O ; O=C(O)CCOCCOCCOCCOCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO]

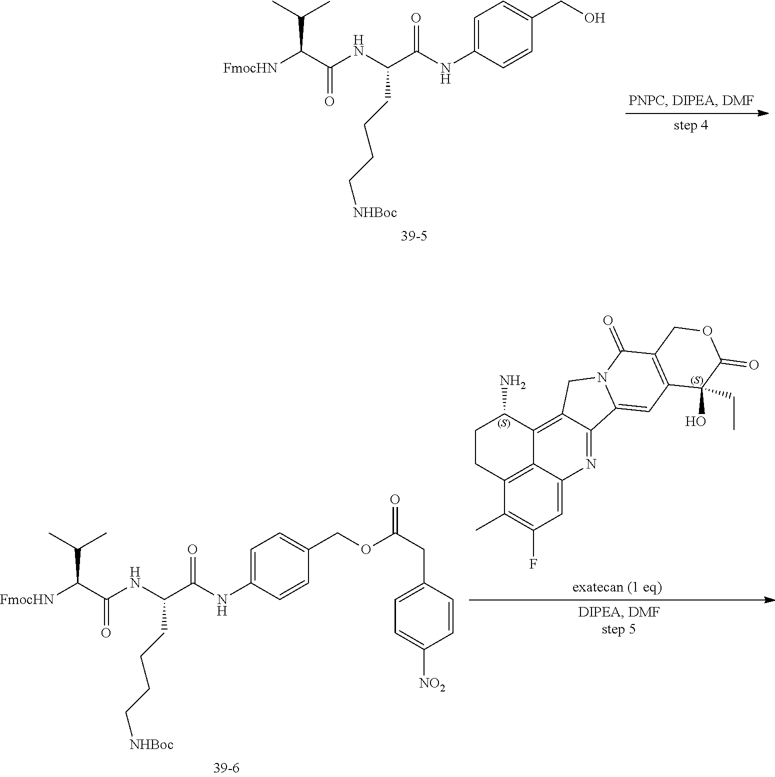

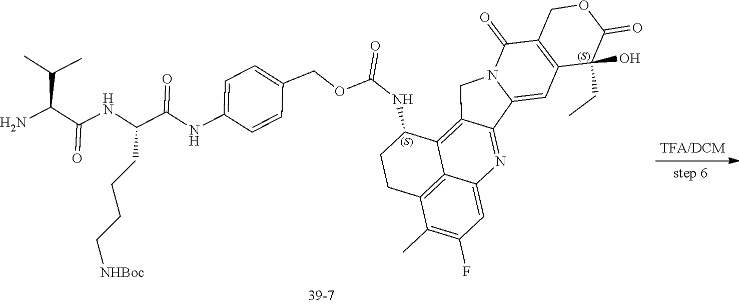

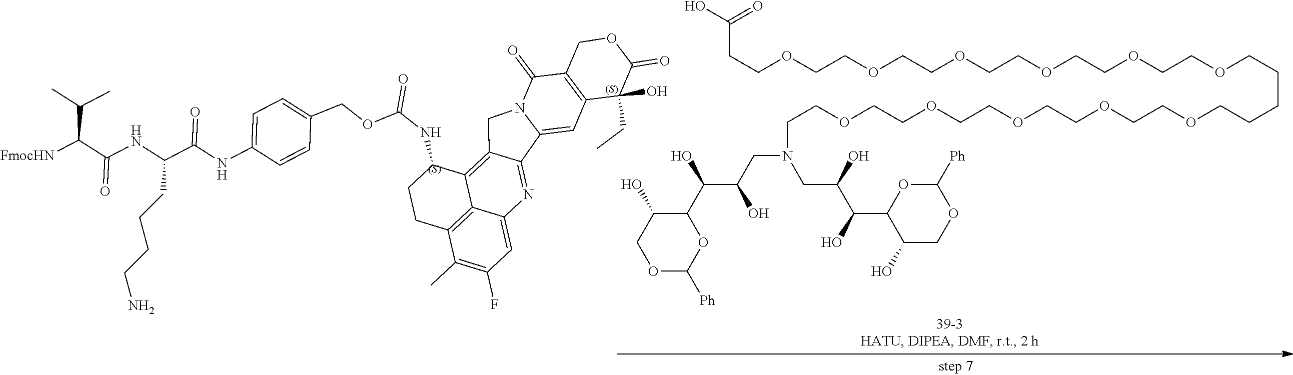

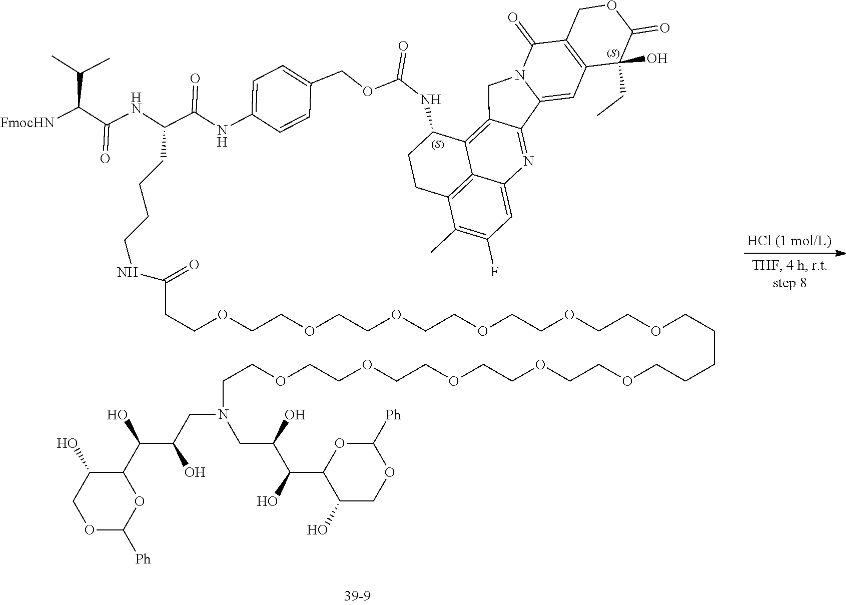

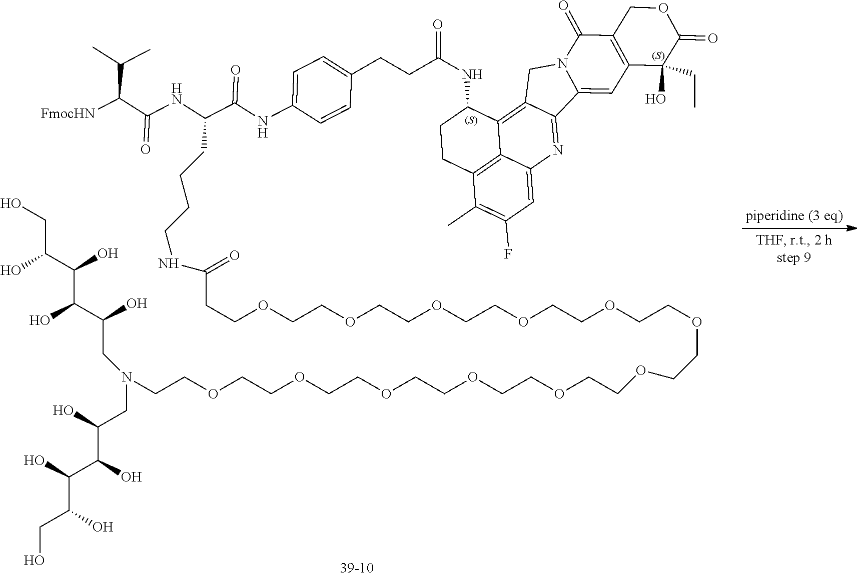

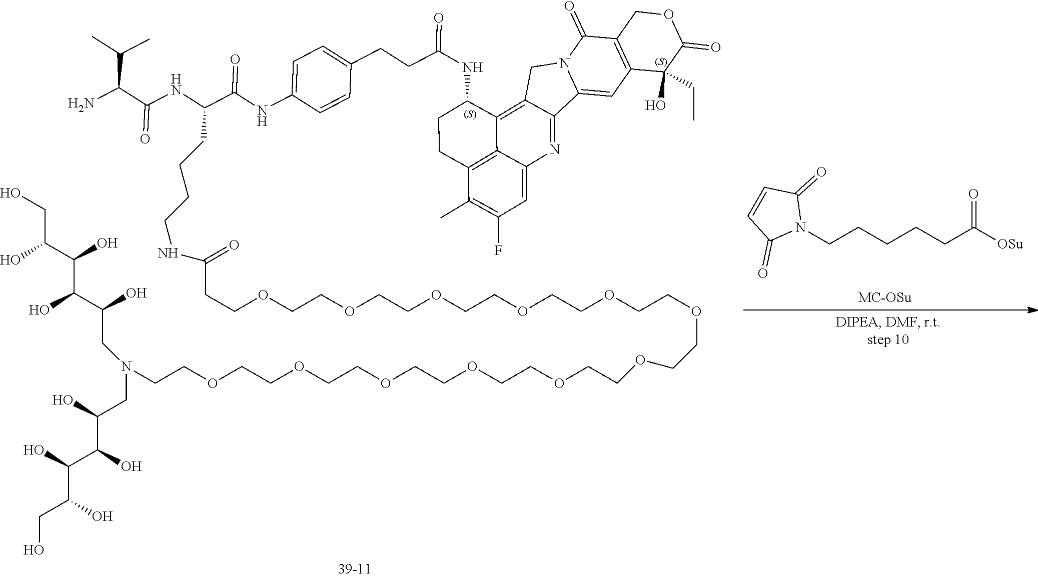

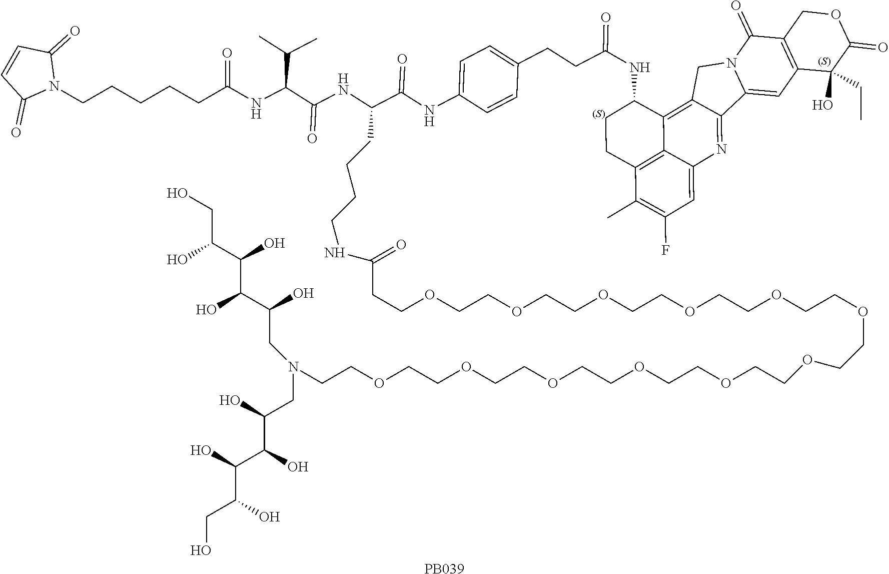

A Drug-Linker containing a PEG unit attached to a cleavable linker and exatecan (PB0039) was prepared as follows:

### Step 1

A solution of compound 38-1 (260 mg, 0.31 mmol) in acetonitrile (3.0 mL) was stirred at r.t. and diethyl amine anhydrous (0.2 mL, 1.941 mmol) was added. The resulting solution was stirred at r.t. for 2 h until LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to yield the expected fractions of compound 39-1 (170 mg, 0.28 mmol) as a pale yellow oil. LCMS, ESI m/z=618.4 (M+H)⁺;

### Step 2

A clear reaction solution of compound 39-1 (170 mg, 0.28 mmol), 39-2 (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and compound 39-2 (217.08 mg, 1.206 mmol) was added and kept stirring at 50° C. for overnight. After stirring for 20 hrs, LCMS indicated the reaction was complete. The solvents were evaporated, and the residue was purified by C18 reversed-phase chromatography to yield the desired product 39-3 (265 mg, 0.24 mmol). LCMS, ESI m/z=1122.6 (M+H)⁺.

### Step 3

A mixture of compound 39-3 (265 mg, 0.24 mmol) in 6N HCl/THF aqueous was stirred at rt for 3 hours, until LCMS indicated the reaction was complete. The solvents were neutralized with aqueous NaHCO₃, evaporated, and the residue was purified by C18 reversed-phase chromatography to yield the desired product 39-4 (160 mg, 0.17 mmol). LCMS, ESI m/z=946.5 (M+H)⁺.

### Step 4

A solution of compound 39-5 (3.3 g, 4.905 mmol) and DIPEA (1.90 g, 14.714 mmol) in anhydrous DMF (10 mL) was stirred at room temperature for 5 min, and then PNPC (4.47 g, 14.714 mmol) was added. The resulting bright yellow solution was stirred for another 1.5 hr at r.t. to achieve completion. The resulting solution was quenched with water and the solution was purified directly by reverse phase column chromatography (eluting with 0-100% acetonitrile in water) to yield compound 39-6 (1950 mg, 2.327 mmol) as a yellow solid. LCMS, m/z=860.4 (M+Na)⁺, 738.4 (M−100+H)⁺;

### Step 5

To a solution of compound 39-6 (1.2 g, 1.432 mmol) in DMF (12 mL) was added DIPEA (0.56 g, 4.296 mmol), HOBt (0.10 g, 0.716 mmol) and exatecan (0.69 g, 1.575 mmol). The mixture was stirred at room temperature for 3 h. The resulting solution was purified by reverse phase separation (C18 column, eluting with 0-100% methanol in water with TFA) to afford compound 39-7 (1.2 g, 1.058 mmol), ESI, m/z: 1135.5 (M+H)⁺

### Step 6

To the solution of compound 39-7 (620 mg, 0.547 mmol) in DCM (20 mL) was added TFA (2 mL, 0.555 mmol). The mixture was stirred at room temperature for 2 h. The resulting solution was concentrated and purified by reverse phase separation (C18 column, eluting with 0-50% acetonitrile in water with TFA) to afford the product compound 39-8 (409 mg, 0.395 mmol). ESI, m/z: 517.9 (M/2+H)⁺.

### Step 7

To a solution of compound 39-3 (600 mg, 0.254 mmol) in DMF (5 mL) was added HATU (97.65 mg, 0.254 mmol) and DIPEA (66.26 mg, 0.502 mmol). The reaction mixture was stirred at r.t. for 10 min. Then the mixture was combined with compound 39-8 (550 mg, 0.232 mmol). The reaction mixture was stirred at r.t. for 2 hours. The resulting solution was concentrated and purified by reverse phase separation (C18 column, eluting with 0-50% acetonitrile in water with TFA) to afford the product 39-9 (500 mg) as a white solid. ESI, m/z: 713.5 (M/2+H)⁺.

### Step 8

To a solution of compound 39-9 (150 mg, 0.070 mmol) in THF (2 mL) was added HCl (2 mol/L, 2 mL). The reaction mixture was stirred at r.t. for 3 hours. The resulting solution was concentrated and purified by reverse phase separation (C18 column, eluting with 0-50% acetonitrile in water with TFA) to afford the product 39-10 (100 mg, 51 mmol) as a white solid. yield 65.69%, purity=90%. ESI, m/z: 655.1 (M/3+H)⁺.

### Step 9

To a solution of compound 39-10 (350 mg, 0.178 mmol) in DMF (2 mL) was added piperidine (76 mg, 0.896 mmol). Then the mixture was stirred at r.t. for 2 hours. The resulting solution was concentrated and purified by reverse phase separation (C18 column, eluting with 0-50% acetonitrile in water with TFA) to afford the product 39-11 (160 mg, 0.092 mmol) as a white solid, yield=51.55%. ESI, m/z: 871.0 (M/2+H)⁺.

### Step 10

To a solution of compound 39-11 (160 mg, 0.092 mmol) in DMF (2 mL) was added DIEA (24 mg, 0.184 mmol) and MC-OSu (56.64 mg, 0.184 mmol). The reaction mixture was stirred at room temperature for 4 hours. The resulting solution was concentrated and purified by reverse phase separation (C18 column, eluting with 0-50% acetonitrile in water with TFA) to afford the product PB039 (62 mg) as a white solid. yield=34.88%. LCMS, m/z=967.2 (M/2+H)⁺; 1HNMR (400 MHz, DMSO-d6): δ 9.96 (s, 1H), 8.04-8.07 (m, 2H), 7.75-7.83 (m, 3H), 7.3 (m, 3H), 6.99 (s, 1H), 6.54 (m, 2H), 5.66 (s, 1H), 5.08-5.28 (m, 3H), 4.51 (m, 2H), 4.14-4.50 (m, 9H), 3.45-3.67 (m, 64H), 1.75-2.75 (m, 19H), 1.73-1.30 (m, 12H), 1.40-1.30 (m, 4H), 1.30-1.14 (m, 5H), 0.90-0.81 (m, 9H) ppm.

Drug linker PB039 can be used to make a conjugate such as PA039.

### Example 10: Preparation of the Drug-Linker (PB040) Containing EDTA Attached to a Cleavable Linker and Exatecan

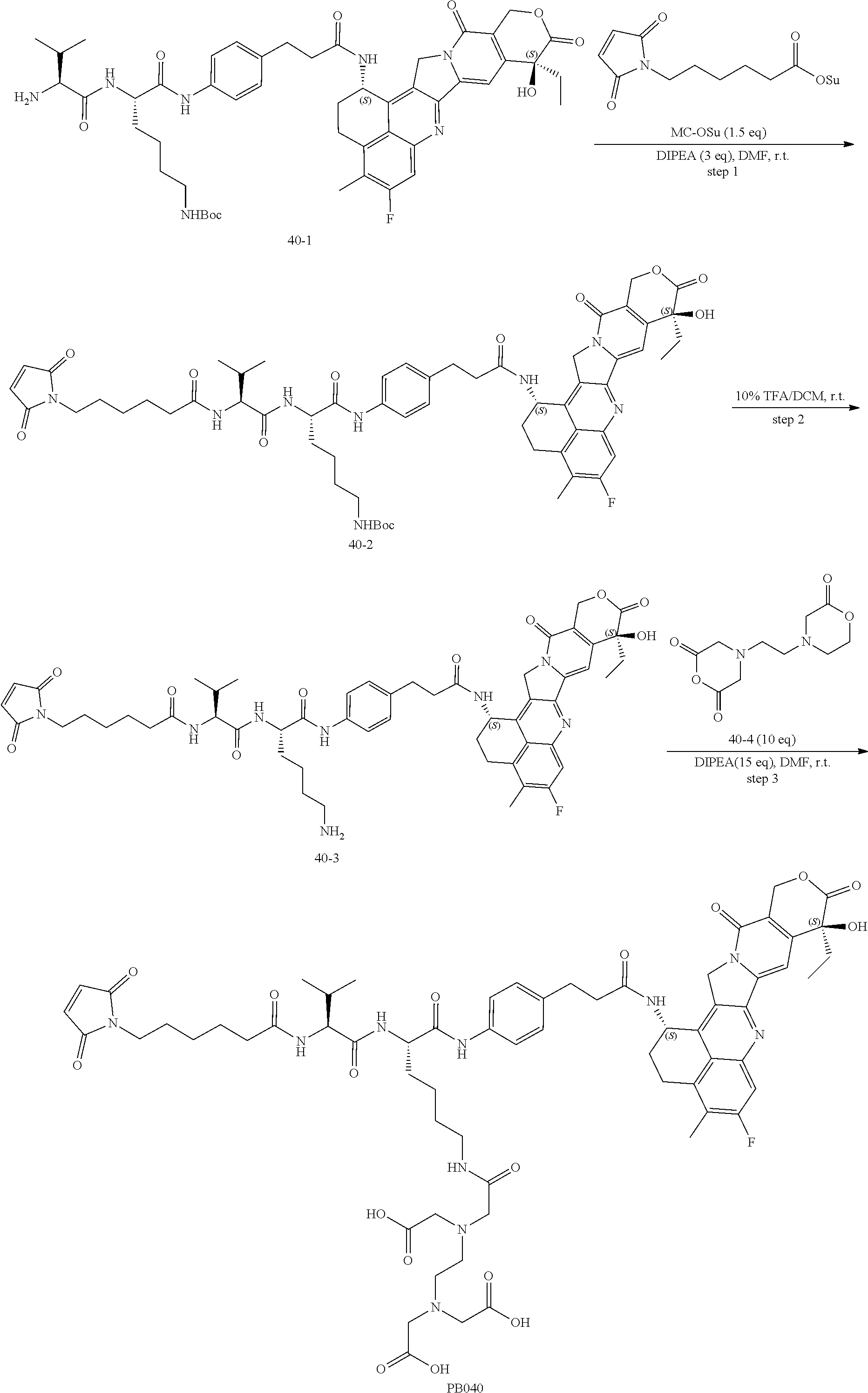

A Drug-Linker containing EDTA attached to a lysine residue of a cleavable linker was prepared as follows:

### Step 1

To a solution of compound 40-1 (31 mg, 0.034 mmol) in DMF (5 mL) was added DIPEA (13.18 mg, 0.102 mmol) and 2,5-dioxopyrrolidin-1-yl 6-(2,5-dioxo-2,5-dihydro-1H-pyrrol-1-yl)hexanoate (20.96 mg, 0.068 mmol). The mixture was stirred at room temperature for 2 h. The resulting solution was adjusted to pH 6 and purified by reverse phase separation (C18 column, eluting with 0-60% acetonitrile in water with TFA) to afford the product 40-2 (25.4 mg, 0.023 mmol, 67.61%), m/z: 1106.4 (M+H)⁺.

### Step 2

To a solution of compound 40-2 (83 mg, 0.075 mmol) in DCM (7 mL) was added TFA (0.5 mL, 0.031 mmol). The mixture was stirred at room temperature for 1 h. The resulting solution was concentrated and purified by reverse phase separation (C18 column, eluting with 0-30% acetonitrile in water with TFA) to afford the compound 40-3 (34 mg, 0.034 mmol, 45.04%). m/z: 503.4 (M/2+H)⁺.

### Step 3

To a solution of 4-[2-(2,6-dioxomorpholin-4-yl) ethyl]morpholine-2,6-dione (0.037 mL, 0.209 mmol) in DMF (3 mL) was added compound 40-3 (21 mg, 0.021 mmol) and DIPEA (5.40 mg, 0.042 mmol). The mixture was stirred at room temperature for 2 h. The resulting solution was purified by reverse phase separation (C18 column, eluting with 0-45% acetonitrile in water with TFA) to afford the product PB040 (10.8 mg, 0.008 mmol, 40.40%). m/z: 640.4 (M/2+H)⁺.

Drug linker PB040 can be used to make a conjugate such as PA040.

### Example 11: Preparation of the Drug-Linker (PB041) Containing Two Sugar Units and a Cleavable Linker Attached to Exatecan

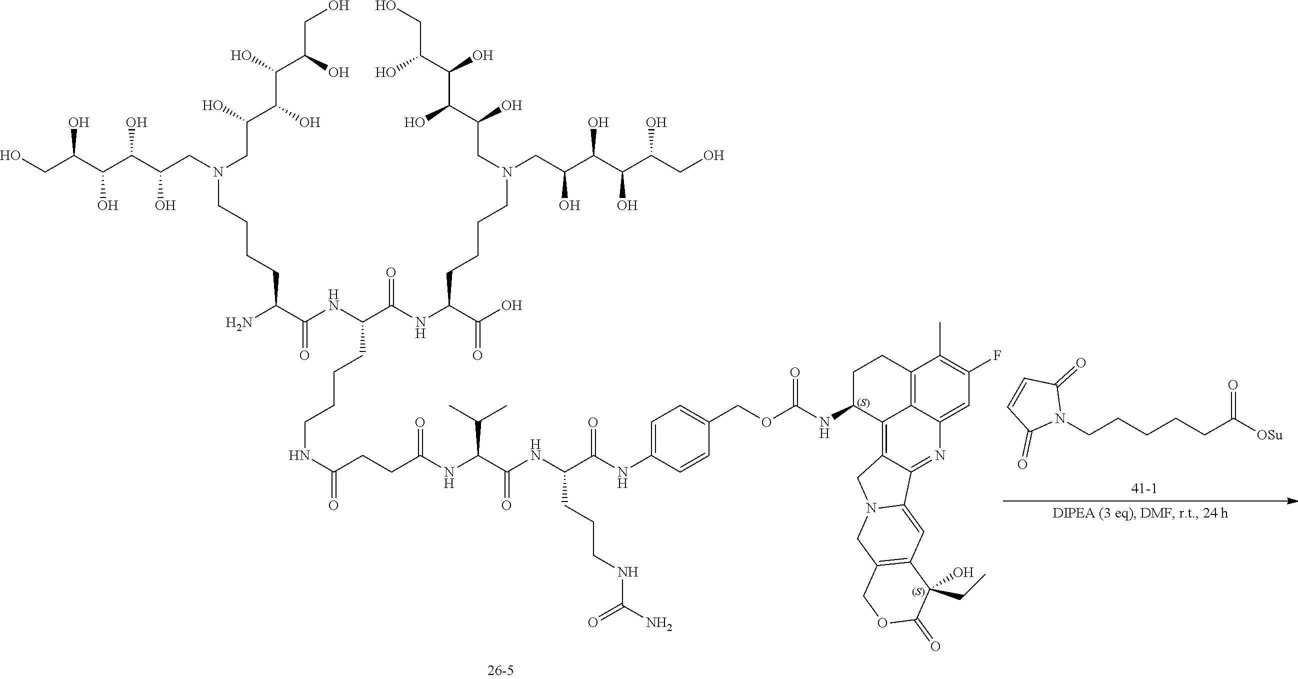

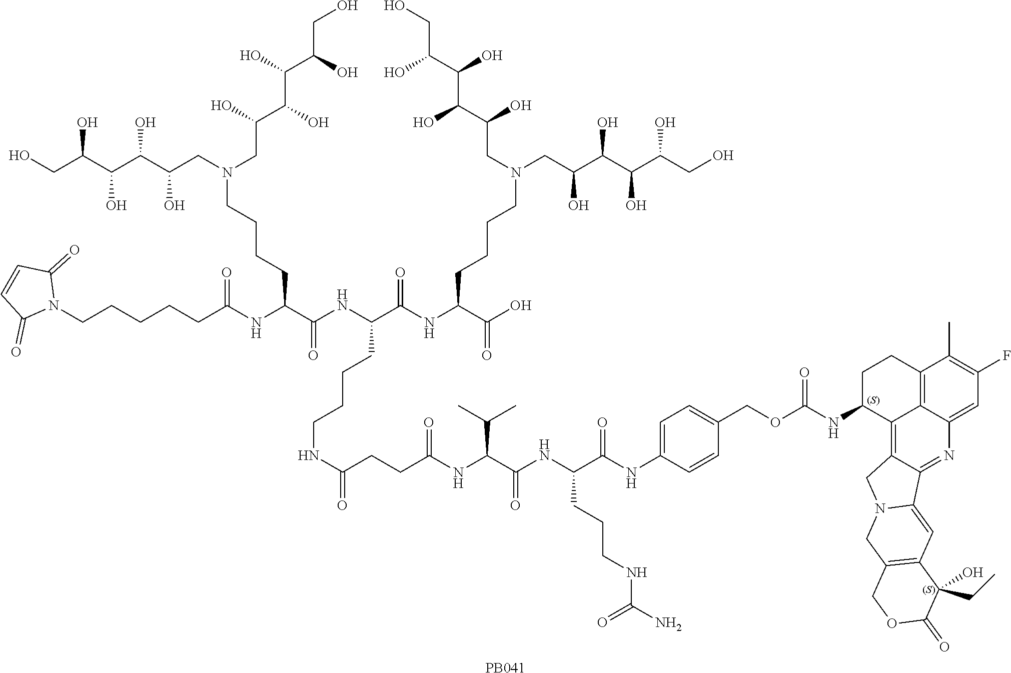

A maleimidylcaproyl Stretcher unit was attached to a Drug-Linker intermediate as follows:

A solution of compound 26-5 (25 mg, 0.013 mmol) and DIPEA (5.03 mg, 0.039 mmol) in anhydrous DMF (0.4 mL) was stirred at room temperature for 5 min, then a solution of Compound 41-1 (2,5-dioxopyrrolidin-1-yl 6-(2,5-dioxo-2,5-dihydro-1H-pyrrol-1-yl)hexanoate) (5.83 mg, 0.019 mmol) in anhydrous DMF (0.1 mL) was added dropwise by syringe over 2 min. The resulting solution was stirred for another 8 hr at r.t. until LCMS indicated that all starting amine was consumed and the mass of desired product was detected. The reaction solution was adjusted to pH 6-7 with diluted formic acid in acetonitrile, then purified directly by Prep-HPLC (eluting with gradient with 0.01% FA over 20 min) to yield PB041 (5.8 mg, 0.003 mmol, 21.14%) as a white solid.

LCMS, m/z=725.8 (M/3+H)⁺;

1H NMR (400 MHz, DMSO-d6): δ 9.72 (s, 1H), 8.31-8.15 (m, 3H), 8.09-8.04 (m, 2H), 7.98-7.96 (in, 1H), 7.96-7.86 (m, 1H), 7.78 (d, J=11.2 Hz, 1H), 7.65 (d, J=7.2 Hz, 2H), 7.36 (d, J=8.0 Hz, 2H), 7.32 (s, 1H), 7.00 (s, 2H), 6.54 (s, 1H), 6.09-6.02 (m, 1H), 5.47-5.45 (m, 4H), 5.30-5.29 (m, 3H), 5.08 (s, 2H), 4.36-4.23 (m, 3H), 4.18-4.13 (m, 1H), 4.13-4.08 (m, 0.5H), 3.92-3.80 (m, 1H), 3.74-3.69 (m, 1H), 3.67-3.56 (m, 10H), 3.05-2.88 (m, 15H), 2.64-2.59 (m, 1H), 2.36-2.33 (m, 12H), 2.24-1.97 (m, 14H), 1.91-1.82 (m, 5H), 1.78-1.61 (m, 14H), 1.53-1.34 (m, 15H), 1.34-1.11 (m, 17H), 0.89-0.86 (m, 11H) ppm. 19F NMR (400 MHz, DMSO-d6): δ −111 ppm.

Drug linker PB041 can be used to make a conjugate such as PA041.

### Example 12: Preparation of the Drug-Linker (PB050) Containing a PEG Unit and a Cleavable Linker Attached to Exatecan

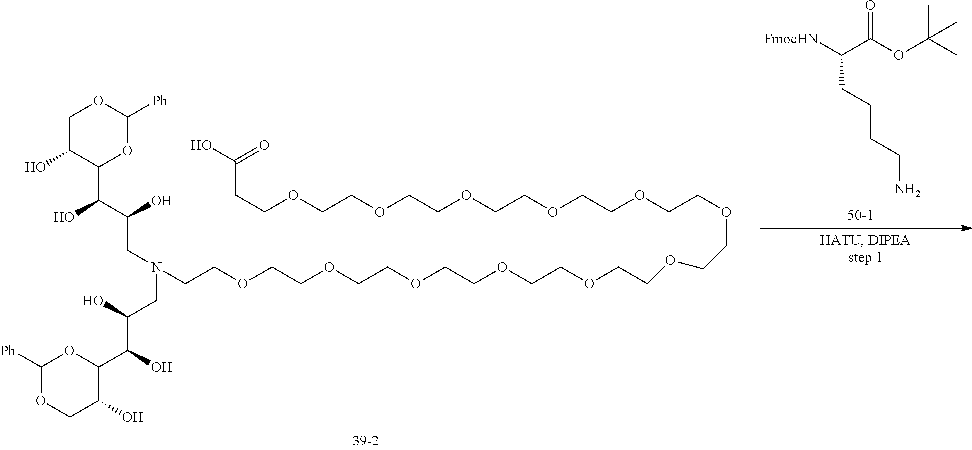

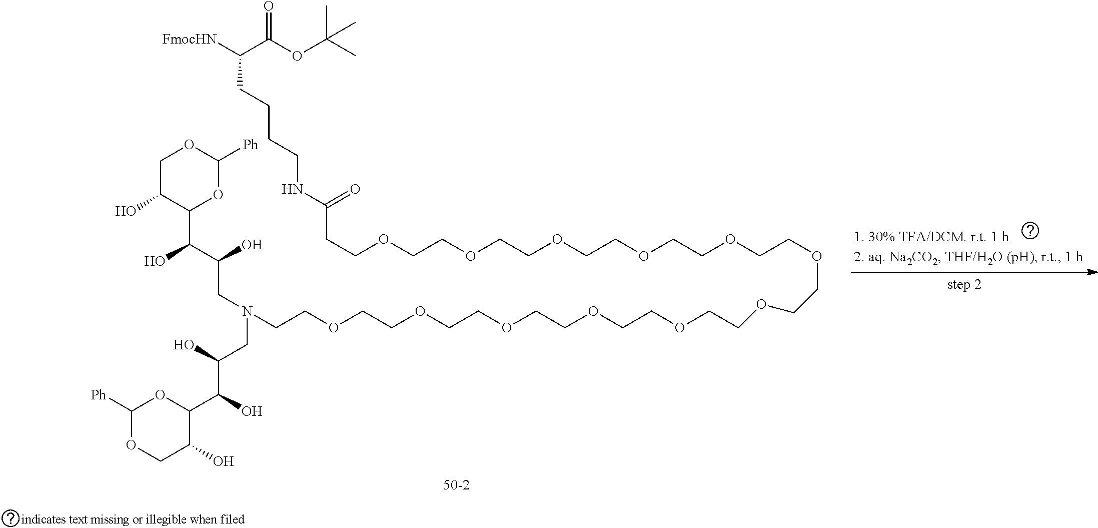

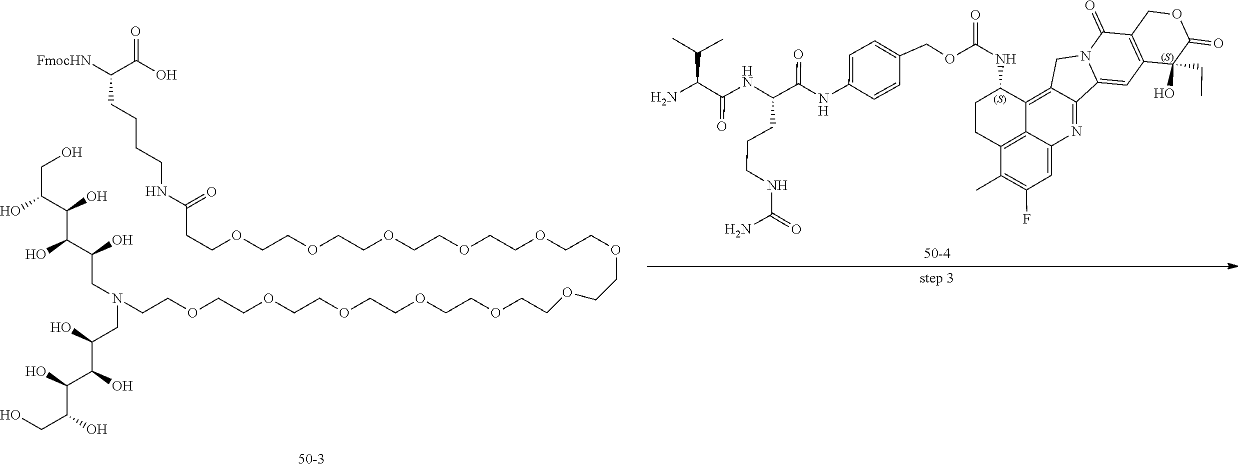

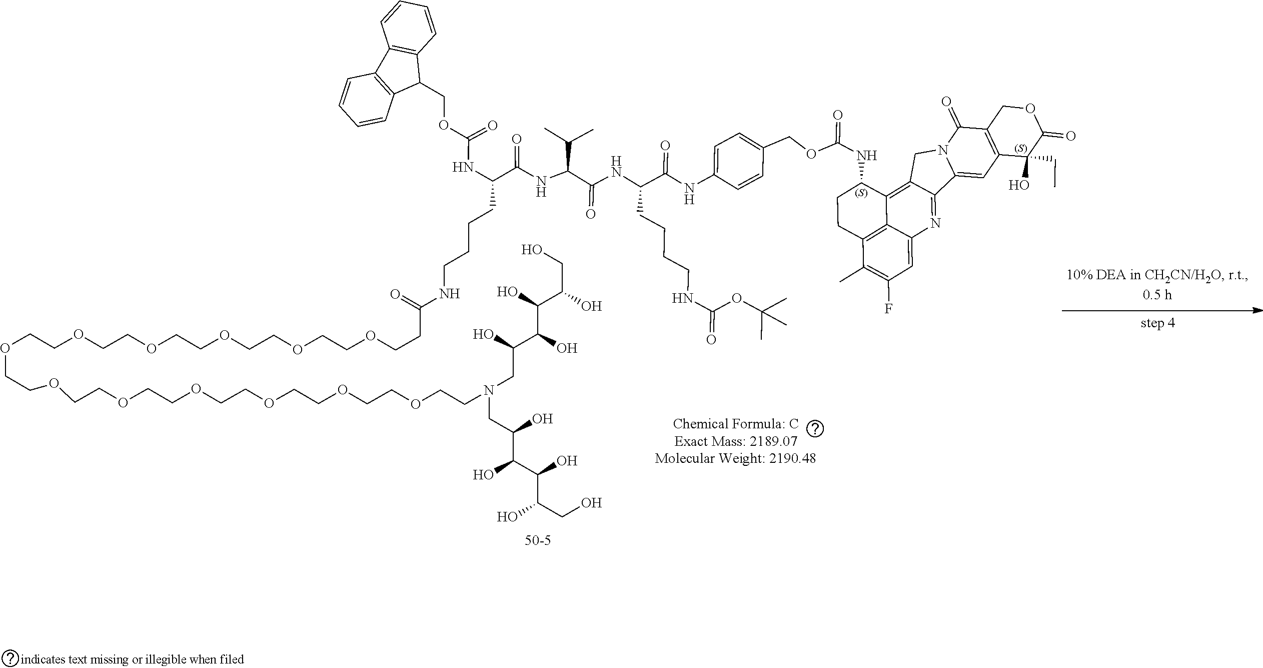

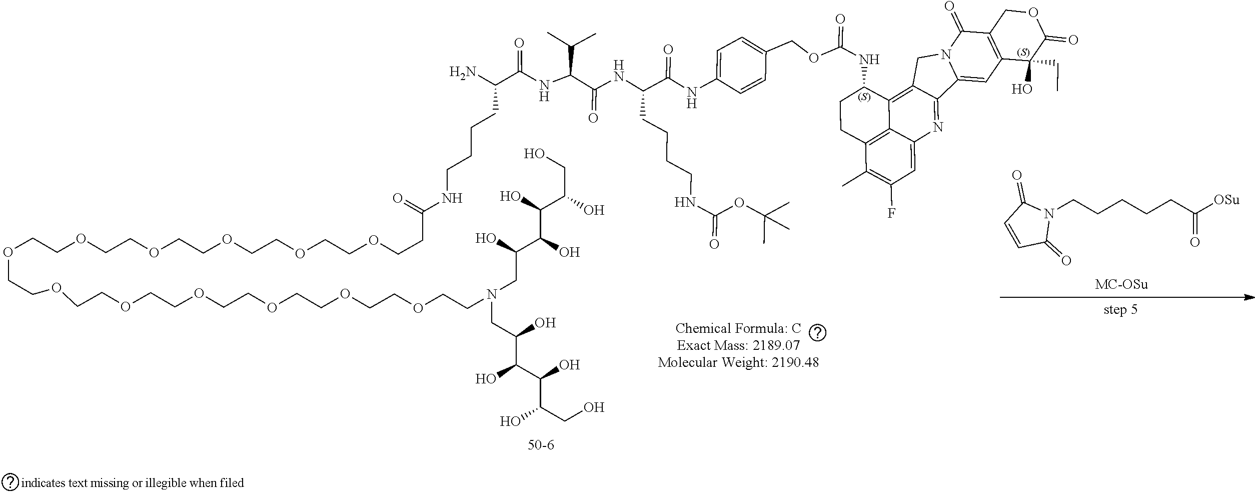

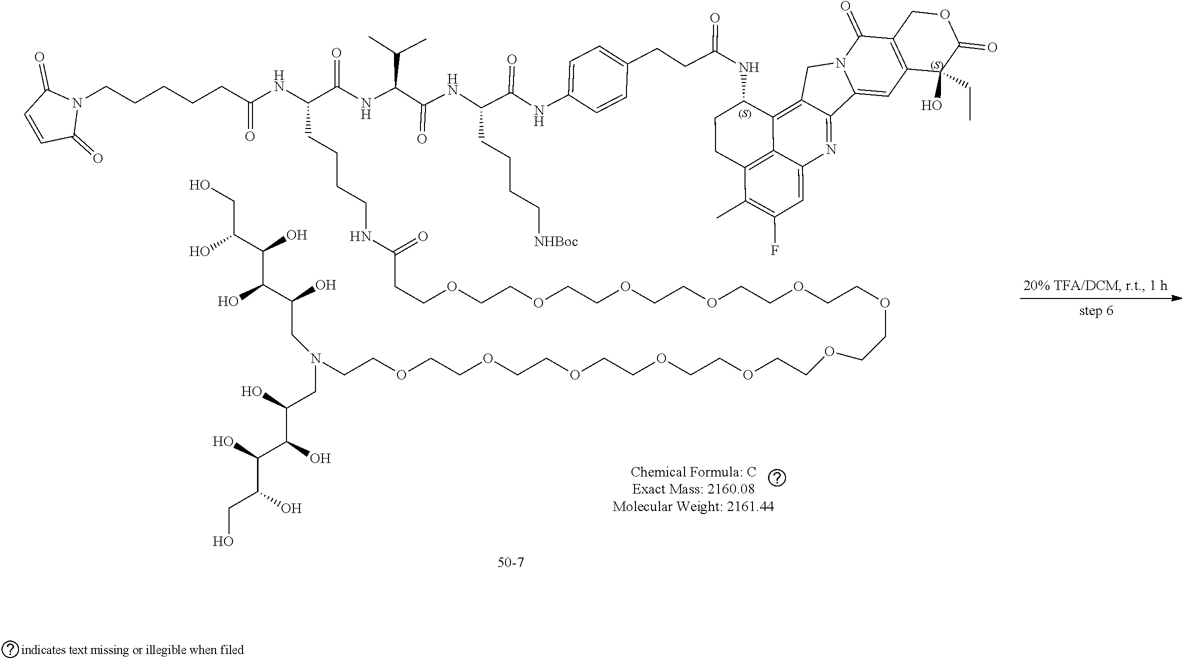

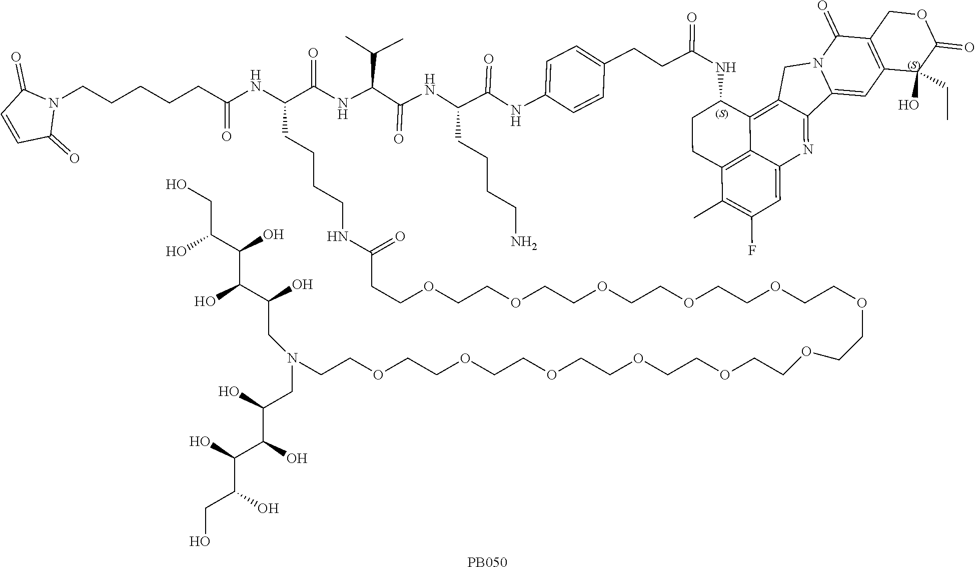

A Drug-Linker containing a PEG linker and a cleavable linker attached to exatecan (PB050) was prepared as follows:

### Step 1

A solution of compound 50-1 (56.74 mg, 0.134 mmol), HATU (60.99 mg, 0.160 mmol) and DIPEA (51.73 mg, 0.401 mmol) in anhydrous DMF (1.5 mL) was stirred at room temperature for 5 min, then compound 39-2 (150 mg, 0.134 mmol) was added. The resulting solution was stirred for another 1 hr at r.t. until LCMS indicated the reaction was complete. The reaction solution was purified directly by reverse phase liquid chromatography (40 g C18 column, eluting with 0-70% acetonitrile in water with 0.01% TFA over 15 min) to yield compound 50-2 (130 mg, 0.085 mmol, yield=63.62%) as a white solid. LCMS, ESI m/z=763.8 (M/2+H)⁺

### Step 2

A solution of compound 50-2 (130 mg, 0.085 mmol) in DCM (0.7 mL) was stirred at room temperature for 5 min, and then TFA (0.3 mL, 4.039 mmol) was added. The resulting solution was stirred for another 2 hr at r.t. until LCMS indicated the reaction was completed. The reaction solution was concentrated to dryness under vacuo, and the residue was then purified directly by reverse phase column chromatography (eluting with gradient with 0.01% FA over 20 min) to yield compound 50-3 (60 mg, 0.046 mmol, 54.43%) as a white solid. LCMS, m/z=725.8 (M/3+H)⁺;

1H NMR (400 MHz, DMSO-d6): δ 9.72 (s, 1H), 8.31-8.15 (m, 3H), 8.09-8.04 (m, 2H), 7.98-7.96 (m, 1H), 7.96-7.86 (m, 1H), 7.78 (d, J=11.2 Hz, 1H), 7.65 (d, J=7.2 Hz, 2H), 7.36 (d, J=8.0 Hz, 2H), 7.32 (s, 1H), 7.00 (s, 2H), 6.54 (s, 1H), 6.09-6.02 (m, 1H), 5.47-5.45 (m, 4H), 5.30-5.29 (m, 3H), 5.08 (s, 2H), 4.36-4.23 (m, 3H), 4.18-4.13 (m, 1H), 4.13-4.08 (m, 0.5H), 3.92-3.80 (m, 1H), 3.74-3.69 (m, 1H), 3.67-3.56 (m, 10H), 3.05-2.88 (m, 15H), 2.64-2.59 (m, 1H), 2.36-2.33 (m, 12H), 2.24-1.97 (m, 14H), 1.91-1.82 (m, 5H), 1.78-1.61 (m, 14H), 1.53-1.34 (m, 15H), 1.34-1.11 (m, 17H), 0.89-0.86 (m, 11H) ppm.

### Step 3

A solution of compound 50-3 (59 mg, 0.046 mmol), HATU (20.76 mg, 0.055 mmol) and DIPEA (17.61 mg, 0.137 mmol) in anhydrous DMF (1 mL) was stirred at room temperature for 5 min, and then compound 50-4 (41.51 mg, 0.046 mmol) was added. The resulting solution was stirred for another 1 hr at r.t. until LCMS indicated the reaction was complete. The reaction solution was purified directly by reverse phase liquid chromatography (40 g C18 column, eluting with 0-50% acetonitrile in water with 0.01% TFA over 15 min) to yield compound 50-5 (40 mg, 0.018 mmol, 40.12%) as a white solid. LCMS, m/z=697.6 ((M−100)/2+H)⁺; 1H NMR (400 MHz, DMSO-d6) δ 10.02 (s, 1H), 8.20-8.14 (m, 1H), 8.14-7.98 (m, 2H), 7.88 (d, J=7.6 Hz, 2H), 7.82-7.70 (m, 5H), 7.64-7.52 (m, 4H), 7.43-7.31 (m, 7H), 6.78-6.72 (m, 1H), 6.53 (s, 1H), 5.45-5.44 (m, 4H), 5.34-5.23 (m, 3H), 5.08 (s, 2H), 4.82-4.77 (m, 2H), 4.62-4.36 (m, 6H), 4.36-4.16 (m, 6H), 4.05-3.95 (m, 3H), 3.81-3.73 (m, 2H), 3.73-3.66 (m, 2H), 3.62-3.56 (m, 9H), 3.56-3.34 (m, 50H), 3.05-3.01 (m, 2H), 2.95-2.84 (m, 2H), 2.38 (s, 3H), 2.29 (t, J=6.0 Hz, 2H), 2.19-2.10 (m, 2H), 2.01-1.93 (m, 1H), 1.90-1.83 (m, 2H), 1.71-1.48 (m, 4H), 1.40-1.23 (m, 18H), 0.90-0.81 (m, 9H) ppm. One proton signal of TFA in included. 19F NMR (400 MHz, DMSO-d6), δ TFA at −73 ppm, Ar—F at −111 ppm

### Step 4

A solution of compound 50-5 (40 mg, 0.018 mmol) in CH₃CN (2 mL) and water (1 mL) was stirred at r.t. and diethyl amine anhydrous (0.002 mL, 0.018 mmol) was added. The resulting solution was stirred at r.t. for 2 h until LCMS showed a complete reaction. All the solvent was evaporated to yield a crude solid, which was suspended in more acetonitrile and evaporated again to remove diethyl amine completely. Then the residue was dissolved in acetonitrile and water, acidified with formic acid to pH 2-3 and let stand for 1 h; LCMS indicated all the lactone ring was closed back. Then the solution was lyophilized overnight to yield product compound 50-6 (36 mg, 0.018 mmol, 100.17%) as a pale yellow solid. This compound was used as such for the next step without any purification. LCMS: (crude, treated with formic acid, m/z=656.9 (M/3+H)⁺, 984.6 (M/2+H)⁺;

### Step 5

A solution of compound 50-6 (35.43 mg, 0.018 mmol) and DIPEA (6.97 mg, 0.054 mmol) in anhydrous DMF (0.8 mL) was stirred at room temperature for 5 min, then a solution of 2,5-dioxopyrrolidin-1-yl 6-(2,5-dioxo-2,5-dihydro-1H-pyrrol-1-yl) hexanoate (8.32 mg, 0.027 mmol) in anhydrous DMF (0.2 mL) was added dropwise by syringe. The resulting solution was stirred for another 1 hr at r.t. until LCMS indicated all starting amine was consumed and the mass of the desired product was detected. The resulting solution was acidified to pH 3-4 with formic acid and then purified directly by reverse phase flash chromatography (40 g C18 column, eluting with 0-70% acetonitrile in water with 0.01% T FA over 20 min) to give desired fractions, which was lyophilized to yield compound 50-7 (28 mg, 0.013 mmol, 71.96%) as a white solid. LCMS, m/z=687.9 ((M−100)/3+H)⁺;

### Step 6

To a solution of compound 50-7 (25 mg, 0.012 mmol) in DCM (4 mL) was added TFA (1 mL, 13.463 mmol) and then stirred at r.t. for 2 h until LCMS showed that the reaction was completed. TFA and DCM were evaporated under reduced pressure, then the residue was purified by Prep-HPLC (eluting with 0-100% acetonitrile in water with 0.01% TFA over 15 min) and the expected fractions were lyophilized to yield product PB050 as a white solid. LCMS, ESI m/z=516.3 (M/4+H)⁺, 687.9 (M/3+H)⁺; 1H NMR (400 MHz, DMSO-d6):

δ 10.06 (s, 1H), 8.16 (d, J=7.2 Hz, 1H), 8.07 (d, J=7.6 Hz, 1H), 7.99 (d, J=8.0 Hz, 1H), 7.84-7.80 (m, 2H), 7.77 (brs, 3H), 7.66-7.59 (m, 3H), 7.37 (d, J=8.8 Hz, 2H), 7.32 (s, 1H), 7.00 (s, 2H), 6.54 (s, 1H), 6.24 (s, 4H), 5.45 (s, 2H), 5.35-5.24 (m, 3H), 5.08 (s, 2H), 4.86-4.43 (m, 5H), 4.43-4.32 (m, 1H), 4.25-4.12 (m, 2H), 4.02-3.94 (m, 2H), 3.79-3.73 (m, 2H), 3.69-3.67 (m, 2H), 3.59-3.58 (m, 1H), 3.57-3.55 (m, 9H), 3.55-3.47 (m, 52H), 3.04-2.95 (m, 2H), 2.81-2.73 (m, 2H), 2.38 (s, 3H), 2.29 (t, J=6.4 Hz, 2H), 2.23-2.02 (m, 5H), 2.03-1.93 (m, 2H), 1.93-1.83 (m, 3H), 1.76-1.67 (m, 1H), 1.59-1.43 (m, 10H), 1.39-1.24 (m, 6H), 1.24-1.15 (m, 5H), 0.90-0.81 (m, 9H) ppm. 19F NMR (400 MHz, DMSO-d6): −111 ppm. Drug linker PB050 can be used to make a conjugate such as PA050.

### Example 13: Preparation of the Drug-Linker (PB082) Containing a PEG Unit and a Cleavable Linker Attached to Exatecan

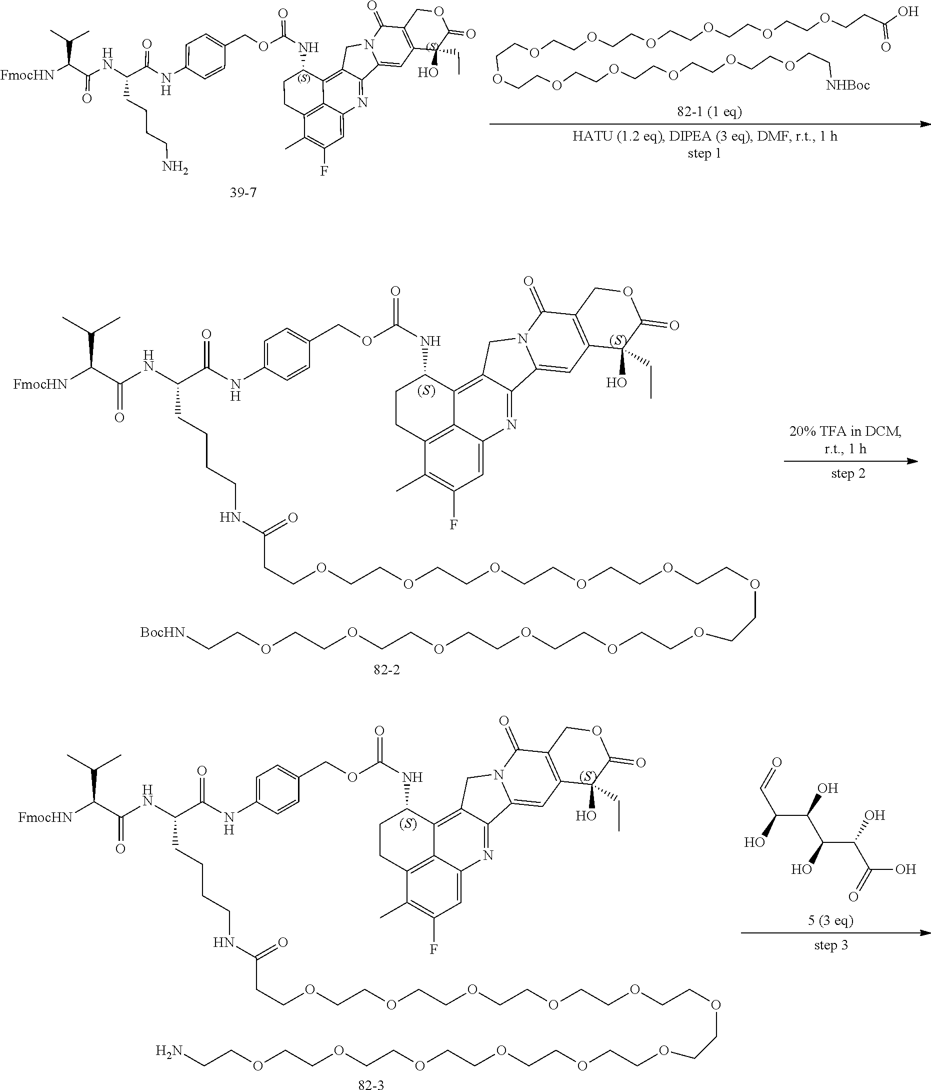

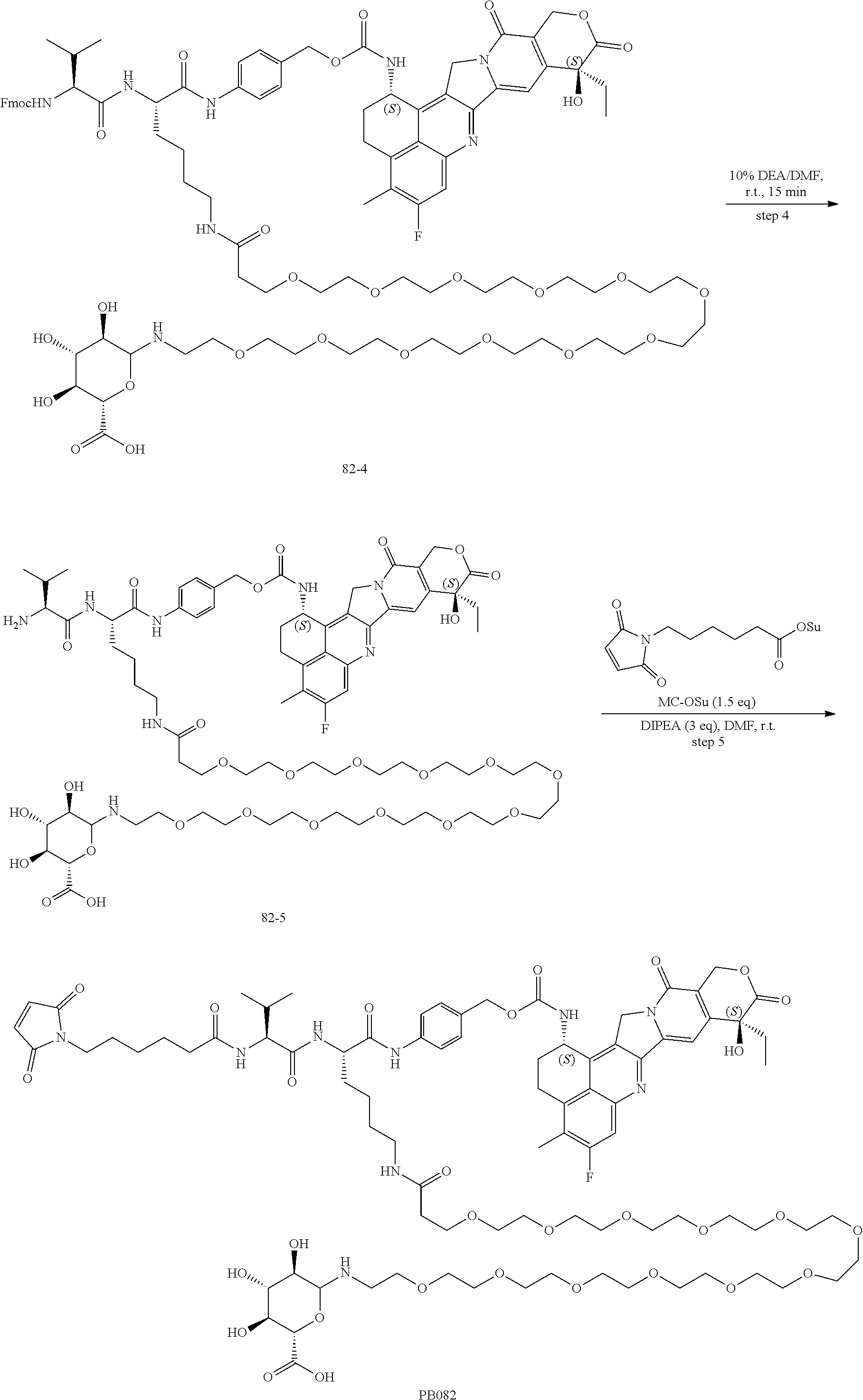

A Drug-Linker (PB082) containing a PEG unit and a cleavable linker attached to exatecan was prepared as follows:

### Step 1

A solution of compound 82-1 (173.53 mg, 0.242 mmol), HATU (110.30 mg, 0.290 mmol) and DIPEA (93.55 mg, 0.725 mmol) in anhydrous DMF (3 mL) was stirred at room temperature for 5 min, then compound 39-7 (250 mg, 0.242 mmol) was added. The resulting solution was stirred for another 2 hr at r.t. until LCMS indicated complete reaction. The reaction solution was purified directly by reverse phase liquid chromatography (40 g C18 column, eluting with 0-70% acetonitrile in water with 0.01% TFA over 15 m) to yield compound 82-2 (270 mg, 0.156 mmol, 64.41%) as a pale yellow solid.

### Step 2

A solution of compound 82-2 (870 mg, 0.502 mmol) in DCM (4 mL) was stirred at room temperature, then TFA (1 m, 13.463 mmol) was added, and the light yellow solution was stirred for 1 h. until LCMS of the solution showed that the deprotection was completed. The solvent and TFA were evaporated, then the residue was purified directly by reverse phase liquid chromatography (40 g C18 column, eluting with 0-20% acetonitrile in water with 0.01% TFA for 10 mi) to yield the expected fractions, which were freeze-dried to yield compound 82-3 (650 mg, 0.398 mmol, 79.29%) as a white solid. LCMS, m/z=545.5 (M/3+H)⁺, 817.6 (M/2+H)⁺;

### Step 3

A mixture of compound 82-3 (220 mg, 0.213 mmol) and (2S,3S,4S,5R)-2,3,4,5-tetrahydroxy-6-oxohexanoic acid (123.90 mg, 0.638 mmol) in methanol (6 ml) was heated at 50° C. for 8 hrs to achieve complete conversion. Then the suspension was concentrated to remove methanol, the residue was dissolved in DMF and purified by reverse phase column chromatography (40 g C18 column, eluting with 0-50% acetonitrile in water with 10 mM ammonium bicarbonate over 15 min) to collect the desired fractions, which were freeze-dried to yield product 82-4 as a white solid. LCMS, m/z=604.2 (M/3+H)⁺, 905.6 (M/2+H)⁺,

### Step 4

A solution of compound 82-4 (100 mg, 0.055 mmol) in DMF (0.9 mL) was stirred at r.t. and diethyl amine anhydrous (0.1 mL, 0.971 mmol) was added. The resulting solution was stirred at r.t. for 15 min. The LCMS of the reaction mixture showed that the reaction was completed. Most of the diethyl amine and other volatiles were evaporated, then the residue was acidified to pH 3-4 with formic acid, and then purified by reverse phase flash chromatography (0-40% acetonitrile in water with 0.01% TFA) to yield expected compound 82-5 (35 mg, 0.022 mmol, 39.90%) as a white solid. LCMS, m/z=794.5 (M/2+H)⁺, 530.2 (M/2+H)⁺;

### Step 5

A solution of compound 82-5 (150 mg, 0.083 mmol) and DIPEA (8.53 mg, 0.066 mmol) in anhydrous DMF (1 mL) was stirred at room temperature for 5 min, then a solution MC-OSu (10.20 mg, 0.033 mmol) in anhydrous DMF (1 mL) was added dropwise by syringe. The resulting solution was stirred for another 6 hr at r.t. until the desired product was detected as major new peak, along with little byproduct The resulting solution was acidified by formic acid to pH3-4, then purified directly by Prep-HPLC (eluting with gradient with 0.01% TFA over 20 min) to yield product PB082 (10 mg, 0.006 mmol, 25.52%) as a white solid.

LCMS, m/z=594.5 (M/3+H)⁺, 891.3 (M/2+H)⁺ 891.3 (M/2+H)⁺

1H NMR: (500 MHz, DMSO-d6) δ 9.96 (s, 1H), 8.07-8.02 (m, 2H), 7.83-7.81 (m, 2H), 7.77 (d, J=10.5 Hz, 1H), 7.59 (d, J=8.0 Hz, 2H), 7.36 (d, J=8.5 Hz, 2H), 7.31 (s, 1H), 6.99 (s, 2H), 6.67 (s, 1H), 6.53 (s, 1H), 5.84-5.79 (m, 1H), 5.45 (s, 2H), 5.33-5.23 (m, 3H), 5.08 (s, 2H), 4.35-4.24 (m, 2H), 4.18-4.13 (m, 1H), 4.10-4.04 (m, 1H), 3.92-3.91 (m, 1H), 3.73-3.67 (m, 2H), 3.58-3.54 (m, 6H), 3.52-3.45 (m, 44H), 3.21-3.09 (m, 6H), 3.03-2.99 (m, 2H), 2.38 (s, 3H), 2.28 (t, J=6.5 Hz, 2H), 2.21-2.08 (m, 4H), 2.00-1.90 (m, 1H), 1.90-1.82 (m, 2H), 1.74-1.65 (m, 1H), 1.65-1.57 (m, 1H), 1.52-1.45 (m, 4H), 1.45-1.27 (m, 5H), 1.24-1.15 (m, 2H), 0.90-0.81 (m, 9H) ppm.

Drug linker PB082 can be used to make a conjugate such as PA082.

### Example 14: Preparation of the Drug-Linker (PB083) Containing a PEG Unit and a Cleavable Linker Attached to Exatecan

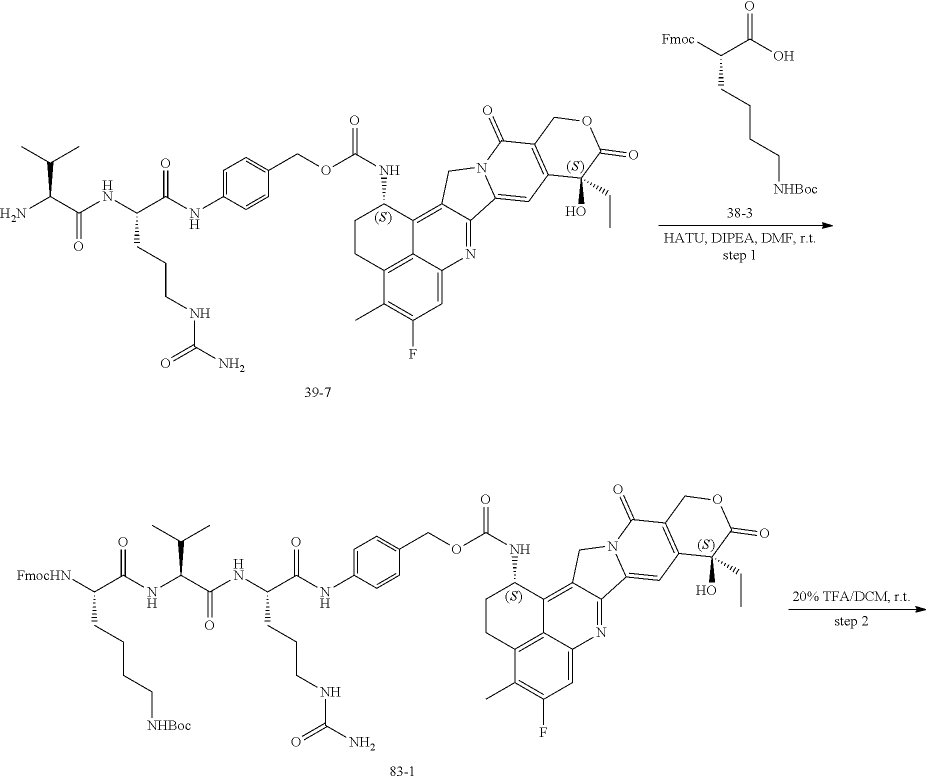

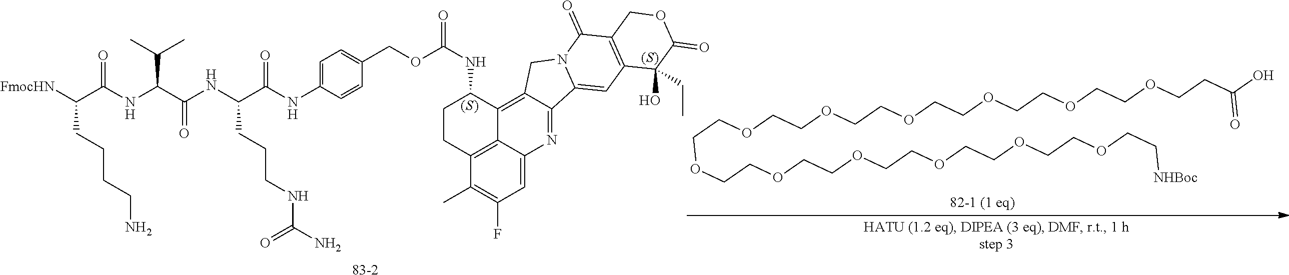

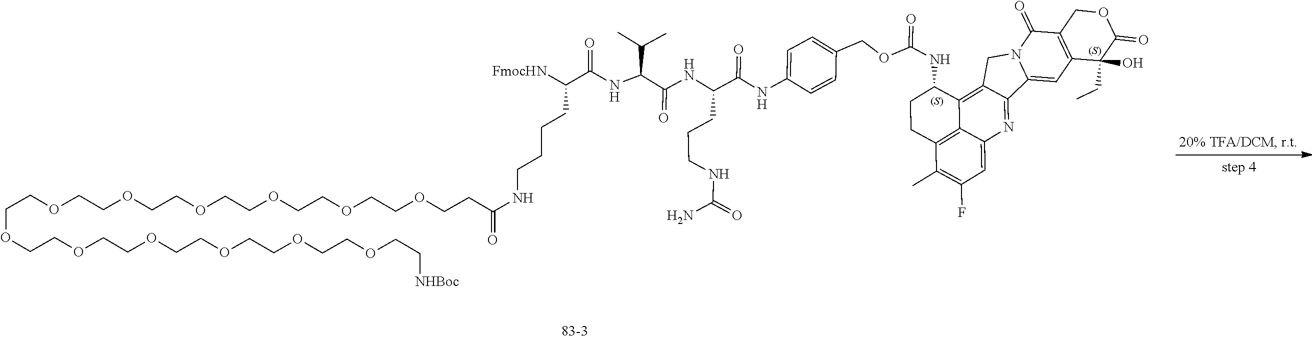

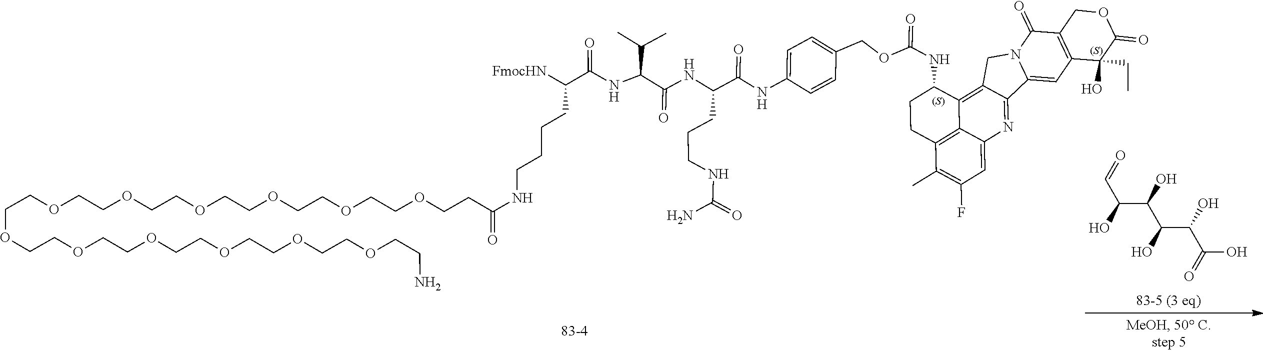

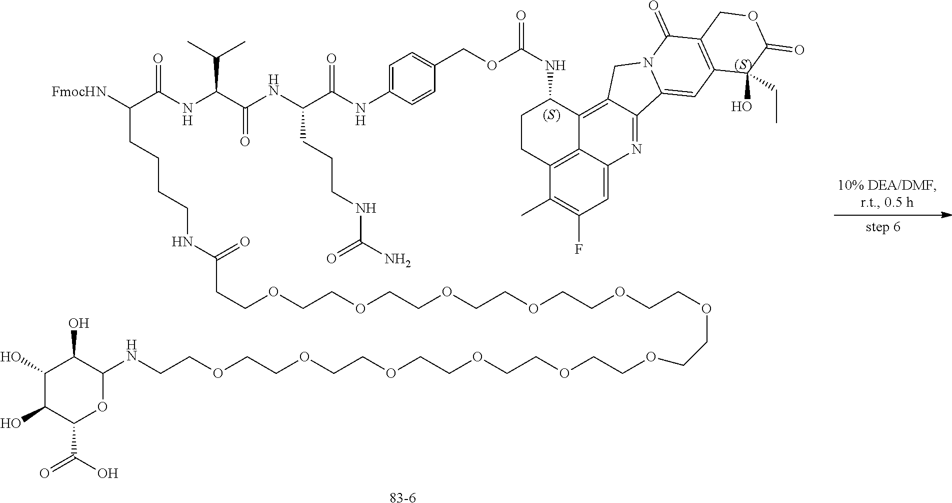

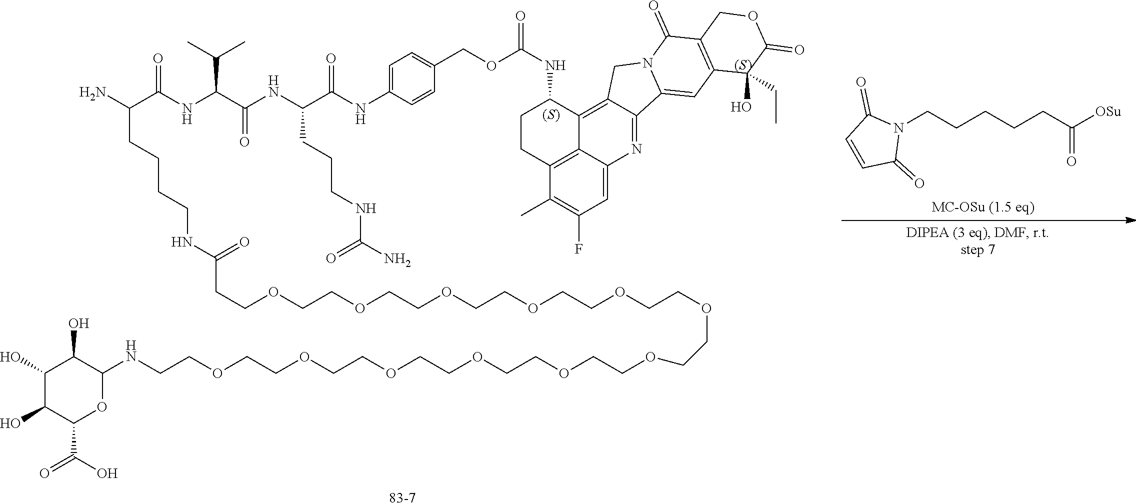

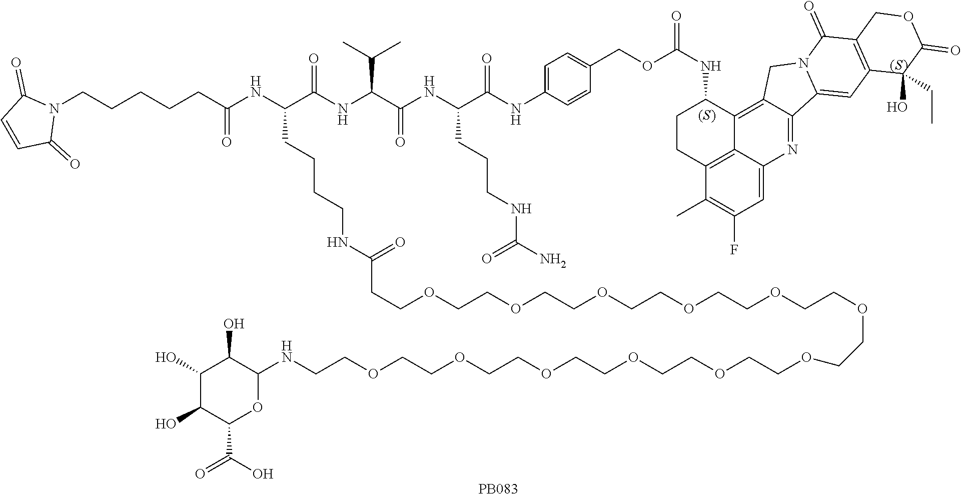

A Drug-Linker containing a PEG unit and a cleavable linker attached to exatecan (PB083) was prepared as follows:

### Step 1

A solution of compound 38-3 (0.190 mL, 0.499 mmol), HATU (284.86 mg, 0.749 mmol) and DIPEA (193.29 mg, 1.498 mmol) in anhydrous DMF (3 mL) was stirred at room temperature for 5 min, and then a solution of compound 38-7 (420 mg, 0.499 mmol) in anhydrous DMF (2 mL) was added. The resulting solution was stirred for another 1.5 hr at r.t. until LCMS indicated the reaction was complete. The reaction solution was purified directly by reverse phase liquid chromatography (40 g C18 column, eluting with 0-100% acetonitrile in water with 0.01% TFA over 15 min then 100% methanol for 5 min) to yield compound 83-1 (330 mg, 0.256 mmol, 51.16%) as a pale yellow solid. LCMS, m/z=596.4 ((M−100)/2+H.

1H NMR (400 MHz, DMSO-d6) δ 10.05 (s, 1H), 8.14 (d, J=7.6 Hz, 1H), 8.06 (d, J=9.2 Hz, 1H), 7.89 (d, J=7.6 Hz, 2H), 7.78 (d, J=11.2 Hz, 1H), 7.73-7.71 (m, 3H), 7.60 (d, J=8.4 Hz, 2H), 7.53 (d, J=8.4 Hz, 1H), 7.43-7.31 (m, 8H), 6.81-6.73 (m, 1H), 6.03-5.93 (m, 1H), 5.54-5.37 (m, 3H), 5.37-5.24 (m, 3H), 5.08 (s, 2H), 4.44-4.34 (m, 1H), 4.34-4.23 (m, 4H), 4.06-3.97 (m, 1H), 3.29-3.13 (m, 1H), 3.13-2.98 (m, 2H), 2.98-2.81 (m, 3H), 2.38 (s, 3H), 2.26-2.17 (m, 2H), 2.09-1.96 (m, 1H), 1.96-1.73 (m, 2H), 1.67-1.54 (m, 3H), 1.45-1.23 (m, 17H), 0.89-0.81 (m, 9H) ppm.

### Step 2

To a solution of compound 83-1 (330 mg, 0.256 mmol) in DCM (4 mL) was added TFA (1 mL, 6.228 mmol), and the solution was stirred at r.t. for 1 h until LCMS of the solution showed that the reaction was completed. Solvents were evaporated under reduced pressure, then the residue was purified by reverse phase liquid chromatography (40 g C18 column, eluting with 0-40% acetonitrile in water with 0.01% TFA for 10 min) to yield product compound 83-2 (300 mg, 0.252 mmol, 98.55%) as a pale yellow solid. LCMS, m/z=596.3 (M/2+H)⁺;

### Step 3

A solution of compound 82-1 (180.77 mg, 0.252 mmol), HATU (114.90 mg, 0.302 mmol) and DIPEA (97.45 mg, 0.755 mmol) in anhydrous DMF (1.5 mL) was stirred at room temperature for 5 min, then a solution compound 83-2 (300 mg, 0.252 mmol) in anhydrous DMF (0.5 mL) was added. The resulting solution was stirred for another 2 hrs at r.t. until LCMS indicated a complete reaction. The reaction solution was purified directly by reverse phase liquid chromatography (40 g C18 column, eluting with 0-80% acetonitrile in water with 0.01% TFA over 15 min) to yield compound 83-3 (270 mg, 0.143 mmol, 56.70%) as a white solid. LCMS: m/z=946.1 (M/2+H)⁺;

### Step 4

A solution compound 83-3 (450 mg, 0.238 mmol) in DCM (8 mL) was stirred at r.t., then TFA (2 mL, 26.925 mmol) was added to the solution. The resulting yellow solution was stirred for 1 h to achieve complete deprotection. The completed solution was concentrated to remove DCM and TFA, then the brown residue was purified by reverse phase liquid chromatography (40 g C18 column, eluting with 0-50% acetonitrile in water with 0.01% TFA for 15 min) to yield expected product compound 83-4 as a white solid. LCMS, m/z=597.8 (M/3+H)⁺, 896.6 (M/2+H)⁺;

### Step 5

A reaction mixture compound 83-4 (250 mg, 0.140 mmol), compound 83-5 (81.30 mg, 0.419 mmol), HOAc (0.025 mL, 0.140 mmol) in methanol (5 mL) was stirred at 50° C. under N2 for 18 hrs. Then LCMS indicated most of compound 83-5 was consumed and the desired product was detected. The solvents were evaporated, and the residue was purified by C18 reversed-phase chromatography to yield the desired compound 83-6 (100 mg, 0.051 mmol, 36.42%). LCMS, m/z=656.3 (M/3+H)⁺, 983.8 (M/2+H)⁺;

### Step 6

The solution of compound 83-6 (100 mg, 0.051 mmol) in DMF (0.9 mL) was stirred at r.t. and diethyl amine anhydrous (0.1 mL, 0.971 mmol) was added. The resulting solution was stirred at r.t. for 15 min until LCMS showed the reaction was complete and the mass of the desired product was detected. Then the crude product was purified by reverse phase liquid chromatography (40 g C18 column, eluting with 0-50% acetonitrile in water with 0.01% TFA for 15 min) to yield expected product compound 83-7 as a white solid LCMS, m/z=582.5 (M/3+H)⁺;

### Step 7

A solution of compound 83-7 (50 mg, 0.029 mmol) and DIPEA (11.09 mg, 0.086 mmol) in anhydrous DMF (2 mL) was stirred at room temperature for 5 min, then a solution of MC-OSu (13.25 mg, 0.043 mmol) in anhydrous DMF (2 mL) was added dropwise by syringe. The resulting solution was stirred for another 6 hr at r.t. until LCMS indicated all starting amine was consumed and the desired product was detected. The resulting solution was acidified by adding formic acid to pH3-4, then purified directly by Prep-HPLC (eluting with gradient with 0.01% TFA over 20 min) to yield product PB083 (21 mg, 0.011 mmol, 37.81%) as a white solid. LCMS, m/z=485.7 (M/4+H)⁺, 646.9 (M/3+H)⁺

1H NMR: δ 10.04 (s, 1H), 8.14-8.05 (m, 2H), 7.96 (d, J=7.6 Hz, 1H), 7.83-7.76 (m, 2H), 7.67 (d, J=7.6 Hz, 1H), 7.60 (d, J=8.4 Hz, 2H), 7.36 (d, J=8.4 Hz, 2H), 7.31 (s, 1H), 6.99 (s, 2H), 6.53 (brs, 1H), 6.05-5.97 (m, 1H), 5.45-5.43 (m, 4H), 5.34-5.24 (m, 3H), 5.08 (s, 2H), 4.43-4.32 (m, 1H), 4.27-4.17 (m, 3H), 4.05-4.02 (m, 1H), 3.87-3.80 (m, 1H), 3.71-3.64 (m, 2H), 3.57-3.47 (m, 53H), 3.16-3.06 (m, 2H), 3.06-2.92 (m, 6H), 2.38 (s, 3H), 2.29 (t, J=6.4 Hz, 2H), 2.24-2.11 (m, 4H), 2.03-1.83 (m, 4H), 1.74-1.56 (m, 3H), 1.50-1.35 (m, 9H), 1.35-1.14 (m, 5H), 0.89-0.81 (m, 9H) ppm.

Drug linker PB083 can be used to make a conjugate such as PA083.

### Example 15: Preparation of the Drug-Linker (PB084) Containing a PEG Unit and a Cleavable Linker Attached to MMAE

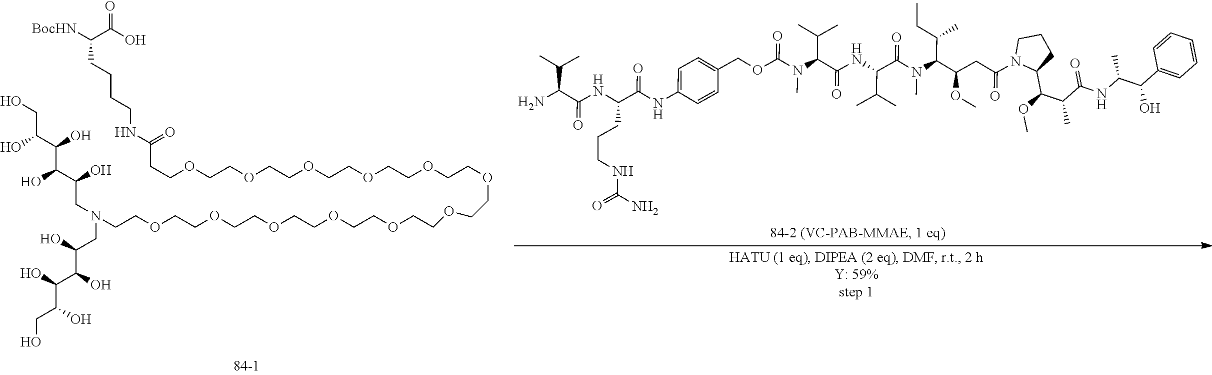 [SMILES: CC(C)(C)OC(=O)N[C@@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

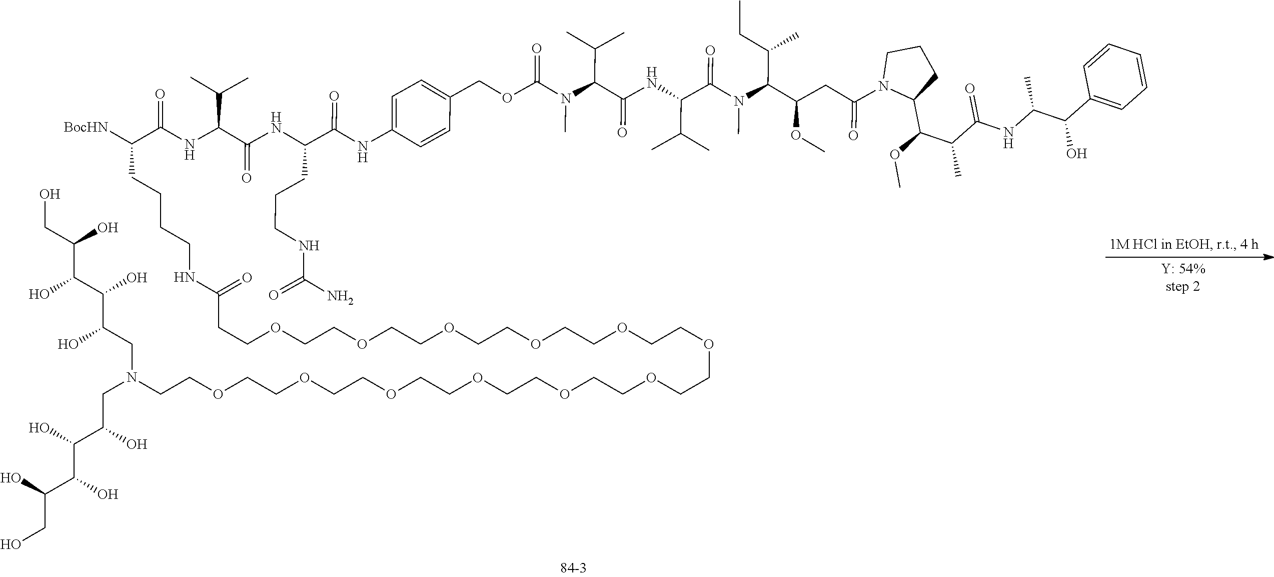 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc1)C(C)C]

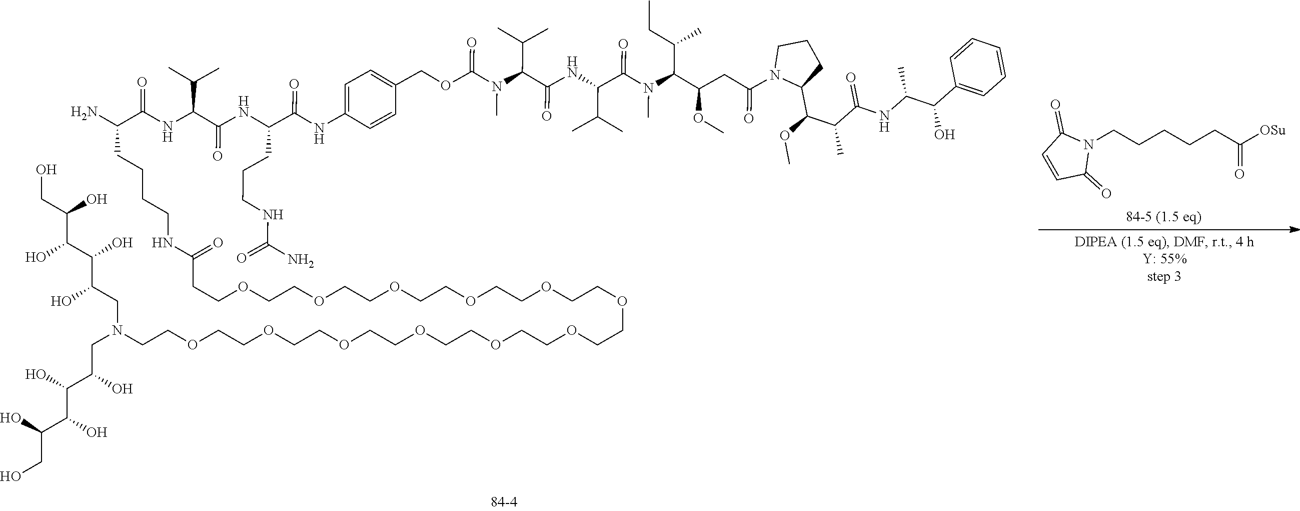

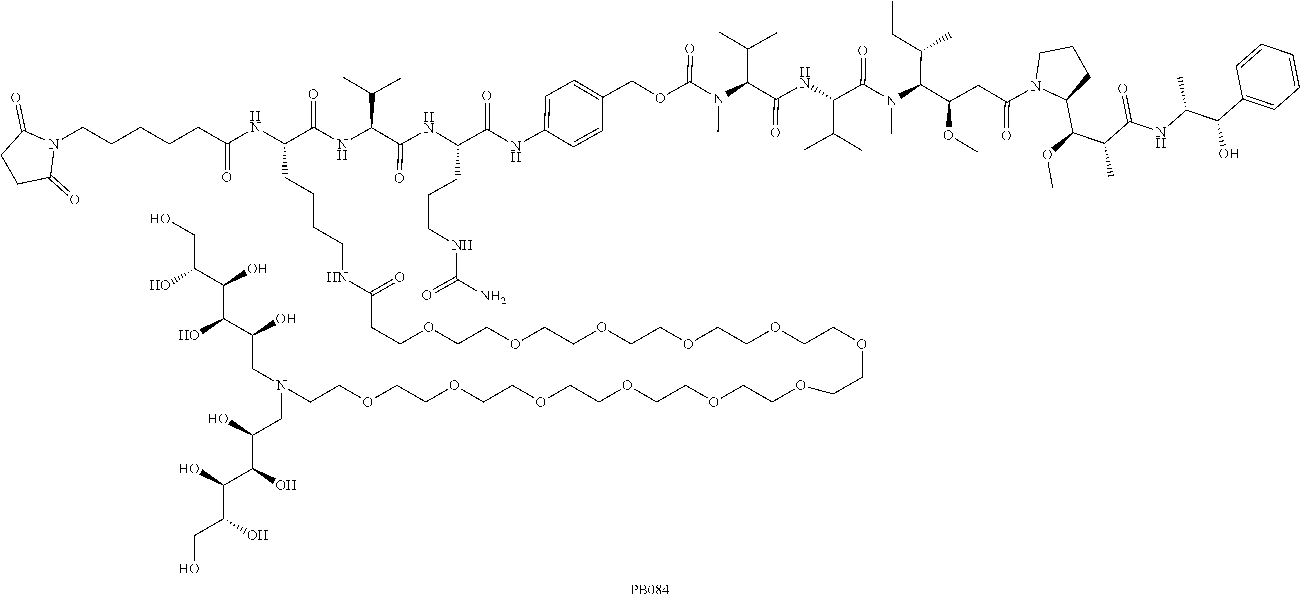

A Drug-Linker containing a PEG unit and a cleavable linker attached to MMAE (PB084) was prepared as follows:

### Step 1

A solution of compound 84-1 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]—N-methylcarbamate (84-1, 500 mg, 0.445 mmol)), compound 84-2 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (84-2, 522.71 mg, 0.445 mmol)) and DIPEA (172.31 mg, 1.336 mmol) in anhydrous DMF (1 mL) was stirred at room temperature, then a solution of HATU (169.29 mg, 0.445 mmol) in anhydrous DMF (1 mL) was added dropwise by syringe over 5 min. After addition, the resulting solution was stirred for another 2 h until LCMS indicated complete reaction. Then the completed reaction solution was purified directly by reverse phase flash chromatography (0.01% TFA) to yield compound 84-3 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-3, 600 mg, 0.263 mmol, 59.11%)) as a white solid. ESI m/z: 760.8 (M/3+H)⁺.

### Step 2

To the solution of compound 84-3 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-3, 580 mg, 0.254 mmol)) in ethanol (3 mL) was added 2M HCl in ethanol (3 mL, 0.254 mmol). Then the resulting pale yellow solution was stirred at room temperature for 4 h until LCMS showed that starting material was substantially consumed. The completed solution was cooled in ice water and neutralized with aqueous sodium bicarbonate solution. Solvent was removed under reduced pressure and the residue in the water layer was purified by reverse phase flash chromatography (0.01% TFA) to yield product 84-4 {4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-4, 300 mg, 0.138 mmol, 54.20%) as a white solid. ESI m/z: 727.3 (M/3+H)⁺, 718.3 (fragment piece, MMAE), 473.5 (linker fragment, ((2178-717-28-18)/3+H)⁺). ¹H NMR (400 MHz, DMSO-d6) δ 10.11 (s, 1H), 8.97-8.80 (m, 1H), 8.43 (d, J=8.8 Hz, 1H), 8.32 (d, J=7.2 Hz, 1H), 8.24-8.06 (m, 4H), 7.96-7.86 (m, 1.5H), 7.66 (d, J=8.8 Hz, 0.5H), 7.58 (d, J=7.6 Hz, 2H), 7.35-7.24 (m, 6H), 7.18-7.16 (m, 1H), 6.07 (s, 1H), 5.55-5.32 (m, 4H), 5.12-4.95 (m, 2H), 4.87-4.37 (m, 9H), 4.29-4.23 (m, 2H), 4.04-3.93 (m, 4H), 3.88-3.84 (m, 1H), 3.82-3.77 (m, 2H), 3.69-3.67 (m, 2H), 3.62-3.56 (m, 8H), 3.53-3.46 (m, 44H), 3.30-3.12 (m, 14H), 3.03-2.83 (m, 10H), 2.46-2.39 (m, 1H), 2.32-2.23 (m, 3H), 2.15-1.85 (m, 5H), 1.85-1.55 (m, 6H), 1.55-1.24 (m, 11H), 1.06-0.97 (m, 8H), 0.92-0.75 (m, 29H) ppm. Two protons of carboxyl group on TFA were revealed.

### Step 3

A solution of compound 84-4 {4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-4, 200 mg, 0.092 mmol) and compound 84-5 (2,5-dioxopyrrolidin-1-yl 6-(2,5-dioxo-2,5-dihydro-1H-pyrrol-1-yl)hexanoate (84-5, 42.39 mg, 0.138 mmol)) in anhydrous DMF (4 mL) was stirred at room temperature for 5 min, then a solution of DIPEA (23.67 mg, 0.184 mmol) in DMF (1 mL) was added dropwise by syringe over 5 min. After addition, the resulting solution was stirred for another 4 hr until LCMS indicated the starting amine was substantially consumed. Then the reaction solution was purified by Prep-HPLC (0.01% FA) to yield the desired fractions, which were freeze-dried to yield PB084 ({4-[(2S)-5-(carbamoylamino)-2-[(2S)-2-[(2S)-2-[6-(2,5-dioxo-2,5-dihydro-1H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB084, 120 mg, 0.051 mmol, 55.11%)) as a white solid. ESI m/z: 1187.2 (M/3+H)⁺, 791.7 (M/3+H)⁺, 718.5 (fragment piece, MMAE), 538.2 (linker fragment, (M−717−28−18)/3+H)⁺). Retention time 6.558 min (HPLC). ¹H NMR (400 MHz, DMSO-d6) δ 10.04 (s, 1H), 8.36-8.28 (m, 0.5H), 8.13 (d, J=6.8 Hz, 1H), 8.13-8.06 (m, 0.5H), 7.96 (d, J=8.0 Hz, 1H), 7.91 (d, J=8.4 Hz, 0.5H), 7.84-7.80 (m, 1H), 7.68-7.65 (m, 1.5H), 7.59-7.56 (m, 2H), 7.34-7.24 (m, 6H), 7.20-7.16 (m, 1H), 6.99 (s, 2H), 5.99 (d, J=5.6 Hz, 1H), 5.44-5.43 (m, 3H), 5.37 (d, J=4.8 Hz, 0.5H), 5.12-4.95 (m, 2H), 4.78-4.40 (m, 12H), 4.35-4.17 (m, 3.5H), 4.04-3.91 (m, 3H), 3.79-3.55 (m, 12H), 3.54-3.46 (m, 48H), 3.35-3.11 (m, 14H), 3.06-2.83 (m, 10H), 2.43-2.39 (m, 1H), 2.31-2.22 (m, 3H), 2.14-1.16 (m, 30H), 1.05-0.97 (m, 6H), 0.89-0.75 (m, 27H) ppm.

Drug linker PB084 can be used to make a conjugate such as PA084.

### Example 16: Preparation of the Drug-Linker (PB085) Containing a PEG Unit and a Cleavable Linker Attached to SN-38

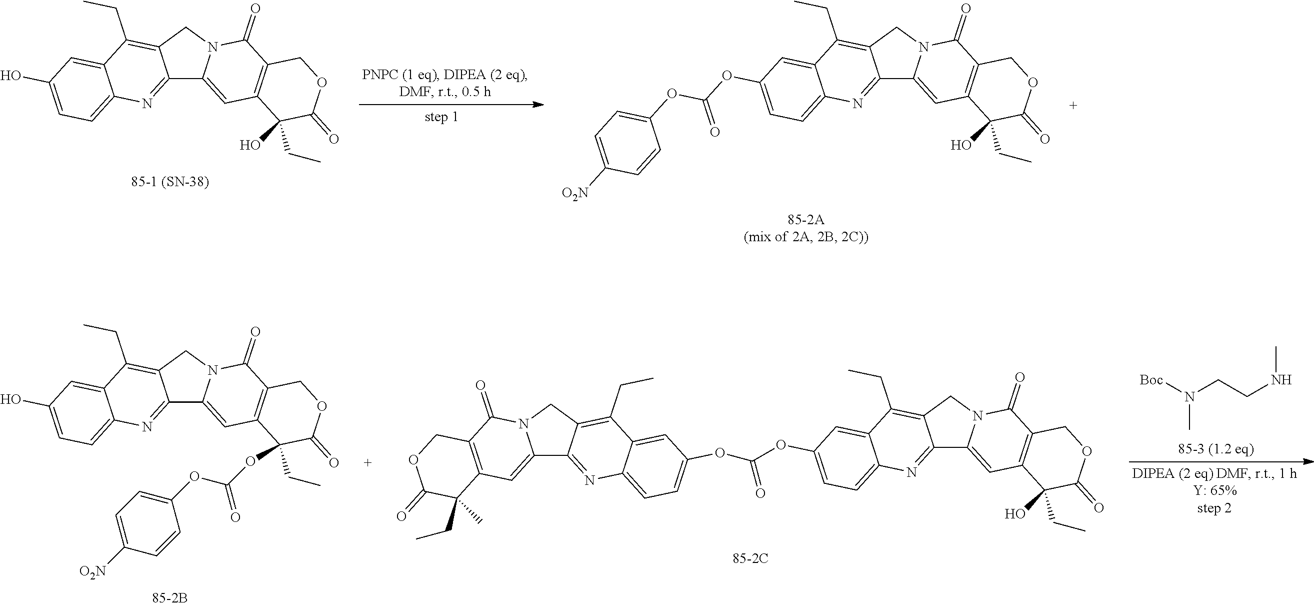 [SMILES: CCc1c2c(nc3ccc(O)cc13)-c1cc3c(c(=O)n1C2)COC(=O)[C@]3(O)CC ; CCc1c2c(nc3ccc(OC(=O)Oc4ccc([N+](=O)[O-])cc4)cc13)-c1cc3c(c(=O)n1C2)COC(=O)[C@]3(O)CC ; CCc1c2c(nc3ccc(O)cc13)-c1cc3c(c(=O)n1C2)COC(=O)[C@@]3(CC)OC(=O)Oc1ccc([N+](=O)[O-])cc1 ; CCc1c2c(nc3ccc(OC(=O)Oc4ccc5nc6c(c(CC)c5c4)Cn4c-6cc5c(c4=O)COC(=O)[C@]5(O)CC)cc13)-c1cc3c(c(=O)n1C2)COC(=O)[C@]3(C)CC ; CNCCN(C)C(=O)OC(C)(C)C]

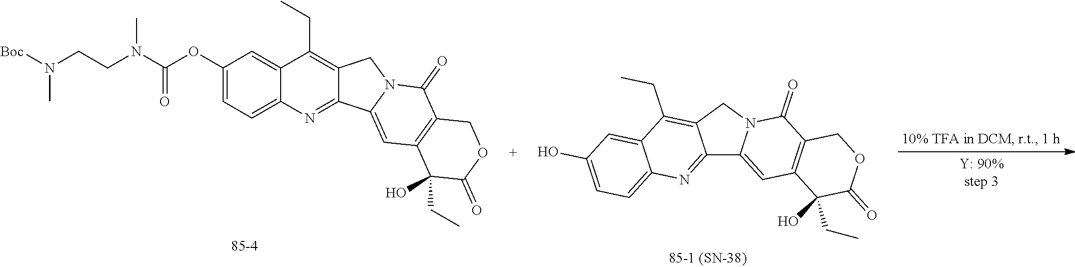 [SMILES: CCc1c2c(nc3ccc(OC(=O)N(C)CCN(C)C(=O)OC(C)(C)C)cc13)-c1cc3c(c(=O)n1C2)COC(=O)[C@]3(O)CC ; CCc1c2c(nc3ccc(O)cc13)-c1cc3c(c(=O)n1C2)COC(=O)[C@]3(O)CC]

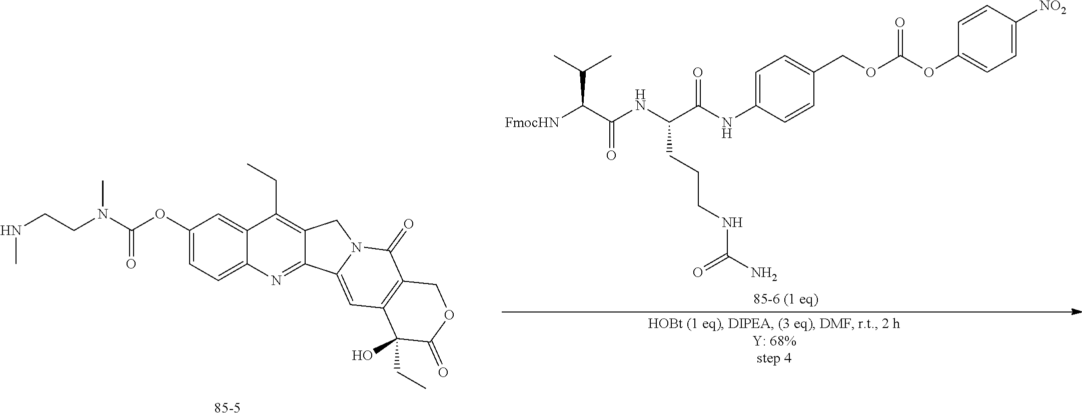

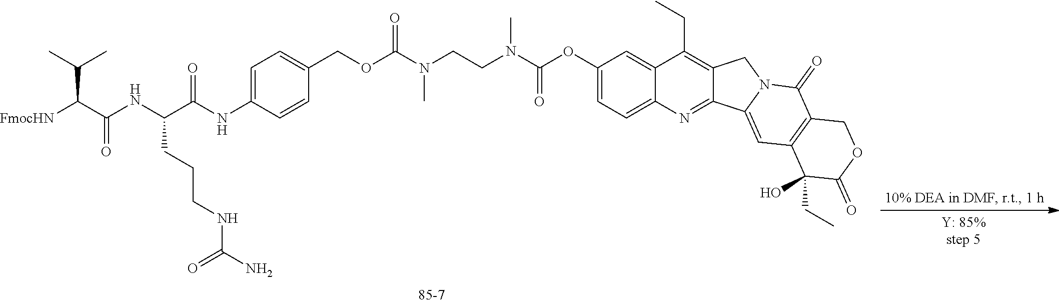

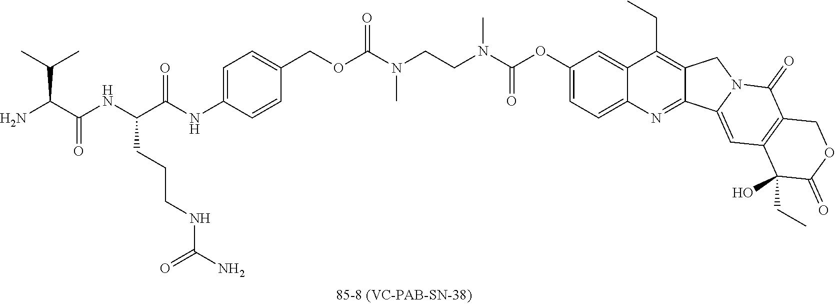 [SMILES: CCc1c2c(nc3ccc(OC(=O)N(C)CCN(C)C(=O)OCc4ccc(NC(=O)[C@H](CCCNC(N)=O)NC(=O)[C@@H](N)C(C)C)cc4)cc13)-c1cc3c(c(=O)n1C2)COC(=O)[C@]3(O)CC]

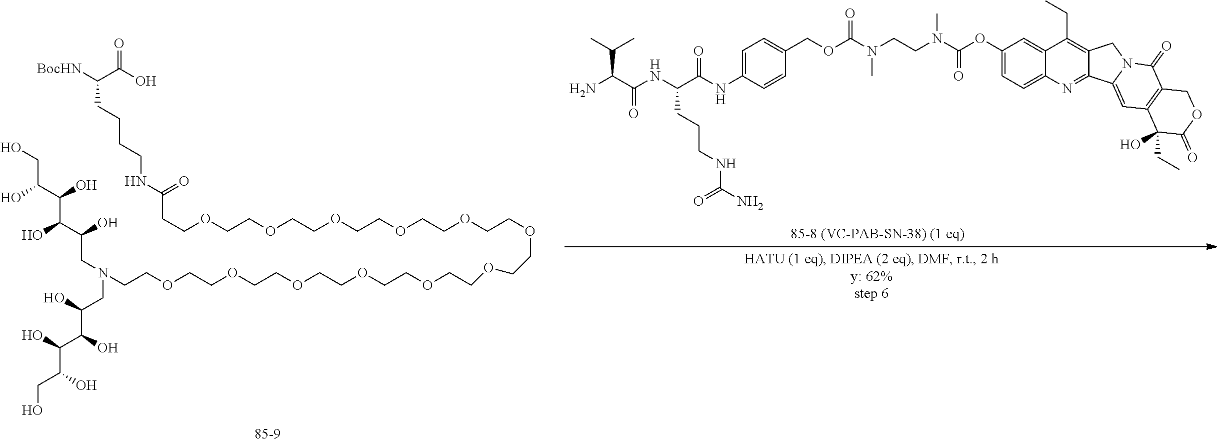 [SMILES: CC(C)(C)OC(=O)N[C@@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)O ; CCc1c2c(nc3ccc(OC(=O)N(C)CCN(C)C(=O)OCc4ccc(NC(=O)[C@H](CCCNC(N)=O)NC(=O)[C@@H](N)C(C)C)cc4)cc13)-c1cc3c(c(=O)n1C2)COC(=O)[C@]3(O)CC]

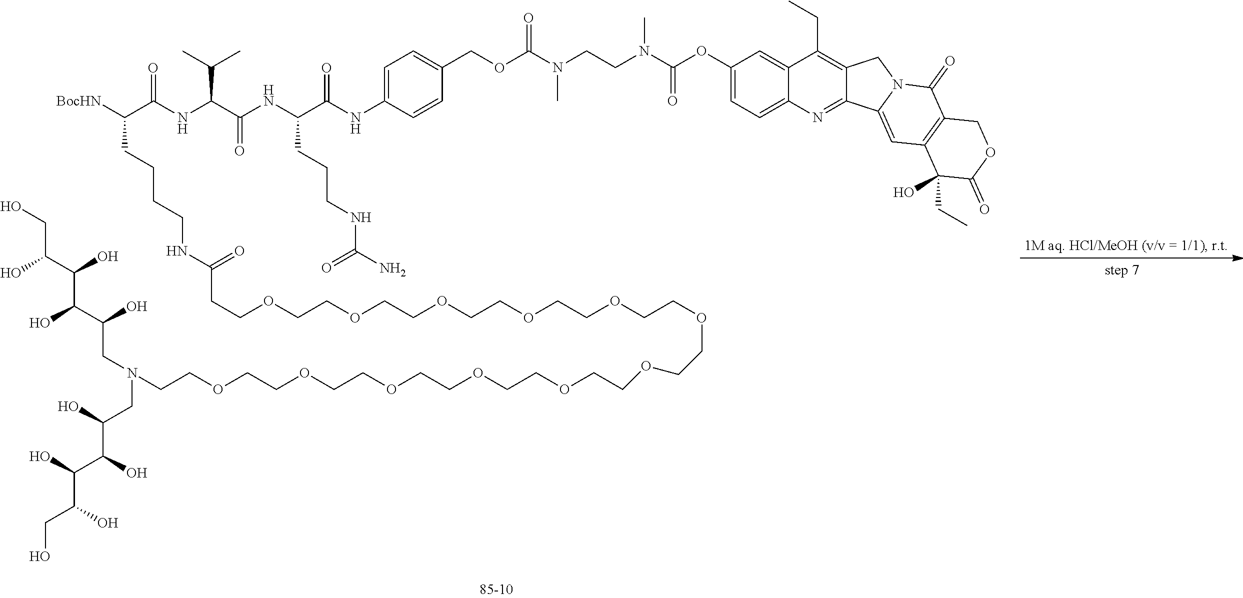 [SMILES: CCc1c2c(nc3ccc(OC(=O)N(C)CCN(C)C(=O)OCc4ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc4)cc13)-c1cc3c(c(=O)n1C2)COC(=O)[C@]3(O)CC]

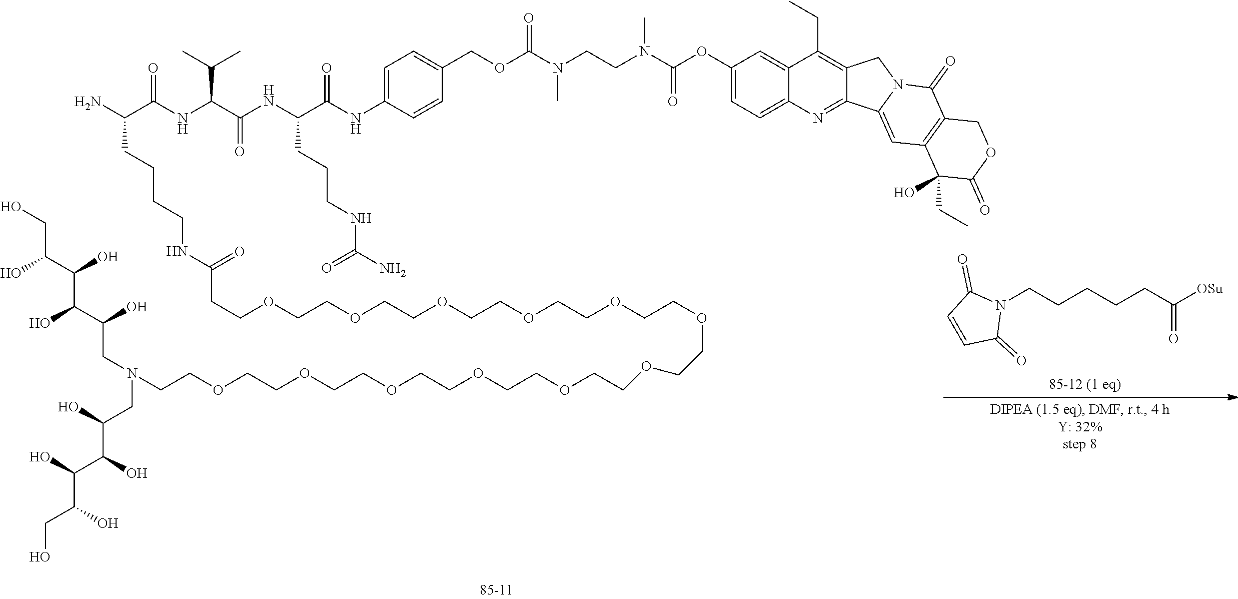

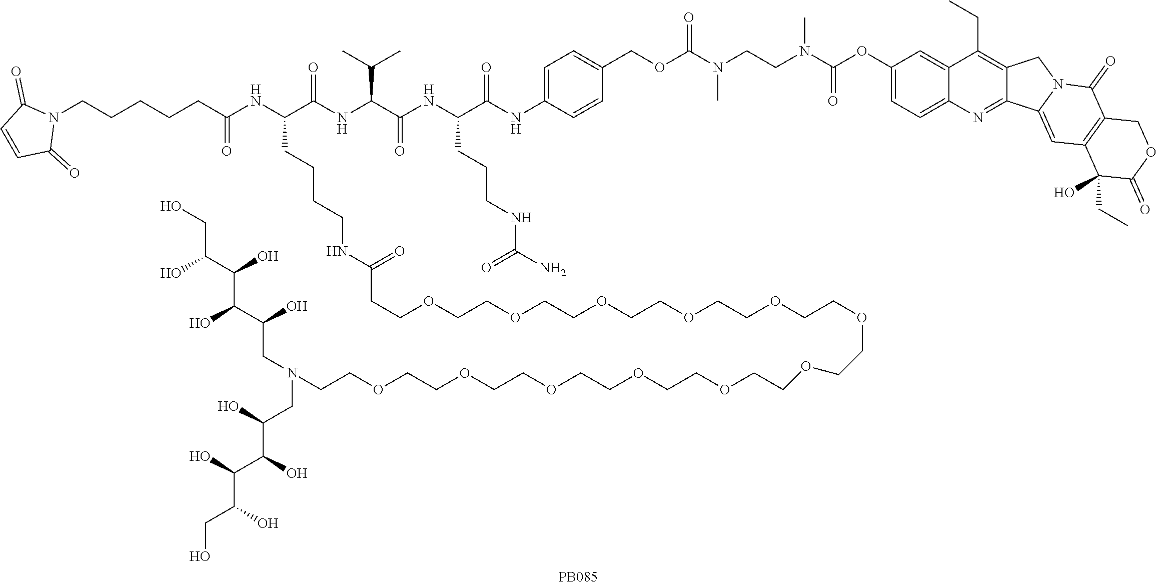

A Drug-Linker containing a PEG unit and a cleavable linker attached to SN-38 (PB085) was prepared as follows:

### Step 1

A pale yellow mixture of compound 85-1 ((19S)-10,19-diethyl-7,19-dihydroxy-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaene-14,18-dione (SN-38) (85-1, 460 mg, 1.173 mmol)) and DIPEA (302.76 mg, 2.347 mmol) in anhydrous DMF (4 mL) was stirred at room temperature, and a solution of Bis(4-nitrophenyl) carbonate (PNPC, 356.73 mg, 1.173 mmol) in anhydrous DMF (2 mL) was added dropwise over 10 min. Upon the addition, the pale yellow mixture turned into light yellow mixture, and the materials dissolved slowly as the reaction proceeded. After addition, the resulting yellow clear solution was stirred at room temperature for another 30 min and then monitored by LCMS. The spectra indicated the complete consumption of starting material SN-38, the desired phenol-activated product was formed as major new peak, along with a side product alcohol-activated carbonate. The reaction solution was used directly for the next step. ESI m/z=558.2 (M+H)⁺.

### Step 2

The DMF solution of crude activated carbonate (mix of 85-2A, 85-2B, 85-2C) from last step was treated with compound 85-3 (tert-butyl N-methyl-N-[2-(methylamino)ethyl]carbamate (85-3, 264.63 mg, 1.408 mmol)) and DIPEA (302.63 mg, 2.346 mmol). After the reaction solution was stirred for 1 h, LCMS indicated complete conversion. The desired product 85-4 and SN-38 were both detected. The reaction solution was purified by reverse phase flash chromatography (0.01% TFA) to give desired product 85-4 ((19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl N-(2-{[(tert-butoxy)carbonyl](methyl)amino}ethyl)-N-methylcarbamate (85-4, 467 mg, 0.771 mmol, 65.70%)) as a pale yellow solid. ESI m/z: 607.3 (M+H)⁺. ¹H NMR (400 MHz, DMSO-d₆) δ 10.10-10.01 (m, 1H), 8.18 (d, J=9.2 Hz, 1H), 8.10 (d, J=7.2 Hz, 1H), 7.93-7.86 (m, 3H), 7.73 (t, J=8.0 Hz, 2H), 7.62-7.52 (m, 3H), 7.43-7.26 (m, 8H), 6.52 (s, 1H), 5.97 (s, 1H), 5.45-5.34 (m, 5H), 5.09-5.00 (m, 2H), 4.44-4.40 (m, 1H), 4.32-4.22 (m, 3H), 3.93 (t, J=7.6 Hz, 1H), 3.65-3.50 (m, 7H), 3.19-3.12 (m, 2H), 3.04-2.89 (m, 7H), 2.02-1.914 (m, 1H), 1.91-1.74 (m, 2H), 1.74-1.52 (m, 2H), 1.52-1.36 (m, 2H), 1.36-1.26 (m, 2H), 0.91-0.84 (m, 9H) ppm.

### Step 3

The solution of compound 85-4 ((19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl N-(2-{[(tert-butoxy)carbonyl](methyl)amino}ethyl)-N-methylcarbamate (85-4, 600 mg, 0.990 mmol)) in DCM (1.8 mL) was stirred at room temperature, then TFA (0.2 mL, 2.693 mmol) was added. The resulting solution was stirred for 1 h until LCMS showed that the reaction was completed. All solvent and TFA were evaporated off with a rotary evaporator, then the residue was purified by reverse phase flash chromatography (0.01% TFA) to yield the expected fractions, which were lyophilized to give a TFA salt of product 85-5 ((19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl N-methyl-N-[2-(methylamino)ethyl]carbamate (85-5, 550 mg, 0.889 mmol, 89.75%)) as a pale yellow solid. ESI m/z: 254.3 (M/2+H)⁺, 507.3 (M+H)⁺. ¹H NMR (400 MHz, DMSO-d₆) δ 9.25 (s, 1H), 9.16 (s, 1H), 8.19 (d, J=9.2 Hz, 1H), 8.13 (s, 1H), 7.84-7.80 (m, 1H), 7.35 (s, 1H), 5.45 (s, 2H), 5.35 (s, 2H), 3.84-3.82 (m, 1H), 3.67-3.64 (m, 1H), 3.25-3.17 (m, 6H), 3.01 (s, 1H), 2.64-2.57 (m, 3H), 1.92-1.84 (m, 2H), 1.31 (t, J=7.2 Hz, 3H), 0.89 (t, J=7.2 Hz, 3H) ppm. One proton of carboxyl group on TFA was revealed.

### Step 4

A yellow solution of HOBt (78.39 mg, 0.581 mmol) and compound 85-6 ({4-[(2S)-5-(carbamoylamino)-2-[(2S)-2-({[(9H-fluoren-9-yl)methoxy]carbonyl}amino)-3-methylbutanamido]pentanamido]phenyl}methyl 4-nitrophenyl carbonate (85-6, 444.77 mg, 0.581 mmol)) in anhydrous DMF (2 mL) was stirred at room temperature, then a solution of DIPEA (224.71 mg, 1.742 mmol) in anhydrous DMF (2 mL) was added dropwise by syringe. The color of the reaction solution turned to brown upon the addition of base. After the addition, the resulting brown solution was stirred for another 1 h until all starting amine was consumed (monitored by LCMS). The reaction solution was purified directly by reverse phase flash chromatography (0.01% TFA) to yield compound 85-7 ({4-[(2S)-5-(carbamoylamino)-2-[(2S)-2-({[(9H-fluoren-9-yl)methoxy]carbonyl}amino)-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-7, 450 mg, 0.397 mmol, 68.33%)) as a yellow solid. ESI m/z: 568.4 (M/2+H)⁺. ¹H NMR (400 MHz, DMSO-d₆) δ 10.10-10.01 (m, 1H), 8.18 (d, J=9.2 Hz, 1H), 8.10 (d, J=7.2 Hz, 1H), 7.93-7.86 (m, 3H), 7.73 (t, J=8.0 Hz, 2H), 7.62-7.52 (m, 3H), 7.43-7.26 (m, 8H), 6.52 (s, 1H), 5.97 (s, 1H), 5.45-5.34 (m, 5H), 5.09-5.00 (m, 2H), 4.44-4.40 (m, 1H), 4.32-4.22 (m, 3H), 3.93 (t, J=7.6 Hz, 1H), 3.65-3.50 (m, 7H), 3.19-3.12 (m, 2H), 3.04-2.89 (m, 7H), 2.02-1.914 (m, 1H), 1.91-1.74 (m, 2H), 1.74-1.52 (m, 2H), 1.52-1.36 (m, 2H), 1.36-1.26 (m, 2H), 0.91-0.84 (m, 9H) ppm.

### Step 5

To a yellow solution of compound 85-7 ({4-[(2S)-5-(carbamoylamino)-2-[(2S)-2-({[(9H-fluoren-9-yl)methoxy]carbonyl}amino)-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-7, 450 mg, 0.397 mmol)) in anhydrous DMF (1.8 mL) was added diethyl amine (0.2 mL, 1.941 mmol). The solution was stirred for another 1 h until all starting material was consumed (monitored by LCMS). Then the reaction solution was evaporated with a rotary evaporator to remove most of the diethyl amine, and the residue was purified by reverse phase flash chromatography (0.01% TFA) to yield compound 85-8, VC-PAB-SN-38 TFA salt ({4-[(2S)-2-[(2S)-2-amino-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-8, VC-PAB-SN-38 TFA salt, 310 mg, 0.340 mmol, 85.62%)), as a yellow solid. ESI m/z: 456.9 (M/2+H)⁺, 912.4 (M+H)⁺. ¹H NMR (400 MHz, DMSO-d₆) δ 10.18 (s, 0.6H), 10.14 (s, 0.4H), 8.67 (d, J=8.0 Hz, 1H), 8.18 (d, J=7.6 Hz, 1H), 8.08 (brs, 3H), 7.94 (d J=7.6 Hz, 1H), 7.58-7.50 (m, 3H), 7.34-7.27 (m, 3H), 6.54 (s, 1H), 6.04 (t, J=5.6 Hz, 1H), 5.53-5.34 (m, 6H), 5.06-5.00 (m, 2H), 4.55-4.45 (m, 1H), 3.70-3.60 (m, 2H), 3.60-3.50 (m, 1H), 3.50-3.46 (m, 2H), 3.21-3.12 (m, 3H), 3.05-2.89 (m, 8H), 1.93-1.82 (m, 2H), 1.77-1.55 (m, 2H), 1.55-1.38 (m, 2H), 1.38-1.22 (m, 3H), 0.95-0.87 (m, 9H) ppm. One proton of carboxyl group on TFA overlapped with amino group at 8.08 ppm.

### Step 6

A solution of compound 85-8 ({4-[(2S)-2-[(2S)-2-amino-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-8, VC-PAB-SN-38, 240 mg, 0.263 mmol)), compound 85-9 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (85-9, 308.95 mg, 0.263 mmol)) and DIPEA (67.89 mg, 0.526 mmol) in anhydrous DMF (3 mL) was stirred at room temperature for 5 min, then a solution of HATU (100.06 mg, 0.263 mmol) in anhydrous DMF (3 mL) was added dropwise by syringe. The resulting yellow solution was stirred for another 2 h until LCMS indicated complete reaction. The reaction solution was purified directly by reverse phase liquid chromatography (0.01% TFA) to yield compound 85-10 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-10, 340 mg, 0.164 mmol, 62.48%)) as a pale yellow solid. ESI m/z: 690.3 (M/3+H)⁺. ¹H NMR (400 MHz, DMSO-d₆) δ 10.03 (s, 1H), 8.20-8.10 (m, 3H), 7.94-7.90 (m, 1H), 7.80 (t, J=13.2 Hz, 1H), 7.59-7.53 (m, 4H), 7.34-7.27 (m, 3H), 7.01 (d, J=8.0 Hz, 1H), 6.53 (s, 1H), 5.99 (t, J=5.2 Hz, 1H), 5.45-5.35 (m, 7H), 5.05-5.00 (m, 2H), 4.84-4.37 (m, 8H), 4.25 (t, J=7.6 Hz, 1H), 4.04-3.96 (m, 2H), 3.92-3.85 (m, 1H), 3.80-3.77 (m, 2H), 3.70-3.67 (m, 3H), 3.63-3.53 (m, 18H), 3.50-3.40 (m, 36H), 3.36-3.27 (m, 11H), 3.09-3.12 (m, 3H), 3.04-2.89 (m, 9H), 2.29 (t, J=6.0 Hz, 2H), 1.99-1.83 (m, 3H), 1.69-1.53 (m, 3H), 1.53-1.26 (m, 19H), 0.91-0.81 (m, 9H) ppm. One proton of carboxyl group on TFA appeared between 8.20-8.10 ppm.

### Step 7

To a solution of compound 85-10 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-10, 100 mg, 0.048 mmol)) in methanol (3 mL) was added 2M HCl in methanol (1 mL) slowly with stirring under room temperature. Upon addition, the clear solution became bright yellow. The solution was kept stirring for 2 h until LCMS indicated full deprotection. Then the solution was cooled to −20° C. and neutralized with DIPEA; as a result, the yellow reaction solution turned into colorless solution. Organic solvent was then evaporated off with rotary evaporator below 30° C. to yield a pale yellow residue, which was dissolved in water and purified by reverse phase flash chromatography (0.01% TFA) to yield the desired fractions (pH 3-4, LCMS showed the purity of fractions around 60%-58%). After lyophilization, a yellow solid (˜100 mg) was obtained with a purity of 63%-64%. Then the solid was purified again by reverse phase flash chromatography (neutral eluent) to yield the desired fractions (pH 6-7) with a purity of around 48%-51%. The collected fractions were lyophilized to yield compound 85-11 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-11, 80 mg, 0.041 mmol, 84.07%)) as pale yellow solid. The product was used directly in next step as impure material. ESI m/z: 657.0 (M/3+H)⁺, 985.2 (M/2+H)⁺.

### Step 8

To a 50 mL round-bottomed flask was added compound 85-11 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (85-11, 80 mg crude with purity 45%, 0.041 mmol)), compound 85-12 (2,5-dioxopyrrolidin-1-yl 6-(2,5-dioxo-2,5-dihydro-1H-pyrrol-1-yl)hexanoate (85-12, 12.52 mg, 0.041 mmol)) and anhydrous DMF (2 mL). The solution was stirred at room temperature and a solution of DIPEA (7.87 mg, 0.061 mmol) in anhydrous DMF (2 mL) was added dropwise by syringe over 5 min. After addition, the solution was stirred for another 4 h until LCMS indicated all starting amine was consumed. Then the completed reaction solution was neutralized with TFA and purified directly by Prep-HPLC (0.01% TFA) to yield the desired fractions, which were freeze-dried to yield a TFA salt of compound PB085 ({4-[(2S)-5-(carbamoylamino)-2-[(2S)-2-[(2S)-2-[6-(2,5-dioxo-2,5-dihydro-1H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0\hat{ }{4,9}0.0\hat{ }{15,20}]henicosa-1(21),2,4,6,8,10,15(20)-heptaen-7-yl]oxy}carbonyl)(methyl)amino]ethyl}-N-methylcarbamate (PB085, 13 mg, 0.006 mmol, yield 32% calculated on staring amine content)) as a white solid. ESI m/z: 1081.3 (M/2+H)⁺, 721.3 (M/3+H)⁺, 541.5 (M/4+H)⁺, retention time 5.819 min (HPLC). ¹H NMR (400 MHz, DMSO-d6) δ 10.00 (s, 0.6H), 9.98 (s, 0.4H), 8.20-8.08 (m, 3H), 7.94 (d, J=8.0 Hz, 2H), 7.79 (t, J=5.6 Hz, 1H), 7.66-7.52 (m, 4H), 7.34-7.26 (m, 3H), 6.99 (s, 2H), 6.53 (s, 1H), 5.98 (t, J=5.6 Hz, 1H), 5.45-5.41 (m, 5H), 5.35 (s, 2H), 5.05-4.99 (m, 2H), 4.82-4.73 (m, 2H), 4.59-4.49 (m, 4H), 4.49-4.32 (m, 3H), 4.27-4.15 (m, 2H), 4.04-3.95 (m, 2H), 3.82-3.75 (m, 2H), 3.69-3.44 (m, 65H), 3.19-3.12 (m, 3H), 3.03-2.89 (m, 11H), 2.29 (t, J=6.4 Hz, 2H), 2.14-2.08 (m, 2H), 2.02-1.84 (m, 3H), 1.72-1.60 (m, 3H), 1.55-1.15 (m, 18H), 0.90-0.80 (m, 9H) ppm. One proton of carboxyl group on TFA was revealed between 8.20-8.08 ppm.

Drug linker PB085 can be used to make a conjugate such as PA085.

### Example 17: Preparation of the Drug-Linker (PB086) Containing a PEG Unit and a Cleavable Linker Attached to Exatecan

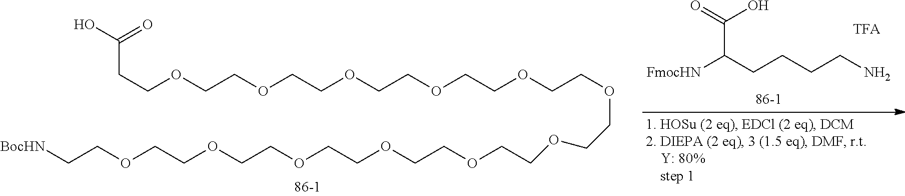

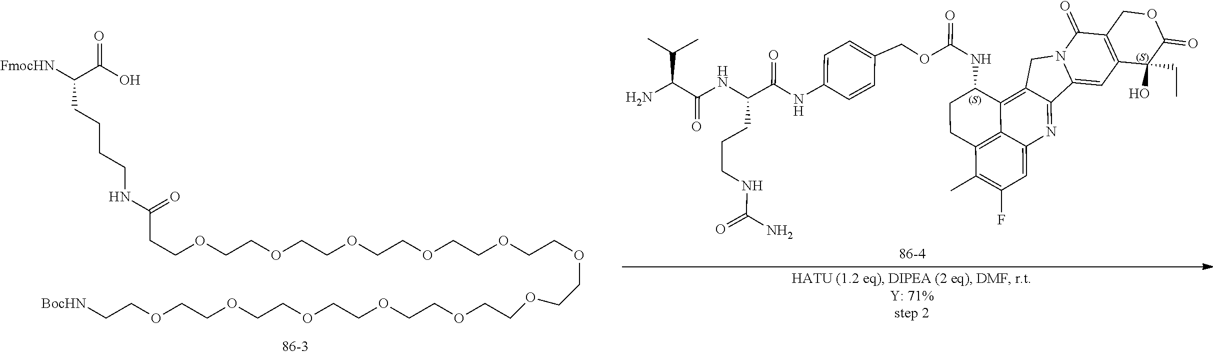

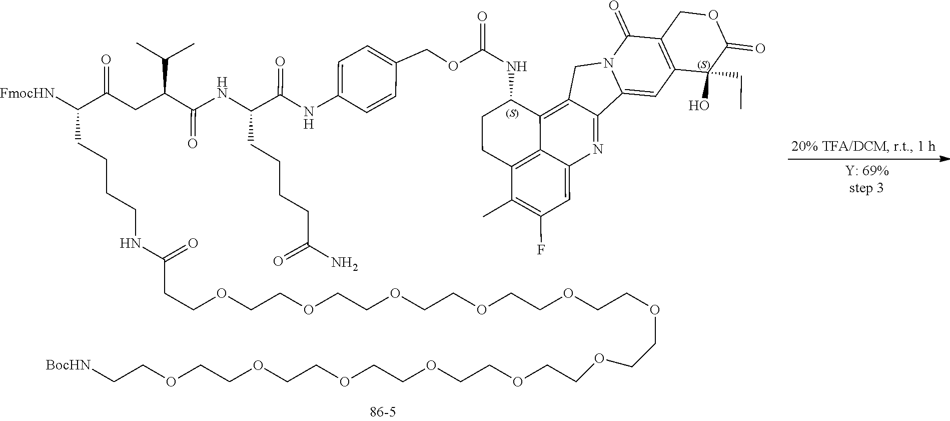

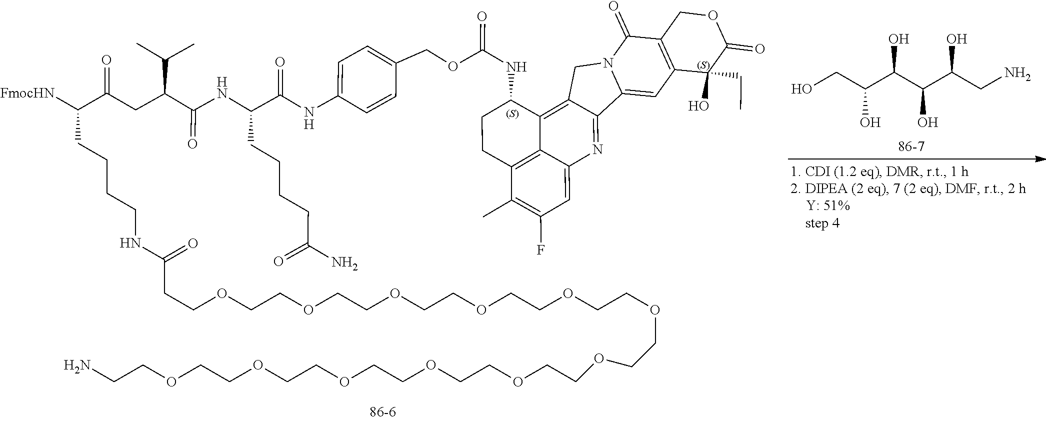

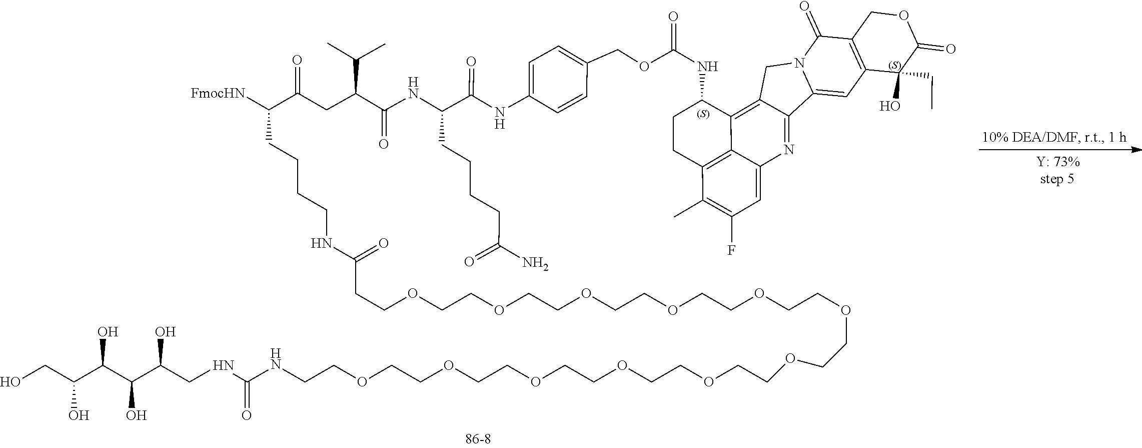

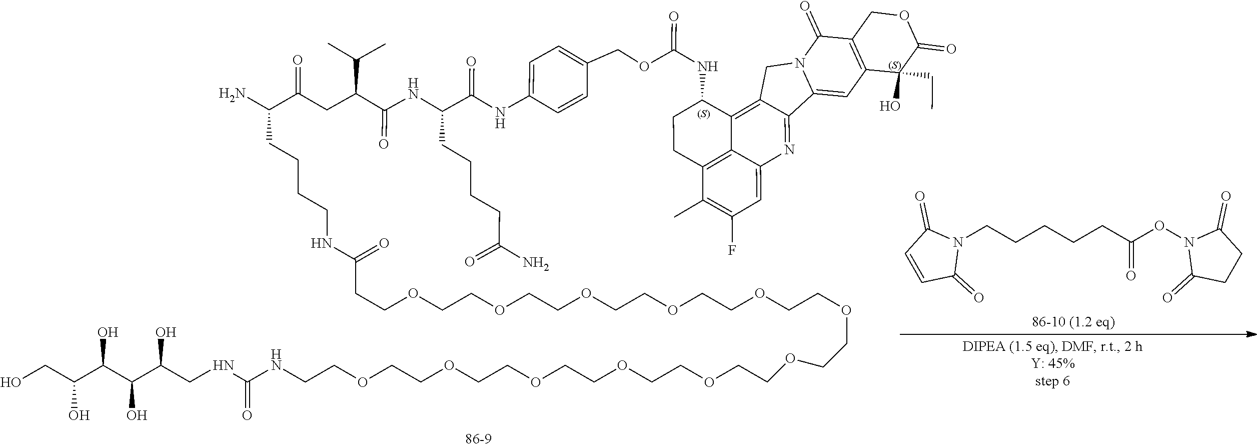

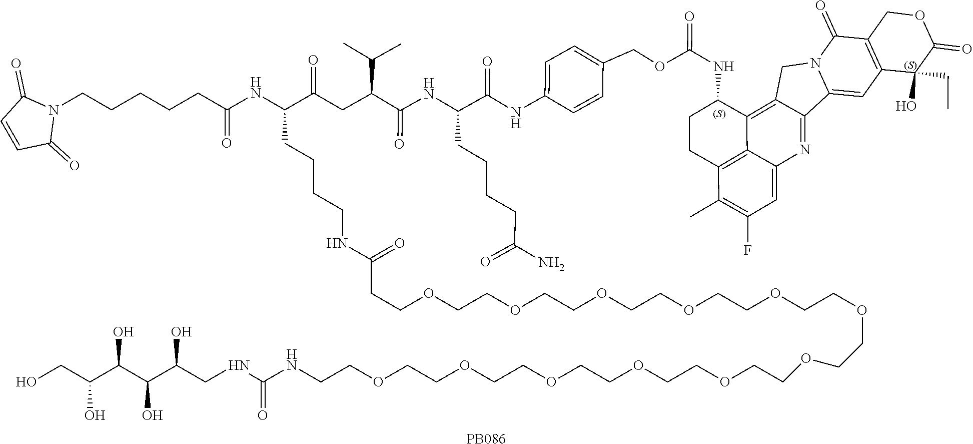

A Drug-Linker containing a PEG unit and a cleavable linker attached to exatecan (PB086) was prepared as follows:

### Step 1

A solution of compound 86-1 (1-{[(tert-butoxy)carbonyl]amino}-3,6,9,12,15,18,21,24,27,30,33,36-dodecaoxanonatriacontan-39-oic acid (86-1, 500 mg, 0.697 mmol)) and HOSu (160.39 mg, 1.395 mmol) in anhydrous DCM (14 mL) was stirred at room temperature for 5 min, then EDCI (267.36 mg, 1.395 mmol) was added. The resulting solution was stirred for another 1 h, then diluted with more DCM (20 mL) and washed with water (20 mL). The organic layer was collected, and the water layer was extracted with more DCM (20 mL). The combined DCM layer was dried over sodium sulfate, then filter and concentrated to yield crude 2,5-dioxopyrrolidin-1-yl 2,2-dimethyl-4-oxo-3,8,11,14,17,20,23,26,29,32,35,38,41-tridecaoxa-5-azatetratetracontan-44-oate (800 mg, quantative yield) as colorless oil. ESI m/z: 715.5 (M−100+H)⁺; 837.5 (M+Na)⁺.

The activated ester (800 mg crude, 0.697 mmol) was dissolved in anhydrous DMF (4 mL), then compound 86-2 ((2S)-6-amino-2-({[(9H-fluoren-9-yl)methoxy]carbonyl}amino)hexanoic acid, TFA salt (86-2, 503.93 mg, 1.045 mmol)) and DIPEA (179.83 mg, 1.394 mmol) were added. The reaction solution was stirred at room temperature for 1 h until all activated ester was consumed (monitored by LCMS). Then the resulting solution was purified directly by reverse phase flash chromatography (0.01% TFA) to yield compound 86-3 ((2S)-6-(1-{1[(tert-butoxy)carbonyl]amino}-3,6,9,12,15,18,21,24,27,30,33,36-dodecaoxanonatriacontan-39-amido)-2-({1[(9H-fluoren-9-yl)methoxy]carbonyl}amino)hexanoic acid (86-3, 600 mg, 0.562 mmol, 80.58%)) as pale yellow oil. ESI m/z: 484.9 ((M−100)/2+H)⁺, 968.7 (M−100+H)⁺.

¹H NMR (400 MHz, DMSO-d6) δ 12.64 (s, 1H), 7.90 (d, J=7.2 Hz, 2H), 7.82 (t, J=5.6 Hz, 1H), 7.73 (d, J=7.2 Hz, 2H), 7.62 (d, J=8.0 Hz, 1H), 7.45-7.40 (m, 2H), 7.36-7.31 (m, 2H), 7.76 (t, J=5.6 Hz, 1H), 4.29-4.21 (m, 3H), 3.94-3.88 (m, 1H), 3.68-3.56 (m, 4H), 3.51-3.47 (m, 44H), 3.07-3.00 (m, 4H), 2.29 (t, J=6.4 Hz, 2H), 1.73-1.60 (m, 2H), 1.52-1.29 (m, 13H) ppm.

### Step 2

A solution of compound 86-3 ((2S)-6-(1-{[(tert-butoxy)carbonyl]amino}-3,6,9,12,15,18,21,24,27,30,33,36-dodecaoxanonatriacontan-39-amido)-2-({[(9H-fluoren-9-yl)methoxy]carbonyl}amino)hexanoic acid (86-3, 600 mg, 0.562 mmol)), HATU (256.43 mg, 0.674 mmol) and DIPEA (145.00 mg, 1.124 mmol) in anhydrous DMF (4 mL) was stirred at room temperature for 5 min, then compound 86-4 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (86-4, 471.91 mg, 0.562 mmol)) was added. The resulting solution was stirred for another 1 h until LCMS indicated complete reaction. Then the solution was purified directly by reverse phase flash chromatography (0.01% TFA) to yield compound 86-5 (tert-butyl N-(38-{[(5S)-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18,21,24,27,30,33,36-dodecaoxaoctatriacontan-1-yl)carbamate (86-5, 750 mg, 0.397 mmol, 70.60%)) as an off-white solid. ESI m/z: 597.9 ((M−100)/3+H)⁺, 896.1 ((M−100)/2+H)⁺, 946.7 (M/2+H)⁺.

### Step 3

To the solution of compound 86-5 (tert-butyl N-(38-{[(5S)-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18,21,24,27,30,33,36-dodecaoxaoctatriacontan-1-yl)carbamate (86-5, 700 mg, 0.370 mmol)) in DCM (8 mL) TFA (2 mL, 26.925 mmol) was added slowly. The resulting yellow solution was stirred at room temperature for 1 h to complete. TFA and solvent were evaporated with a rotary evaporator, and the residue was purified by reverse phase flash chromatography (0.01% TFA) to yield the expected fractions, which were lyophilized to yield product 86-6 ((9H-fluoren-9-yl)methyl N-[(1S)-5-(1-amino-3,6,9,12,15,18,21,24,27,30,33,36-dodecaoxanonatriacontan-39-amido)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (86-6, 460 mg, 0.257 mmol, 69.38%)) as a white solid. ESI m/z: 896.6 (M/2+H)⁺, 597.9 (M/3+H)⁺.

### Step 4

To a solution of CDI (0.029 mL, 0.234 mmol) in anhydrous DMF (4 mL) was added a solution of compound 86-6 ((9H-fluoren-9-yl)methyl N-[(1S)-5-(1-amino-3,6,9,12,15,18,21,24,27,30,33,36-dodecaoxanonatriacontan-39-amido)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (86-6, 350 mg, 0.195 mmol)) in anhydrous DMF (4 mL) dropwise by syringe over 5 min. After addition, the pale yellow solution was stirred at room temperature for another 1 h to completion (monitored by LCMS). Activated intermediate (9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[(1H-imidazole-1-carbonyl)amino]-3,6,9,12,15,18,21,24,27,30,33,36-dodecaoxanonatriacontan-39-amido}pentyl]carbamate was detected as major peak. ESI m/z: 629.3 (M/3+H)⁺, 943.3 (M/2+H)⁺.

To the reaction solution of activated intermediate (0.195 mmol) in DMF (4 mL) above was added compound 86-7 ((2R,3R,4R,5S)-6-aminohexane-1,2,3,4,5-pentol (86-7, 70.59 mg, 0.390 mmol)) and DIPEA (50.31 mg, 0.390 mmol). The resulting solution was stirred at room temperature for 2 h until all materials were consumed. Then the solution was purified directly by reverse phase flash chromatography (0.01% TFA) to yield compound 86-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]pentyl]carbamate (86-8, 200 mg, 0.100 mmol, 51.33%)) as a pale yellow solid. ESI m/z: 999.8 (M/2+H)⁺, 666.9 (M/3+H)⁺.

### Step 5

A solution of compound 86-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]pentyl]carbamate (86-8, 200 mg, 0.100 mmol)) in DMF (3.6 mL) was stirred at room temperature and diethyl amine (0.4 mL, 3.883 mmol) was added. The resulting solution was stirred for 30 min. Then diethyl amine was evaporated under vacuo, the residue in DMF was neutralized with formic acid and purified by reverse phase flash chromatography (0.01% TFA) to yield the expected product 86-9 ({4-[(2S)-2-[(2S)-2-[(2S)-2-amino-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (86-9, 130 mg, 0.073 mmol, 73.14%)) as a pale yellow solid. ESI m/z: 592.9 (M/3+H)⁺, 889.0 (M/2+H)⁺. ¹H NMR (400 MHz, DMSO-d6) δ 10.09 (s, 1H), 8.42 (d, J=8.4 Hz, 1H), 8.31 (d, J=6.8 Hz, 1H), 8.12-8.04 (m, 4H), 7.85 (t, J=5.6 Hz, 1H), 7.78 (d, J=10.8 Hz, 1H), 7.60 (d, J=7.6 Hz, 2H), 7.37 (d, J=8.8 Hz, 2H), 7.32 (s, 1H), 6.54 (brs, 1H), 6.12 (t, J=5.6 Hz, 1H), 6.04 (t, J=5.6 Hz, 1H), 5.97 (t, J=5.6 Hz, 1H), 5.54-5.45 (m, 3H), 5.40-5.23 (m, 3H), 5.09 (s, 2H), 4.44-4.38 (m, 2H), 4.30-4.27 (m, 1H), 3.89-3.81 (m, 1H), 3.69-3.54 (m, 6H), 3.54-3.45 (m, 48H), 3.26-3.13 (m, 6H), 3.13-3.05 (m, 4H), 3.05-2.98 (m, 3H), 2.98-2.89 (m, 2H), 2.37 (s, 3H), 2.29 (t, J=6.4 Hz, 2H), 2.24-2.10 (m, 2H), 2.04-1.93 (m, 1H), 1.93-1.82 (m, 2H), 1.72-1.54 (m, 4H), 1.45-1.23 (m, 6H), 0.92-0.86 (m, 9H) ppm. One proton of carboxyl group on TFA appeared between 8.12-8.04 ppm.

### Step 6

A solution of compound 86-9 ({4-[(2S)-2-[(2S)-2-[(2S)-2-amino-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (86-9, 60 mg, 0.034 mmol)) and compound 86-10 (2,5-dioxopyrrolidin-1-yl 6-(2,5-dioxo-2,5-dihydro-1H-pyrrol-1-yl)hexanoate (86-10, 12.50 mg, 0.041 mmol)) in anhydrous DMF (1.5 mL) was stirred at room temperature, then DIPEA (6.54 mg, 0.051 mmol) was added. The resulting solution was stirred for another 2 h until LCMS indicated all starting amine was consumed. The resulting solution was neutralized with formic acid, then purified by Prep-HPLC (0.01% TFA) to yield the desired fractions, which were freeze-dried to yield PB086 ({4-[(2S)-5-(carbamoylamino)-2-[(2S)-2-[(2S)-2-[6-(2,5-dioxo-2,5-dihydro-1H-pyrrol-1-yl)hexanamido]-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB086, 30 mg, 0.015 mmol, 45.09%)) as a white solid. ESI m/z: 985.1 (M/2+H)⁺, 657.3 (M/3+H)⁺, 493.3 (M/4+H)⁺, retention time 6.424 min (HPLC). ¹H NMR (400 MHz, DMSO-d6) δ 10.03 (s, 1H), 8.11 (d, J=7.2 Hz, 1H), 8.06 (d, J=8.8 Hz, 1H), 7.96 (d, J=8.0 Hz, 1H), 7.82-7.76 (m, 2H), 7.66 (d, J=8.8 Hz, 1H), 7.60 (d, J=8.4 Hz, 2H), 7.36 (d, J=8.4 Hz, 2H), 7.32 (s, 1H), 6.99 (s, 2H), 6.50 (brs, 1H), 6.11 (t, J=5.6 Hz, 1H), 6.00-5.94 (m, 2H), 5.45-5.38 (m, 3H), 5.35-5.24 (m, 3H), 5.08 (s, 2H), 4.40-4.17 (m, 4H), 3.59-3.45 (m, 54H), 3.24-3.16 (m, 4H), 3.16-3.09 (m, 4H), 3.04-2.90 (m, 7H), 2.38 (s, 3H), 2.29 (t, J=6.4 Hz, 2H), 2.23-2.13 (m, 2H), 2.13-2.06 (m, 2H), 2.01-1.96 (m, 1H), 1.96-1.81 (m, 2H), 1.73-1.54 (m, 4H), 1.48-1.29 (m, 10H), 1.24-1.14 (m, 4H), 0.90-0.81 (m, 9H) ppm.

Drug linker PB086 can be used to make a conjugate such as PA086.

### Example 18: Preparation of the Drug-Linker (PB087) Containing a PEG Unit and a Cleavable Linker Attached to Exatecan

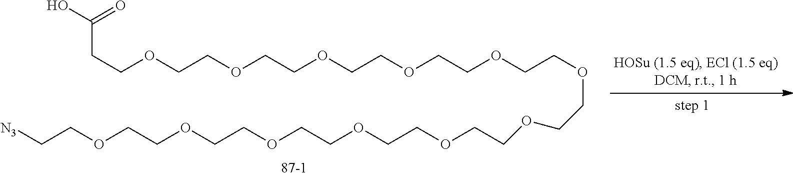 [SMILES: [N-]=[N+]=NCCOCCOCCOCCOCCOCCOCCOCCOCCOCCOCCOCCOCCC(=O)O]

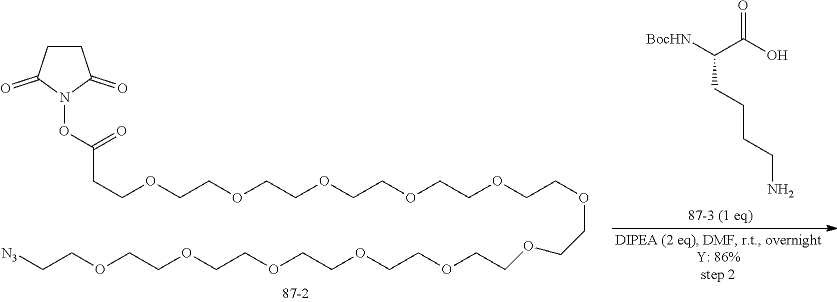 [SMILES: [N-]=[N+]=NCCOCCOCCOCCOCCOCCOCCOCCOCCOCCOCCOCCOCCC(=O)ON1C(=O)CCC1=O ; CC(C)(C)OC(=O)N[C@@H](CCCCN)C(=O)O]

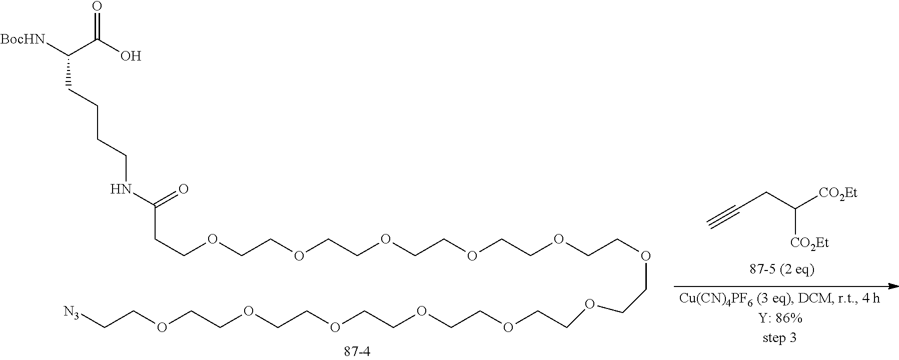 [SMILES: CC(C)(C)OC(=O)N[C@@H](CCCCNC(=O)CCOCCOCCOCCOCCOCCOCCOCCOCCOCCOCCOCCOCCN=[N+]=[N-])C(=O)O ; C#CCC(C(=O)OCC)C(=O)OCC]

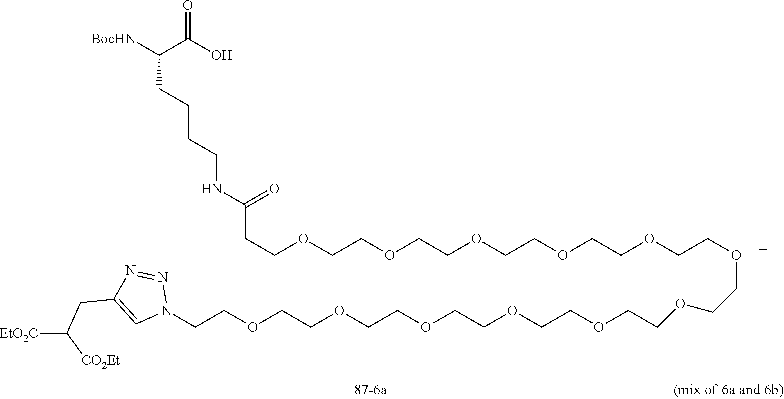 [SMILES: CCOC(=O)C(Cc1cn(CCOCCOCCOCCOCCOCCOCCOCCOCCOCCOCCOCCOCCC(=O)NCCCC[C@H](NC(=O)OC(C)(C)C)C(=O)O)nn1)C(=O)OCC]

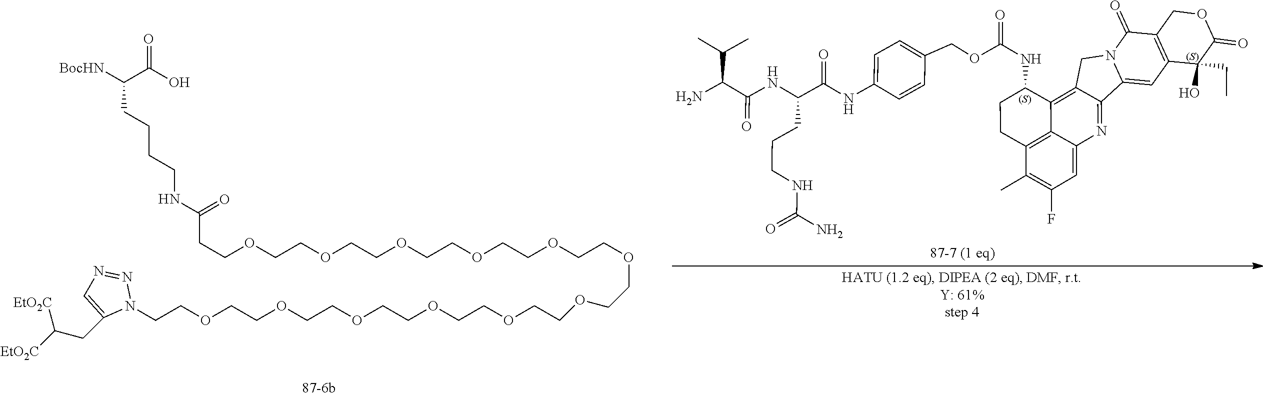

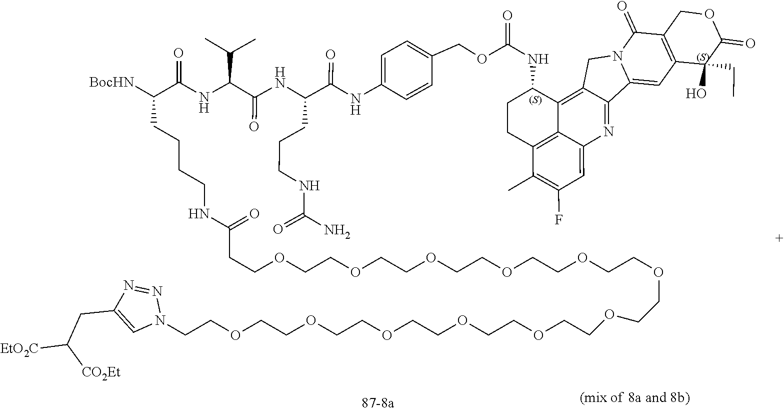

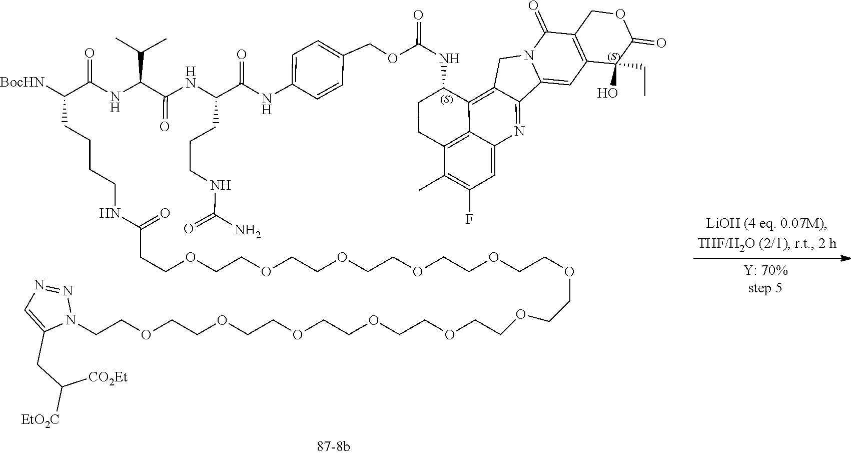

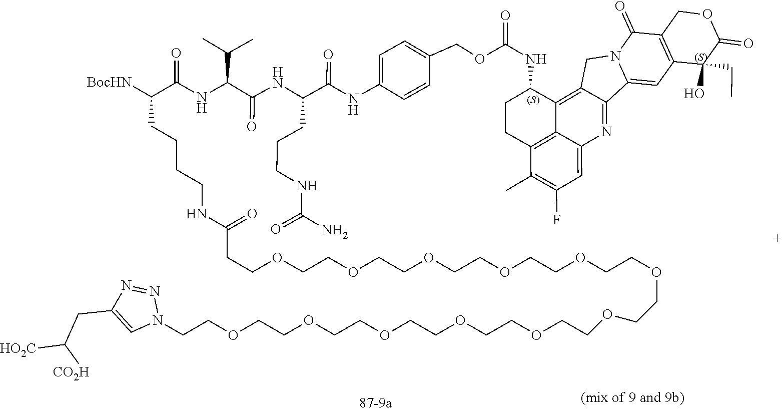

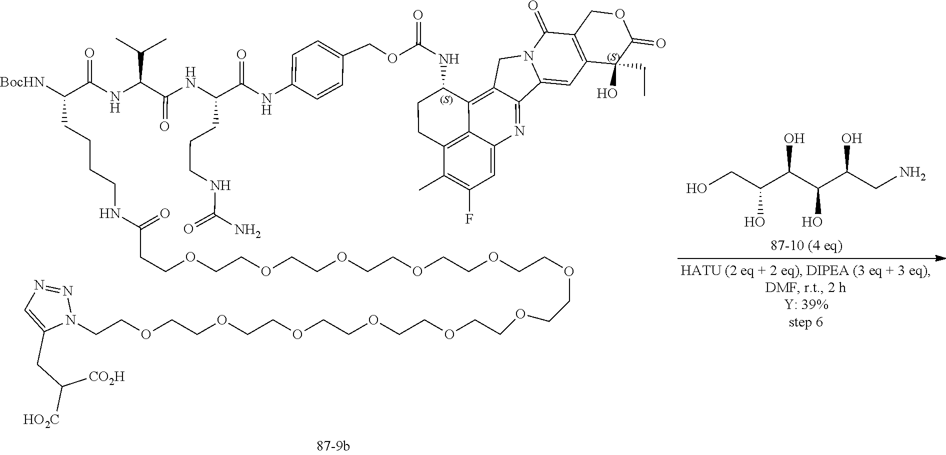

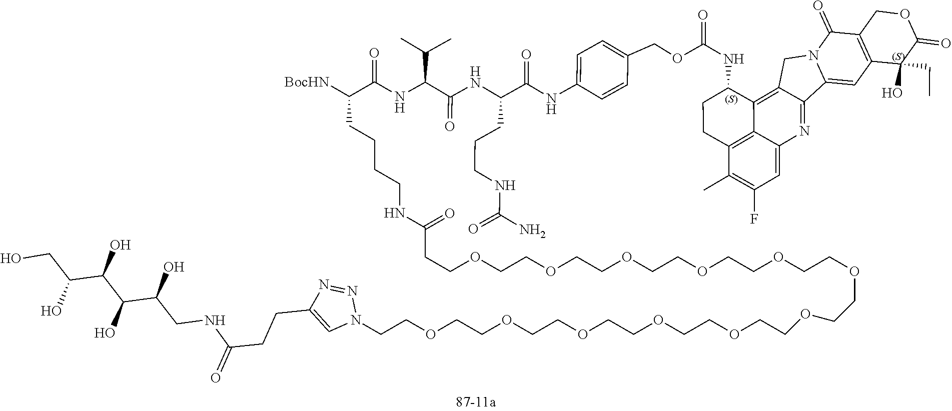

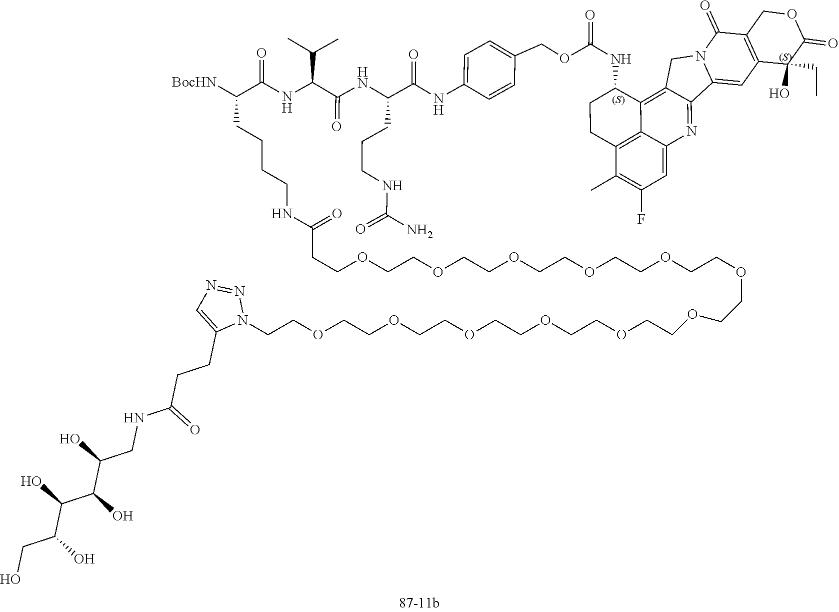

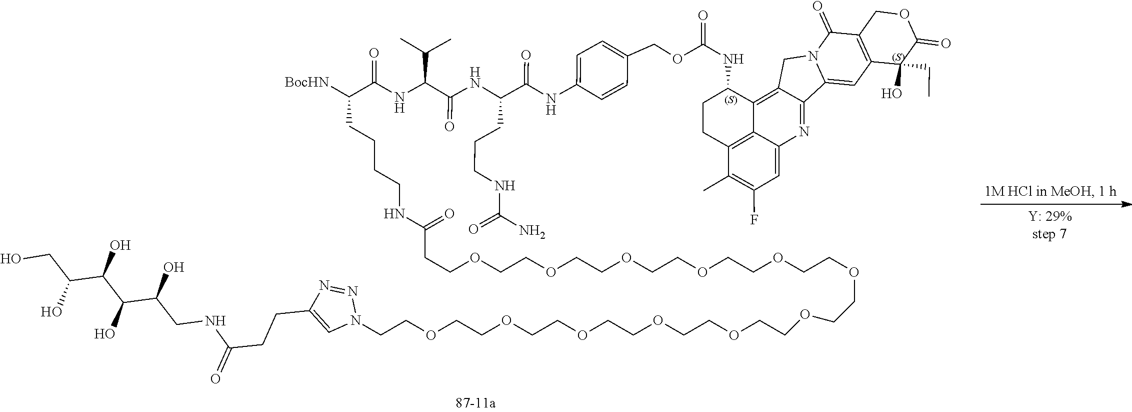

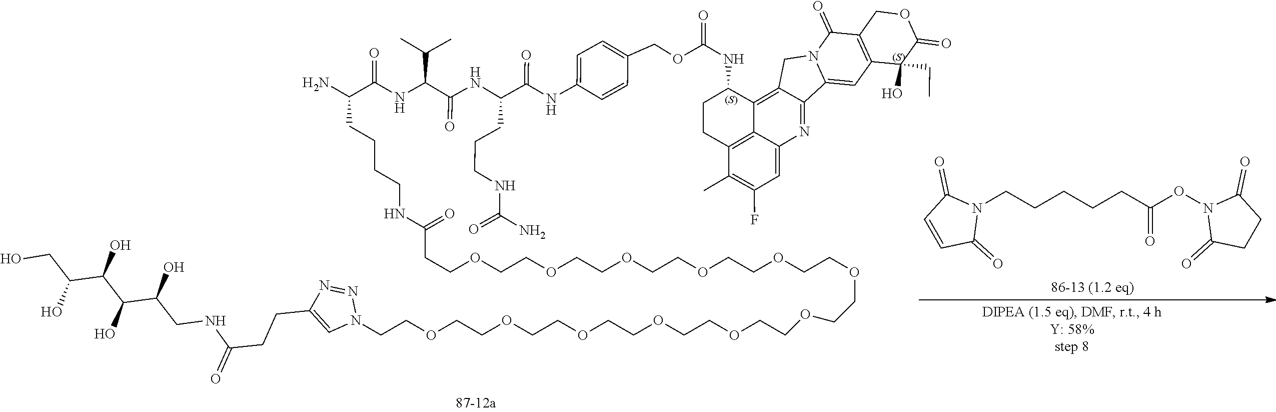

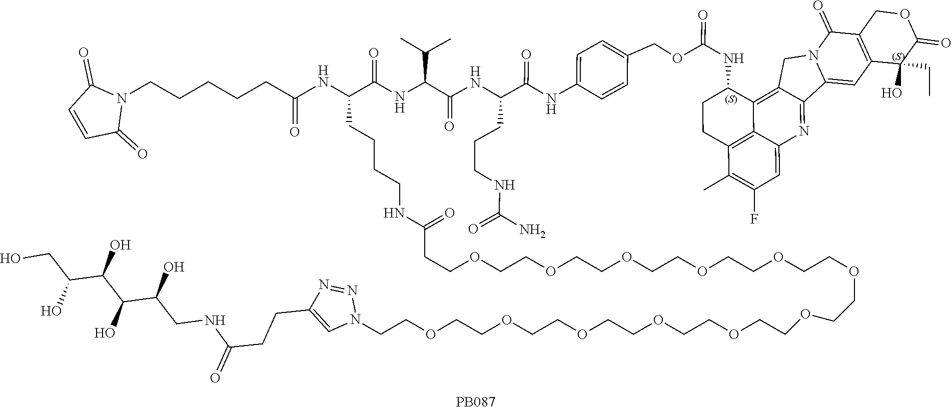

A Drug-Linker containing a PEG unit and a cleavable linker attached to exatecan (PB087) was prepared as follows:

### Steps 1 & 2

A solution of compound 87-1 (1-azido-3,6,9,12,15,18,21,24,27,30,33,36-dodecaoxanonatriacontan-39-oic acid (87-1, 300 mg, 0.466 mmol)) and HOSu (80.39 mg, 0.699 mmol) in anhydrous DCM (10 mL) was stirred at room temperature for 5 min, then EDCI (134.01 mg, 0.699 mmol) was added at room temperature. The resulting solution was stirred for another 1 h, then diluted with more DCM (20 mL) and washed with water (20 mL), the organic layer was separated and the water layer was extracted with more DCM (20 mL*2). The combined DCM layer was dried over sodium sulfate, filtered and concentrated to yield crude activated ester (345 mg, quantative yield) as a colorless oil. ESI m/z: 741.5 (M+H)⁺, 763.4 (M+Na)⁺. The ester was dissolved in anhydrous DMF (4 mL), then compound 87-3 ((2S)-6-amino-2-{[(tert-butoxy)carbonyl]amino}hexanoic acid (87-3, 114.78 mg, 0.466 mmol)) and DIPEA (120.23 mg, 0.932 mmol) were added. The resulting mixture was stirred at room temperature for overnight until all starting amine was consumed, and the mixture turned into clear pale yellow solution. Then the completed reaction solution was purified directly by reverse phase flash chromatography (0.01% TFA) to yield compound 87-4((S)-1-azido-45-((tert-butoxycarbonyl)amino)-39-oxo-3,6,9,12,15,18,21,24,27,30,33,36-dodecaoxa-40-azahexatetracontan-46-oic acid (87-4, 350 mg, 0.401 mmol, 86.13%)) as a colorless oil. ESI m/z: 386.8 ((M−100)/2+H)⁺, 872.6 (M+H)⁺.

### Step 3

A solution of compound 87-4 ((S)-1-azido-45-((tert-butoxycarbonyl)amino)-39-oxo-3,6,9,12,15,18,21,24,27,30,33,36-dodecaoxa-40-azahexatetracontan-46-oic acid (87-4, 350 mg, 0.401 mmol)) and compound 87-5 (1,3-diethyl 2-(prop-2-yn-1-yl)propanedioate (87-5, 158.94 mg, 0.803 mmol)) in DCM (8 mL) was stirred at room temperature, then Cu(CN)₄PF₆ (447.94 mg, 1.204 mmol) was added. The resulting solution was stirred for another 4 h until the reaction was completed (monitored by LCMS). The reaction solution was then concentrated and dissolved in acetonitrile, and filtered to remove the copper catalyst. The filtrate was purified by reverse phase flash chromatography (0.01% TFA) to yield a mixture (compounds 87-6a/87-6b, 350 mg, 0.327 mmol, 81.57%) of 1,4-disubstituted triazole isomer (2S)-2-{[(tert-butoxy)carbonyl]amino}-6-(1-{4-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid and 1,5-disubstituted triazole isomer (2S)-2-{[(tert-butoxy)carbonyl]amino}-6-(1-{5-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid as a white solid. ESI m/z: 485.9 ((M−100)/2+H)⁺, both regioselective isomers overlapped in LCMS.

### Step 4

A mixture of compounds 87-6a/87-6b (350 mg, 0.327 mmol of (2S)-2-{[(tert-butoxy)carbonyl]amino}-6-(1-{5-[3-ethoxy-2-(ethoxycarbonyl)-3-oxobutyl]-1H-1,2,3-triazol-1-yl}-3,6,9,12,15,18,21,24,27,30,33,36-dodecaoxanonatriacontan-39-amido)hexanoic acid and (2S)-2-{[(tert-butoxy)carbonyl]amino}-6-(1-{4-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid), HATU (149.25 mg, 0.393 mmol) and DIPEA (23.99 mg, 0.186 mmol) in anhydrous DMF (2 mL) was stirred at room temperature for 5 min, then compound 87-7 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (87-7, 275.06 mg, 0.327 mmol)) was added. The resulting solution was stirred for another 1 h until LCMS indicated complete reaction. The reaction solution was purified directly by reverse phase flash chromatography (0.01% TFA) to yield a mixture of compounds 87-8a/87-8b (380 mg, 0.201 mmol, 61.36% of 1,3-diethyl 2-{[1-(38-{[(5S)-5-{[(tert-butoxy)carbonyl]amino}-5-{1[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,11}.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioate and its regioselective isomer1,3-diethyl 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,11}.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioate) as a white solid. ESI m/z: 449.4 ((M−100)/4+H)⁺, 632.1 (M/3+H)⁺, 947.7 (M/2+H)⁺, retention time 8.205 min (HPLC). Both isomers overlapped as one peak in LCMS. ¹H NMR (400 MHz, DMSO-d6): δ 10.06 (s, 1H), 8.20 (d, J=7.2 Hz, 1H), 8.07 (d, J=8.0 Hz, 1H), 7.84-7.76 (m, 3H), 7.61 (d, J=8.4 Hz, 2H), 7.55 (d, J=8.4 Hz, 1H), 7.36 (d, J=8.8 Hz, 2H), 7.31 (s, 1H), 7.02 (d, J=8.0 Hz, 1H), 6.63-6.43 (m, 1H), 5.99 (s, 1H), 5.50-5.40 (m, 3H), 5.34-5.23 (m, 3H), 5.08 (s, 2H), 4.48-4.45 (m, 2H), 4.42-4.36 (m, 1H), 4.27-4.23 (m, 1H), 4.13-4.08 (m, 4H), 3.93-3.86 (m, 1H), 3.82 (t, J=7.6 Hz, 1H), 3.77 (t, J=4.2 Hz, 2H), 3.58-3.55 (m, 2H), 3.50-3.45 (m, 44H), 3.32-3.21 (m, 2H), 3.13 (d, J=8.0 Hz, 2H), 3.04-2.92 (m, 5H), 2.38 (s, 3H), 2.29 (d, J=6.4 Hz, 2H), 2.23-2.10 (m, 2H), 1.99-1.71 (m, 3H), 1.71-1.48 (m, 3H), 1.48-1.21 (m, 16H), 1.15 (t, J=6.8 Hz, 6H), 0.90-0.81 (m, 9H) ppm.

### Step 5

A mixture of compounds 87-8a/87-8b (1,3-diethyl 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioate (87-8a/87-8b, 380 mg, 0.201 mmol) and 1,3-diethyl 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioate) in THF (6 mL) was stirred at room temperature, then lithium hydroxide monohydrate (8.87 mg, 0.211 mmol) in water (3 mL) was added. The reaction solution turned yellow and was stirred for another 2 h to achieve complete hydrolysis. The solution was acidified with TFA to pH 6.0, then concentrated under reduced pressure to remove THF, and the residue in water was purified by reverse phase flash chromatography (0.01% TFA) to yield a mixture of compounds 87-9a/87-9b (250 mg, 0.136 mmol, 67.80%, of 1,4-disubstituted triazole isomer 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioic acid and 1,5-disubstituted triazole isomer 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioic acid) as a white solid. About 30%-44% isomer 1 at retention time 1.792 min, 40%-52% isomer 2 at retention time 1.811 min, ESI m/z=613.3 (M/3+H)⁺; 919.1 (M/2+H)⁺. 1H NMR (400 MHz, DMSO-d6): δ 10.05 (s, 1H), 8.19 (d, J=7.2 Hz, 1H), 8.07-8.03 (m, 1H), 7.82-7.76 (m, 3H), 7.61 (d, J=7.2 Hz, 2H), 7.54 (d, J=8.8 Hz, 1H), 7.37 (d, J=8.8 Hz, 2H), 7.33 (s, 0.5H), 7.32 (s, 0.5H), 7.01 (d, J=8.0 Hz, 1H), 6.54-6.53 (m, 1H), 5.99 (s, 1H), 5.46-5.39 (m, 3H), 5.34-5.23 (m, 3H), 5.09 (s, 2H), 4.48-4.46 (m, 2H), 4.43-4.37 (m, 1H), 4.28-4.23 (m, 1H), 3.91-3.86 (m, 1H), 3.78 (t, J=5.6 Hz, 1H), 3.65-3.56 (m, 4H), 3.51-3.45 (m, 46H), 3.15-2.90 (m, 8H), 2.38 (s, 3H), 2.29 (d, J=6.4 Hz, 2H), 2.23-2.11 (m, 2H), 1.99-1.82 (m, 3H), 1.72-1.49 (m, 3H), 1.49-1.22 (m, 17H), 0.90-0.81 (m, 9H) ppm.

### Step 6

To a round-bottomed flask was added a mixture of compounds 87-9a/87-9b (240 mg, 0.131 mmol, of 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioic acid and 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioic acid), followed by a solution of compound 87-10 ((2R,3R,4R,5S)-6-aminohexane-1,2,3,4,5-pentol (87-10, 94.69 mg, 0.523 mmol)) and DIPEA (50.56 mg, 0.392 mmol) in anhydrous DMF (1.5 mL). The brown solution was stirred at room temperature for 5 min, then a solution of HATU (99.35 mg, 0.261 mmol) in anhydrous DMF (1.5 mL) was added dropwise. After addition, the resulting solution was stirred for another 2 hr until LCMS indicated complete conversion. The reaction solution was purified directly by reverse phase flash chromatography (0.01% TFA) to yield a mixture of 1,4-disubstituted triazole and 1,5-disubstituted triazole, which was further purified by Prep-HPLC (0.01% TFA) to yield compound 87-11a (1,4-disubstituted isomer tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[4-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}pentyl]carbamate (87-11a, 65 mg, 0.033 mmol, 25.43%)) as a pale yellow solid, and then compound 87-11b (1,5-disubstituted isomer tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[5-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}pentyl]carbamate (87-11b, 35 mg, 0.018 mmol, 13.66%)) as a pale yellow solid. 87-11a (majority): ESI m/z: 653.0 (M/3+H)⁺, 979.2 (M/2+H)⁺. ¹H NMR: (400 MHz, DMSO-d6) δ 10.04 (s, 1H), 8.19 (d, J=6.8 Hz, 1H), 8.06 (d, J=7.6 Hz, 1H), 7.86-7.77 (m, 4H), 7.60 (d, J=8.8 Hz, 2H), 7.54 (d, J=8.4 Hz, 1H), 7.36 (d, J=8.4 Hz, 2H), 7.32 (s, 1H), 7.00 (d, J=8.0 Hz, 1H), 6.53 (s, 1H), 5.99 (s, 1H), 5.45-5.35 (m, 3H), 5.35-5.24 (m, 3H), 5.08 (s, 2H), 4.47-4.44 (m, 2H), 4.41-4.36 (m, 1H), 4.27-4.23 (m, 1H), 3.93-3.86 (m, 1H), 3.79 (t, J=4.2 Hz, 2H), 3.67-3.56 (m, 8H), 3.56-3.46 (m, 44H), 3.16-2.90 (m, 12H), 2.83 (t, J=7.6 Hz, 2H), 2.43 (t, J=8.0 Hz, 2H), 2.38 (s, 3H), 2.29 (t, J=6.4 Hz, 2H), 2.23-2.11 (m, 2H), 2.00-1.82 (m, 4H), 1.73-1.57 (m, 3H), 1.50-1.24 (m, 18H), 0.90-0.81 (m, 9H) ppm. One proton of carboxyl group on TFA was revealed.

### 87-11b (Minority):

ESI m/z: 653.0 (M/3+H)⁺; 979.1 (M/2+H)⁺. ¹H NMR: (400 MHz, DMSO-d6) δ10.04 (s, 1H), 8.18 (d, J=7.6 Hz, 1H), 8.04 (d, J=8.8 Hz, 1H), 7.83-7.75 (m, 4H), 7.61 (d, J=8.8 Hz, 2H), 7.54 (d, J=8.8 Hz, 1H), 7.36 (d, J=8.8 Hz, 2H), 7.33 (s, 1H), 7.00 (d, J=8.4 Hz, 1H), 6.53 (s, 1H), 5.99 (s, 1H), 5.45-5.39 (m, 3H), 5.34-5.23 (m, 3H), 5.09 (s, 2H), 4.46 (t, J=4.2 Hz, 2H), 4.41-4.36 (m, 1H), 4.27-4.23 (m, 1H), 3.93-3.88 (m, 1H), 3.79 (t, J=4.6 Hz, 2H), 3.67-3.56 (m, 8H), 3.56-3.47 (m, 44H), 3.13-2.90 (m, 12H), 2.83 (t, J=7.6 Hz, 2H), 2.43 (t, J=7.6 Hz, 2H), 2.38 (s, 3H), 2.29 (t, J=6.4 Hz, 2H), 2.22-2.10 (m, 2H), 2.00-1.85 (m, 4H), 1.73-1.55 (m, 3H), 1.48-1.24 (m, 18H), 0.90-0.81 (m, 9H) ppm. One proton of carboxyl group on TFA was revealed.

### Step 7

Compound 87-11a (tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[4-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}pentyl]carbamate (87-11a, 60 mg, 0.031 mmol)) in methanol (2 mL) was stirred at room temperature, then 2M HCl in methanol (1 mL) was added. The solution was stirred for 1 h until LCMS showed that all starting material was consumed. Then the reaction solution was neutralized with aq. sodium bicarbonate solution, and solvent methanol was evaporated off with a rotary evaporator, and the residue was dissolved in water and purified by reverse phase flash chromatography (0.01% TFA) to collect the desired fractions, which were freeze-dried to yield product 87-12a ({4-[(2S)-2-[(2S)-2-[(2S)-2-amino-6-{1-[5-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (87-12a, 20 mg, 0.011 mmol)) as a white solid. ESI m/z: 928.7 (M/2+H)⁺, 619.5 (M/3+H)⁺. ¹H NMR: (400 MHz, DMSO-d6) δ 10.09 (s, 1H), 8.41 (d, J=8.4 Hz, 1H), 8.31 (d, J=8.0 Hz, 1H), 8.08-8.02 (m, 4H), 7.85-7.80 (m, 3H), 7.79 (s, 1H), 7.60 (d, J=8.8 Hz, 2H), 7.37 (d, J=8.4 Hz, 2H), 7.32 (s, 1H), 6.54 (s, 1H), 6.00 (t, J=4.0 Hz, 1H), 5.46 (brs, 4H), 5.34-5.25 (m, 3H), 5.09 (s, 2H), 4.47-4.26 (m, 7H), 3.89-3.84 (m, 1H), 3.80-3.76 (m, 2H), 3.60-3.56 (m, 5H), 3.50-3.47 (m, 44H), 3.29-3.22 (m, 2H), 3.16-2.94 (m, 9H), 2.85-2.81 (m, 1H), 2.45-2.41 (m, 1H), 2.39 (s, 3H), 2.31 (t, J=6.4 Hz, 2H), 2.24-2.11 (m, 4H), 2.02-1.98 (m, 1H), 1.90-1.82 (m, 3H), 1.69-1.64 (m, 3H), 1.60-1.55 (m, 1H), 1.46-1.23 (m, 7H), 0.92-0.86 (m, 9H) ppm. One proton of carboxyl group on TFA was revealed.

### Step 8

A solution of compound 87-12a ({4-[(2S)-2-[(2S)-2-[(2S)-2-amino-6-{1-[4-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (87-12a, 20 mg, 0.011 mmol)) and DIPEA (1.94 mg, 0.015 mmol) in anhydrous DMF (1 mL) was stirred at room temperature for 5 min, then a solution of compound 87-13 (2,5-dioxopyrrolidin-1-yl 6-(2,5-dioxo-2,5-dihydro-1H-pyrrol-1-yl)hexanoate (87-13, 3.08 mg, 0.010 mmol)) in anhydrous DMF (1 mL) was added dropwise by syringe. The resulting solution was stirred for another 4 h until LCMS indicated all starting activated ester was consumed and little starting amine was remained. The resulting solution was then acidified to pH 4-5 with TFA, purified by Prep-HPLC (0.01% TFA) to yield desired fractions, which was freeze-dried to yield PB087 ({4-[(2S)-5-(carbamoylamino)-2-[(2S)-2-[(2S)-2-[6-(2,5-dioxo-2,5-dihydro-1H-pyrrol-1-yl)hexanamido]-6-{1-[4-(2-{[(2S,3R,4R,5R)-2,3,4,5,6-pentahydroxyhexyl]carbamoyl}ethyl)-1H-1,2,3-triazol-1-yl]-3,6,9,12,15,18,21,24,27,30,33,36-dodecaoxanonatriacontan-39-amido}hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (PB087, 12 mg, 0.006 mmol, 58.56%)) as a white solid. ESI m/z: 1047.7 (M/2+Na)⁺, 684.1 (M/3+H)⁺, 513.4 (M/4+H)⁺, retention time 6.372 min (HPLC).

¹H NMR: (400 MHz, DMSO-d6) δ 10.04 (s, 1H), 8.13 (d, J=6.8 Hz, 1H), 8.09-8.05 (m, 1H), 7.97 (d, J=8.0 Hz, 1H), 7.86-7.76 (m, 4H), 7.69-7.66 (m, 1H), 7.60 (d, J=8.4 Hz, 2H), 7.36 (d, J=8.4 Hz, 2H), 7.32 (s, 1H), 6.99 (s, 2H), 6.55 (s, 1H), 6.05-5.95 (m, 1H), 5.45 (s, 4H), 5.33-5.24 (m, 3H), 5.08 (s, 2H), 4.47-4.33 (m, 4H), 4.27-4.16 (m, 3H), 3.80-3.75 (m, 2H), 3.67-3.55 (m, 12H), 3.50-3.48 (m, 44H), 3.37-3.34 (m, 2H), 3.29-3.22 (m, 2H), 3.13-2.90 (m, 8H), 2.83 (t, J=7.6 Hz, 1H), 2.43 (d, J=7.60 Hz, 1H), 2.38 (s, 3H), 2.29 (t, J=6.4 Hz, 2H), 2.22-2.05 (m, 4H), 2.00-1.83 (m, 3H), 1.73-1.57 (m, 3H), 1.51-1.32 (m, 9H), 1.24-1.14 (m, 4H), 0.90-0.81 (m, 9H) ppm. One proton of carboxyl group on TFA was revealed. Drug linker PB087 can be used to make a conjugate such as PA087.

### Example 19: Preparation of the Drug-Linker (PB088) Containing a PEG Unit and a Cleavable Linker Attached to Exatecan

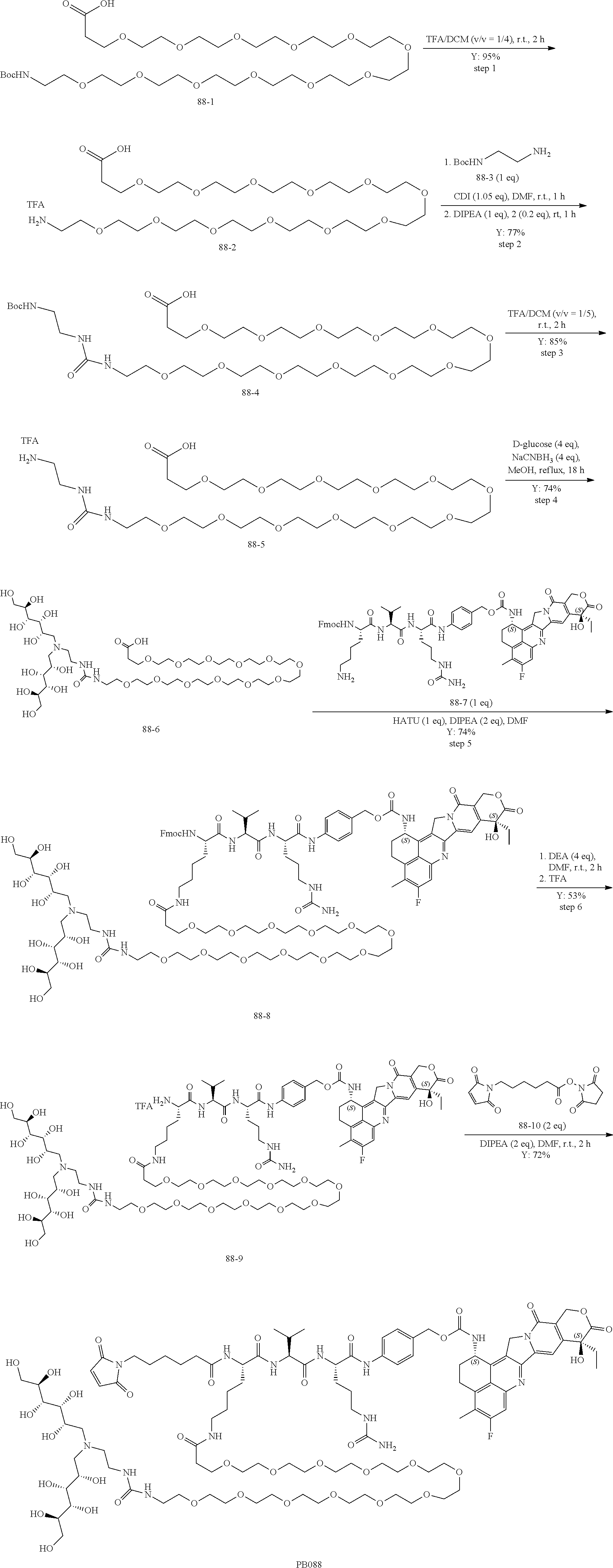

A Drug-Linker containing a PEG unit and a cleavable linker attached to exatecan (PB088) was prepared as follows:

### Step 1

To a solution of compound 88-1 (1-{[(tert-butoxy)carbonyl]amino}-3,6,9,12,15,18,21,24,27,30,33,36-dodecaoxanonatriacontan-39-oic acid (88-1, 500 mg, 0.697 mmol) in DCM (4 mL) was added TFA (1 mL, 13.463 mmol). The mixture was stirred at room temperature for 2 hours. Then the solution was concentrated and the crude mixture was purified by reverse phase flash chromatography (0.01% TFA) to yield compound 88-2 (1-amino-3,6,9,12,15,18,21,24,27,30,33,36-dodecaoxanonatriacontan-39-oic acid (88-2, 420 mg, 0.680 mmol, 97.59%)) as colorless oil. ESI m/z: 618.4 (M+H)⁺.

### Step 2

A solution of compound 88-3 (tert-butyl N-(2-aminoethyl)carbamate (88-3, 0.335 mL, 2.122 mmol)) and CDI (0.264 mL, 2.121 mmol) in DMF (3 mL) was stirred at room temperature for 1 h to prepare activated intermediate, then compound 88-2 (1-amino-3,6,9,12,15,18,21,24,27,30,33,36-dodecaoxanonatriacontan-39-oic acid (88-2, 236 mg, 0.382 mmol)) and DIPEA (197.48 mg, 1.528 mmol) were added. The resulting solution was stirred for another 1 h until all starting materials were consumed. The reaction mixture was purified by reverse phase flash chromatography (0.01% TFA) to yield the desired fractions, which were freeze-dried to yield compound 88-4 (1-{[(2-{[(tert-butoxy)carbonyl]amino}ethyl)carbamoyl]amino}-3,6,9,12,15,18,21,24,27,30,33,36-dodecaoxanonatriacontan-39-oic acid (88-4, 230 mg, 0.286 mmol, 74.89%)) as a colorless oil. ESI m/z: =804.5 (M+H)⁺. ¹H NMR (400 MHz, DMSO) δ 12.17 (s, 1H), 6.78 (t, J=4.8 Hz, 1H), 6.08-5.81 (m, 2H), 3.60 (t, J=6.4 Hz, 2H), 3.54-3.48 (m, 44H), 3.37 (t, J=5.6 Hz, 3H), 3.17-3.12 (m, 2H), 3.05-2.96 (m, 2H), 2.93-2.87 (m, 2H), 2.44 (t, J=6.4 Hz, 2H), 1.37 (s, 9H) ppm.

### Step 3

To a solution of compound 88-4 (1-{[(2-{[(tert-butoxy)carbonyl]amino}ethyl)carbamoyl]amino}-3,6,9,12,15,18,21,24,27,30,33,36-dodecaoxanonatriacontan-39-oic acid (88-4, 430 mg, 0.535 mmol)) in DCM (5 mL) was added TFA (1 mL, 13.463 mmol). The mixture was stirred at room temperature for 2 hours. Then the solution was concentrated and the crude material was purified by reverse phase flash chromatography (0.01% TFA) to yield a TFA salt of compound 88-5 (1-{[(2-aminoethyl)carbamoyl]amino}-3,6,9,12,15,18,21,24,27,30,33,36-dodecaoxanonatriacontan-39-oic acid (88-5, 320 mg, 0.455 mmol, 85.00%)) as a colorless oil. ESI m/z: =704.5 (M+H)⁺.

### Step 4

To a solution of compound 88-5 (1-{[(2-aminoethyl)carbamoyl]amino}-3,6,9,12,15,18,21,24,27,30,33,36-dodecaoxanonatriacontan-39-oic acid (88-5, 163 mg, 0.232 mmol)) in MeOH (20 mL) was added D-glucose (166.90 mg, 0.926 mmol) in portions, and the mixture was heated to 85° C. with stirring for 30 minutes under a N₂ atmosphere. Then NaCNBH₃ (58.21 mg, 0.926 mmol) was added. The reaction mixture was stirred with heating for 18 h to complete the reaction. Then the reaction solution was concentrated to dryness and purified by reverse phase flash chromatography (0.01% TFA) to yield the desired fractions, which were freeze-dried to yield compound 88-6 (1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-oic acid (88-6, 200 mg, 0.194 mmol, 83.52%)) as a colorless oil. ESI m/z: 1032.5 (M+H)⁺. ¹H NMR (400 MHz, DMSO) δ 6.40-6.38 (m, 2H), 4.67 (brs, 8H), 3.70-3.63 (m, 2H), 3.61-3.39 (m, 54H), 3.38-3.37 (m, 6H), 3.22-2.91 (m, 6H), 2.49-2.35 (m, 4H), 2.31 (t, J=6.4 Hz, 2H) ppm. Proton of carboxyl group was not revealed.

### Step 5

A mixture of compound 88-6 (1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-oic acid (88-6, 138.45 mg, 0.134 mmol)), HATU (50.95 mg, 0.134 mmol) and DIPEA (34.61 mg, 0.268 mmol) in DMF (3 mL) was stirred for 15 minutes at room temperature under N₂. Then compound 88-7 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (88-7, 160 mg, 0.134 mmol)) was added slowly, and the reaction mixture was stirred for 2 h. After completion of the reaction (monitored by LCMS), the reaction solution was purified by prep-HPLC (0.01% TFA) to afford compound 88-8 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-({[(9H-fluoren-9-yl)methoxy]carbonyl}amino)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (88-8, 220 mg, 0.100 mmol, 74.27%)) as a pale yellow solid. ESI m/z: 1103.6 (M/2+H)⁺.

### Step 6

To a solution of compound 88-8 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-({[(9H-fluoren-9-yl)methoxy]carbonyl}amino)hexanamido]-3-methylbutanamido]-5-(carbamoylamino)pentanamido]phenyl}methyl N—[(I10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (88-8, 230 mg, 0.104 mmol)) in DMF (2 mL) was added diethyl amine (30.51 mg, 0.417 mmol). The mixture was stirred at room temperature for 2 hours, then the solution was concentrated under vacuo to remove most of diethyl amine, and the crude material was purified by reverse phase flash chromatography (0.01% TFA) to yield a TFA salt of compound 88-9 ({4-[(2S)-2-[(2S)-2-[(2S)-2-amino-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (88-9, 150 mg, 0.076 mmol, 72.53%)) as a pale yellow solid. ESI m/z: 992.6 (M/2+H)⁺.

### Step 7

A solution of compound 88-9 ({4-[(2S)-2-[(2S)-2-[(2S)-2-amino-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}.0\hat{ }{20,24}]tetracosa-1,6(11),12,14,16,18,20(24)-heptaen-23-yl]carbamate (88-9, TFA salt, 52 mg, 0.026 mmol)) in DMF (2 mL) was added compound 88-10 (2,5-dioxopyrrolidin-1-yl 6-(2,5-dioxo-2,5-dihydro-1H-pyrrol-1-yl)hexanoate (88-10, 16.17 mg, 0.052 mmol)) and DIPEA (6.78 mg, 0.052 mmol) at room temperature. After addition, the solution was stirred for 2 h. until LCMS showed that the reaction was completed. Then the reaction solution was purified by Prep-HPLC (0.01% TFA) to yield the desired fractions, which were freeze-dried to yield PB088 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB088, 40 mg, 0.018 mmol, 70.09%)) as a pale yellow solid. ESI m/z: 1089.6 (M/2+H)⁺, 726.3 (M/3+H)⁺, retention time 6.007 min (HPLC). ¹H NMR (400 MHz, DMSO) δ 10.02 (s, 1H), 8.67 (s, 1H), 8.11 (d, J=7.2 Hz, 1H), 8.06 (d, J=8.4 Hz, 1H), 7.95 (d, J=8.0 Hz, 1H), 7.79 (dd, J=11.0, 5.7 Hz, 2H), 7.66 (d, J=8.9 Hz, 1H), 7.60 (d, J=8.4 Hz, 2H), 7.36 (d, J=8.4 Hz, 2H), 7.31 (s, 1H), 7.00 (s, 2H), 6.53 (s, 1H), 6.-6.23 (m, 2H), 5.99 (dd, J=9.8, 4.1 Hz, 1H), 5.43 (d, J=12.1 Hz, 4H), 5.35 (s, 2H), 5.29 (s, 3H), 5.07 (s, 2H), 4.75-4.37 (m, 8H), 4.25-4.17 (m, 2H), 4.00 (d, J=7.6 Hz, 2H), 3.69-3.66 (m, 2H), 3.59-3.54 (m, 5H), 3.52-3.45 (m, 54H), 3.23-3.11 (m, 5H), 3.09-2.85 (m, 5H), 2.38 (s, 3H), 2.35-1.79 (m, 11H), 1.72-1.55 (m, 3H), 1.51-1.42 (m, 6H), 1.42-1.10 (m, 9H), 0.89-0.80 (m, 9H) ppm.

Drug linker PB088 can be used to make a conjugate such as PA088.

### Example 20: Preparation of the Drug-Linker (PB089) Containing a PEG Unit and a Cleavable Linker Attached to Exatecan

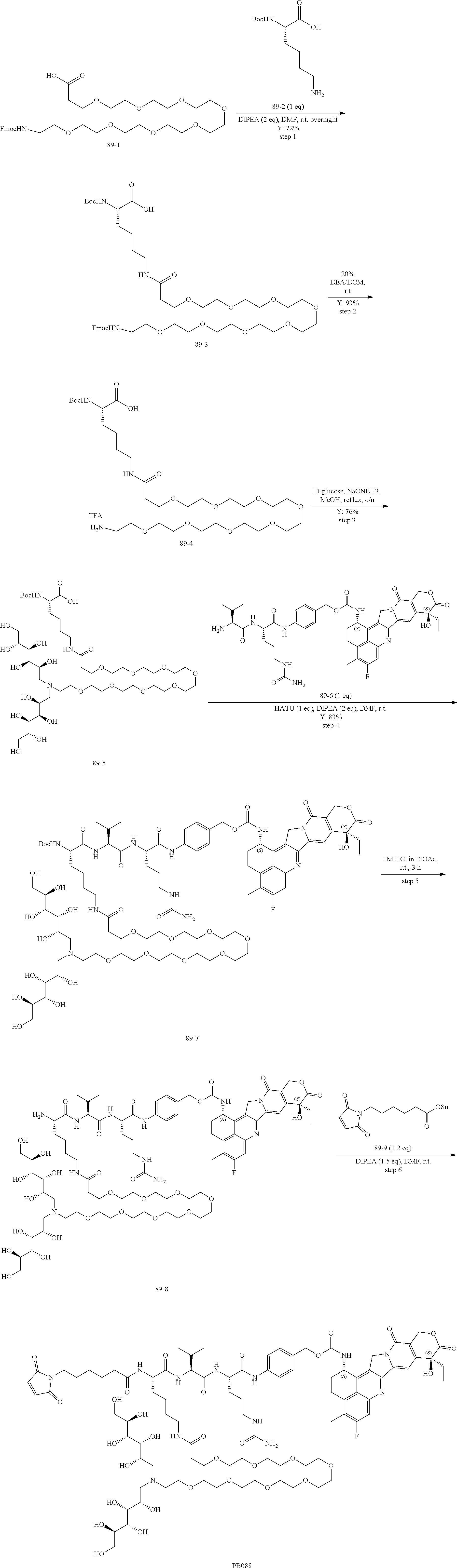

A Drug-Linker containing a PEG unit and a cleavable linker attached to exatecan (PB089) was prepared as follows:

### Step 1

A solution of compound 89-1 (2,5-dioxopyrrolidin-1-yl 1-({[(9H-fluoren-9-yl)methoxy]carbonyl}amino)-3,6,9,12,15,18,21,24-octaoxaheptacosan-27-oate (89-1, 1.83 g, 2.405 mmol)), compound 89-2 ((2S)-6-amino-2-{[(tert-butoxy)carbonyl]amino}hexanoic acid (89-2, 0.59 g, 2.405 mmol)) and DIPEA (0.62 g, 4.811 mmol) in DMF (10 mL) was stirred at room temperature for 18 h until LCMS indicated complete conversion. Then the reaction solution was purified by reverse phase flash chromatography (0.01% TFA) to yield the desired fractions, which were freeze-dried to yield compound 89-3 ((2S)-2-{[(tert-butoxy)carbonyl]amino}-6-[1-({[(9H-fluoren-9-yl)methoxy]carbonyl}amino)-3,6,9,12,15,18,21,24-octaoxaheptacosan-27-amido]hexanoic acid (89-3, 1.54 g, 1.726 mmol, 71.77%)) as a white solid. ESI m/z: 914.5 (M+Na)⁺, 396.8 (M−100)/2+H)⁺.

### Step 2

To a solution of compound 89-3 ((2S)-2-{[(tert-butoxy)carbonyl]amino}-6-[1-({[(9H-fluoren-9-yl)methoxy]carbonyl}amino)-3,6,9,12,15,18,21,24-octaoxaheptacosan-27-amido]hexanoic acid (89-3, 1.14 g, 1.278 mmol)) in DCM (10 mL) was added diethyl amine (0.401 mL, 5.112 mmol). The reaction mixture was stirred at room temperature for 2 hours to completion. Then the mixture was purified by reverse phase flash chromatography to yield compound 89-4 ((2S)-6-(1-amino-3,6,9,12,15,18,21,24-octaoxaheptacosan-27-amido)-2-{[(tert-butoxy)carbonyl]amino}hexanoic acid (89-4, 800 mg, 1.194 mmol, 93.46%)) as a colorless oil. ESI m/z=670.5 (M+H)⁺, 285.8 ((M−100)/2+H)⁺.

### Step 3

To a solution of compound 89-4 ((2S)-6-(1-amino-3,6,9,12,15,18,21,24-octaoxaheptacosan-27-amido)-2-{[(tert-butoxy)carbonyl]amino}hexanoic acid (89-4, 800 mg, 1.194 mmol)) in MeOH (50 mL) was added D-glucose (860.80 mg, 4.778 mmol) in portions, and the mixture was heated to 85° C. with stirring for 30 minutes under a N₂ atmosphere. Then NaCNBH₃ (300.12 mg, 4.776 mmol) was added. After addition, the reaction mixture was heated under reflux for 18 h. Then the reaction solution was concentrated and purified by reverse phase flash chromatography (0.01% TFA) to yield the desired fractions, which were freeze-dried to yield compound 89-5 ((2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanoic acid (89-5, 966 mg, 0.968 mmol, 81.06%)) as a colorless oil. ESI m/z: 998.5 (M+H)⁺, 449.8 (M/2+H)⁺.

### Step 4

A mixture of compound 89-5 ((2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanoic acid (89-5, 275 mg, 0.276 mmol)), HATU (91.67 mg, 0.241 mmol) and DIPEA (59.33 mg, 0.459 mmol) in DMF (5 mL) was stirred for 15 minutes at room temperature. Then compound 89-6 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (89-6, 193.07 mg, 0.230 mmol)) was added slowly, and the reaction mixture was stirred for 2 h. The reaction solution was purified by Prep-HPLC (0.01% TFA) to afford compound 89-7 ({4-[(2S)-2-[(2S)-2-[(2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (89-7, 347 mg, 0.191 mmol, 82.99%)) as a pale yellow solid. ESI m/z: 911.0 (M/2+H)⁺, 607.9 (M/3+H)⁺.

### Step 5

A solution of compound 89-7 ({4-[(2S)-2-[(2S)-2-[(2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (89-7, 347 mg, 0.191 mmol)) in 1M HCl in ethyl acetate (219 mg, 6.007 mmol) was stirred at room temperature for 2 h until LCMS showed that the reaction was completed. Then solvent was evaporated with a rotary evaporator and the residue was purified by reverse phase flash chromatography (0.01% TFA) to afford compound 89-8 ({4-[(2S)-2-[(2S)-2-[(2S)-2-amino-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (89-8, 93 mg, 0.054 mmol, 28.36%)) as a white solid. ESI=574.5 (M/3+H)⁺, 861.2 (M/2+H)⁺.

### Step 6

To a solution of compound 89-8 ({4-[(2S)-2-[(2S)-2-[(2S)-2-amino-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (89-8, 93 mg, 0.054 mmol)) in DMF (2 mL) was added DIPEA (10.48 mg, 0.081 mmol) and compound 89-9 (2,5-dioxopyrrolidin-1-yl 6-(2,5-dioxo-2,5-dihydro-1H-pyrrol-1-yl)hexanoate (89-9, 24.99 mg, 0.081 mmol)). After addition, the solution was stirred at room temperature for 1 h until all starting amine was consumed (monitored by LMCS). Then the resulting solution was adjusted to pH 6 and purified by Prep-HPLC (0.01% TFA) to afford a TFA salt of product PB089 ({4-[(2S)-5-(carbamoylamino)-2-[(2S)-2-[(2S)-2-[6-(2,5-dioxo-2,5-dihydro-1H-pyrrol-1-yl)hexanamido]-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (PB089, 55 mg, 0.029 mmol, 53.17%)) as a white solid. ESI m/z: 639.1 (M/3+H)⁺, retention time 5.799 min (HPLC). ¹H NMR (400 MHz, DMSO-d6) δ 10.04 (s, 1H), 8.14-8.12 (m, 2H), 8.07 (d, J=8.4 Hz, 1H), 7.97 (d, J=7.6 Hz, 1H), 7.83 (t, J=5.6 Hz, 1H), 7.76 (d, J=11.2 Hz, 1H), 7.67 (d, J=8.8 Hz, 1H), 7.60 (d, J=8.4 Hz, 2H), 7.36 (d, J=8.4 Hz, 2H), 7.31 (s, 1H), 6.99 (s, 2H), 6.548 (s, 1H), 6.02 (t, J=5.6 Hz, 1H), 5.45 (brs, 6H), 5.33-5.21 (m, 3H), 5.08 (s, 2H), 4.82 (brs, 2H), 4.56 (brs, 4H), 4.40-4.17 (m, 5H), 3.99 (brs, 2H), 3.80-3.76 (m, 2H), 3.69-3.67 (m, 2H), 3.62-3.56 (m, 8H), 3.49-3.45 (m, 28H), 3.36-2.90 (m, 16H), 2.37 (s, 3H), 2.29 (t, J=6.4 Hz, 2H), 2.24-2.08 (m, 4H), 2.00-1.81 (m, 3H), 1.69-1.57 (m, 3H), 1.52-1.42 (m, 6H), 1.36-1.29 (m, 3H), 1.22-1.14 (m, 4H), 0.89-0.81 (m, 9H) ppm.

Drug linker PB089 can be used to make a conjugate such as PA089.

### Example 21: Preparation of the Drug-Linker (PB090) Containing a PEG Unit and a Cleavable Linker Attached to Exatecan

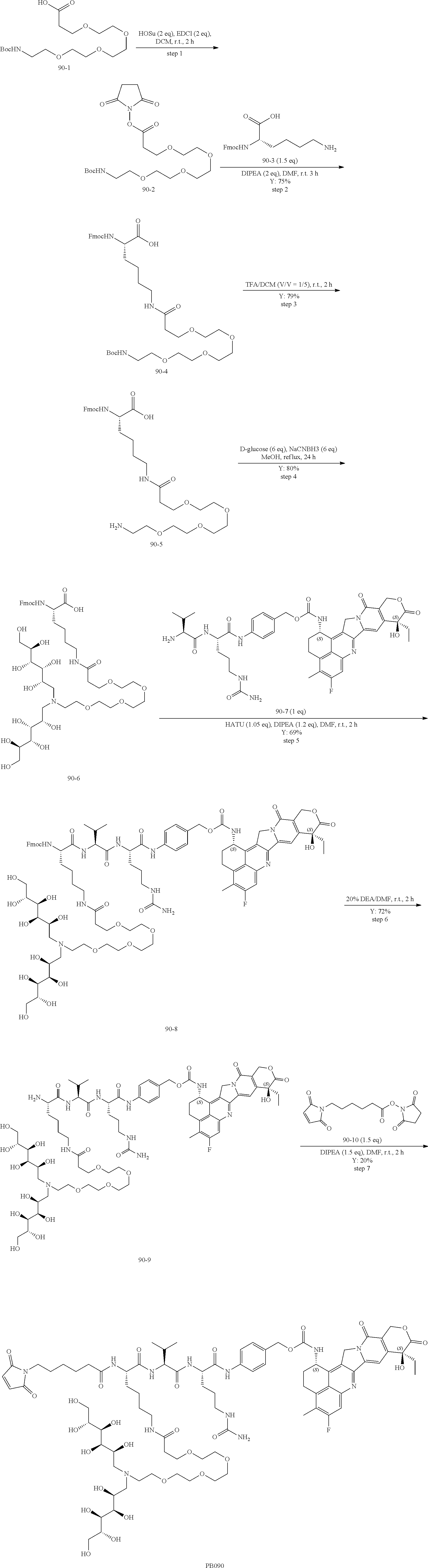

A Drug-Linker containing a PEG unit and a cleavable linker attached to exatecan (PB090) was prepared as follows:

### Step 1

To a solution of compound 90-1 (1-{[(tert-butoxy)carbonyl]amino}-3,6,9,12-tetraoxapentadecan-15-oic acid (90-1, 3.737 mL, 11.494 mmol)) in DCM (15 mL) was added HOSu (1.98 g, 17.240 mmol) and EDCI (3.30 g, 17.240 mmol). The mixture was stirred at room temperature for 2 h. Then the resulting solution was washed with brine (20 mL) and extracted with DCM (20 mL). The collected organic layer was dried with Na₂SO₄, filtered and the filtrate was evaporated to dryness, affording product 90-2 (2,5-dioxopyrrolidin-1-yl 2,2-dimethyl-4-oxo-3,8,11,14,17-pentaoxa-5-azaicosan-20-oate (90-2, 5.32 g, 11.503 mmol, 100%)). ESI m/z: 485.3 (M+H)⁺.

### Step 2

To a solution of compound 90-2 (2,5-dioxopyrrolidin-1-yl 2,2-dimethyl-4-oxo-3,8,11,14,17-pentaoxa-5-azaicosan-20-oate (90-2, 5.32 g, 11.503 mmol)) in DMF (20 mL) was added compound 90-3 ((2S)-6-amino-2-({[(9H-fluoren-9-yl) methoxy]carbonyl}amino) hexanoic acid (90-3, 6.36 g, 17.254 mmol)) and DIPEA (2.97 g, 23.005 mmol). The mixture was stirred at room temperature for 3 h. The resulting solution was purified by reverse phase separation (0.01% TFA) to afford the product 90-4 ((2S)-6-(1-{[(tert-butoxy) carbonyl]amino}-3,6,9,12-tetraoxapentadecan-15-amido)-2-({[(9H-fluoren-9-yl) methoxy]carbonyl}amino) hexanoic acid (90-4, 6.18 g, 8.637 mmol, 75.12%)) as a colorless oil. ESI m/z: 716.5 (M+H)⁺.

### Step 3

To a solution of compound 90-4 ((2S)-6-(1-{[(tert-butoxy)carbonyl]amino}-3,6,9,12-tetraoxapentadecan-15-amido)-2-({[(9H-fluoren-9-yl)methoxy]carbonyl}amino)hexanoic acid (90-4, 6.18 g, 8.633 mmol)) in DCM (20 mL) was added TFA (4 mL, 53.850 mmol). The mixture was stirred at room temperature for 2 h. The resulting solution was evaporated and purified by reverse phase flash chromatography (0.01% TFA) to afford the product 90-5 ((2S)-6-(1-amino-3,6,9,12-tetraoxapentadecan-15-amido)-2-({[(9H-fluoren-9-yl)methoxy]carbonyl}amino)hexanoic acid (90-5, 4.2 g, 6.821 mmol, 79.01%)) as colorless oil. ESI m/z: 616.4 (M+H)⁺.

### Step 4

To a solution of compound 90-5 ((2S)-6-(1-amino-3,6,9,12-tetraoxapentadecan-15-amido)-2-({[(9H-fluoren-9-yl)methoxy]carbonyl}amino)hexanoic acid (90-5, 4.2 g, 6.821 mmol)) in MeOH (25 mL) was added D-glucose (7.37 g, 40.926 mmol) and NaBH₃CN (2.57 g, 40.926 mmol). The mixture was stirred at 60° C. for 24 h. The resulting solution was concentrated and purified by reverse phase flash chromatography (0.01% TFA) to compound 90-6 (afford the product (2S)-2-({[(9H-fluoren-9-yl)methoxy]carbonyl}amino)-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanoic acid (90-6, 5.16 g, 5.466 mmol, 80.12%)) as a colorless oil. ESI m/z: 945.6 (M+H)⁺.

### Step 5

To a solution of compound 90-6 ((2S)-2-({[(9H-fluoren-9-yl)methoxy]carbonyl}amino)-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanoic acid (90-6, 168 mg, 0.178 mmol)) in DMF (5 mL) was added compound 90-7 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (90-7, 150 mg, 0.178 mmol)), DIPEA (27.58 mg, 0.214 mmol) and HATU (71.05 mg, 0.187 mmol). The mixture was stirred at room temperature for 1.5 h. The resulting solution was adjusted to pH 6 and purified by reverse phase flash chromatography (0.01% TFA) to afford the product 90-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]pentyl]carbamate (90-8, 217 mg, 0.123 mmol, 69.01%)). ESI m/z: 884.1 (M/2+H)⁺.

### Step 6

To a solution of compound 90-8 ((9H-fluoren-9-yl)methyl N-[(1S)-1-{[(1S)-1-{[(1S)-4-(carbamoylamino)-1-({4-[({[(10S,23S)-10-ethyl-8-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]pentyl]carbamate (90-8, 217 mg, 0.123 mmol)) in DMF (2 mL) was added diethyl amine (0.4 mL, 2.501 mmol). The mixture was stirred at room temperature for 2 h. The resulting solution was adjusted to pH 6 and purified by reverse phase flash chromatography (0.01% TFA) to afford the product 90-9 ({4-[(2S)-2-[(2S)-2-[(2S)-2-amino-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (90-9, 138 mg, 0.089 mmol, 72.74%)) as a white solid. ESI m/z: 773.5 (M/2+H)⁺.

### Step 7

To a solution of compound 90-9 ({4-[(2S)-2-[(2S)-2-[(2S)-2-amino-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (90-9, 138 mg, 0.089 mmol)) in DMF (2 mL) was added DIPEA (17.32 mg, 0.134 mmol) and compound 90-10 (2,5-dioxopyrrolidin-1-yl 6-(2,5-dioxo-2,5-dihydro-1H-pyrrol-1-yl)hexanoate (90-10, 41.31 mg, 0.134 mmol)). The mixture was stirred at room temperature for 2 h. The resulting solution was adjusted to pH 6 and purified by Prep-HPLC (0.01% TFA) to afford TFA salt of product PB090 ({4-[(2S)-5-(carbamoylamino)-2-[(2S)-2-[(2S)-2-[6-(2,5-dioxo-2,5-dihydro-1H-pyrrol-1-yl)hexanamido]-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]pentanamido]phenyl}methyl N-[(10S,23S)-10-ethyl-8-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (PB090, 32 mg, 0.018 mmol, 20.61%)) as a white solid. ESI m/z: 580.5 (M/3+H)⁺, retention time 6.558 min (HPLC). ¹H NMR (400 MHz, DMSO-d6) δ 10.03 (s, 1H), 8.14-8.12 (d, J=6.8 Hz, 2H), 8.08-8.05 (d, J=8.8 Hz, 1H), 7.98-7.96 (d, J=8.0 Hz, 1H), 7.83-7.81 (t, J=5.6 Hz, 1H), 7.78-7.76 (d, J=10.8 Hz, 1H), 7.67-7.64 (d, J=8.0 Hz, 1H), 7.61-7.59 (d, J=8.0 Hz, 2H), 7.37-7.35 (d, J=8.4 Hz, 2H), 7.31 (s, 1H), 7.00 (s, 2H), 6.54 (s, 1H), 6.01 (t, J=5.6 Hz, 1H), 5.45 (brs, 6H), 5.29-5.27 (m, 3H), 5.08 (s, 2H), 4.86-4.74 (m, 2H), 4.65-4.40 (m, 5H), 4.40-4.35 (m, 1H), 4.27-4.17 (m, 2H), 4.04-3.94 (m, 2H), 3.79-3.76 (m, 2H), 3.69-3.67 (m, 2H), 3.67-3.54 (m, 8H), 3.53-3.42 (m, 18H), 3.29-3.27 (m, 4H), 3.20-2.90 (m, 6H), 2.37 (s, 3H), 2.29 (t, J=6.4 Hz, 2H), 2.22-2.08 (m, 4H), 2.02-1.81 (m, 3H), 1.71-1.56 (m, 3H), 1.52-1.42 (m, 6H), 1.40-1.14 (m, 8H), 0.89-0.81 (m, 9H) ppm. One proton of carboxyl group on TFA was revealed.

Drug linker PB090 can be used to make a conjugate such as PA090.

### Example 22: Preparation of the Drug-Linker (PB091) Containing a PEG Unit and a Cleavable Linker Attached to Exatecan

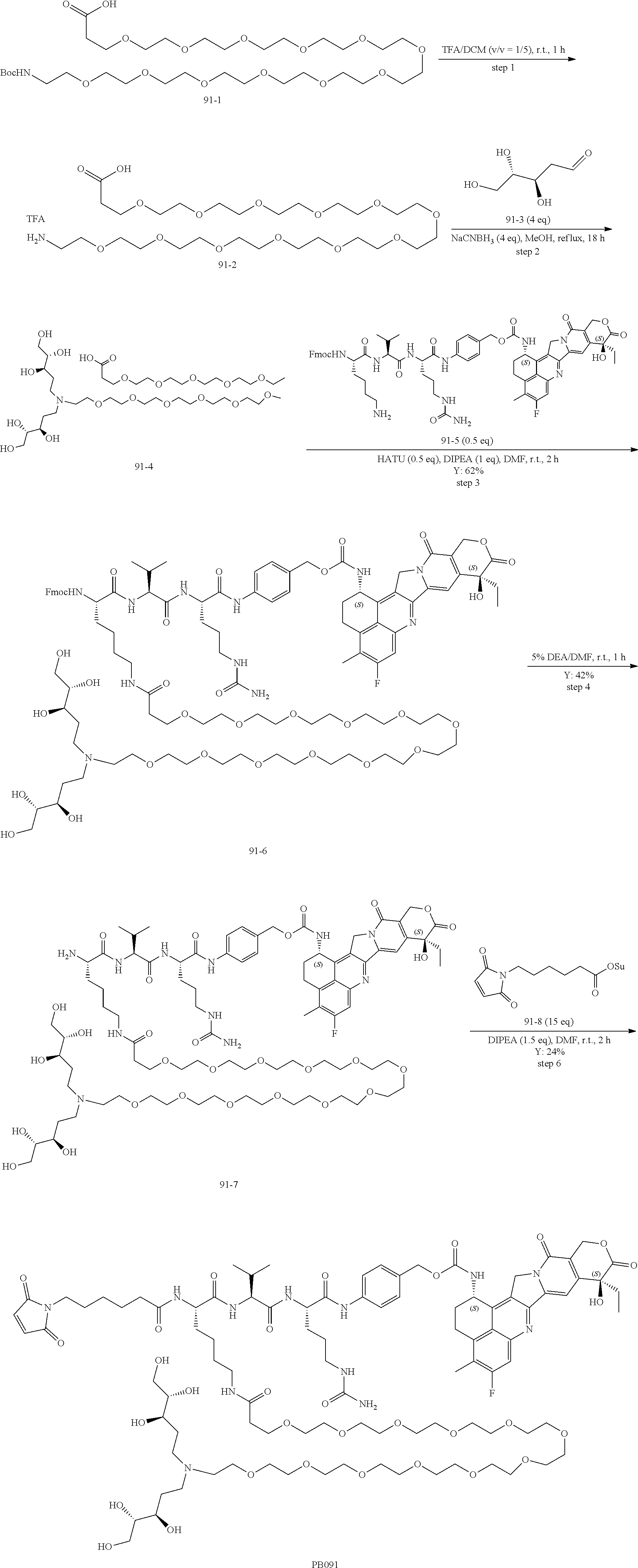

A Drug-Linker containing a PEG unit and a cleavable linker attached to exatecan (PB091) was prepared as follows:

### Step 1

A solution of compound 91-1 (1-{[(tert-butoxy)carbonyl]amino}-3,6,9,12,15,18,21,24,27,30,33,36-dodecaoxanonatriacontan-39-oic acid (91-1, 560 mg, 0.781 mmol)) in DCM (10 mL) was treated with TFA (2 mL, 26.925 mmol) and stirred at room temperature for 1 h until LCMS showed that the reaction was completed. Then the solution was concentrated, and the residue was suspended in DCM and concentrated again to yield crude product 91-2 (1-amino-3,6,9,12,15,18,21,24,27,30,33,36-dodecaoxanonatriacontan-39-oic acid, TFA salt (91-2, 600 mg, 0.821 mmol, 105.09%)) as a white solid. The product was used as such in the next step. ESI m/z=618.5 (M+H)⁺.

### Step 2

A reaction solution of compound 91-2 (1-amino-3,6,9,12,15,18,21,24,27,30,33,36-dodecaoxanonatriacontan-39-oic acid, TFA salt (91-2, 570.91 mg, 0.781 mmol)), compound 91-3 ((3R,4S)-3,4,5-trihydroxypentanal (91-3, 0.395 mL, 3.124 mmol)) and NaCNBH₃ (0.121 mL, 3.124 mmol) in methanol (16 mL) was heated under reflux under N₂ for 18 h until the reaction was complete as indicated by LCMS. The solvents were evaporated, and the residue was dissolved in water, and then purified by reverse phase flash chromatography (0.01% TFA) to yield the desired product 91-4 ((43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanoic acid (91-4, 700 mg, 0.821 mmol, 105.08%)) as a white foam, mixed with excess ribose. ESI m/z: 427.8 (M/2+H)⁺, 854.6 (M+H)⁺.

### Step 3

A solution of compound 91-4 ((43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanoic acid (91-4, 214.68 mg, 0.252 mmol)), compound 91-5 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (91-5, 150 mg, 0.126 mmol)) and DIPEA (32.47 mg, 0.252 mmol) in anhydrous DMF (2.0 mL) was stirred at room temperature for 5 min, and then a solution of HATU (47.85 mg, 0.126 mmol) in anhydrous DMF (0.5 mL) was added slowly. After addition, the resulting solution was stirred for another 2 h at room temperature until LCMS indicated complete reaction. The reaction solution was purified directly by reverse phase flash chromatography (0.01% TFA) to yield compound 91-6 ((9H-fluoren-9-yl)methyl N-[(1S)-1-{[(1S)-1-{[(1S)-4-(carbamoylamino)-1-({4-[(I{[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]pentyl]carbamate (91-6, 160 mg, 0.079 mmol, 62.72%)) as a white solid. ESI m/z: 507.8 (M/4+H)⁺, 676.7 (M/3+H)⁺.

### Step 4

A solution of compound 91-6 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]pentyl]carbamate (91-6, 80 mg, 0.039 mmol)) in DMF (1.5 mL) was stirred at room temperature and diethyl amine (0.08 mL, 0.777 mmol) was added. The resulting solution was stirred for another 1 h until LCMS showed that the reaction was completed. Diethyl amine was removed under vacuo, and the residue in DMF was purified by Prep-HPLC (10 mM ammonia bicarbonate) to yield the expected product 91-7 ({4-[(2S)-2-[(2S)-2-[(2S)-2-amino-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (91-7, 30 mg, 0.017 mmol, 42.12%)) as a white solid. ESI m/z: 903.7 (M/2+H)⁺, 602.7 (M/3+H)⁺. ¹H NMR: (400 MHz, DMSO-d6) δ 10.11 (s, 1H), 8.35-8.31 (m, 5H), 8.09-8.06 (m, 2H), 7.83-7.77 (m, 2H), 7.61 (d, J=8.8 Hz, 2H), 7.36 (d, J=8.4 Hz, 2H), 7.32 (s, 1H), 6.09 (s, 1H), 5.46 (s, 4H), 5.30-5.29 (m, 3H), 5.08 (s, 2H), 4.41-4.34 (m, 1H), 4.27-4.23 (m, 1H), 3.59-3.56 (m, 4H), 3.50-3.47 (m, 44H), 3.47-3.46 (m, 2H), 3.36-3.30 (m, 4H), 3.30-3.22 (m, 4H), 3.14-3.08 (m, 2H), 3.03-2.95 (m, 4H), 2.68-2.56 (m, 6H), 2.38 (s, 3H), 2.29 (t, J=6.4 Hz, 2H), 2.22-2.13 (m, 4H), 2.00-1.84 (m, 4H), 1.76-1.65 (m, 4H), 1.65-1.56 (m, 3H), 1.46-1.28 (m, 10H), 0.90-0.81 (m, 9H) ppm. Two protons of carboxyl group on TFA was revealed between 8.35-8.31 ppm.

### Step 5

To a solution of compound 91-7 ({4-[(2S)-2-[(2S)-2-[(2S)-2-amino-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (91-7, 30 mg, 0.017 mmol)) in DMF (2 mL) was added DIPEA (3.30 mg, 0.026 mmol) and compound 91-8 (2,5-dioxopyrrolidin-1-yl 6-(2,5-dioxo-2,5-dihydro-1H-pyrrol-1-yl)hexanoate (91-8, 7.86 mg, 0.026 mmol)). The mixture was stirred at room temperature for 2 h. The resulting solution was purified by Prep-HPLC (0.01% TFA) to afford the product PB091 ({4-[(2S)-5-(carbamoylamino)-2-[(2S)-2-[(2S)-2-[6-(2,5-dioxo-2,5-dihydro-1H-pyrrol-1-yl)hexanamido]-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (PB091, 7.5 mg, 0.004 mmol, 22.58%)) as a white solid. ESI m/z: 667.0 (M/3+H)⁺, 500.8 (M/4+H)⁺, retention time 5.791 min (HPLC). ¹H NMR (400 MHz, DMSO-d6) δ 10.03 (s, 1H), 9.10 (brs, 1H), 8.13-8.10 (d, J=7.2 Hz, 1H), 8.07-8.05 (d, J=8.8 Hz, 1H), 7.98-7.96 (d, J=8.0 Hz, 1H), 7.83-7.80 (t, J=5.2 Hz, 1H), 7.77-7.74 (d, J=10.8 Hz, 1H), 7.68-7.65 (d, J=8.4 Hz, 1H), 7.61-7.59 (d, J=8.0 Hz, 2H), 7.38-7.35 (d, J=8.4 Hz, 2H), 7.31 (s, 1H), 6.99 (s, 2H), 6.54 (s, 1H), 6.02-6.01 (m, 1H), 5.45 (brs, 4H), 5.29-5.26 (m, 3H), 5.08 (s, 2H), 4.91 (brs, 2H), 4.76 (brs, 2H), 4.52 (brs, 2H), 4.38-4.36 (m, 1H), 4.25-4.17 (m, 2H), 3.75-3.74 (m, 2H), 3.58-3.52 (m, 6H), 3.51-3.46 (m, 44H), 3.37-2.91 (m, 16H), 2.20 (s, 3H), 2.29 (t, J=6.4 Hz, 2H), 2.21-2.08 (m, 4H), 2.01-1.83 (m, 6H), 1.75-1.59 (m, 6H), 1.48-1.36 (m, 6H), 1.29-1.24 (m, 3H), 1.23-1.16 (m, 4H), 0.89-0.81 (m, 9H) ppm. One proton of carboxyl group on TFA was revealed.

Drug linker PB091 can be used to make a conjugate such as PA091.

### Example 23: Preparation of the Drug-Linker (PB092) Containing a PEG Unit and a Cleavable Linker Attached to Exatecan

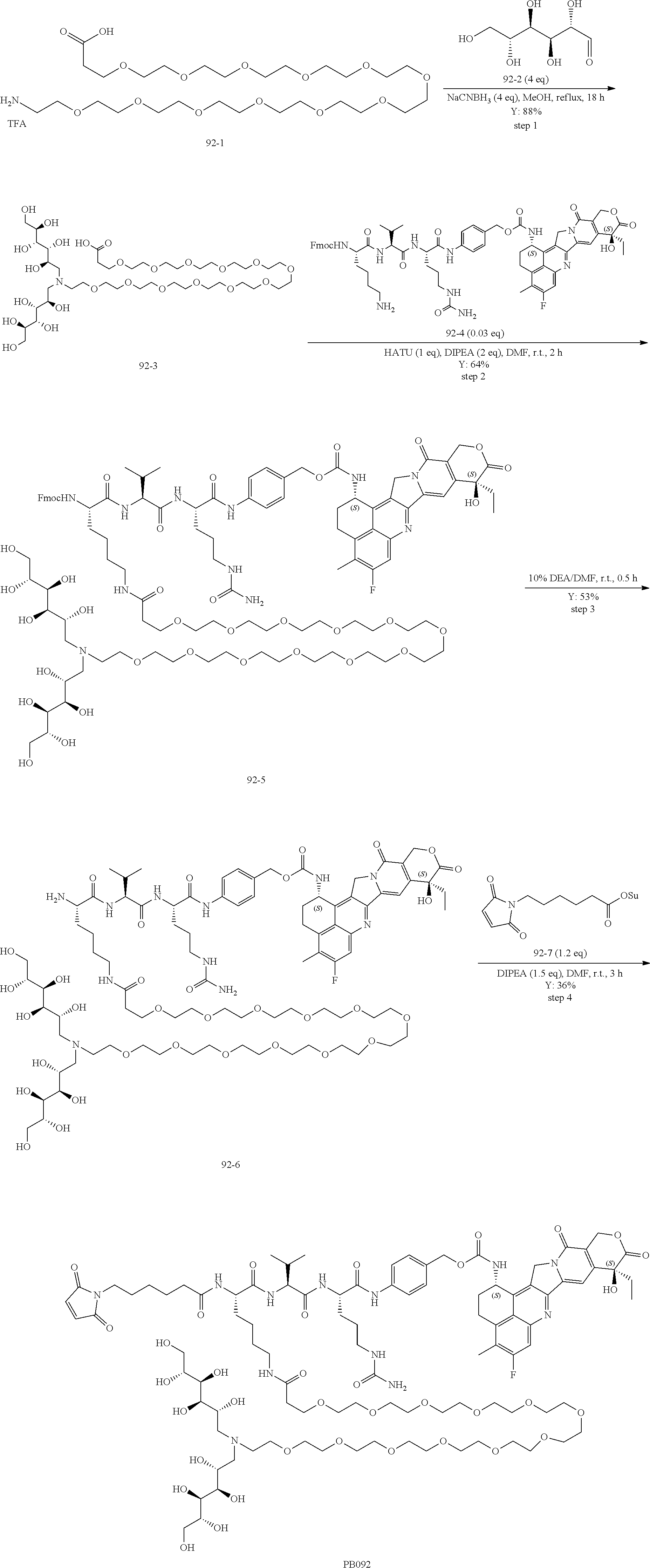

A Drug-Linker containing a PEG unit and a cleavable linker attached to exatecan (PB092) was prepared as follows:

### Step 1

A reaction mixture of compound 92-1 (1-amino-3,6,9,12,15,18,21,24,27,30,33,36-dodecaoxanonatriacontan-39-oic acid, TFA salt (92-1, 570.91 mg, 0.781 mmol)), compound 92-2 ((2S,3S,4R,5R)-2,3,4,5,6-pentahydroxyhexanal (92-2, 562.32 mg, 3.124 mmol)), and Na(CN)BH₃ (193.69 mg, 3.124 mmol) in methanol (16 mL) was heated under reflux under N₂ for 18 h until the reaction was complete as indicated by LCMS. The solvents were evaporated with a rotary evaporator, and the residue was dissolved in water and purified by reverse phase flash chromatography (0.01% TFA) to give the desired product 92-3 ((42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanoic acid (92-3, 650 mg, 0.687 mmol, 87.98%)) as a white foam. ESI m/z: 473.9 (M/2+H)⁺. 946.6 (M+H)⁺.

### Step 2

A solution of compound 92-3 ((42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanoic acid (92-3, 199.95 mg, 0.212 mmol)), compound 92-4 ((9H-fluoren-9-yl)methyl N-[(1S)-5-amino-1-{[(1S)-1-{[(1S)-4-(carbamoylamino)-1-({4-[{[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (92-4, 210 mg, 0.176 mmol)) and DIPEA (8.02 mg, 0.062 mmol) in anhydrous DMF (4 mL) was stirred at room temperature for 5 min, then a solution of HATU (67.04 mg, 0.176 mmol) in anhydrous DMF (4 mL) was added dropwise over 5 min. The resulting solution was stirred for another 2 h at room temperature until LCMS indicated complete reaction. The reaction solution was purified directly by reverse phase flash chromatography (0.01% TFA) to yield compound 92-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]pentyl]carbamate (92-5, 240 mg, 0.113 mmol, 64.22%)) as a white solid. ESI m/z=707.2 (M/2+H)⁺, 530.9 (M/3+H)⁺. ¹H NMR (400 MHz, DMSO-d6): δ 10.06 (s, 1H), 8.16 (d, J=6.4 Hz, 1H), 8.07 (d, J=8.8 Hz, 1H), 7.95 (brs, 1H), 7.88 (d, J=7.6 Hz, 2H), 7.83-7.77 (m, 2H), 7.73-7.70 (m, 3H), 7.60 (d, J=8.0 Hz, 2H), 7.55 (d, J=3.6 Hz, 1H), 7.43-7.31 (m, 7H), 6.54 (s, 1H), 5.99 (t, J=5.6 Hz, 1H), 5.50-5.34 (m, 5H), 5.34-5.24 (m, 3H), 5.08 (s, 2H), 4.67-4.23 (m, 10H), 4.07-3.96 (m, 1H), 3.96-3.83 (m, 2H), 3.83-3.76 (m, 2H), 3.65-3.44 (m, 62H), 3.24-3.11 (m, 4H), 3.11-2.89 (m, 6H), 2.38 (s, 3H), 2.29 (d, J=6.4 Hz, 2H), 2.22-2.10 (m, 2H), 2.00-1.81 (m, 3H), 1.70-1.48 (m, 4H), 1.48-1.24 (m, 7H), 0.90-0.81 (m, 9H) ppm. One proton of carboxyl group on TFA was revealed.

### Step 3

A solution of compound 92-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]pentyl]carbamate (92-5, 230 mg, 0.109 mmol)) in DMF (1 mL) was stirred at room temperature. and diethyl amine (0.11 mL, 1.068 mmol) was added. The resulting solution was stirred for 30 min to completion (monitored by LCMS). Then the diethyl amine was evaporated off and the residue in DMF was purified by reverse phase flash chromatography (0.01% TFA) to yield the expected product 92-6 ({4-[(2S)-2-[(2S)-2-[(2S)-2-amino-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (92-6, 130 mg, 0.069 mmol, 63.13%)) as a white solid. ESI m/z: 949.6 (M/2+H)⁺, 633.2 (M/3+H)⁺.

### Step 4

A solution of compound 92-6 ({4-[(2S)-2-[(2S)-2-[(2S)-2-amino-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (92-6, 50 mg, 0.026 mmol)) and compound 92-7 (2,5-dioxopyrrolidin-1-yl 6-(2,5-dioxo-2,5-dihydro-1H-pyrrol-1-yl)hexanoate (92-7, 9.74 mg, 0.032 mmol)) in anhydrous DMF (1 mL) was stirred at room temperature for 5 min, then DIPEA (5.10 mg, 0.040 mmol) was added. The resulting solution was stirred for another 3 hr until LCMS indicated the starting amine was substantially consumed. Then the resulting solution was purified directly by PrepHPLC (0.01% TFA) to yield a TFA salt of PB092 ({4-[(2S)-5-(carbamoylamino)-2-[(2S)-2-[(2S)-2-[6-(2,5-dioxo-2,5-dihydro-1H-pyrrol-1-yl)hexanamido]-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB092, 20 mg, 0.010 mmol, 36.31%)) as a white solid. ESI m/z: 697.5 (M/3+H)⁺, retention time 5.759 min (HPLC). ¹H NMR (400 MHz, DMSO-d6): δ 10.03 (s, 1H), 8.12 (d, J=7.2 Hz, 1H), 8.06 (d, J=8.8 Hz, 1H), 7.96 (d, J=8.0 Hz, 2H), 7.82-7.77 (m, 2H), 7.66 (d, J=8.8 Hz, 1H), 7.60 (d, J=8.4 Hz, 2H), 7.36 (d, J=8.4 Hz, 2H), 7.32 (s, 1H), 7.00 (s, 2H), 6.54 (s, 1H), 5.99 (t, J=6.0 Hz, 1H), 5.49-5.34 (m, 5H), 5.34-5.24 (m, 3H), 5.08 (s, 2H), 4.72-4.31 (m, 7H), 4.31-4.17 (m, 2H), 3.95-3.87 (m, 2H), 3.81-3.76 (m, 2H), 3.65-3.46 (m, 6H), 3.26-3.16 (m, 4H), 3.11-2.92 (m, 6H), 2.38 (s, 3H), 2.29 (d, J=6.4 Hz, 2H), 2.24-2.07 (m, 4H), 2.00-1.83 (m, 3H), 1.72-1.56 (m, 3H), 1.48-1.41 (m, 6H), 1.36-1.13 (m, 8H), 0.90-0.81 (m, 9H) ppm. One proton of carboxyl group on TFA was revealed. Drug linker PB092 can be used to make a conjugate such as PA092.

### Example 24: Preparation of the Drug-Linker (PB093) Containing a PEG Unit and a Cleavable Linker Attached to Exatecan

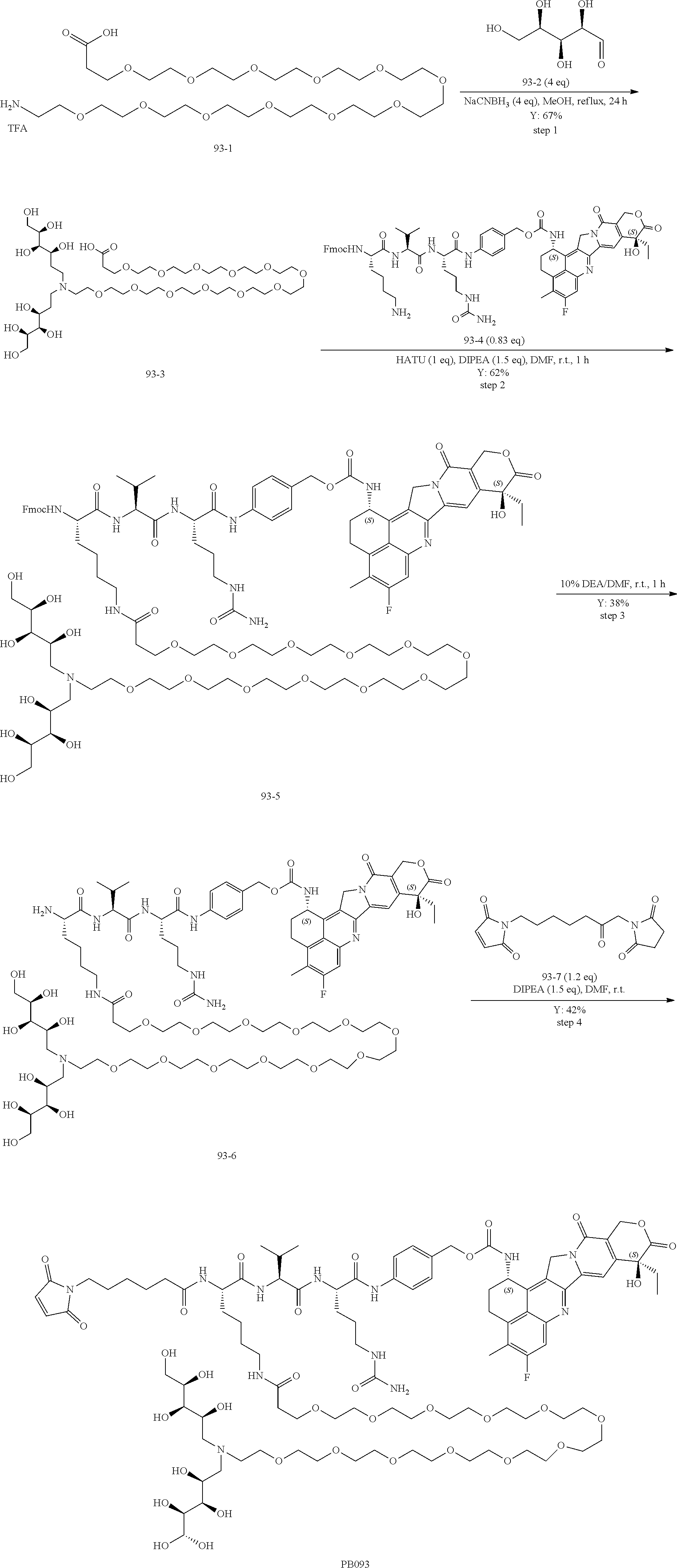

A Drug-Linker containing a PEG unit and a cleavable linker attached to exatecan (PB093) was prepared as follows:

### Step 1

A solution of compound 93-1 (1-amino-3,6,9,12,15,18,21,24,27,30,33,36-dodecaoxanonatriacontan-39-oic acid (93-1, TFA salt, 500 mg, 0.684 mmol)) in MeOH (8 mL) was treated with compound 93-2 ((2R,3S,4R)-2,3,4,5-tetrahydroxypentanal (93-2, 410.75 mg, 2.736 mmol)) and stirred at room temperature for 2 h. Then sodium cyanoborohydride (171.93 mg, 2.736 mmol) was added, and the mixture was warmed to 50° C. for 24 h. The solution was concentrated to half volume and purified by reverse phase flash chromatography (0.01% TFA) to yield product 93-3 ((42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanoic acid (93-3, 417 mg, 0.471 mmol, 68.81%)) as a white solid. ESI m/z: 886.6 (M+H)⁺.

### Step 2

To a solution of compound 93-3 ((42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanoic acid (93-3, 200 mg, 0.226 mmol)) in DMF (2 mL) were added compound 93-4 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamate (93-4, 228.59 mg, 0.192 mmol)) and DIPEA (43.76 mg, 0.339 mmol), and then a solution of HATU (85.83 mg, 0.226 mmol) in DMF (0.5 mL) was added slowly. After addition, the reaction was stirred at room temperature for another 1 h. The mixture was concentrated and purified by reverse phase flash chromatography (0.01% TFA) to afford compound 93-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}-5-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]pentyl]carbamate (93-5, 290 mg, 0.141 mmol, 62.38%)) as a white solid. ESI m/z 1030.7 (M/2+H)⁺.

### Step 3

A solution of compound 93-5 ((9H-fluoren-9-yl)methyl N-[(1S)-1-{[(1S)-1-{[(1S)-4-(carbamoylamino)-1-({4-[(I{[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]pentyl]carbamate (93-5, 290 mg, 0.141 mmol)) in DMF (2 mL) was combined with diethyl amine (0.2 mL, 1.250 mmol) and stirred at room temperature for 1 h to completion (monitored by LCMS). Then diethyl amine was removed with a rotary evaporator and the residue was purified by reverse phase flash chromatography (0.01% TFA) to yield product 93-6 ({4-[(2S)-2-[(2S)-2-[(2S)-2-amino-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (93-6, 100 mg, 0.054 mmol, 38.65%)) as a white solid. ESI m/z: 919.7 (M/2+H)⁺.

### Step 4

A solution of compound 93-6 ({4-[(2S)-2-[(2S)-2-[(2S)-2-amino-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (93-6, 110 mg, 0.060 mmol)) in DMF (1.5 mL) was stirred at room temperature, then DIPEA (0.015 mL, 0.090 mmol) and compound 93-7 (2,5-dioxopyrrolidin-1-yl 6-(2,5-dioxo-2,5-dihydro-1H-pyrrol-1-yl)hexanoate (93-7, 20.14 mg, 0.065 mmol)) were added sequentially. The resulting solution was stirred for another 1 h until LCMS showed that the reaction was completed. The completed reaction solution was purified by directly by Prep-HPLC (0.01% TFA) to yield a TFA salt of product PB093 ({4-[(2S)-5-(carbamoylamino)-2-[(2S)-2-[(2S)-2-[6-(2,5-dioxo-2,5-dihydro-1H-pyrrol-1-yl)hexanamido]-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]pentanamido]phenyl}methyl N—[(I10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB093, 51.21 mg, 0.025 mmol, 42.12%)) as a white solid. ESI m/z: 677.5 (M/3+H)⁺, retention time 5.924 min (HPLC). ¹H NMR (400 MHz, DMSO) δ 10.02 (s, 1H), 8.19 (brs, 1H), 8.12 (d, J=7.2 Hz, 1H), 8.06 (d, J=8.8 Hz, 1H), 7.96 (d, J=8.0 Hz, 1H), 7.82-7.76 (m, 2H), 7.66 (d, J=8.8 Hz, 1H), 7.61 (d, J=8.4 Hz, 2H), 7.36 (d, J=8.4 Hz, 2H), 7.32 (s, 1H), 7.00 (s, 2H), 6.53 (s, 1H), 6.00 (t, J=5.6 Hz, 1H), 5.46-5.33 (m, 6H), 5.33-5.23 (m, 3H), 5.09 (s, 2H), 4.89-4.59 (m, 6H), 4.41-4.35 (m, 1H), 4.29-4.18 (m, 2H), 4.03-3.94 (m, 2H), 3.79-3.77 (m, 2H), 3.59-3.56 (m, 8H), 3.54-3.37 (m, 52H), 3.28-3.22 (m, 2H), 3.15-2.89 (m, 6H), 2.38 (s, 3H), 2.29 (t, J=6.4 Hz, 2H), 2.24-2.08 (m, 4H), 2.01-1.82 (m, 3H), 1.73-1.56 (m, 3H), 1.53-1.43 (m, 6H), 1.43-1.36 (m, 3H), 1.33-1.16 (m, 4H), 0.94-0.74 (m, 9H). One proton of carboxyl group in TFA was revealed.

Drug linker PB093 can be used to make a conjugate such as PA093.

### Example 25: Preparation of the Drug-Linker (PB094) Containing a PEG Unit and a Cleavable Linker Attached to Exatecan

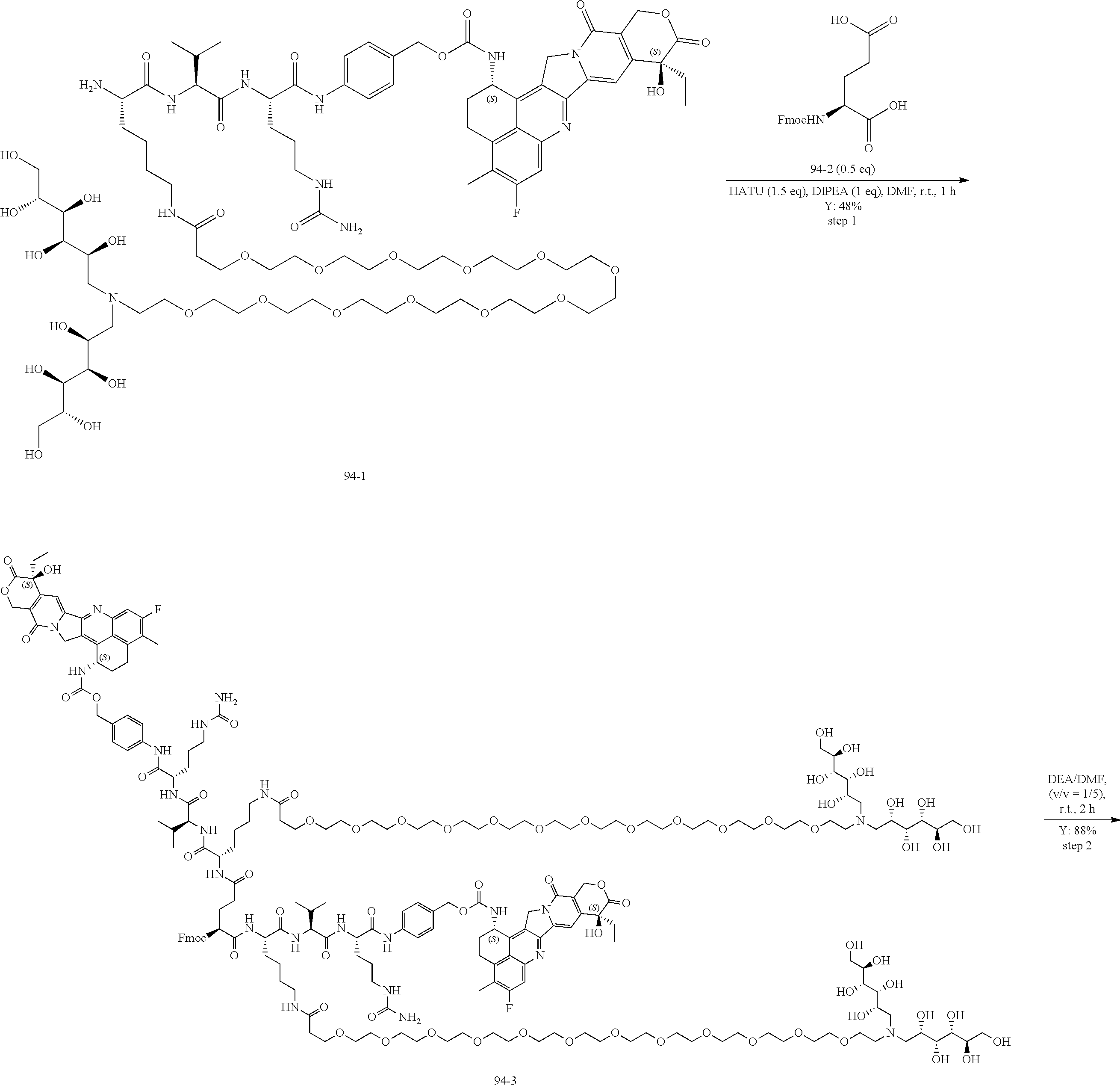

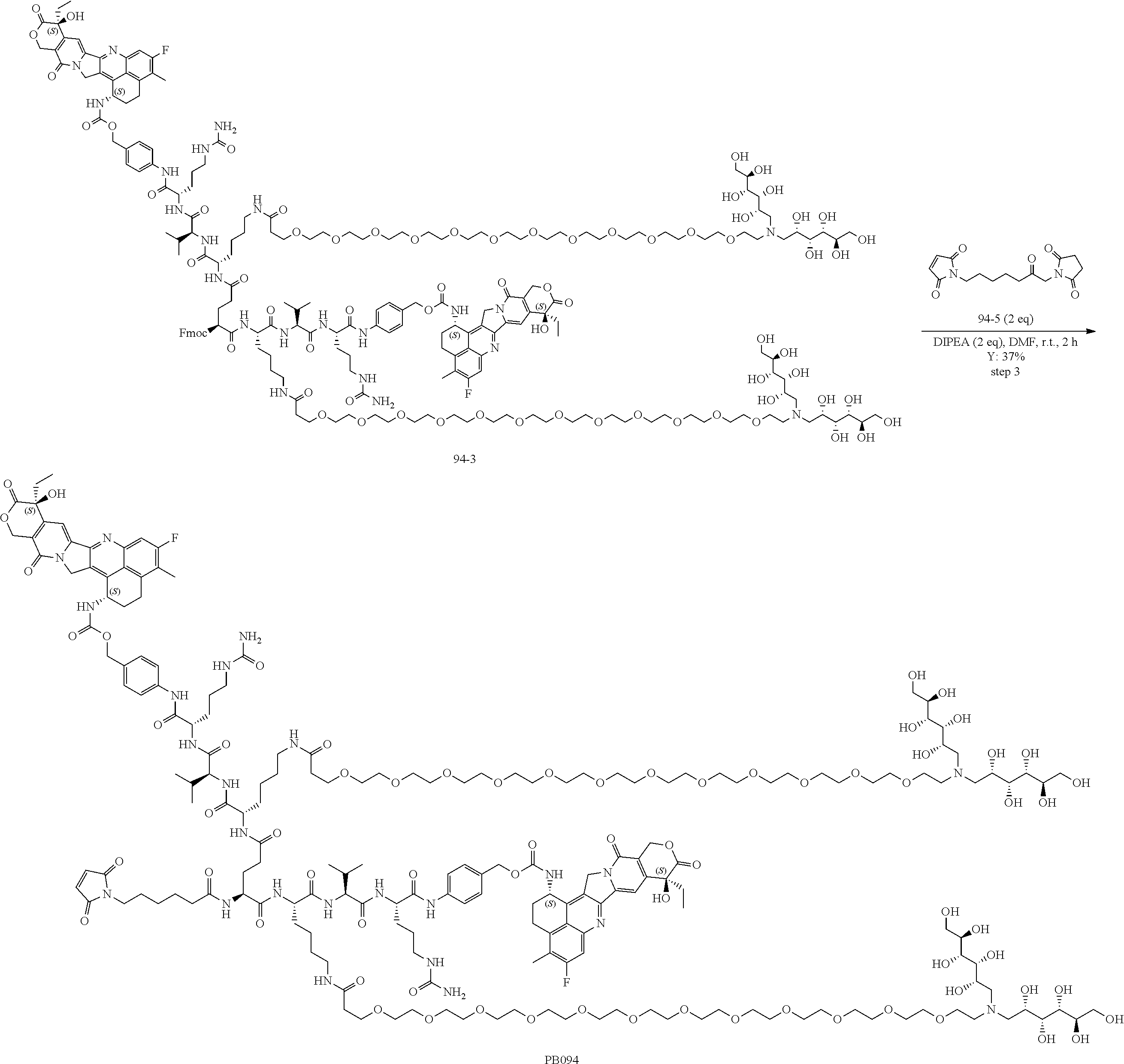

A Drug-Linker containing a PEG unit and a cleavable linker attached to exatecan (PB094) was prepared as follows:

### Step 1

To a solution of compound 94-2 ((2S)-2-({[(9H-fluoren-9-yl)methoxy]carbonyl}amino)pentanedioic acid (94-2, 19.39 mg, 0.052 mmol)) in DMF (5 mL) was added HATU (59.89 mg, 0.158 mmol) and DIPEA (13.57 mg, 0.105 mmol), followed by compound 94-1((4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenylymethyl N-[(108,238)-10-ethyl-18-fluoro-10-hydroxy-19-methyl-5,9-dioxo-8-oxa-4,15-diazahexacyclo[14.7.1.0\hat{ }{2,14}.0\hat{ }{14,13}.0\hat{ }{6,11}.0\hat{ }{20,24}]tetracosa-1,6(11),12,14,16,18,20(24)-heptaen-23-yl]carbamate (94-1, 200 mg, 0.105 mmol)). The resulting mixture was stirred at room temperature for 1 h to complete (monitored by LCMS). Then the resulting solution was purified by reverse phase flash chromatography (0.01% TFA) to afford product 94-3 ((9H-fluoren-9-yl)methyl N-[(1S)-1,3-bis({[(1S)-1-{[(1S)-1-[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl})propyl]carbamate (94-3, 108 mg, 0.026 mmol, 49.84%)) as a white solid. ESI m/z: 826.4 (M/5+H)⁺.

### Step 2

To a solution of compound 94-3 ((9H-fluoren-9-yl)methyl N-[(1S)-1,3-bis({[(1S)-1-{[(1S)-1-{[(1S)-4-(carbamoylamino)-1-({4-[(({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl})propyl]carbamate (94-3, 108 mg, 0.026 mmol)) in DMF (3 mL) was added diethyl amine (0.6 mL, 3.751 mmol). The mixture was stirred at room temperature for 2 h. The resulting solution was adjusted to pH 6 with TFA and then purified by reverse phase flash chromatography (0.01% TFA) to afford TFA salt of product 94-4 ({4-[(2S)-2-[(2S)-2-[(2S)-2-[(2S)-2-amino-4-{[(1S)-1-{[(1S)-1-{[(1S)-4-(carbamoylamino)-1-({4-[({[(10S,23S)-10-ethyl-I8-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (94-4, 90 mg, 0.023 mmol, 88.08%)) as a white solid. ESI m/z: 977.3 (M/4+H)⁺.

### Step 3

To a solution of compound 94-4 ({4-[(2S)-2-[(2S)-2-[(2S)-2-[(2S)-2-amino-4-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (94-4, 90 mg, 0.023 mmol)) in DMF (3 mL) was added DIPEA (5.96 mg, 0.046 mmol) and 2,5-dioxopyrrolidin-1-yl 6-(2,5-dioxo-2,5-dihydro-1H-pyrrol-1-yl)hexanoate (14.21 mg, 0.046 mmol). The mixture was stirred at room temperature for 2 h. The resulting solution was adjusted to pH 6 and purified by Prep-HPLC (0.01% TFA) to afford the product PB094 ({4-[(2S)-5-(carbamoylamino)-2-[(2S)-2-[(2S)-2-[(2S)-4-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-2-[6-(2,5-dioxo-2,5-dihydro-1H-pyrrol-1-yl)hexanamido]butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB094, 35 mg, 0.009 mmol, 37.06%)) as a white solid. ESI m/z: 820.7 (M+H)⁺, 1025.5 (M+H)⁺, retention time 5.452 min (HPLC). ¹H NMR (400 MHz, DMSO-d6) δ 10.04 (s, 2H), 8.19-8.15 (m, 4H), 8.14-8.12 (m, 4H), 8.07-8.05 (m, 1H), 7.97 (d, J=8.0 Hz, 1H), 7.89-7.82 (m, 3H), 7.76 (d, J=10.8 Hz, 2H), 7.62-7.60 (m, 4H), 7.37-7.35 (m, 4H), 7.31 (s, 2H), 7.00 (s, 2H), 6.53 (s, 2H), 6.00 (t, J=6.6 Hz, 2H), 5.51-5.44 (m, 12H), 5.34-5.21 (m, 6H), 5.08 (s, 4H), 4.88-4.70 (m, 4H), 4.55-4.43 (m, 8H), 4.38-4.33 (m, 3H), 4.29-4.25 (m, 2H), 4.20-4.14 (m, 3H), 4.01-3.99 (m, 4H), 3.79-3.78 (m, 4H), 3.69-3.66 (m, 4H), 3.62-3.56 (m, 18H), 3.53-3.48 (m, 88H), 3.46-3.42 (m, 10H), 3.27-3.12 (m, 7H), 3.12-2.90 (m, 12H), 2.37 (s, 6H), 2.31-2.27 (m, 4H), 2.21-2.01 (m, 8H), 1.95-1.79 (m, 8H), 1.73-1.55 (m, 8H), 1.49-1.40 (m, 8H), 1.40-1.31 (m, 6H), 1.31-1.14 (m, 6H), 0.89-0.82 (m, 18H) ppm. Two protons of carboxyl group in TFA were revealed.

Drug linker PB094 can be used to make a conjugate such as PA094.

### Example 26: Preparation of the Drug-Linker (PB095) Containing a PEG Unit and a Cleavable Linker Attached to MMAE

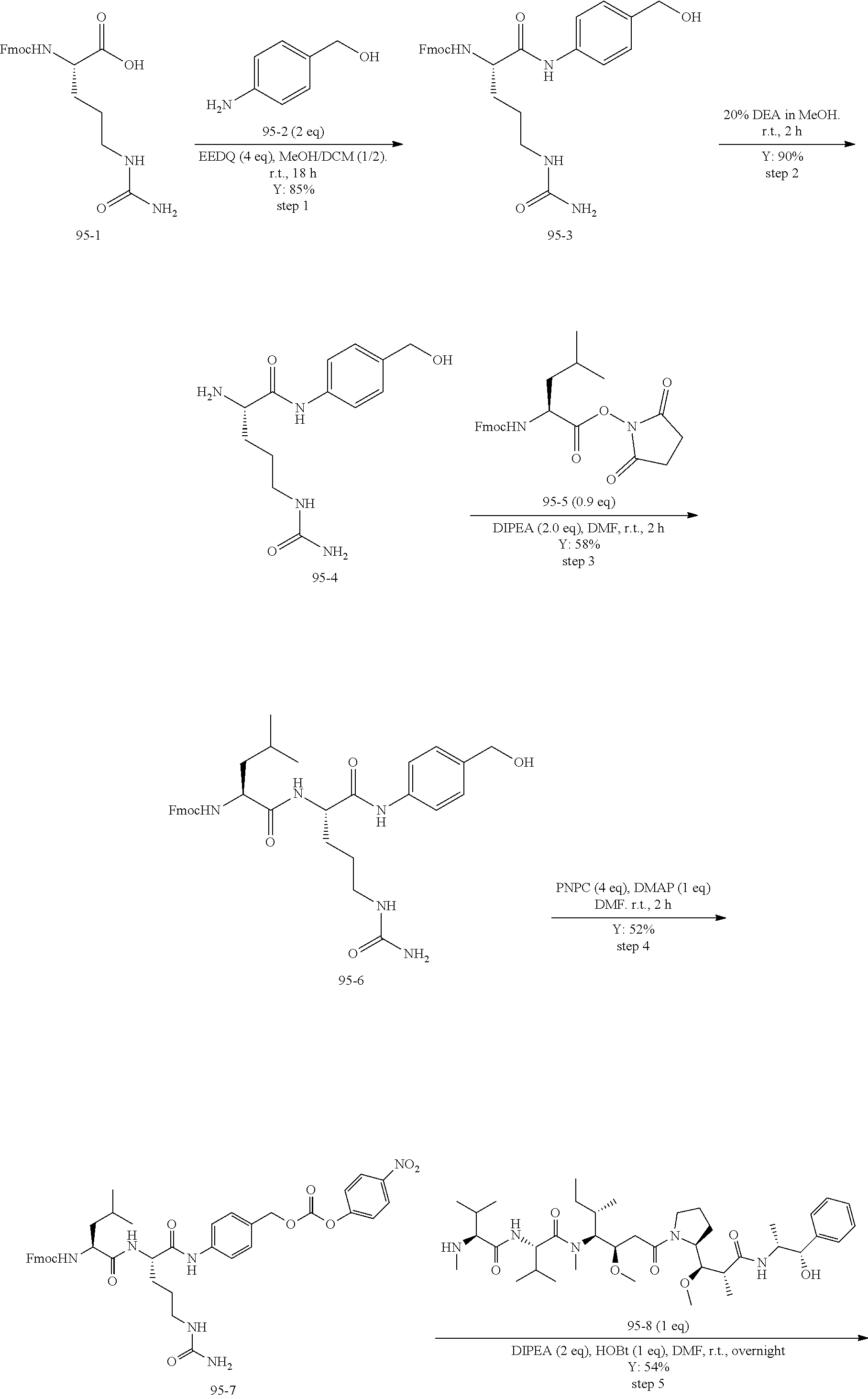

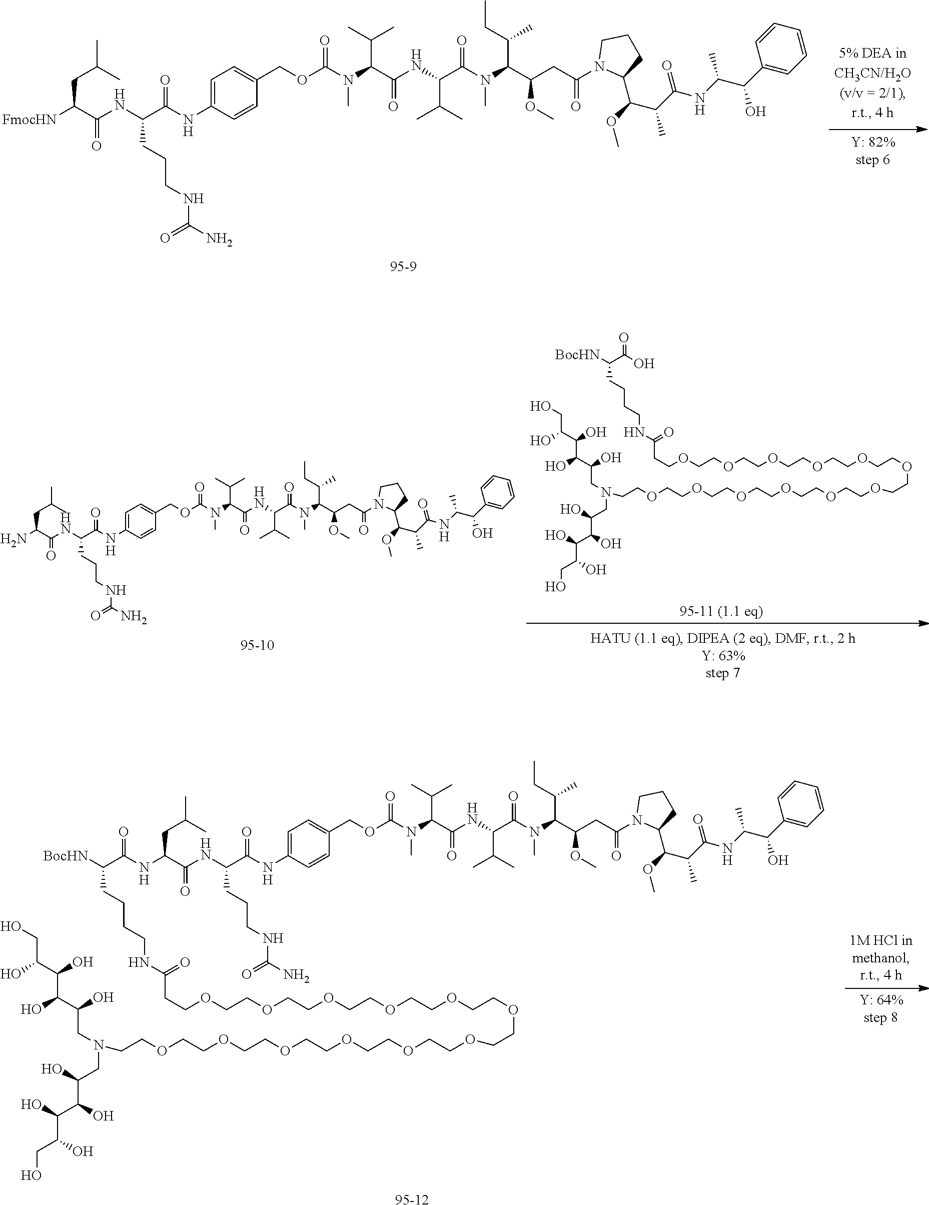

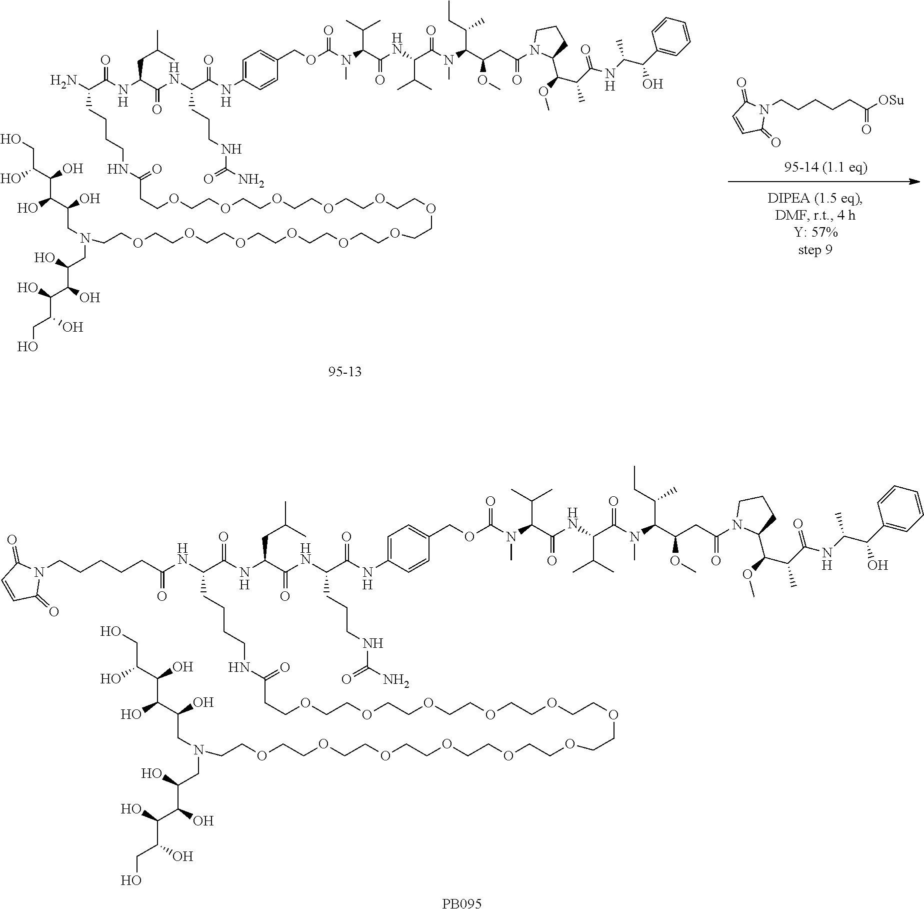

A Drug-Linker containing a PEG unit and a cleavable linker attached to MMAE (PB095) was prepared as follows:

### Step 1

A solution of compound 95-1 (2S)-5-(carbamoylamino)-2-({[(9H-fluoren-9-yl)methoxy]carbonyl}amino)pentanoic acid (95-1, 6.0 g, 15.097 mmol), compound 95-2 ((4-aminophenyl)methanol (95-2, 3.72 g, 30.195 mmol)) and EEDQ (14.93 g, 60.390 mmol) in MeOH (25 mL) and DCM (50 mL) was stirred at room temperature for 18 h and LCMS showed that the reaction was completed. The reaction solution was concentrated to dryness and then purified by reverse phase flash chromatography (0.01% TFA) to yield the desired fractions, which were freeze-dried to yield compound 95-3 ((9H-fluoren-9-yl)methyl N-[(1S)-4-(carbamoylamino)-1-{[4-(hydroxymethyl)phenyl]carbamoyl}butyl]carbamate (95-3, 6.45 g, 12.834 mmol, 85.01%)) as a white solid. ESI m/z: 503.3 (M+H)⁺.

### Step 2

To a solution of compound 95-3 ((9H-fluoren-9-yl)methyl N-[(1S)-4-(carbamoylamino)-1-{[4-(hydroxymethyl)phenyl]carbamoyl}butyl]carbamate (95-3, 6.45 g, 12.834 mmol)) in MeOH (20 mL) was added diethyl amine (5 mL, 31.260 mmol). The mixture was stirred at room temperature for 2 h to achieve complete deprotection. The solution was concentrated and purified by reverse phase flash chromatography (0.01% TFA) to yield product 95-4 ((2S)-2-amino-5-(carbamoylamino)-N-[4-(hydroxymethyl)phenyl]pentanamide (95-4, 3.25 g, 11.594 mmol, 90.34%)) as a pale yellow solid. ESI m/z: 281.3 (M+H)⁺.

### Step 3

A solution of compound 95-4 ((2S)-2-amino-5-(carbamoylamino)-N-[4-(hydroxymethyl)phenyl]pentanamide (95-4, 3.76 g, 13.413 mmol)), compound 95-5 (2,5-dioxopyrrolidin-1-yl (2S)-2-({[(9H-fluoren-9-yl)methoxy]carbonyl}amino)-4-methylpentanoate (95-5, 6.65 g, 14.755 mmol)) and DIPEA (3.47 g, 26.826 mmol) in DMF (10 mL) was stirred at room temperature for 2 h to completion. The reaction mixture was purified by reverse phase flash chromatography (0.01% TFA) to yield the desired fractions, which were freeze-dried to yield compound 95-6 ((9H-fluoren-9-yl)methyl N-[(1S)-1-{[(1S)-4-(carbamoylamino)-1-{[4-(hydroxymethyl)phenyl]carbamoyl}butyl]carbamoyl}-3-methylbutyl]carbamate (95-6, 4.8 g, 7.796 mmol, 58.12%)) as a white solid. ESI m/z: 616.3 (M+H)⁺. ¹H NMR (400 MHz, DMSO) δ 9.97 (s, 1H), 8.05 (d, J=8.0 Hz, 1H), 7.89 (d, J=7.2 Hz, 2H), 7.74-7.70 (m, 2H), 7.55-7.51 (m, 3H), 7.44-7.39 (m, 2H), 7.34-7.30 (m, 2H), 7.23 (d, J=8.4 Hz, 2H), 5.97 (t, J=5.6 Hz, 1H), 5.41 (s, 2H), 5.10 (t, J=5.6 Hz, 1H), 4.43-4.39 (m, 3H), 4.32-4.20 (m, 3H), 4.15-4.05 (m, 1H), 3.07-2.89 (m, 2H), 1.74-1.55 (m, 3H), 1.51-1.32 (m, 4H), 0.90-0.83 (m, 6H) ppm.

### Step 4

A solution of compound 95-6 ((9H-fluoren-9-yl)methyl N-[(1S)-1-{[(1S)-4-(carbamoylamino)-1-{[4-(hydroxymethyl)phenyl]carbamoyl}butyl]carbamoyl}-3-methylbutyl]carbamate (95-6, 2.0 g, 3.248 mmol)), bis(4-nitrophenyl) carbonate (3.95 g, 12.993 mmol) and DMAP (0.40 g, 3.248 mmol) in DMF (5 mL) was stirred at room temperature for 2 h. Then the reaction mixture was quenched with drops of water and purified by reverse phase flash chromatography (neutral eluent) to yield the desired fractions, which were freeze-dried to yield compound 95-7 ({4-[(2S)-5-(carbamoylamino)-2-[(2S)-2-({[(9H-fluoren-9-yl)methoxy]carbonyl}amino)-4-methylpentanamido]pentanamido]phenyl}methyl 4-nitrophenyl carbonate (95-7, 1.33 g, 1.703 mmol, 52.44%)) as a pale yellow solid. ESI m/z: 781.3 (M+H)⁺.

### Step 5

A solution of compound 95-7 ({4-[(2S)-5-(carbamoylamino)-2-[(2S)-2-({[(9H-fluoren-9-yl)methoxy]carbonyl}amino)-4-methylpentanamido]pentanamido]phenyl}methyl 4-nitrophenyl carbonate (95-7, 340 mg, 0.435 mmol)), compound 95-8 ((2S)—N-[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]-3-methyl-2-(methylamino)butanamide (95-8, 312.63 mg, 0.435 mmol)) and HOBt (58.84 mg, 0.435 mmol) in anhydrous DMF (4 mL) was stirred at room temperature for 5 min, then DIPEA (112.34 mg, 0.871 mmol) was added. The resulting yellow solution was stirred for overnight until LCMS indicated both starting materials were substantially consumed. The resulting solution was purified directly by reverse phase flash chromatography (0.01% TFA) to yield compound 95-9 ((9H-fluoren-9-yl)methyl N-[(1S)-1-{[(1S)-4-(carbamoylamino)-1-({4-({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-3-methylbutyl]carbamate (95-9, 320 mg, 0.235 mmol, 54.10%)) as a white solid. ESI m/z: 680.6 (M/2+H)⁺; 718.7 (fragment piece of MMAE); 598.4 (fragment piece of Linker Fmoc-Leu-Cit-PAB).

### Step 6

A solution of compound 95-9 ((9H-fluoren-9-yl)methyl N-[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1- phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-3-methylbutyl]carbamate (95-9, 320 mg, 0.235 mmol)) in CH₃CN (10 mL) and water (5 mL) was stirred at room temperature and diethyl amine (0.8 mL, 7.766 mmol) was added. The resulting solution was stirred for 4 h to complete. Diethyl amine was evaporated off under vacuo and the residue was purified by reverse phase flash chromatography (0.01% TFA) to yield a TFA salt of product 95-10 ({4-[(2S)-2-[(2S)-2-amino-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-10, 220 mg, 0.193 mmol, 82.18%)) as a white solid and the product was used for next step without any purification. ESI m/z: 569.5 (M/2+H)⁺.

¹H NMR (400 MHz, DMSO-d6) δ 10.21 (s, 1H), 8.79 (d, J=7.6 Hz, 1H), 8.31-8.06 (m, 4H), 7.91 (d, J=8.8 Hz, 0.5H), 7.65 (d, J=8.4 Hz, 0.5H), 7.60-7.57 (m, 2H), 7.37-7.24 (m, 6H), 7.20-7.14 (m, 1H), 6.08 (s, 1H), 5.60-5.31 (m, 2H), 5.12-4.98 (m, 2H), 4.74-4.62 (m, 1H), 4.55-4.40 (m, 3H), 4.30-4.23 (m, 1H), 4.05-3.92 (m, 2H), 3.85-3.76 (m, 2H), 3.58-3.55 (m, 2H), 3.32-3.12 (m, 9H), 3.08-2.97 (m, 4H), 2.89-2.83 (m, 3H), 2.44-2.39 (m, 1H), 2.29-2.23 (m, 1H), 2.16-1.92 (m, 3H), 1.84-1.39 (m, 13H), 1.31-1.25 (m, 1H), 1.06-0.97 (m, 6H), 0.92-0.75 (m, 24H) ppm. One proton of carboxyl group in TFA is revealed.

### Step 7

A solution of compound 95-10 ({4-[(2S)-2-[(2S)-2-amino-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-10, 218 mg, 0.192 mmol)), compound 95-11 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (95-11, 247.57 mg, 0.211 mmol)) and DIPEA (49.45 mg, 0.383 mmol) in anhydrous DMF (3 mL) was stirred at room temperature for 5 min to ensure the starting acid dissolve completely, and then a solution of HATU (80.16 mg, 0.211 mmol) in anhydrous DMF (1 mL) was added slowly. The resulting solution was stirred for another 2 h until LCMS indicated a complete reaction. Then the reaction solution was purified directly by reverse phase flash chromatography (0.01% TFA) to give compound 95-12 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-12, 280 mg, 0.122 mmol, 63.69%)) as a white solid. ESI m/z: 765.5 (M/3+H)⁺.

### Step 8

A solution of compound 95-12 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-12, 260 mg, 0.113 mmol)) in methanol was stirred at room temperature, and 3M HCl in MeOH (2 mL) was added slowly. The resulting light yellow solution was kept stirring for another 4 h until complete deprotection was achieved (monitored by LCMS). Then the solution was concentrated with a rotary evaporator to remove solvent, the residue was dissolved with water, and purified by reverse phase flash chromatography (0.01% TFA) to yield compound 95-13 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]—N-methylcarbamate (95-13, 160 mg, 0.073 mmol, 64.35%)) as a white solid. ESI m/z: 732.2 (M/3+H)⁺, 718.6 (fragment MMAE), 478.2 ((Linker_{Boc-Lys(PEG12-sugar)-Leu-Cit-pab}-18)/3+H)⁺.

### Step 9

A solution of compound 95-13 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-13, 80 mg, 0.036 mmol)) and DIPEA (7.06 mg, 0.055 mmol) in anhydrous DMF (1.5 mL) was stirred at room temperature for 5 min to dissolve all materials. Then a solution of 2,5-dioxopyrrolidin-1-yl 6-(2,5-dioxo-2,5-dihydro-1H-pyrrol-1-yl)hexanoate (95-14, 12.36 mg, 0.040 mmol) in anhydrous DMF (0.5 mL) was added dropwise by syringe over 5 min. After addition, the resulting solution was stirred for another 4 h until LCMS indicated all starting amine was consumed. The resulting solution was then purified directly by Prep-HPLC (0.01% TFA) to yield the desired fractions, which were lyophilized by LabConc to yield a TFA salt of PB095 ({4-[(2S)-5-(carbamoylamino)-2-[(2S)-2-[(2S)-2-[6-(2,5-dioxo-2,5-dihydro-1H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB095, 50 mg, 0.021 mmol, 57.44%)) as a white solid. ESI m/z: 542.5 ((linker fragment, (M−717−26−18)₃+H)⁺; 796.6 (M/3+H)⁺. Retention time 6.250 min (HPLC). ¹H NMR (400 MHz, DMSO-d6) δ 10.00 (s, 1H), 8.34-8.07 (m, 1.5H), 8.00 (d, J=7.6 Hz, 1H), 7.93-7.88 (m, 2.5H), 7.82-7.79 (m, 1H), 7.65-7.57 (m, 2.5H), 7.35-7.23 (m, 6H), 7.18-7.10 (m, 1.5H), 7.00 (s, 2H), 5.99-5.98 (m, 1H), 5.53-5.25 (m, 4H), 5.14-4.95 (m, 2H), 4.83-4.17 (m, 11H), 4.04-3.92 (m, 4H), 3.79-3.77 (m, 2H), 3.70-3.66 (m, 2H), 3.61-3.56 (m, 8H), 3.53-3.45 (m, 44H), 3.25-3.12 (m, 14H), 3.05-2.83 (m, 14H), 2.44-2.39 (m, 1H), 2.31-2.14 (m, 4H), 2.14-1.96 (m, 6H), 1.85-1.44 (m, 18H), 1.36-1.16 (m, 10H), 1.06-0.97 (m, 6H), 0.89-0.75 (m, 27H) ppm. One proton of carboxyl group in TFA was revealed. Drug linker PB095 can be used to make a conjugate such as PA095.

### Example 27: Preparation of the Drug-Linker (PB096) Containing a PEG Unit and a Cleavable Linker Attached to MMAE

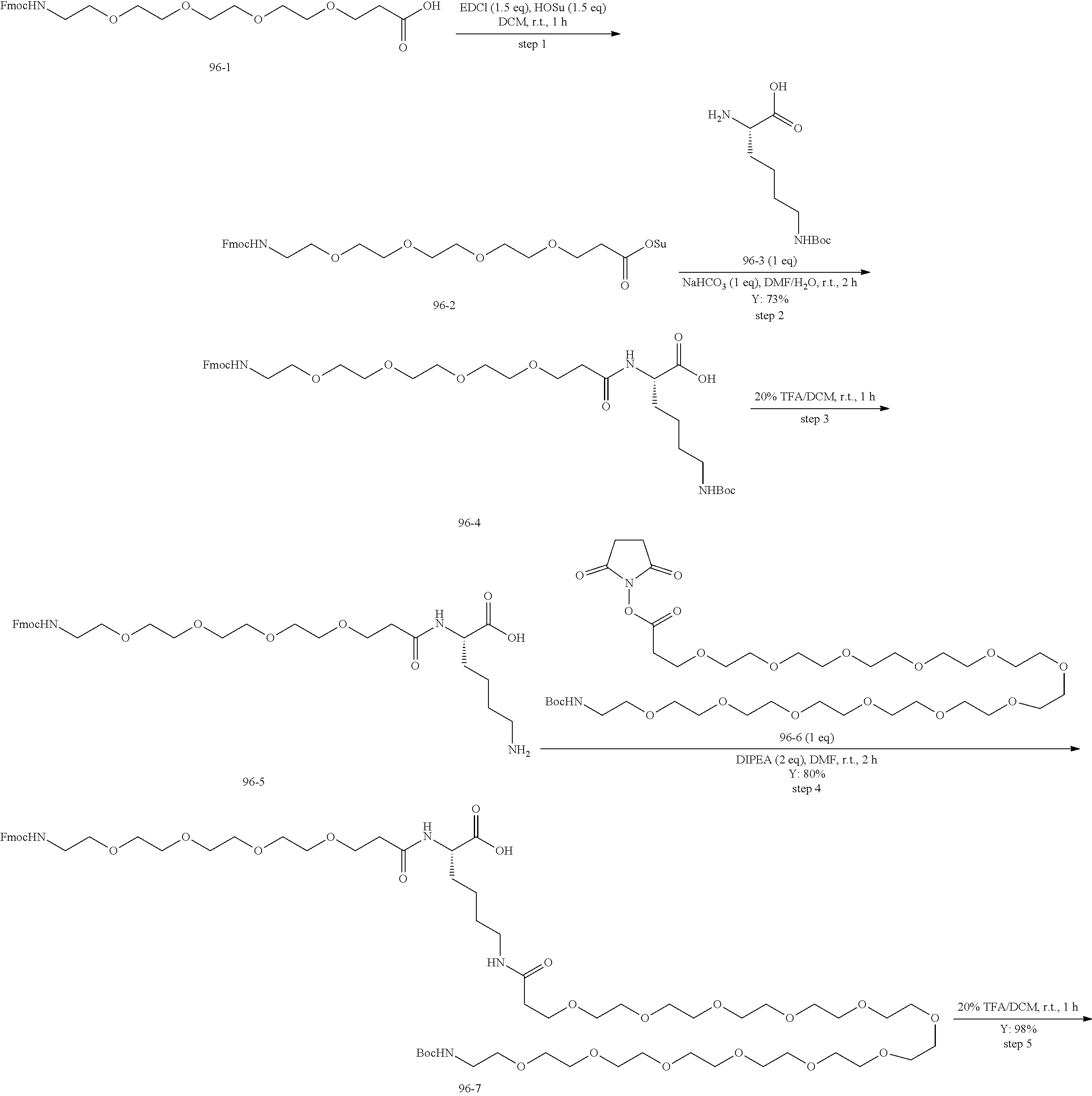

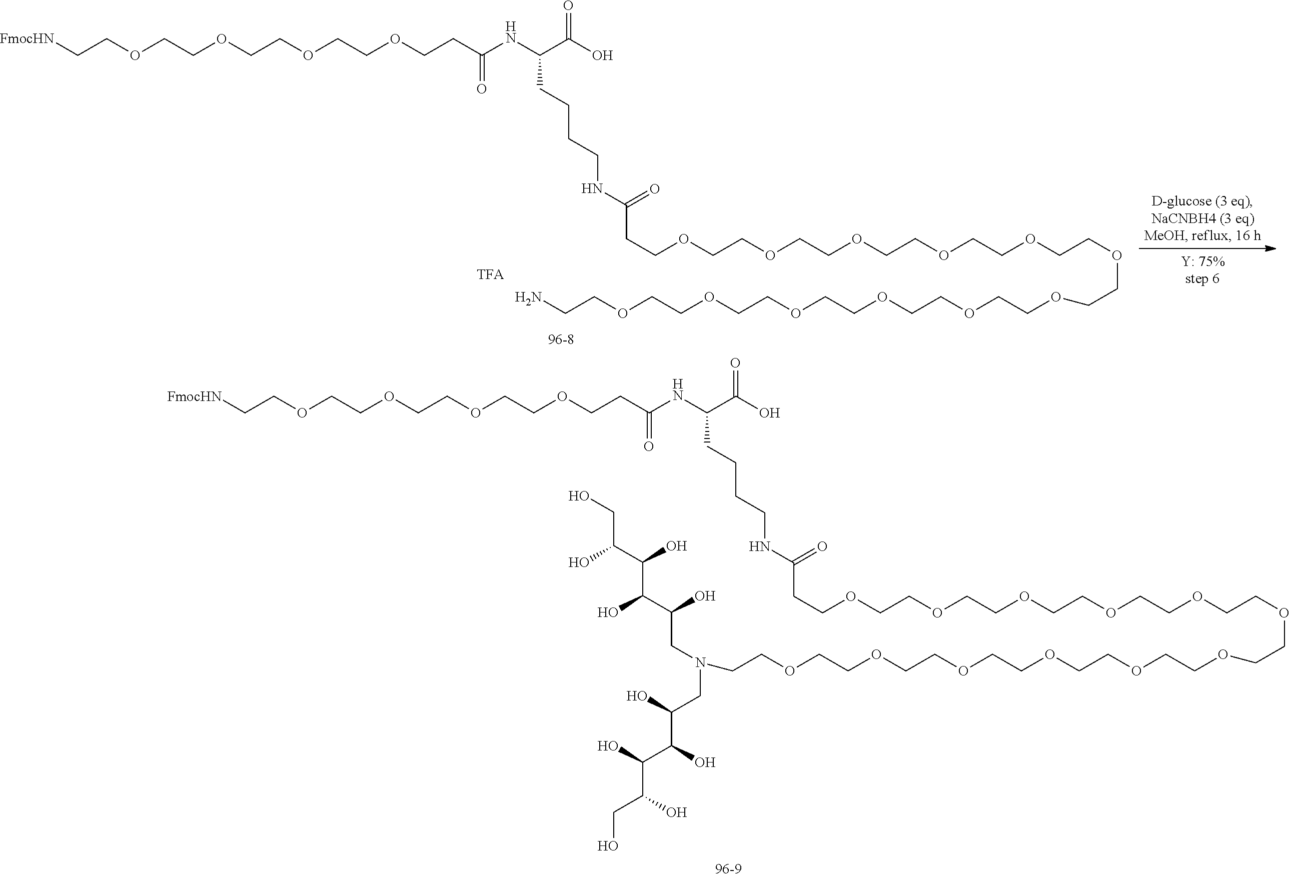

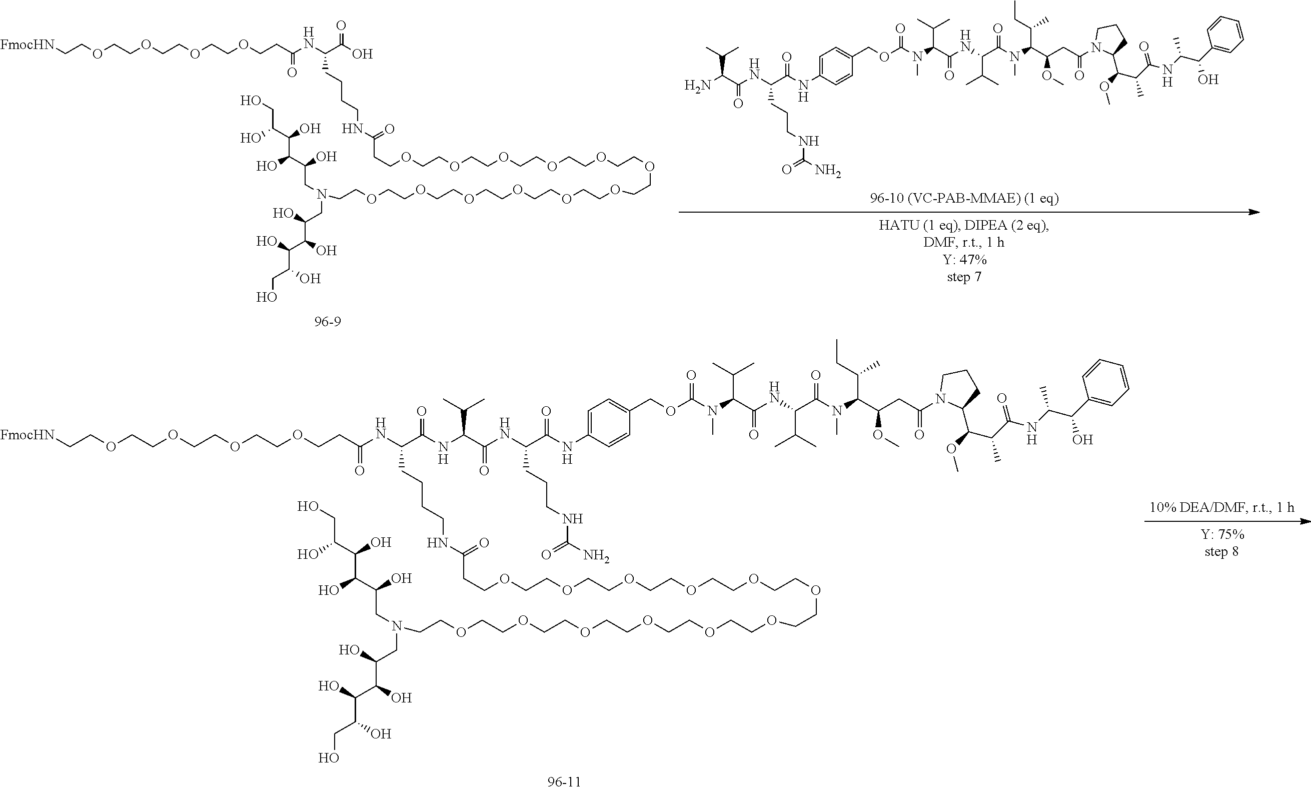

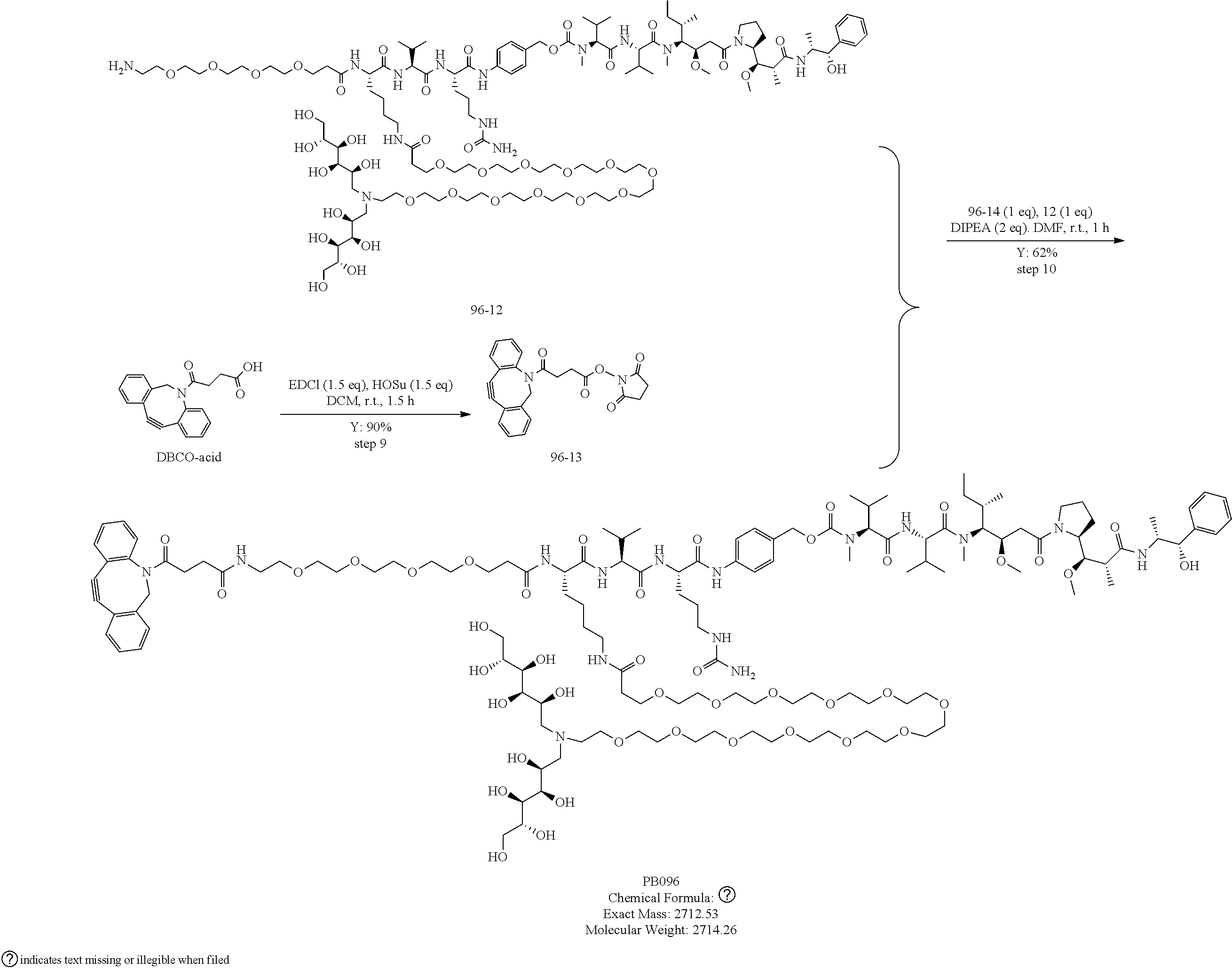

A Drug-Linker containing a PEG unit and a cleavable linker attached to MMAE (PB096) was prepared as follows:

### Step 1

A clear solution of compound 96-1 (1-({[(9H-fluoren-9-yl)methoxy]carbonyl}amino)-3,6,9,12-tetraoxapentadecan-15-oic acid (96-1, 5.00 g, 10.267 mmol)) and HOSu (1.77 g, 15.401 mmol) in dry DCM (40 mL) was stirred at room temperature and EDCI (2.95 g, 15.401 mmol) was added. The solution was kept stirring for 1 h until complete conversion was achieved. Then the solution was diluted with more DCM (20 mL) and washed with water (50 mL), the organic layer was separated and the water layer was extracted with DCM (50 mL*2). The combined collected DCM phase was dried over sodium sulfate and filtered, then concentrated under reduced pressure to yield crude material as a colorless oil compound 96-2, which was used as such in the next step. ESI m/z=585.3 (M+H)⁺.

### Step 2

A suspension of compound 96-3 (N6-(tert-butoxycarbonyl)-L-lysine (96-3, 2.233 mL, 10.267 mmol)) in DMF (12 mL) was stirred at room temperature, then a solution of sodium bicarbonate (0.86 g, 10.267 mmol) in water (3 mL) was added. The suspension was stirred for 20 min until most of starting acid was dissolved in the solvent. Then compound 96-2 (2,5-dioxopyrrolidin-1-yl 1-(9H-fluoren-9-yl)-3-oxo-2,7,10,13,16-pentaoxa-4-azanonadecan-19-oate (96-2, 6.00 g, 10.267 mmol)) was added. The resulting solution was stirred at room temperature for 2 h. The completed reaction solution was purified directly by reverse phase column chromatography (0.01% TFA) to yield compound 96-4 (N2-(1-(9H-fluoren-9-yl)-3-oxo-2,7,10,13,16-pentaoxa-4-azanonadecan-19-oyl)-N6-(tert-butoxycarbonyl)-L-lysine (96-4, 5.40 g, 7.552 mmol, 73.56%)) as a white solid. ESI m/z: 716.5 (M+H)⁺, 738.4 (M+Na)⁺. ¹H NMR (400 MHz, 400 MHz) δ 7.89 (d, J=7.6 Hz, 2H), 7.86-7.77 (m, 1H), 7.69 (d, J=7.2 Hz, 2H), 7.44-7.38 (m, 2H), 7.37-7.31 (m, 2H), 6.75 (t, J=5.6 Hz, 1H), 4.30-4.19 (m, 2H), 4.04-3.99 (m, 1H), 3.59-3.54 (m, 2H), 3.50-3.47 (m, 12H), 3.43-3.39 (m, 2H), 3.16-3.11 (m, 2H), 2.88-2.83 (m, 2H), 2.40-2.32 (m, 2H), 1.67-1.63 (m, 1H), 1.55-1.48 (m, 1H), 1.36 (s, 9H), 1.33-1.29 (m, 2H), 1.25-1.20 (m, 2H), 0.99 (d, J=6.4 Hz, 2H) ppm. Carboxyl group is not revealed.

### Step 3

A solution of compound 96-4 ((2S)-6-{[(tert-butoxy)carbonyl]amino}-2-[1-({[(9H-fluoren-9-yl)methoxy]carbonyl}amino)-3,6,9,12-tetraoxapentadecan-15-amido]hexanoic acid (96-4, 5.4 g, 7.544 mmol)) in DCM (16 mL) was stirred at room temperature and then TFA (4 mL, 53.850 mmol) was added. The resulting yellow solution was stirred for another 1 h. Then TFA and solvent were evaporated, the residue was dissolved in DCM again and concentrated to dryness. The process was repeated three times, and a crude TFA salt of product 96-5 ((1-(9H-fluoren-9-yl)-3-oxo-2,7,10,13,16-pentaoxa-4-azanonadecan-19-oyl)-L-lysine (96-5)) was obtained as a white solid. ESI m/z: 616.4 (M+H)⁺.

### Step 4

A solution of 1-{[(tert-butoxy)carbonyl]amino}-3,6,9,12,15,18,21,24,27,30,33,36-dodecaoxanonatriacontan-39-oic acid (1.58 g, 2.201 mmol) and HOSu (0.53 g, 4.597 mmol) in anhydrous DCM (20 mL) was stirred at room temperature for 5 min, then EDCI (0.63 g, 3.302 mmol) was added. The resulting solution was stirred for another 1 h, then was diluted with more DCM (20 mL) and washed with water (20 mL). The organic layer was collected and the water layer was extracted with DCM (40 mL*2). The combined DCM layer was dried over sodium sulfate, filtered and concentrated the filtrate to give crude product 96-6 (2,5-dioxopyrrolidin-1-yl 2,2-dimethyl-4-oxo-3,8,11,14,17,20,23,26,29,32,35,38,41-tridecaoxa-5-azatetratetracontan-44-oate (96-6, 2.5 g, 3.069 mmol, 100.15%)) as colorless oil. ESI m/z: 715.5 (M−100+H)⁺, 837.5 (M+Na)⁺.

A solution of compound 96-5 ((1-(9H-fluoren-9-yl)-3-oxo-2,7,10,13,16-pentaoxa-4-azanonadecan-19-oyl)-L-lysine (96-5, 1.55 g, 2.514 mmol)) and compound 96-6 (2,5-dioxopyrrolidin-1-yl 2,2-dimethyl-4-oxo-3,8,11,14,17,20,23,26,29,32,35,38,41-tridecaoxa-5-azatetratetracontan-44-oate (96-6, 1.8 g, 2.211 mmol)) in anhydrous DMF (22 mL) was stirred at room temperature for 5 min, then DIPEA (0.57 g, 4.422 mmol) was added slowly by syringe. The resulting solution was stirred for 2 h until LCMS indicated all starting amine was consumed. The resulting solution was concentrated under reduced pressure and the residue was purified directly by Prep-HPLC (0.01% TFA) to yield compound 96-7 ((2S)-6-(1-{[(tert-butoxy)carbonyl]amino}-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-7, 2.3 g, 1.748 mmol, 79.04%)) as colorless oil. ESI m/z: 608.5 ((M−100)/2+H)⁺.

### Step 5

A solution of compound 96-7 ((2S)-6-(1-{[(tert-butoxy)carbonyl]amino}-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-7, 2.3 g, 1.748 mmol)) in DCM (16 mL) was stirred at r.t., then TFA (4 mL, 53.850 mmol) was added. The resulting solution was stirred for 1 h to achieve complete deprotection. Then TFA and DCM were evaporated off, the residue was purified by reverse phase flash chromatography (0.01% TFA) to give the expected fractions, which were lyophilized to yield product 96-8 ((2S)-6-(1-amino-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-8, 2.1 g, 1.728 mmol, 99.06%)) as color less oil. ESI m/z: 608.5 (M+H)⁺. ¹H NMR (400 MHz, DMSO-d6) δ 7.89 (d, J=7.6 Hz, 2H), 7.86-7.80 (m, 2H), 7.71-7.61 (m, 1H), 7.44-7.40 (m, 2H), 7.37-7.33 (m, 2H), 6.29 (s, 1H), 4.30-4.21 (m, 1H), 4.06-3.95 (m, 2H), 3.68-3.55 (m, 12H), 3.55-3.47 (m, 48H), 3.42-3.38 (m, 2H), 3.38-3.21 (m, 4H), 3.16-3.05 (m, 2H), 3.00-2.94 (m, 4H), 2.43-2.37 (m, 2H), 2.33-2.27 (m, 2H), 1.70-1.57 (m, 1H), 1.58-1.44 (m, 1H), 1.38-1.18 (m, 4H) ppm. Two protons in amino group and two protons in carboxyl group are not revealed.

### Step 6

A suspension of compound 96-8 ((2S)-6-(1-amino-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-8, 2.0 g, 1.645 mmol)) and D-glucose (1.78 g, 9.873 mmol) in methanol (32 mL) was heated to 50° C. under nitrogen atmosphere and then sodium cyanoborohydride (0.62 g, 9.873 mmol) was added. The resulting mixture was heated overnight (16 h) until LCMS indicated complete reaction. The solution was then concentrated to dryness, and the residue was dissolved in water and purified by reverse phase flash chromatography (0.01% TFA) to give the desired fractions, which were freeze-dried over LabConco to yield compound 96-9 ((2S)-2-[1-({[(9H-fluoren-9-yl)methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (96-9, 2.0 g, 1.296 mmol, 78.74%)) as colorless oil. ESI m/z: 515.5 (M/3+H)⁺, 772.6 (M/2+H)⁺. ¹H NMR (400 MHz, DMSO-d6) δ 8.02 (d, J=7.2 Hz, 1H), 7.89 (d, J=7.2 Hz, 2H), 7.83 (t, J=5.6 Hz, 1H), 7.69 (d, J=7.2 Hz, 2H), 7.44-7.39 (m, 2H), 7.35-7.31 (m, 3H), 4.76-4.37 (m, 4H), 4.30-4.28 (m, 2H), 4.23-4.19 (m, 1H), 4.15-4.09 (m, 1H), 4.03-3.91 (m, 1H), 3.75-3.64 (m, 2H), 3.59-3.56 (m, 6H), 3.56-3.46 (m, 56H), 3.41-3.17 (m, 18H), 3.17-3.12 (m, 2H), 3.03-2.97 (m, 2H), 2.97-2.54 (m, 5H), 2.41-2.34 (m, 2H), 2.29 (t, J=6.4 Hz, 2H), 1.71-1.65 (m, 1H), 1.60-1.51 (m, 1H), 1.38-1.31 (m, 2H), 1.31-1.24 (m, 2H) ppm. One proton in carboxyl group is not revealed.

### Step 7

A solution of compound 96-9 ((2S)-2-[1-({[(9H-fluoren-9-yl)methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (96-9, 125 mg, 0.081 mmol)), compound 96-10 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-10, 90.97 mg, 0.081 mmol)) and DIPEA (20.89 mg, 0.162 mmol) in anhydrous DMF (1.5 mL) was stirred at room temperature for 5 min, then a solution of HATU (30.79 mg, 0.081 mmol) in anhydrous DMF was added dropwise by syringe over 5 min. After addition, the resulting solution was stirred for another 1 h until all starting amine was consumed (monitored by LCMS). Then the reaction solution was purified directly by reverse phase flash chromatography (0.01% TFA) to yield compound 96-11 ((9H-fluoren-9-yl)methyl N-(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (96-11, 100 mg, 0.038 mmol, 46.62%)) as a white solid, mixed with starting acid. ESI m/z: 883.9 (M/3+H)⁺, 630.0 (linker fragment, (M−717−26−18)/3+H)⁺, purity 60%-66%. Impurity acid ESI m/z: 515.5 (M/3+H)⁺,772.6 (M/2+H)⁺, content 35%-29%.

### Step 8

A solution of compound 96-11 ((9H-fluoren-9-yl)methyl N-(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1- hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (96-11, 100 mg, 0.038 mmol)) in DMF (1.8 mL) was stirred at room temperature and diethyl amine (0.2 mL, 1.941 mmol) was added. The resulting solution was stirred for 1 h to completion. Volatiles were evaporated off under vacuo, and the residue in DMF was purified by reverse phase flash chromatography (0.01% TFA) to give the expected fractions, which were lyophilized by LabConco to yield product 96-12 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-12, 75 mg, 0.031 mmol, 81.32%)) as a white solid. ESI m/z: 809.9 (M/3+H)⁺, 718.6 (fragment, MMAE), 555.9 (linker fragment, (M−717−26−18)/3+H)⁺.

### Step 9

A solution of 4-{2-azatricyclo[10.4.0.0\hat{ }{4,9}]hexadeca-1(16),4(9),5,7,12,14-hexaen-10-yn-2-yl}-4-oxobutanoic acid (DBCO-acid, 710 mg, 2.328 mmol) and HOSu (401.56 mg, 3.492 mmol) in anhydrous DCM (23 mL) was stirred at room temperature for 5 min, then EDCI (669.38 mg, 3.492 mmol) was added. The resulting solution was stirred for another 1.5 h. Then the resulting solution was washed with water (10 mL), the DCM layer was separated and the water layer was extracted with more DCM (20 mL*2). The combined DCM phase was concentrated to dryness and the residue was purified by reverse phase flash chromatography (neutral eluent) to yield compound 96-13 (2,5-dioxopyrrolidin-1-yl 4-{2-azatricyclo[10.4.0.0\hat{ }{4,9}]hexadeca-1(12),4(9),5,7,13,15-hexaen-10-yn-2-yl}-4-oxobutanoate (96-13, 850 mg, 2.114 mmol, 90.83%)) as a white solid. ESI m/z: 403.2 (M+H)⁺, 425.2 (M+Na)⁺.

### Step 10

A solution of compound 96-12 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-12, 65 mg, 0.027 mmol)) and DIPEA (6.91 mg, 0.054 mmol) in anhydrous DMF (2 mL) was stirred at room temperature for 5 min, then a solution of DBCO NHS ester (compound 96-13, 10.78 mg, 0.027 mmol) in anhydrous DMF (2 mL) was added dropwise by syringe. The resulting solution was stirred for another 1 h until LCMS indicated all starting amine was consumed. The resulting solution was then purified directly by Prep-HPLC (10 mM ammonium bicarbonate) to yield PB096 ({4-[(2S)-2-[(2S)-2-[(2S)-2-[1-(4-{2-azatricyclo[10.4.0.0\hat{ }{4,9}]hexadeca-1(12),4(9),5,7,13,15-hexaen-10-yn-2-yl}-4-oxobutanamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB096, 45 mg, 0.017 mmol, 61.90%)) as a white solid. ESI m/z: 905.3 (M/3+H)⁺, 679.2 (M/4+H)⁺, retention time 5.577 min (HPLC). ¹H NMR (400 MHz, DMSO-d6) δ 10.05 (s, 1H), 8.37-8.10 (m, 2H), 8.05-8.03 (m, 1H), 7.93-7.90 (m, 1H), 7.83-7.77 (m, 2H), 7.77-7.67 (m, 2H), 7.63-7.57 (m, 3H), 7.51-7.45 (m, 3H), 7.38-7.26 (m, 9H), 7.20-7.14 (m, 1H), 6.00-5.97 (m, 1H), 5.43-5.36 (m, 3H), 5.11-4.94 (m, 3H), 4.76-4.17 (m, 16H), 4.04-3.92 (m, 2H), 3.79-3.57 (m, 12H), 3.50-3.41 (m, 56H), 3.36-3.17 (m, 19H), 3.12-2.85 (m, 15H), 2.60-2.56 (m, 2H), 2.43-2.36 (m, 3H), 2.30-2.22 (m, 4H), 2.15-1.96 (m, 6H), 1.78-1.21 (m, 20H), 1.05-0.97 (m, 6H), 0.89-0.77 (m, 24H) ppm.

Drug linker PB096 can be used to make a conjugate such as PA096.

### Example 28: Preparation of the Drug-Linker (PB097) Containing a PEG Unit and a Cleavable Linker Attached to Exatecan

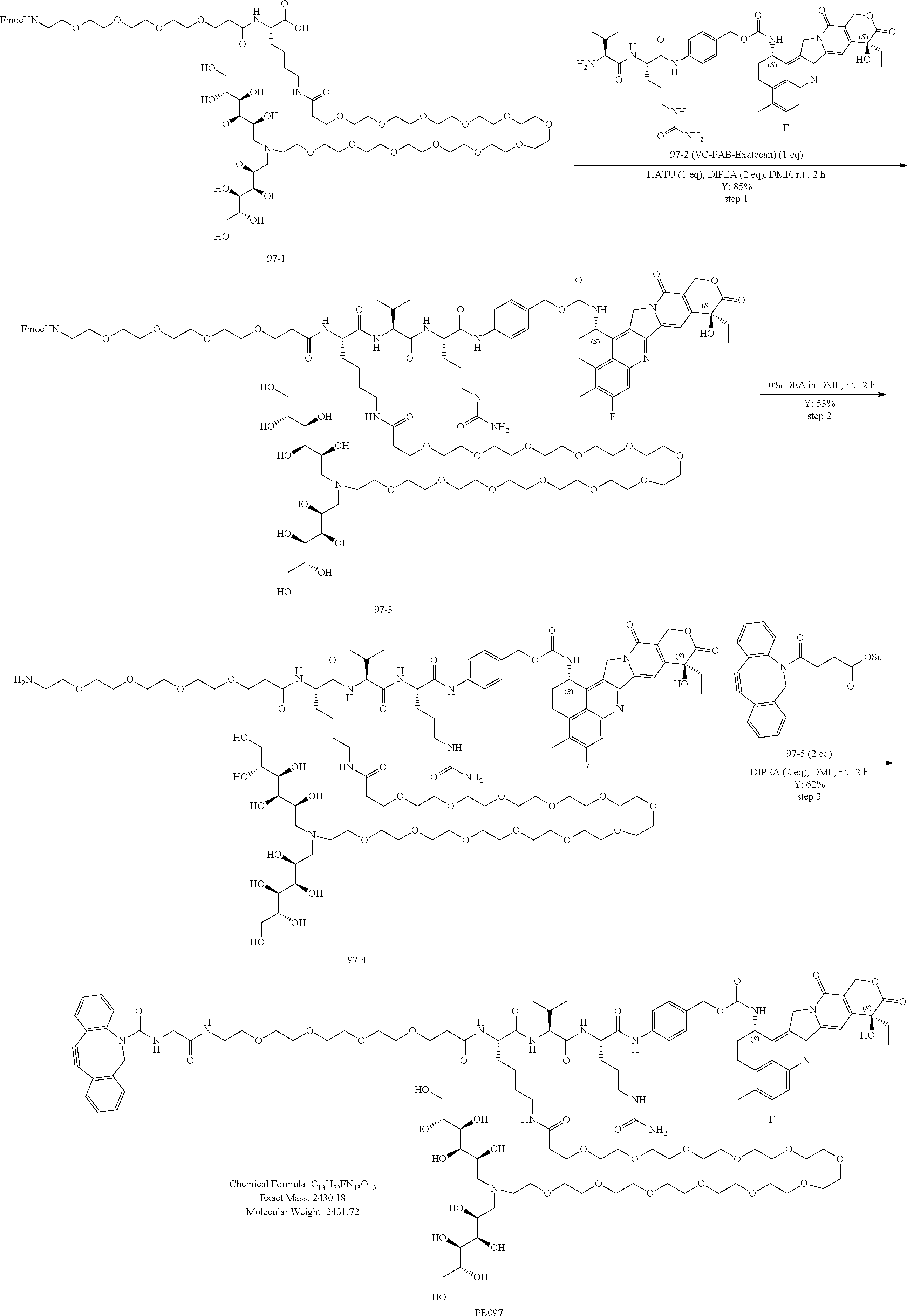

A Drug-Linker containing a PEG unit and a cleavable linker attached to exatecan (PB097) was prepared as follows:

### Step 1

A solution of compound 97-1 ((2S)-2-(1-{[(9H-fluoren-9-ylmethoxy)carbonyl]amino}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (97-1, 200 mg, 0.130 mmol)), compound 97-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamate (97-2, 108.83 mg, 0.129 mmol)) and HATU (49.43 mg, 0.130 mmol), DIPEA (33.59 mg, 0.260 mmol) in DMF (2 mL) was stirred at room temperature for 2 h and LCMS showed that the reaction was completed. The reaction mixture was then purified by reverse phase flash chromatography (0.01% TFA) to get the desired fractions, which were freeze-dried to yield compound 97-3 (9H-fluoren-9-ylmethyl N-(14-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (97-3, 262 mg, 0.111 mmol, 85.16%)) as a pale yellow solid. ESI m/z: 789.7 (M/3+H)⁺.

### Step 2

To a solution of compound 97-3 ((9H-fluoren-9-yl)methyl N-(14-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (97-3, 162 mg, 0.068 mmol)) in DMF (2 mL) was added diethyl amine (0.2 mL, 0.2720 mmol). The mixture was stirred at room temperature for 2 hours. Then the crude mixture was purified by reverse phase flash chromatography (0.01% TFA) to get the desired fractions, which were freeze-dried to yield compound 97-4 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}.0\hat{ }{20,24}]tetracosa-1,6(11),12,14,16,18,20(24)-heptaen-23-yl]carbamate (97-4, 78 mg, 0.036 mmol, 53.49%)) as a white solid. ESI m/z: 715.7 (M/3+H)⁺.

### Step 3

A solution of compound 97-4 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}.0\hat{ }{20,24}]tetracosa-1,6(11),12,14,16,18,20(24)-heptaen-23-yl]carbamate (97-4, 78 mg, 0.036 mmol)), compound 97-5 (2,5-dioxopyrrolidin-1-yl 4-{2-azatricyclo[10.4.0.0^{4,9}]hexadeca-1(12),4(9),5,7,13,15-hexaen-10-yn-2-yl}-4-oxobutanoate (97-5, 29.27 mg, 0.073 mmol) and DIPEA (9.40 mg, 0.073 mmol)) in DMF (2 mL) was stirred at room temperature for 2 h to achieve complete conversion. Then the reaction mixture was purified by Prep-HPLC (10 mM NH₄HCO₃) to get the desired fractions, which were lyophilized by LabConco to yield PB097 ({4-[(2S)-2-[(2S)-2-[(2S)-2-[1-(4-{2-azatricyclo[10.4.0.0\hat{ }{4,9}]hexadeca-1(12),4(9),5,7,13,15-hexaen-10-yn-2-yl}-4-oxobutanamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}.0\hat{ }{20,24}]tetracosa-1,6(11),12,14,16,18,20(24)-heptaen-23-yl]carbamate (PB097, 55 mg, 0.023 mmol, 62.18%)) as a white solid. ESI m/z: 811.5 (M/3+H)⁺. [Note: product was partly cleaved on amide bond and DBCO fragment was released in acidic LCMS, so the DBCO unit was detected with m/z 206 and corresponding counterpart with m/z 749 ((M−205+17)/3+H)⁺ was detected. In the case of basic LCMS, the exatecan lactone ring was partly opened and fragment ion detected with m/z 817 ((M+18)/3+H)⁺]. ¹H NMR (400 MHz, DMSO-d6) δ 10.04 (s, 1H), 8.13 (d, J=8.0 Hz, 1H), 8.09-8.02 (m, 2H), 7.84-7.76 (m, 3H), 7.74-7.66 (m, 2H), 7.62-7.59 (m, 3H), 7.52-7.42 (m, 3H), 7.40-7.27 (m, 6H), 6.54 (s, 1H), 5.99 (t, J=6.4 Hz, 1H), 5.45-5.43 (m, 4H), 5.34-5.23 (m, 3H), 5.08 (s, 2H), 5.02 (d, J=14.0 Hz, 1H), 4.60-4.15 (m, 12H), 3.71-3.67 (m, 1H), 3.64-3.55 (m, 9H), 3.50-3.40 (m, 62H), 3.40-3.39 (m, 4H), 3.32-3.18 (m, 4H), 3.18-2.90 (m, 8H), 2.60-2.51 (m, 1H), 2.42-2.36 (m, 6H), 2.29 (t, J=6.0 Hz, 2H), 2.24-2.09 (m, 4H), 2.03-1.93 (m, 2H), 1.91-1.80 (m, 2H), 1.80-1.52 (m, 5H), 1.49-1.19 (m, 8H), 0.89-0.80 (m, 9H) ppm.

Drug linker PB097 can be used to make a conjugate such as PA097.

### Example 29: Preparation of the Drug-Linker (PB098) Containing a PEG Unit and a Cleavable Linker Attached to MMAE

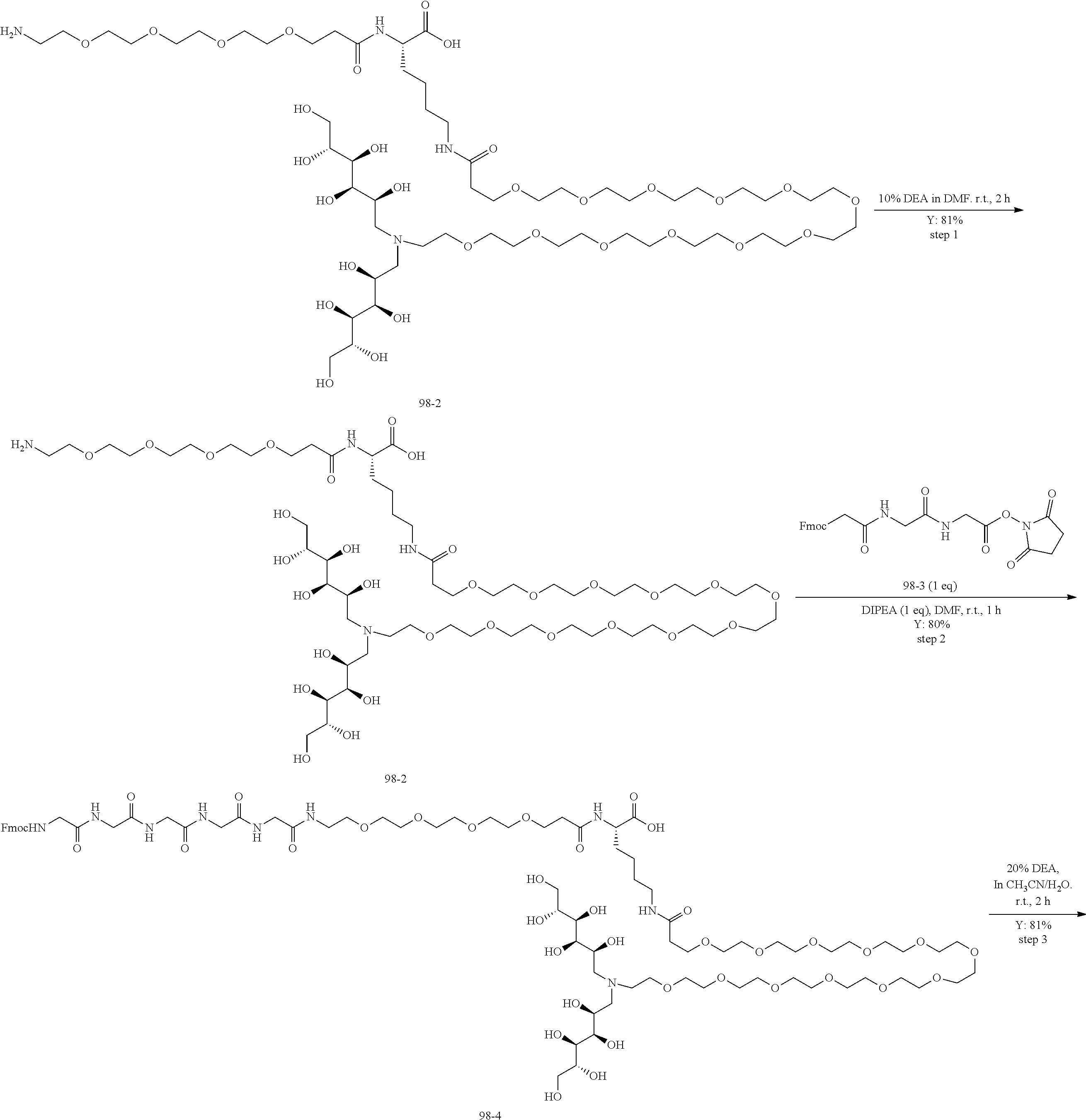

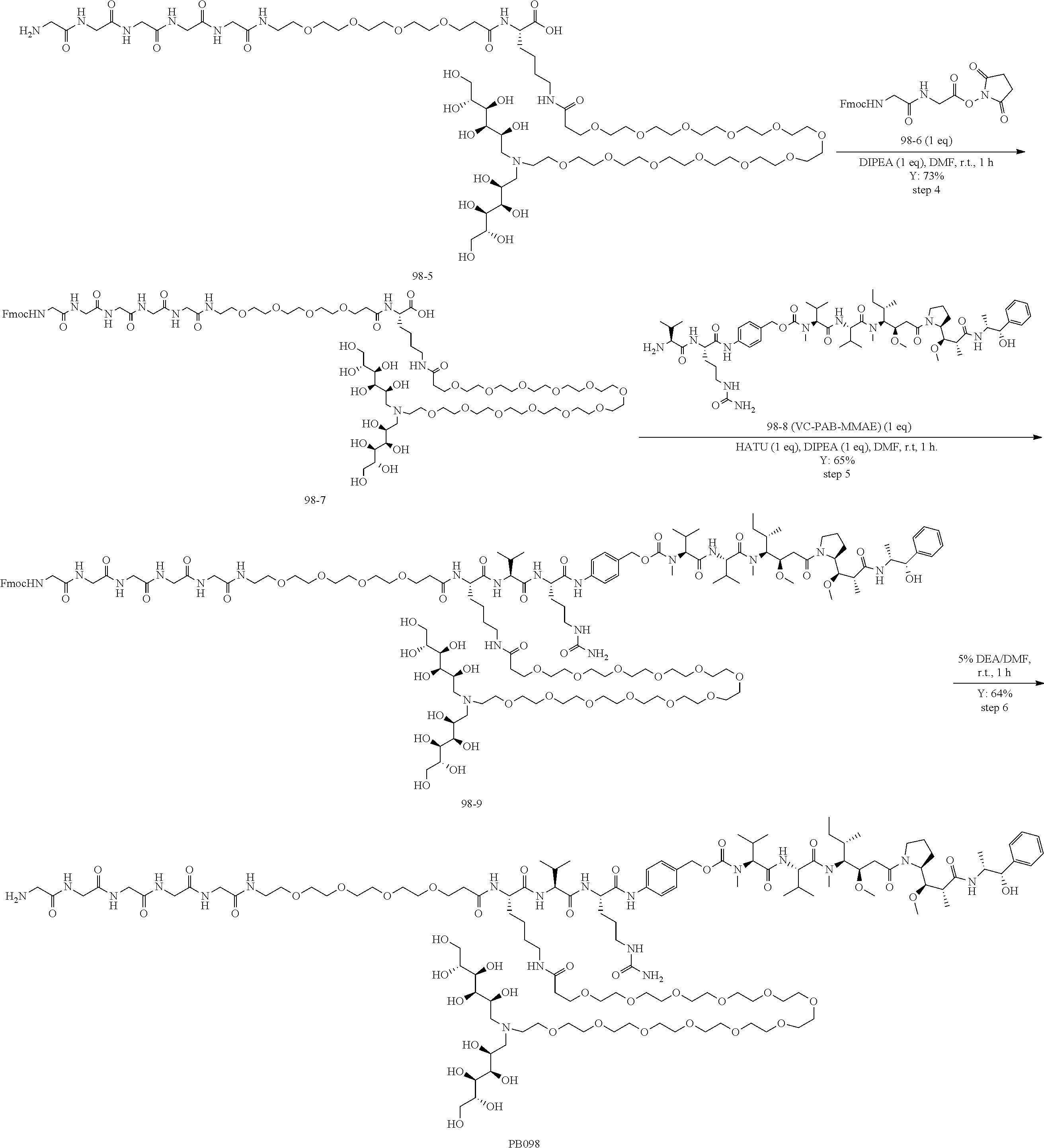

A Drug-Linker containing a PEG unit and a cleavable linker attached to MMAE (PB098) was prepared as follows:

### Step 1

To a solution of compound 98-1 ((2S)-2-[1-({[(9H-fluoren-9-yl) methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-1, 3.0 g, 1.943 mmol)) in DMF (10 mL) was added diethyl amine (1.1 mL, 14.032 mmol). The mixture was stirred at room temperature for 2 h to achieve complete deprotection. Then the resulting solution was purified directly by reverse phase flash chromatography (0.01% TFA) to afford the product 98-2 ((2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-2, 2.1 g, 1.589 mmol, 81.71%)) as white solid. ESI m/z: 661.6 (M/2+H)⁺.

### Step 2

To a solution of compound 98-2 ((2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-2, 2.1 g, 1.589 mmol)) in DMF (10 mL) was added compound 98-3 (pentafluorophenyl 2-{2-[2-({[(9H-fluoren-9-yl)methoxy]carbonyl}amino)acetamido]acetamido}acetate (98-3, 0.92 g, 1.589 mmol)) and DIPEA (0.21 g, 1.589 mmol). The mixture was stirred at room temperature for 1 h. The resulting solution was purified by reverse phase flash chromatography (0.01% TFA) to afford the product 98-4 ((2S)-2-[1-(2-{2-[2-({[(9H-fluoren-9-yl)methoxy]carbonyl}amino)acetamido]acetamido}acetamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-4, 2.2 g, 1.283 mmol, 80.59%)) as a white solid. ESI m/z: 858.2 (M/2+H)⁺.

### Step 3

To the solution of compound 98-4 ((2S)-2-[1-(2-{2-[2-({[(9H-fluoren-9-yl)methoxy]carbonyl}amino)acetamido]acetamido}acetamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-4, 2.2 g, 1.283 mmol)) in MeCN (10 mL) and H₂O (6 mL) was added diethyl amine (4 mL, 2.840 mmol). The mixture was stirred at room temperature for 2 h to completion. The resulting solution was concentrated to dryness and purified by reverse phase flash chromatography (0.01% TFA) to afford the product 98-5 ((2S)-2-(1-{2-[2-(2-aminoacetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-5, 1.55 g, 1.038 mmol, 81.15%)) as white gel. ESI m/z: 747.1 (M/2+H)⁺, 498.5 (M/3+H)⁺.

### Step 4

To a solution of compound 98-5 ((2S)-2-(1-{2-[2-(2-aminoacetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-5, 1.55 g, 1.038 mmol)) in DMF (10 mL) was added compound 98-6 (2,5-dioxopyrrolidin-1-yl 2-[2-({[(9H-fluoren-9-yl)methoxy]carbonyl}amino)acetamido]acetate (98-6, 0.47 g, 1.038 mmol)) and DIPEA (0.13 g, 1.038 mmol). The mixture was stirred at room temperature for 1 h. The resulting solution was purified by reverse phase flash chromatography (0.01% TFA) to afford the product 98-7 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-7, 1.4 g, 0.765 mmol, 73.68%)) as a white solid. ESI m/z: 915.2 (M/2+H)⁺, 610.7 (M/3+H)⁺. ¹H NMR (400 MHz, DMSO-d6) δ 8.22-8.15 (m, 4H), 8.11-8.08 (m, 2H), 7.91-7.86 (m, 3H), 7.82 (t, J=5.6 Hz, 1H), 7.72 (d, J=7.6 Hz, 2H), 7.59 (t, J=6.0 Hz, 1H), 7.44-7.40 (m, 2H), 67.36-7.31 (m, 2H), 4.55-4.12 (m, 10H), 3.80-3.72 (m, 8H), 3.69-3.66 (m, 4H), 3.62-3.56 (m, 10H), 3.53-3.47 (m, 60H), 3.42-3.38 (m, 5H), 3.32-3.19 (m, 2H), 3.03-2.92 (m, 8H), 2.50-2.36 (m, 2H), 2.29 (t, J=6.4 Hz, 2H), 1.72-1.63 (m, 1H), 1.59-1.50 (m, 1H), 11.41-1.35 (m, 2H), 1.31-1.16 (m, 5H) ppm.

### Step 5

To the solution of compound 98-7 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-7, 200 mg, 0.109 mmol)) and compound 98-8 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (98-8, 122.85 mg, 0.109 mmol)) in dry DMF (5 mL) was added HATU (41.58 mg, 0.109 mmol) and DIPEA (14.13 mg, 0.109 mmol) sequentially. After addition, the mixture was stirred at room temperature for 1 h until all starting amine was consumed (monitored by LCMS). Then the resulting solution was purified by reverse phase flash chromatography (0.01% TFA) to get the desired fractions, which were lyophilized to yield the product 98-9 ((9H-fluoren-9-yl)methyl N-[({[({[({[(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)methyl]carba mate (98-9, 210 mg, 0.072 mmol, 65.65%)) as white solid. ESI m/z: 979.0 (M/3+H)⁺.

### Step 6

To a solution of compound 98-9 ((9H-fluoren-9-yl)methyl N-[({[({[({[(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)methyl]carba mate (98-9, 210 mg, 0.072 mmol)) in anhydrous DMF (1.9 mL) was added diethyl amine (0.1 mL, 0.971 mmol). The solution was stirred at room temperature for 1 h to completion. The resulting solution was purified directly by Prep-HPLC (0.01% TFA) to afford a TFA salt of product PB098 ({4-[(2S)-2-[(2S)-2-[(2S)-2-{1-[2-(2-{2-[2-(2-aminoacetamido)acetamido]acetamido}acetamido)acetamido]-3,6,9,12-tetraoxapentadecan-15-amido}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB098, 125 mg, 0.046 mmol, 64.40%)) as a white solid. ESI m/z: 905.0 (M/3+H)⁺, retention time 5.432 min (HPLC). ¹H NMR (400 MHz, DMSO-d6) δ 10.05 (s, 1H), 8.64 (t, J=5.6 Hz, 1H), 8.36-8.01 (m, 9H), 7.93-7.91 (m, 1.5H), 7.84-7.81 (m, 1H), 7.73 (d, J=8.4 Hz, 0.5H), 7.58 (d, J=7.6 Hz, 2H), 7.35-7.24 (m, 6H), 7.20-7.14 (m, 1H), 6.05-5.96 (m, 1H), 5.44 (brs, 4H), 5.13-4.94 (m, 2H), 4.82-4.38 (m, 10H), 4.30-4.17 (m, 4H), 4.04-3.93 (m, 4H), 3.86 (d, J=5.6 Hz, 2H), 3.78-3.74 (m, 7H), 3.69-3.67 (m, 5H), 3.62-3.55 (m, 14H), 3.55-3.39 (m, 56H), 3.39-3.30 (m, 5H), 3.25-3.04 (m, 11H), 3.05-2.83 (m, 10H), 2.41-2.36 (m, 3H), 2.33-2.27 (m, 3H), 2.16-1.93 (m, 4H), 1.81-1.21 (m, 18H), 1.05-0.97 (m, 6H), 0.89-0.73 (m, 27H) ppm. One proton of carboxyl group on TFA was revealed.

Drug linker PB098 can be used to make a conjugate such as PA098.

### Example 30: Preparation of the Drug-Linker (PB099) Containing a PEG Unit and a Cleavable Linker Attached to Exatecan

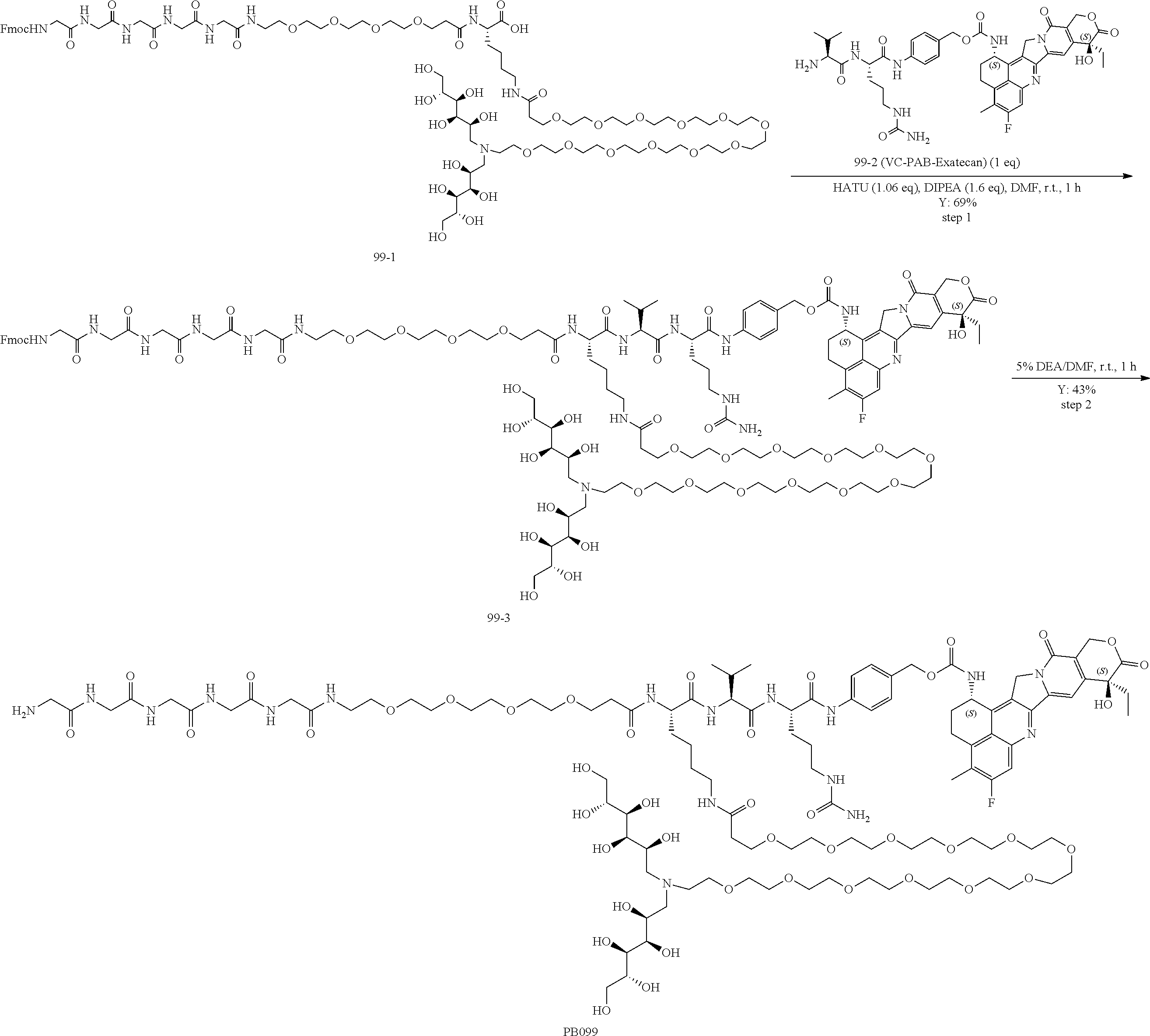

A Drug-Linker containing a PEG unit and a cleavable linker attached to exatecan (PB099) was prepared as follows:

### Step 1

To a solution of compound 99-1 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (99-1, 190 mg, 0.104 mmol)) in DMF (4 mL) was added compound 99-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (99-2, 88 mg, 0.105 mmol)), HATU (41.47 mg, 0.109 mmol) and DIPEA (0.207 mL, 0.156 mmol). The mixture was stirred at room temperature for 1 h to complete (monitored by LCMS). Then resulting solution was purified by reverse phase flash chromatography (0.01% TFA) to afford the product 99-3 ((9H-fluoren-9-yl)methyl N-[({[({[({[(14-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)methyl]carba mate (99-3, 190 mg, 0.072 mmol, 68.97%)) as a yellow solid. ESI m/z: 885.0 (M/3+H)⁺,663.9 (M/4+H)⁺.

### Step 2

To the solution of compound 99-3 ((9H-fluoren-9-yl)methyl N-[({[({[({[(14-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0{6,111}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)methyl]carba mate (99-3, 102 mg, 0.038 mmol)) in DMF (1.9 mL) was added diethyl amine (0.1 mL, 0.971 mmol). The mixture was stirred at room temperature for 1 h to completion. The resulting solution was purified by reverse phase separation (0.01% TFA) to afford a TFA salt of product PB099 ({4-[(2S)-2-[(2S)-2-[(2S)-2-{1-[2-(2-{2-[2-(2-aminoacetamido)acetamido]acetamido}acetamido)acetamido]-3,6,9,12-tetraoxapentadecan-15-amido}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB099, 40 mg, 0.016 mmol, 42.80%)) as a yellow solid. ESI m/z: 810.8 (M/3+H)⁺, 608.3 (M/4+H)⁺, retention time 5.077 min (HPLC). ¹H NMR (400 MHz, DMSO-d6) δ 10.05 (s, 1H), 8.64 (t, J=5.6 Hz, 1H), 8.32 (t, J=5.6 Hz, 1H), 8.21-8.01 (m, 9H), 7.92 (t, J=5.6 Hz, 1H), 7.85-7.72 (m, 3H), 7.60 (d, J=8.4 Hz, 2H), 7.36 (d, J=8.4 Hz, 2H), 7.31 (s, 1H), 6.55 (s, 1H), 6.02 (t, J=5.6 Hz, 1H), 5.46 (brs, 6H), 5.34-5.23 (m, 3H), 5.08 (s, 2H), 4.83 (brs, 2H), 4.56 (brs, 4H), 4.41-4.25 (m, 3H), 4.21-4.17 (m, 1H), 3.99 (brs, 2H), 3.86 (d, J=5.2 Hz, 2H), 3.78-3.73 (m, 6H), 3.69-3.67 (m, 4H), 3.62-3.54 (m, 14H), 3.52-3.47 (m, 60H), 3.29-3.20 (m, 6H), 3.14-2.90 (m, 6H), 2.41-2.37 (m, 5H), 2.29 (t, J=6.4 Hz, 2H), 2.24-2.13 (m, 2H), 1.99-1.81 (m, 3H), 1.70-1.58 (m, 3H), 1.50-1.20 (m, 8H), 0.90-0.81 (m, 9H) ppm. Two proton of carboxyl group on TFA were revealed. Drug linker PB099 can be used to make a conjugate such as PA099.

### Example 31: Preparation of the Drug-Linker (PB100) Containing a PEG Unit and a Cleavable Linker Attached to Exatecan

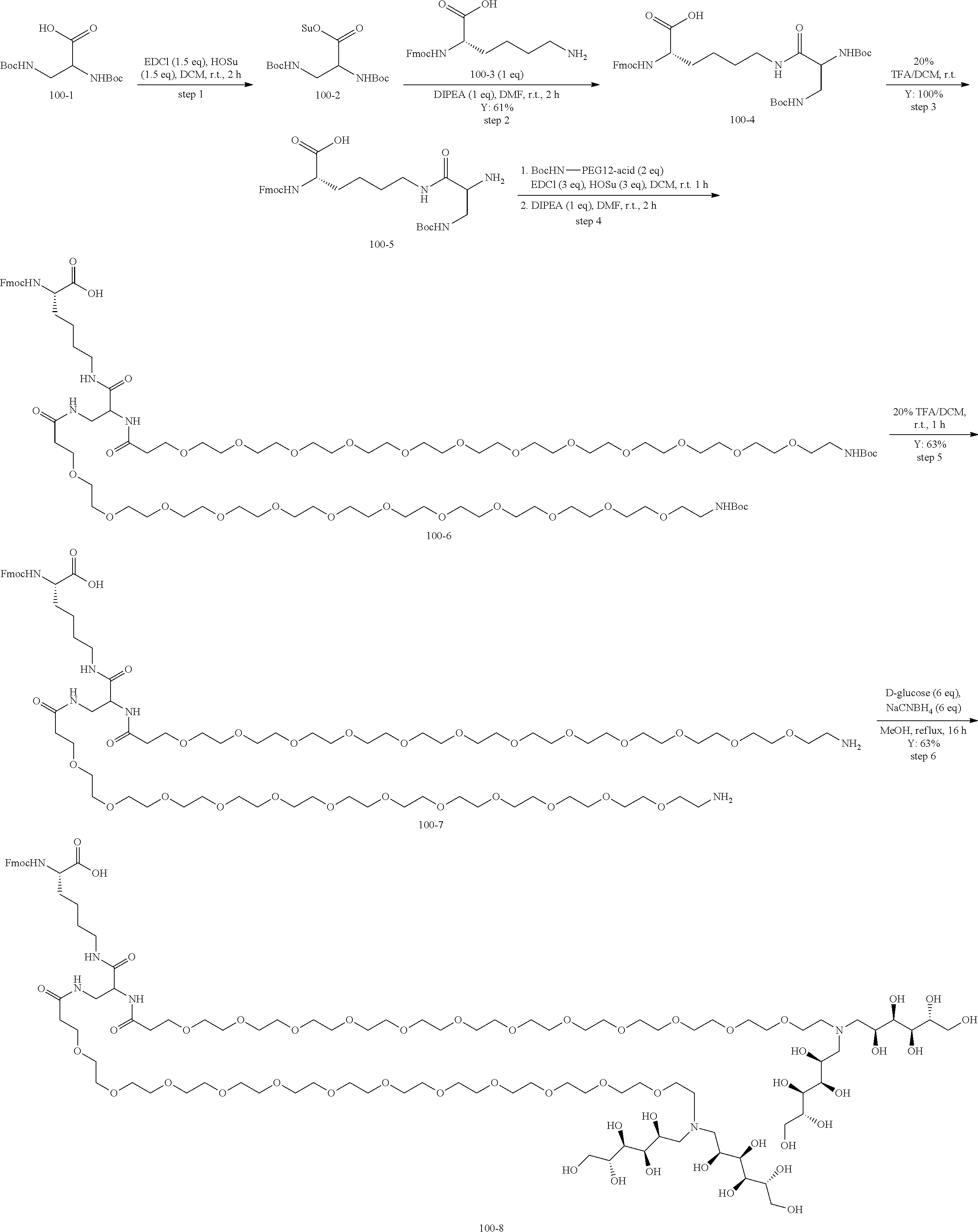

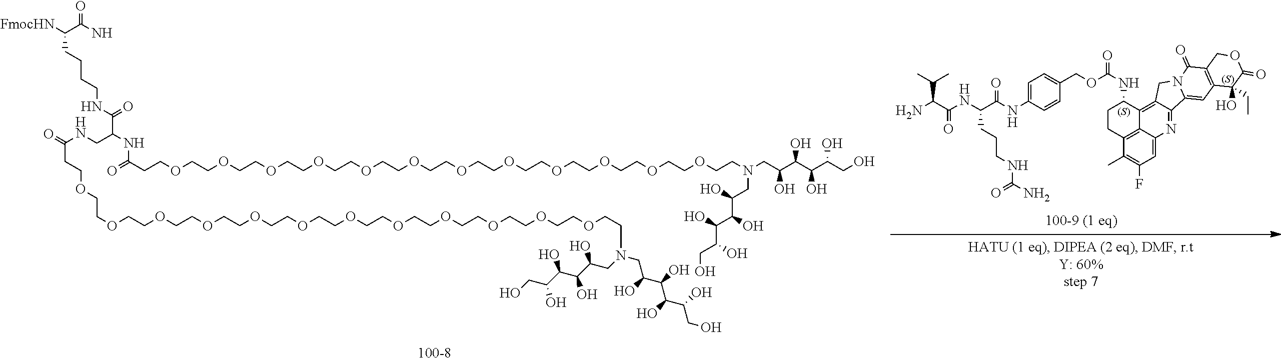

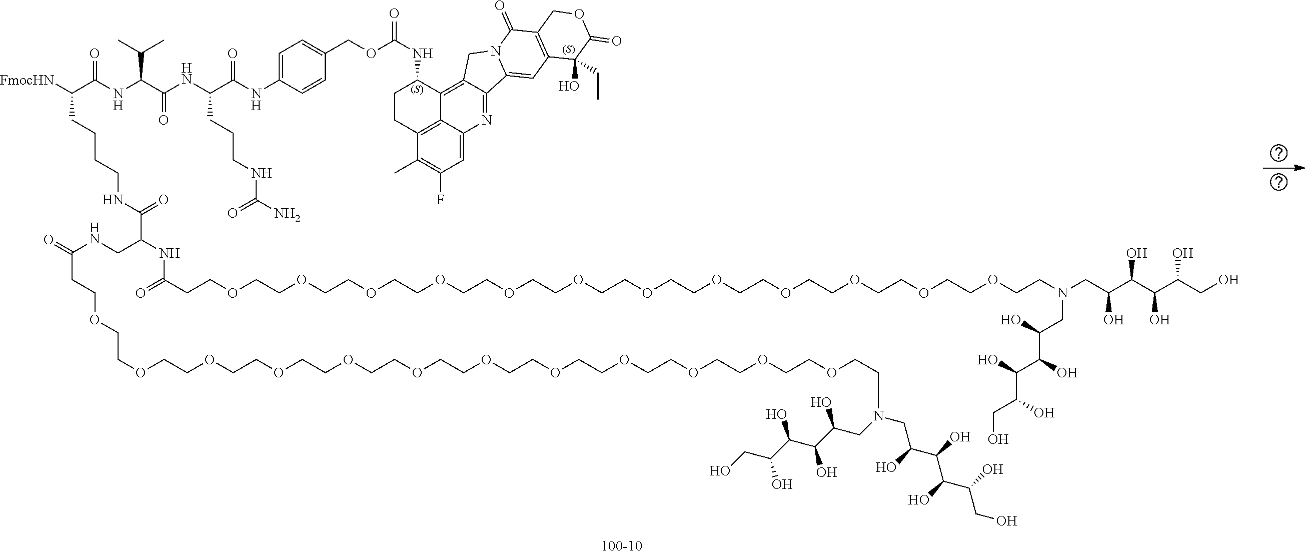

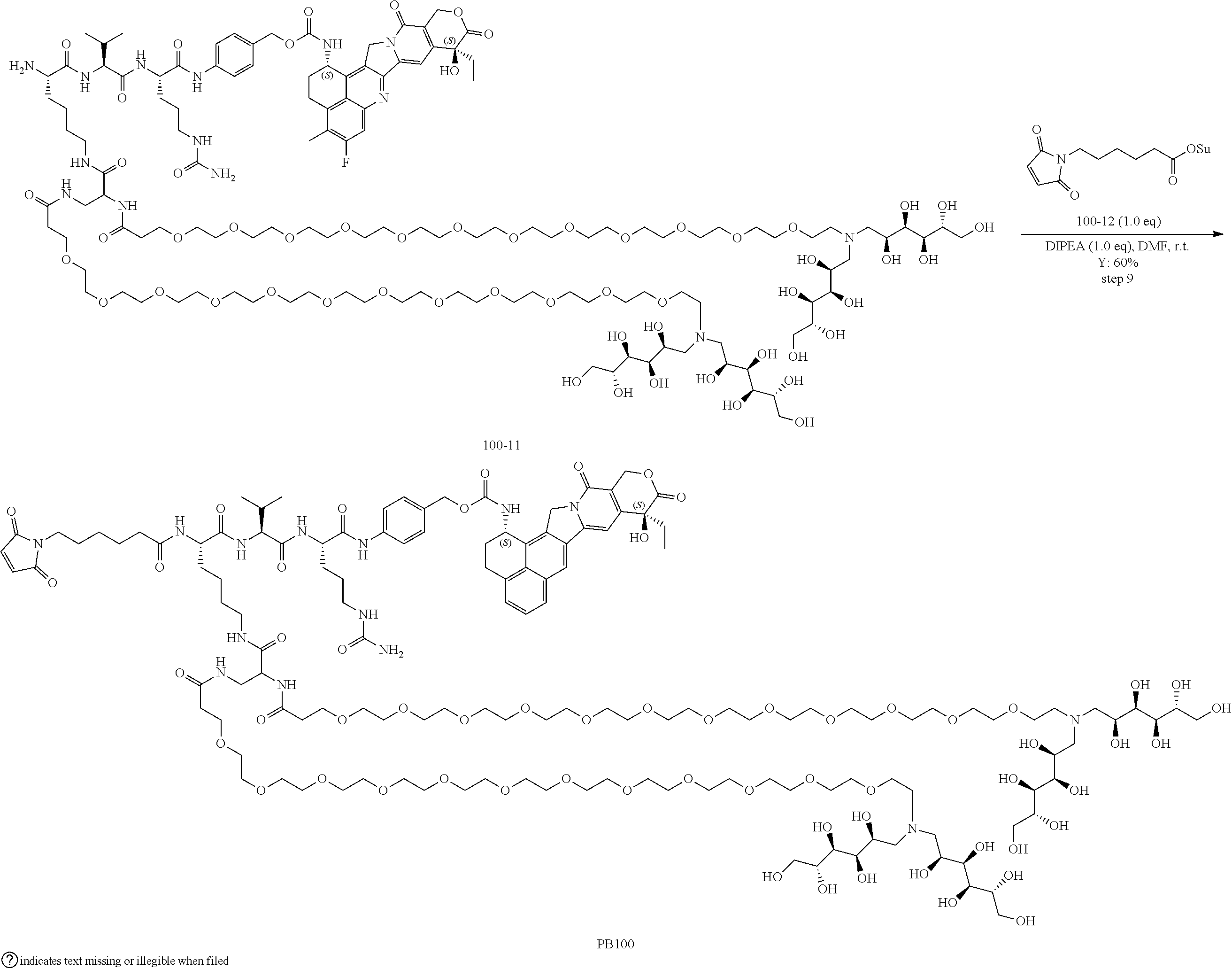

A Drug-Linker containing a PEG unit and a cleavable linker attached to exatecan (PB100 or LD100) was prepared as follows:

### Step 1

A solution of compound 100-1 (2,3-bis({[(tert-butoxy)carbonyl]amino})propanoic acid (100-1, 1.52 g, 5.000 mmol)) and HOSu (0.86 g, 7.500 mmol) in anhydrous DCM (20 mL) was stirred at room temperature, then EDCI (1.44 g, 7.500 mmol) was added portionwise over 5 min. The resulting solution was stirred for another 1 h until the starting acid was consumed (monitored by LCMS). Then the reaction solution was diluted with more DCM (20 mL) and washed with water (40 mL), the organic layer was separated and the water phase was extracted with more DCM (2*40 mL). The collected DCM layers were dried over sodium sulfate, filtered and concentrated the filtrate to give crude compound 100-2 (2,5-dioxopyrrolidin-1-yl 2,3-bis((tert-butoxycarbonyl)amino)propanoate (100-2, 2.0 g, 4.988 mmol, 99.75%)) as a white foam, which turned into colorless oil after standing for minutes. The compound was used as such in the next step. ESI m/z: 424.2 (M+Na)⁺.

### Step 2

A solution of compound 100-3 ((2S)-6-amino-2-({[(9H-fluoren-9-yl)methoxy]carbonyl}amino)hexanoic acid (100-3, 1.84 g, 4.988 mmol)) and DIPEA (0.64 g, 4.988 mmol) in dry DMF (10 mL) was stirred at room temperature for 10 min, then a solution of compound 100-2 (2,5-dioxopyrrolidin-1-yl 2,3-bis({[(tert-butoxy)carbonyl]amino})propanoate (100-2, 2.0 g, 4.988 mmol)) in anhydrous DMF (10 mL) was added slowly over 5 min. After addition, the resulting suspension was stirred for 2 h until all starting materials were consumed. The resulting solution was purified directly by reverse phase flash chromatography (0.01% TFA) to yield compound 100-4 ((2S)-6-[2,3-bis({[(tert-butoxy)carbonyl]amino})propanamido]-2-({[(9H-fluoren-9-yl)methoxy]carbonyl}amino)hexanoic acid (100-4, 2.0 g, 3.055 mmol, 61.16%)) as a white solid. ESI m/z: 555.3 (M−100+H)⁺,678.4 (M+Na)⁺. ¹H NMR (400 MHz, DMSO-d6) δ 12.55 (s, 1H), 7.90 (d, J=7.6 Hz, 2H), 7.84 (t, J=4.8 Hz, 1H), 7.73 (d, J=7.2 Hz, 2H), 7.62 (d, J=8.0 Hz, 1H), 7.44-7.40 (m, 2H), 7.36-7.31 (m, 2H), 6.73 (m, 1H), 6.61 (d, J=8.0 Hz, 1H), 4.29-4.21 (m, 3H), 3.98-3.87 (m, 2H), 3.21-3.16 (m, 2H), 3.11-2.94 (m, 2H), 1.74-1.55 (m, 2H), 1.39-1.26 (m, 22H) ppm.

### Step 3

A solution of compound 100-4 ((2S)-6-[2,3-bis({[(tert-butoxy)carbonyl]amino})propanamido]-2-({[(9H-fluoren-9-yl)methoxy]carbonyl}amino)hexanoic acid (100-4, 1.0 g, 1.527 mmol)) in DCM (8 mL) was stirred at room temperature, then TFA (2 mL, 26.925 mmol) was added slowly. The solution was stirred for another 1 h, then the solution was evaporated to dryness. The residue was dissolved with DCM (20 mL) again and concentrated. The process was repeated twice to yield crude product 100-5 ((2S)-6-(2,3-diaminopropanamido)-2-({[(9H-fluoren-9-yl)methoxy]carbonyl}amino)hexanoic acid (100-5, 1.0 g, 2.200 mmol, 144.05%)) as pale yellow oil, used as such in the next step. ESI m/z: 455.3 (M+H)⁺.

### Step 4

A solution of 1-{[(tert-butoxy)carbonyl]amino}-3,6,9,12,15,18,21,24,27,30,33,36-dodecaoxanonatriacontan-39-oic acid (2.2 g, 3.065 mmol) and HOSu (0.53 g, 4.597 mmol) in anhydrous DCM (10 mL) was stirred at room temperature for 5 min, then EDCI (0.88 g, 4.597 mmol) was added. The resulting solution was stirred for another 1 h until all acid was converted into activated ester. Then the solution was diluted with more DCM (20 mL) and washed with water (20 mL), the organic layer was collected and the water layer was extracted with more DCM (20 mL*2). The DCM layer was combined and dried over sodium sulfate, filtered and concentrated the filtrate to give crude active ester as colorless oil. The activated ester was dissolved in anhydrous DMF (5 mL), and added to a solution of compound 100-5 ((2S)-6-(2,3-diaminopropanamido)-2-({[(9H-fluoren-9-yl)methoxy]carbonyl}amino)hexanoic acid (100-5, 0.63 g, 1.390 mmol)) and DIPEA (0.36 g, 2.780 mmol) in anhydrous DMF (5 mL) slowly. After addition, the resulting pale yellow solution was stirred at room temperature for 2 h to completion. Then the reaction solution was purified by reverse phase flash chromatography (0.01% TFA) to yield compound 100-6 as a colorless oil ((2S)-6-[2,3-bis(1-{[(tert-butoxy)carbonyl]amino}-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-6)). ESI m/z: 551.3 ((M−100)/3+H)⁺, 827.7 (M−100)/2+H)⁺.

### Step 5

A solution of compound 100-6 ((2S)-6-[2,3-bis(1-{[(tert-butoxy)carbonyl]amino}-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-6, 1.8 g, 0.971 mmol)) in DCM (8 mL) was stirred at room temperature, then TFA (2 mL, 26.925 mmol) was added. The resulting solution was stirred for 1 h. Then the solution was concentrated to dryness and the residue was purified by reverse phase flash chromatography (0.01% TFA) to give the expected fractions, which were lyophilized to yield product 100-7 ((2S)-6-[2,3-bis(1-amino-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-7, 1.0 g, 0.605 mmol, 62.28%)) as colorless oil. ESI m/z: 552.3 (M/3+H)⁺, 828.1 (M/2+H)⁺. ¹H NMR (400 MHz, DMSO-d6) δ 7.95-7.89 (m, 3H), 7.85-7.72 (m, 9H), 7.63 (d, J=8.0 Hz, 1H), 7.45-7.41 (m, 2H), 7.36-7.31 (m, 2H), 4.29-4.20 (m, 4H), 3.94-3.88 (m, 1H), 3.68-3.66 (m, 1H), 3.61-3.57 (m, 18H), 3.57-3.47 (m, 76H), 3.34-3.29 (m, 2H), 3.26-3.21 (m, 1H), 3.05-2.97 (m, 6H), 2.39 (t, J=6.8 Hz, 2H), 2.31 (t, J=6.8 Hz, 2H), 1.74-1.55 (m, 2H), 1.44-1.25 (m 4H) ppm.

### Step 6

A suspension of compound 100-7 ((2S)-6-[2,3-bis(1-amino-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-7, 850 mg, 0.514 mmol)) and D-glucose (555.03 mg, 3.084 mmol) in methanol (20 mL) was heated to 50° C. under nitrogen atmosphere and then sodium cyanoborohydride (193.77 mg, 3.084 mmol) was added. The resulting mixture was heated overnight (18 h) and completed. Then the solution was concentrated to dryness, the residue was dissolved in DMF and purified by reverse phase flash chromatography (0.01% TFA) to yield compound 100-8 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (100-8, 640 mg, 0.173 mmol, 53.9%)) as colorless oil. ESI m/z=771.0 (M/3+H)⁺. ¹H NMR (400 MHz, DMSO-d6) δ 12.47 (s, 1H), 8.16 (brs, 2H), 7.95-7.80 (m, 5H), 7.73 (d, J=7.6 Hz, 2H), 7.63 (d, J=8.0 Hz, 1H), 7.45-7.40 (m, 2H), 7.35-7.31 (m, 2H), 5.48-5.41 (m, 4H), 4.85-4.76 (m, 4H), 4.64-4.41 (m, 12H), 4.31-4.20 (m, 4H), 4.04-3.95 (m, 4H), 3.95-3.87 (m, 1H), 3.79-3.76 (m, 5H), 3.72-3.61 (m, 5H), 3.59-3.55 (m, 20H), 3.55-3.40 (m, 98H), 3.29-3.21 (m, 2H), 3.11-2.97 (m, 2H), 2.38 (t, J=6.4 Hz, 2H), 2.31 (t, J=6.4 Hz, 2H), 1.74-1.56 (m, 2H), 1.43-1.26 (m, 4H) ppm. Two protons of carboxyl group from TFA were also revealed.

### Step 7

A solution of compound 100-8 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (100-8, 150 mg, 0.065 mmol)), compound 100-9 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (100-9, 54.59 mg, 0.065 mmol)) and DIPEA (16.75 mg, 0.130 mmol) in anhydrous DMF (1.5 mL) was stirred at room temperature for 5 min to allow starting materials dissolve thoroughly in the solvent, then a solution of HATU (24.68 mg, 0.065 mmol) in anhydrous DMF (0.5 mL) was added. The resulting solution was stirred for another 2 h until LCMS indicated complete reaction. The completed reaction solution was purified directly by reverse phase flash chromatography (0.01% TFA) to yield compound 100-10 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1S)-1-{[(1S)-4-(carbamoylamino)-1-({4-[(({[(1S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0{6,111}.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (100-10, 103 mg, 0.033 mmol, 50.63%)) as a white solid. ESI m/z: 784.2 (M/4+H)⁺.

### Step 8

The solution of compound 100-10 (4-((2S,5S,8S,59S,60R,61R,62R)-8-((((9H-fluoren-9-yl)methoxy)carbonyl)amino)-59,60,61,62,63-pentahydroxy-5-isopropyl-4,7,14,18-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-57-((2S,3R,4R,5R)-2,3,4,5,6-pentahydroxyhexyl)-2-(3-ureidopropyl)-21,24,27,30,33,36,39,42,45,48,51,54-dodecaoxa-3,6,13,17,57-pentaazatrihexacontanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (100-10, 100 mg, 0.032 mmol)) in DMF (1.8 mL) was stirred at room temperature and diethyl amine (0.2 mL, 1.941 mmol) was added. The reaction solution was stirred for 1 h and completed. Most of diethyl amine was evaporated with a rotary evaporator, then the residue was purified by reverse phase flash chromatography (0.01% TFA) to yield the expected product 100-11 (4-((2S,5S,8S,59S,60R,61R,62R)-8-amino-59,60,61,62,63-pentahydroxy-5-isopropyl-4,7,14,18-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-57-((2S,3R,4R,5R)-2,3,4,5,6-pentahydroxyhexyl)-2-(3-ureidopropyl)-21,24,27,30,33,36,39,42,45,48,51,54-dodecaoxa-3,6,13,17,57-pentaazatrihexacontanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (100-11, 50 mg, 0.017 mmol, 53.82%)) as a pale yellow solid. ESI m/z: 971.5 (M/3+H)⁺, 728.9 (M/4+H)⁺.

### Step 9

A solution of compound 100-11 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (100-11, 50 mg, 0.017 mmol)) and DIPEA (3.32 mg, 0.026 mmol) in anhydrous DMF (1.5 mL) was stirred at room temperature for 5 min, then a solution of compound 100-12 (2,5-dioxopyrrolidin-1-yl 6-(2,5-dioxo-2,5-dihydro-1H-pyrrol-1-yl)hexanoate (100-12, 6.35 mg, 0.021 mmol)) in anhydrous DMF (0.5 mL) was added dropwise by syringe over 5 min. The resulting solution was stirred for another 4 h until LCMS indicated all starting amine was consumed. Then the reaction solution was purified directly by Prep-HPLC (0.01% TFA) to yield PB100 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB100, 35 mg, 0.011 mmol, 65.64%)) as a white solid. ESI m/z: 621.9 (M/5+H)⁺, 777.0 (M/4+H)⁺, 1035.7 (M/3+H)⁺. Retention time 5.642 min (HPLC).

¹H NMR (400 MHz, DMSO-d6) δ 10.04 (s, 1H), 8.15-8.13 (m, 3H), 8.07 (d, J=9.2 Hz, 1H), 7.99-7.91 (m, 2H), 7.86-7.77 (m, 3H), 7.64 (d, J=8.8 Hz, 1H), 7.60 (d, J=8.4 Hz, 2H), 7.36 (d, J=8.4 Hz, 2H), 7.32 (s, 1H), 7.00 (s, 2H), 6.54 (s, 1H), 6.01 (t, J=5.6 Hz, 1H), 5.49-5.39 (m, 7H), 5.39-5.24 (m, 3H), 5.08 (s, 2H), 4.83-4.79 (m, 4H), 4.65-4.46 (m, 11H), 4.40-4.35 (m, 1H), 4.30-4.18 (m, 3H), 4.03-3.94 (m, 4H), 3.81-3.74 (m, 4H), 3.74-3.61 (m, 4H), 3.61-3.52 (m, 16H), 3.52-3.40 (m, 98H), 3.29-3.13 (m, 8H), 3.13-2.90 (m, 7H), 2.40-2.36 (m, 5H), 2.30 (t, J=6.4 Hz, 2H), 2.24-2.08 (m, 4H), 2.00-1.71 (m, 4H), 1.71-1.51 (m, 3H), 1.51-1.14 (m, 15H), 0.90-0.80 (m, 9H) ppm. Two protons signal of carboxyl group in TFA are revealed.

Drug linker PB100 can be used to make a conjugate such as PA100.

### Example 32: Preparation of the Drug-Linker (PB101) Containing a PEG Unit and a Cleavable Linker Attached to MMAE

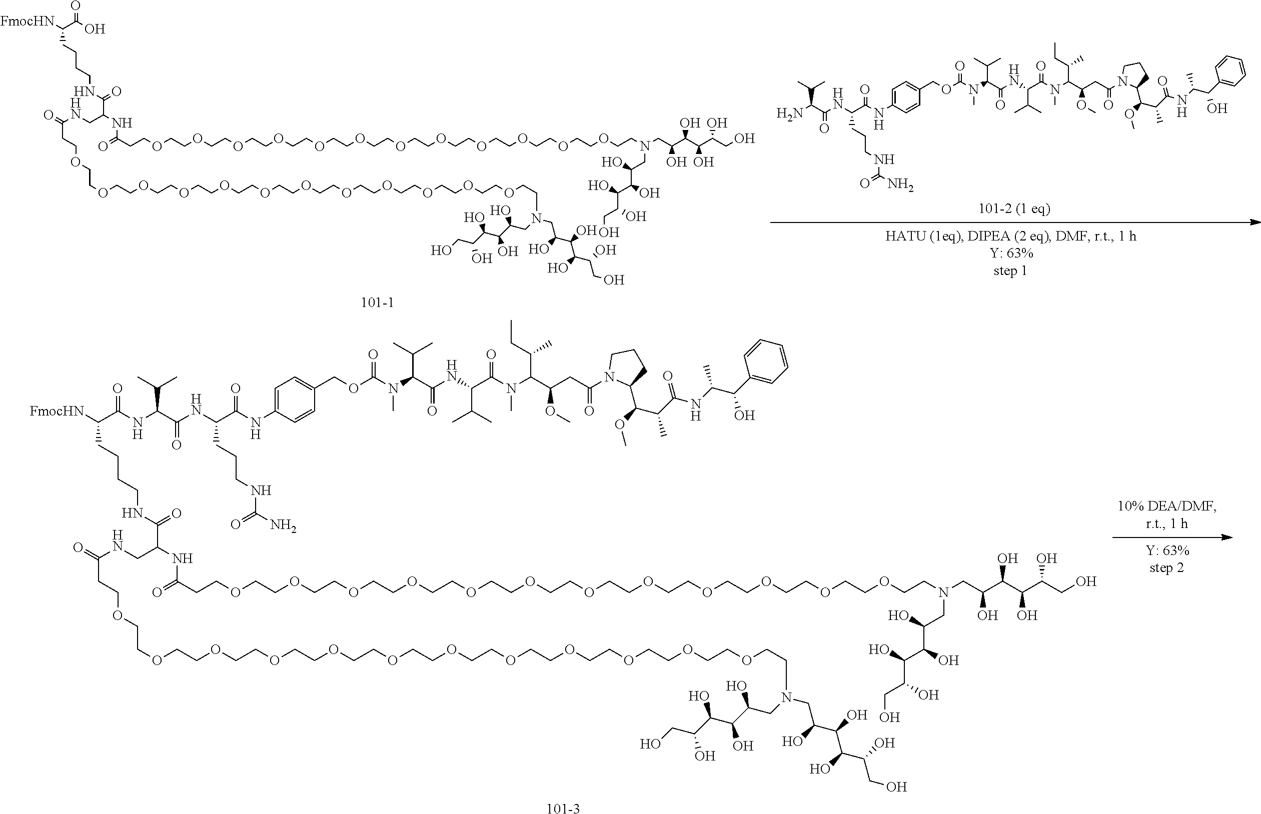

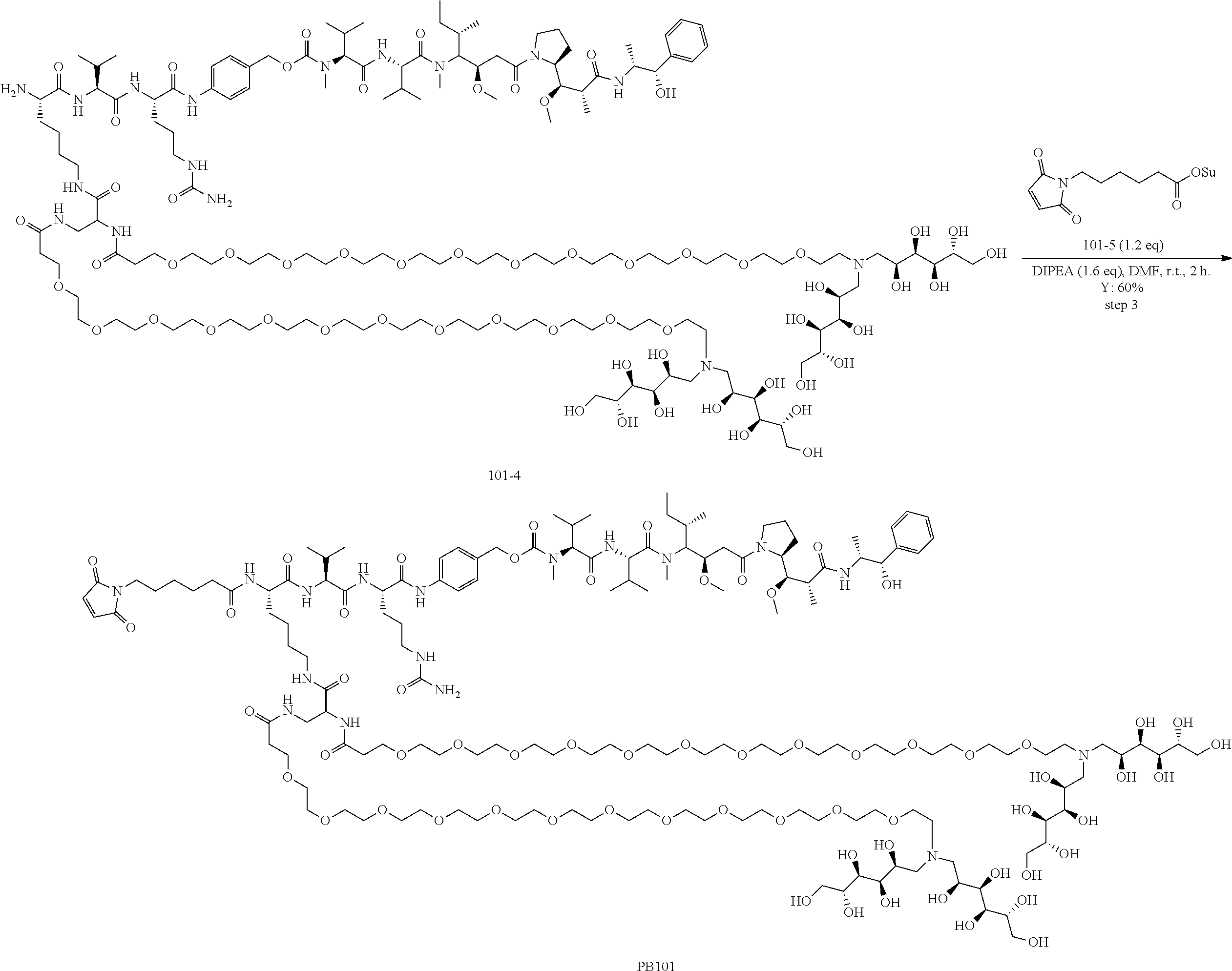

A Drug-Linker containing a PEG unit and a cleavable linker attached to MMAE (PB101 or LD101) was prepared as follows:

### Step 1

A solution of compound 101-1 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-carbamoylpentyl]carbamate (101-1, 250 mg, 0.108 mmol)), compound 100-2 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-2, 121.64 mg, 0.108 mmol)) and DIPEA (27.95 mg, 0.217 mmol) in anhydrous DMF (4 mL) was stirred at room temperature for 5 min, then a solution of HATU (41.19 mg, 0.108 mmol) in anhydrous DMF (4 mL) was added. The resulting solution was stirred for another 2 h until LCMS indicated a complete reaction. The reaction solution was purified directly by reverse phase flash chromatography (0.01% TFA) to give compound 101-3 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (101-3, 210 mg, 0.061 mmol, 56.75%)) as a white solid. ESI m/z: 854.9 (M/4+H)⁺.

### Step 2

A solution of compound 101-3 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (101-3, 200 mg, 0.059 mmol)) in DMF (3.6 mL) was stirred at room temperature and diethyl amine (0.4 mL, 3.883 mmol) was added. The resulting solution was stirred for 1 h to completion. Then diethyl amine was evaporated with a rotary evaporator, and the residue in DMF was purified by reverse phase flash chromatography (0.01% TFA) to yield the expected product 101-4 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-4, 100 mg, 0.031 mmol, 53.48%)) as a white solid. ESI m/z: 799.5 (M/4+H)⁺.

### Step 3

A solution of compound 101-4 ({4-[(2S)-2-[(2S)-2-[(2S)-2-amino-6-{3-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]-2-[(42S,43R,44R,45R)-42,43,44,45,46-pentahydroxy-40-[(3R,4S,5R)-3,4,5,6-tetrahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-4, 30 mg, 0.009 mmol)) and compound 101-5 (2,5-dioxopyrrolidin-1-yl 6-(2,5-dioxo-2,5-dihydro-1H-pyrrol-1-yl)hexanoate (101-5, 3.47 mg, 0.011 mmol)) in anhydrous DMF (1.5 mL) was stirred at room temperature, then DIPEA (1.74 mg, 0.013 mmol) was added. The resulting solution was stirred at room temperature for 2 h to achieve complete conversion (monitored by LCMS). Then the completed reaction solution was purified directly by Prep-HPLC (0.01% TFA) to get the desired fractions, which were lyophilized to yield PB101 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB101, 21 mg, 0.006 mmol, 66.02%)) as a white solid. ESI m/z: 847.5 (M/4+H)⁺, 1129.6 (M/3+H)⁺. Retention time 6.016 min (HPLC). ¹H NMR (400 MHz, DMSO-d6) δ 10.03 (s, 1H), 8.34-8.10 (m, 4H), 8.06-7.89 (m, 2H), 7.89-7.79 (m, 2H), 7.66-7.62 (m, 1H), 7.59-7.57 (m, 2H), 7.34-7.24 (m, 6H), 7.20-7.13 (m, 1H), 7.00 (s, 2H), 6.02-5.98 (m, 1H), 5.50-5.35 (m, 7H), 5.13-4.78 (m, 8H), 4.70-4.34 (m, 15H), 4.34-4.18 (m, 5H), 4.05-3.93 (m, 6H), 3.83-3.74 (m, 6H), 3.74-3.68 (m, 7H), 3.61-3.40 (m, 116H), 3.26-3.12 (m, 11H), 3.12-2.80 (m, 10H), 2.43-2.36 (m, 3H), 2.32-2.26 (m, 3H), 2.15-1.84 (m, 6H), 1.84-1.45 (m, 15H), 1.45-1.16 (m, 8H), 1.06-0.97 (m, 6H), 0.89-0.73 (m, 24H) ppm. One proton of carboxyl group in TFA was revealed.

Drug linker PB101 can be used to make a conjugate such as PA101.

### Example 33: Preparation of the Drug-Linker (PB102) Containing a PEG Unit and a Cleavable Linker Attached to 6-amino-9-{[4-(aminomethyl)phenyl]methyl}-2-(2-methoxyethoxy)-9H-purin-8-ol

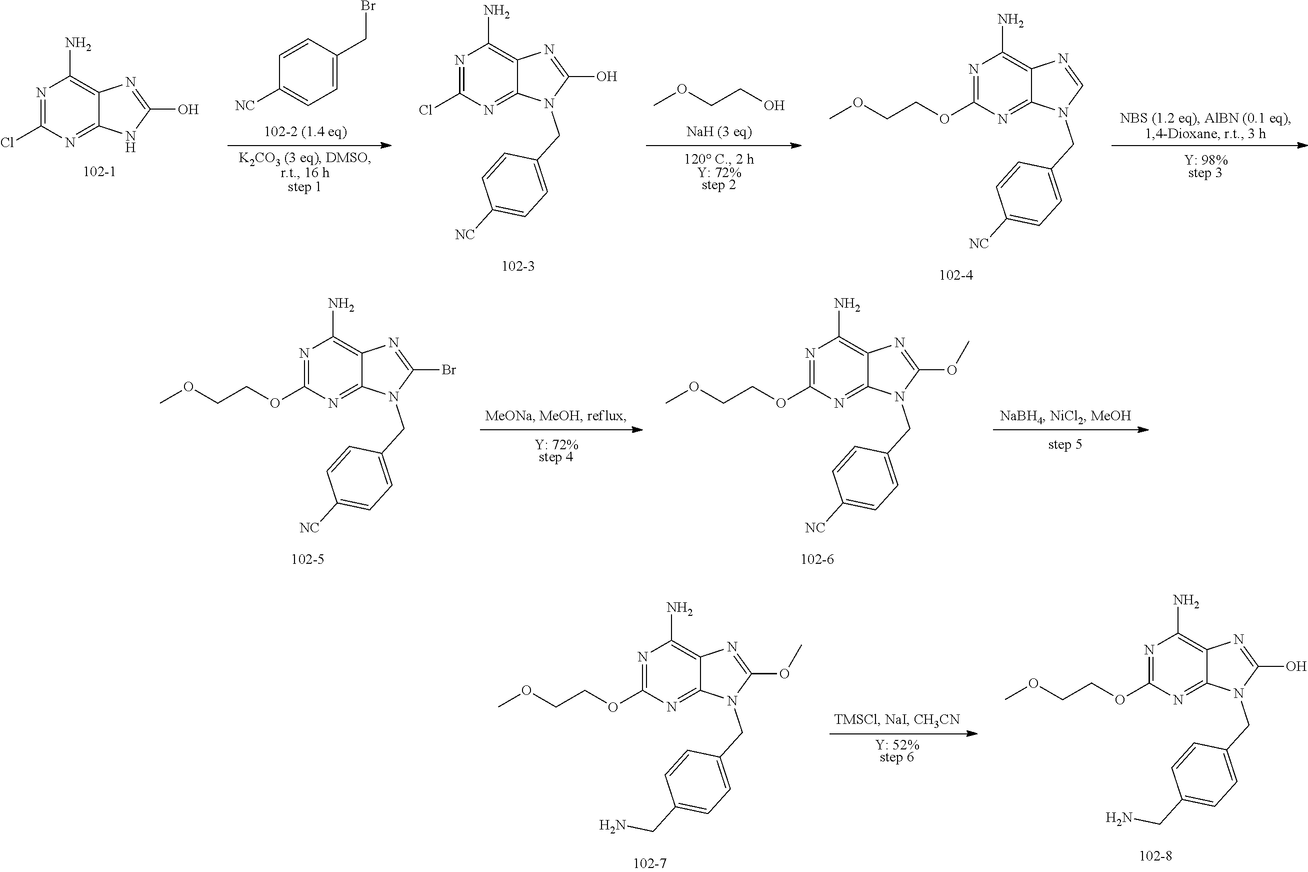

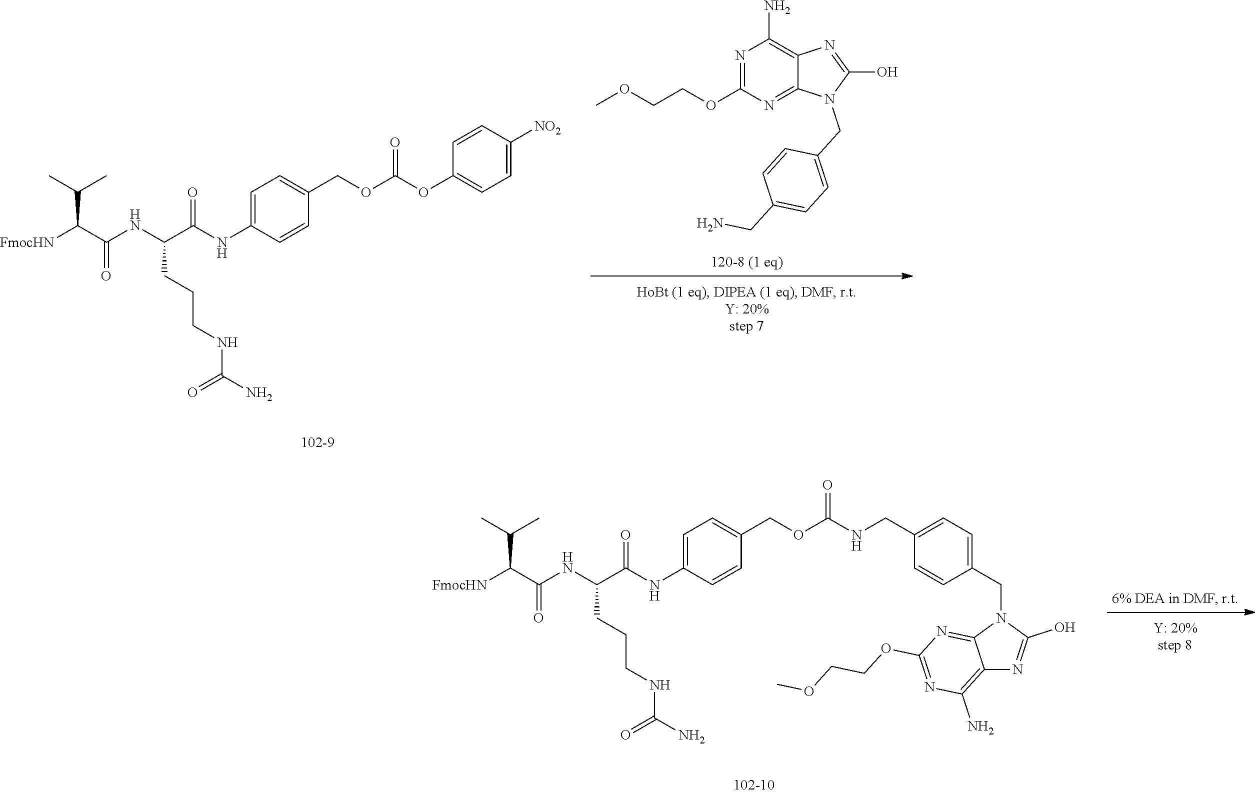

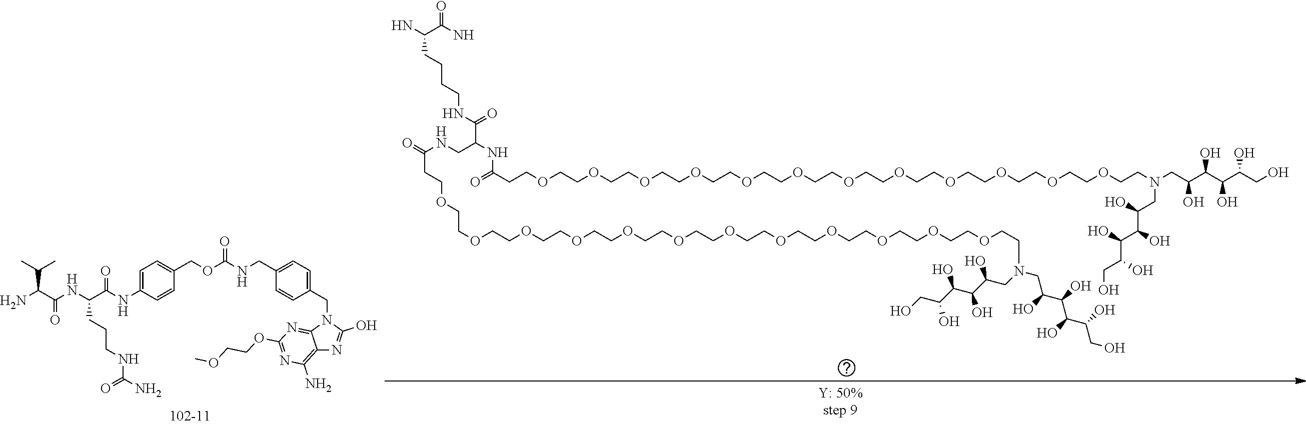 [SMILES: COCCOc1nc(N)c2nc(O)n(Cc3ccc(CNC(=O)OCc4ccc(NC(=O)[C@H](CCCNC(N)=O)NC(=O)[C@@H](N)C(C)C)cc4)cc3)c2n1 ; NC(=O)[C@@H](N)CCCCNC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

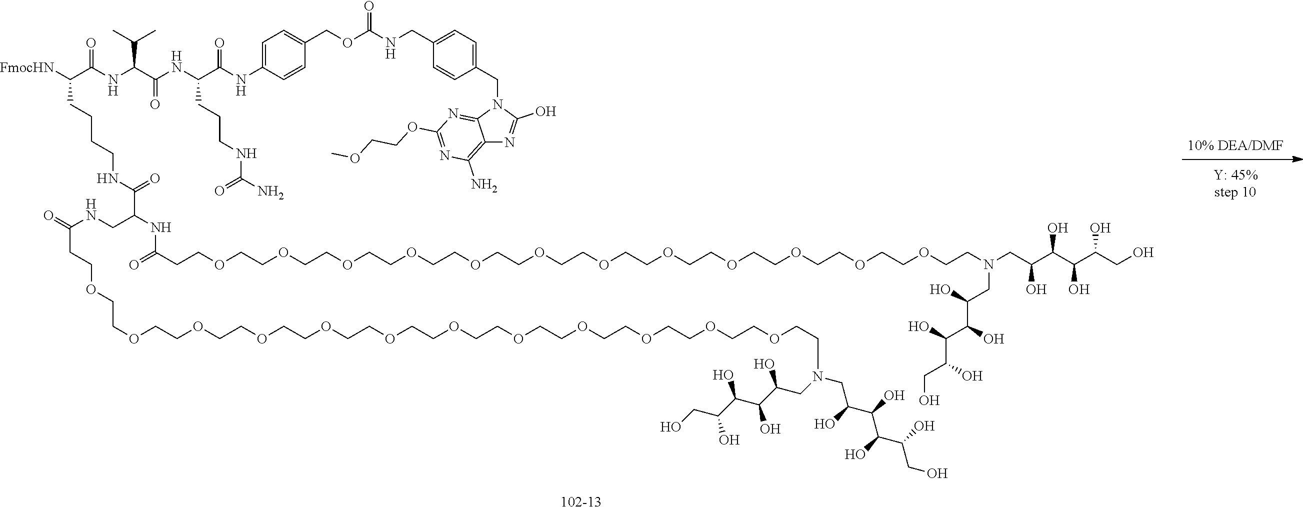

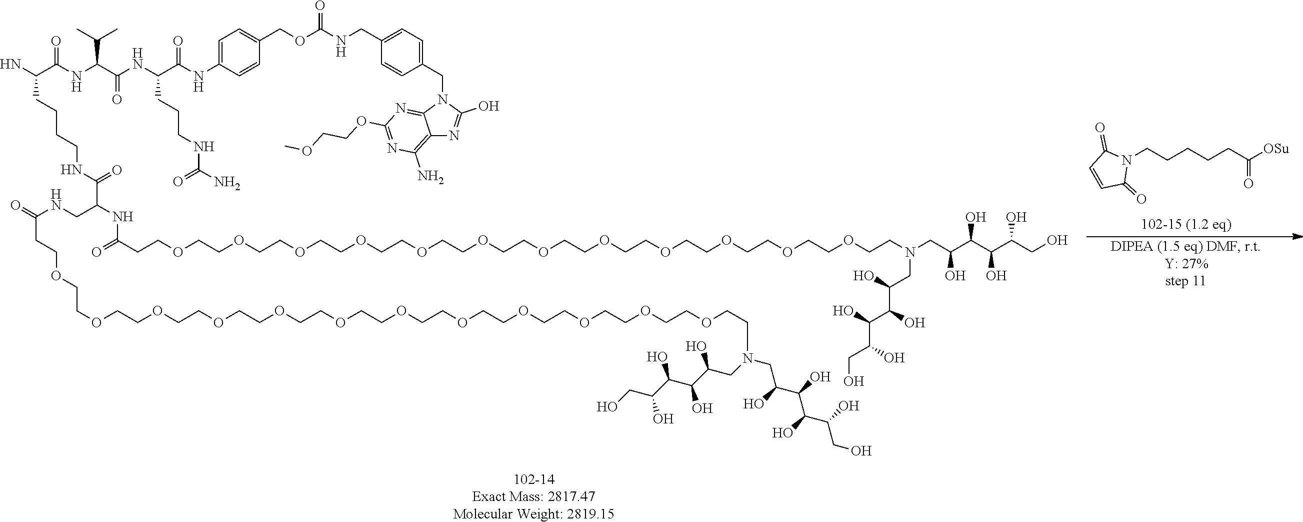

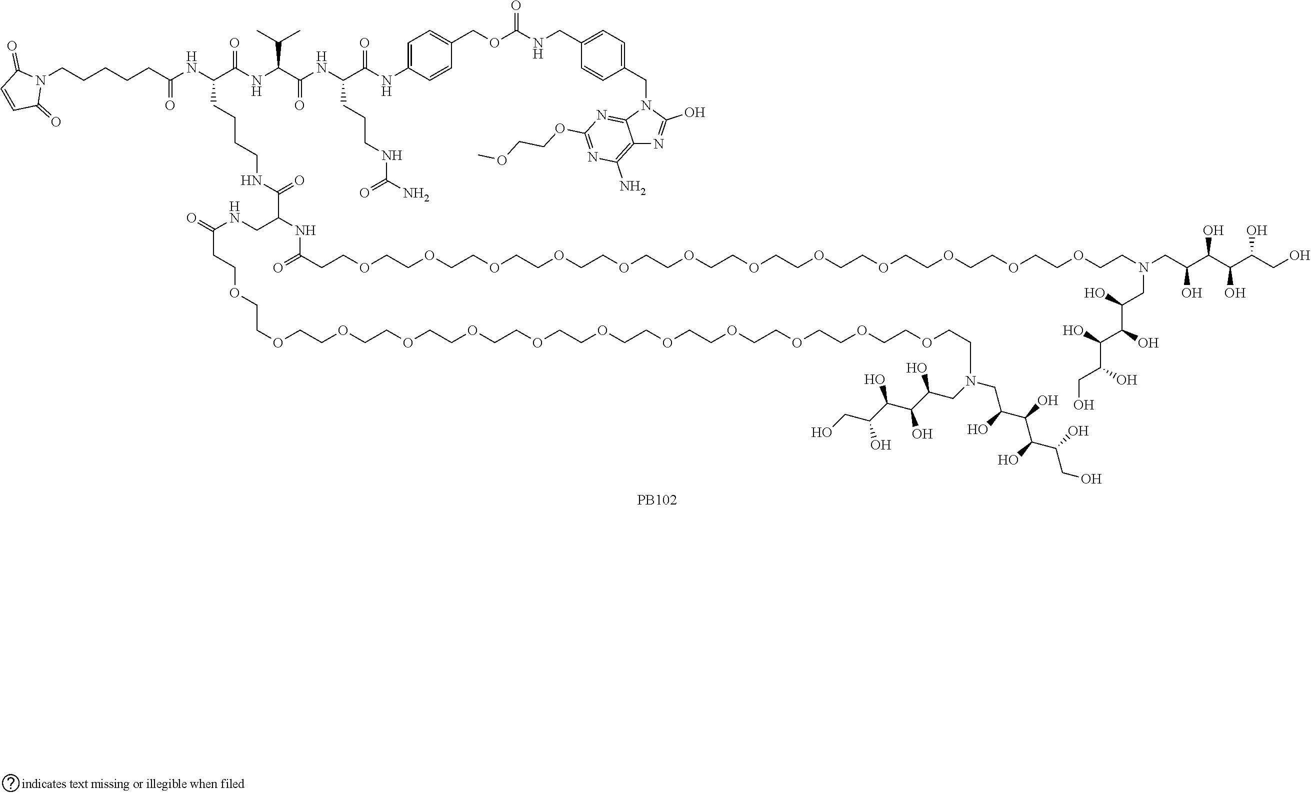

A Drug-Linker containing a PEG unit and a cleavable linker attached to 6-amino-9-{[4-(aminomethyl)phenyl]methyl}-2-(2-methoxyethoxy)-9H-purin-8-ol (PB102) was prepared as follows:

### Step 1

Compound 102-1 (2-chloro-9H-purin-6-amine) (1 eq) and K₂C₃(3 eq) is dissolved in DMSP and the reaction mixture is stirred. 4-(Bromomethyl)benzonitrile (1.4 eq) is added and the reaction mixture is stirred at room temperature for 16 hr. The reaction mixture is filtered to remove insoluble salts and poured into water. The aqueous phase is extracted with EtOAc. The combined organic phases are dried over Mg2SO4 and concentrated in vacuo to afford the product 102-3 (4-((6-amino-2-chloro-9H-purin-9-yl)methyl)benzonitrile (102-3)).

### Step 2

To a solution of NaH (1.24 g, 51.631 mmol) in 2-methoxyethan-1-ol (1.364 mL, 17.210 mmol) was added compound 102-3 (4-[(6-amino-2-chloro-9H-purin-9-yl)methyl]benzonitrile (102-3, 4.9 g, 17.210 mmol)). The mixture was stirred at 80° C. for 2 h. The resulting solution was concentrated and purified by Biotage flash chromatography (silica gel, eluting with 0-10% MeOH in DCM) to afford the product 102-4 (4-[6-amino-2-(2-methoxyethoxy)-9H-purin-9-yl]methyl}benzonitrile (102-4, 4.02 g, 12.394 mmol, 72.04%)) as a pale yellow solid. ESI m/z: 325.0 (M+H)⁺.

### Step 3

To a solution of compound 102-4 (4-{[6-amino-2-(2-methoxyethoxy)-9H-purin-9-yl]methyl}benzonitrile (102-4, 4.02 g, 12.394 mmol)) in 1,4-dioxane (50 mL) was added sulfanylidene-λ\hat{ }4-boranimine (2.65 g, 14.873 mmol) and AIBN (0.183 mL, 1.239 mmol). The mixture was stirred at room temperature for 3 h. The resulting solution was concentrated and purified by flash chromotography (silica gel, eluting with 0-10% MeOH in DCM) to afford the product 102-5 (4-[6-amino-8-bromo-2-(2-methoxyethoxy)-9H-purin-9-yl]methyl}benzonitrile (102-5, 4.90 g, 12.152 mmol, 98%)) as a pale yellow solid. ESI m/z: 404.1 (M+H)⁺.

### Step 4

To a solution of NaOMe (7.37 g, 136.395 mmol) in MeOH (50 mL) was added compound 102-5 (4-{[6-amino-8-bromo-2-(2-methoxyethoxy)-9H-purin-9-yl]methyl}benzonitrile (102-5, 5.5 g, 13.640 mmol)). The mixture was heated under reflux for 3 h. The resulting solution was washed by brine and extracted with DCM (50 mL*3). The organic layer was dried over Na₂SO₄ and evaporated to afford the product 102-6 (4-{[6-amino-8-methoxy-2-(2-methoxyethoxy)-9H-purin-9-yl]methyl}benzonitrile (102-6, 3.5 g, 9.877 mmol, 72.46%). ESI m/z: 355.3 (M+H)⁺).

### Step 5

To the solution of compound 102-6 (4-{[6-amino-8-methoxy-2-(2-methoxyethoxy)-9H-purin-9-yl]methyl}benzonitrile (102-6, 3.5 g, 9.877 mmol)) in MeOH (50 mL) was added NaBH₄ (2.258 mL, 69.137 mmol) and NiCl₂(H₂O)₆ (0.24 g, 0.988 mmol). The mixture was stirred at room temperature for 3 h to completion (monitored by LCMS). The resulting solution was quenched by water and extracted with DCM (50 mL*3). The collected organic layer was dried over Na₂SO₄ and evaporated to afford the crude product 102-7 (9-{[4-(aminomethyl)phenyl]methyl}-8-methoxy-2-(2-methoxyethoxy)-9H-purin-6-amine (102-7, 3.6 g, 10.045 mmol, 101.70%)) as a pale yellow solid which was used directly in next step. ESI m/z: 359.3 (M+H)⁺.

### Step 6

To a solution of compound 102-7 (9-{[4-(aminomethyl)phenyl]methyl}-8-methoxy-2-(2-methoxyethoxy)-9H-purin-6-amine (102-7, 3.5 g, 9.766 mmol)) in MeCN (20 mL) was added ClSiMe₃ (1.06 g, 9.766 mmol) and NaI (0.400 mL, 9.766 mmol). The mixture was stirred at room temperature for 3 h. The resulting solution was concentrated and purified by reverse phase flash chromatography (0.01% TFA) to afford the product 102-8 (6-amino-9-{[4-(aminomethyl)phenyl]methyl}-2-(2-methoxyethoxy)-9H-purin-8-ol (102-8, 1.75 g, 5.082 mmol, 52.08%)) a pale yellow solid. ESI m/z: 345.3 (M+H)⁺, retention time 4.359 min (HPLC). ¹H NMR (400 MHz, DMSO-d6) δ 10.131 (s, 1H), 8.141 (s, 3H), 7.413-7.393 (d, J=8.0 Hz, 2H), 7.340-7.319 (d, J=8.4 Hz, 2H), 6.560 (s, 2H), 4.872 (s, 2H), 4.259-4.235 (t, J=4.8 Hz, 2H), 4.005-3.992 (d, J=5.2 Hz, 2H), 3.593-3.570 (t, J=4.6 Hz, 2H), 3.265 (s, 3H) ppm. One proton of carboxyl group in TFA was revealed.

### Step 7

To a solution of compound 102-8 (6-amino-9-{[4-(aminomethyl)phenyl]methyl}-2-(2-methoxyethoxy)-9H-purin-8-ol (102-8, 800 mg, 2.323 mmol)) in DMF (5 mL) was added compound 102-9 ({4-[(2S)-5-(carbamoylamino)-2-[(2S)-2-({[(9H-fluoren-9-yl)methoxy]carbonyl}amino)-3-methylbutanamido]pentanamido]phenyl}methyl 4-nitrophenyl carbonate (102-9, 1781.31 mg, 2.323 mmol), HOBt (313.89 mg, 2.323 mmol) and DIPEA (300.25 mg, 2.323 mmol). The mixture was stirred at room temperature for 2 h. The resulting solution was purified by reverse phase flash chromatography (0.01% TFA) to afford the product 102-10 ((9H-fluoren-9-yl)methyl N-[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamate (102-10, 450 mg, 0.463 mmol, 19.93%)) as a pale yellow solid. ESI m/z: 972.5 (M+H)⁺.

### Step 8

To a solution of compound 102-10 ((9H-fluoren-9-yl)methyl N-[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamate (102-10, 450 mg, 0.463 mmol)) in DMF (2 mL) was added diethylamine (0.1 mL, 1.276 mmol). The mixture was stirred at room temperature for 1 h. Then the resulting solution was purified by reverse phase flash chromatography (0.01% TFA) to collect the desired fractions, which were lyophilized to afford impure product. The crude product was triturated in acetonitrile and filtered to give cake compound 102-11 ({4-[(2S)-2-[(2S)-2-amino-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-11, 70 mg, 0.093 mmol, 20.17%)) as a pale yellow solid. ESI m/z: 375.8 (M/2+H)⁺.

### Step 9

To a solution of compound 102-11 ({4-[(2S)-2-[(2S)-2-amino-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-11, 58 mg, 0.077 mmol)) in DMF (4 mL) was added compound 102-12 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (102-12, 178.73 mg, 0.077 mmol)), HATU (29.28 mg, 0.077 mmol) and DIPEA (9.95 mg, 0.077 mmol). The resulting solution was stirred at room temperature for 1 h, then was purified by reverse phase flash chromatography (0.01% TFA) to afford the product fractions, which were freeze-dried to yield compound 102-13 ((9H-fluoren-9-yl)methyl N-[(1S)-1-{[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamoyl}-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}pentyl]carbamate (102-13, 120 mg, 0.040 mmol, 51.30%)) as a white solid. ESI m/z: 761.3 (M/4+H)⁺.

### Step 10

To a solution of compound 102-13 ((9H-fluoren-9-yl)methyl N-[(1S)-1-{[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamoyl}-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}pentyl]carbamate (102-13, 120 mg, 0.039 mmol)) in DMF (2 mL) was added diethylamine (0.1 mL, 1.276 mmol). The mixture was stirred at room temperature for 1 h. The resulting solution was purified by reverse phase separation (0.01% TFA) to afford the product 102-14 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-14, 50 mg, 0.018 mmol, 44.95%)) as a white solid. ESI m/z: 706.0 (M/4+H)⁺.

### Step 11

To the solution of compound 102-14 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-14, 50 mg, 0.018 mmol)) in DMF (1 mL) was added compound 102-15 (2,5-dioxopyrrolidin-1-yl 6-(2,5-dioxo-2,5-dihydro-1H-pyrrol-1-yl)hexanoate (102-15, 6.56 mg, 0.021 mmol)) and DIPEA (3.44 mg, 0.027 mmol). The solution was stirred at room temperature for 2 h until all starting amine was consumed. Then the resulting solution was purified directly by Prep-HPLC (0.01% TFA) to afford the product fractions, which were lyophilized to yield PB102 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1H-pyrrol-1-yl)hexanamido]hexanamido]-3-methylbutanamido]-5-(carbamoylamino)pentanamido]phenyl}methyl N—[(4-{[6-amino-8-hydroxy-2-(2-methoxyethoxy)-9H-purin-9-yl]methyl}phenyl)methyl]carbamate (PB102, 20 mg, 0.007 mmol, 37.43%)) as a white solid. ESI m/z: 754.5 (M/4+H)⁺, 1005.1 (M/3+H)⁺, retention time 5.190 min (HPLC). ¹H NMR (400 MHz, DMSO-d6) δ 10.03 (s, 1H), 10.01 (s, 1H), 8.15-8.13 (m, 3H), 7.97 (d, J=8.0 Hz, 1H), 7.94 (d, J=4.0 Hz, 1H), 7.87-7.85 (m, 2H), 7.81-7.75 (m, 1H) 7.66 (d, J=12.0 Hz, 1H), 7.58 (d, J=4.0 Hz, 2H), 7.29-7.19 (m, 6H), 7.00 (s, 2H), 6.50 (s, 2H), 6.01 (t, J=5.6 Hz, 1H), 5.44 (brs, 4H), 4.95 (brs, 2H), 4.82 (brs, 2H), 4.57-4.39 (m, 12H), 4.26-4.14 (m, 8H), 4.05-3.95 (m, 4H), 3.80-3.76 (m, 4H), 3.68-3.61 (m, 4H), 3.58-3.56 (m, 18H), 3.52-3.36 (m, 98H), 3.35-3.29 (m, 12H), 3.26-3.17 (m, 7H), 3.01-2.92 (m, 4H), 2.68-2.66 (m, 1H), 2.39 (t, J=6 Hz, 2H), 2.33-2.29 (m, 2H), 2.20-2.10 (m, 2H), 1.99-1.95 (m, 1H), 1.74-1.55 (m, 3H), 1.46-1.31 (m, 8H), 1.25-1.10 (m, 4H), 0.85-0.81 (m, 6H) ppm. Two protons of carboxyl group in TFA were revealed.

Drug linker PB102 can be used to make a conjugate such as PA102.

### Example 34: Preparation of the Drug-Linker (PB103) Containing a PEG Unit and a Cleavable Linker Attached to Exatecan

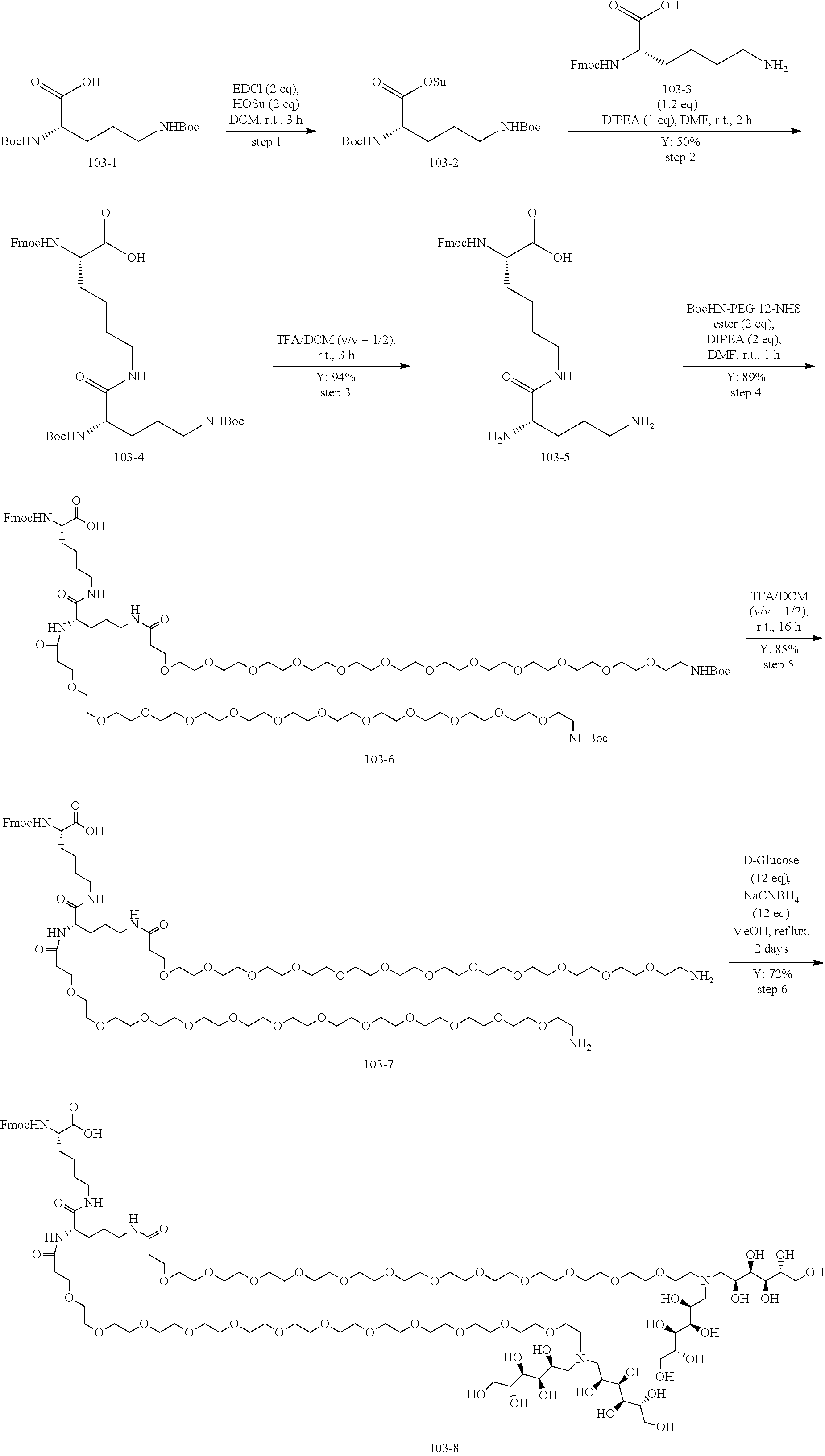

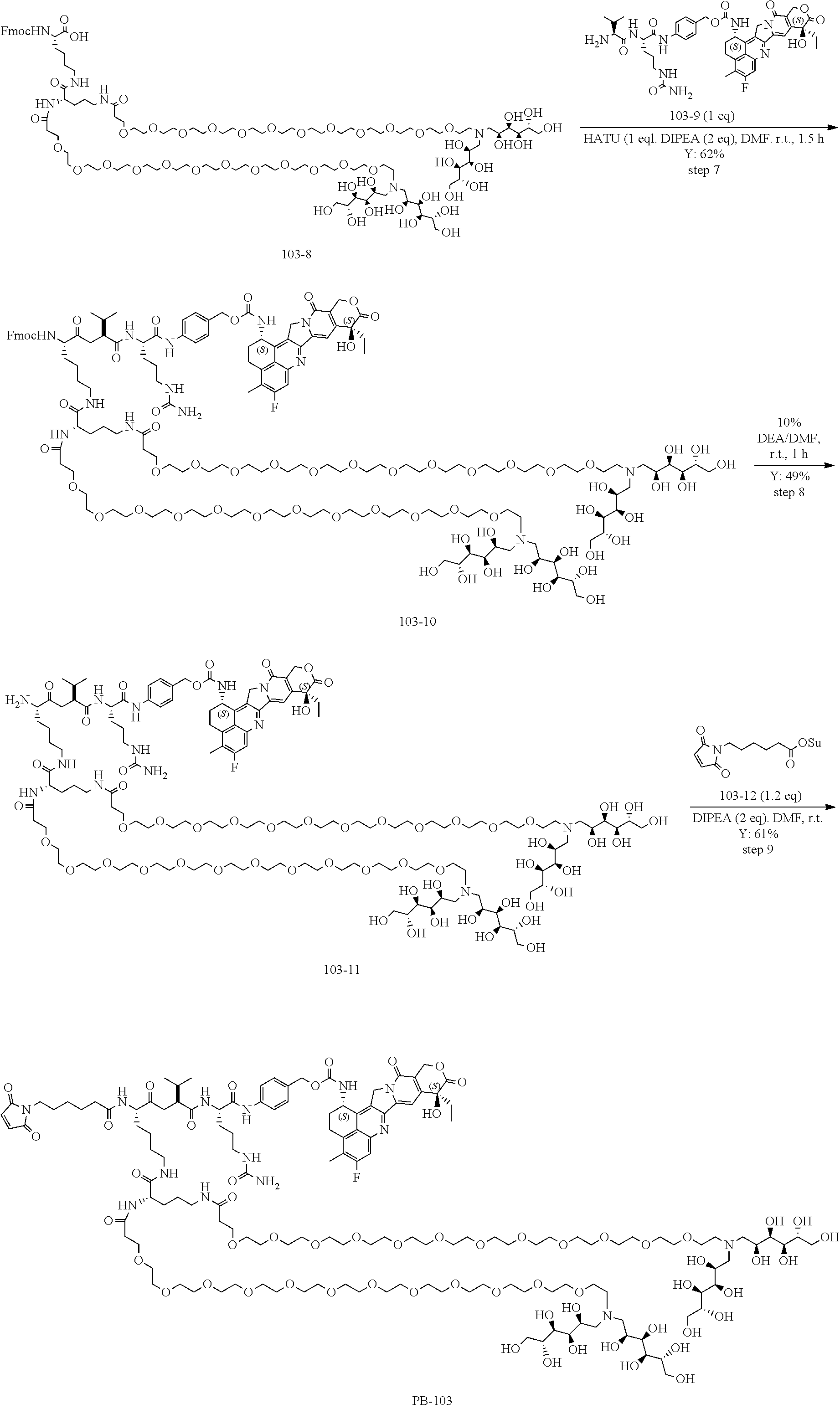

A Drug-Linker containing a PEG unit and a cleavable linker attached to exatecan (PB103) was prepared as follows:

### Step 1

To a solution of compound 103-1 ((2S)-2,5-bis({[(tert-butoxy)carbonyl]amino})pentanoic acid (103-1, 4.5 g, 13.538 mmol)) in DCM (20 mL) was added HOSu (3.12 g, 27.076 mmol) and EDCI (5.19 g, 27.076 mmol). The mixture was stirred at room temperature for 3 h. The resulting solution was washed by water and extracted by DCM. The organic layer was dried over Na₂SO₄ and evaporated to afford the crude product 103-2 (2,5-dioxopyrrolidin-1-yl (S)-2,5-bis((tert-butoxycarbonyl)amino)pentanoate (103-2, 6.0 g, 13.971 mmol, 103.27%)). ESI m/z: 452.3 (M+Na)⁺.

### Step 2

To a solution of compound 103-2 (2,5-dioxopyrrolidin-1-yl (S)-2,5-bis((tert-butoxycarbonyl)amino)pentanoate (103-2, 5.81 g, 13.528 mmol)) in DMF (20 mL) was added compound 103-3 ((2S)-6-amino-2-({[(9H-fluoren-9-yl)methoxy]carbonyl}amino)hexanoic acid (103-3, 5.98 g, 16.234 mmol)) and DIPEA (1.75 g, 13.528 mmol). The mixture was stirred at room temperature for 2 h. The resulting solution was purified by reverse phase flash chromatography (0.01% TFA) to afford the product 103-4 ((2S)-6-[(2S)-2,5-bis({[(tert-butoxy)carbonyl]amino})pentanamido]-2-({[(9H-fluoren-9-yl)methoxy]carbonyl}amino)hexanoic acid (103-4, 8.5 g, 12.448 mmol, 91.99%)) as white solid. ESI m/z: 683.5 (M+H)⁺.

### Step 3

To a solution of compound 103-4 ((2S)-6-[(2S)-2,5-bis({[(tert-butoxy)carbonyl]amino})pentanamido]-2-({[(9H-fluoren-9-yl)methoxy]carbonyl}amino)hexanoic acid (103-4, 8.5 g, 12.448 mmol)) in DCM (20 mL) was added TFA (10 mL, 134.626 mmol). The mixture was stirred at room temperature for 3 h. The resulting solution was evaporated and purified by reverse phase flash chromatography (0.01% TFA) to afford the product 103-5 ((2S)-6-[(2S)-2,5-diaminopentanamido]-2-({[(9H-fluoren-9-yl)methoxy]carbonyl}amino)hexanoic acid (103-5, 5.7 g, 11.812 mmol, 94.84%)) as white solid. ESI m/z: 483.4 (M+H)⁺.

### Step 4

To a solution of 2,5-dioxopyrrolidin-1-yl 2,2-dimethyl-4-oxo-3,8,11,14,17,20,23,26,29,32,35,38,41-tridecaoxa-5-azatetratetracontan-44-oate (5.68 g, 6.970 mmol) in DMF (15 mL) was added DIPEA (0.90 g, 6.970 mmol) and compound 103-5 ((2S)-6-[(2S)-2,5-diaminopentanamido]-2-({[(9H-fluoren-9-yl)methoxy]carbonyl}amino)hexanoic acid (103-5, 1.68 g, 3.485 mmol)). The mixture was stirred at room temperature for 1 h. The resulting solution was purified by reverse phase flash chromatography (0.01% TFA) to afford the product 103-6 ((2S)-6-[(2S)-2,5-bis(1-{[(tert-butoxy)carbonyl]amino}-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-6, 5.9 g, 3.135 mmol, 89.94%)) as transparent oil. ESI m/z: 561.2 ((M−200)/3+H)⁺.

### Step 5

To a solution of compound 103-6 ((2S)-6-[(2S)-2,5-bis(1-{[(tert-butoxy)carbonyl]amino}-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-6, 5.9 g, 3.135 mmol)) in DCM (10 mL) was added TFA (5 mL, 67.313 mmol). The mixture was stirred at room temperature overnight. The resulting solution was concentrated and purified by reverse phase separation (C18 column, eluting with 0-40% acetonitrile in water with TFA) to afford the product 103-7 ((2S)-6-[(2S)-2,5-bis(1-amino-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-7, 4.5 g, 2.675 mmol, 85.39%)) as transparent oil. ESI m/z: 561.6 (M/3+H)⁺.

### Step 6

To a solution of compound 103-7 ((2S)-6-[(2S)-2,5-bis(1-amino-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-7, 4.5 g, 2.675 mmol)) in MeOH (30 mL) was added D-glucose (5.78 g, 32.104 mmol) and NaBH₃CN (1.949 mL, 32.104 mmol). The mixture was stirred at 60° C. over the weekend. The resulting solution was purified by reverse phase flash chromatography (0.01% TFA) to afford the product 103-8 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-({[(9H-fluoren-9-yl)methoxy]carbonyl}amino)hexanoic acid (103-8, 4.53 g, 1.937 mmol, 72.36%)) as a white gel. ESI m/z: 780.4 (M/3+H)⁺.

### Step 7

To a solution of compound 103-8 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-({[(9H-fluoren-9-yl)methoxy]carbonyl}amino)hexanoic acid (103-8, 330 mg, 0.141 mmol)) in DMF (5 mL) was added compound 103-9 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}0.0\hat{ }{20,24}]tetracosa-1,6(11),12,14,16,18,20(24)-heptaen-23-yl]carbamate (103-9, 118.66 mg, 0.141 mmol)), HATU (53.65 mg, 0.141 mmol) and DIPEA (36.47 mg, 0.282 mmol). The mixture was stirred at room temperature for 1.5 h. The resulting solution was adjusted to pH 6 and purified by reverse phase flash chromatography (0.01% TFA) to afford the product 103-10 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[(({[(1S,23S)-10-ethyl-18-fluoro-10-hydroxy-19-methyl-5,9-dioxo-8-oxa-4,15-diazahexacyclo[14.7.1.\hat{ }{2,14}.0\hat{ }{4,13}0.0\hat{ }{6,111}.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (103-10, 273 mg, 0.086 mmol, 61.19%)) as a pale yellow solid. ESI m/z: 790.9 (M/4+H)⁺.

### Step 8

To the solution of compound 103-10 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,111}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (103-10, 273 mg, 0.086 mmol)) in DMF (2.5 mL) was added diethyl amine (0.5 mL, 6.378 mmol). The mixture was stirred at room temperature for 1 h. The resulting solution was purified by reverse phase flash chromatography (0.01% TFA) to afford the product 103-11 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.\hat{ }{2,14}.0\hat{ }{4,13}.\hat{ }{6,11}.0\hat{ }{20,24}]tetracosa-1,6(11),12,14,16,18,20(24)-heptaen-23-yl]carbamate (103-11, 123 mg, 0.042 mmol, 48.46%)) as a pale yellow solid. ESI m/z: 735.7 (M/4+H)⁺, 980.7 (M/3+H)⁺.

### Step 9

To the solution of compound 103-11 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (103-11, 123 mg, 0.042 mmol)) in DMF (3 mL) was added 2,5-dioxopyrrolidin-1-yl 6-(2,5-dioxo-2,5-dihydro-1H-pyrrol-1-yl)hexanoate (103-12, 19.35 mg, 0.063 mmol)) and DIPEA (8.11 mg, 0.063 mmol). The mixture was stirred at room temperature for 1 h. The resulting solution was adjusted to pH 6 and purified by Prep-HPLC (0.01% TFA) to afford the product PB103 ({4-[(2S)-2-[(2S)-2-[(2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (PB103, 80 mg, 0.026 mmol, 61.03%)) as a pale yellow solid. ESI m/z: 627.7 (M/5+H)⁺, 784.0 (M/4+H)⁺, 1044.9 (M/3+H)⁺, retention time 5.614 min (HPLC). ¹H NMR (400 MHz, DMSO-d6) δ 10.05 (s, 1H), 8.16-8.14 (m, 3H), 8.09-8.07 (d, J=8.8 Hz, 1H), 7.99-7.98 (d, J=7.2 Hz, 2H), 7.86-7.83 (m, 2H), 7.80-7.77 (d, J=10.8 Hz, 1H), 7.67-7.65 (d, J=8.0 Hz, 1H), 7.61-7.59 (d, J=8.4 Hz, 2H), 7.37-7.35 (d, J=8.8 Hz, 2H), 7.311 (s, 1H), 7.00 (s, 2H), 6.55 (s, 1H), 6.03-6.01 (t, J=5.2 Hz, 1H), 5.45 (brs, 8H), 5.29-5.23 (m, 3H), 5.07 (s, 2H), 4.82 (brs, 4H), 4.62-4.37 (m, 12H), 4.24-4.16 (m, 3H), 3.98 (brs, 4H), 3.78-3.77 (m, 4H), 3.677 (brs, 4H), 3.61-3.54 (m, 18H), 3.50-3.43 (m, 98H), 3.29-3.23 (m, 7H), 3.17-2.94 (m, 6H), 2.43-2.41 (m, 5H), 2.30-2.26 (m, 3H), 2.24-2.08 (m, 4H), 2.00-1.83 (m, 3H), 1.66-1.59 (m, 4H), 1.49-1.42 (m, 7H), 1.36-1.45 (m, 6H), 1.29-1.14 (m, 4H), 0.89-0.80 (m, 9H) ppm. Drug linker PB103 can be used to make a conjugate such as PA103.

### Example 35: Preparation of the Drug-Linker (PB104) Containing a PEG Unit and a Cleavable Linker Attached to MMAE

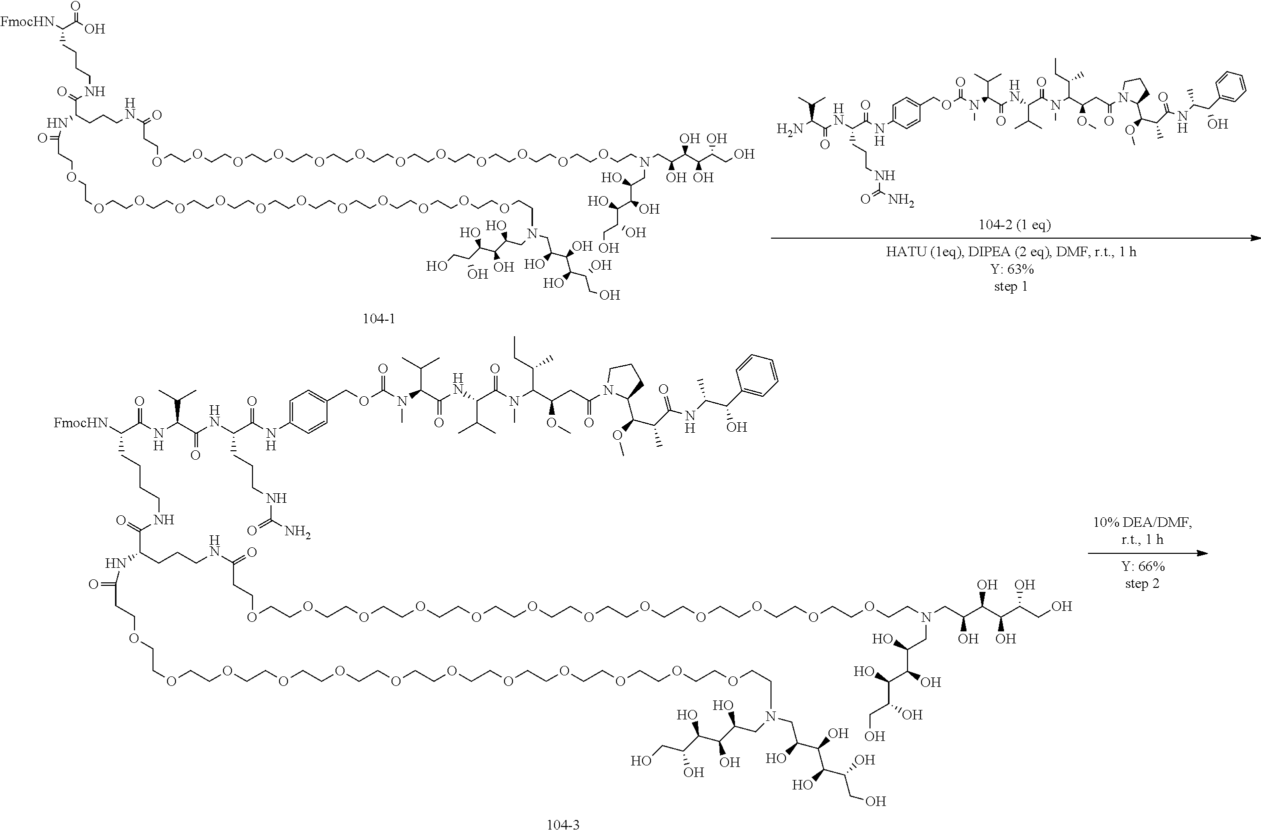

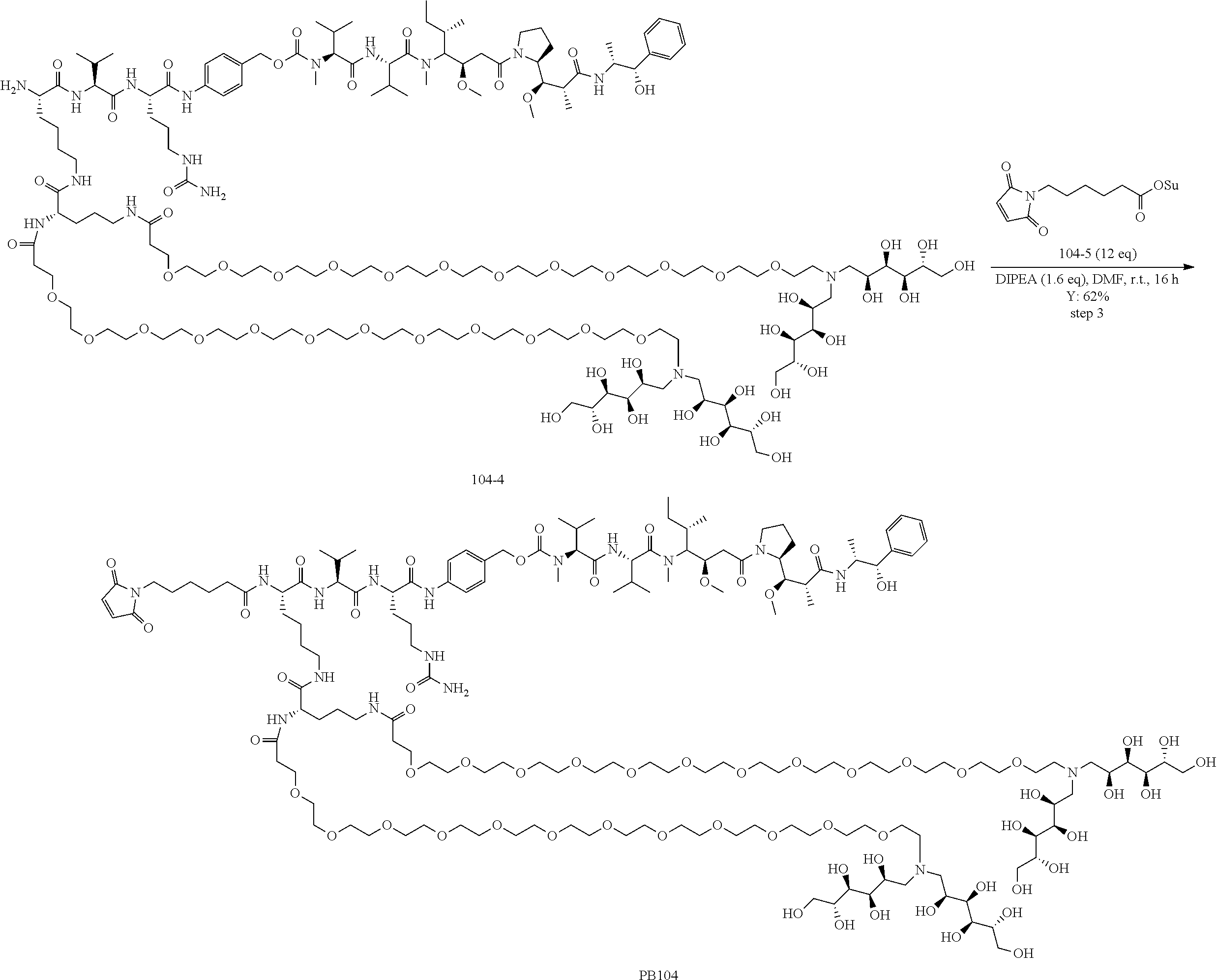

A Drug-Linker containing a PEG unit and a cleavable linker attached to MMAE (PB104) was prepared as follows:

### Step 1

A solution of compound 104-1 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-({[(9H-fluoren-9-yl)methoxy]carbonyl}amino)hexanoic acid (104-1, 300 mg, 0.128 mmol)), compound 104-2 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-2, 144.10 mg, 0.128 mmol)) and DIPEA (33.02 mg, 0.256 mmol) in anhydrous DMF (3 mL) was stirred at room temperature for 5 min, then a solution of HATU (48.79 mg, 0.128 mmol) in anhydrous DMF (1 mL) was added slowly. The resulting solution was stirred for another 1 h until LCMS indicated complete consumption of the starting amine. Then the reaction solution was purified by reverse phase flash chromatography (0.01% TFA) to yield compound 104-3 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (104-3, 280 mg, 0.081 mmol, 63.51%)) as a white solid. ESI m/z: 671.5 ((M−717−26−18)/4+H)⁺, 862.0 (M/4+H)⁺.

### Step 2

A solution of compound 104-3 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (104-3, 260 mg, 0.075 mmol)) in DMF (3.6 mL) was stirred at room temperature and diethyl amine (0.4 mL, 3.883 mmol) was added. The resulting solution was stirred for another 1 h until LCMS showed that the reaction was completed. Volatiles (especially diethyl amine) were evaporated off under vacuo, and the residue was purified by reverse phase column chromatography (0.01% TFA) to yield product 104-4 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-4, 160 mg, 0.050 mmol, 65.78%)) as a white solid. ESI m/z=615.8 ((M−717−26−18)/4+H)⁺, 806.3 (M/4+H)⁺. ¹H NMR (400 MHz, DMSO-d6) δ 10.10 (s, 1H), 8.43 (d, J=8.8 Hz, 1H), 8.35-8.33 (m, 1.5H), 8.17-8.09 (m, 5H), 8.03 (d, J=7.6 Hz, 1H), 7.97-7.78 (m, 3H), 7.66 (d, J=9.2 Hz, 0.5H), 7.59-7.57 (m, 2H), 7.49-7.40 (m, 1H), 7.32-7.24 ((m, 6H), 7.20-7.13 ((m, 1H), 6.04 (d, J=4.2 Hz, 1H), 5.52-5.36 ((m, 7H), 5.11-4.94 ((m, 3H), 4.89-4.69 ((m, 5H), 4.69-4.39 ((m, 16H), 4.30-4.17 (m, 4H), 4.04-3.93 (m, 7H), 3.88-3.81 (m, 2H), 3.79-3.76 (m, 5H), 3.72-3.62 (m, 6H), 3.62-3.49 (m, 88H), 3.43-3.25 (m, 20H), 3.25-3.06 (m, 12H), 3.06-2.83 (m, 11H), 2.40-2.38 (m, 2H), 2.30-2.22 (m, 3H), 2.14-1.92 (m, 4H), 1.84-1.62 (m, 6H), 1.62-1.21 (m, 16H), 1.05-0.97 (m, 6H), 0.97-0.75 (m, 26H) ppm. Three protons of carboxyl groups in TFA were revealed.

### Step 3

A solution of compound 104-4 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-4, 100 mg, 0.029 mmol) and DIPEA (5.65 mg, 0.044 mmol)) in anhydrous DMF (1.5 mL) was stirred at room temperature for 5 min, then a solution of compound 104-5 (2,5-dioxopyrrolidin-1-yl 6-(2,5-dioxo-2,5-dihydro-1H-pyrrol-1-yl)hexanoate (104-5, 10.80 mg, 0.035 mmol)) in anhydrous DMF (0.5 mL) was added dropwise by syringe over 5 min. After addition, the resulting solution was stirred at room temperature for overnight for convenience, and LCMS indicated complete reaction in the morning. The resulting solution was purified directly by Prep-HPLC (0.01% TFA) to yield PB104 ({4-[(2S)-2-[(2S)-2-[(2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB104, 55 mg, 0.016 mmol, 55.11%)) as a white solid. ESI m/z: 854.5 (M/4+H)⁺; retention time 5.614 min (HPLC). ¹H NMR (400 MHz, DMSO-d6) δ 10.03 (s, 1H), 8.34-8.10 (m, 4H), 8.00-7.97 (m, 2H), 7.93-7.83 (m, 2.5H), 7.67-7.64 (m, 1.5H), 7.58 (d, J=8.0 Hz, 2H), 7.34-7.25 (m, 6H), 7.19-7.13 (m, 1H), 7.00 ((s, 2H), 6.02-5.99 ((m, 1H), 5.52-5.38 ((m, 7H), 5.14-4.94 ((m, 3H), 4.94-4.18 ((m, 25H), 4.04-3.93 (m, 7H), 3.79-3.77 (m, 5H), 3.69-3.66 (m, 5H), 3.62-3.56 (m, 20H), 3.52-3.47 (m, 88H), 3.36-3.12 (m, 15H), 3.03-2.83 (m, 13H), 2.43-2.37 (m, 3H), 2.30-2.27 (m, 3H), 2.15-2.06 (m, 4H), 2.01-1.94 (m, 2H), 1.81-1.50 (m, 8H), 1.50-1.17 (m, 20H), 1.05-0.97 (m, 6H), 0.89-0.75 (m, 26H) ppm. Two protons of carboxyl groups in TFA were revealed. Drug linker PB104 can be used to make a conjugate such as PA104.

### Example 36: Preparation of the Drug-Linker (PB105) Containing a PEG Unit and a Cleavable Linker Attached to MMAE

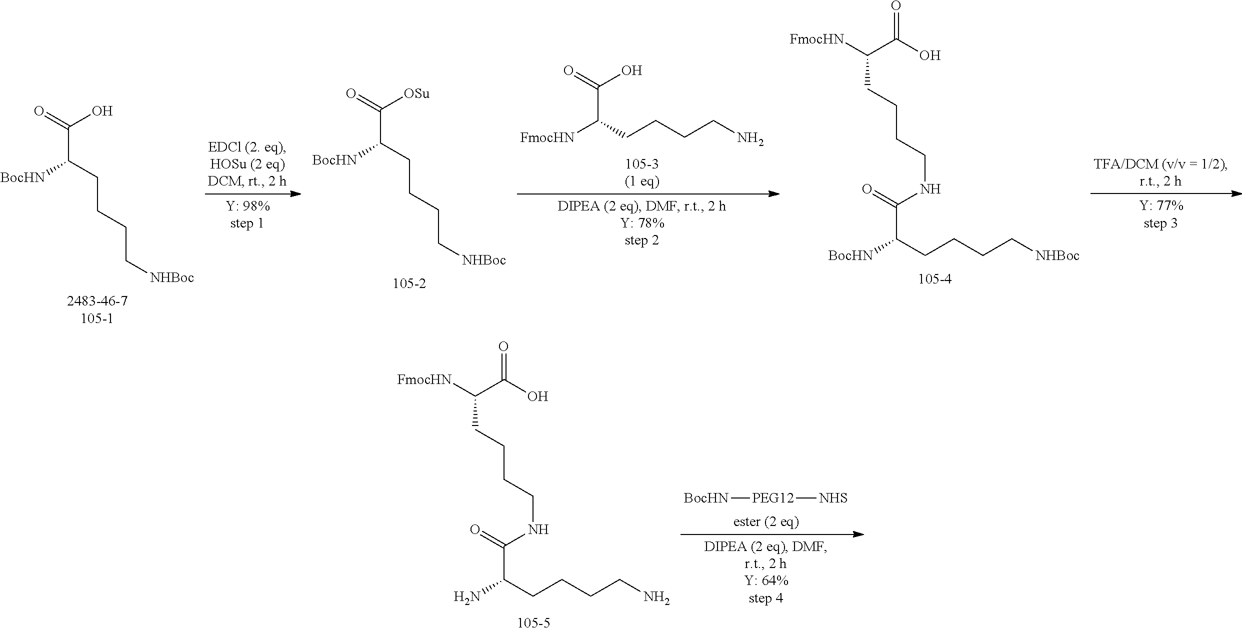

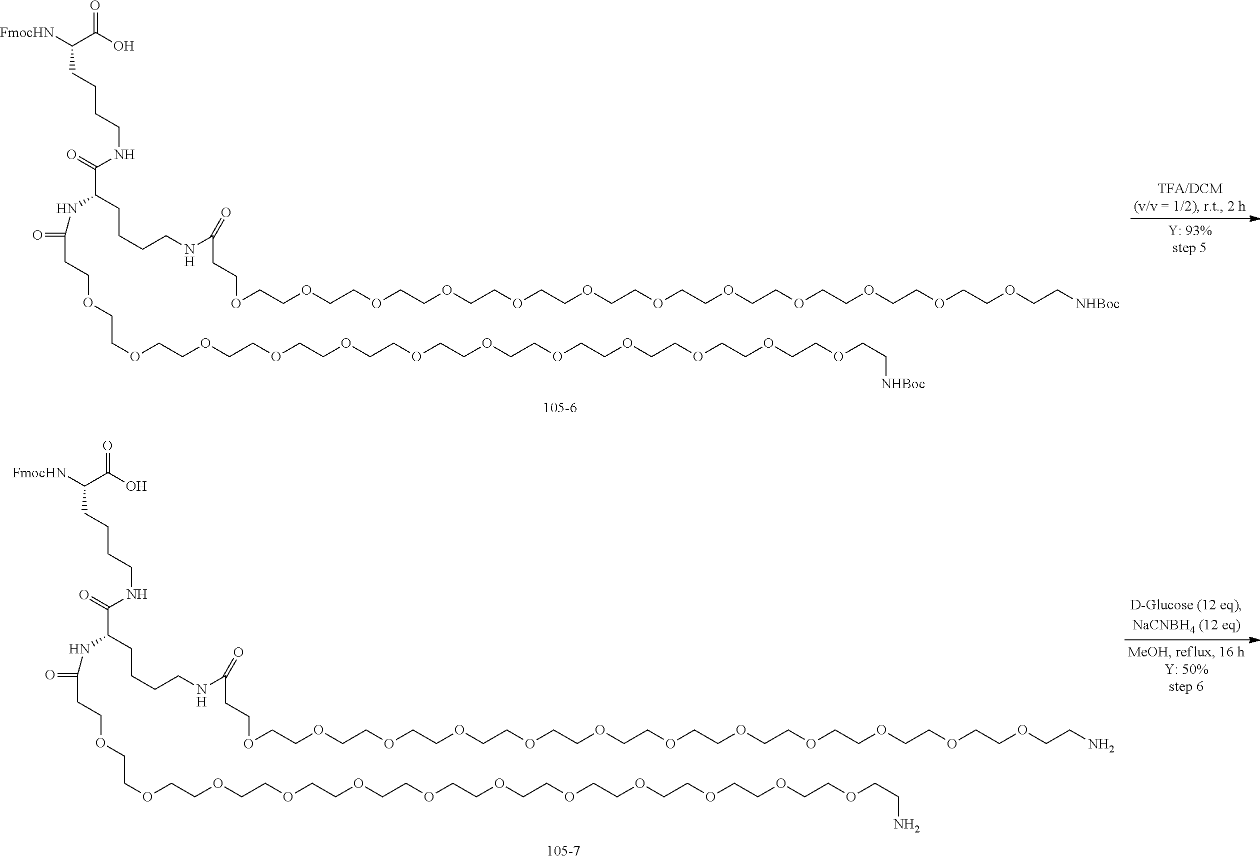

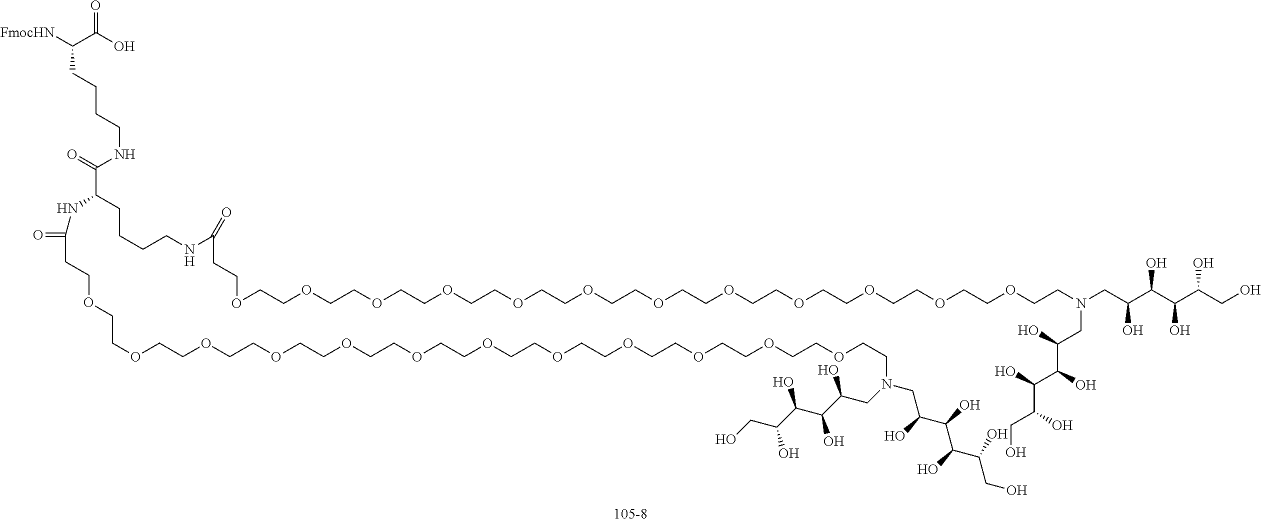

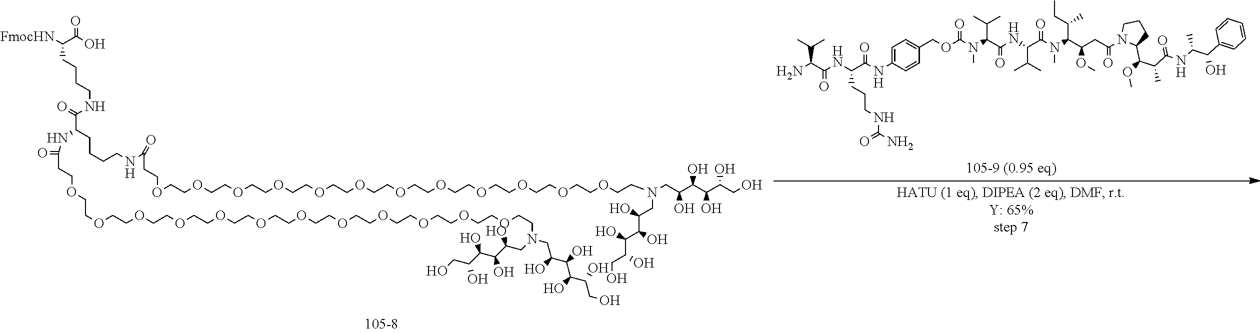

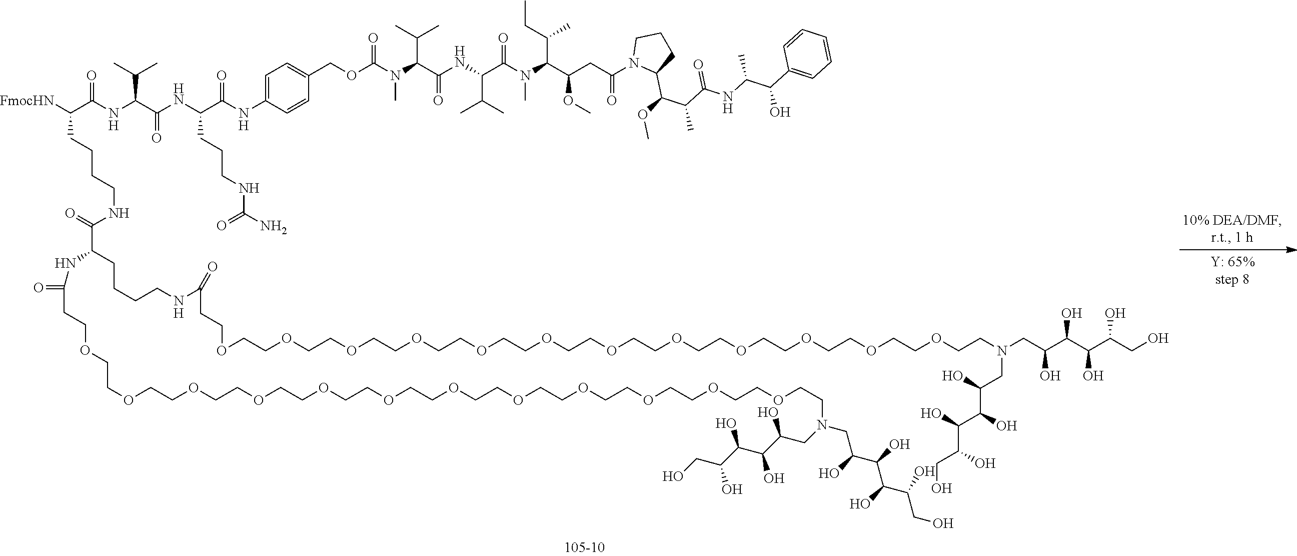

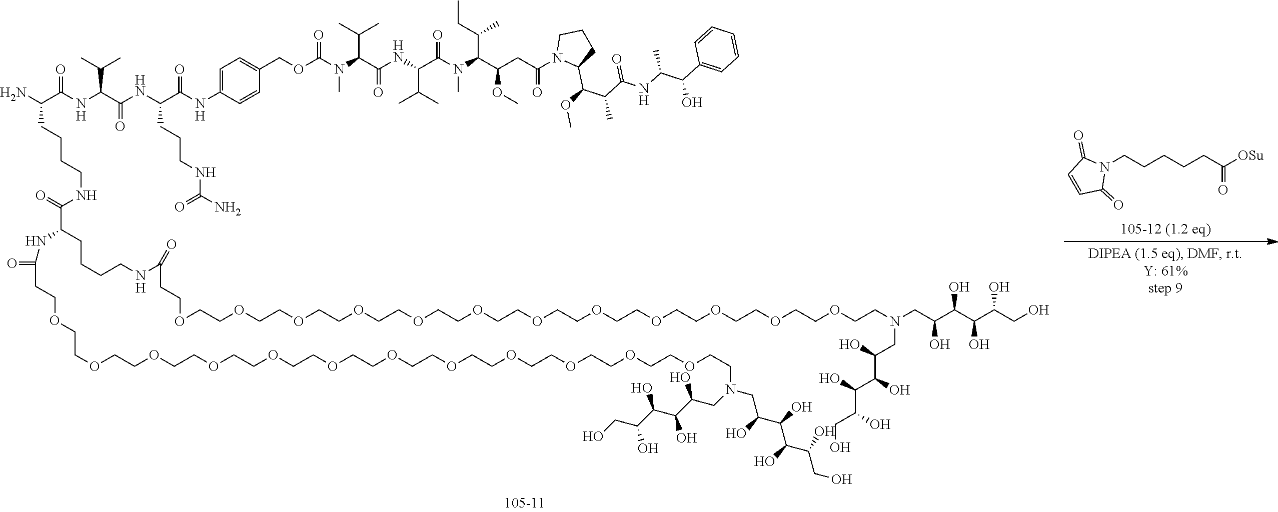

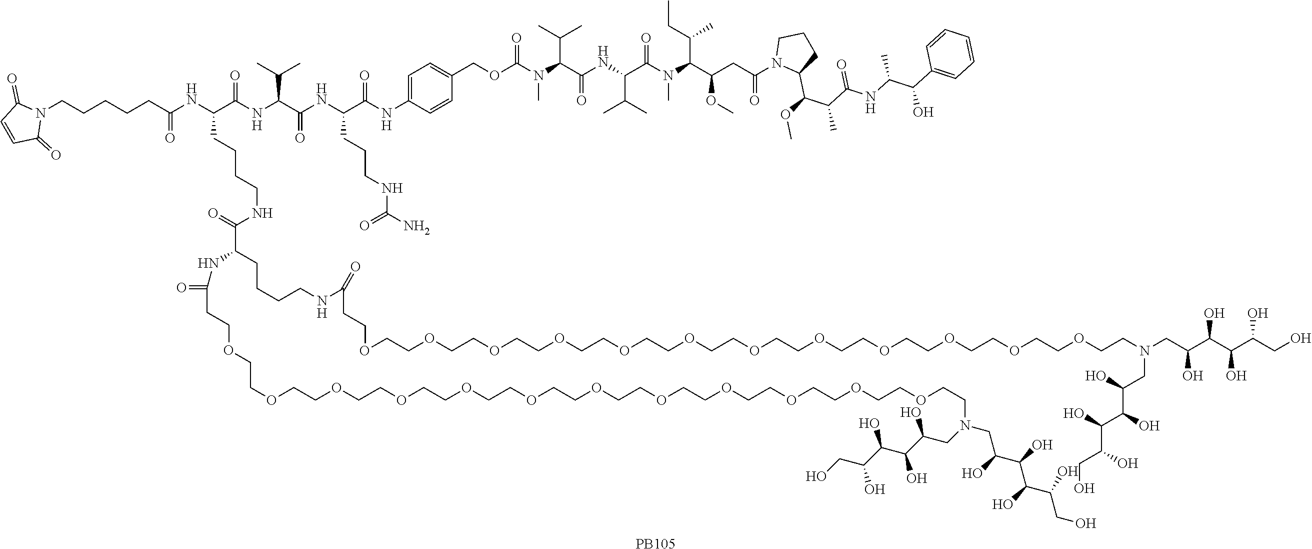

A Drug-Linker containing a PEG unit and a cleavable linker attached to MMAE (PB105) was prepared as follows:

### Step 1

A solution of compound 105-1 ((2S)-2,6-bis({[(tert-butoxy)carbonyl]amino})hexanoic acid (105-1, 5.0 g, 14.433 mmol)), 1-hydroxypyrrolidine-2,5-dione (2.249 mL, 28.867 mmol) and EDCI (5.53 g, 28.867 mmol) in DCM (50 mL) was stirred at room temperature for 2 h. Then the reaction solution was diluted with DCM (50 mL) and washed with water (50 mL*2). The organic layer was collected and dried over sodium sulfate, filtered and the filtrate concentrated under vacuo to dryness, affording crude compound 105-2 ((S)-2,5-dioxopyrrolidin-1-yl 2,6-bis(tert-butoxycarbonylamino)hexanoate (105-2, 6.35 g, 14.318 mmol, 99.20%)), which was used for the next steps directly. ESI m/z: 466.3 (M+Na)⁺.

### Step 2

A solution of compound 105-2 ((S)-2,5-dioxopyrrolidin-1-yl 2,6-bis(tert-butoxycarbonylamino)hexanoate (105-2, 6.40 g, 14.44 mmol)), compound 105-3 ((2S)-6-amino-2-({[(9H-fluoren-9-yl)methoxy]carbonyl}amino)hexanoic acid (105-3, 5.32 g, 14.440 mmol)) and DIPEA (3.73 g, 28.880 mmol) in DMF (10 mL) was stirred at room temperature for 2 h. Then the reaction mixture was purified by reverse phase flash chromatography (0.01% TFA) to get the desired fractions, which were freeze-dried to yield compound 105-4 ((2S)-6-[(2S)-2,6-bis({[(tert-butoxy)carbonyl]amino})hexanamido]-2-({[(9H-fluoren-9-yl)methoxy]carbonyl}amino)hexanoic acid (105-4, 7.9 g, 11.337 mmol, 78.51%)) as a white solid. ESI m/z: 719.4 (M+Na)⁺.

### Step 3

To a solution of compound 105-4 ((2S)-6-[(2S)-2,6-bis({[(tert-butoxy)carbonyl]amino})hexanamido]-2-({[(9H-fluoren-9-yl)methoxy]carbonyl}amino)hexanoic acid (105-4, 4.53 g, 6.501 mmol)) in DCM (20 mL) was added TFA (10 mL, 134.626 mmol) slowly. The mixture was stirred at room temperature for 2 hour. Then the reaction solution was concentrated and the crude mixture was purified by reverse phase flash chromatography (0.01% TFA) to yield compound 105-5 ((2S)-6-[(2S)-2,6-diaminohexanamido]-2-({[(9H-fluoren-9-yl)methoxy]carbonyl}amino)hexanoic acid (105-5, 2.5 g, 5.034 mmol, 77.44%)) as a colorless oil. ESI m/z: 497.3 (M+H)⁺.

### Step 4

A solution of crude compound 105-5 ((2S)-6-[(2S)-2,6-diaminohexanamido]-2-({[(9H-fluoren-9-yl)methoxy]carbonyl}amino)hexanoic acid (105-5, 1.94 g, 3.907 mmol)) in DMF (5 mL) was added dropwise to a solution of 2,5-dioxopyrrolidin-1-yl 1-{[(tert-butoxy)carbonyl]amino}-3,6,9,12,15,18,21,24,27,30,33,36-dodecaoxanonatriacontan-39-oate (5.7 g, 6.995 mmol) and DIPEA (1.01 g, 7.813 mmol) in DMF (10 mL). The resulting solution was stirred at room temperature for 2 hr to completion. The reaction mixture was purified by reverse phase flash chromatography (0.01% TFA) to get the desired fractions, which were freeze-dried to yield compound 105-6 ((2S)-6-[(2S)-2,6-bis(1-{[(tert-butoxy)carbonyl]amino}-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-6, 4.75 g, 2.505 mmol, 64.12%)) as a white solid. ESI m/z: 566.2 (M−200)/3+H)⁺, 970.6 (M/2+Na)⁺.

### Step 5

To a solution of compound 105-6 ((2S)-6-[(2S)-2,6-bis(1-{[(tert-butoxy)carbonyl]amino}-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-6, 4.75 g, 2.505 mmol)) in DCM (10 mL) was added TFA (5 mL, 67.313 mmol). Then the solution was stirred at room temperature for 2 hours, concentrated to remove organic solvent, and the crude residue was purified by reverse phase flash chromatography (0.01% TFA) to yield compound 105-7 ((2S)-6-[(2S)-2,6-bis(1-amino-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-7, 3.95 g, 2.329 mmol, 92.97%)) as a colorless oil. ESI m/z: 566.3 (M/3+H)⁺.

### Step 6

To a solution of compound 105-7 ((2S)-6-{[(2S)-2,6-bis(1-amino-3,6,9,12,15,18,21,24,27,30,33,36-dodecaoxanonatriacontan-39-amido)hexyl]amino}-2-({[(9H-fluoren-9-yl)methoxy]carbonyl}amino)hexanoic acid (105-7, 3.95 g, 2.329 mmol)) in MeOH (90 mL) was added D-glucose (5.04 g, 27.948 mmol) in portions, and the mixture was heated under reflux for 30 minutes under a N₂ atmosphere. Then a solution of NaCNBH₃ (1.76 g, 27.948 mmol) in MeOH (10 mL) was added dropwise. The reaction mixture was stirred at this temperature for 18 h. Then the reaction solution was purified by reverse phase flash chromatography (0.01% TFA) to get the desired fractions, which were freeze-dried to yield compound 105-8 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (105-8, 4.13 g, 1.755 mmol, 75.36%)) as a colorless oil. ESI m/z: 785.0 (M/3+H)⁺.

### Step 7

A solution of compound 105-8 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (105-8, 226.17 mg, 0.096 mmol)), compound 105-9 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (105-9, 90 mg, 0.080 mmol)) and DIPEA (20.64 mg, 0.160 mmol) in anhydrous DMF (3 mL) was stirred at room temperature for 5 min to allow the starting acid to dissolve thoroughly in solvent, then a solution of HATU (36.55 mg, 0.096 mmol) in anhydrous DMF (1 mL) was added dropwise by syringe over 10 min. After addition, the resulting solution was stirred for another 2 h to achieve complete reaction. Then the reaction solution was purified directly by reverse phase flash chromatography (0.01% TFA) to give the desired fractions, which were lyophilized by LabConc to yield a TFA salt of compound 105-10 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (105-10, 180 mg, 0.052 mmol, 65.06%)) as a white solid. ESI m/z: 865.5 (M/4+H)⁺. ¹H NMR (400 MHz, DMSO-d₆) δ 10.07 (s, 1H), 8.36-8.08 (m, 4H), 7.98-7.88 (m, 5H), 7.83-7.79 (m, 1H), 7.74-7.65 (m, 3H), 7.58-7.56 (m, 3H), 7.43-7.39 (m, 2H), 7.34-7.24 (m, 8H), 7.20-7.13 (m, 1H), 6.00 (t, J=5.2 Hz, 1H), 5.51-5.36 (m, 6H), 5.09-4.94 (m, 2H), 4.94-4.70 (m, 4H), 4.70-4.38 (m, 14H), 4.33-4.14 (m, 7H), 4.04-3.93 (m, 6H), 3.79-3.73 (m, 4H), 3.68-3.64 (m, 4H), 3.61-3.55 (m, 18H), 3.52-3.46 (m, 96H), 3.25-3.12 (m, 13H), 3.06-2.83 (m, 13H), 2.43-2.37 (m, 3H), 2.30-2.26 (m, 3H), 2.16-2.04 (m, 2H), 2.04-1.91 (m, 2H), 1.83-1.15 (m, 25H), 1.05-0.97 (m, 6H), 0.97-0.73 (m, 26H) ppm. One proton of carboxyl group in TFA was revealed.

### Step 8

A solution of compound 105-10 (4-((2S,5S,8S,15S,62S,63R,64R,65R)-8-((((9H-fluoren-9-yl)methoxy)carbonyl)amino)-62,63,64,65,66-pentahydroxy-5-isopropyl-4,7,14,21-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-60-((2S,3R,4R,5R)-2,3,4,5,6-pentahydroxyhexyl)-2-(3-ureidopropyl)-24,27,30,33,36,39,42,45,48,51,54,57-dodecaoxa-3,6,13,20,60-pentaazahexahexacontanamido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (105-10, 180 mg, 0.052 mmol)) in DMF (1.8 mL) was stirred at room temperature and diethylamine (0.2 mL, 1.941 mmol) was added. The resulting solution was stirred for 1 h until the reaction was completed. Most of diethyl amine was evaporated off under vacuo and the residue was purified by reverse phase flash chromatography (0.01% TFA) to give the expected fractions, which were lyophilized to yield a TFA salt of compound 105-11 (4-((2S,5S,8S,15S,62S,63R,64R,65R)-8-amino-62,63,64,65,66-pentahydroxy-5-isopropyl-4,7,14,21-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-60-((2S,3R,4R,5R)-2,3,4,5,6-pentahydroxyhexyl)-2-(3-ureidopropyl)-24,27,30,33,36,39,42,45,48,51,54,57-dodecaoxa-3,6,13,20,60-pentaazahexahexacontanamido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (105-11, 110 mg, 0.034 mmol, 65.31%)) as a white solid. ESI m/z: 619.1 (linker fragment, (M−717−26−18)/4+H))⁺, 809.9 (M/4+H)⁺. ¹H NMR (400 MHz, DMSO-d₆) δ 10.10 (s, 1H), 8.43 (d, J=8.4 Hz, 1H), 8.36-8.33 (m, 1H), 8.15-8.10 (m, 5H), 8.01 (d, J=7.6 Hz, 1H), 7.94-7.91 (m, 1.5H), 7.85-7.83 (m, 1H), 7.67 (d, J=8.4 Hz, 0.5H), 7.59-7.57 (m, 2H), 7.35-7.24 (m, 6H), 7.20-7.13 (m, 1H), 6.07-6.05 (m, 1H), 5.53-5.38 (m, 6H), 5.09-4.94 (m, 2H), 4.94-4.42 (m, 18H), 4.30-4.13 (m, 4H), 4.04-3.93 (m, 6H), 3.85-3.81 (m, 1H), 3.81-3.73 (m, 5H), 3.73-3.64 (m, 5H), 3.61-3.55 (m, 18H), 3.55-3.48 (m, 88H), 3.39-3.29 (m, 10H), 3.24-3.12 (m, 12H), 3.06-2.83 (m, 11H), 2.44-2.38 (m, 3H), 2.38-2.22 (m, 4H), 2.15-2.05 (m, 2H), 2.05-1.94 (m, 2H), 1.85-1.64 (m, 6H), 1.64-1.41 (m, 6H), 1.41-1.16 (m, 12H), 1.05-0.97 (m, 6H), 0.97-0.75 (m, 26H) ppm. Three protons of carboxyl group in TFA were revealed.

### Step 9

A solution of compound 105-11 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (105-11, 100 mg, 0.031 mmol)) and DIPEA (7.97 mg, 0.062 mmol) in anhydrous DMF (2 mL) was stirred at room temperature, then a solution of 2,5-dioxopyrrolidin-1-yl 6-(2,5-dioxo-2,5-dihydro-1H-pyrrol-1-yl)hexanoate (11.43 mg, 0.037 mmol) in anhydrous DMF (2 mL) was added dropwise by syringe over 5 min. After addition, the resulting solution was stirred for another 6 h until the starting amine was substantially consumed. Then the resulting solution was purified directly by Prep-HPLC (0.01% TFA) to yield a TFA salt of PB105 ({4-[(2S)-2-[(2S)-2-[(2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB105, 65 mg, 0.019 mmol, 61.34%)) as a white solid. ESI m/z: 667.8 (linker fragment, (M−717−26−18)/4+H)⁺; 858.3 (M/4+H)⁺. Retention time 6.042 min (HPLC). ¹H NMR (400 MHz, DMSO-d6) δ 10.05 (s, 1H), 8.38-8.30 (m, 0.5H), 8.17-8.10 (m, 3.5H), 7.99-7.91 (m, 2.5H), 7.91-7.82 (m, 2H), 7.68-7.65 (m, 1.5H), 7.59-7.57 (m, 2H), 7.34-7.24 (m, 6H), 7.19-7.15 (m, 1H), 7.00 (s, 2H), 6.08-5.96 (m, 1H), 5.53-5.37 (m, 6H), 5.14-4.36 (m, 22H), 4.29-4.13 (m, 5H), 4.04-3.93 (m, 7H), 3.82-3.73 (m, 5H), 3.73-3.66 (m, 5H), 3.62-3.55 (m, 20H), 3.52-3.41 (m, 88H), 3.36-3.24 (m, 10H), 3.24-3.12 (m, 12H), 3.12-2.83 (m, 11H), 2.43-2.35 (m, 3H), 2.29 (t, J=6.8 Hz, 2H), 2.14-1.92 (m, 6H), 1.83-1.46 (m, 16H), 1.46-1.16 (m, 12H), 1.05-0.97 (m, 6H), 0.89-0.75 (m, 26H) ppm. Two protons of carboxyl group in TFA were revealed. Drug linker PB105 can be used to make a conjugate such as PA105.

### Example 37: Preparation of the Drug-Linker (PB106) Containing a PEG Unit and a Cleavable Linker Attached to Exatecan

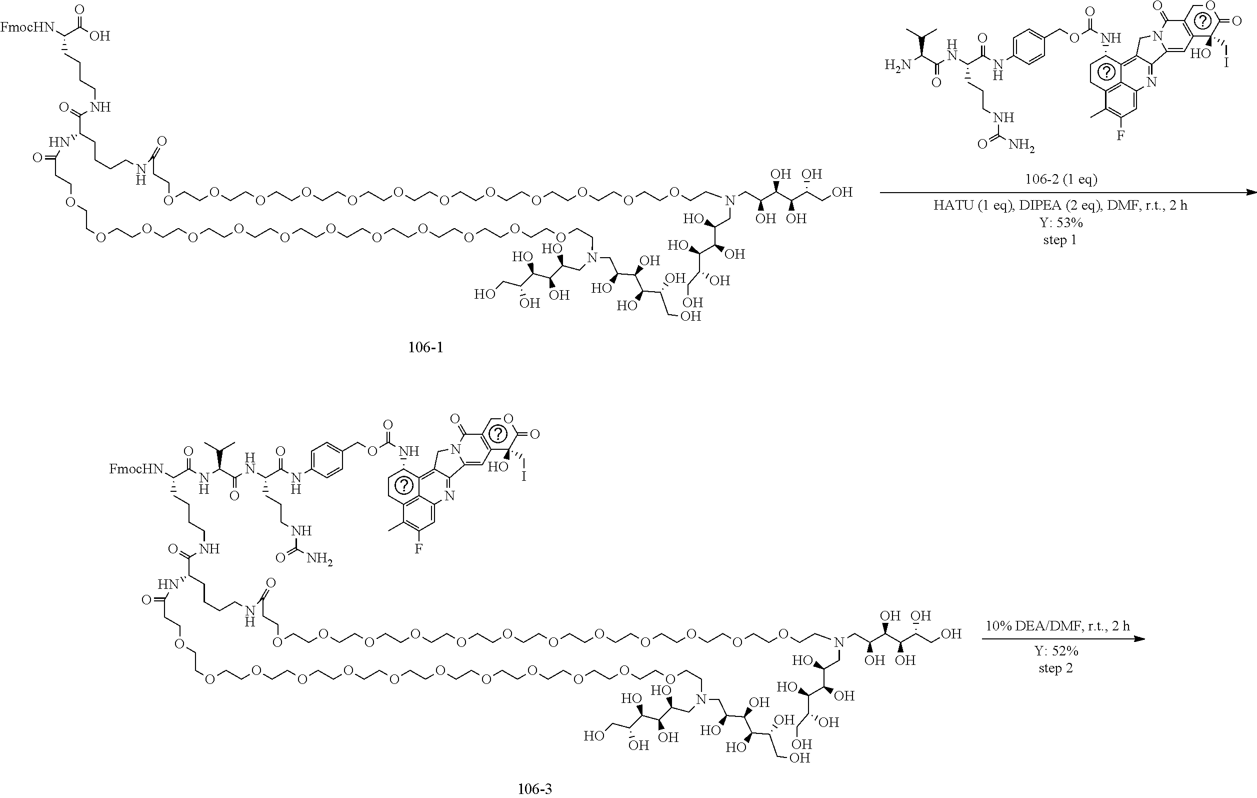

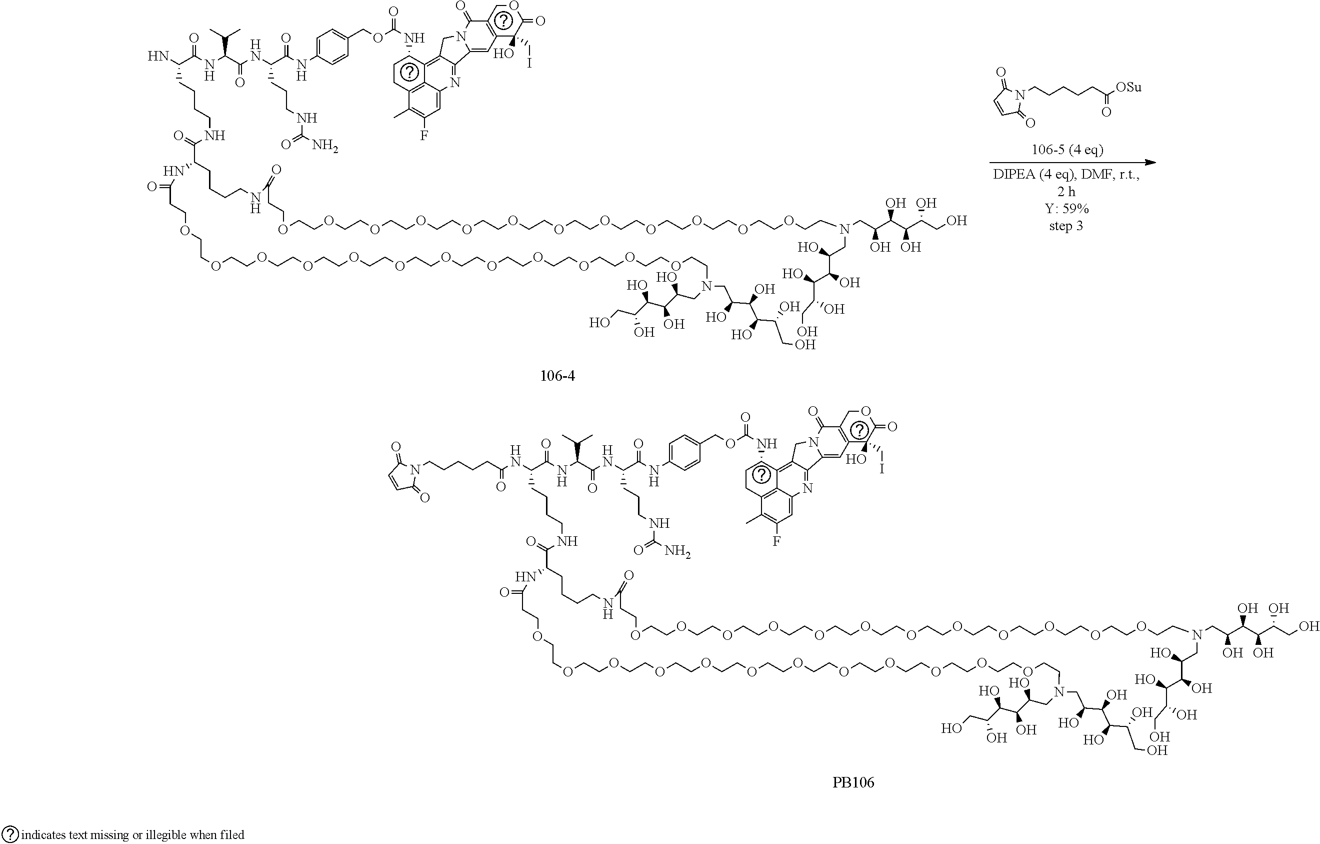

A Drug-Linker containing a PEG unit and a cleavable linker attached to exatecan (PB106) was prepared as follows:

### Step 1

A solution of compound 106-1 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (106-1, 200 mg, 0.085 mmol)), compound 106-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamate (106-2, 71.49 mg, 0.085 mmol)) and HATU (32.32 mg, 0.085 mmol), DIPEA (21.97 mg, 0.170 mmol) in DMF (2 mL) was stirred at room temperature for 2 h to completion. The reaction mixture was purified by reverse phase flash chromatography (0.01% TFA) to get the desired fractions, which were freeze-dried to yield compound 106-3 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamoyl}oxy)methyl]phenyl}carbamoyl)butyl]carbamoyl}-2-methylpropyl]carbamoyl}pentyl]carbamate (106-3, 144 mg, 0.045 mmol, 53.35%)) as a white solid. ESI m/z: 794.8 (M/4+H)⁺, 1059.3 (M/3+H)⁺.

### Step 2

A solution of compound 106-3 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (106-3, 144 mg, 0.045 mmol)) and diethylamine (0.2 mL, 0.180 mmol) in DMF (2 mL) was stirred at room temperature for 2 hour. Then the solution was purified by reverse phase flash chromatography (0.01% TFA) to yield compound 106-4 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}0.0\hat{ }{6,11}.0\hat{ }{20,24}]tetracosa-1,6(11),12,14,16,18,20(24)-heptaen-23-yl]carbamate (106-4, 70 mg, 0.024 mmol, 52.29%)) as a white solid. ESI m/z: 985.3 (M/3+H)⁺.

### Step 3

A solution of compound 106-4 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (106-4, 40 mg, 0.014 mmol), 2,5-dioxopyrrolidin-1-yl 6-(2,5-dioxo-2,5-dihydro-1H-pyrrol-1-yl)hexanoate (106-5, 17.26 mg, 0.056 mmol)) and DIPEA (7.24 mg, 0.056 mmol) in DMF (2 mL) was stirred at room temperature for 2 h to completion. Then the reaction mixture was purified by Prep-HPLC (0.01% TFA) to get the desired fractions, which were freeze-dried by LabConc. to yield PB106-6 ({4-[(2S)-2-[(2S)-2-[(2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}.0\hat{ }{4,13}.0\hat{ }{6,11}0.0\hat{ }{20,24}]tetracosa-1,6(11),12,14,16,18,20(24)- heptaen-23-yl]carbamate (PB106, 26 mg, 0.008 mmol, 59.02%)) as a pale yellow solid. ESI m/z: 787.5 (M/4+H)⁺, 1049.9 (M/3+H)⁺. Retention time 5.626 min (HPLC). ¹H NMR (400 MHz, DMSO-d6) δ 10.05 (s, 1H), 8.22-8.05 (m, 4H), 8.01-7.93 (m, 2H), 7.89-7.77 (m, 3H), 7.66 (d, J=8.0 Hz, 1H), 7.60 (d, J=8.2 Hz, 2H), 7.36 (d, J=8.4 Hz, 2H), 7.32 (s, 1H), 7.00 (s, 2H), 6.56 (s, 1H), 6.01 (d, J=14.8 Hz, 1H), 5.50-5.39 (m, 8H), 5.33-5.24 (m, 3H), 5.08 (s, 2H), 4.88-4.67 (m, 4H), 4.67-4.38 (m, 12H), 4.38-4.13 (m, 4H), 4.00-3.94 (m, 4H), 3.83-3.77 (m, 4H), 3.72-3.63 (m, 4H), 3.63-3.56 (m, 18H), 3.55-3.46 (m, 88H), 3.38-3.14 (m, 9H), 3.14-2.91 (m, 10H), 2.38-2.30 (m, 5H), 2.28 (t, J=6.8 Hz, 2H), 2.23-2.08 (m, 5H), 2.02-1.83 (m, 4H), 1.72-1.53 (m, 5H), 1.46-1.28 (m, 13H), 1.28-1.12 (m, 7H), 0.90-0.80 (m, 9H) ppm. Two protons of carboxyl group from TFA were also revealed. Drug linker PB106 can be used to make a conjugate such as PA106.

### Example 38: Preparation of the Drug-Linker (PB107) Containing a PEG Unit and a Cleavable Linker Attached to Exatecan

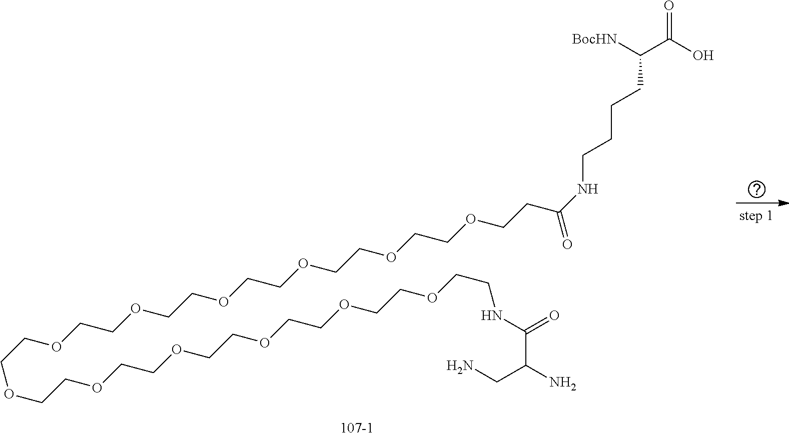 [SMILES: CC(C)(C)OC(=O)N[C@@H](CCCCNC(=O)CCOCCOCCOCCOCCOCCOCCOCCOCCOCCOCCOCCOCCNC(=O)C(N)CN)C(=O)O]

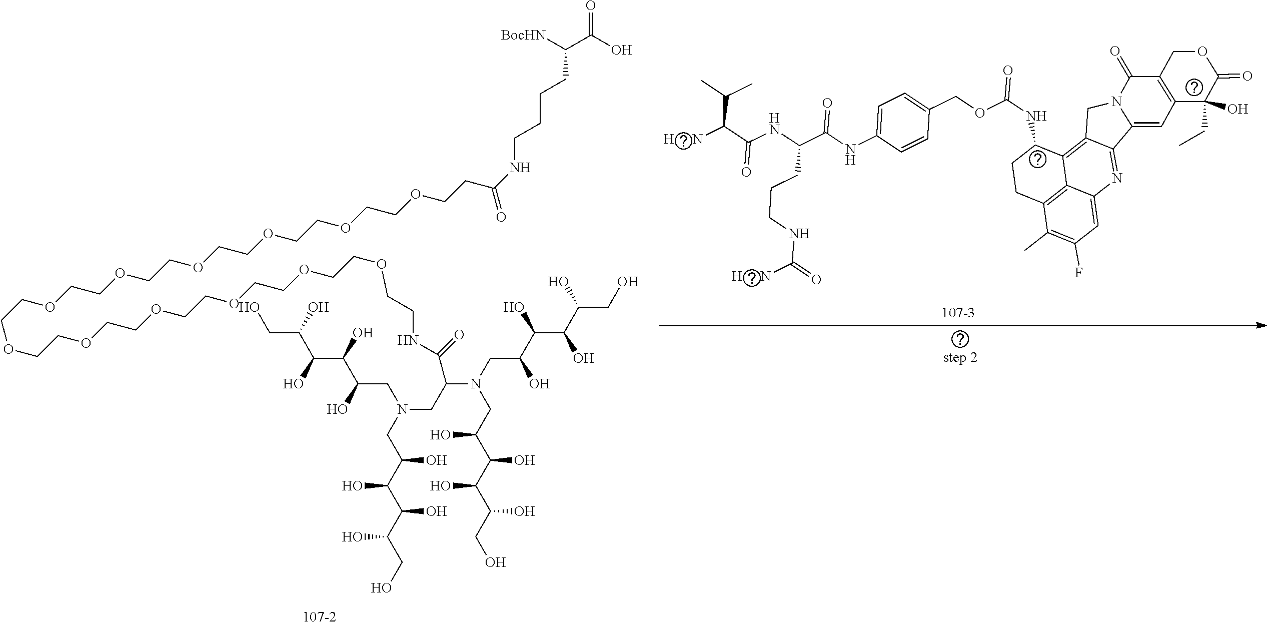

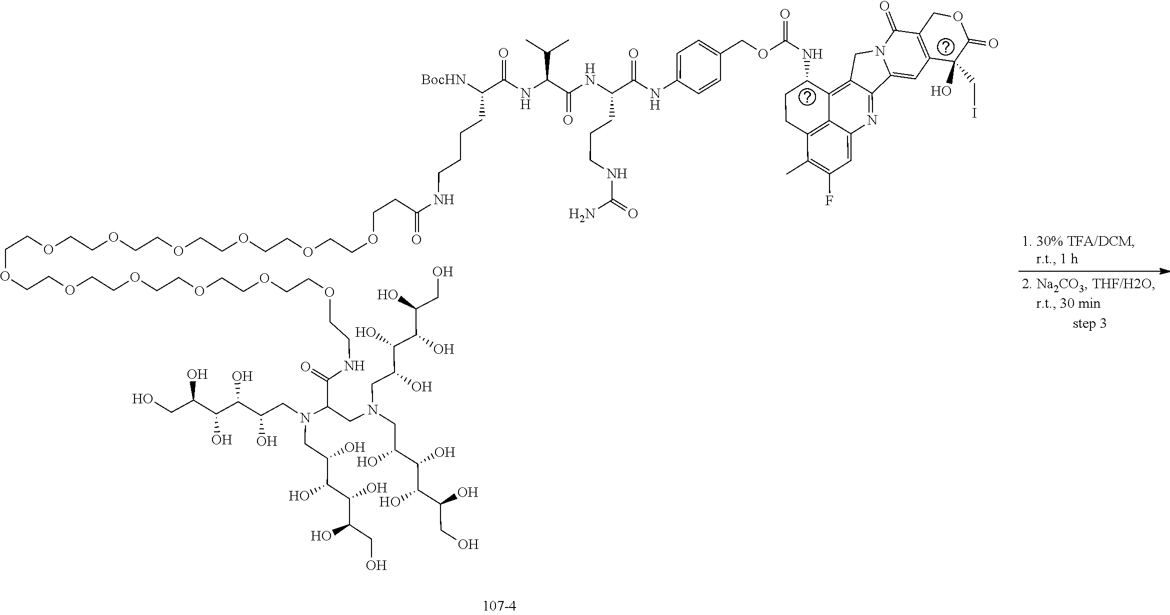 [SMILES: Cc1c(F)cc2nc3c(c4c2c1CC[C@@H]4NC(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)N(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc1)Cn1c-3cc2c(c1=O)COC(=O)[C@]2(O)CI]

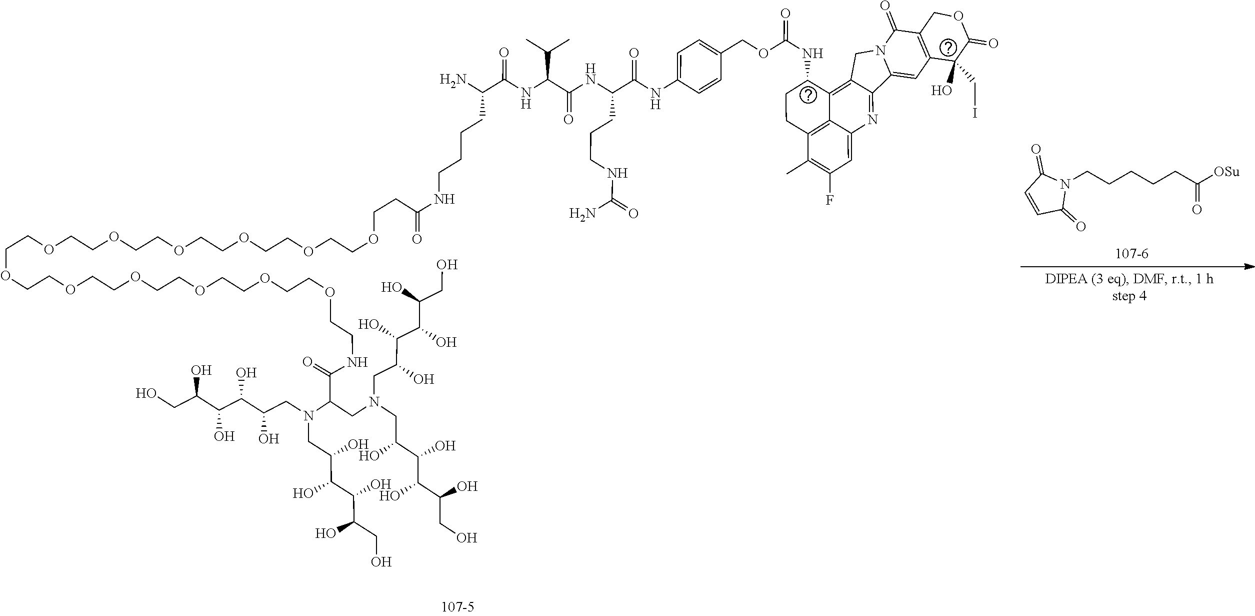

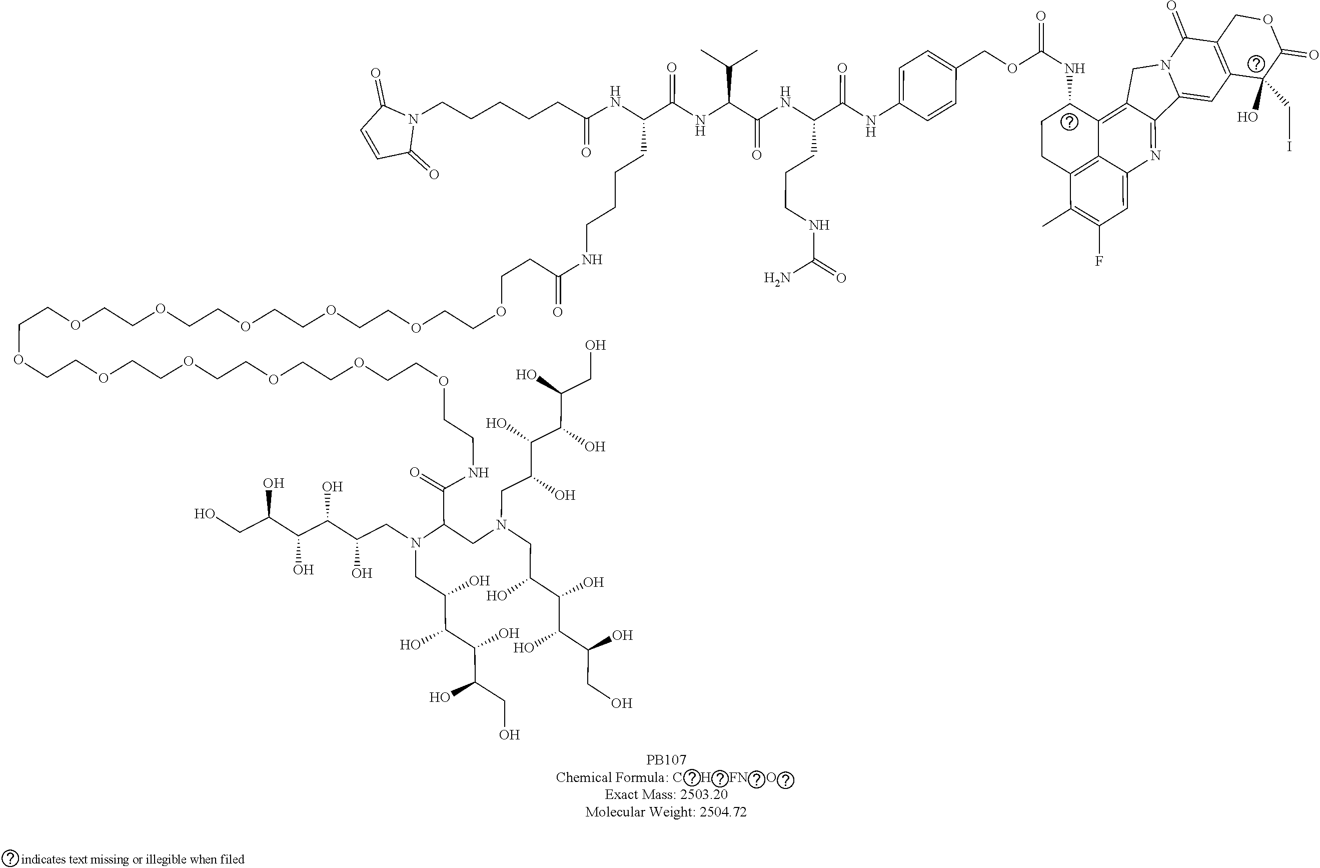

A Drug-Linker containing a PEG unit and a cleavable linker attached to exatecan (PB107) was prepared as follows:

### Step 1

A solution of compound 107-1 (0.5 g, 0.536 mmol) and D-glucose (0.77 g, 4.291 mmol) in anhydrous methanol (50 mL) was heated at 50° C. for 30 min, then NaCNBH₃ (0.27 g, 4.291 mmol) was added. The resulting solution was stirred for another 4 days at 70° C. until LCMS indicated all starting amine was consumed and the mass of desired product was detected. Then the reaction solution was concentrated and purified by reverse phase liquid chromatography to yield compound 107-2 (0.3 g, 0.189 mmol, 35.29%) as a white solid, purity=85%-90%.

### Step 2

A solution of compound 107-3 (0.16 g, 0.189 mmol), compound 107-2 (0.3 g, 0.189 mmol) and HATU (72 mg, 0.189 mmol) in anhydrous DMF (4 mL) was stirred at room temperature for 5 min, then DIPEA (74 mg, 0.567 mmol) was added. The resulting solution was stirred for another 1 hr at r.t. until LCMS indicated complete reaction. The reaction solution was purified directly by reverse phase liquid chromatography to yield compound 107-4 (140 mg, 0.058 mmol, 30.50%) as a white solid, purity=90%-95%.

### Step 3

A solution of compound 107-4 (140 mg, 0.058 mmol) and TFA (1 mL) in anhydrous DCM (4 mL) was stirred at room temperature for 1 hr until LCMS indicated all starting amine was consumed and the desired product (m/z=1156=2311/2+H) along with sugar-esterificated product (TFA was condensed with hydroxy group in sugar unit, mono-ester with m/z=(2311+96)/3+H=803) were formed. The completed reaction solution was condensed to dryness and then redissolved in THE (4 mL) and Water (2 mL), treated with saturated aqueous sodium carbonate solution to adjust pH to 8-9. The resulting solution was stirred at room temperature for 30 min to achieve complete hydrolysis. The solution was then treated with diluted HCl to adjust pH to 2-3 and condensed, the residue was purified by reverse phase liquid chromatography to yield compound 107-5 (113 mg, 0.049 mmol, 84.32%) as a white solid. purity=90%-95%.

### Step 4

A solution of compound 107-5 (113 mg, 0.049 mmol) and DIPEA (19 mg, 0.147 mmol) in anhydrous DMF (2 mL) was stirred at room temperature for 5 min, then a solution of compound 107-6 (23 mg, 0.074 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB107 (12 mg, 0.005 mmol, 9.84%) as a white solid. LCMS, m/z=835.93 (M/3+H)⁺. Drug linker PB107 can be used to make a conjugate such as PA107.

### Example 39: Preparation of the Drug-Linker (PB108) Containing a PEG Unit and a Cleavable Linker Attached to Exatecan

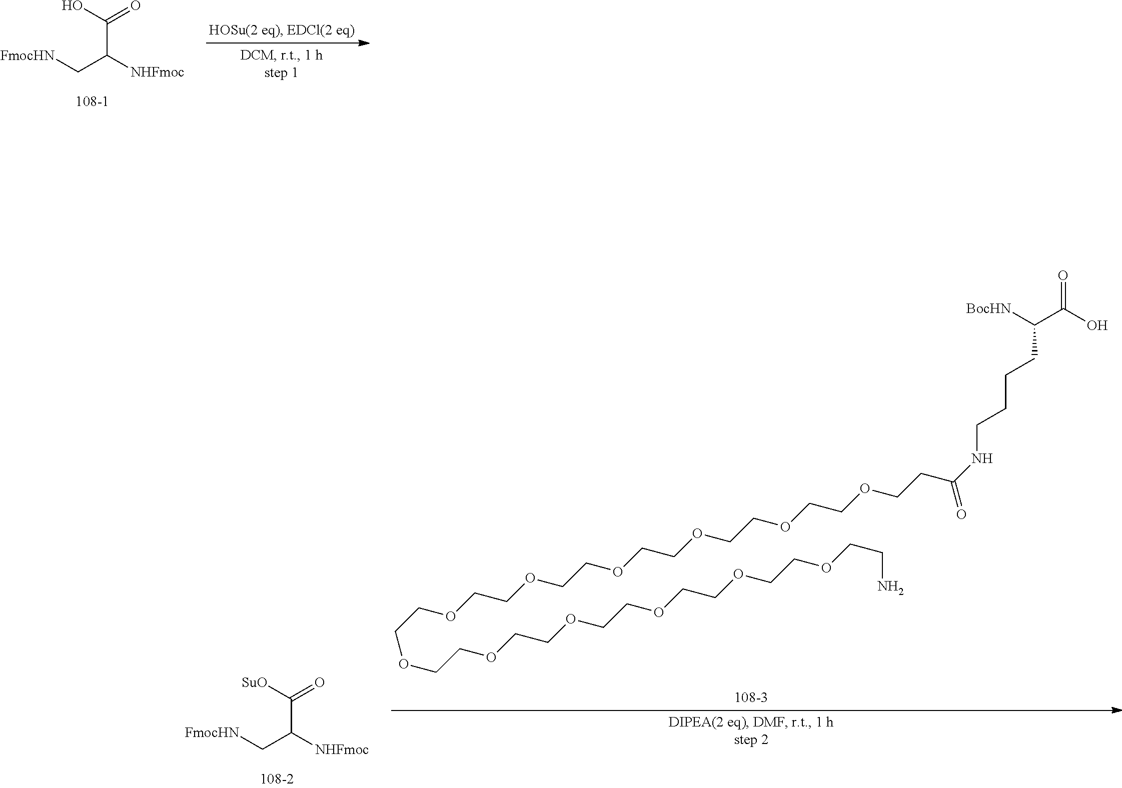

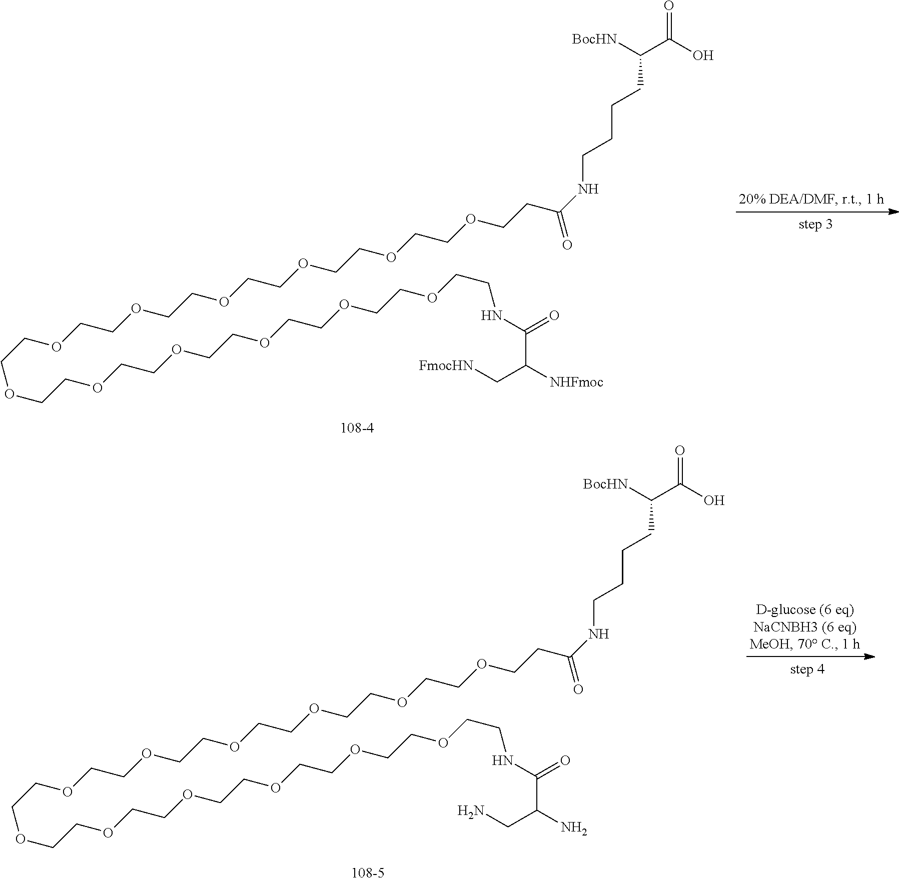

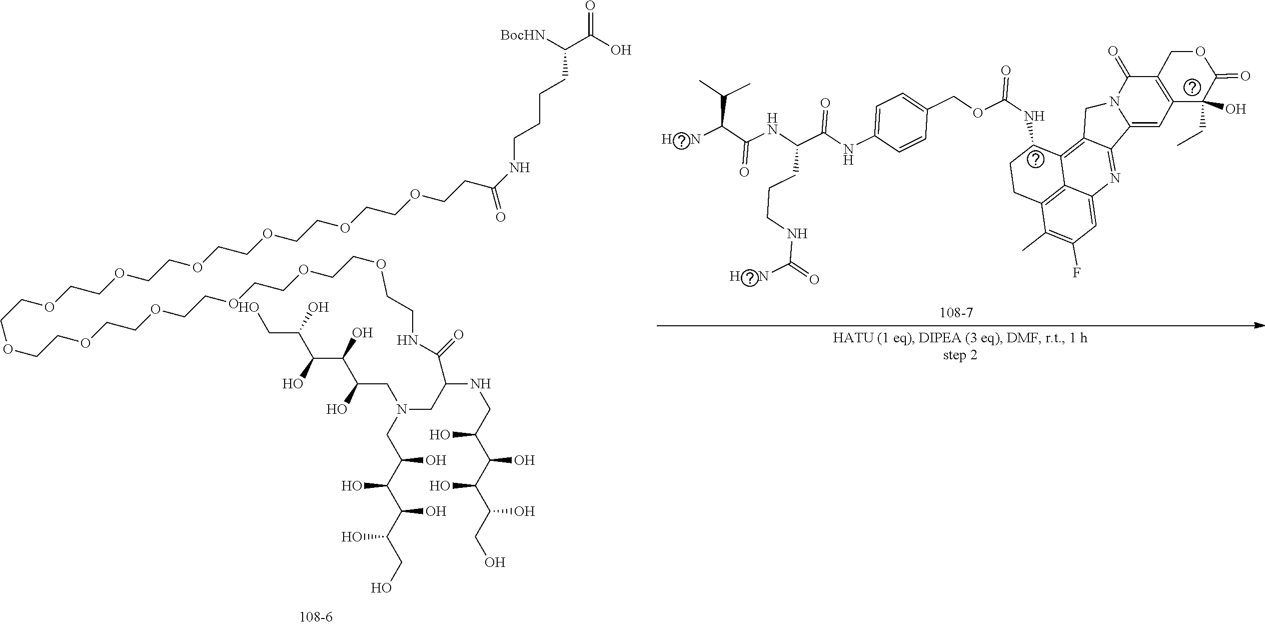

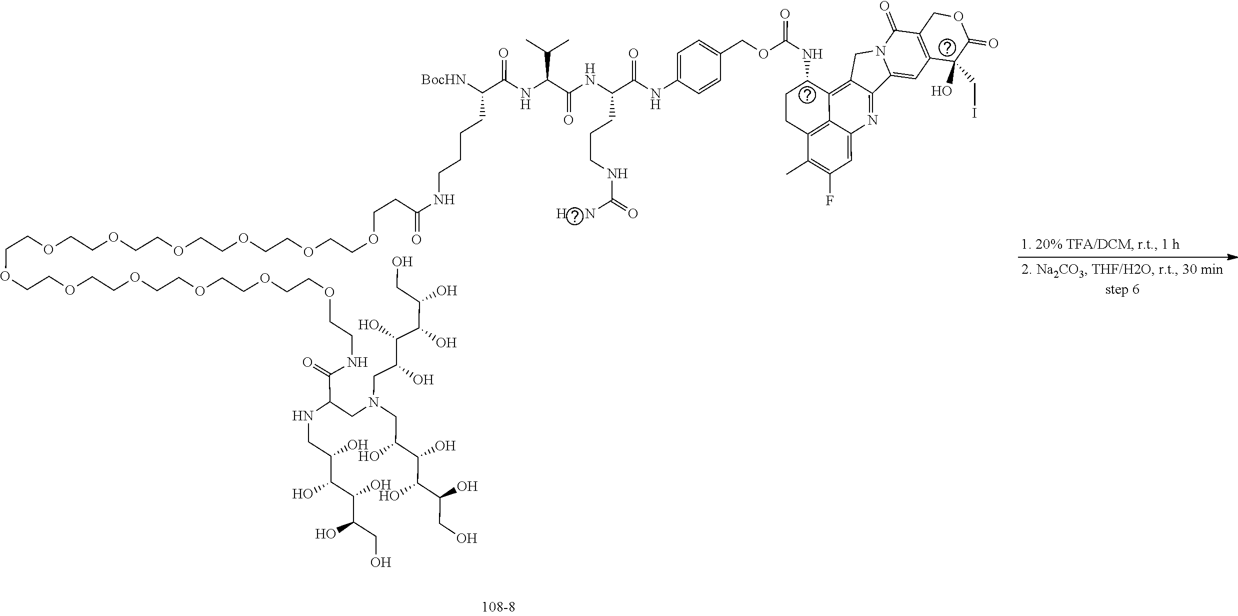

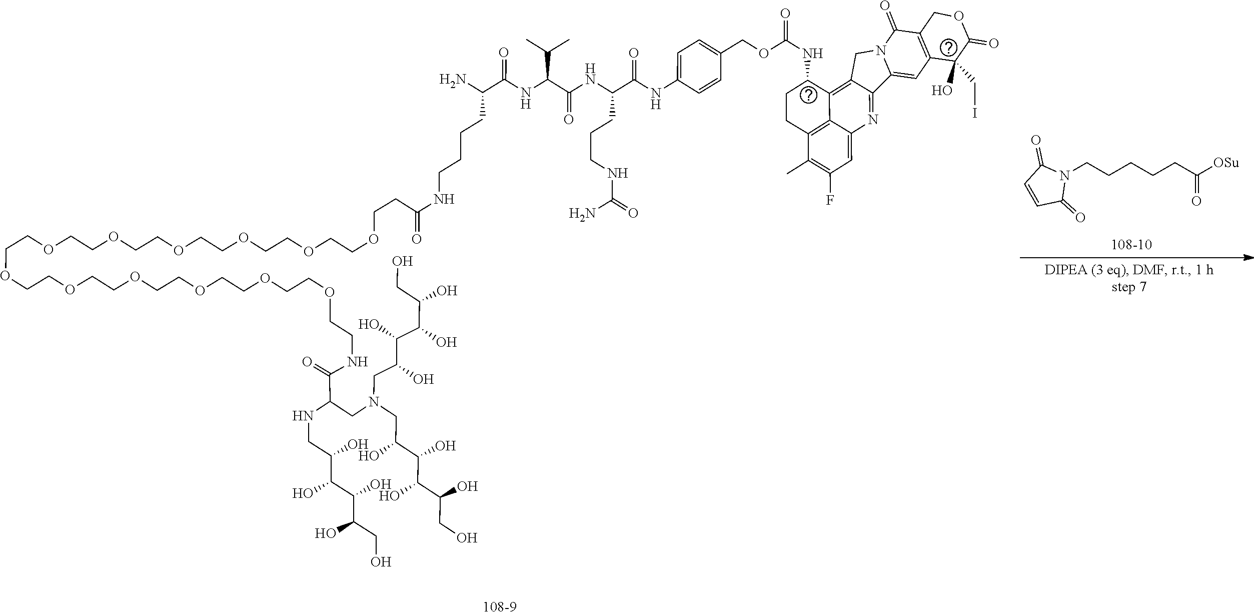

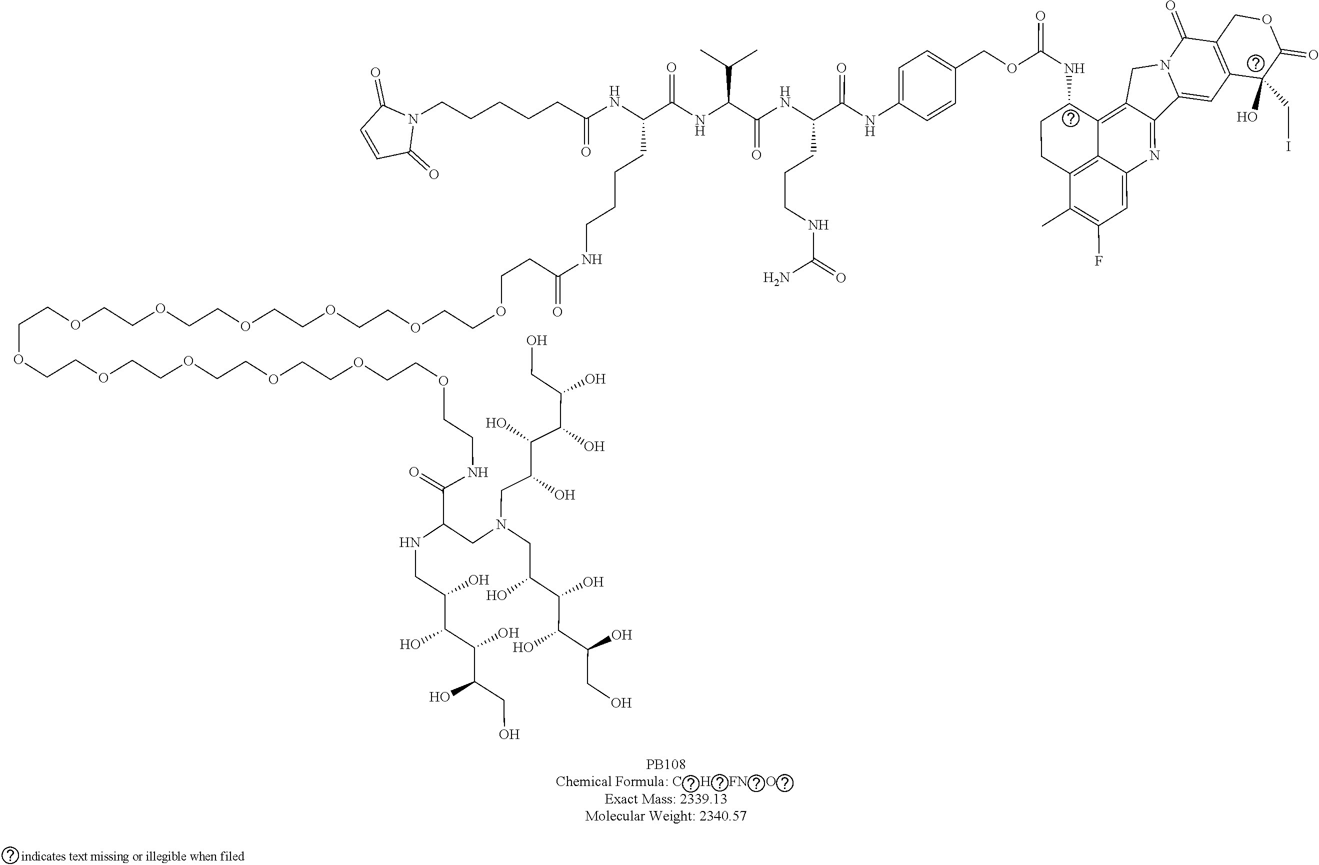

A Drug-Linker containing a PEG unit and a cleavable linker attached to exatecan (PB107) was prepared as follows:

### Step 1

A solution of compound 108-1 (2 g, 3.646 mmol) and HOSu (0.84 g, 7.291 mmol) in anhydrous DCM (30 mL) was stirred at room temperature, then EDCI (1.4 g, 7.291 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (40 mL*2). The combined organic layer was dried over sodium sulfate and filtered, concentrated to dryness to yield compound 108-2 (1.8 g, 2.788 mmol, 76.60%) as a white solid, which was used as such in the next step. purity=85%-90%.

### Step 2

A solution of compound 108-2 (0.76 g, 1.182 mmol) and compound 108-3 (1 g, 1.182 mmol) in anhydrous DMF (5 mL) was stirred at room temperature, then DIPEA (0.3 g, 2.364 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 108-4 (1.4 g, 1.017 mmol, 85.89%) as a white solid. purity=90%-95%.

### Step 3

A solution of compound 108-4 (1.4 g, 1.017 mmol) and DEA (5 mL) in anhydrous DMF (20 mL) was stirred at room temperature for 1 hr until LCMS indicated all starting amine was consumed and the desired product was detected. Then the solution was concentrated to dryness to yield compound 108-5 (0.9 g, 0.966 mmol, 94.74%) as yellow oil, which was used as such in the next step. purity=90%-95%.

### Step 4

A solution of compound 108-5 (0.9 g, 0.966 mmol) and D-Glucose (1.04 g, 5.793 mmol) in anhydrous methanol (50 mL) was heated at 50° C. for 30 min, then NaCNBH₃ (0.36 g, 5.793 mmol) was added. The resulting solution was stirred for another 1 hr at 70° C. until LCMS indicated all starting amine was consumed and the mass of desired product was detected. Then the reaction solution was concentrated and purified by reverse phase liquid chromatography to yield compound 108-6 (0.7 g, 0.492 mmol, 50.72%) as a white solid. purity=85%-90%.

### Step 5

A solution of compound 108-7 (0.21 g, 0.250 mmol), compound 108-6 (0.36 g, 0.250 mmol) and HATU (93 mg, 0.250 mmol) in anhydrous DMF (4 mL) was stirred at room temperature for 5 min, then DIPEA (95 mg, 0.750 mmol) was added. The resulting solution was stirred for another 1 hr at r.t. until LCMS indicated complete reaction. The reaction solution was purified directly by reverse phase liquid chromatography to yield compound 108-8 (0.21 g, 0.093 mmol, 37.50%) as a white solid. purity=90%-95%.

### Step 6

A solution of compound 108-8 (0.21 g, 0.093 mmol) and TFA (1 mL) in anhydrous DCM (4 mL) was stirred at room temperature for 1 hr until LCMS indicated all starting amine was consumed and the desired product (m/z=1074=2147/2+H) along with a sugar-esterificated product (TFA was condensed with hydroxy group in sugar unit, mono-ester with m/z=(2147+96)/2+H=1122) were formed. The completed reaction solution was condensed to dryness and then redissolved in THE (4 mL) and Water (2 mL), treated with saturated aqueous sodium carbonate solution to adjust the pH to 8-9. The resulting solution was stirred at room temperature for 30 min to achieve complete hydrolysis. The solution was then treated with diluted HCl to adjust the pH to 2-3 and condensed, the residue was purified by reverse phase liquid chromatography to yield compound 108-9 (160 mg, 0.075 mmol, 80.65%) as a white solid. purity=90%-95%.

### Step 7

A solution of compound 108-9 (160 mg, 0.075 mmol) and DIPEA (28.8 mg, 0.224 mmol) in anhydrous DMF (2 mL) was stirred at room temperature for 5 min, then a solution of compound 108-10 (34.4 mg, 0.113 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB108 (15 mg, 0.006 mmol, 8.62%) as a white solid. LCMS, m/z=1171.24 (M/2+H)⁺. Drug linker PB108 can be used to make a conjugate such as PA108.

### Example 40: Preparation of the Drug-Linker (PB109) Containing a PEG Unit and a Cleavable Linker Attached to MMAE

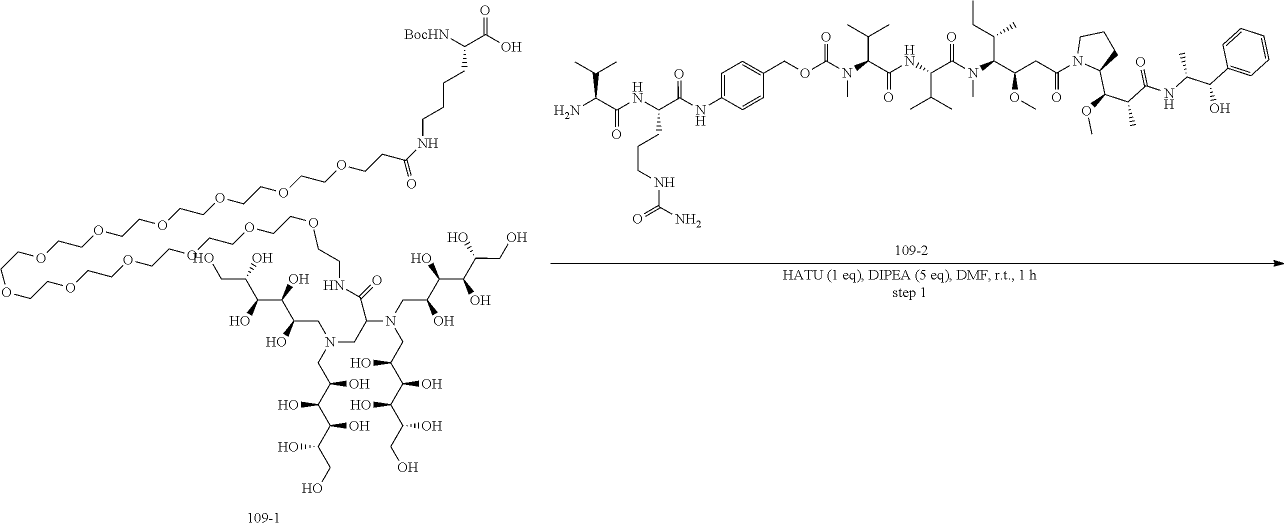 [SMILES: CC(C)(C)OC(=O)N[C@@H](CCCCNC(=O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)N(C[C@H](O)[C@@H](O)[C@H](O)[C@H](O)CO)C[C@H](O)[C@@H](O)[C@H](O)[C@H](O)CO)C(=O)O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

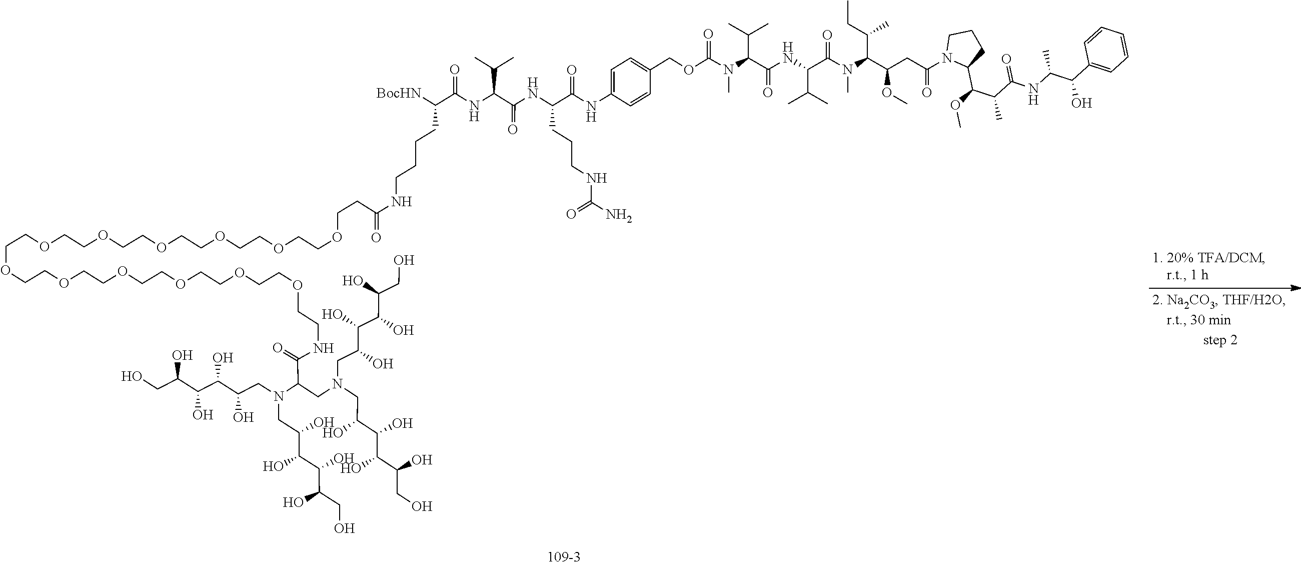

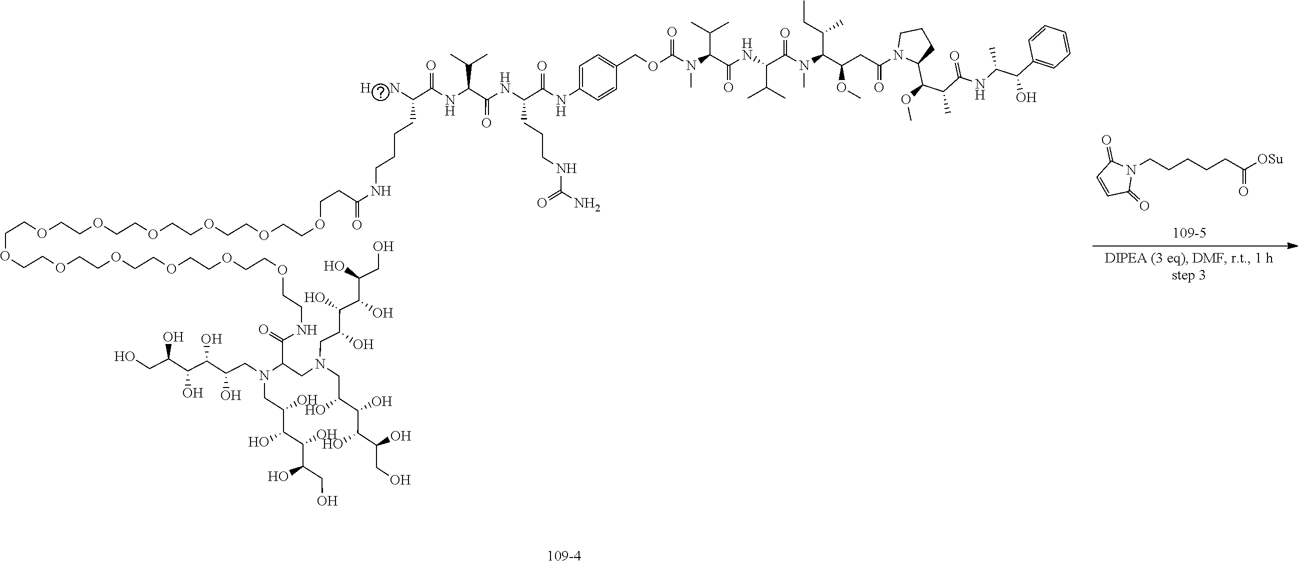

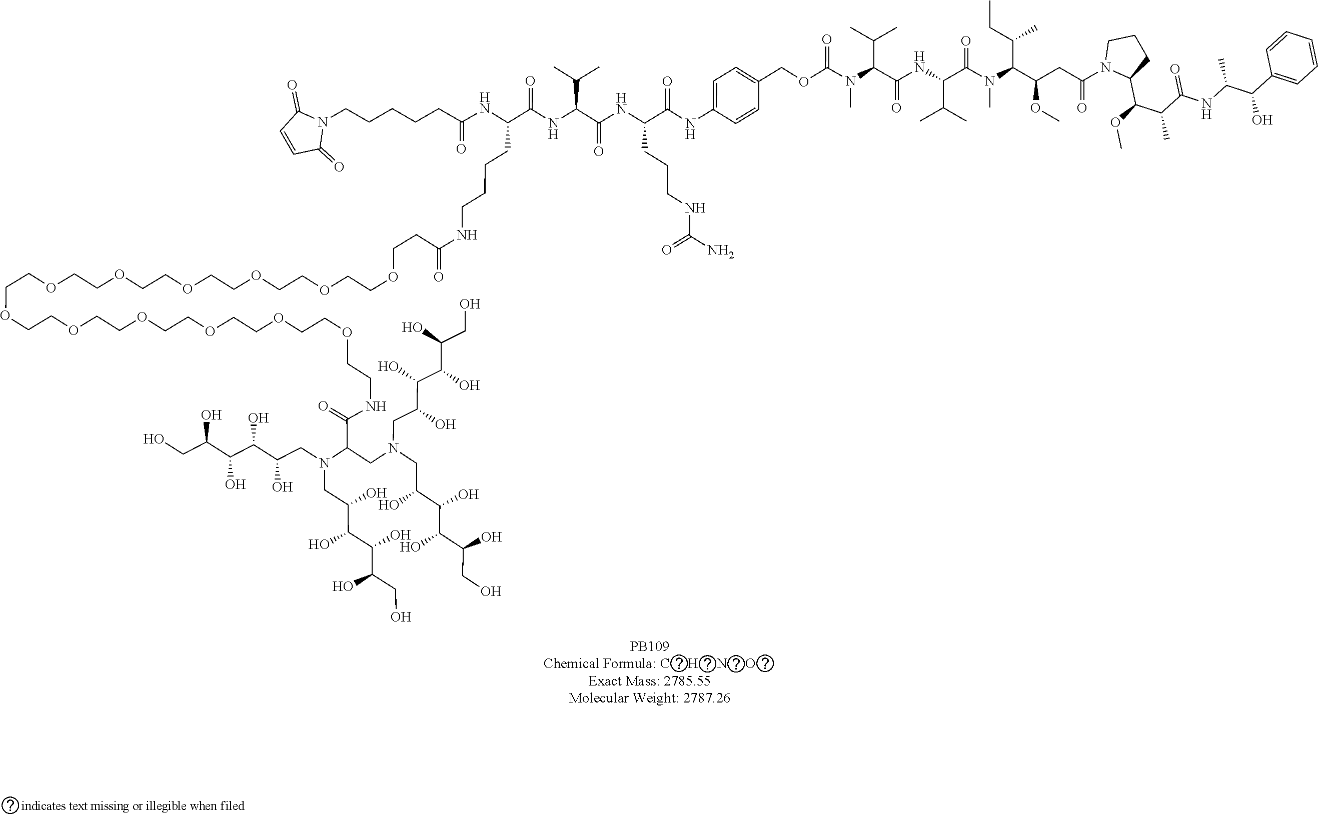

A Drug-Linker containing a PEG unit and a cleavable linker attached to MMAE (PB109) was prepared as follows:

### Step 1

A solution of compound 109-1 (170 mg, 0.107 mmol), compound 109-2 (120 mg, 0.107 mmol) and HATU (41 mg, 0.107 mmol) in anhydrous DMF (4 mL) was stirred at room temperature for 5 min, then DIPEA (41.4 mg, 0.321 mmol) was added. The resulting solution was stirred for another 1 hr at r.t. until LCMS indicated complete reaction. The reaction solution was purified directly by reverse phase liquid chromatography to yield compound 109-3 (103 mg, 0.038 mmol, 35.76%) as a white solid. purity=90%-95%.

### Step 2

A solution of compound 109-3 (103 mg, 0.038 mmol) and TFA (1 mL) in anhydrous DCM (4 mL) was stirred at room temperature for 1 hr until LCMS indicated all starting amine was consumed and the desired product (m/z=865=2594/3+H) along with sugar-esterificated product (TFA was condensed with hydroxy group in sugar unit, mono-ester with m/z=(2594+96)/3+H=897) were formed. The completed reaction solution was condensed to dryness and then redissolved in THE (4 mL) and water (2 mL), and treated with saturated aqueous sodium carbonate solution to adjust the pH to 8-9. The resulting solution was stirred at room temperature for 30 min to achieve complete hydrolysis. The solution was then treated with diluted HCl to adjust the pH to 6-7 and condensed; the residue was purified by reverse phase liquid chromatography to yield compound 109-4 (45 mg, 0.017 mmol, 45.45%) as a white solid. purity=90%-95%.

### Step 3

A solution of compound 109-4 (45 mg, 0.017 mmol) and DIPEA (6.7 mg, 0.052 mmol) in anhydrous DMF (2 mL) was stirred at room temperature for 5 min, then a solution of 109-5 (8.0 mg, 0.026 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB109 (6 mg, 0.002 mmol, 12.41%) as a white solid. LCMS, m/z=930.16 (M/3+H)⁺. Drug linker PB109 can be used to make a conjugate such as PA109.

### Example 41: Preparation of the Drug-Linker (PB110) Containing a PEG Unit and a Cleavable Linker Attached to MMAE

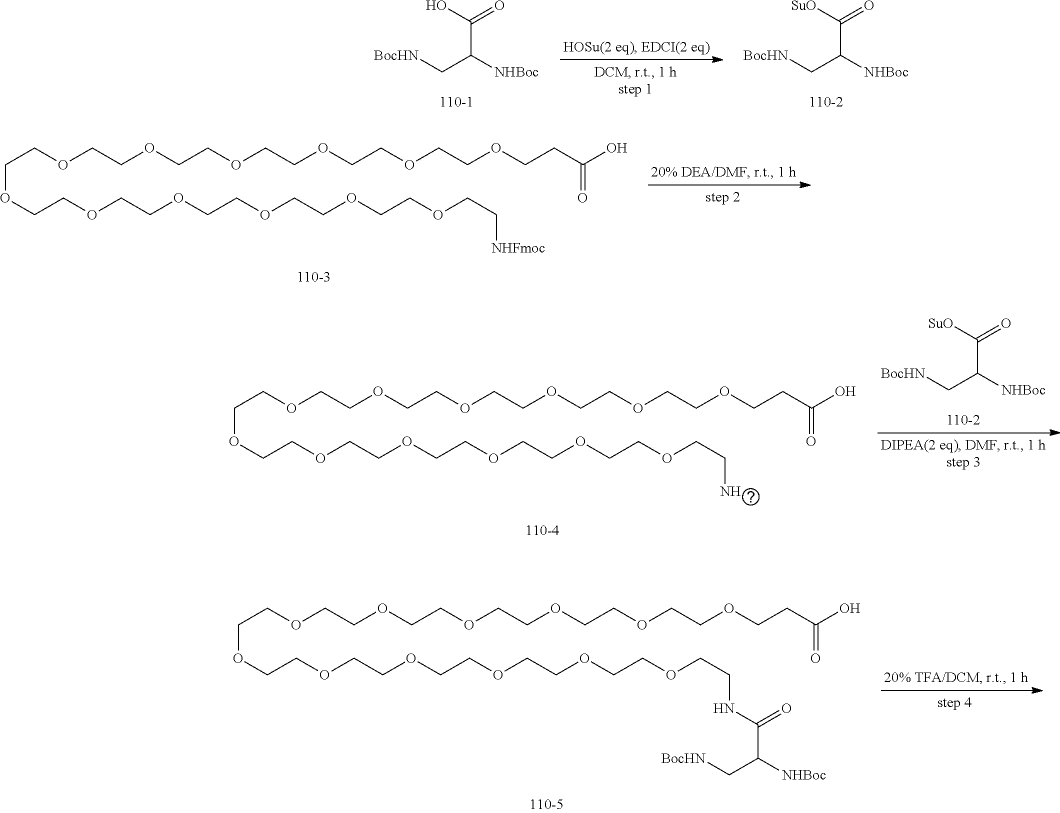

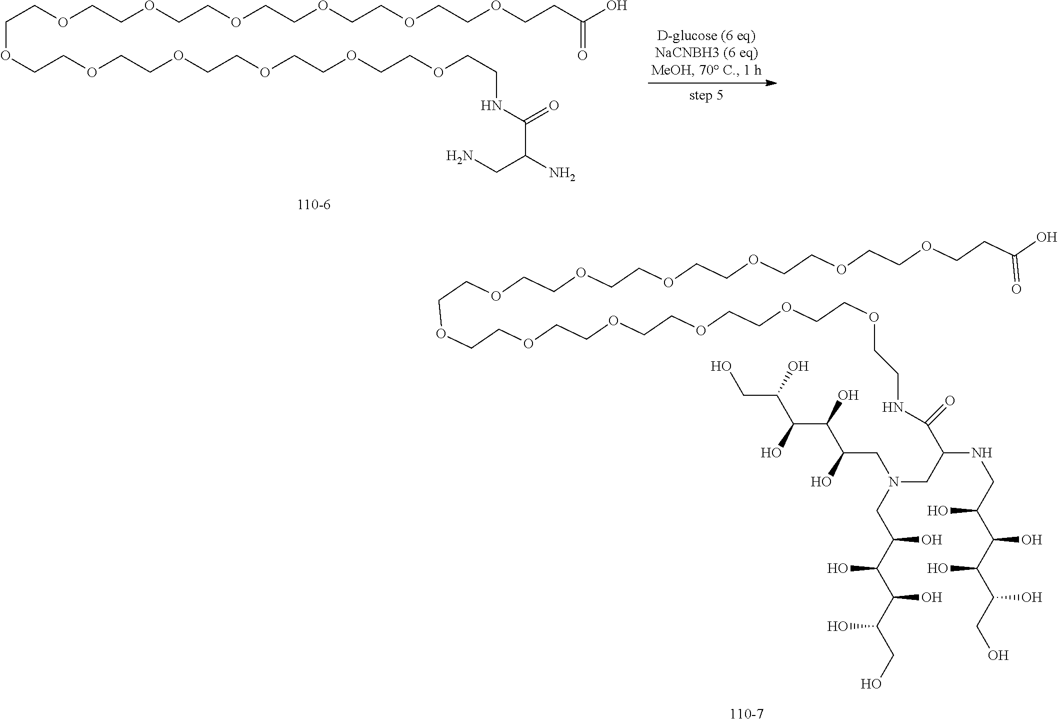 [SMILES: NCC(N)C(=O)NCCOCCOCCOCCOCCOCCOCCOCCOCCOCCOCCOCCOCCC(=O)O ; O=C(O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)NC[C@H](O)[C@@H](O)[C@H](O)[C@H](O)CO]

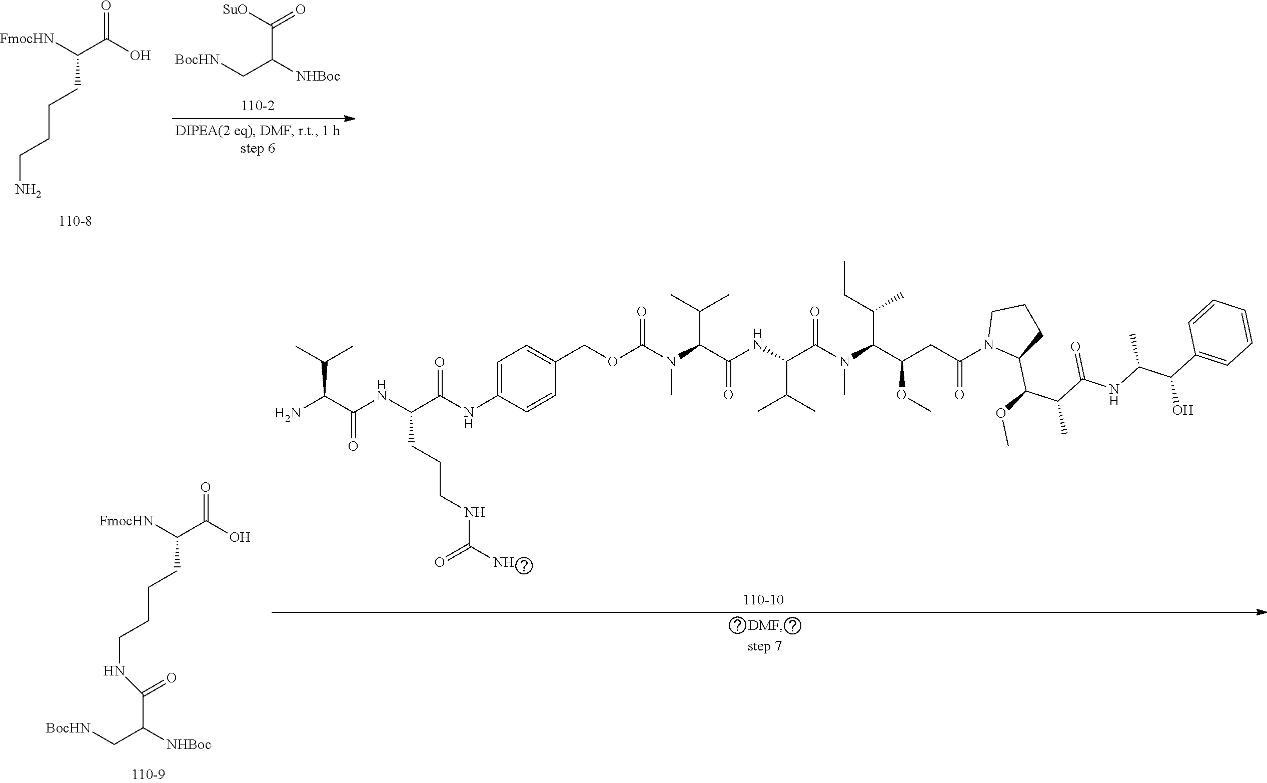

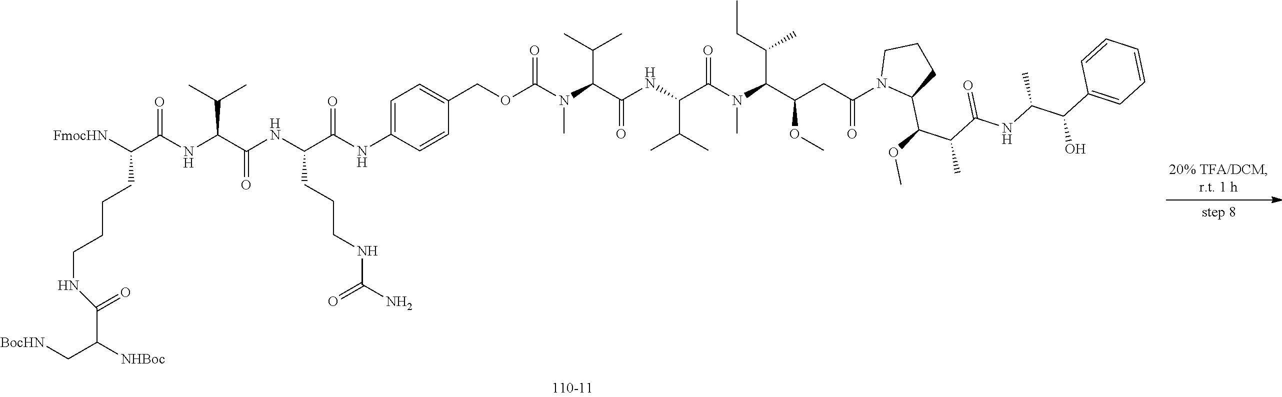

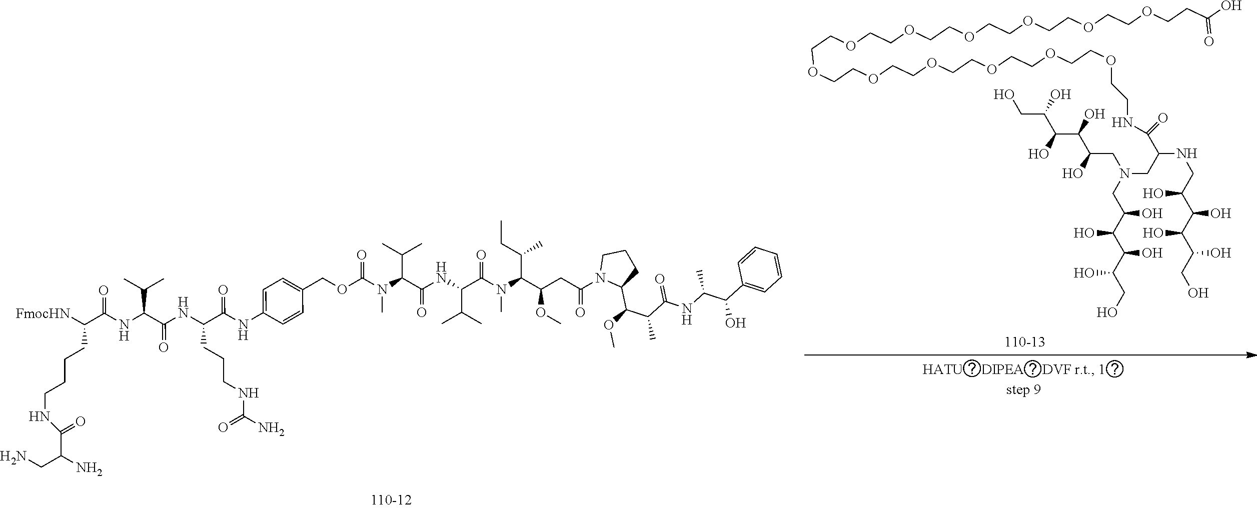

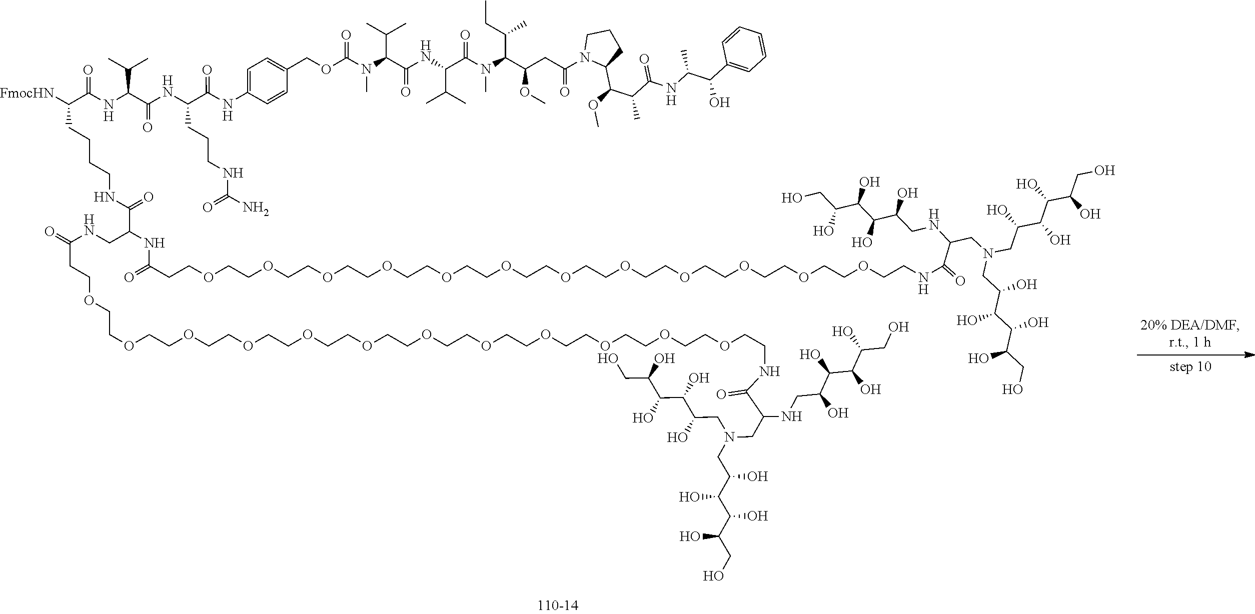

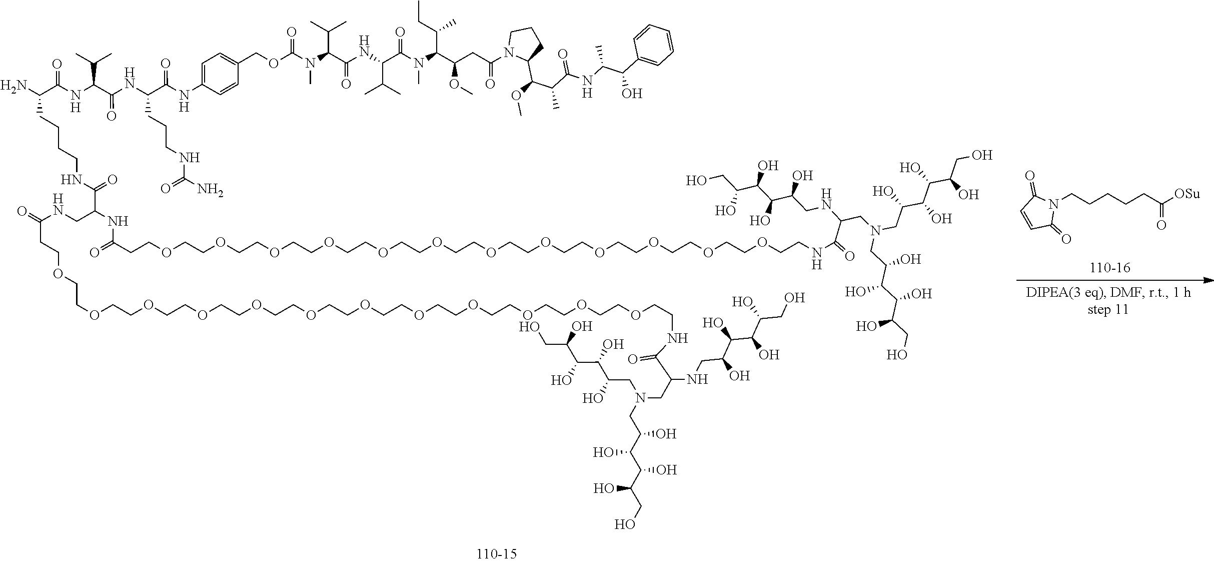

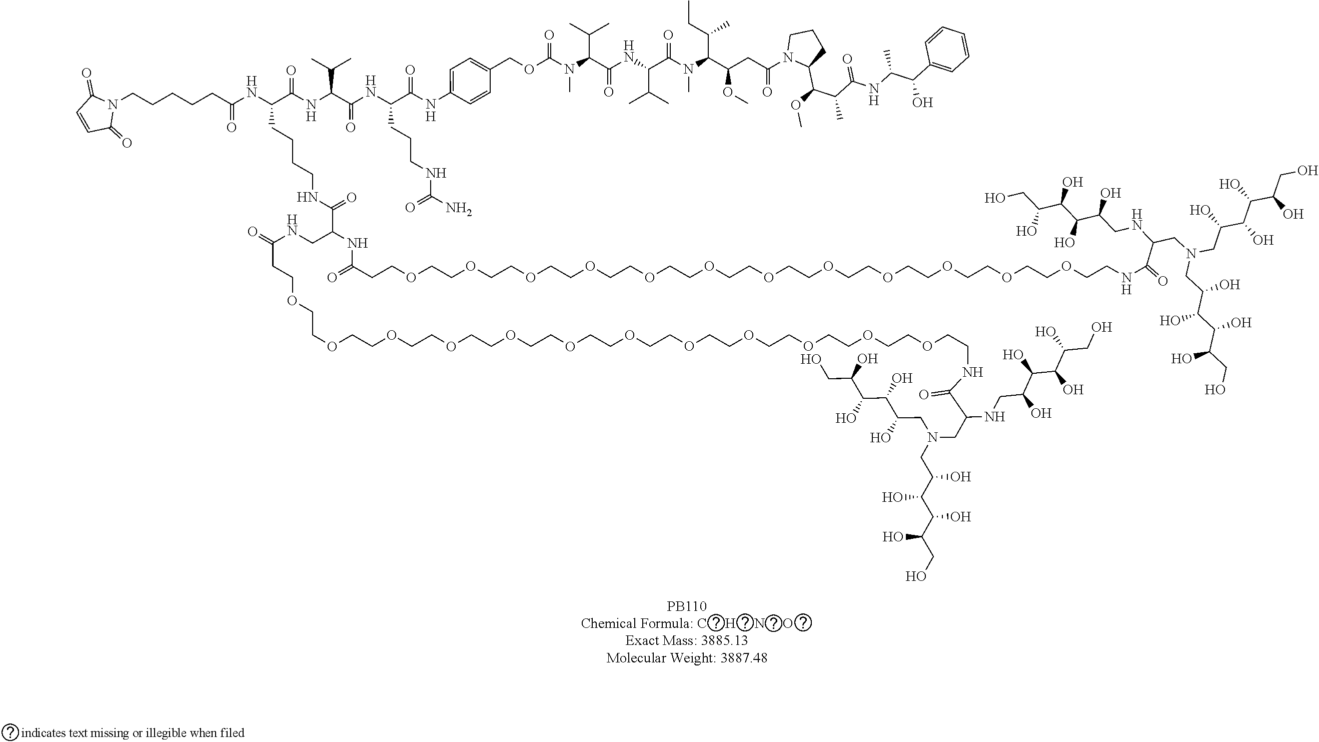

A Drug-Linker containing a PEG unit and a cleavable linker attached to MMAE (PB110 or LD110) was prepared as follows:

### Step 1

A solution of compound 110-1 (5 g, 8.671 mmol) and HOSu (2.0 g, 17.342 mmol) in anhydrous DCM (150 mL) was stirred at room temperature, then EDCI (3.3 g, 17.342 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (100 mL*2). The combined organic layer was dried over sodium sulfate and filtered, concentrated to dryness to yield compound 110-2 (4.7 g, 6.976 mmol, 80.48%) as a white solid, which was used as such in the next step. purity=85%-90%.

### Step 2

A solution of compound 110-3 (5 g, 5.953 mmol) and DEA (5 mL) in anhydrous DMF (20 mL) was stirred at room temperature for 1 hr until LCMS indicated all starting amine was consumed and the desired product was detected. Then the solution was concentrated to dryness to yield compound 110-4 (3.4 g, 5.504 mmol, 92.39%) as colorless oil, which was used as such in the next step. purity=90%-95%.

### Step 3

A solution of compound 110-2 (1 g, 2.491 mmol) and compound 110-4 (1.5 g, 2.491 mmol) in anhydrous DMF (10 mL) was stirred at room temperature, then DIPEA (0.64 g, 4.982 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 110-5 (1.3 g, 1.438 mmol, 57.78%) as a colorless oil. purity=90%-95%.

### Step 4

A solution of compound 110-5 (1.3 g, 1.438 mmol) and TFA (4 mL) in anhydrous DCM (16 mL) was stirred at room temperature for 1 hr until LCMS indicated all starting amine was consumed and the desired product was detected. Then the solution was concentrated to dryness to yield compound 110-6 (910 mg, 1.293 mmol, 89.92%) as yellow oil, which was used as such in the next step. purity=85%-90%.

### Step 5

A solution of compound 110-6 (910 mg, 1.293 mmol) and D-glucose (1.4 g, 7.771 mmol) in anhydrous methanol (40 mL) was heated at 50° C. for 30 min, then NaCNBH₃ (488 mg, 7.766 mmol) was added. The resulting solution was stirred for another 1 hr at 70° C. until LCMS indicated all starting amine was consumed and the mass of desired product was detected. Then the reaction solution was concentrated and purified by reverse phase liquid chromatography to yield compound 110-7 (320 mg, 0.268 mmol, 20.70%) as a white solid. purity=85%-90%.

### Step 6

A solution of compound 110-2 (1 g, 2.491 mmol) and compound 110-8 (0.92 g, 2.491 mmol) in anhydrous DMF (10 mL) was stirred at room temperature, then DIPEA (0.64 g, 4.982 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 110-9 (1.4 g, 2.138 mmol, 85.89%) as a white solid. purity=90%-95%.

### Step 7

A solution of compound 110-9 (0.58 g, 0.886 mmol), compound 110-10 (1 g, 0.890 mmol) and HATU (0.34 g, 0.894 mmol) in anhydrous DMF (10 mL) was stirred at room temperature for 5 min, then DIPEA (0.35 g, 2.708 mmol) was added. The resulting solution was stirred for another 1 hr at r.t. until LCMS indicated complete reaction. The reaction solution was purified directly by reverse phase liquid chromatography to yield compound 110-11 (240 mg, 0.136 mmol, 15.29%) as a white solid. purity=90%-95%.

### Step 8

A solution of compound 110-11 (240 mg, 0.136 mmol) and TFA (1 mL) in anhydrous DCM (4 mL) was stirred at room temperature for 1 hr until LCMS indicated all starting amine was consumed and the desired product was detected. Then the solution was concentrated to dryness to yield compound 110-12 (195 mg, 0.125 mmol, 91.68%) as a yellow oil, which was used as such in the next step. purity=85%-90%.

### Step 9

A solution of compound 110-12 (195 mg, 0.125 mmol), compound 110-13 (299 mg, 0.250 mmol) and HATU (95 mg, 0.250 mmol) in anhydrous DMF (5 mL) was stirred at room temperature for 5 min, then DIPEA (97 mg, 0.751 mmol) was added. The resulting solution was stirred for another 1 hr at r.t. until LCMS indicated complete reaction. The reaction solution was purified directly by reverse phase liquid chromatography to yield compound 110-14 (210 mg, 0.053 mmol, 42.89%) as a white solid. purity=90%-95%.

### Step 10

A solution of compound 110-14 (210 mg, 0.053 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 110-15 (175 mg, 0.047 mmol, 88.38%) as colorless oil, which was used as such in the next step. purity=90%-95%.

### Step 11

A solution of compound 110-15 (175 mg, 0.047 mmol) and DIPEA (12.2 mg, 0.094 mmol) in anhydrous DMF (2 mL) was stirred at room temperature for 5 min, then a solution of compound 110-16 (21.9 mg, 0.071 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB110 (73 mg, 0.019 mmol, 39.63%) as a white solid. LCMS, m/z=972.82 (M/4+H)⁺. Drug linker PB110 can be used to make a conjugate such as PA110.

### Example 42: Preparation of the Drug-Linker (PB111) Containing a PEG Unit and a Cleavable Linker Attached to Exatecan

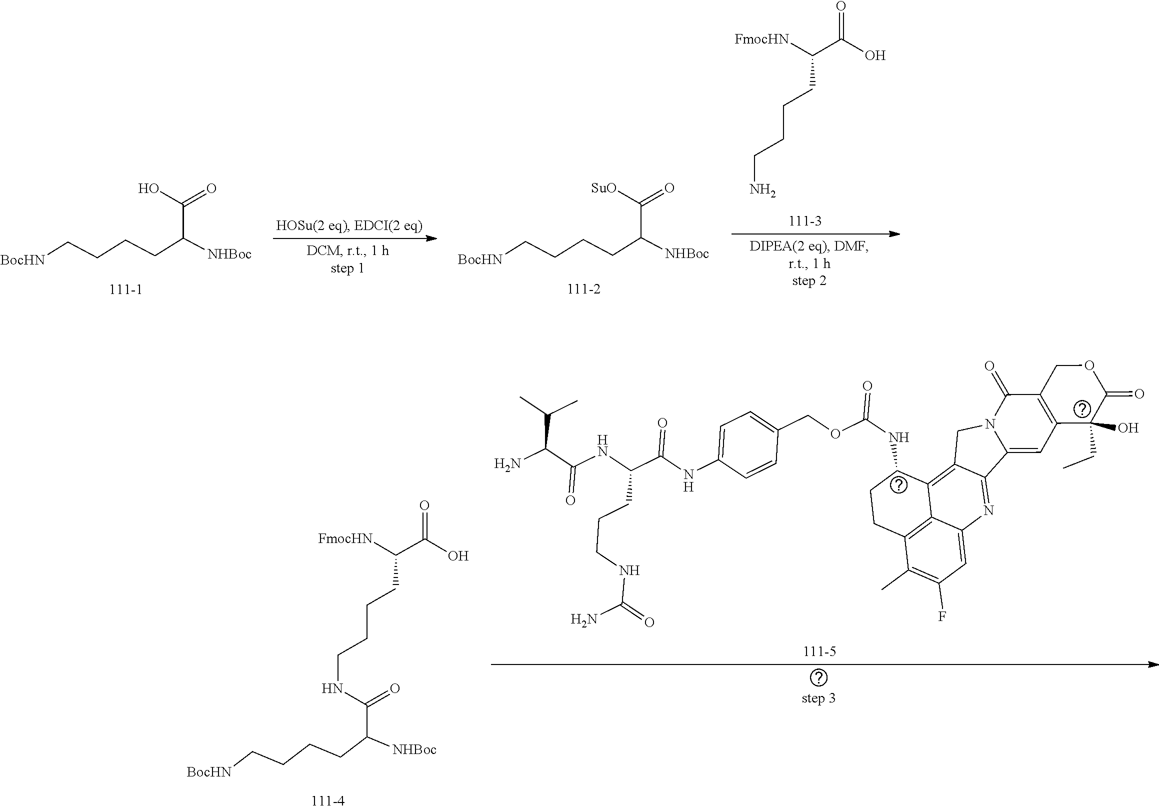

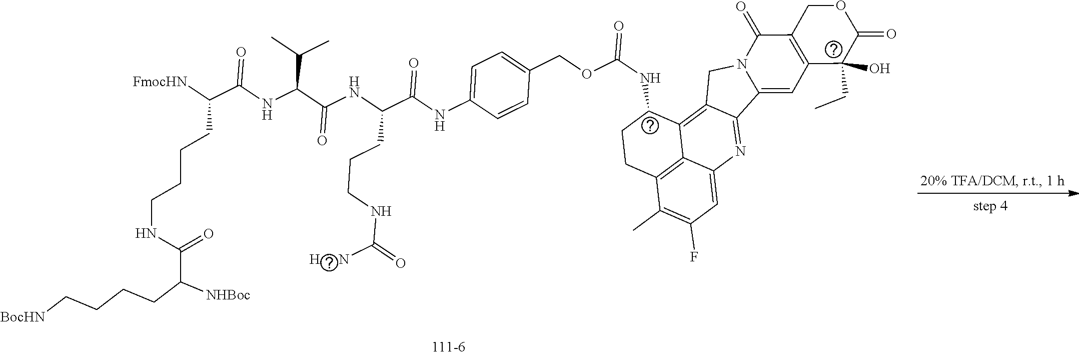

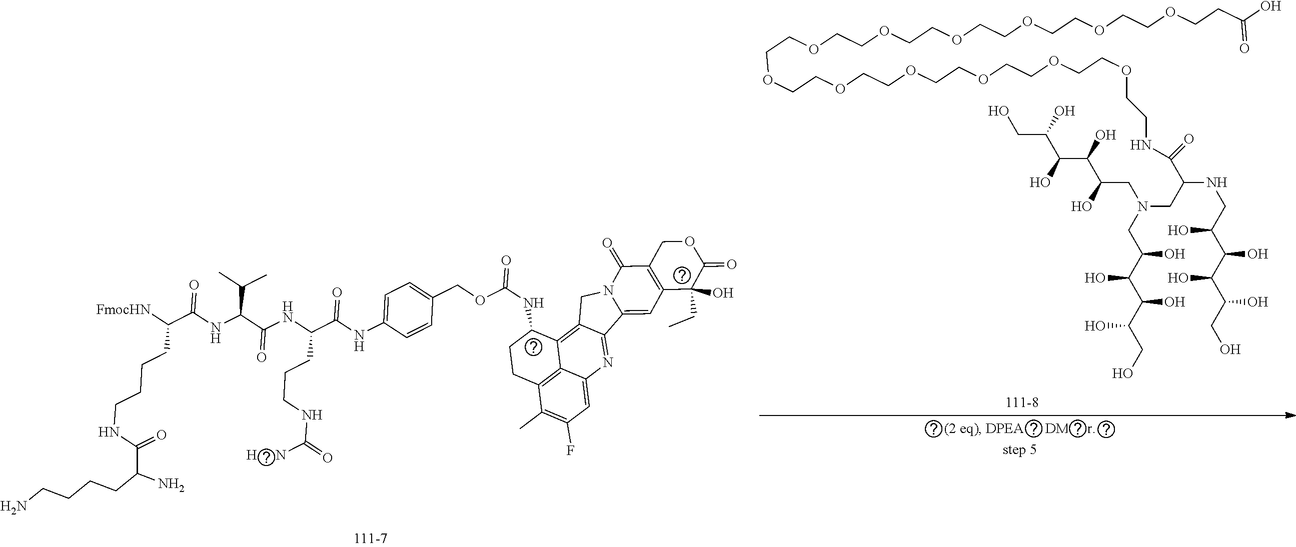

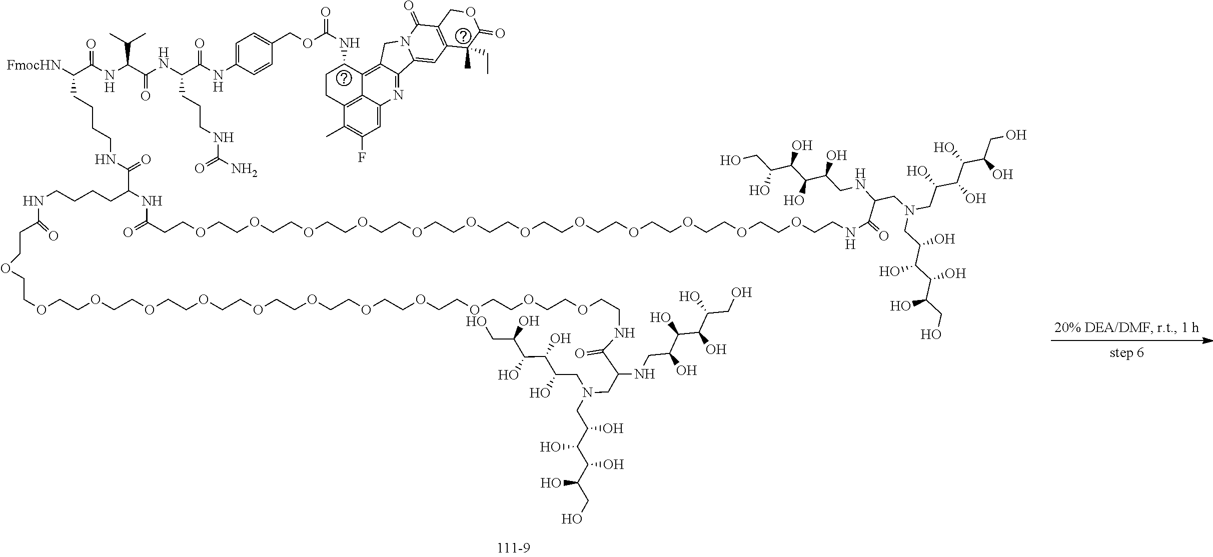

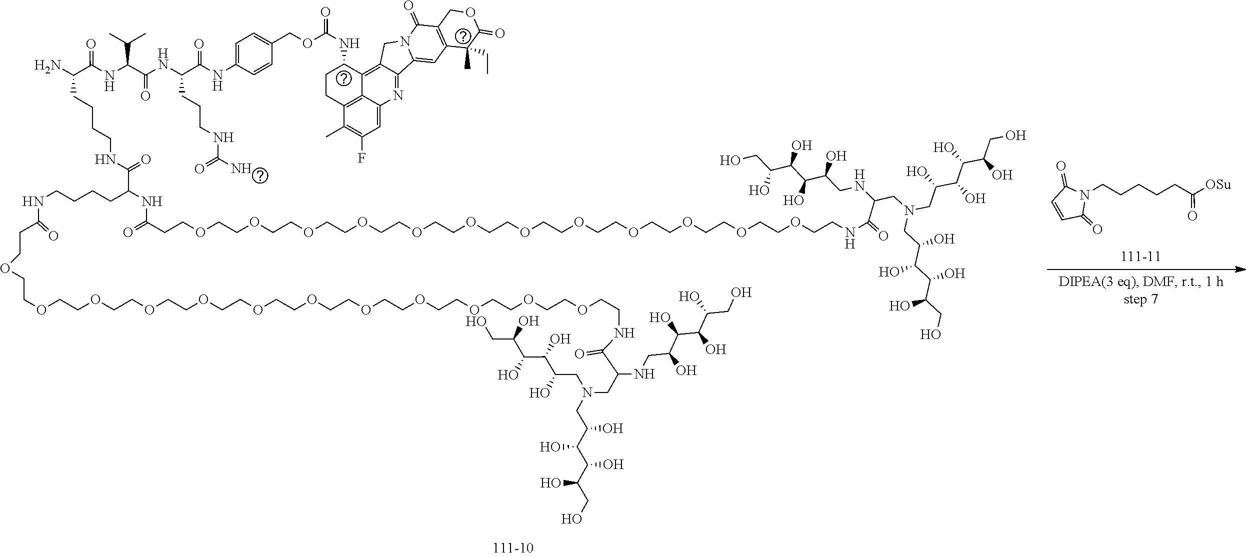

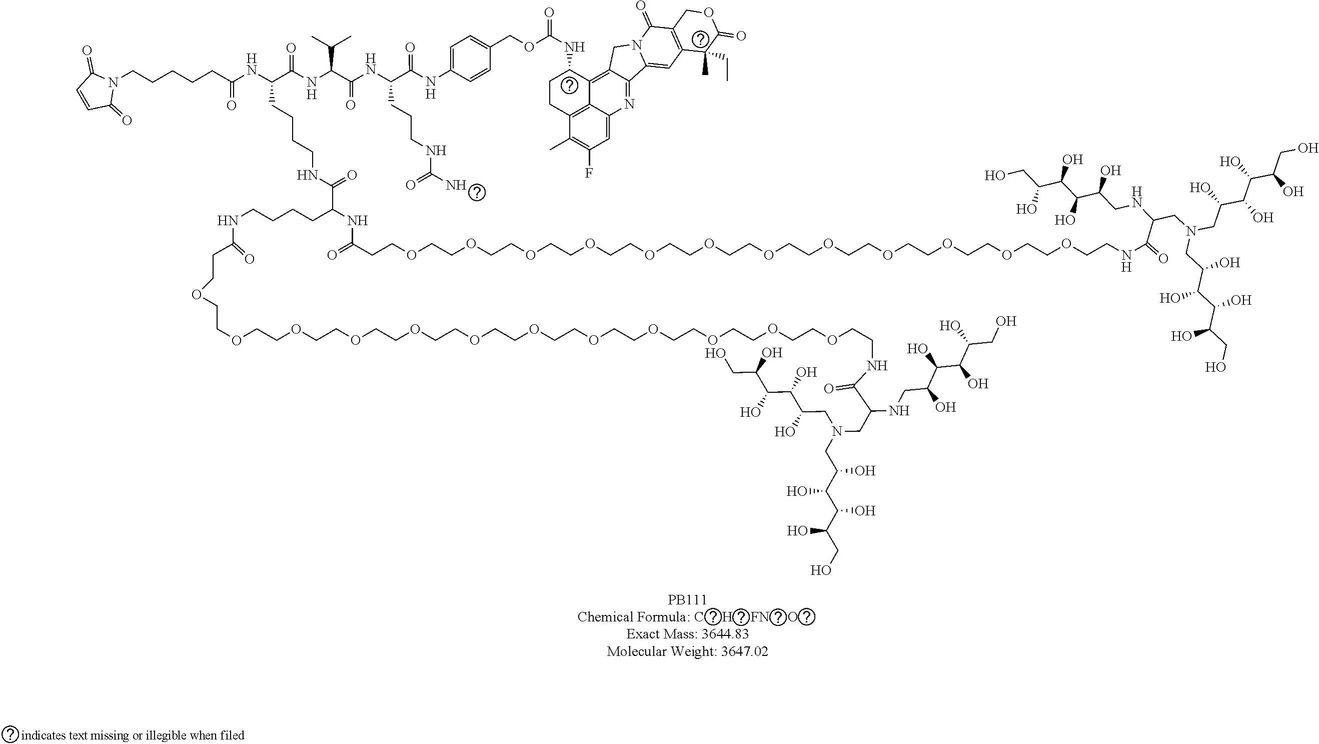

A Drug-Linker containing a PEG unit and a cleavable linker attached to exatecan (PB111 or LD111) was prepared as follows:

### Step 1

A solution of compound 111-1 (5 g, 14.433 mmol) and HOSu (3.3 g, 28.673 mmol) in anhydrous DCM (60 mL) was stirred at room temperature, then EDCI (5.5 g, 28.690 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (50 mL*2). The combined organic layer was dried over sodium sulfate and filtered, concentrated to dryness to yield compound 111-2 (4.8 g, 10.823 mmol, 75.00%) as a white solid, which was used as such in the next step. purity=85%-90%.

### Step 2

A solution of compound 111-2 (4.8 g, 10.823 mmol) and compound 111-3 (3.99 g, 10.829 mmol) in anhydrous DMF (20 mL) was stirred at room temperature, then DIPEA (2.8 g, 21.665 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 111-4 (5.1 g, 7.319 mmol, 67.64%) as a white solid, purity=90%-95%.

### Step 3

A solution of compound 111-4 (0.83 g, 1.191 mmol), compound 111-5 (1 g, 1.189 mmol) and HATU (0.45 g, 1.183 mmol) in anhydrous DMF (10 mL) was stirred at room temperature for 5 min, then DIPEA (0.46 g, 3.559 mmol) was added. The resulting solution was stirred for another 1 hr at r.t. until LCMS indicated complete reaction. The reaction solution was purified directly by reverse phase liquid chromatography to yield compound 111-6 (1.1 g, 0.724 mmol, 61.11%) as a white solid. purity=90%-95%.

### Step 4

A solution of compound 111-6 (1.1 g, 0.724 mmol) and TFA (2 mL) in anhydrous DCM (8 mL) was stirred at room temperature for 1 hr until LCMS indicated all starting amine was consumed and the desired product was detected. Then the solution was concentrated to dryness to yield compound 111-7 (0.9 g, 0.682 mmol, 94.24%) as a yellow oil, which was used as such in the next step. purity=85%-90%.

### Step 5

A solution of compound 111-7 (150 mg, 0.114 mmol), compound 111-8 (272 mg, 0.227 mmol) and HATU (87 mg, 0.229 mmol) in anhydrous DMF (5 mL) was stirred at room temperature for 5 min, then DIPEA (89 mg, 0.689 mmol) was added. The resulting solution was stirred for another 1 hr at r.t. until LCMS indicated complete reaction. The reaction solution was purified directly by reverse phase liquid chromatography to yield compound 111-9 (110 mg, 0.030 mmol, 26.38%) as a white solid. purity=90%-95%.

### Step 6

A solution of compound 111-9 (110 mg, 0.030 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 111-10 (75 mg, 0.022 mmol, 72.82%) as a colorless oil, which was used as such in the next step. purity=90%-95%.

### Step 7

A solution of compound 111-10 (75 mg, 0.022 mmol) and DIPEA (5.6 mg, 0.043 mmol) in anhydrous DMF (2 mL) was stirred at room temperature for 5 min, then a solution of compound 111-11 (10 mg, 0.032 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB111 (22 mg, 0.006 mmol, 27.78%) as a white solid. LCMS, m/z=912.79 (M/4+H)⁺. Drug linker PB111 can be used to make a conjugate such as PA111.

### Example 43: Preparation of the Drug-Linker (PB112) Containing a PEG Unit and a Cleavable Linker Attached to MMAE

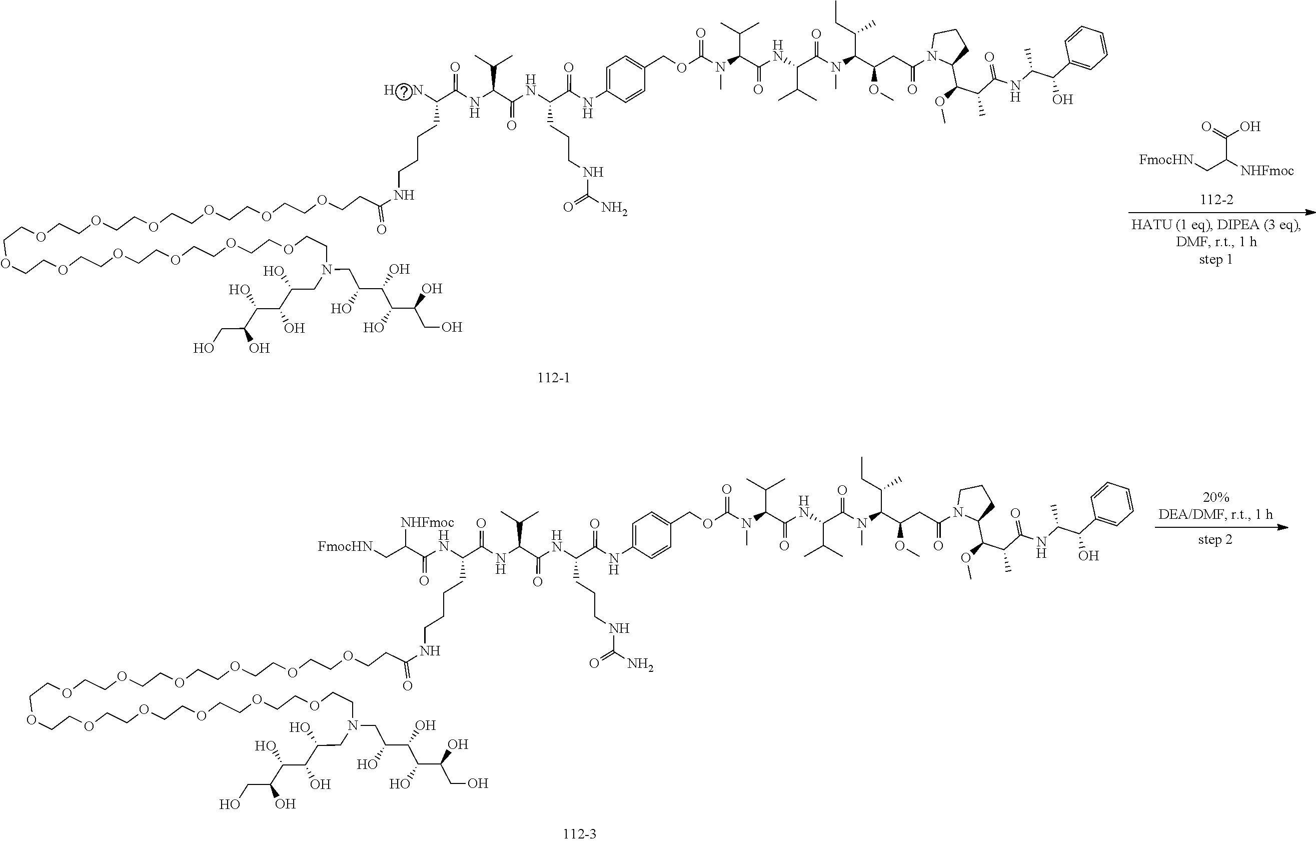

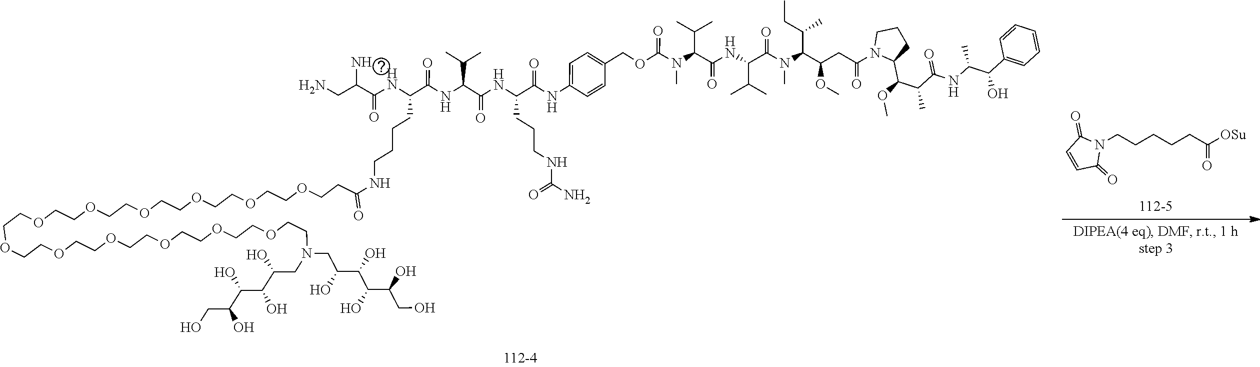

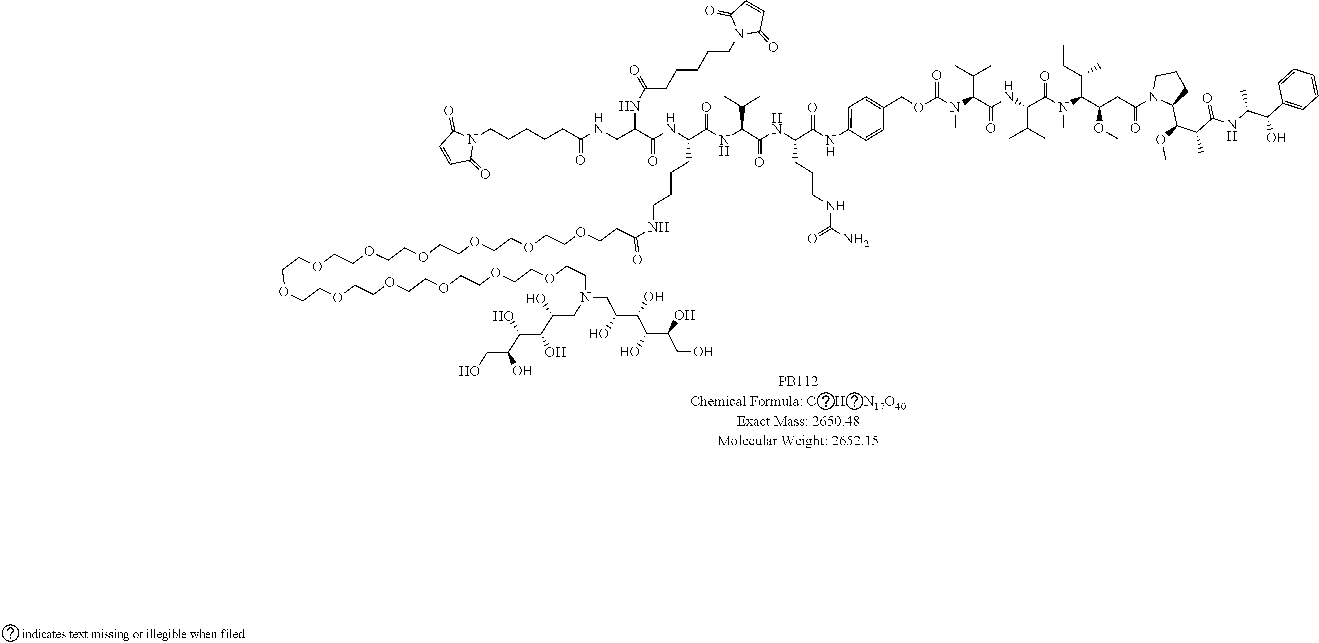 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)NC(=O)C(CNC(=O)CCCCCN2C(=O)C=CC2=O)NC(=O)CCCCCN2C(=O)C=CC2=O)C(C)C)cc1)C(C)C]

A Drug-Linker containing a PEG unit and a cleavable linker attached to MMAE (PB112) was prepared as follows:

### Step 1

A solution of compound 112-1 (100 mg, 0.046 mmol), compound 112-2 (25.2 mg, 0.046 mmol) and HATU (17.5 mg, 0.046 mmol) in anhydrous DMF (3 mL) was stirred at room temperature for 5 m, then DIPEA (17.8 mg, 0.138 mmol) was added. The resulting solution was stirred for another 1 hr at r.t. until LCMS indicated complete reaction. The reaction solution was purified directly by reverse phase liquid chromatography to yield compound 112-3 (85 mg, 0.031 mmol, 68.55%) as a white solid, purity=90%-95%.

### Step 2

A solution of compound 112-3 (85 mg, 0.031 mmol) and DEA (1 ml) in anhydrous DMF (4 mL) was stirred at room temperature for 15 until LCMS indicated all starting amine was consumed and the desired product was detected. Then the solution was concentrated to dryness to yield compound 112-4 (65 mg, 0.029 mmol, 91.47%) as a colorless oil, which was used as such in the next step. purity=90%-95%.

### Step 3

A solution of compound 112-4 (65 mg, 0.029 mmol) and DIPEA (14.8 mg, 0.116 mmol) in anhydrous DMF (2 mL) was stirred at room temperature for 5 min, then a solution of compound 112-5 (26.5 mg, 0.087 mmol) in anhydrous DMF (1 mL) was added dropwise by syringe over 5 min. The resulting solution was stirred for another 11 at r.t. until LCMS indicated all starting amine was consumed and the mass of desired product was detected. Then the reaction solution was purified by Prep-HPLC to yield PB112 (11 mg, 0.004 mmol, 14.47%) as a white solid. LCMS, m/z=885.71 (M/3+H)⁺. Drug linker PB112 can be used to make a conjugate such as PA112.

### Example 44: Preparation of the Drug-Linker (PB113) Containing a PEG Unit and a Cleavable Linker Attached to MMAE

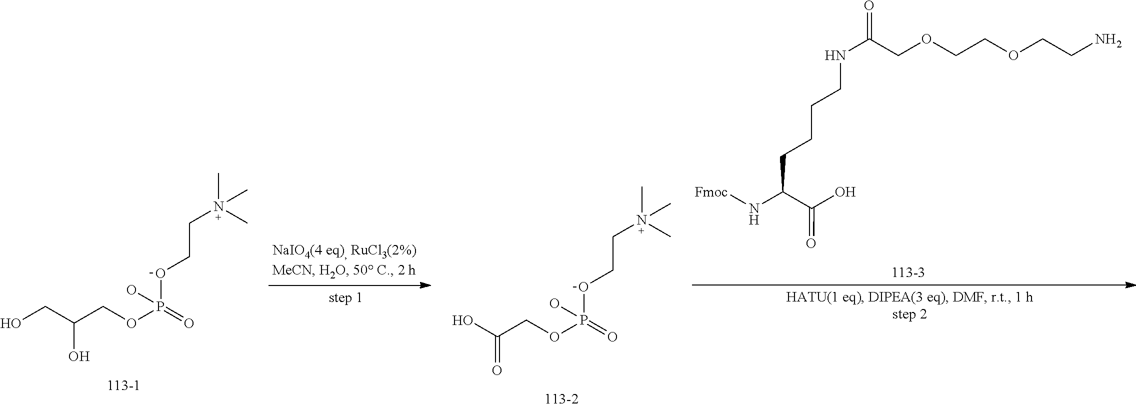

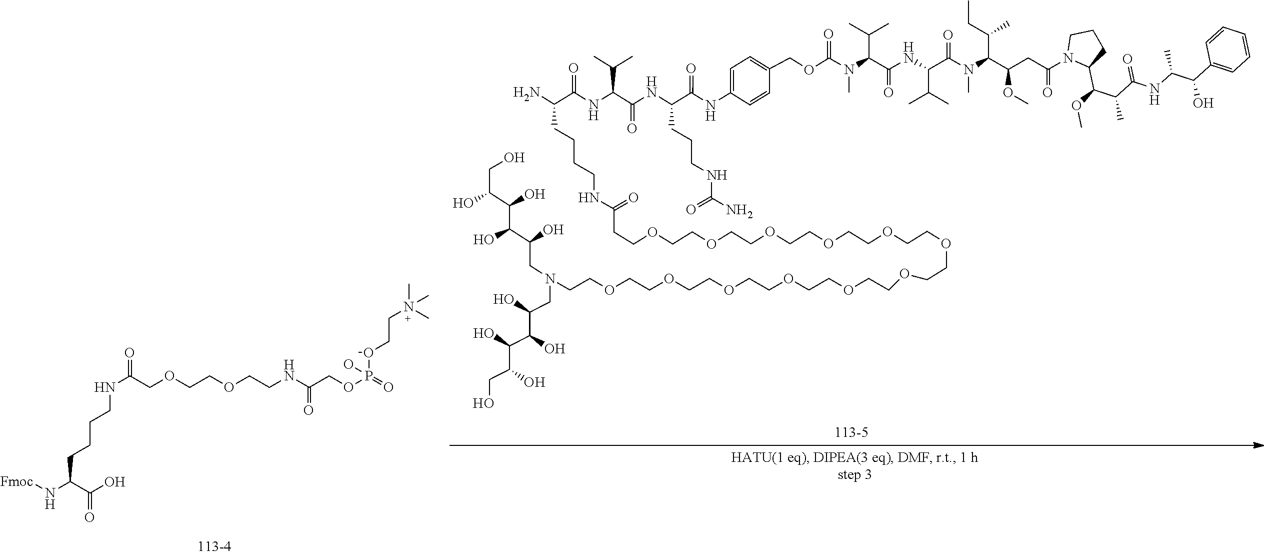

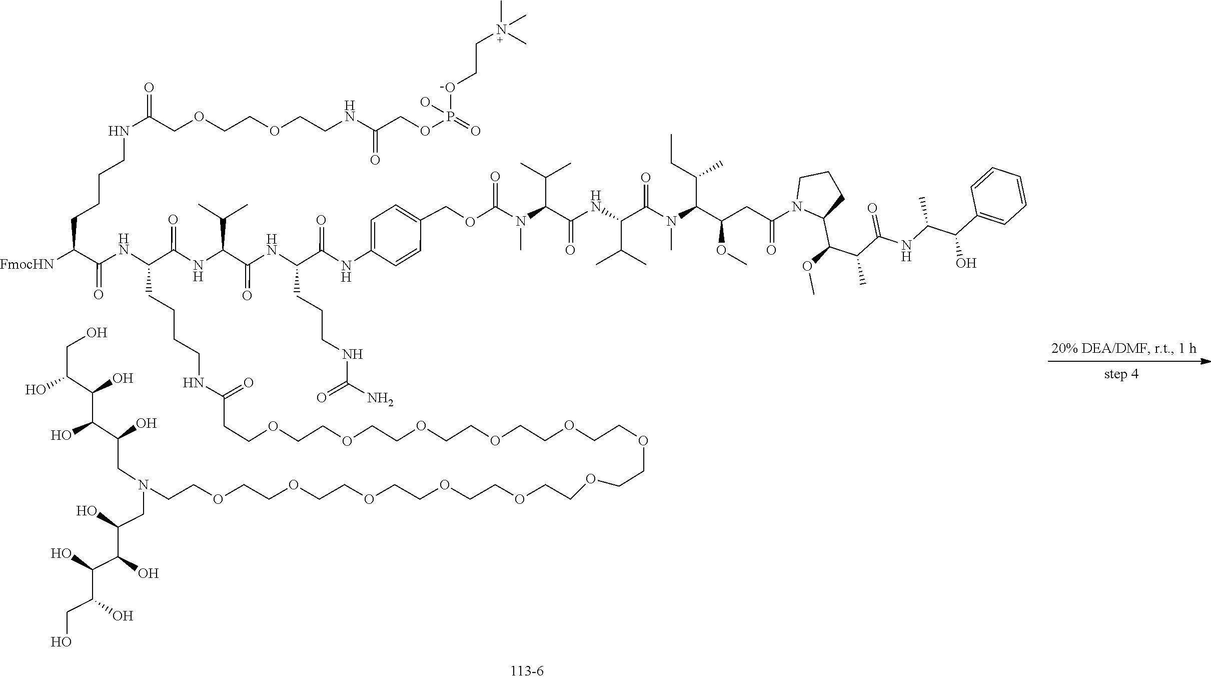

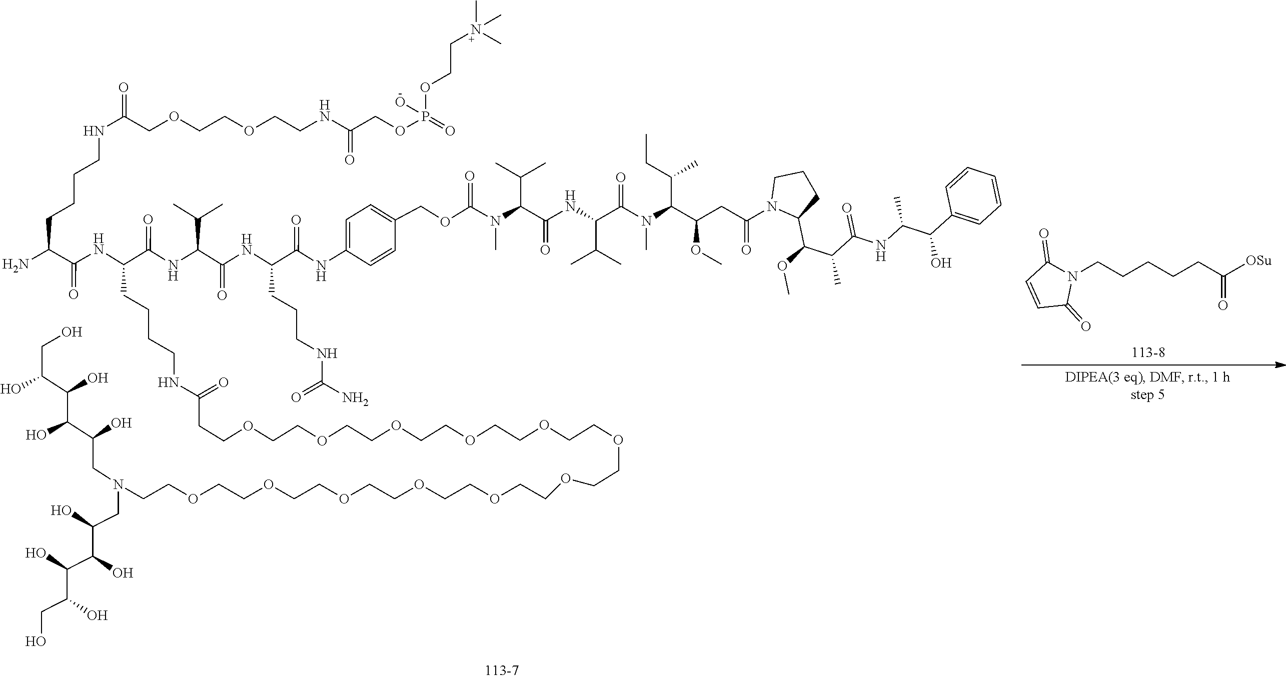

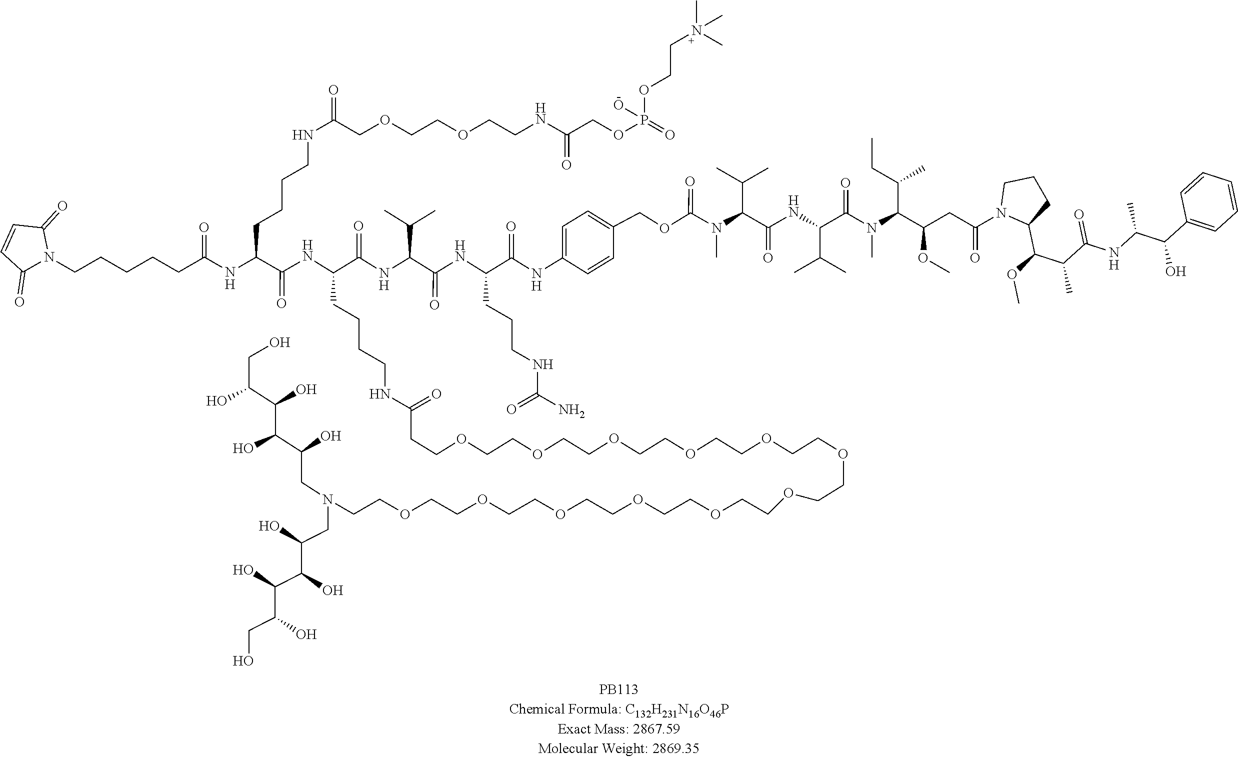

A Drug-Linker containing a PEG unit and a cleavable linker attached to MMAE (PB113) was prepared as follows:

### Step 1

A solution of compound 113-1 (20 g, 0.078 mol) and RuCl₃ (0.4 g, 2%) in MeCN (100 mL) and H₂O (200 mL) was stirred at room temperature for 5 min, then NaIO₄ (66.52 g, 0.311 mol) was added in several portions. The resulting solution was stirred for another 2 hr at 50° C. until LCMS indicated all starting amine was consumed and the desired product was detected. Methanol (20 mL) was added, the solution was evaporated and the residue was washed in acetonitrile (100 ml) at 5° C. for 30 minutes. The solution was filtered and the filtrate was concentrated to dryness to yield compound 113-2 (9.5 g, 0.039 mol, 50.67%) as a white solid. purity=50%-60%.

### Step 2

A solution of compound 113-2 (5 g, 0.021 mol), compound 113-3 (1 g, 1.95 mmol) and HATU (0.74 g, 1.95 mmol) in anhydrous DMF (20 mL) was stirred at room temperature for 5 min, then DIPEA (0.75 g, 5.85 mmol) was added. The resulting solution was stirred for another 1 hr at r.t. until LCMS indicated complete reaction. The reaction solution was purified directly by reverse phase liquid chromatography to yield compound 113-4 (410 mg, 0.556 mmol, 28.67%) as a white solid. purity=90%-95%.

### Step 3

A solution of compound 113-4 (20.6 mg, 0.028 mol), compound 113-5 (60 mg, 0.028 mmol) and HATU (10.5 mg, 0.028 mmol) in anhydrous DMF (2 mL) was stirred at room temperature for 5 min, then DIPEA (10.7 mg, 0.084 mmol) was added. The resulting solution was stirred for another 1 hr at r.t. until LCMS indicated complete reaction. The reaction solution was purified directly by reverse phase liquid chromatography to yield compound 113-6 (41 mg, 0.014 mmol, 51.38%) as a white solid. purity=90%-95%.

### Step 4

A solution of compound 113-6 (41 mg, 0.014 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 113-7 (31 mg, 0.012 mmol, 81.79%) as a yellow oil, which was used as such in the next step. purity=90%-95%.

### Step 5

A solution of compound 113-7 (31 mg, 0.012 mmol) and DIPEA (4.5 mg, 0.035 mmol) in anhydrous DMF (1 mL) was stirred at room temperature for 5 min, then a solution of compound 113-8 (5.4 mg, 0.017 mmol) in anhydrous DMF (1 mL) was added dropwise by syringe over 2 min. The resulting solution was stirred for another 1 hr at r.t. until LCMS indicated all starting amine was consumed and the mass of desired product was detected. The resulting solution was neutralized with formic acid to adjust the pH to 6-7. Then the reaction solution was purified by Prep-HPLC to yield PB113 (21 mg, 0.007 mmol, 63.25%) as a white solid. LCMS, m/z=957.42 (M/3+H)⁺. Drug linker PB113 can be used to make a conjugate such as PA113.

### Example 45: Preparation of the Drug-Linker (PB114) Attached to Exatecan

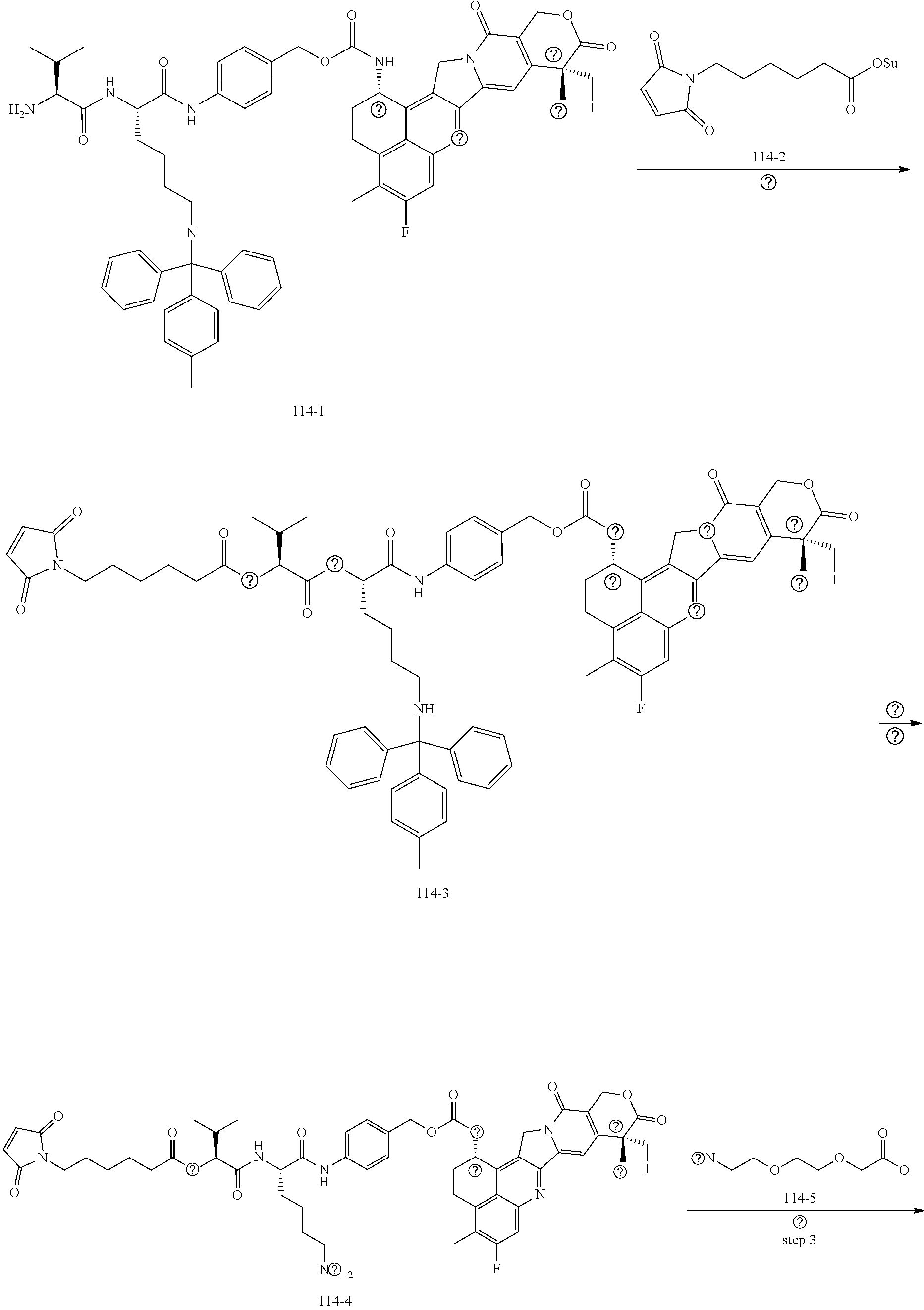

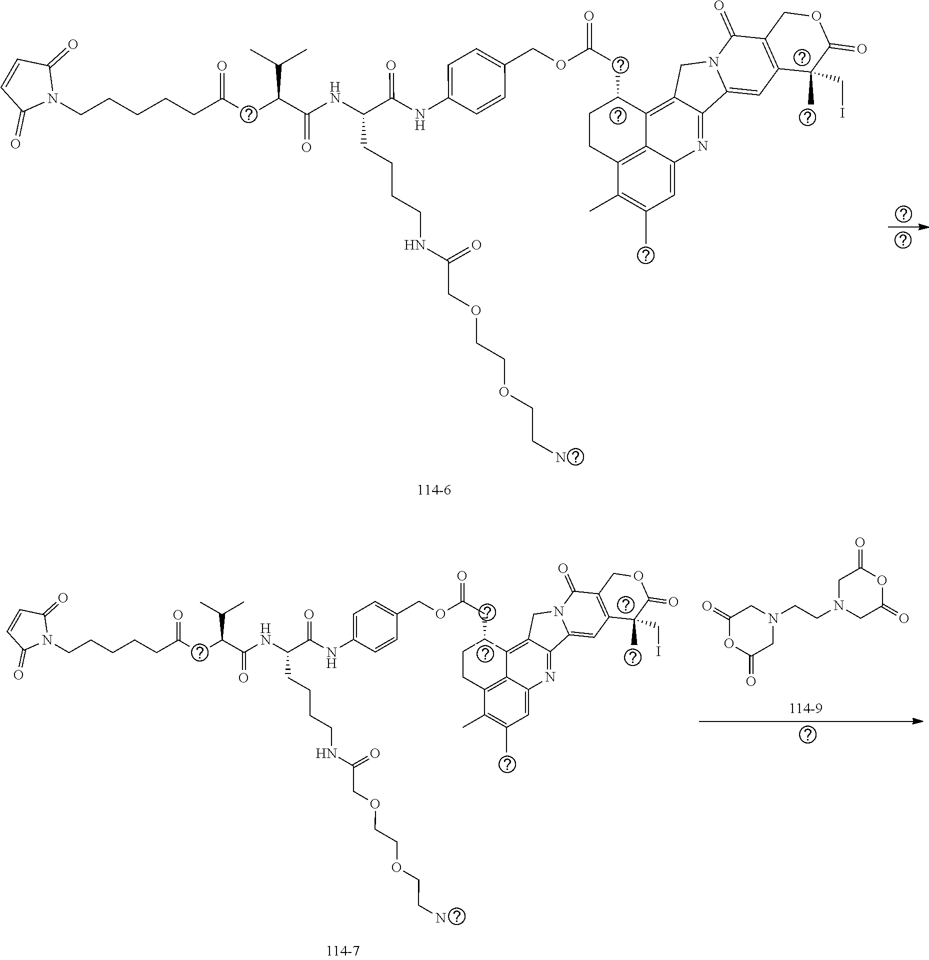

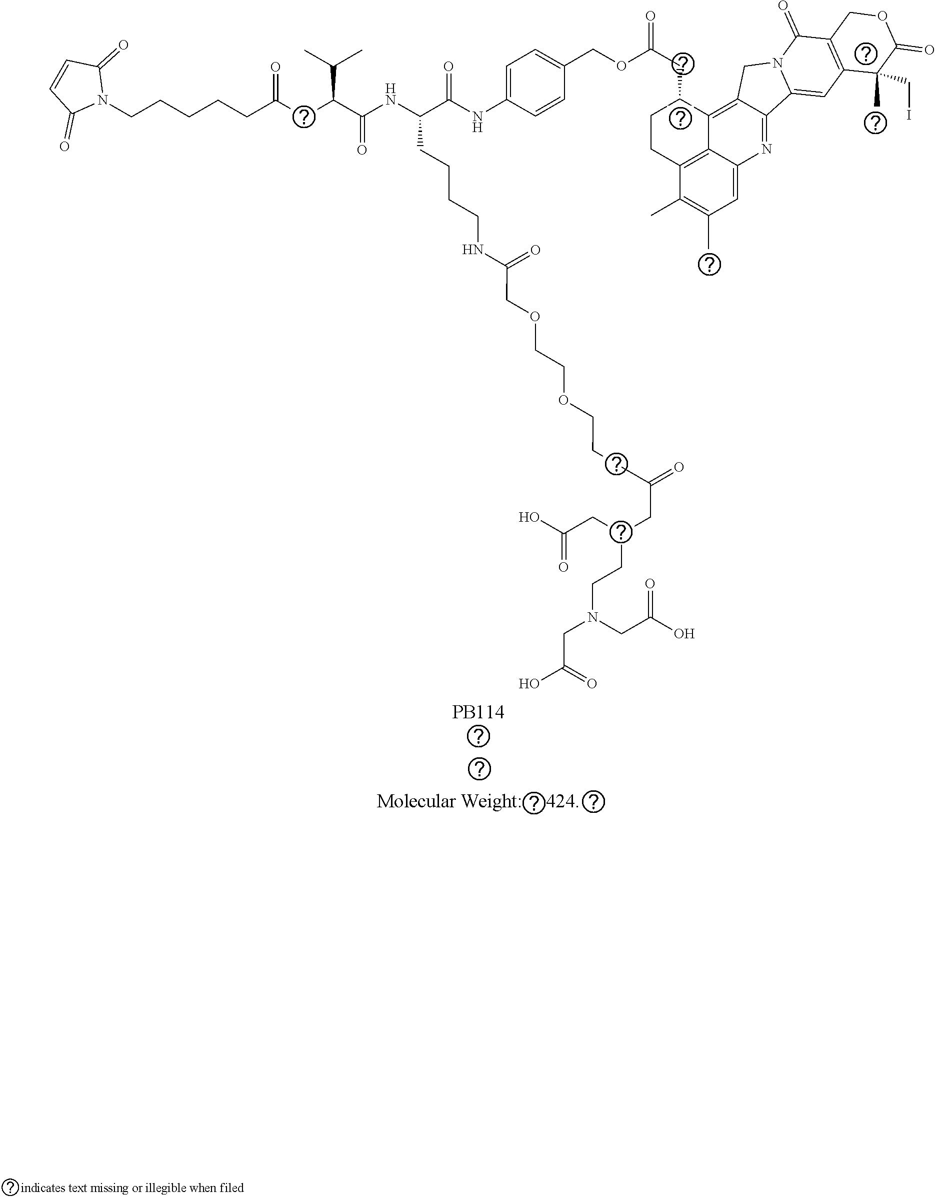

A Drug-Linker attached to exatecan (PB114) was prepared as follows:

### Step 1

A solution of compound 114-1 (5.8 g, 5.431 mmol) and DIPEA (2.1 g, 16.249 mmol) in anhydrous DMF (30 mL) was stirred at room temperature for 5 min, then a solution of compound 114-2 (2.5 g, 8.109 mmol) in anhydrous DMF (10 mL) was added dropwise by syringe over 5 min. The resulting solution was stirred for another 1 hr at r.t. until LCMS indicated all starting amine was consumed and the mass of desired product was detected. Then most of the DMF and DIPEA was evaporated and the residue was washed in acetonitrile (150 ml) at 5° C. for 30 minutes. The solution was filtered and the filter cake was washed with acetonitrile to yield compound 114-3 (5.2 g, 4.122 mmol, 75.91%) as a yellow solid. purity=85%-95%.

### Step 2

A solution of compound 114-3 (5.2 g, 4.122 mmol) and DCA (10 mL) in anhydrous DCM (90 mL) was stirred at room temperature for 2 hr until LCMS indicated all starting amine was consumed and the desired product was detected. The reaction solution was concentrated to dryness and purified directly by reverse phase liquid chromatography to yield compound 114-4 (2.5 g, 2.488 mmol, 60.24%) as a yellow solid. purity=85%-95%.

### Step 3

A solution of compound 114-4 (500 mg, 0.497 mmol), compound 114-5 (131 mg, 0.498 mmol) and HATU (189 mg, 0.497 mmol) in anhydrous DMF (5 mL) was stirred at room temperature for 5 min, then DIPEA (193 mg, 1.493 mmol) was added. The resulting solution was stirred for another 1 hr at r.t. until LCMS indicated complete reaction. The reaction solution was purified directly by reverse phase liquid chromatography to yield compound 114-6 (160 mg, 0.128 mmol, 25.72%) as a yellow solid. purity=85%-95%.

### Step 4

A solution of compound 114-6 (160 mg, 0.128 mmol) and TFA (1 mL) in anhydrous DCM (4 mL) was stirred at room temperature for 1 hr until LCMS indicated all starting amine was consumed and the desired product was detected. The reaction solution was purified directly by reverse phase liquid chromatography to yield compound 114-7 (50 mg, 0.043 mmol, 33.97%) as a yellow solid. purity=85%-95%.

### Step 5

A solution of compound 114-8 (16.7 mg, 0.065 mmol) and DIPEA (22.5 mg, 0.174 mmol) in anhydrous DMF (2 mL) was stirred at room temperature for 10 min, then a solution of compound 114-7 (50 mg, 0.043 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB114 (20 mg, 0.014 mmol, 32.31%) as a yellow solid. LCMS, m/z=713.19 (M/2+H)⁺. Drug linker PB114 can be used to make a conjugate such as PA114.

### Example 46: Preparation of the Drug-Linker (PB115) Attached to Exatecan

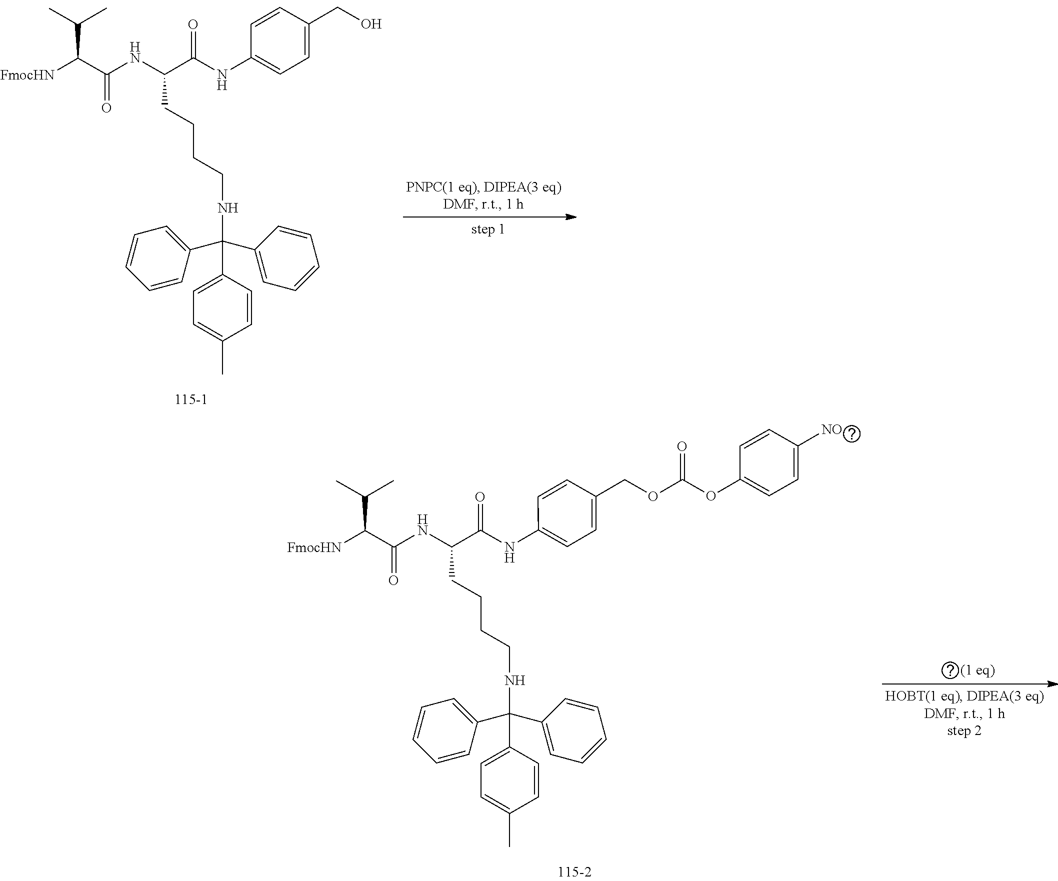

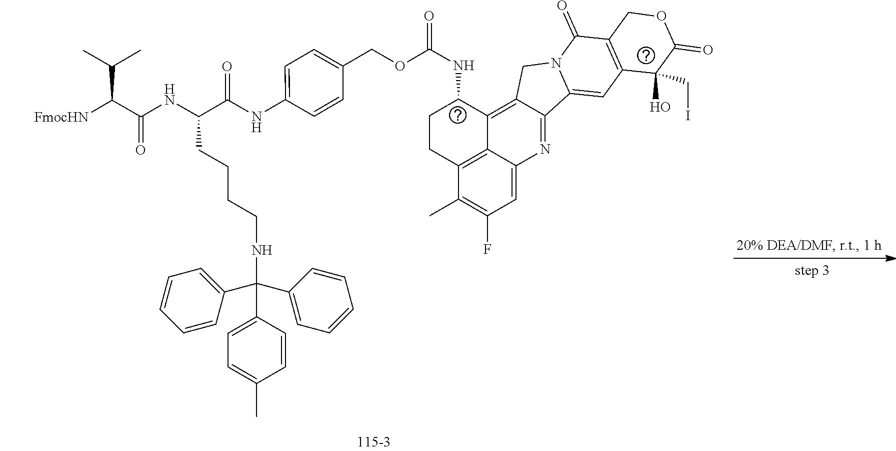

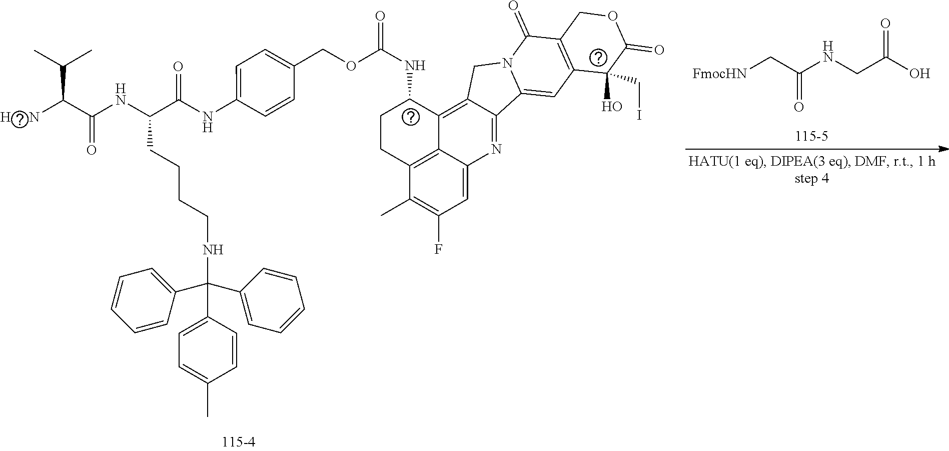

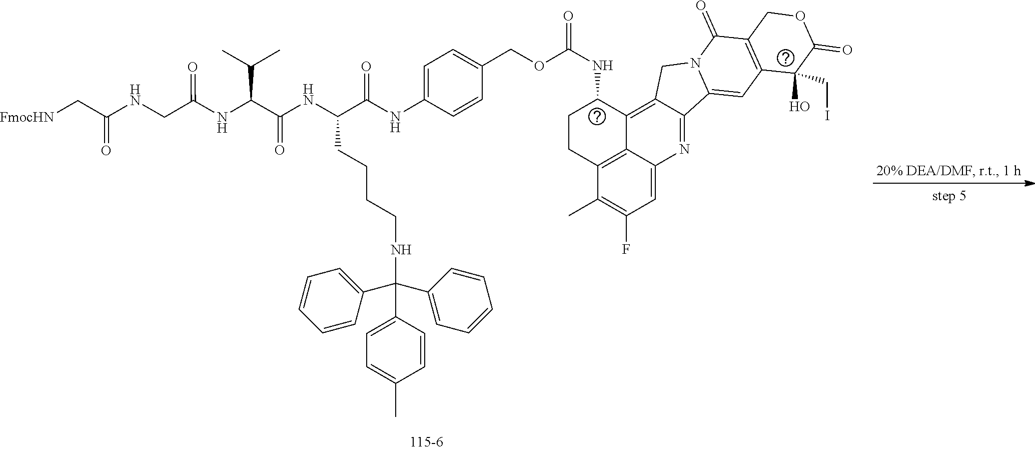

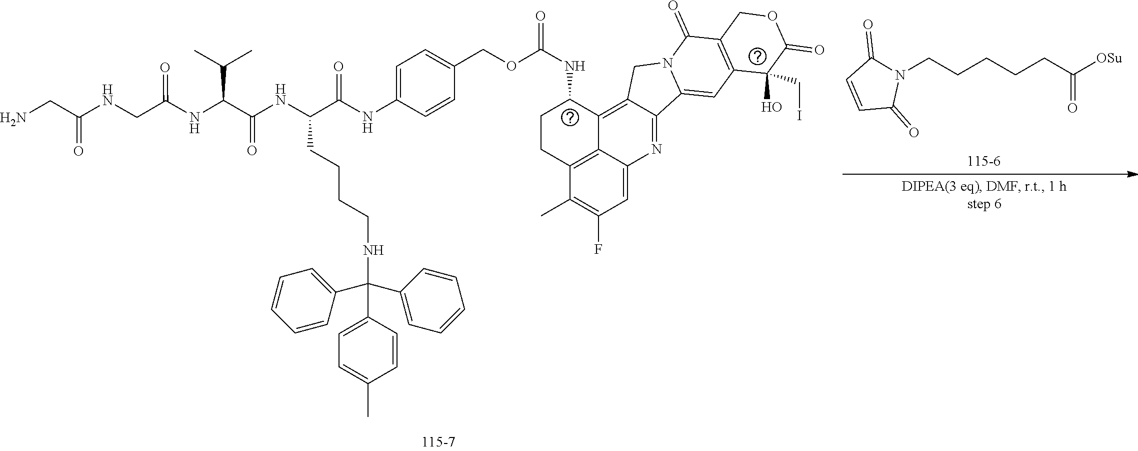

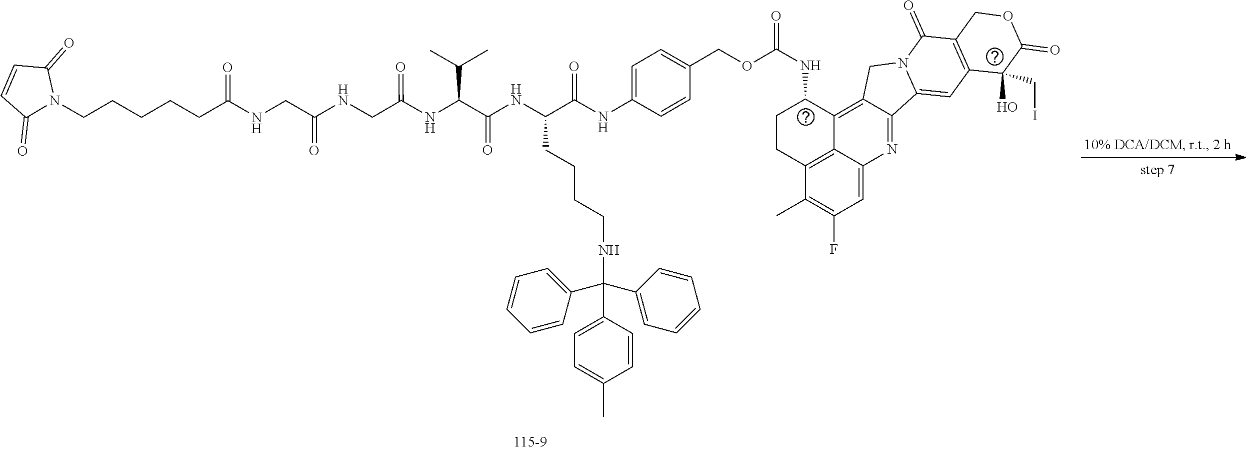

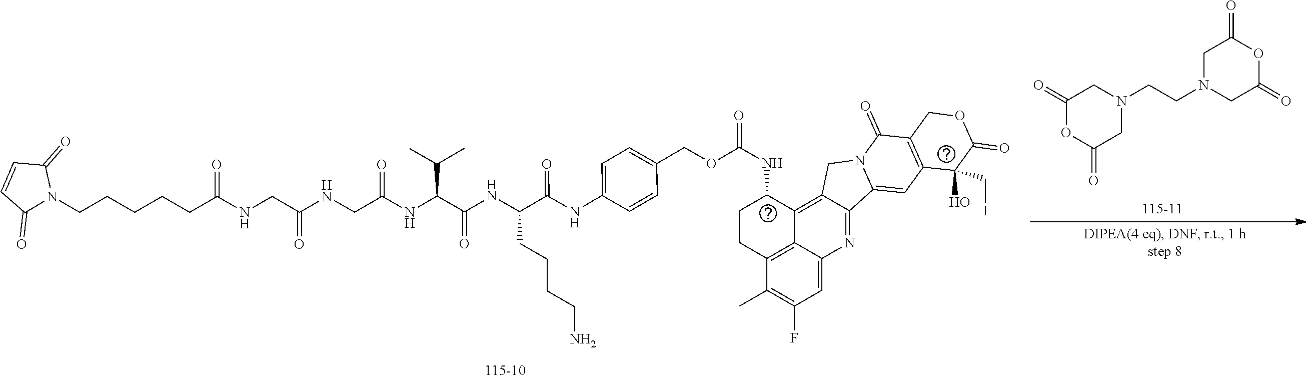

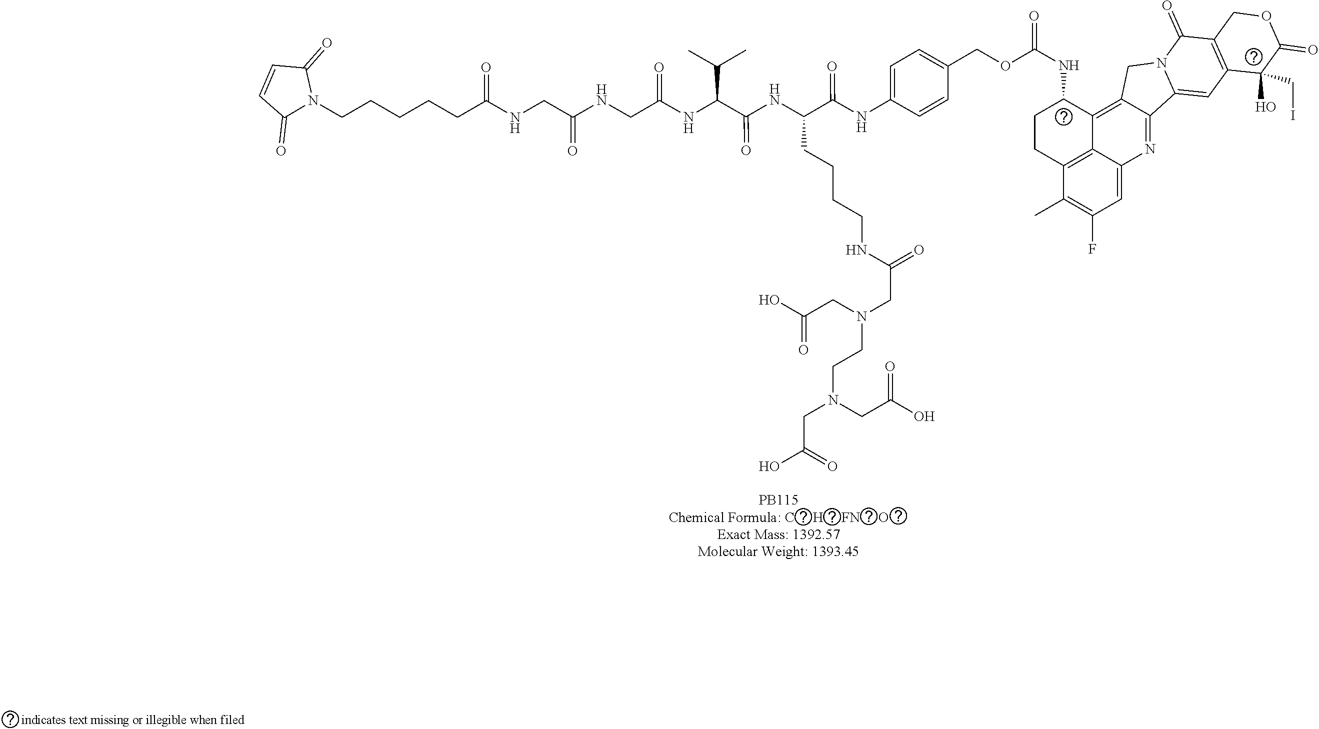

A Drug-Linker attached to exatecan (PB115) was prepared as follows:

### Step 1

A solution of compound 115-1 (10 g, 0.012 mol) and DIPEA (4.6 g, 0.036 mol) in anhydrous DMF (60 mL) was stirred at room temperature for 5 mi, then PNPC (3.67 g, 0.012 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (200 ml) at 5° C. for 30 minutes. The solution was filtered and the filter cake was washed with acetonitrile to yield compound 115-2 (8.4 g, 8.449 mmol, 70.06%) as a gray solid, purity=85%-95%.

### Step 2

A solution of compound 115-2 (8.4 g, 8.449 mmol), exatecan (4.5 g, 8.449 mmol) and HOBT (1.1 g, 8.449 mmol) in anhydrous DMF (50 mL) was stirred at room temperature, then DIPEA (3.3 g, 25.347 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (100 ml) at 5° C. for 30 minutes. The solution was filtered and the filter cake was washed with acetonitrile to yield compound 115-3 (9.3 g, 7.207 mmol, 85.32%) as a gray solid, purity=85%-95%.

### Step 3

A solution of compound 115-3 (9.3 g, 7.207 mmol) and DEA (10 mL) in anhydrous DMF (40 mL) was stirred at room temperature for 1 hr until LCMS indicated all starting amine was consumed and the desired product was detected. Then most of the DMF and DEA was evaporated and the residue was washed in acetonitrile (200 ml) at 5° C. for 30 minutes. The solution was filtered and the filter cake was washed with acetonitrile to yield compound 115-4 (5.8 g, 5.429 mmol, 75.32%) as a gray solid. purity=85%-95%.

### Step 4

A solution of compound 115-4 (1.6 g, 1.498 mmol), compound 115-5 (0.53 g, 1.498 mmol) and HATU (0.57 g, 1.498 mmol) in anhydrous DMF (10 mL) was stirred at room temperature for 5 min, then DIPEA (0.58 g, 4.494 mmol) was added. The resulting solution was stirred for another 1 hr at r.t. until LCMS indicated complete reaction. Then most of the DMF and DIPEA was evaporated and the residue was washed in acetonitrile (50 ml) at 5° C. for 30 minutes. The solution was filtered and the filter cake was washed with acetonitrile to yield compound 115-6 (1.7 g, 1.210 mmol, 80.95%) as a gray solid. purity=85%-95%.

### Step 5

A solution of compound 115-6 (1.7 g, 1.210 mmol) and DEA (2 mL) in anhydrous DMF (8 mL) was stirred at room temperature for 1 hr until LCMS indicated all starting amine was consumed and the desired product was detected. Then most of the DMF and DEA was evaporated and the residue was washed in acetonitrile (50 ml) at 5° C. for 30 minutes. The solution was filtered and the filter cake was washed with acetonitrile to yield compound 115-7 (1.1 g, 0.930 mmol, 76.92%) as a gray solid. purity=85%-95%.

### Step 6

A solution of compound 115-7 (1.1 g, 0.930 mmol) and DIPEA (0.36 g, 2.791 mmol) in anhydrous DMF (5 mL) was stirred at room temperature for 5 min, then a solution of compound 115-8 (0.43 g, 1.396 mmol) in anhydrous DMF (5 mL) was added dropwise by syringe over 2 min. The resulting solution was stirred for another 1 hr at r.t. until LCMS indicated all starting amine was consumed and the mass of desired product was detected. Then most of the DMF and DIPEA was evaporated and the residue was washed in acetonitrile (30 ml) at 5° C. for 30 minutes. The solution was filtered and the filter cake was washed with acetonitrile to yield compound 115-9 (1.1 g, 0.800 mmol, 85.94%) as a gray solid. purity=80%-90%.

### Step 7

A solution of compound 115-9 (1.1 g, 0.800 mmol) and DCA (2 mL) in anhydrous DCM (18 mL) was stirred at room temperature for 2 hr until LCMS indicated all starting amine was consumed and the desired product was detected. The reaction solution was concentrated to dryness and purified directly by reverse phase liquid chromatography to give 115-10 (310 mg, 0.277 mmol, 34.64%) as a white solid. purity=85%-95%.

### Step 8

A solution of compound 115-10 (141.9 mg, 0.554 mmol) and DIPEA (143.2 mg, 1.108 mmol) in anhydrous DMF (2 mL) was stirred at room temperature for 20 min, then a solution of 115-11 (310 mg, 0.277 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB115 (50 mg, 0.036 mmol, 12.95%) as a white solid. LCMS, m/z=697.66 (M/2+H)⁺. Drug linker PB115 can be used to make a conjugate such as PA115.

### Example 47: Preparation of the Drug-Linker (PB116) Attached to Palbociclib

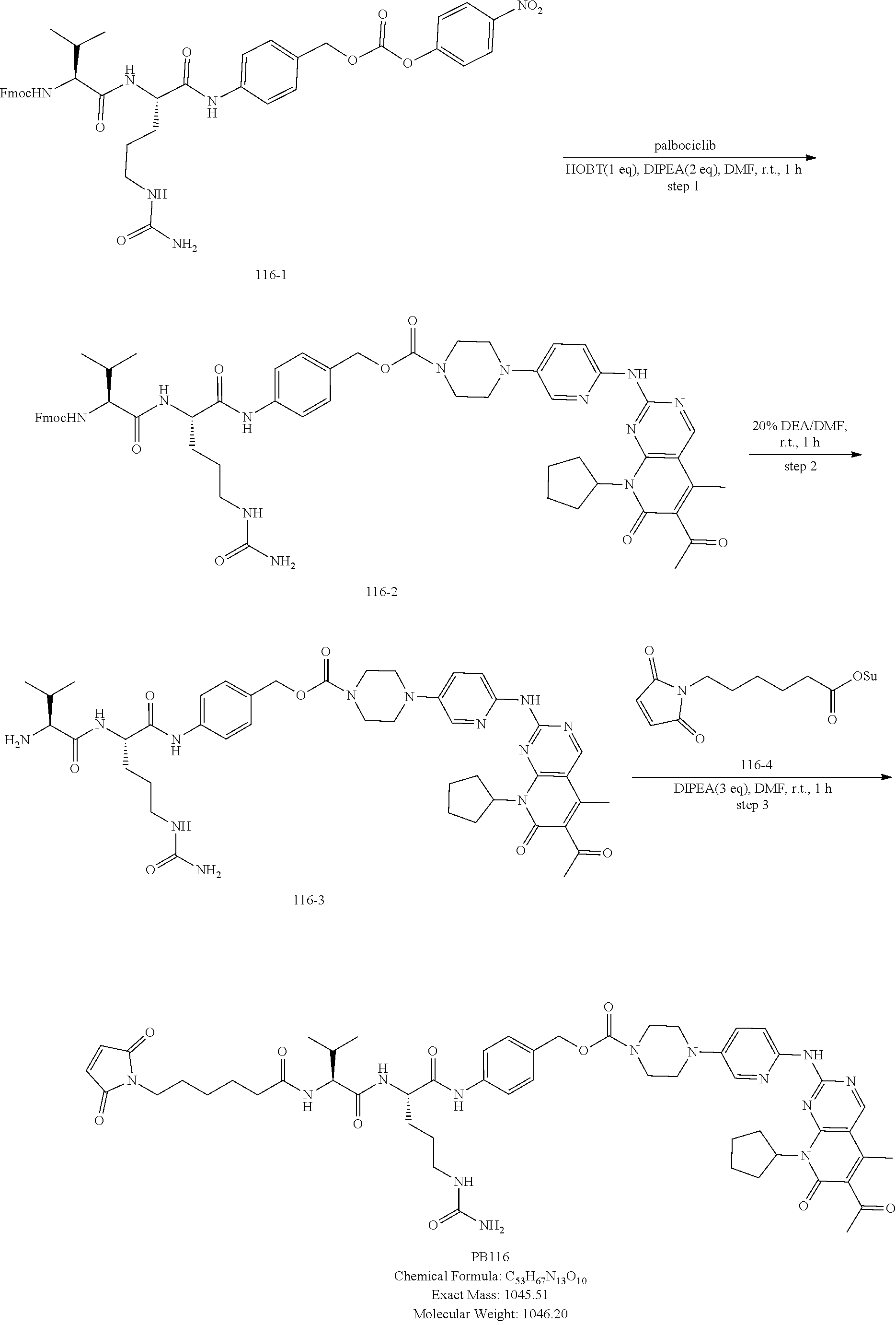

A Drug-Linker attached to palbociclib (PB116) was prepared as follows:

### Step 1

A solution of compound 116-1(685.4 mg, 0.894 mmol), palbociclib (400 mg, 0.894 mmol) and HOBT (120.8 mg, 0.894 mmol) in anhydrous DMF (60 m) was stirred at room temperature, then DIPEA (231 mg, 1.787 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (20 ml) at 5° C. for 30 minutes. The solution was filtered and the filter cake was washed with acetonitrile to yield compound 116-2 (720 mg, 0.670 mmol, 74.92%) as a yellow solid, purity=90%-95%.

### Step 2

A solution of compound 116-2 (720 mg, 0.670 mmol) and DEA (1 mL) in anhydrous DMF (4 mL) was stirred at room temperature for 1 hr until LCMS indicated all starting amine was consumed and the desired product was detected. Then most of the DMF and DEA was evaporated and the residue was washed in acetonitrile (50 ml) at 5° C. for 30 minutes. The solution was filtered and the filter cake was washed with acetonitrile to yield compound 116-3 (510 mg, 0.598 mmol, 89.32%) as a yellow solid. purity=95%-95%.

### Step 3

A solution of compound 116-3 (300 mg, 0.352 mmol) and DIPEA (136.5 mg, 1.056 mmol) in anhydrous DMF (3 mL) was stirred at room temperature for 5 min, then a solution of compound 116-4 (162.5 mg, 0.527 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield compound PB116 (210 mg, 0.201 mmol, 57.08%) as a yellow solid. LCMS, m/z=1046.59 (M+H)⁺. Drug linker PB116 can be used to make a conjugate such as PA116.

### Example 48: Preparation of the Drug-Linker (PB117) Containing a PEG Unit and a Cleavable Linker Attached to Palbociclib

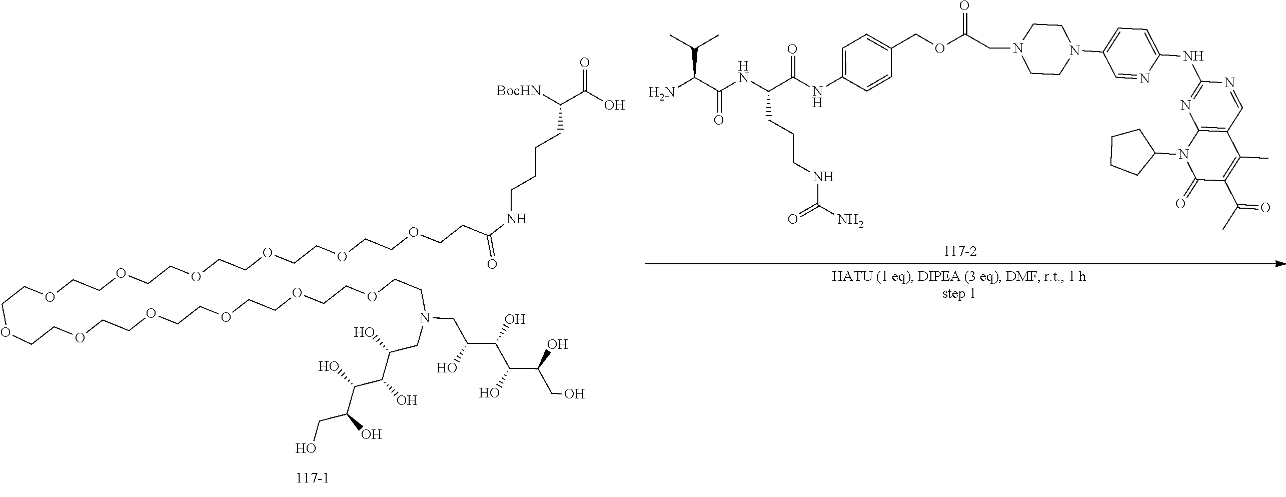

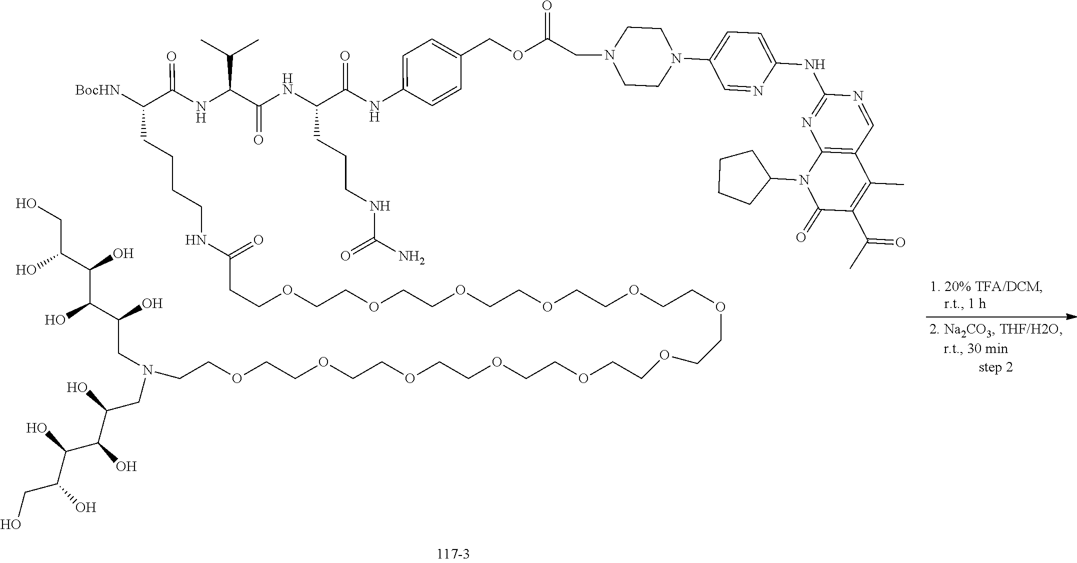

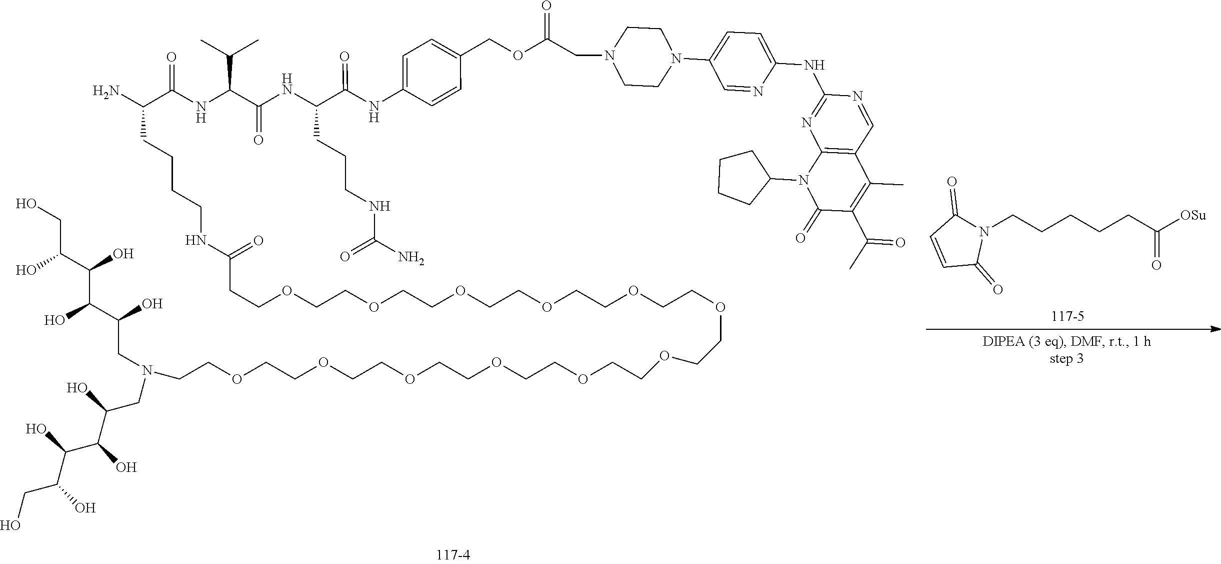

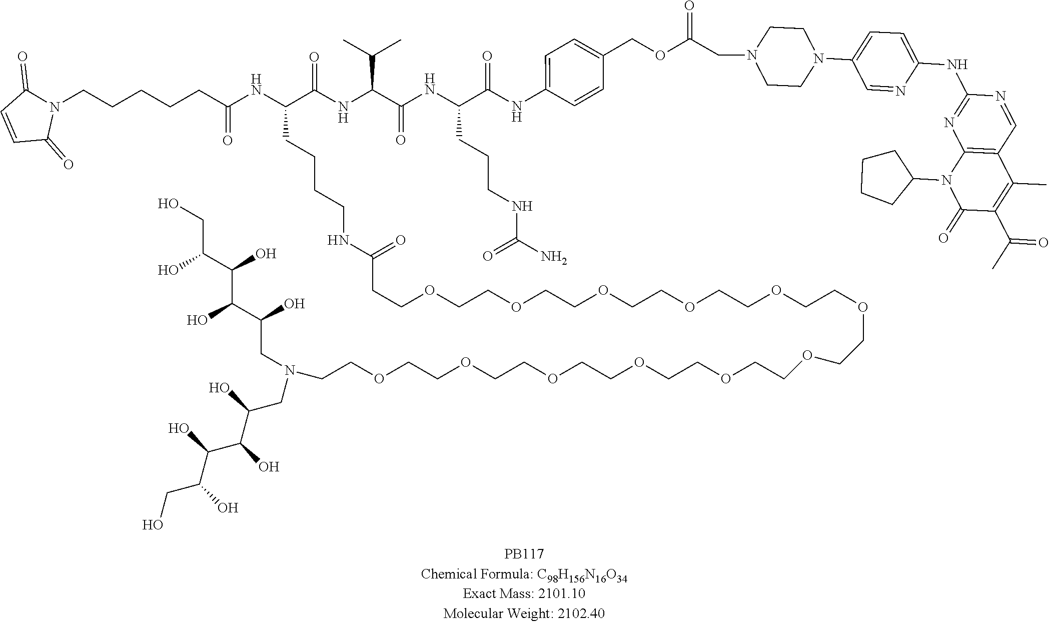

A Drug-Linker containing a PEG unit and a cleavable linker attached to palbociclib (PB117) was prepared as follows:

### Step 1

A solution of compound 117-1 (207 mg, 0.176 mmol), compound 117-2 (150 mg, 0.176 mmol) and HATU (66.8 mg, 0.176 mmol) in anhydrous DMF (2 mL) was stirred at room temperature for 5 min, then DIPEA (68.1 mg, 0.527 mmol) was added. The resulting solution was stirred for another 1 hr at r.t. until LCMS indicated complete reaction. The reaction solution was purified directly by reverse phase liquid chromatography to yield compound 117-3 (210 mg, 0.105 mmol, 59.49%) as a yellow solid. purity=90%-95%.

### Step 2

A solution of compound 117-3 (210 mg, 0.105 mmol) and TFA (2 mL) in anhydrous DCM (8 mL) was stirred at room temperature for 1 hr until LCMS indicated all starting amine was consumed and the desired product (m/z=637=1909/3+H) along with sugar-esterificated product (TFA was condensed with hydroxy group in sugar unit, mono-ester with m/z=(1909+96)/3+H=669) were formed. The completed reaction solution was condensed to dryness and then redissolved in THE (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 117-4 (120 mg, 0.063 mmol, 60.15%) as a yellow solid. purity=90%-95%.

### Step 3

A solution of compound 117-4 (120 mg, 0.063 mmol) and DIPEA (24.3 mg, 0.189 mmol) in anhydrous DMF (3 mL) was stirred at room temperature for 5 min, then a solution of compound 117-5 (29.0 mg, 0.094 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB117 (40 mg, 0.019 mmol, 30.30%) as a yellow solid. LCMS, m/z=1052.11 (M/2+H)⁺. Drug linker PB117 can be used to make a conjugate such as PA117.

### Example 49: Preparation of the Drug-Linker (PB118) Attached to T7-2

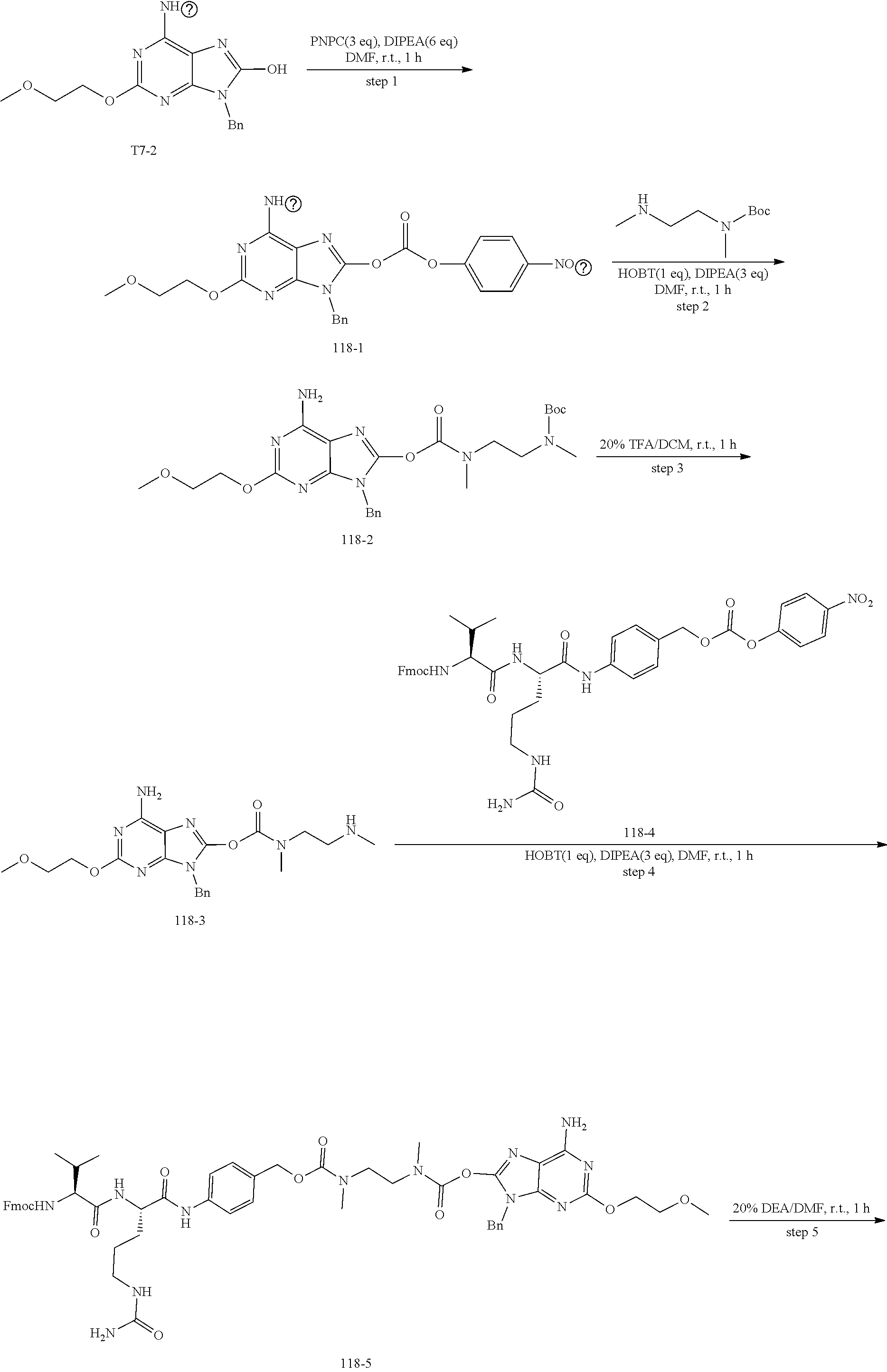

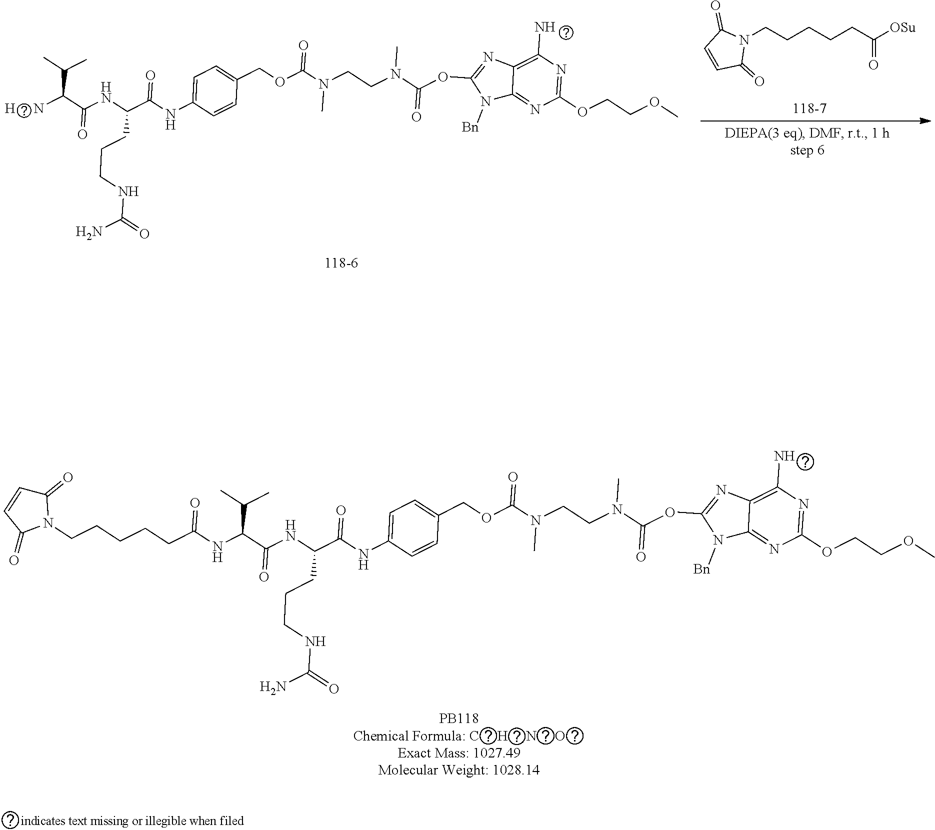

A Drug-Linker attached to T7-2 (PB118) was prepared as follows:

### Step 1

A solution of T7-2 (300 mg, 0.951 mmol) and DIPEA (737.8 mg, 5.706 mmol) in anhydrous DMF (6 mL) was stirred at room temperature for 5 min, then PNPC (868.26 mg, 2.853 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (50 ml) at 5° C. for 30 minutes. The solution was filtered and the filter cake was washed with acetonitrile to yield compound 118-1 (270 mg, 0.562 mmol, 59.08%) as a white solid. purity=90%-93%.

### Step 2

A solution of compound 118-1 (270 mg, 0.562 mmol), N-Boc-N,N′-dimethylethylenediamine (105.80 mg, 0.562 mmol) and HOBT (75.94 mg, 0.562 mmol) in anhydrous DMF (5 mL) was stirred at room temperature, then DIPEA (217.90 mg, 1.686 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 118-2 (210 mg, 0.397 mmol, 70.71%) as a white solid. purity=90%-95%.

### Step 3

A solution of compound 118-2 (210 mg, 0.397 mmol) and TFA (1 mL) in anhydrous DCM (4 mL) was stirred at room temperature for 1 hr until LCMS indicated all starting amine was consumed and the desired product was detected. Then the solution was concentrated to dryness to yield compound 118-3 (170 mg, 0.397 mmol, 100%) as a yellow oil, which was used as such in the next step. purity=85%-90%.

### Step 4

A solution of compound 118-3 (170 mg, 0.396 mmol), compound 118-4 (303.65 mg, 0.396 mmol) and HOBT (53.51 mg, 0.396 mmol) in anhydrous DMF (5 mL) was stirred at room temperature, then DIPEA (153.54 mg, 1.188 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 118-5 (95 mg, 0.090 mmol, 22.73%) as a white solid. purity=80%-90%.

### Step 5

A solution of compound 118-5 (95 mg, 0.090 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 118-6 (75 mg, 0.090 mmol, 100%) as a colorless oil, which was used as such in the next step. purity=80%-90%.

### Step 6

A solution of compound 118-6 (75 mg, 0.090 mmol) and DIPEA (34.83 mg, 0.270 mmol) in anhydrous DMF (1 mL) was stirred at room temperature for 5 min, then a solution of compound 118-7 (41.54 mg, 0.135 mmol) in anhydrous DMF (1 mL) was added dropwise by syringe over 2 min. The resulting solution was stirred for another 1 hr at r.t. until LCMS indicated all starting amine was consumed and the mass of desired product was detected. The resulting solution was neutralized with formic acid to adjust the pH to 6-7. Then the reaction solution was purified by Prep-HPLC to yield compound PB118 (10 mg, 0.001 mmol, 10.87%) as a white solid. LCMS, m/z=1028.55 (M+H)⁺. Drug linker PB118 can be used to make a conjugate such as PA118.

### Example 50: Preparation of the Drug-Linker (PB119) Containing a PEG Unit and a Cleavable Linker

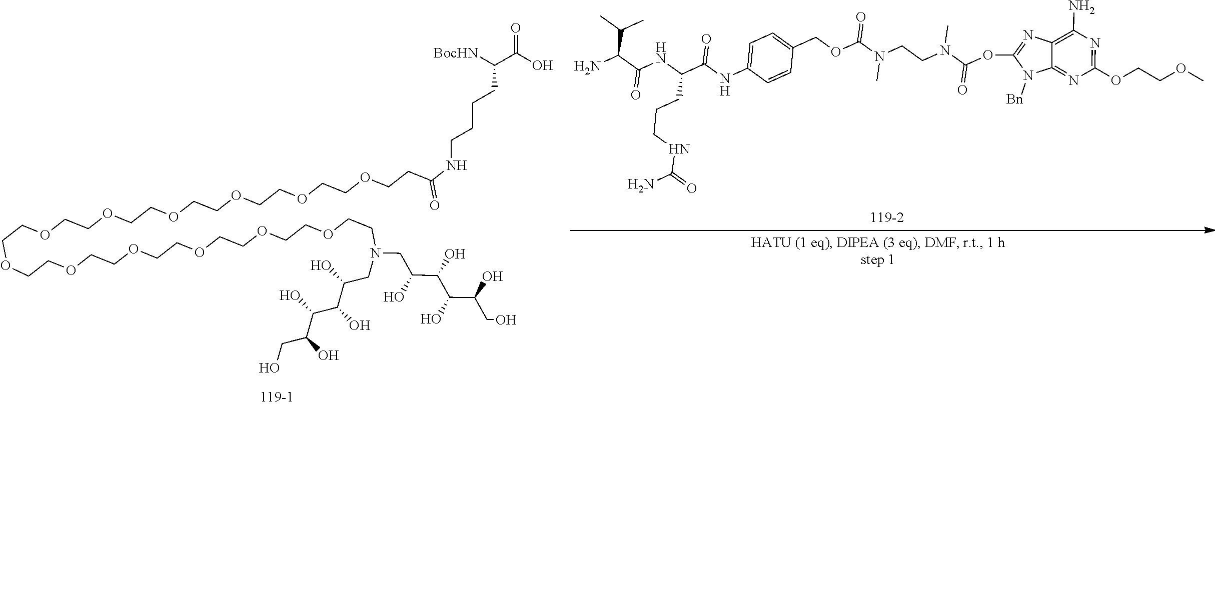

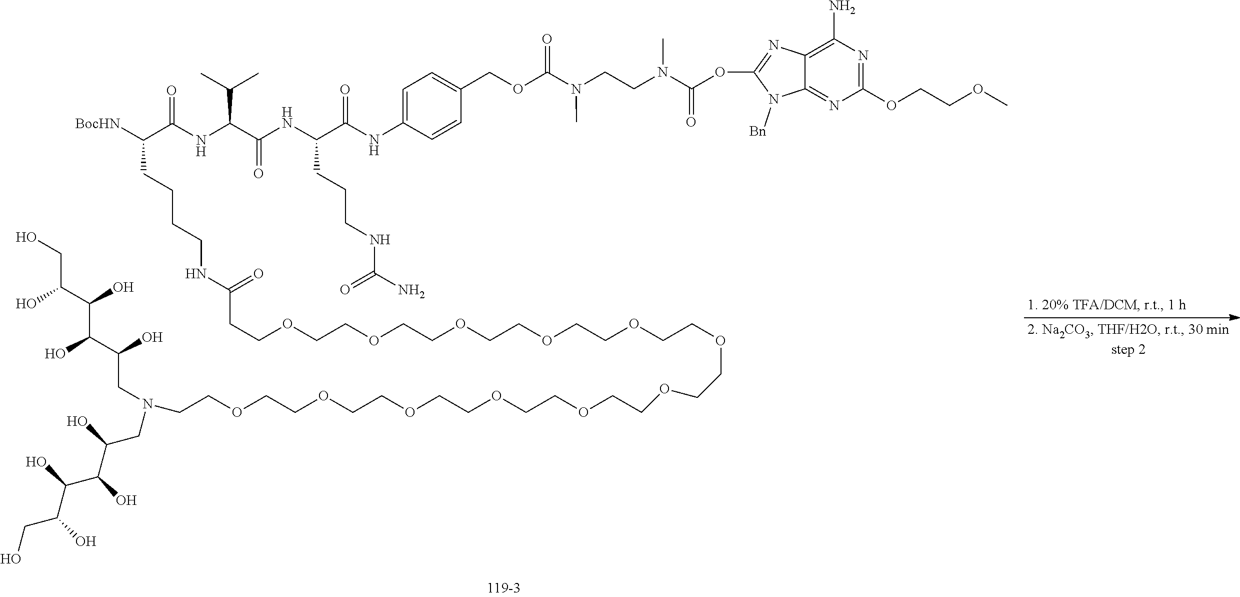

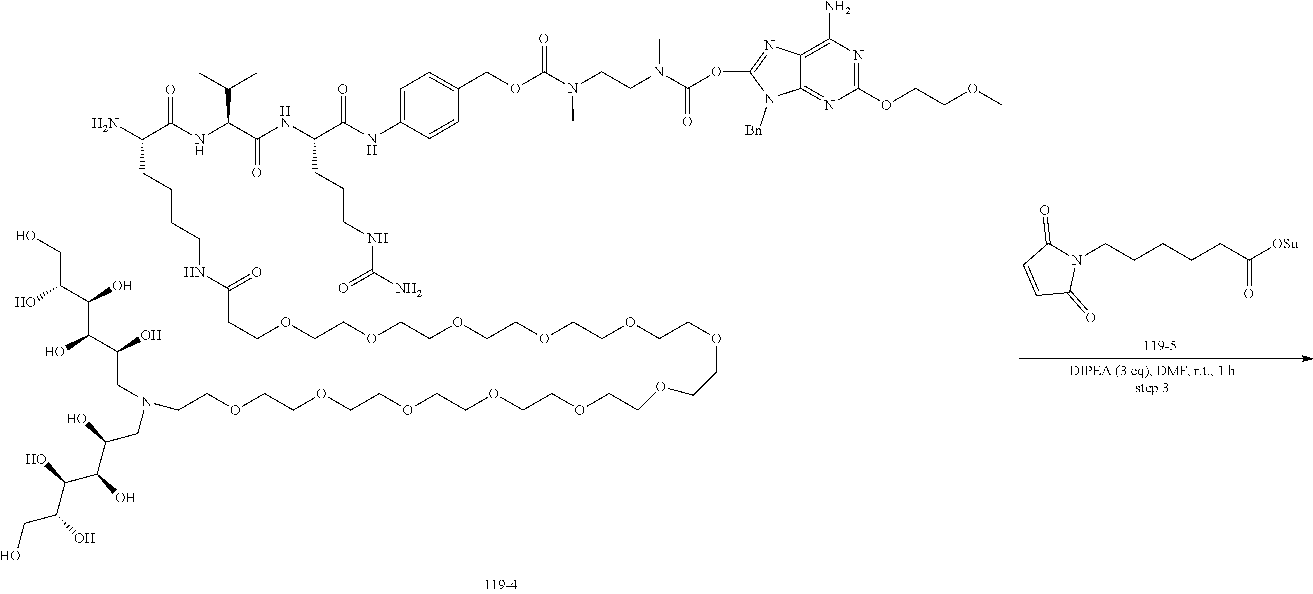

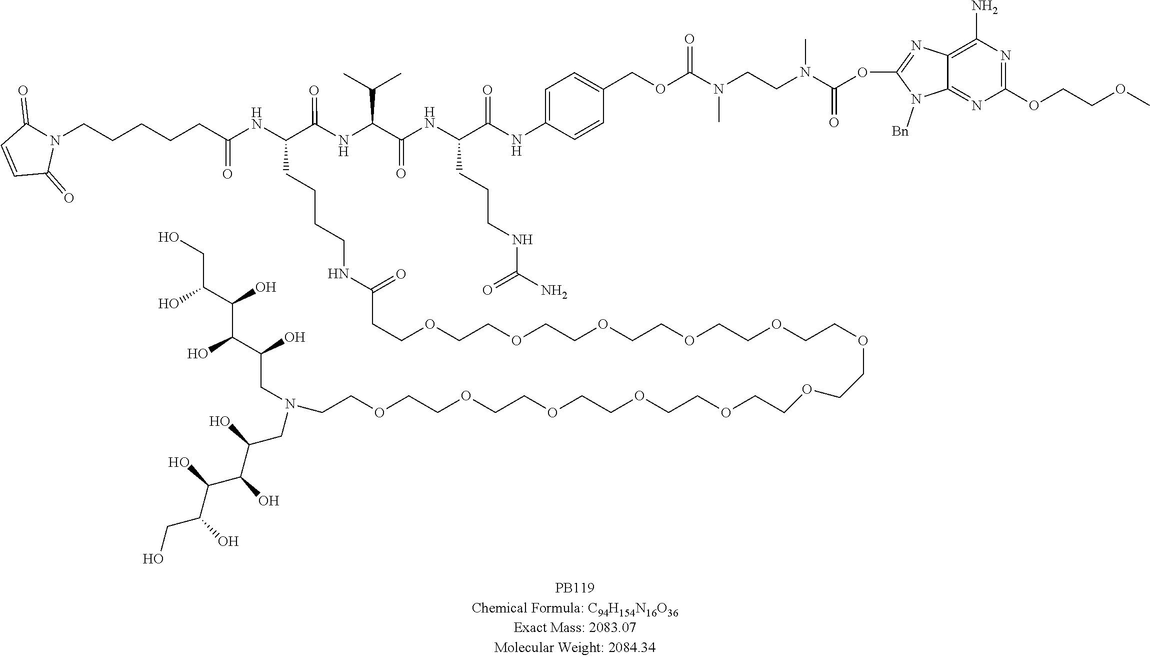

A Drug-Linker containing a PEG unit and a cleavable linker attached to T7-2 (PB119) was prepared as follows:

### Step 1

A solution of compound 119-1 (154.7 mg, 0.132 mmol), compound 119-2 (110 mg, 0.132 mmol) and HATU (50.1 mg, 0.132 mmol) in anhydrous DMF (2 mL) was stirred at room temperature for 5 min, then DIPEA (51.1 mg, 0.395 mmol) was added. The resulting solution was stirred for another 1 hr at r.t. until LCMS indicated complete reaction. The reaction solution was purified directly by reverse phase liquid chromatography to yield compound 119-3 (75 mg, 0.038 mmol, 28.63%) as a white solid, purity=90%-95%.

### Step 2

A solution of compound 119-3 (75 mg, 0.038 mmol) and TFA (1 mL) in anhydrous DCM (4 mL) was stirred at room temperature for 1 hr until LCMS indicated all starting amine was consumed and the desired product (m/z=631=1891/3+H) along with sugar-esterificated product (TFA was condensed with hydroxy group in sugar unit, mono-ester with m/z=(1891+96)/3+H=663) were formed. The completed reaction solution was condensed to dryness and then redissolved in THF (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 119-4 (41 mg, 0.022 mmol, 57.75%) as a white solid, purity=80%-85%.

### Step 3

A solution of compound 119-4 (41 mg, 0.022 mmol) and DIPEA (8.5 mg, 0.066 mmol) in anhydrous DMF (2 mL) was stirred at room temperature for 5 min, then a solution of compound 119-5 (10.0 mg, 0.033 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield compound PB119 (9 mg, 0.004 mmol, 19.92%) as a white solid. LCMS, m/z=1043.05 (M/2+H)⁺. Drug linker PB119 can be used to make a conjugate such as PA119.

### Example 51: Preparation of the Drug-Linker (PB120) Containing a PEG Unit and a Cleavable Linker Attached to T7-1

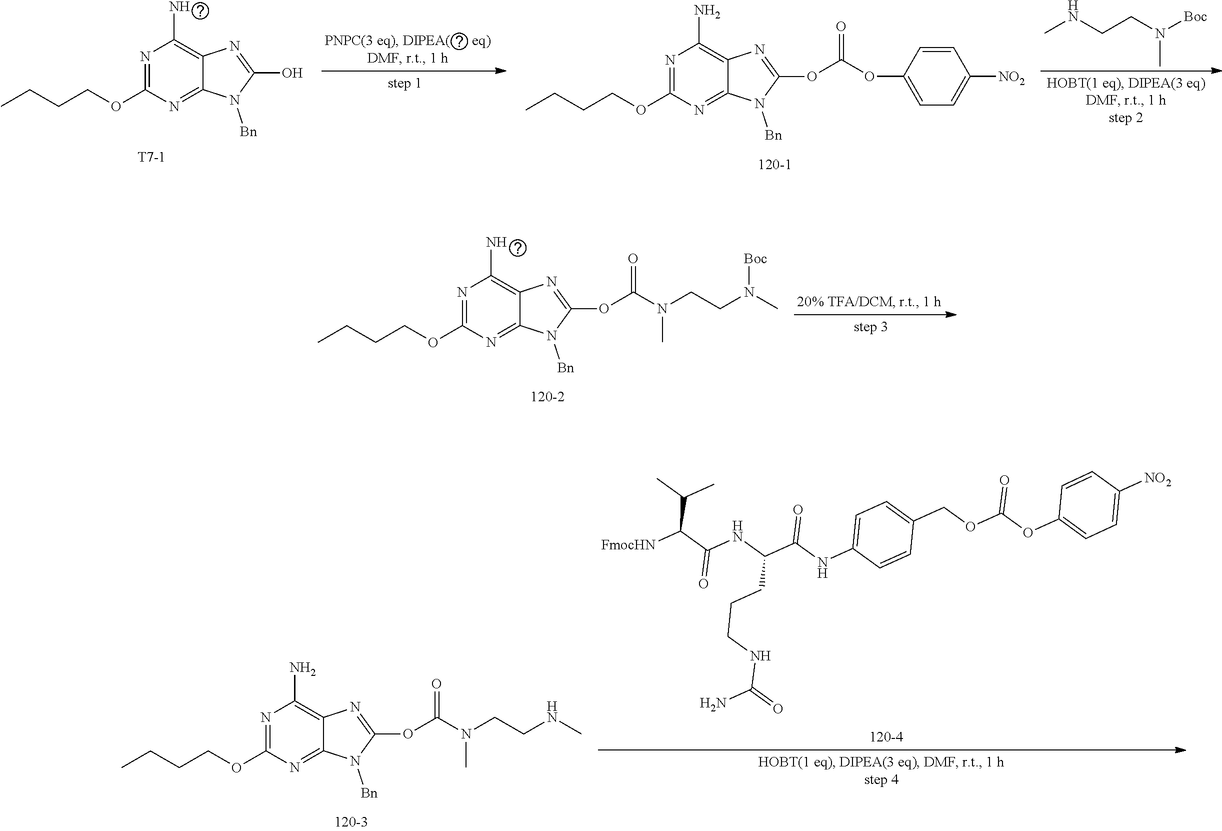

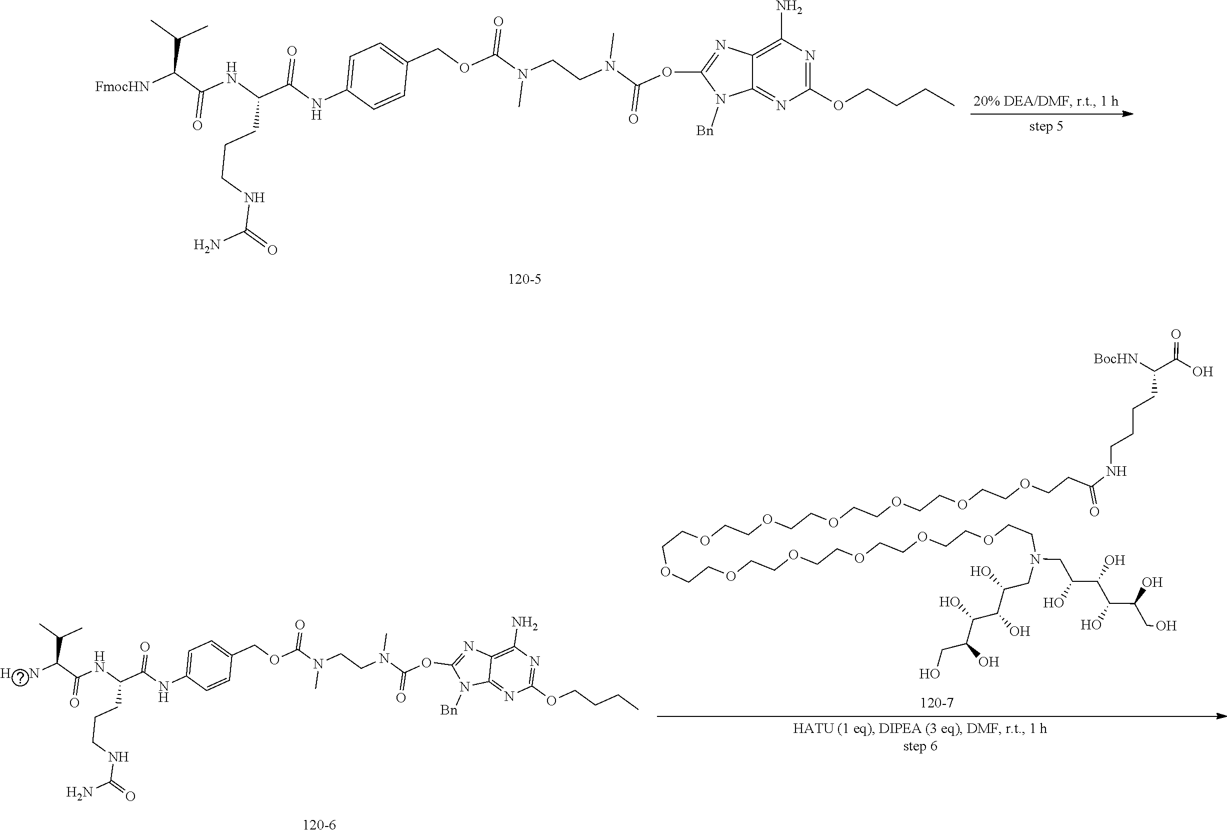

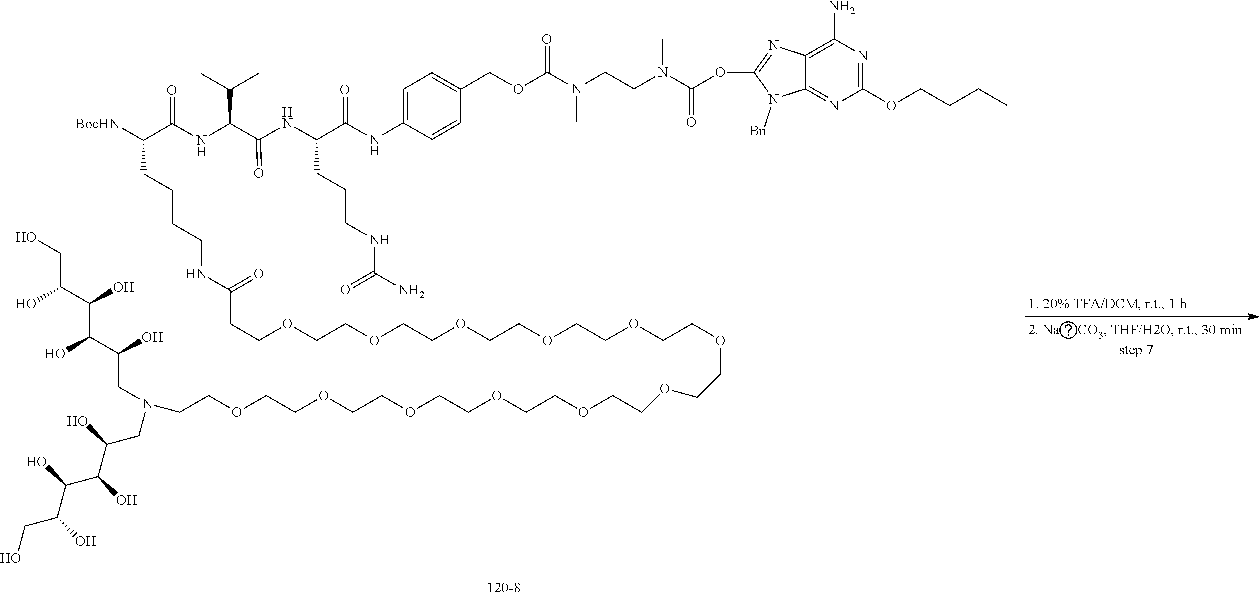

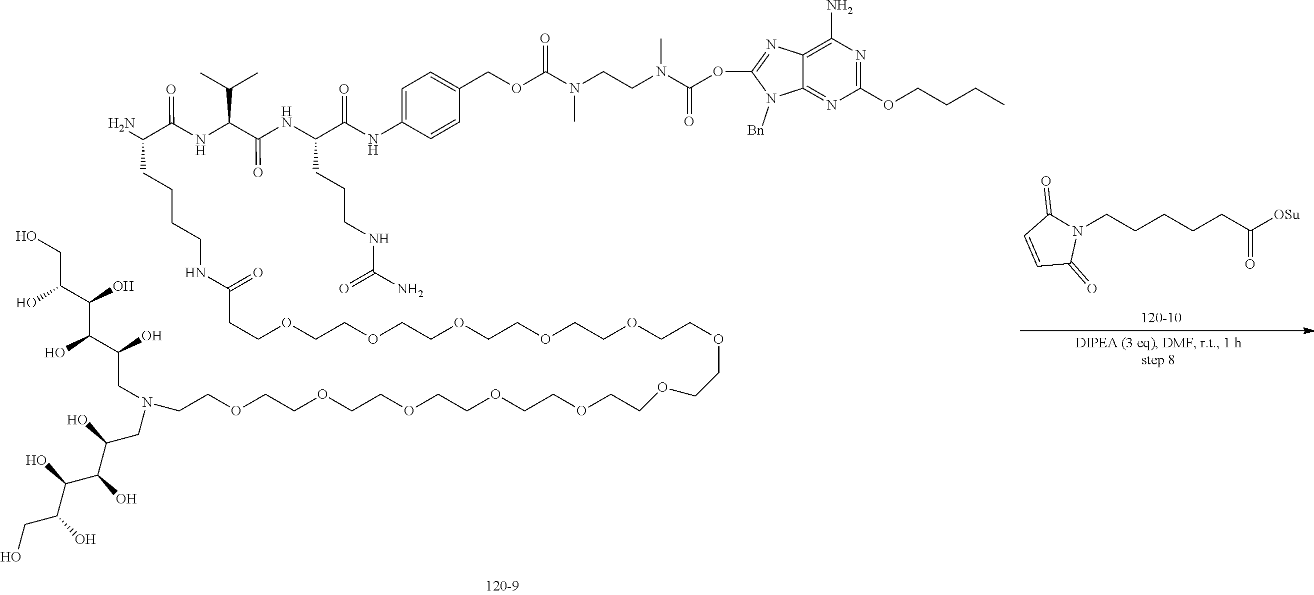

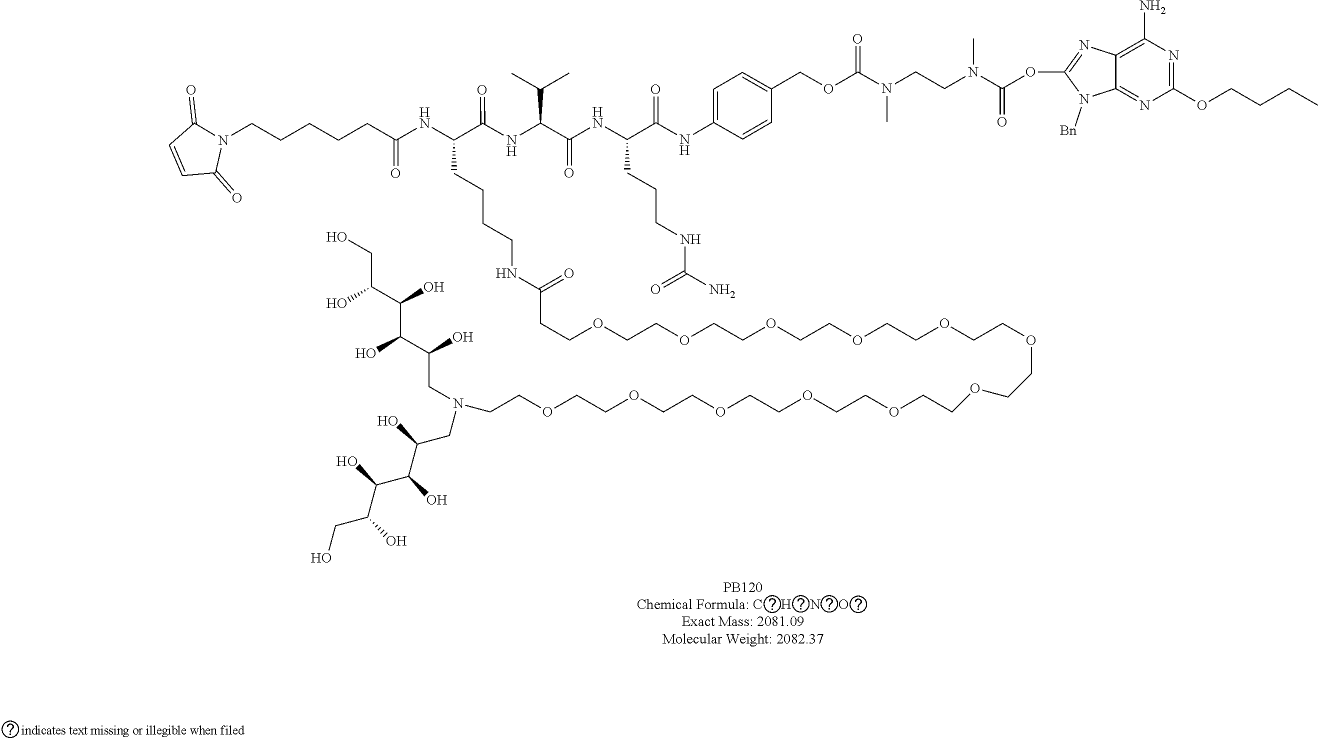

A Drug-Linker containing a PEG unit and a cleavable linker attached to T7-1 (PB120) was prepared as follows:

### Step 1

A solution of T7-1 (3 g, 9.574 mmol) and DIPEA (7.4 g, 57.444 mmol) in anhydrous DMF (30 mL) was stirred at room temperature for 5 min, then PNPC (8.7 g, 28.722 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (50 ml) at 5° C. for 30 minutes. The solution was filtered and the filter cake was washed with acetonitrile to yield compound 120-1 (4.1 g, 8.569 mmol, 89.52%) as a white solid. purity=90%-95%.

### Step 2

A solution of compound 120-1 (4.1 g, 8.569 mmol), N-Boc-N,N′-dimethylethylenediamine (1.61 g, 8.569 mmol) and HOBT (1.16 g, 8.569 mmol) in anhydrous DMF (10 mL) was stirred at room temperature, then DIPEA (3.3 g, 25.707 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 120-2 (2.3 g, 4.359 mmol, 50.88%) as a white solid. purity=90%-95%.

### Step 3

A solution of compound 120-2 (2.3 g, 4.359 mmol) and TFA (4 mL) in anhydrous DCM (16 mL) was stirred at room temperature for 1 hr until LCMS indicated all starting amine was consumed and the desired product was detected. Then the solution was concentrated to dryness to yield compound 120-3 (1.8 g, 4.210 mmol, 96.77%) as a yellow oil, which was used as such in the next step. purity=85%-90%.

### Step 4

A solution of compound 120-3 (1.8 g, 4.210 mmol), compound 120-4 (3.2 g, 4.210 mmol) and HOBT (0.57 g, 4.210 mmol) in anhydrous DMF (15 mL) was stirred at room temperature, then DIPEA (1.6 g, 12.630 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 120-5 (270 mg, 0.256 mmol, 6.14%) as a white solid. purity=85%-90%.

### Step 5

A solution of compound 120-5 (270 mg, 0.256 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 120-6 (210 mg, 0.252 mmol, 98.59%) as a colorless oil, which was used as such in the next step. purity=85%-90%.

### Step 6

A solution of compound 120-7 (296 mg, 0.252 mmol), compound 120-6 (210 mg, 0.252 mmol) and HATU (95.9 mg, 0.252 mmol) in anhydrous DMF (5 mL) was stirred at room temperature for 5 min, then DIPEA (97.8 mg, 0.756 mmol) was added. The resulting solution was stirred for another 1 hr at r.t. until LCMS indicated complete reaction. The reaction solution was purified directly by reverse phase liquid chromatography to yield compound 120-8 (120 mg, 0.060 mmol, 23.95%) as a white solid. purity=90%-95%.

### Step 7

A solution of compound 120-8 (120 mg, 0.060 mmol) and TFA (1 mL) in anhydrous DCM (4 mL) was stirred at room temperature for 1 hr until LCMS indicated all starting amine was consumed and the desired product (m/z=630=1889/3+H) along with sugar-esterificated product (TFA was condensed with hydroxy group in sugar unit, mono-ester with m/z=(1889+96)/3+H=662) were formed. The completed reaction solution was condensed to dryness and then redissolved in THF (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 120-9 (45 mg, 0.024 mmol, 39.47%) as a white solid. purity=90%-95%.

### Step 8

A solution of compound 120-9 (45 mg, 0.024 mmol) and DIPEA (9.2 mg, 0.072 mmol) in anhydrous DMF (2 mL) was stirred at room temperature for 5 min, then a solution of compound 120-10 (11.0 mg, 0.036 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB120 (15 mg, 0.007 mmol, 30.24%) as a white solid. LCMS, m/z=1042.19 (M/2+H)⁺. Drug linker PB120 can be used to make a conjugate such as PA120.

### Example 52: Preparation of the Conjugate (PA003) of Drug-Linker PB003 and 2E7 Antibody

A solution of 10 mg/mL of 2E7 antibody in pH 7.1 PB 5 mM EDTA buffer is reduced by 10 mM TCEP at 25° C. for 120 minutes. 6.5 eq of 5 mM PB03 in DMA is added to the reduced antibody solution, and the resulting mixture is stirred at 25° C. for 120 min. The ADC is purified with PD-10 column. The ADC is named PA003.

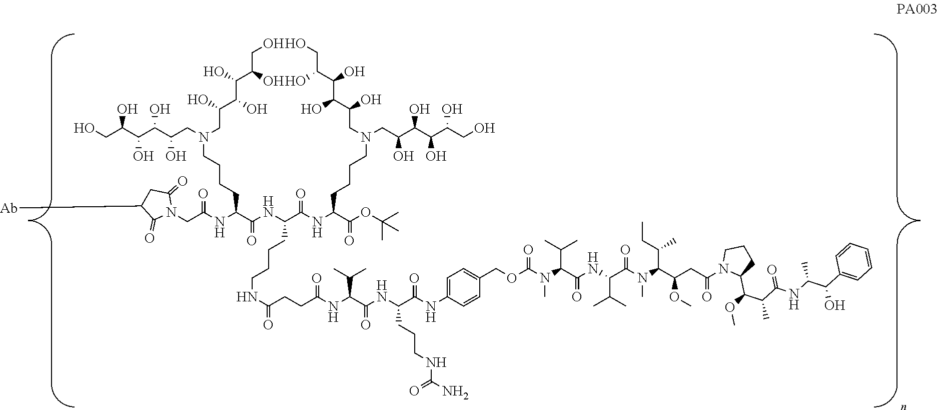

### Example 53: Preparation of the Conjugate (PA004) of Drug-Linker PB004 and 2E7 Antibody

A solution of 10 mg/mL of 2E7 antibody in pH 7.1 PB 5 mM EDTA buffer is reduced by 10 mM TCEP at 25° C. for 120 minutes. 6.5 eq of 5 mM PB004 in DMA is added to the reduced antibody solution, and the resulting mixture is stirred at 25° C. for 120 min. The ADC is purified with PD-10 column. The ADC is named PA004.

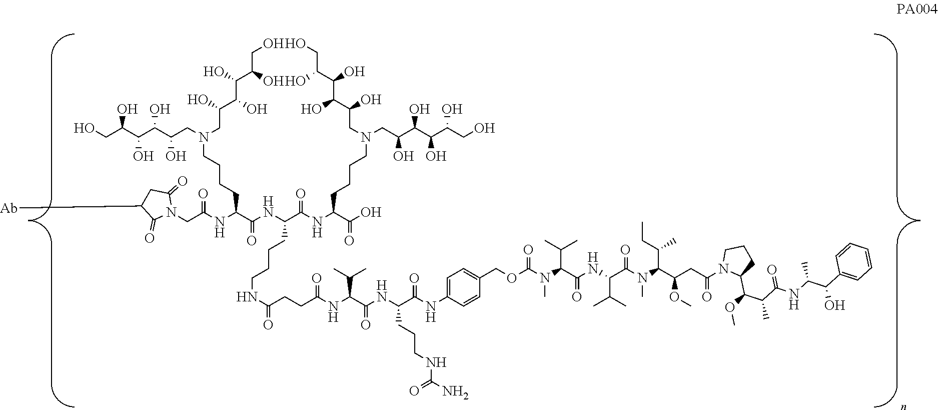

### Example 54: Preparation of the Conjugate (PA008) of Drug-Linker PB008 and 2E7 Antibody

A solution of 10 mg/mL 2E7 antibody in pH 7.1 PB 5 mM EDTA buffer is reduced by 10 mM TCEP at 25° C. for 120 minutes. 6.5 eq of 5 mM PB08 in DMA is added to the reduced antibody solution, and the resulting mixture is stirred at 25° C. for 120 min. The ADC is purified with PD-10 column. The ADC is named PA008.

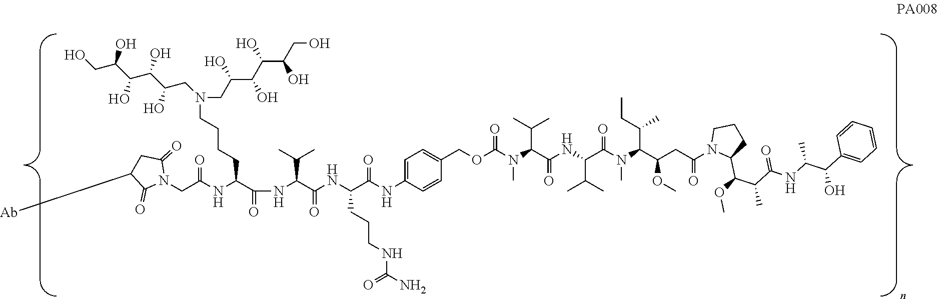

### Example 55: Preparation of the Conjugate (PA038) of Drug-Linker PB0038 and 2E7 Antibody

A solution of 10 mg/mL 2E7 antibody in pH 7.1 PB 5 mM EDTA buffer was reduced by 10 mM TCEP at 25° C. for 120 minutes. TCEP was removed with PD-10 column. 15 eq of 5 mM PB038 in DMA was added to the reduced antibody solution, and the resulting mixture was stirred at 25° C. for 120 min. The ADC was purified with PD-10 column. The ADC is named PA038 or 2E7-LD038.

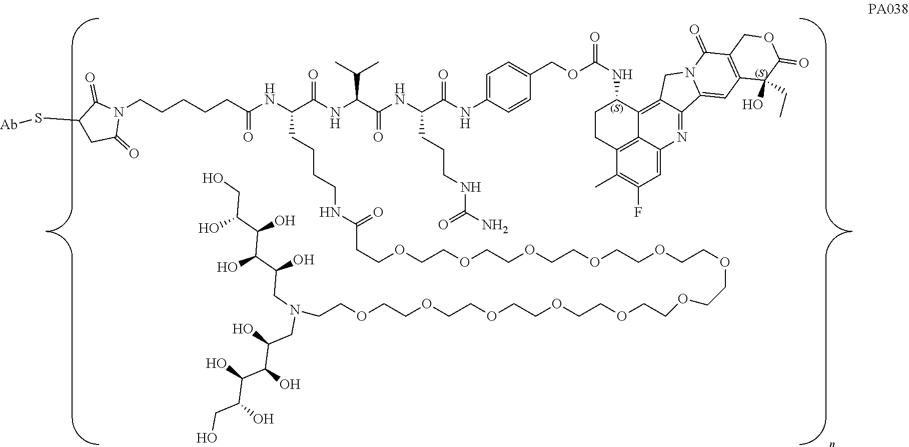

ADC PA0038 was analyzed by HIC-HPLC and SEC-HPLC.

### Example 56: Preparation of the Conjugate (PA039) of Drug-Linker PB039 and 2E7 Antibody

A solution of 10 mg/mL 2E7 antibody in pH 7.1 PB 5 mM EDTA buffer was reduced by 10 mM TCEP at 25° C. for 120 minutes. TCEP was removed with PD-10 column. 15 eq of 5 mM PB039 in DMA was added to the reduced antibody solution and the resulting mixture was stirred at 25° C. for 120 min. The ADC was purified with PD-10 column. The ADC is named PA039 or 2E7-LD039.

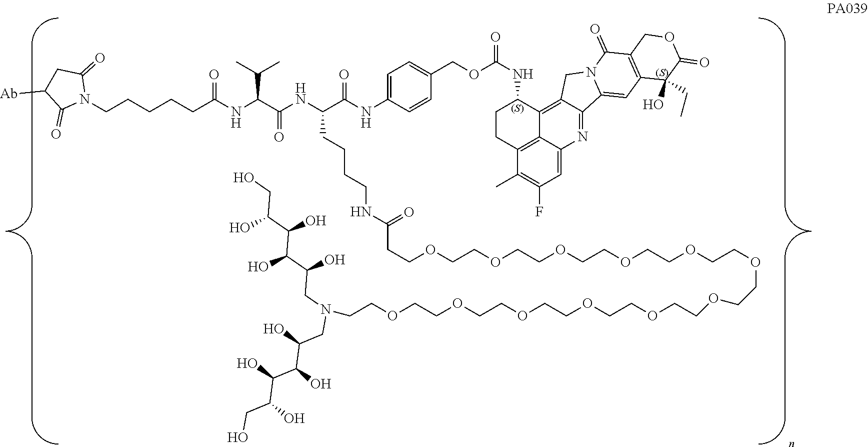

ADC PA0039 was analyzed by HIC-HPLC and SEC-HPLC.

### Example 57: Preparation of the Conjugate (PA040) of Drug-Linker PB040 and 2E7 Antibody

A solution of 10 mg/mL 2E7 antibody in pH 7.1 PB 5 mM EDTA buffer is reduced by 10 mM TCEP at 25° C. for 120 minutes. TCEP is removed with a PD-10 column. 15 eq of 5 mM PB040 in DMA is added to the reduced antibody solution, and the resulting mixture is stirred at 25° C. for 120 min. The ADC is purified with PD-10 column. The ADC is named PA040.

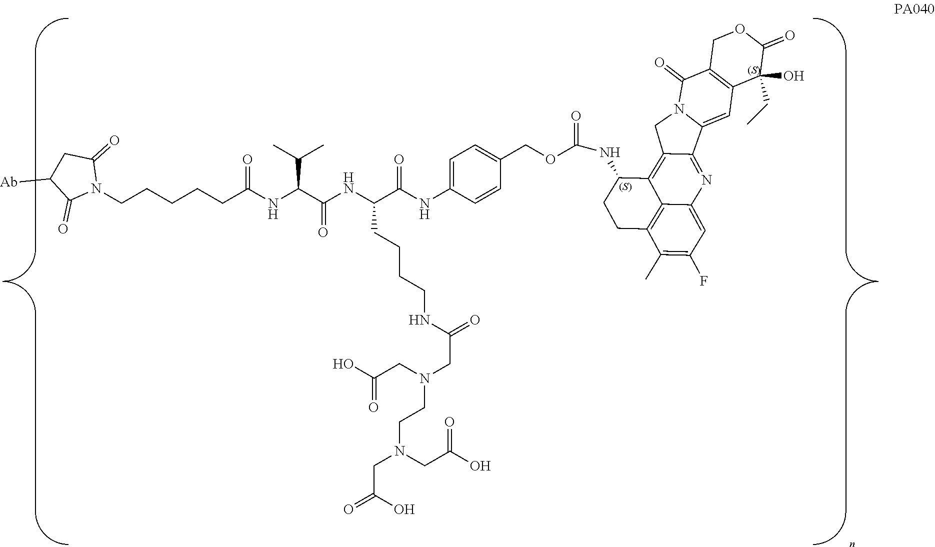

### Example 58: Preparation of the Conjugate (PA082) of Drug-Linker PB082 and 2E7 Antibody

A solution of 10 mg/mL 2E7 antibody in pH7.1 PB 5 mM EDTA Buffer is reduced by 10 mM TCEP at 25° C. for 120 minutes. TCEP is removed with a PD-10 column. 15 eq of 5 mM PB082 in DMA is added to the reduced antibody solution, and the resulting mixture is stirred at 25° C. for 120 min. The ADC is purified with PD-10 column. The ADC is named PA082.

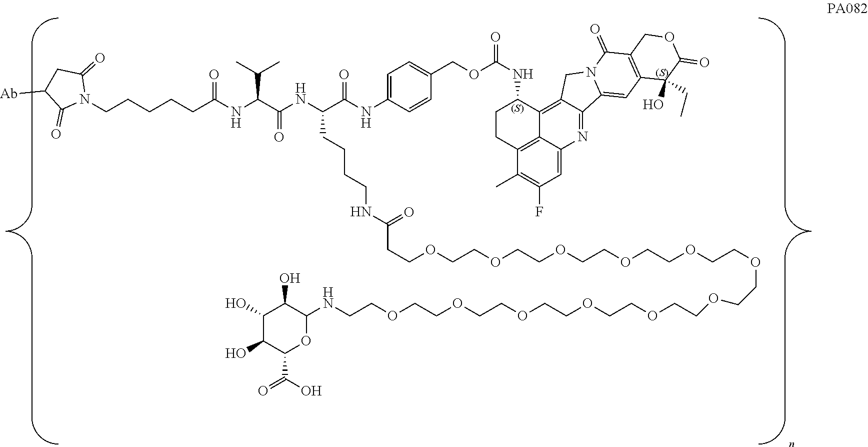

### Example 59: Preparation of the Conjugate (PA083) of Drug-Linker PB083 and 2E7 Antibody

A solution of 10 mg/mL 2E7 antibody in pH 7.1 PB 5 mM EDTA buffer is reduced by 10 mM TCEP at 25° C. for 120 minutes. TCEP is removed with a PD-10 column. 15 eq of 5 mM PB083 in DMA is added to the reduced antibody solution, and the resulting mixture is stirred at 25° C. for 120 min. The ADC is purified with a PD-10 column. The ADC is named PA083.

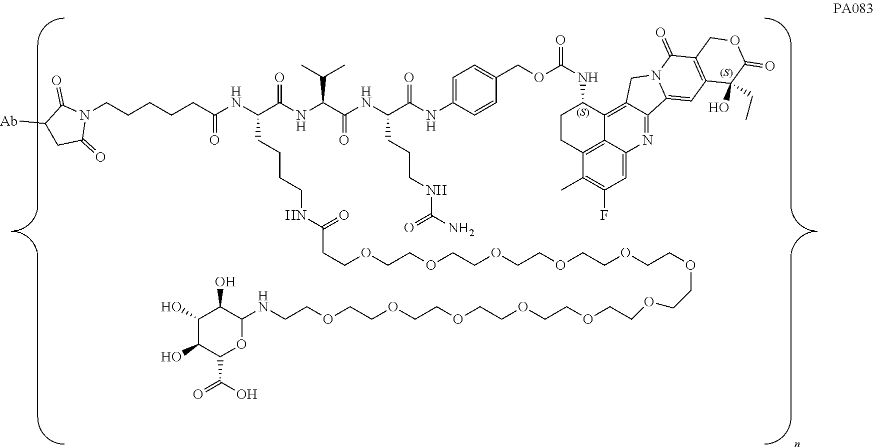

### Example 60: Stability Testing of ADC 2E7-LD038 (PA038)

When stored at 37° C. for up to 15 days or undergoing 5 cycles of freeze-thaw, ADC PA038 appeared to be stable by both HIC and SEC. No significant increase in aggregation was observed over time at 37° C. or multiple freeze-thaw cycles.

### Example 61: Retention Times Measured By HIC-HPLC

At DAR8 the relative retention times of ADCs PA038 (2E7-LD038) and PA039 (2E7-LD039) were shorter (11.9 minutes) than that of the DAR2 species of the corresponding MC-VC-PAB-MMAE-based ADC, 2E7-vedotin.

HIC-HPLC conditions: Column: TSKgel Butyl-NPR, 2.5 m, 4.6 mm×100 mm (PN: 42168); Column temperature: 30° C.; UV: 280 nm; Mobile phase: A: 50 mM PB, 1.5M (NH₄)₂SO₄, pH=7.0; B: 50 mM PB (pH=7.0):IPA=75:25 (v:v) Flow rate: 0.5 mL/min; Gradient elution: 0 min→20 min(0% B→100% B), 20 min→21 min(100% B→0% B), 21 min→32 min(0% B).

### Example 62: Generation of Human Antibodies Against Human CD70

Anti-CD70 antibodies with higher affinity and better characteristics were generated by random mutation of the CDR regions of the antibody heavy and light chain of parent antibody 69A7 (see U.S. Pat. No. 8,124,738B2). The heavy and light variable region amino acid sequences of 69A7 are set forth in SEQ ID NOs: 1 and SEQ 2, respectively. The HCDR and LCDR amino acid sequences are set forth in SEQ ID NOs: 21 to 26.

### CDR Scan Library Construction

Random mutations were introduced into each of the 6 CDRs of parent antibody 69A7 (3 HCDR+3 LCDR) (Chothia numbering convention) by PCR based mutagenesis with degenerative primers. The spliced PCR products were used for the library construction following a standard protocol.

The CDR library size was about 2 billion (2×10⁹) based on serial-dilution titration. Random colonies were picked for sequencing. The alignment of some random sequences from the un-selected library results showed that the random mutation library had good sequence diversity (data not shown). A phagemid library was rescued and used in the following panning procedure.

### Panning and Screening

The following standard protocol was followed for library panning. Immune tubes were coated with 0.5 ml of CD70 antigen at indicated concentration (see panning summary, Table 1), and placed in a refrigerator overnight. The tube was washed once with PBS, blocked with 1% BSA/PBS and placed at RT (room temperature) for 1 hour. The tube was incubated with the library phage sample at indicated amount (CFU, see the panning summary, Table 1) at RT for 1 hour. The tube was washed for 10 times with PBST buffer. To elute the bound phage, 0.5 ml of 100 mM TEA (triethylamine) was added and incubated at RT for 2 mins. The eluate was then transferred to a new tube and neutralized immediately by adding 0.25 ml of 1.0M Tris-HCL, pH 8.0, and mixing. The eluant (0.75 ml) was added into 10 ml of exponentially growing E. coli TG1 (OD600˜0.5), mixed well and incubated without shaking at 37° C. (water bath) for 30 min. 10-fold dilutions of the culture were made in 2×TY media and 10 μL of each dilution was plated on TYE/amp/glu plates, incubated at 30° C. overnight. The next day, the colony number for each dilution was counted, and the CFU (colony form unit) for the panning output was calculated. The remaining culture was centrifuged at 2,800 g for 15 mins, resuspend in 0.5 ml of 2×TY media, plated on two 150 mm TYE/amp/glu plates, and incubated at 30° C. overnight. The next day, 3 ml of 2×TY/amp/glu media was added to each plate and the bacteria were scraped from the plate with a cell spreader. Glycerol stocks was made by mixing 1.5 ml of bacteria and 0.5 ml of 80% glycerol, placed the stock at −80° C.

To prepare phage particles for the next round of selection, the glycerol stocks were inoculated into 40 ml of 2×TY/amp/glu media, starting at OD600˜0.01-0.05. The culture was grown at 37° C. with shaking (300 rpm) until the OD600 reached 0.4-0.6. The culture was infected by adding helper phage CM13 to the culture at a helper phage:bacteria ratio of 5-10:1. The bacterial culture was incubated at 37° C. for 30 minutes standing in a water bath with occasional mixing followed by shaking at 37° C. for 30 minutes. The bacterial culture was centrifuged at 3000 rpm for 20 minutes, and the supernatant was removed. The pellet was resuspended in 100 mL of 2×TY/amp/kan and grown with shaking at 30° C. overnight. The culture was then harvested by centrifuging at 6,000 g for 30 mins. The phage particles were precipitated by adding 1/5 volume of PEG solution into the supernatant followed by 1 h incubation on ice, and then centrifuging at 4,000 g for 20 mins at 4° C. The supernatant was thoroughly removed. The phage pellet was resuspended in 1-2 ml of cold PBS. Any residual bacteria were removed by micro-centrifugation at top speed for 5 mins at 4° C. The prepared phage were either used immediately for selection or stored at −80° C. in aliquots with 10% glycerol. The titer of the phage preparation was determined by infecting 100 μL of exponentially growing E. coli TG1 with 10-fold dilution of the phage solution (in 2×TY, down to 1011). The selection step was repeated, starting with step 1, for a total round of 3 rounds.

As discussed above, 3 rounds of panning were performed. The concentrations of the washing buffer PBS-Tween20 in the 2^{nd} and 3rd rounds was gradually increased with 0.2%, 0.3%, respectively, and the coating antigen in the 2^{nd} and 3rd rounds was gradually decreased to 4 μg and 2 ug, respectively. After 3 rounds of screening, the target positive enrichment rate reached 6.9×10⁵ (690 thousand) with a significant difference from the blank control, as shown in Table 1.

### ELISA Assays on Purified Phage Samples.

A sterile 96-well round-bottom microtiter plate was filled with 1001/well of 2×YT-2% Glucose. Single TG1 colonies were picked up from selection plates of the 3^{rd} (last) enriched round using sterile pipette tips and used to inoculate one well/colony. The plate was sealed with a breathable membrane and incubated at 30° C. overnight while shaking. This plate was designated the master plate. On the next day, an aliquot of the cultures was transferred to a new deep-well induction plate containing 400 μl/well 2×YT-0.1% Glucose. About 101/well from the master plate was pipetted using a multichannel pipette to the new induction plate. The induction plate was incubated for about 2-4 hrs in the phage orbital shaker until the bacteria reached log-phase (37° C., 200 rpm). IPTG was added to each well at a final concentration of 0.2 mM and the plate was cultured overnight at 30° C. with shaking at 200 rpm. The next day, the induction plate was spun at 3,500 rpm for 10 min. The supernatant was used in the following scFv ELISA. (The plates were optionally placed at 4° C. for temporary storage; the supernatant could be used within 2 weeks.)

The binding of the phage to CD70 antigen was tested in a phage ELISA. Briefly, the antigen CD70 (ACRO, CDL-H5246) was diluted to 5 g/ml and coated onto a microtiter plate overnight using 100 L/well. The next day the plate was washed 2× with PBST (0.1% Tween20 in PBS) using 200 L/well. The blocking buffer (2% milk in PBST) was added using 200 L/well. The plate was placed at RT for 1 hr. The plate was washed two times with PBST. IPTG-induced culture supernatants were added to each well using 50 μL/well and placed at RT for 1 hr. The plate was washed 3-4 times with PBST. Anti-human-HRP was diluted at 1:5,000 into PBST using 50 μL/well. The plate was incubated at RT for 20 min. The plate was then washed again for 5 times with PBST. Fresh developing solution (10 ml of Developing buffer, 13.3 μL Amplex Red (5 mg/ml in DMSO), 3.3 μL H₂O₂) was prepared, added using 50 μL/well, and the color was developed at RT for 1-60 min. The plate was read at Ex=530 nm, Em=590 nm and cutoff=570 nm.

TABLE 1
Summary of Panning Procedure

| | Round 1 | Round 2 | Round 3 |
|---|---|---|---|
| Input (AMX scFv) | 7.6 × 10¹¹ | 9.6 × 10¹⁰ | 4.8 × 10¹⁰ |
| Output | 1.8 × 10⁶ | 2.6 × 10⁶ | 7.0 × 10⁴ |
| Enrichment | 4.2 × 10⁵ | 3.7 × 10⁴ | 6.9 × 10⁵ |
| CD70 antigen | 6 ug | 2 ug | 1 ug |

Using the scFv Elisa procedure described above, two 96-well plates of single colonies were picked, cultured, and induced for scFv expression. The scFv supernatant was used in the screening assay.

20 clones with positive signals were sequenced. The sequences of 5 selected clones are shown in Table 2. The HCDR and LCDR in each variable region are marked in bold. These clones have at least two more amino acid substitutions in HCDR3/LCDR3 (SEQ ID NOs: 13 to SEQ18) for each candidate antibody compared with the parent antibody 69A7. The 5 unique sequences, 2A4, 1H8, 2E7, 2D2 and 1A4, were picked for further analysis. One new VH/VL combination was prepared; the VH/VL pair of 2A4P0L1 was derived from the VH of 2A4 and the VL of 2D2.

TABLE 2
Variable region sequence of anti-CD70 antibodies

| | Seq. | |
|---|---|---|
| Clone | VH | VL |
| 2A4 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPED |
| | QFSLKLRSVTTADTAVYYCARGDGDYGGNVFPYWGQG | FAVYYCQQRSNWPLTFGGGTKVEIK (SEQ 4) |
| | TLVTVSS (SEQ 3) | |
| 1H8 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPED |
| | QFSLKLRSVTTADTAVYYCARGDGDFMGVCFDYWGQG | FAVYYCQQRSNWPLTFGGGTKVEIK (SEQ 6) |
| | TLVTVSS (SEQ 5) | |
| 2E7 | QVQLQESGPGLVKPSETLSLTCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPED |
| | QFSLKLRSVTTADTAVYYCARGDGDFLGVCFDYWGQG | FAVYYCQQRSNWPLTFGGGTKVEIK (SEQ 8) |
| | TLVTVSS (SEQ 7) | |
| 2D2 | QVQLQESGPGLVKPSETLSL TCTVSGGSVSSDYYYWSWI | EIVLTQSPATLSLSPGERATLSCRASQSVSSYLAWYQQKPG |
| | RQPPGKGLEWLGYIYYSGSTNYNPSLKSRVTISVDTSKN | QAPRLLIFDASNRATGIPARFSGSGSGTDFTLTISSLEPED |
| | QFSLKLRSVTTADTAVYYCARGDGDYGGNCFDYWGQG | FAVYYCQQRLKFPLTFGGGTKVEIK (SEQ 10) |
| | TLVTVSS (SEQ 9) | |
| 1A4 | QVQLQESGPGLVKPSETLSLTCTVSGGSVYSGYYYWSWI | EIVLTQSPATLSLSPGERATLSCRASQSVSSYLAWYQQKPG |
| | RQPPGKGLEWLGYFSLSGSTNYNPSLKSRVTISVDTSKN | QAPRLLIFDASNRATGIPARFSGSGSGTDFTLTISSLEPED |
| | QFSLKLRSVTTADTAVYYCARGDGDYGGNCFDYWGQG | FAVYYCQQRSNWPLTFGGGTKVEIK (SEQ 12) |
| | TLVTVSS (SEQ 11) | |

### Example 63: Production of Anti-CD70 Antibodies

Full IgG anti-CD70 lead antibodies (1A4, 2A4, 1H8, 2D2, 2E7 and 2A4P0L1) and the reference parent antibody (69A7) and another reference antibody, 1F6 (Vorsetuzumab, see U.S. Pat. No. 7,491,390), were constructed from the 6 leads and two controls so as to have their human heavy and light variable regions connected to the human constant regions IgG1 and Kappa, respectively. (The VH and VL sequences of 1F6 antibody are shown in SEQ ID NOs: 19 and 20, respectively.) Briefly, the Kozak consensus sequence “GCCGCCACC” (SEQ ID NO:31) and the signal peptide “MGWSCIILFLVATATGVHS” (SEQ ID NO: 32) were inserted at the 5′ terminal of the gene construct for adequate translation and antibody secretion. The final DNA coding sequence of heavy and light chains was optimized and synthesized and constructed in the vector pcDNA3.4.

The resulting plasmids were transiently transfected into ExpiCHO-S cells using a ExpiCHO™ Expression System (Thermo, ExpiFectamine™ CHO Transfection Kit, Cat #A29129) based on a standard ExpiFectamine CHO Transfection procedure (Gibco, A29129) in spinner flasks. The suspensions of transient transfections were incubated for 10 days and then the cleared supernatants were purified using Protein A columns.

Antibodies were purified from cleared cell culture supernatants using Protein A chromatography (Protein A resin slurry, 4.5 mL, Bogen, Cat #18-0010-02). Briefly, supernatants were prepared for affinity chromatography and loaded onto the columns and allowed to flow completely through the resin. The columns were washed with binding buffer containing 0.15M NaCl and 0.2M PB, PH7.0. Antibody was eluted with elution buffer containing 0.15M NaCl, 0.1M Glycine and 0.2M PB, PH 3.0. Fractions were collected and neutralized by the addition of 1/10 volume of 1M Tris, PH9.0. The fractions were dialyzed for 2 hours against 1×PBS. Purified antibody was quantified by absorbance at A280. Samples from each step of the protein A chromatography were applied onto 12% SDS-PAGE gels for reduced and non-reduced electrophoresis. The hydrophobicity was assessed with hydrophobic interaction chromatography (HIC) on a TSK gel Butyl-NPR, 4.6×100 mm with Butyl-NPR (Tosoh corporation) using a Waters HPLC 2695 system.

The expression levels following purification are shown in Table 3. Antibody clone 2A4 had a higher expression level; clone 1A4, 2D2 and 2E7 had medium expression levels and clone 2A4P0L1 and 1H8 had lower expression levels.

The hydrophobicity analyses are shown in Table 4. Clone 2D2 and 2A4 had the similar hydrophobicity with the parent antibody, 69A7; clone 2A4P0L1, 1H8 and 2E7 were slightly more hydrophobic than the parent antibody 69A7.

TABLE 3
Comparison of antibodies expression levels

| | Trans Vol | Conc. | Vol. | Quantity |
|---|---|---|---|---|
| Sample Name | (ml) | (mg/ml) | (ml) | (mg) |
| 1A4 | 60 | 1.965 | 6.5 | 12.77 |
| 2A4 | 60 | 3.356 | 6.5 | 21.81 |
| 2A4P0L1 | 60 | 0.663 | 6.5 | 4.30 |
| 1H8 | 60 | 0.541 | 6.5 | 3.52 |
| 2D2 | 60 | 1.993 | 6.5 | 12.95 |
| 2E7 | 60 | 1.782 | 6.5 | 11.58 |
| 69A7 | 60 | 2.78 | 10.5 | 29.19 |

TABLE 4
Hydrophobicity of anti-CD70 antibodies

| Sample name | Retention time (min) |
|---|---|
| 2A4P0L1 | 9.56 |
| 2A4 | 9.06 |
| 2D2 | 8.81 |
| 1H8 | 10.24 |
| 2E7 | 10.19 |
| 69A7 | 8.83 |

### Example 64: In Vitro Binding of 2E7 Antibody and 2E7-LD038 Conjugate to Recombinant Human, Cynomolgus Monkey, Rat, and Mouse CD70 Protein, Via Biolayer Interferometry (BLI)

Recombinant proteins consisting of the CD70 extra-cellular domain (ECD) linked to His tag were either purchased (from KACTUS or ACRO biosystems) or synthesized in house. 2E7 antibody or 2E7-LD038 conjugate (20 nM) was immobilized on anti-human IgG Fc biosensor tips (Fortebio). Binding assays using varying concentration from 200 nM down to 3.13 nM of recombinant protein in solution were performed using Octet RED (Fortebio). Association time was set at 180 seconds and dissociation time was set at 300 seconds. Binding affinity was calculated using ForteBio Data Acquisition 6.3 software (ForteBio). Affinity was derived by fitting the kinetic data to a 1:1 Langmuir binding model utilizing global fitting algorithms.

2E7 antibody and 2E7-LD038 conjugate showed high binding affinity to human CD70 and cyno CD70 with the equilibrium dissociation constant (KD) in the range of 0.1˜1 nM. 2E7 antibody and 2E7-LD038 conjugate displayed no cross-reactivity to rat and mouse CD70 (see Table 5).

TABLE 5
Affinity data of 2E7 antibody and 2E7-LD038 conjugate
to CD70 of different test species by BLI

| | | KD | ka | kdis | Full |
|---|---|---|---|---|---|
| Antigen | Analyte | (M) | (1/Ms) | (1/s) | R\hat{ }2 |
| Human | 2E7 | 8.068E−10 | 3.143E05 | 2.536E−04 | 0.9956 |
| CD70 | | | | | |
| Human | 2E7- | 1.197E−10 | 4.556E05 | 5.454E−05 | 0.9965 |
| CD70 | LD038 | | | | |
| Cyno CD70 | 2E7 | 3.900E−10 | 6.178E05 | 2.409E−04 | 0.9943 |
| Cyno CD70 | 2E7- | 1.443E−10 | 6.516E05 | 9.403E−05 | 0.9951 |
| | LD038 | | | | |
| Rat CD70 | 2E7 | — | — | — | — |
| Rat CD70 | 2E7- | — | — | — | — |
| | LD038 | | | | |
| Mouse CD70 | 2E7 | — | — | — | — |
| Mouse CD70 | 2E7- | — | — | — | — |
| | LD038 | | | | |

—: Response below range of quantification

### Example 65: In Vitro Binding of 2E7 Antibody and 2E7-LD038 Conjugate to Human Tumor Cell Lines with or without CD70 Expression

Raji (ATCC, CCL-86), Caki-1 (Cobioer, CBP60448), MCF-7 (Cobioer, CBP60380) were obtained from Cobioer and 786-O (BNCC, BNCC338472) was obtained from BNCC. Cell lines were cultured in CO₂ thermostatic incubator at 37° C. with 5% CO₂ in standard conditions. Raji and 786-O were cultured with RPMI1640 (Gibco, cat #11875093) medium supplement of 10% FBS (Cellmax, cat #SA211.02), Caki-1 was cultured with MoCoy's 5A (Gibco, cat #16600082) supplement of 10% FBS, MCF-7 was cultured with MEM (Gibco, cat #11095-080) supplement of 10% FBS and 10 g/ml insulin (Solarbio, 18830-100 mg). Cells were sub-cultured every 1-2 days, digested with 0.25% trypsin and passaged at a suitable density. When the cells were in the logarithmic growth phase and the amount could meet the assay demand, cells were collected and counted for the subsequent cell-based assays.

The binding activity of 2E7 antibody or 2E7-LD038 ADC was evaluated by Flow cytometric analysis (Beckman, Cytoflex) with target-expressing cell lines, Raji, 786-O, Caki-1, and a cell line with negligible CD70 expression, MCF-7. 3×10⁵ cells were seeded per well at 96-well v-bottomed plate and incubated with 100 μl of 2E7 or 2E7-LD038 in serial dilutions. After 30 minutes of incubation at 4° C., cells were washed twice with PBS, stained with 100 μl of 1:200 diluted PE-conjugated anti human Fc in FACS buffer (1×PBS containing 1% BSA) and then incubated at 4° C. for 30 minutes. After that the cells were washed 2 times with PBS and analyzed by Flow cytometric analysis.

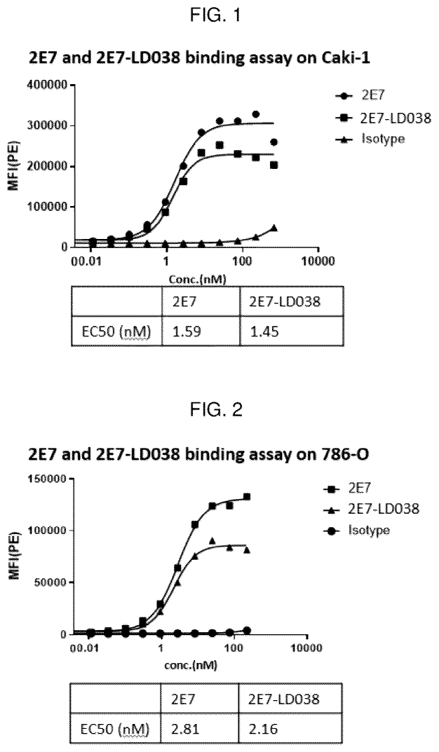

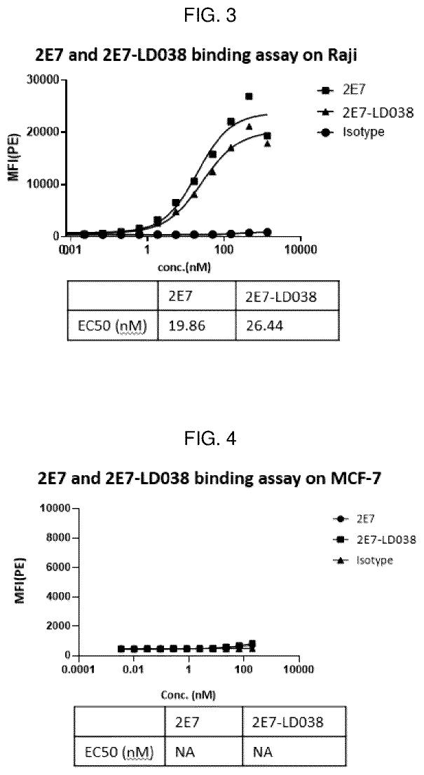

2E7 antibody and 2E7-LD038 conjugate demonstrated strong binding activity to human CD70-expressing cell lines with the EC50 in the range of 1-27 nM (see, FIGS. 1-4). The binding activity of 2E7 antibody was not compromised after conjugation. 2E7 antibody or 2E7-LD038 conjugate did not bind the cell line that has negligible CD70 expression, MCF-7.

### Example 66: In Vitro Blockade of CD27 Binding by 2E7 Antibody or 2E7-LD038 Conjugate Via FACS

CD70-positive cell lines 786-O, Raji and Caki-1 were mixed with varying concentrations from 333.5 nM down to 6 nM of 2E7-LD038 conjugate, or 2E7 antibody, or negative binding control (B12), and incubated at 4° C. for 30 minutes. Cells were washed twice with PBS and then incubated with 30 g/ml biotinylated human CD27 (the receptor for CD70) at 4° C. Staining was done using PE-conjugated streptavidin for 30 minutes and fluorescence was measured using flow cytometer.

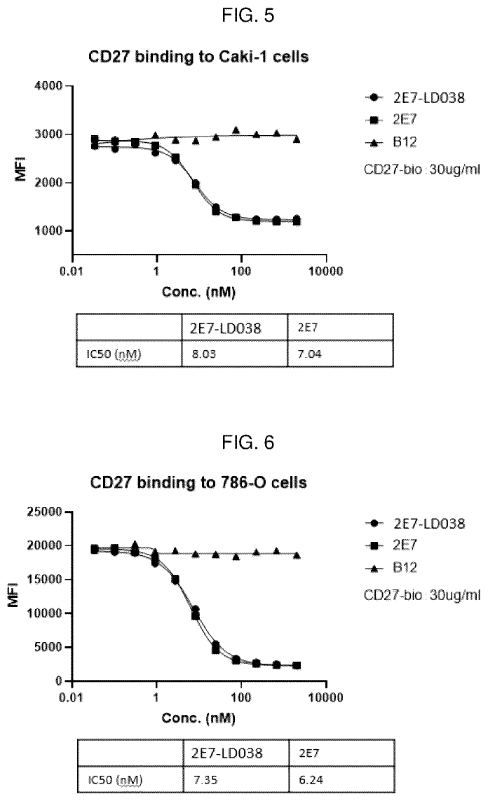

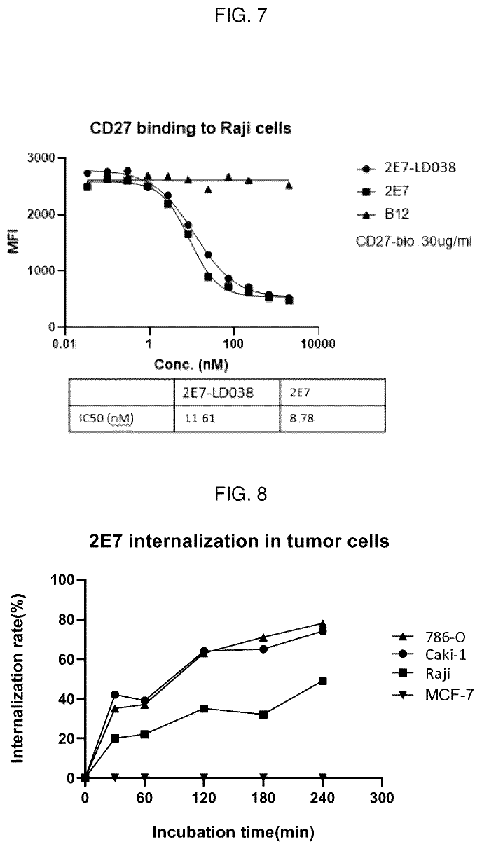

2E7 antibody and 2E7-LD038 conjugate, but not control antibody B12, showed strong blocking activity against the CD70-CD27 interaction on human CD70-expression cell lines with the IC50 in the range of 7-12 nM. The blocking activity of 2E7 antibody was not compromised after conjugation (see, FIGS. 5-7).

### Example 67: 2E7 Antibody and 2E7-LD038 Conjugate Internalization Using Additional Cell Lines (786-O, Caki-1, Raji, and MCF7) in Time Course

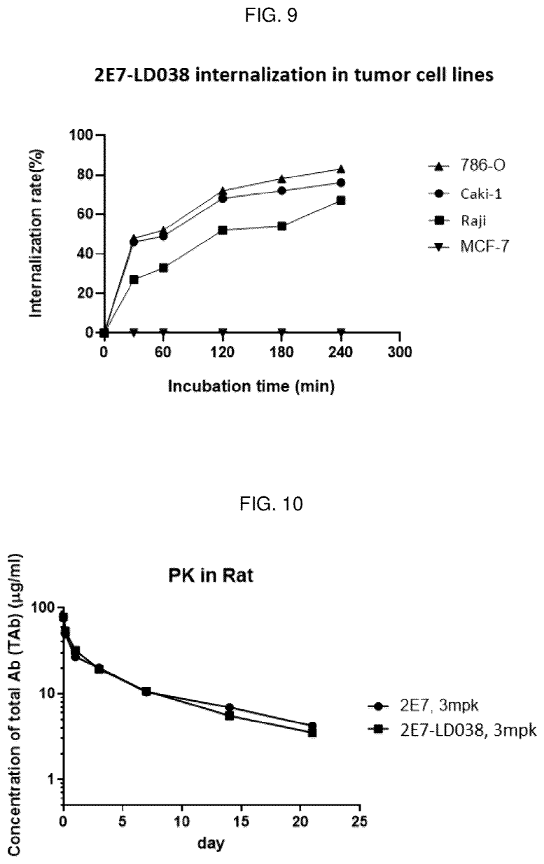

Four cell lines (786-O, Caki-1, Raji, and MCF-7) were utilized in the internalization assay. Target (CD70) copy numbers were determined via the QIFIKIT (DAKO, K0078). Briefly, cells were labeled with a primary mouse monoclonal antibody against CD70. Cells, set-up beads, and calibration beads (from the kit) were then labeled in parallel with a fluorescein-conjugated anti-mouse secondary antibody. The fluorescence is correlated with the number of bound primary antibody molecules on the cells and on the beads. Samples were subsequently analyzed on the flow cytometer and copy number determined based on the calibration curve (Table 6). For internalization assay, 3×10⁵ cells were incubated at 4° C. for 30 min with 10 μg/ml 2E7 antibody or 2E7-LD038 conjugate in FACS buffer (1×PBS containing 0.1% BSA). Cells were washed at 4° C. to remove unbound material and kept on ice or shifted to 37° C. for different lengths of time as needed. At progressive timepoints (0 hr, 0.5 hr, 1 hr, 2 hr, 3 hr, 4 hr) cells were stained with PE-conjugated anti-human Fc for 30 min at 4° C. and analyzed by flow cytometry. Internalization rate were calculated by subtracting the mean fluorescence intensity (MFI) of cell surface-bound antibody at 37° C. at each time point from the MFI of cell surface-bound antibody at 4° C. at time 0, then divided by the MFI of the cell surface-bound antibody at 4° C. at time 0. Both 2E7 antibody and 2E7-LD038 conjugate displayed rapid internalization on CD70-expressing cell lines (786-O, Caki-1, Raji), and no internalization on the CD70-negative cell line (MCF-7) (FIG. 8 and FIG. 9).

TABLE 6
Target (CD70) copy numbers in cell lines

| | | Copy number |
|---|---|---|
| Cell line | Tumor type | (×10³ per cell) |
| 786-O | renal | 149 |
| Caki-1 | renal | 119 |
| Raji | lymphoma | 62 |
| MCF-7 | breast | 3.8 |

### Example 68: PK of 2E7 Antibody and 2E7-LD038 Conjugate in Rat Model

2E7 antibody and 2E7-LD038 conjugate were administered via intravenous infusion at 3 mg/kg to male Sprague Dawley rats (n=3 per group). Orbital blood was sampled from each rat at various time points (10 min, 4 hr, 1 day, 3 days, 7 days, 14 days, and 21 days) post dosing. Total antibody (TAb), concentration of 2E7 antibody and 2E7-LD038 conjugate in plasma were detected by anti-idiotype (from Genscript) and calculated using Winnonlin 8.2 software. 2E7-LD038 conjugate was stable in circulation and displayed PK characteristics that are indistinguishable from the parental mAb (2E7) (FIG. 10).

### Example 69: 2E7 Conjugates: In Vitro Cytotoxicity

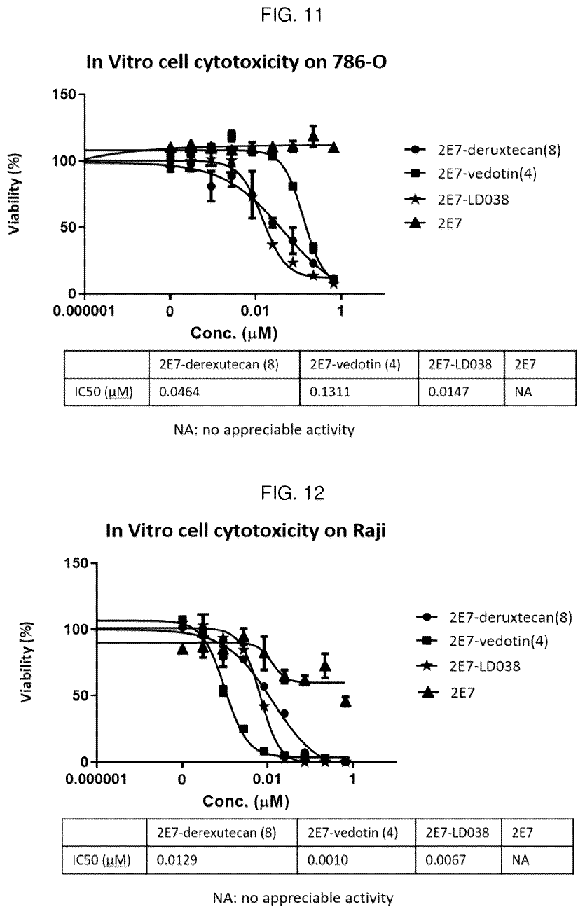

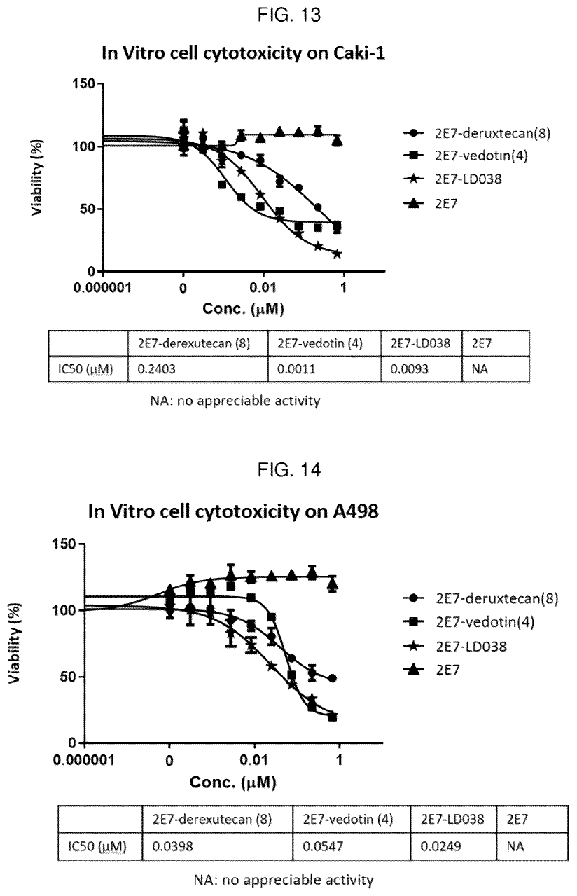

Two 2E7-conjugates were utilized in the study (Table 7). For preparation of 2E7-deruxtecan conjugate: 2 mL of 2E7 antibody (10 mg/mL) in 50 mM sodium phosphate buffer containing 5 mM EDTA (pH=6.9) was added to the aqueous solution of 10 mM TCEP HCl (Tris(2-carboxyethyl) phosphine HCl), at the molar ratio of 8.0 (TCEP to mAb). The reducing reaction proceeded for 2 hr at 25° C. Deruxtecan (mc-GGFG-DXd; dissolved in DMSO at a concentration of 20 mg/mL) was added to the reduced antibody at a molar ratio of 12 (deruxtecan/mAb). The coupling reaction was stirred for 8 hr at 25° C. The excess deruxtecan and the impurities were removed by ultrafiltration with 50 mM sodium phosphate buffer. The ADC was stored in 20 mM histidine buffer containing 6% sucrose and 0.02% (w/V) Tween 20 by UFDF. The purity measured by SEC-HPLC was 97.2% and DAR value measured by LC-MS was 7.5. For preparation of 2E7-vedotin conjugate, 2 mL of 2E7 antibody (10 mg/mL) in 50 mM sodium phosphate buffer containing 5 mM EDTA (pH=6.9) was added the aqueous solution of 10 mM TCEP HCl (Tris(2-carboxyethyl) phosphine HCl), at the molar ratio of 2.2 (TCEP to mAb). The reducing reaction proceeded for 2 hr at 25° C. Vedotin (mc-vc-PAB-MMAE; dissolved in DMSO at a concentration of 20 mg/mL) was added to reduced antibody at a molar ratio of 5.0 (vedotin/mAb). The coupling reaction was stirred for 2 hr at 25° C. The excess vedotin and the impurities were removed by ultrafiltration with 50 mM sodium phosphate buffer. The ADC was stored in 20 mM histidine buffer containing 6% sucrose and 0.02% (w/V) Tween 20 by UFDF. The purity measured by SEC-HPLC was 97.4% and DAR value measured by HIC-HPLC was 3.9. For the in vitro cytotoxicity study: one day prior to adding 2E7 conjugates, cells were harvested and plated into 96-well solid white flat bottom plates. The next day cells were exposed to the test article at a concentration range of 670 nM to 0.00067 nM. Plates were incubated at 37° C. for 96 h. After that, 40 μl Cell-tire Glo (CTG) per well was added to the plates with luciferase reading collected at 5 min after incubation, and analyzed by Microplate readers. All readings were normalized as percentage of viable cells in the untreated control wells and the IC50 values were calculated by Prism software. All three ADCs elicited cytotoxicitic effect in all 4 cell lines tested. 2E7-LD038 displayed comparable to higher potency relative to 2E7-deruxtecan (8-load). Activity of 2E7-vedotin (4-load) relative to 2E7-LD038 was variable, with the highest potency for 2E7-vedotin (4-load) observed in the Raji and Caki-1 cells. The parent antibody, 2E7, displayed no appreciable cytotoxic activity, consistent with expectations (FIGS. 11-14).

TABLE 7
2E7 conjugates utilized in the study

| Benchmarking ADC (DAR) | Linker-drug |
|---|---|
| 2E7-deruxtecan(8) | mc-GGFG-DXd |
| 2E7-vedotin(4) | mc-vc-PAB-MMAE |

### Example 70: In Vitro Cytotoxicity of 2E7-LD038 Conjugate in Human CD70-Expressing Tumor Cells

Raji (ATCC, CCL-86), Caki-1 (Cobioer, CBP60448), and 786-O (BNCC, BNCC338472) were cultured in CO₂ thermostatic incubator at 37° C. with 5% CO₂ in standard conditions. Cells were sub-cultured every 1-2 days, digested with 0.25% trypsin and passaged with a suitable density. When the cells were in the logarithmic growth phase and the amount meeting the assay's demand, cells were collected and plated onto 96-well solid white flat bottom plates for assay. One day later test articles (2E7 antibody or 2E7-LD038 conjugate ranging from 2 μM to 0.1 nM, or exatecan ranging from 20 μM to 0.03 nM) were added to the cells. Plates were then incubated at 37° C. for 96 h. Cell Titer Glo (CTG) (40 μl per well) was subsequently added to the plates. Luciferase reading was collected 5 min after and analyzed by Microplate readers. All readings were normalized as percentage of viable cells in the untreated control wells and the IC₅₀ values were calculated by Prism software.

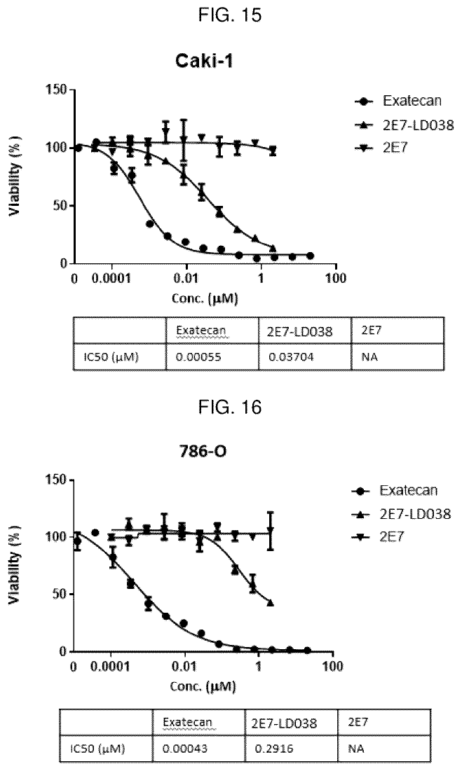

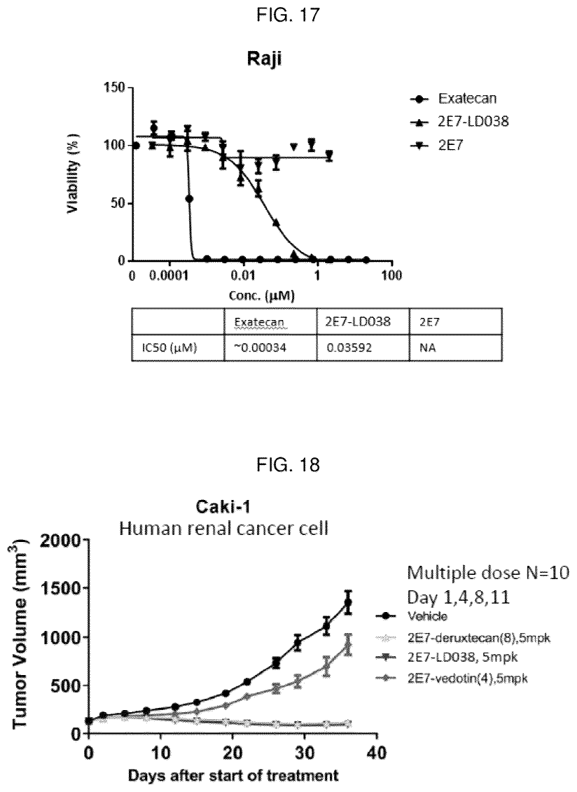

2E7-LD038 conjugate produced potent cytotoxic effects in Raji and Caki-1 cells (IC50=36, 37 nM, respectively), and was moderately cytotoxic on 786-O cells (IC50=292 nM) (see, FIGS. 15-17). Exatecan was cytotoxic on all cell lines whereas 2E7 antibody alone was inactive in the assay.

### Example 71: 2E7 Conjugates: In Vivo Efficacy in Cell Line-Derived Xenograft (CDX) Models

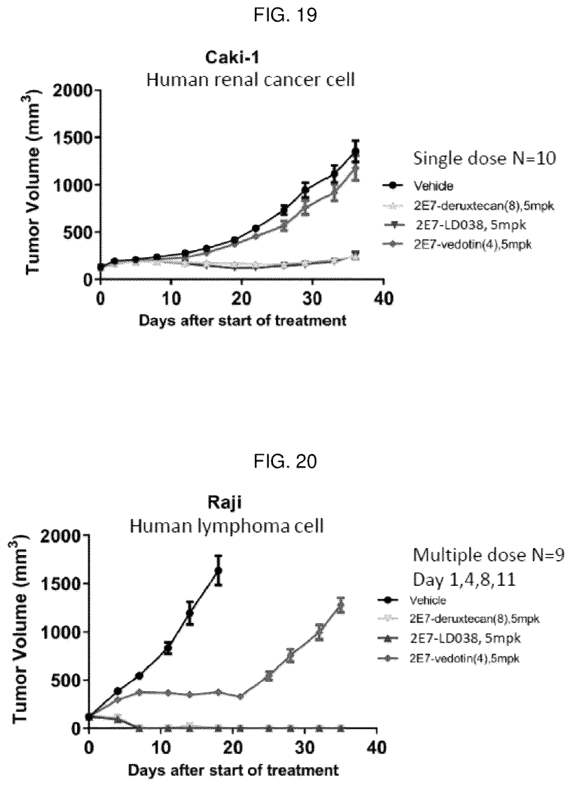

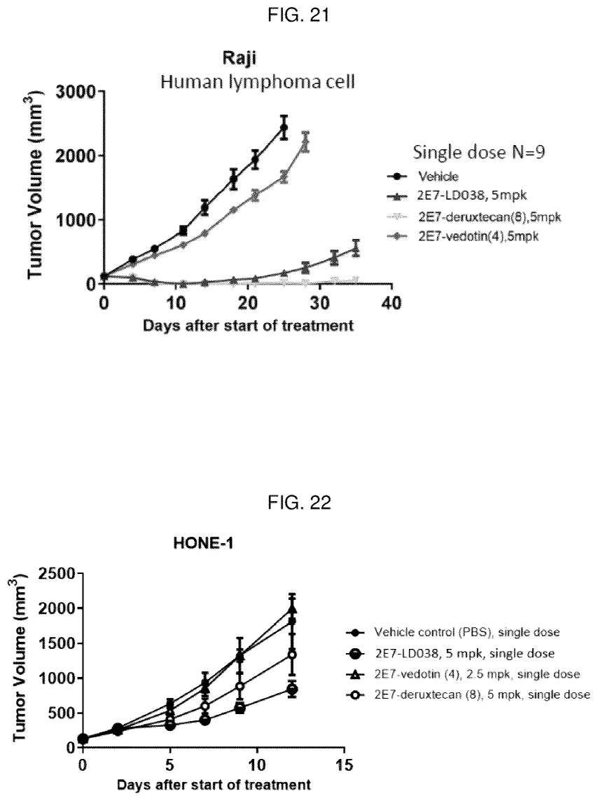

Antitumor activity of 2E7 conjugates with the benchmarking linker-drugs (see Table 7) was evaluated in CDX (cell-derived xenograft) models. Female BALB/c nude mice were inoculated subcutaneously at right flank with Caki-1 cells (ATCC, HTB-46, 3×10⁶ in 0.2 mL cell suspension) or Raji cells (from Betapharma, 5×10⁶ in 0.1 mL cell suspension) for tumor development. Five to eight days after tumor inoculation, mice with average tumor size 120-130 mm³ were selected and assigned into treatment groups for each model using stratified randomization based upon their tumor volumes (n=9-10 mice per group). The treatment initiated one day after randomization (randomization day defined as day 0 (DO)) and was in either single-dose (on day1) or multiple-dose (day1/day4/day8/day11) regimen via intravenous infusion of the 2E7 conjugates at 5 mg/kg. Tumor size and body weight were measured twice a week in two dimensions using a caliper, and the volume was expressed in mm³ using the formula: V=0.5 a×b² where a and b are the long and short diameters of the tumor, respectively. Tumor volume exceeding 2000 mm³ was defined as endpoint. Animal body weight was monitored as an indirect measure of toxicity. No mice showed significant weight loss in any of the study groups. There were no morbidity or death in the treatment duration. Compared to vehicle control group, both 2E7-LD038 and 2E7-deruxtecan (8-load) produced marked tumor growth inhibition with comparable potency in both Caki-1 and Raji models; 2E7-vedotin(4-load) exerted low-to-moderate antitumor activity and was much less potent compared to the two other ADCs in these models (FIGS. 18-21).

### Example 72: 2E7 Conjugates: In Vivo Efficacy in Cell Line-Derived Xenograft (CDX) Model for Nasopharyngeal Carcinoma (NPC)

Anti-tumor activity of 2E7 conjugates was evaluated in a CDX model for nasopharyngeal carcinoma (NPC) in mice using the HONE-1 nasopharyngeal carcinoma (NPC) cell line (BNCC Article number: BNCC338405). Female BALB/c nude mice were inoculated subcutaneously at right flank with HONE-1 cells (5×10⁶ in 0.1 mL cell suspension). 9 days after tumor inoculation, mice with average tumor size of 133 mm³ were selected and assigned into 4 treatment groups using stratified randomization (n=6 per group) based upon their tumor volume. A single dose of the test articles was administered one day after randomization (with randomization day defined as Day 0) via intravenous injection.

The tumor size and body weight were measured twice a week. Tumor volume (measured in two dimensions using a caliper) was expressed in mm³ using the formula: V=0.5 a×b² where a and b represent the long and short diameter of the tumor, respectively. Tumor volume exceeding 2000 mm³ was regarded as endpoint. Animal body weight was monitored as an indirect assessment of toxicity. None of the mice showed significant weight loss in any of the treatment groups.

There were no morbidity or death in the study duration. 2E7-LD038 produced marked tumor growth inhibition compared to the vehicle control; anti-tumor effect of 2E7-deruxtecan (DAR 8) was more modest compared to 2E7-LD038; 2E7-vedotin (DAR 4) did not elicit appreciable tumor growth inhibition compared to vehicle control (see, FIG. 22).

### Example 73: Anti-Tumor Activity of 2E7-LD038 Conjugate in Cell Line-Derived Xenograft (CDX) Models in Mice

Female BALB/c nude mice were inoculated subcutaneously at right flank with Caki-1 cells (ATCC, HTB-46, 3×10⁶ in 0.2 mL cell suspension) or female NOD-SCID mice were inoculated subcutaneously at right flank with Raji cells (ATCC, CCL-86, 5×10⁶ in 0.1 mL cell suspension) or 786-0 cells (BNCC, BNCC338472, 2×10⁶ cells in 0.1 mL medium) for tumor development. 5 to 18 days after tumor inoculation, mice with average tumor size of 110-130 mm³ were selected and assigned into 3 or 5 groups for each model using stratified randomization with 8-10 mice in each group based upon their tumor volumes. The treatments were started from one day after randomization (randomization day defined as day 0 (DO)) and were treated with a single (day1) or multiple (day1/day4/day8/day11) intravenous injection of 2E7-LD038 at dose(s) denoted in the graph.

The tumor sizes and body weight were measured twice a week in two dimensions using a caliper, and the volume was expressed in mm³ using the formula: V=0.5 a×b² where a and b are the long and short diameters of the tumor, respectively. Tumor volume exceeding 2000 mm³ was regarded as endpoint. Animal body weight was monitored as an indirect assessment of toxicity. No mice showed significant weight loss in any of the treatment groups.

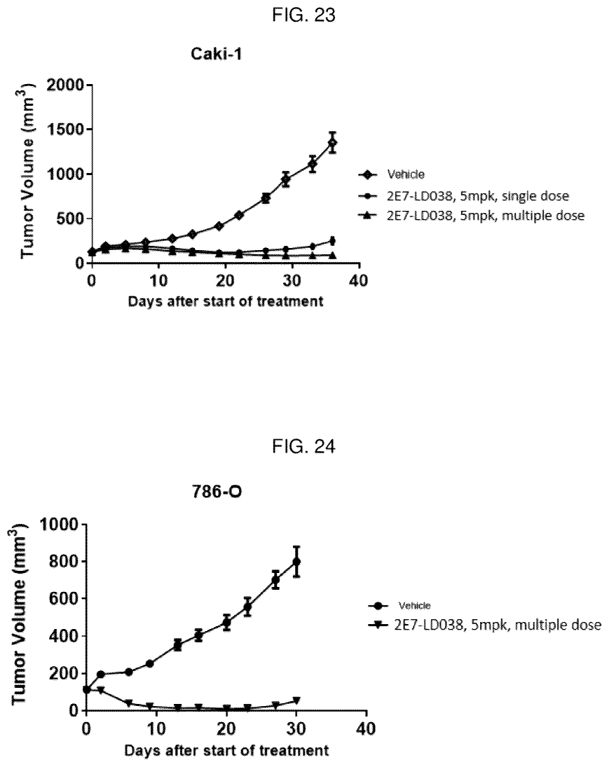

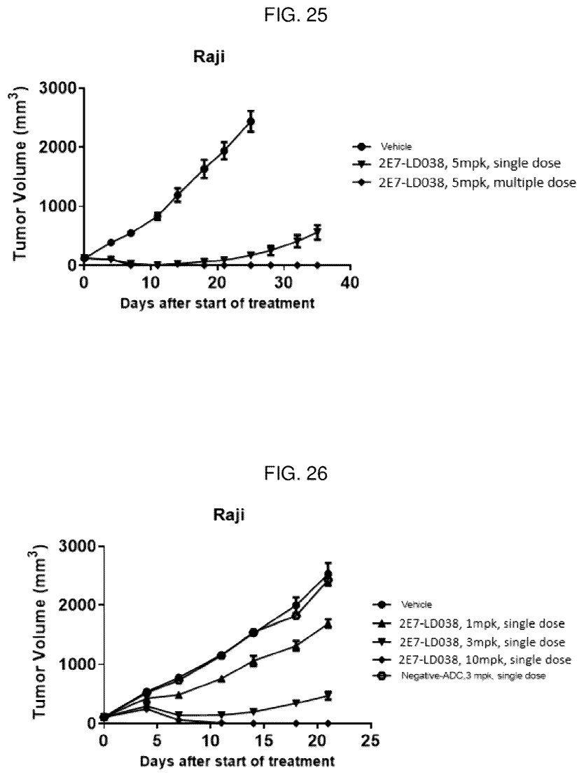

There were no morbidity and deaths in the study duration. 2E7-LD038 conjugate produced robust antitumor activity against Caki-1 (RCC), 786-O (RCC), Raji (lymphoma) xenografts (see, FIGS. 23-26). The non-binding control (Negative-ADC) in the Raji model had minimal activity in tumor growth-inhibition, demonstrating the CD70-mediated mode of action for non-binding control.

### Example 74: Anti-Tumor Activity of 2E7-LD038 Conjugate in PDX Models for Diffuse Large B Cell Lymphoma (DLBCL)

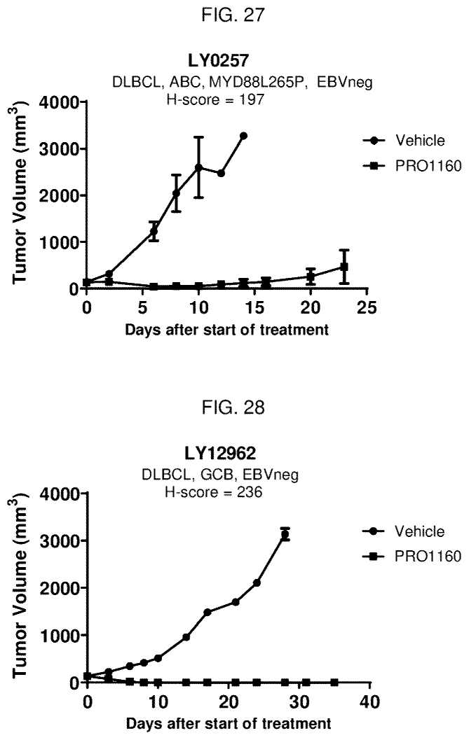

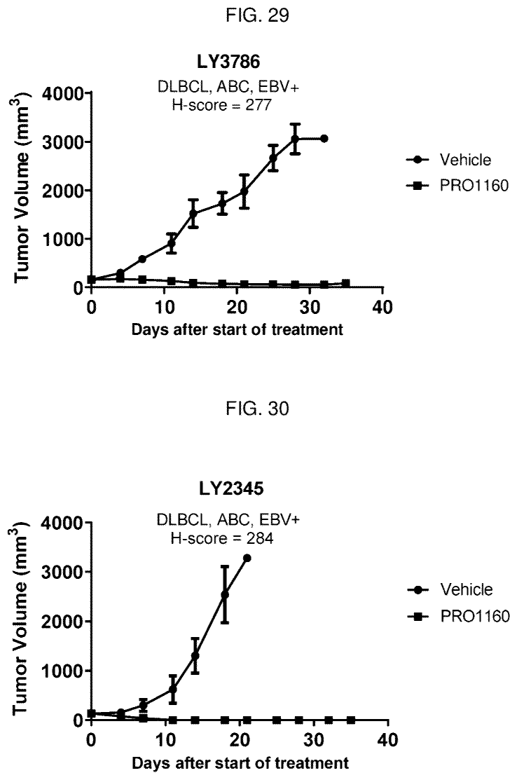

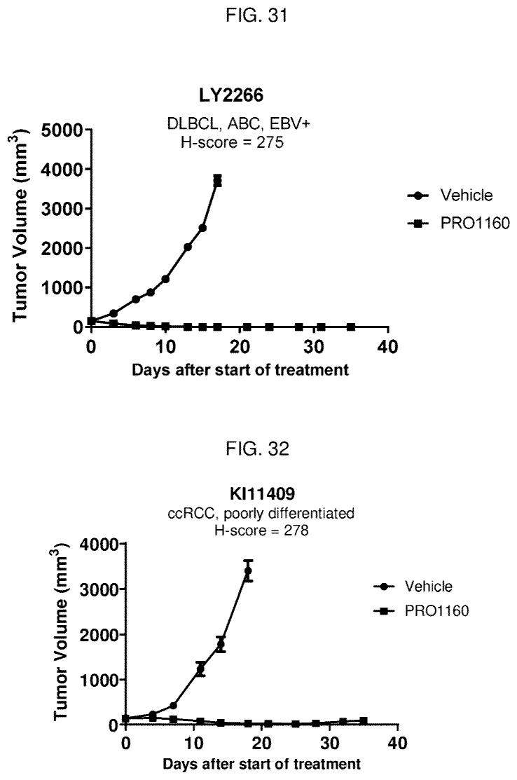

The anti-tumor activity of 2E7-LD038 conjugate (DAR8) was evaluated in patient-derived xenograft (PDX) models for diffuse large B cell lymphoma (DLBCL). NOD/SCID mice were subcutaneously implanted with one of five different patient-derived xenografts representing DLBCL. The DLBCL sample for studying LY0257 was of the ABC type, Epstein Bar virus (EBV) negative and had an H-score of 197 (FIG. 27). The sample also contained the MYD88 L265P mutation. The DLBCL sample for studying LY12962 was of the GCB type, EBV negative and had an H-score of 236 (FIG. 28). The DLBCL sample for studying LY3786 was of the ABC type, EBV positive and had an H-score of 277 (FIG. 29). The DLBCL sample for studying LY2345 was of the ABC type, EBV positive and had an H-score of 284 (FIG. 30). The DLBCL sample for studying LY2266 was of the ABC type, EBV positive and had an H-score of 275 (FIG. 31). The implanted mice were treated with a single dose (5 mg/kg) of 2E7-LD038 (n=3) or vehicle (PBS) control (n=2) on day 0 (day of implant) via intravenous injection.

Tumor volume (measured in two dimensions using a caliper) was expressed in mm³ and calculated according to the following equation: tumor volume (mm³)=(length×width²)/2.

The CD70 H-score was determined via standard methodology with anti-CD70 rabbit monoclonal ([EPR26536-122](ab300083)) used as the immunohistochemistry (IHC) staining antibody.

2E7-LD038 conjugate was well tolerated at 5 mg/kg in mice. Antitumor activity of 2E7-LD038 conjugate was observed in all five PDX models of DLBCL (FIGS. 27-31).

### Example 75: Anti-Tumor Activity of 2E7-LD038 Conjugate in PDX Models for Clear Cell Renal Cell Carcinoma (ccRCC)

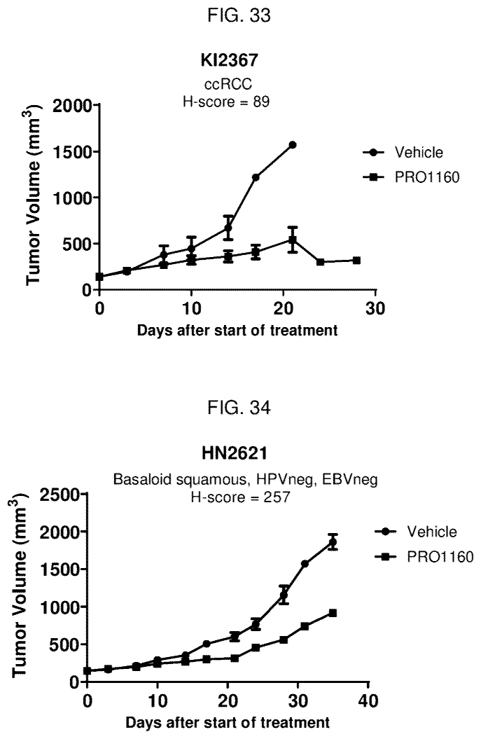

The anti-tumor activity of 2E7-LD038 conjugate (DAR8) was evaluated in PDX models for clear cell renal cell carcinoma (ccRCC). NOD/SCID mice were subcutaneously implanted with one of two patient-derived xenografts representing ccRCC. The ccRCC sample for studying KI11409 was poorly differentiated and had an H-score of 278 (FIG. 32). The ccRCC sample for studying KI2367 had an H-score of 89 (FIG. 33). The implanted mice were treated with a single dose (5 mg/kg) of 2E7-LD038 (n=3) or vehicle (PBS) control (n=2) on day 0 (day of implant) via intravenous injection.

Tumor volume (measured in two dimensions using a caliper) was expressed in mm³ and calculated according to the following equation: tumor volume (mm³)=(length×width²)/2.

The CD70 H-score was derived via a standard methodology with anti-CD70 rabbit monoclonal ([EPR26536-122](ab300083)) used as the immunohistochemistry (IHC) staining antibody.

2E7-LD038 conjugate was well tolerated at 5 mg/kg in mice. Antitumor activity of 2E7-LD038 was observed in both PDX models of ccRCC (FIGS. 32-33).

### Example 76: Anti-Tumor Activity of 2E7-LD038 Conjugate in a PDX Model for Head and Neck Squamous Cell Carcinoma (HNSCC)

Anti-tumor activity of 2E7-LD038 conjugate (DAR8) was evaluated in a PDX model for head and neck squamous cell carcinoma (HNSCC). NOD/SCID mice were subcutaneously implanted with patient-derived xenografts representing HNSCC. The sample was a basaloid squamous carcinoma, human papillomavirus (HPV) negative, EBV negative and had an H-score of 257. The implanted mice were treated with a single dose (5 mg/kg) of 2E7-LD038 conjugate (n=3) or vehicle (PBS) control (n=2) on day 0 (day of implant) via intravenous injection.

Tumor volume (measured in two dimensions using a caliper) was expressed in mm³ and calculated according to the following equation: tumor volume (mm³)=(length×width²)/2.

The CD70 H-score was derived via a standard methodology with anti-CD70 rabbit monoclonal ([EPR26536-122](ab300083)) used as the immunohistochemistry (IHC) staining antibody.

2E7-LD038 conjugate was well tolerated at 5 mg/kg in mice. Antitumor activity of 2E7-LD038 was observed in the PDX model of HNSCC (FIG. 34).

SEQUENCE LISTING
69A7 VH amino acid sequence
SEQ ID NO: 1
QVQLQESGPG LVKPSETLSL TCTVSGGSVS SDYYYWSWIR QPPGKGLEWL GYIYYSGSTN
YNPSLKSRVT ISVDTSKNQF SLKLRSVTTA DTAVYYCARG DGDYGGNCFD YWGQGTLVTV
SS
69A7 VL amino acid sequence
SEQ ID NO: 2
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RSNWPLTFGG GTKVEIK
2A4 VH amino acid sequence
SEQ ID NO: 3
QVQLQESGPG LVKPSETLSL TCTVSGGSVS SDYYYWSWIR QPPGKGLEWL GYIYYSGSTN
YNPSLKSRVT ISVDTSKNQF SLKLRSVTTA DTAVYYCARG DGDYGGNVFP YWGQGTLVTV
SS
2A4 VL amino acid sequence
SEQ ID NO: 4
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RSNWPLTFGG GTKVEIK
1H8 VH amino acid sequence
SEQ ID NO: 5
QVQLQESGPG LVKPSETLSL TCTVSGGSVS SDYYYWSWIR QPPGKGLEWL GYIYYSGSTN
YNPSLKSRVT ISVDTSKNQF SLKLRSVTTA DTAVYYCARG DGDEMGVCFD YWGQGTLVTV
SS
1H8 VL amino acid sequence
SEQ ID NO: 6
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RSNWPLTFGG GTKVEIK
2E7 VH amino acid sequence
SEQ ID NO: 7
QVQLQESGPG LVKPSETLSL TCTVSGGSVS SDYYYWSWIR QPPGKGLEWL GYIYYSGSTN
YNPSLKSRVT ISVDTSKNQF SLKLRSVTTA DTAVYYCARG DGDFLGVCFD YWGQGTLVTV SS
2E7 VL amino acid sequence
SEQ ID NO: 8
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RSNWPLTFGG GTKVEIK
2D2 VH amino acid sequence
SEQ ID NO: 9
QVQLQESGPG LVKPSETLSL TCTVSGGSVS SDYYYWSWIR QPPGKGLEWL GYIYYSGSTN
YNPSLKSRVT ISVDTSKNQF SLKLRSVTTA DTAVYYCARG DGDYGGNCFD YWGQGTLVTV
SS
2D2 VL amino acid sequence
SEQ ID NO: 10
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RLKFPLTFGG GTKVEIK
1A4 VH amino acid sequence
SEQ ID NO: 11
QVQLQESGPG LVKPSETLSL TCTVSGGSVY SGYYYWSWIR QPPGKGLEWL GYFSLSGSTN
YNPSLKSRVT ISVDTSKNQF SLKLRSVTTA DTAVYYCARG DGDYGGNCFD YWGQGTLVTV
SS
1A4 VL amino acid sequence
SEQ ID NO: 12
EIVLTQSPAT LSLSPGERAT LSCRASQSVS SYLAWYQQKP GQAPRLLIFD ASNRATGIPA
RFSGSGSGTD FTLTISSLEP EDFAVYYCQQ RSNWPLTFGG GTKVEIK
2A4 HCDR3 amino acid sequence
SEQ ID NO: 13
GDGDYGGNVF PY
1H8 HCDR3 amino acid sequence
SEQ ID NO: 14
GDGDFMGVCF DY
2E7 HCDR3 amino acid sequence
SEQ ID NO: 15
GDGDFLGVCF DY
1A4 HCDR1 amino acid sequence
SEQ ID NO: 16
YSGYYYWS
1A4 HCDR2 amino acid sequence
SEQ ID NO: 17
YFSLSGSTNY NPSLKS
2D2 LCDR3
SEQ ID NO: 18
QQRLKFPLT
h1F6 VH amino acid sequence
SEQ ID NO: 19
QVQLVQSGAE VKKPGASVKV SCKASGYTFT NYGMNWVRQA PGQGLKWMGW
INTYTGEPTY ADAFKGRVTM TRDTSISTAY MELSRLRSDD TAVYYCARDY GDYGMDYWGQ
GTTVTVSS
h1F6 VL amino acid sequence
SEQ ID NO: 20
DIVMTQSPDS LAVSLGERAT INCRASKSVS TSGYSFMHWY QQKPGQPPKL LIYLASNLES
GVPDRFSGSG SGTDFTLTIS SLQAEDVAVY YCQHSREVPW TFGQGTKVEI K
HCDR1 amino acid sequence
SEQ ID NO: 21
SSDYYYWS
HCDR2 amino acid sequence
SEQ ID NO: 22
YIYYSGSTNY NPSLKS
HCDR3 amino acid sequence
SEQ ID NO: 23
GDGDYGGNCF DY
LCDR1 amino acid sequence
SEQ ID NO: 24
RASQSVSSYLA
LCDR2 amino acid sequence
SEQ ID NO: 25
DASNRAT
LCDR3 amino acid sequence
SEQ ID NO: 26
QQRSNWPLT
SEQ ID NO: 27
(GGGGS)
human IgG1 heavy chain UniProt P01857-1
SEQ ID NO: 28
ASTKGPSVFP LAPSSKSTSG GTAALGCLVK DYFPEPVTVS WNSGALTSGV
HTFPAVLQSS GLYSLSSVVT VPSSSLGTQT YICNVNHKPS NTKVDKKVEP
KSCDKTHTCP PCPAPELLGG PSVFLFPPKP KDTLMISRTP EVTCVVVDVS
HEDPEVKFNW YVDGVEVHNA KTKPREEQYN STYRVVSVLT VLHQDWLNGK
EYKCKVSNKA LPAPIEKTIS KAKGQPREPQ VYTLPPSRDE LTKNQVSLTC
LVKGFYPSDI AVEWESNGQP ENNYKTTPPV LDSDGSFFLY SKLTVDKSRW
QQGNVFSCSV MHEALHNHYT QKSLSLSPGK
human Kappa light chain UniProt P01834-1
SEQ ID NO: 29
RTVAAPSVFI FPPSDEQLKS GTASVVCLLN NFYPREAKVQ WKVDNALQSG
NSQESVTEQD SKDSTYSLSS TLTLSKADYE KHKVYACEVT HQGLSSPVTK
SFNRGEC
hexa-histidine
SEQ ID NO: 30
HHHHHH
SEQ ID NO: 31
GCCGCCACC
SEQ ID NO: 32
MGWSCIILFLVATATGVHS
SEQ ID NO: 33
LPXTG
SEQ ID NO: 34
(Succinimid—3—yl—N)—(CH2)n—C(═O)—GGFG—NH—CH2—O—CH2—(C═O)—
SEQ ID NO: 35
GGFG
SEQ ID NO: 36
ALAL

1. A conjugate comprising: a Binding unit bound to one or more Drug units by one or more Linkers, wherein the Binding unit includes at least a portion of an anti-CD70 antibody.

2. (canceled)

3. A conjugate comprising: a Binding unit bound to one or more Drug units by one or more Linkers, wherein:
(1) the Binding unit comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of:
a. SEQ ID NO:21, SEQ ID NO:22, SEQ ID NO:13, SEQ ID NO:24, SEQ ID NO:25 and SEQ ID NO:26, respectively;
b. SEQ ID NO:21, SEQ ID NO:22, SEQ ID NO:14, SEQ ID NO:24, SEQ ID NO:25 and SEQ ID NO:26, respectively;
c. SEQ ID NO:21, SEQ ID NO:22, SEQ ID NO:15, SEQ ID NO:24, SEQ ID NO:25 and SEQ ID NO:26, respectively;
d. SEQ ID NO:21, SEQ ID NO:22, SEQ ID NO:23, SEQ ID NO:24, SEQ ID NO:25 and SEQ ID NO:18, respectively; and
e. SEQ ID NO:16, SEQ ID NO:17, SEQ ID NO:23, SEQ ID NO:24, SEQ ID NO:25 and SEQ ID NO:26; respectively;
(2) each Linker has the following formula (I): 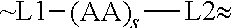
or a salt thereof, wherein:
L1 is a Stretcher unit covalently bound to the Binding unit, wherein the wavy (˜) line indicates an attachment site for the Binding unit;
AA is an Amino Acid unit having from 1 to 12 subunits;
s is 0 or 1;
L2 is a Linker Subunit having from 1 to 4 attachment sites for a Drug unit, wherein the double wavy (≈) line indicates an attachment site for the Drug Unit; and
wherein at least one Polar unit is present within the Amino Acid unit, the Linker Subunit, the Stretcher unit, or combinations thereof, and wherein the Polar unit(s) is selected from Sugar units, PEG units, Carboxyl units, and combinations thereof; and
(3) each Drug unit is covalently attached to each Linker Subunit at (≈).

4. The conjugate of claim 3, wherein the VH and VL regions of the Binding unit have amino acid sequences that are selected from the pairs of amino acid sequences set forth in the group consisting of:
SEQ ID NO:3 and SEQ ID NO:4;
SEQ ID NO:5 and SEQ ID NO:6;
SEQ ID NO:7 and SEQ ID NO:8;
SEQ ID NO:9 and SEQ ID NO:10; and
SEQ ID NO:11 and SEQ ID NO:12; respectively.

5. The conjugate of claim 3, wherein the VH and VL regions of the Binding unit have amino acid sequences that are selected from the pairs of amino acid sequences set forth in the group consisting of:
SEQ ID NO:3 and SEQ ID NO:4;
SEQ ID NO:5 and SEQ ID NO:6;
SEQ ID NO:7 and SEQ ID NO:8;
SEQ ID NO:9 and SEQ ID NO:10; and
SEQ ID NO:11 and SEQ ID NO:12; respectively,
wherein the heavy and light chain framework regions are optionally modified with from 1 to 8 amino acid substitutions, deletions or insertions in the framework regions.

6. The conjugate of claim 3, wherein HCDR1, HCDR2 and HCDR3 and LCDR1, LCDR2 and LCDR3 of the Binding unit have the amino acid sequences set forth in SEQ ID NO:21, SEQ ID NO:22, and SEQ ID NO:15, and SEQ ID NO:24, SEQ ID NO:25 and SEQ ID NO:26, respectively.

7. The conjugate of claim 3, wherein the framework regions of the Binding unit are human framework regions.

8-9. (canceled)

10. The conjugate of claim 3, wherein the Binding unit further comprises a heavy chain constant region.

11. The conjugate of claim 10, wherein heavy chain constant region of the Binding unit is of the IgG isotype.

12. The conjugate of claim 11, wherein the heavy chain constant region of the Binding unit is an IgG1 constant region.

13. (canceled)

14. The conjugate of claim 12, wherein the IgG1 constant region of the Binding unit has the amino acid sequence set forth in SEQ ID NO:28.

15. The conjugate of claim 3, wherein the Binding unit further comprises a light chain constant region.

16. The conjugate of claim 15, wherein the light chain constant region of the Binding unit is of the kappa isotype.

17. The conjugate of claim 16, wherein the light chain constant region of the Binding unit has the amino acid sequence set forth in SEQ ID NO:29.

18. The conjugate of claim 10, wherein the heavy chain constant region of the Binding unit further comprises at least one amino acid modification that decreases binding affinity to human Fc receptor, such as FcgammaRIII.

19-46. (canceled)

47. The conjugate of claim 3, comprising a PEG unit having a formula selected from: 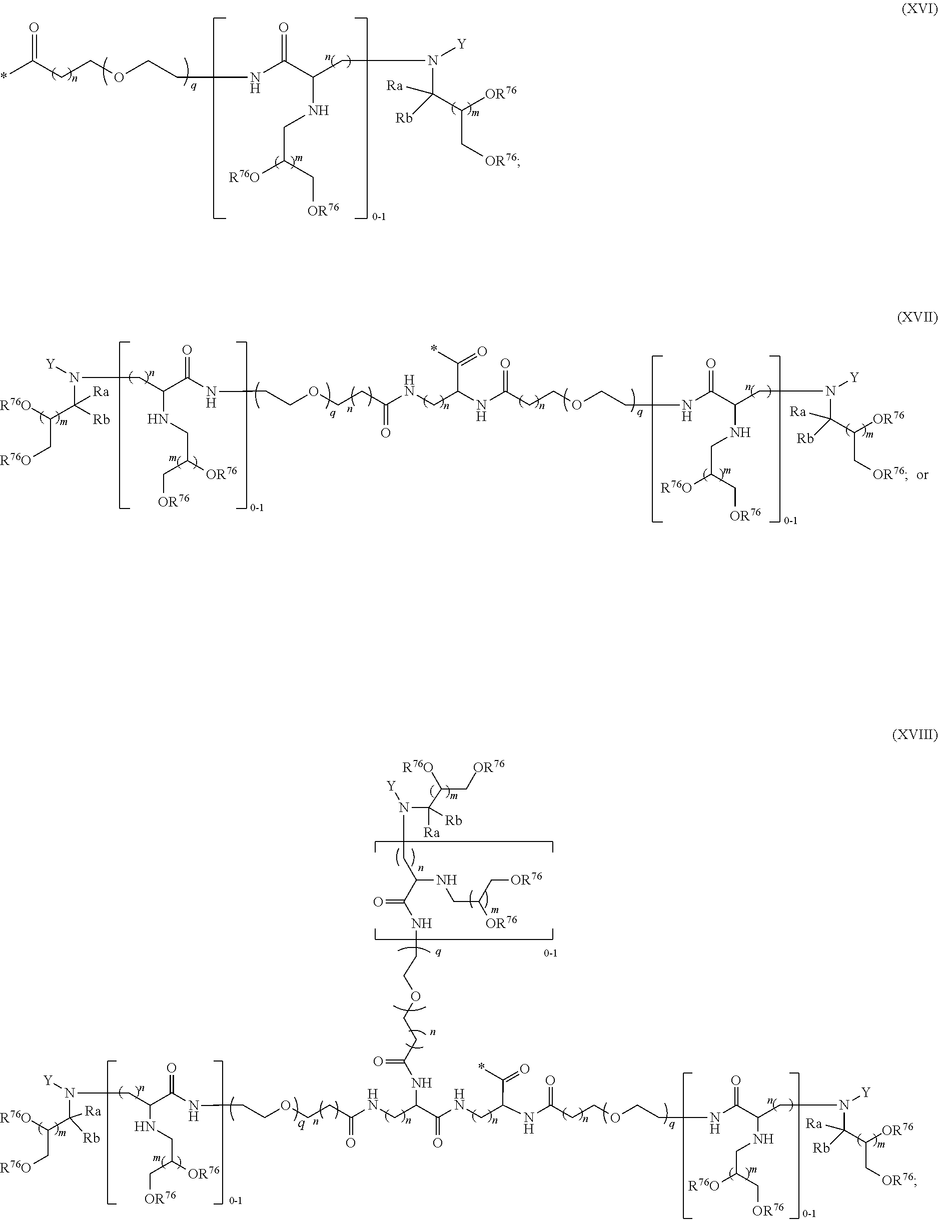
or a stereoisomer or salt thereof, wherein:
each Y is independently R⁷⁶ or 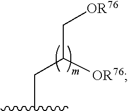
each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

48. The conjugate of claim 47, wherein the PEG unit has a formula selected from: 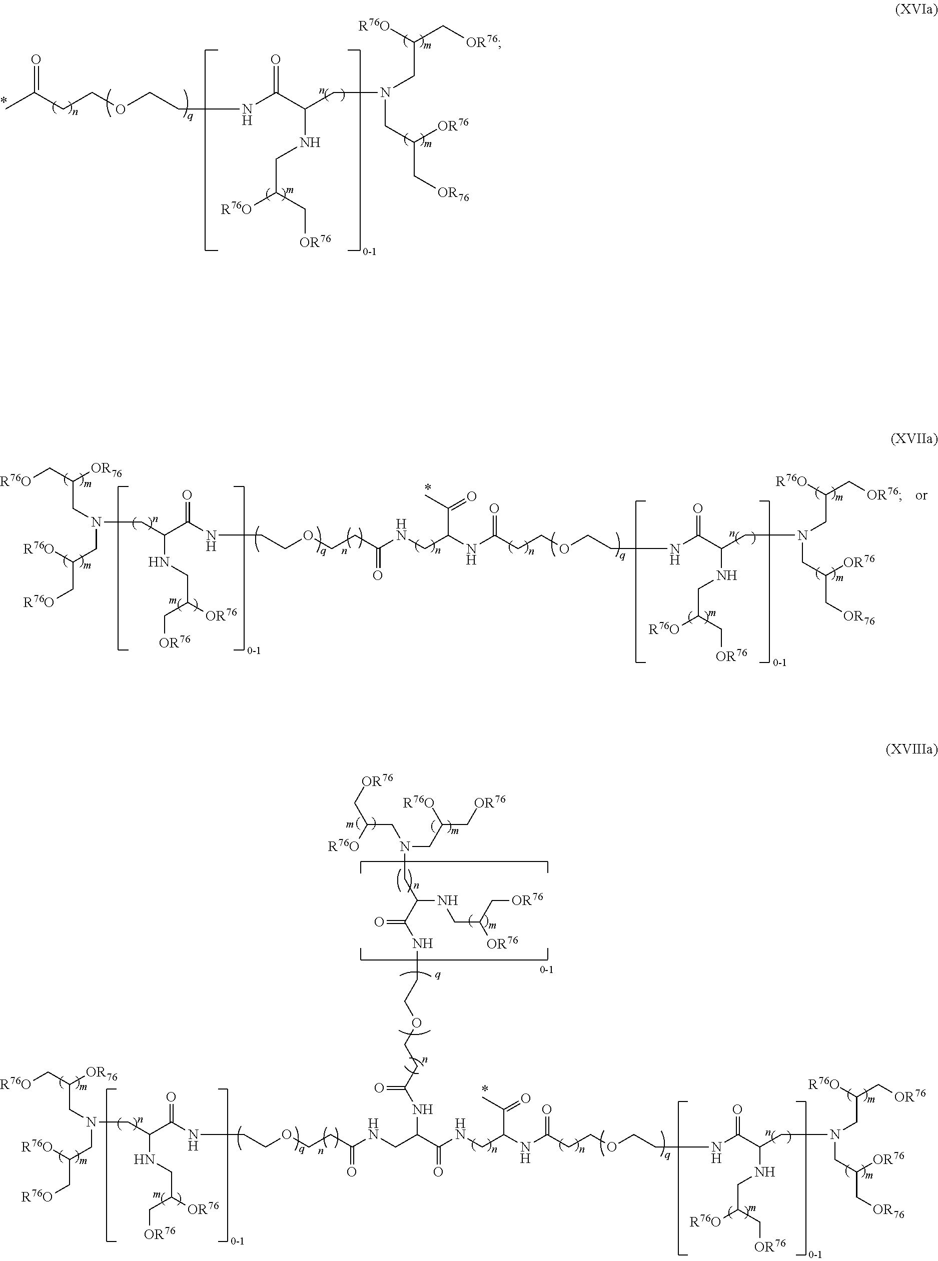
or a stereoisomer or salt thereof, wherein:
each R⁷⁶ is independently H, acetyl, —P(═O)(OH)₂, or —(CH₂)ᵥS(═O)₂(OH);
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

49-70. (canceled)

71. The conjugate of claim 3, wherein Linker Subunit L2 is a cleavable linker unit.

72. The conjugate of claim 71, wherein Linker Subunit L2 comprises a peptide that is cleavable by an intracellular protease.

73. The conjugate of claim 72, wherein the cleavable peptide comprises a valine-citrulline peptide, a valine-alanine peptide, a valine-lysine peptide, a phenylalanine-lysine peptide, or a glycine-glycine-phenylalanine-glycine peptide.

74-79. (canceled)

80. The conjugate of claim 79, wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG)) peptide or a Lys(PEG)-glycine-glycine-phenylalanine-glycine peptide, wherein Lys(PEG) and Cit(PEG) comprise a PEG unit attached to a lysine residue or a citrulline residue, respectively.

81. The conjugate of claim 71, wherein the cleavable peptide is attached to a para-aminobenzyl alcohol self immolative group (PABA).

82-87. (canceled)

88. The conjugate of claim 3, wherein each Drug unit is selected from a cytotoxic agent, an immune modulatory agent, a nucleic acid, a growth inhibitory agent, a PROTAC, a toxin, a radioactive isotope, and a chelating ligand.

89. (canceled)

90. The conjugate of claim 3, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

91. The conjugate of claim 3, wherein the drug is a cytotoxic agent.

92-95. (canceled)

96. The conjugate of claim 95, wherein the cytotoxic agent is exatecan.

97-117. (canceled)

118. The conjugate of claim 3, selected from the following: 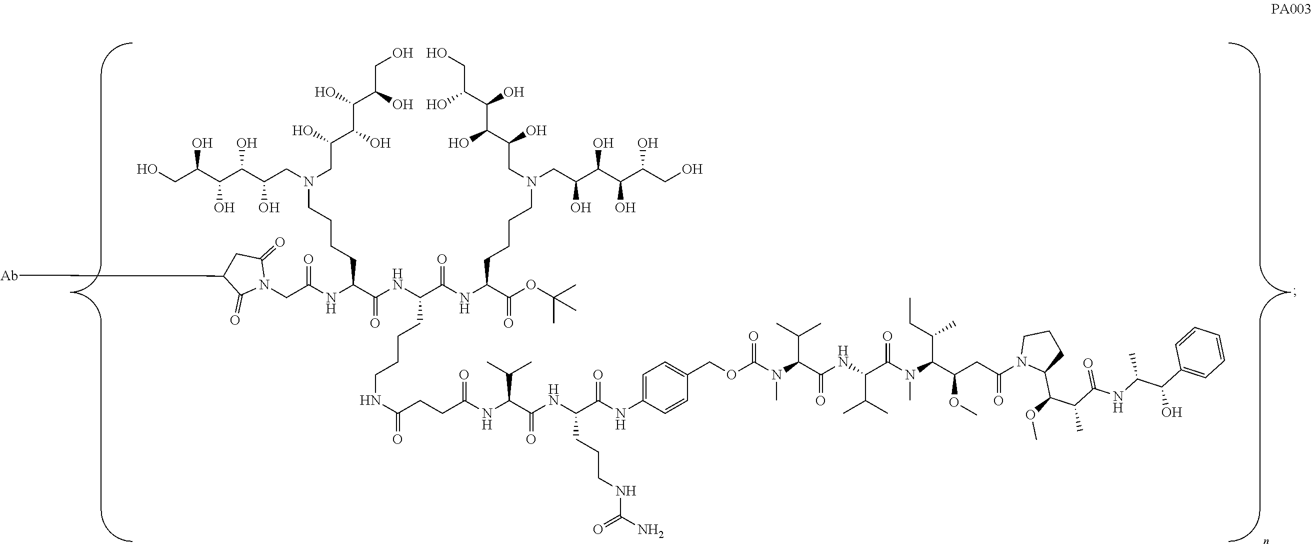 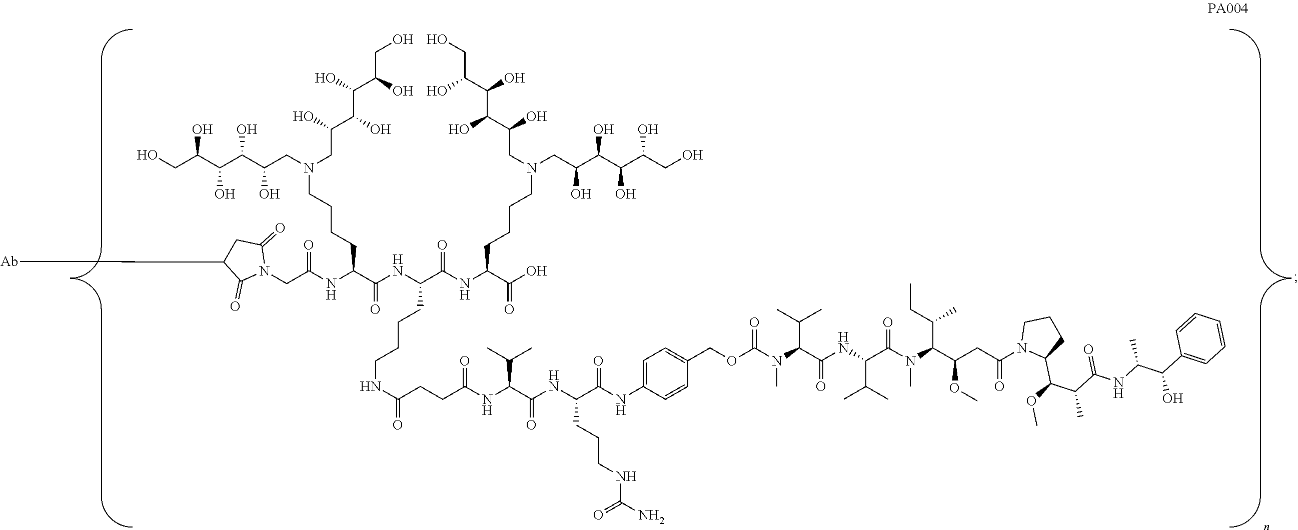 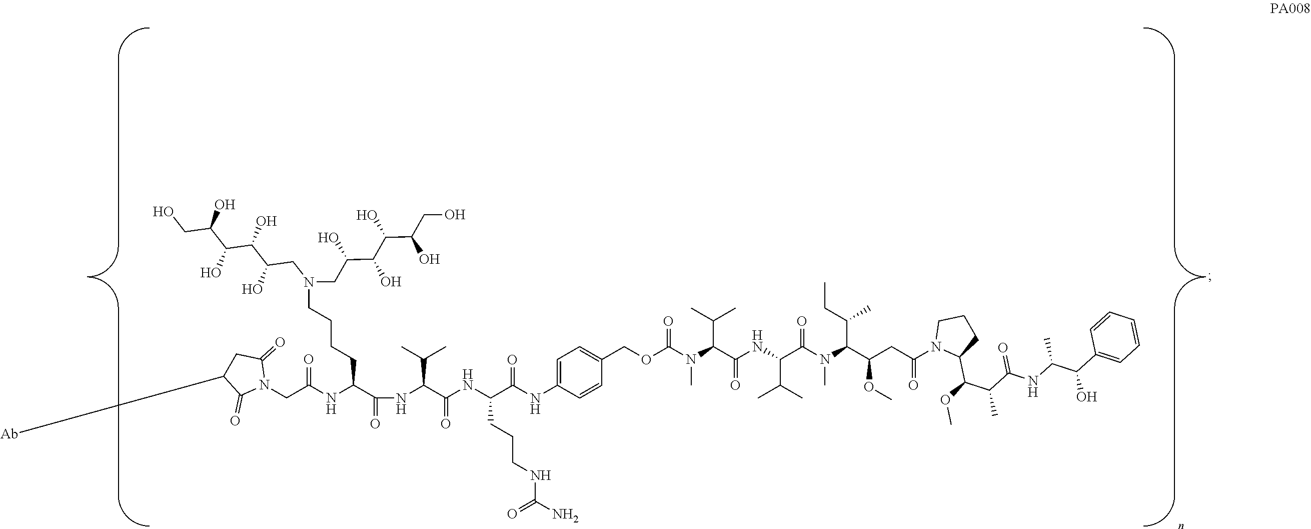 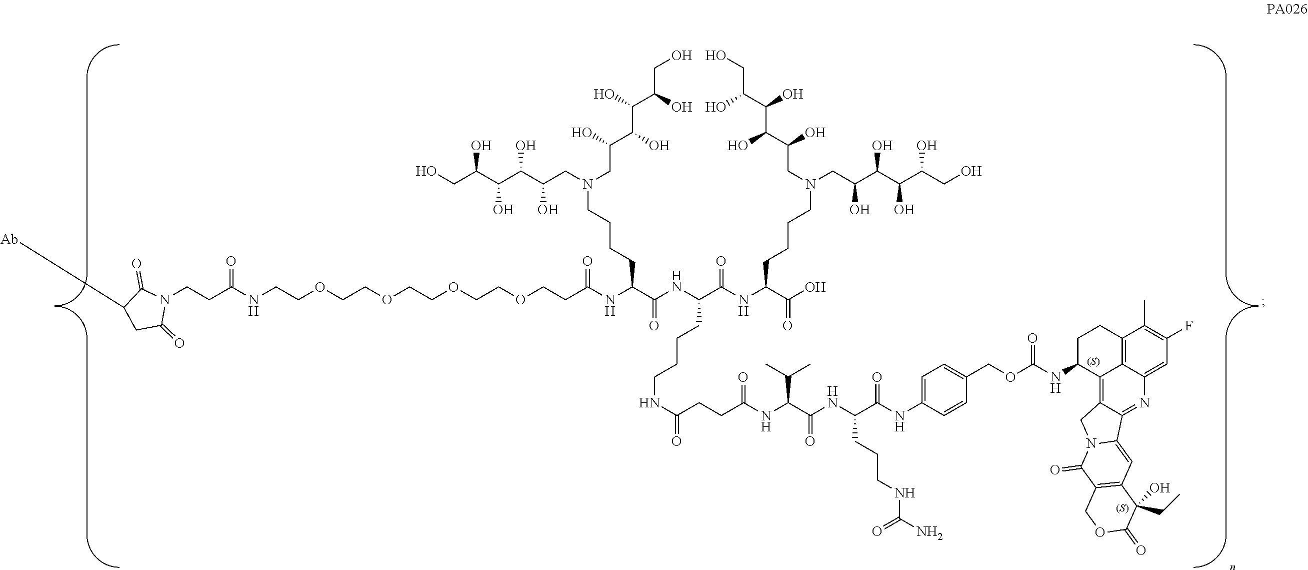 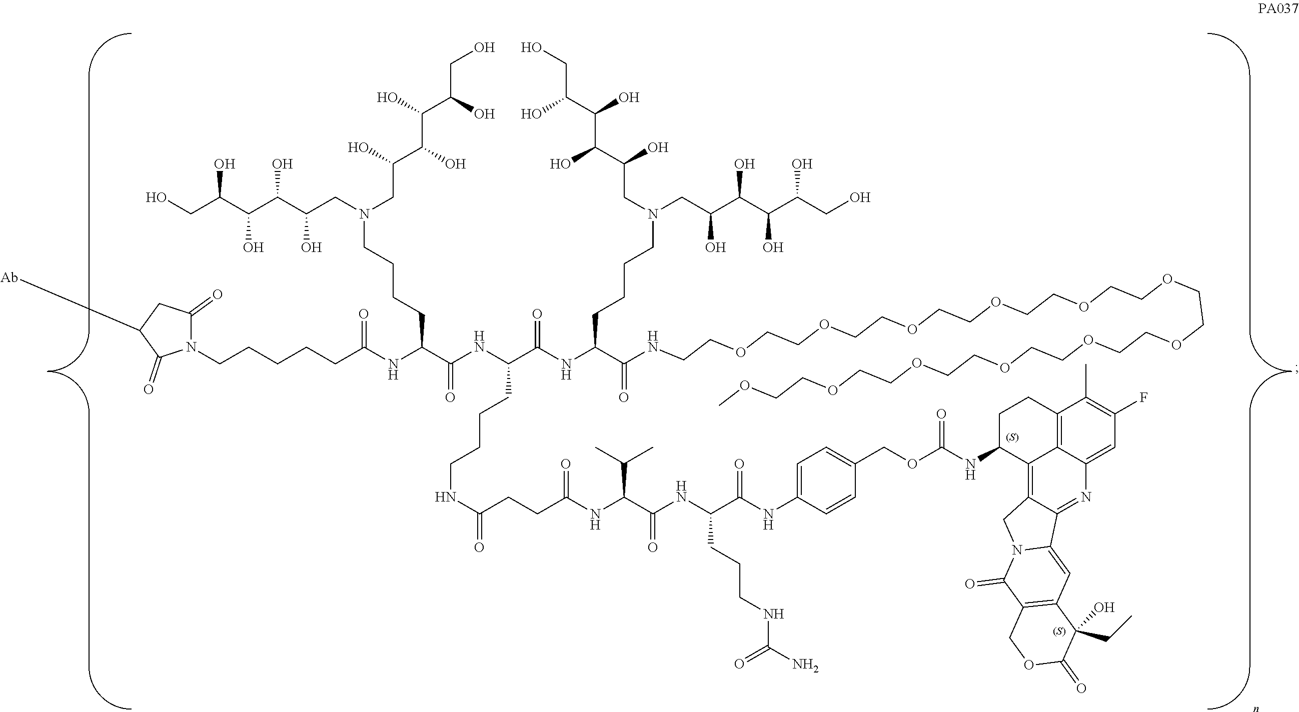 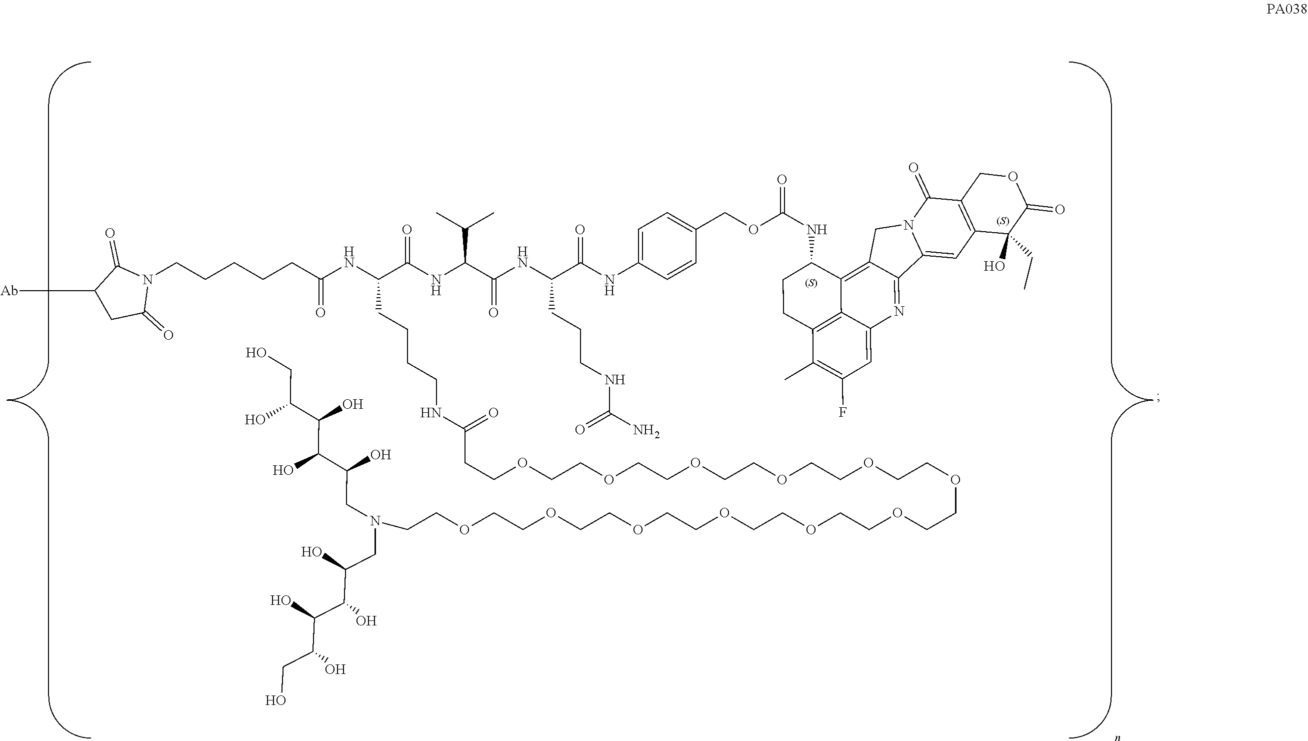 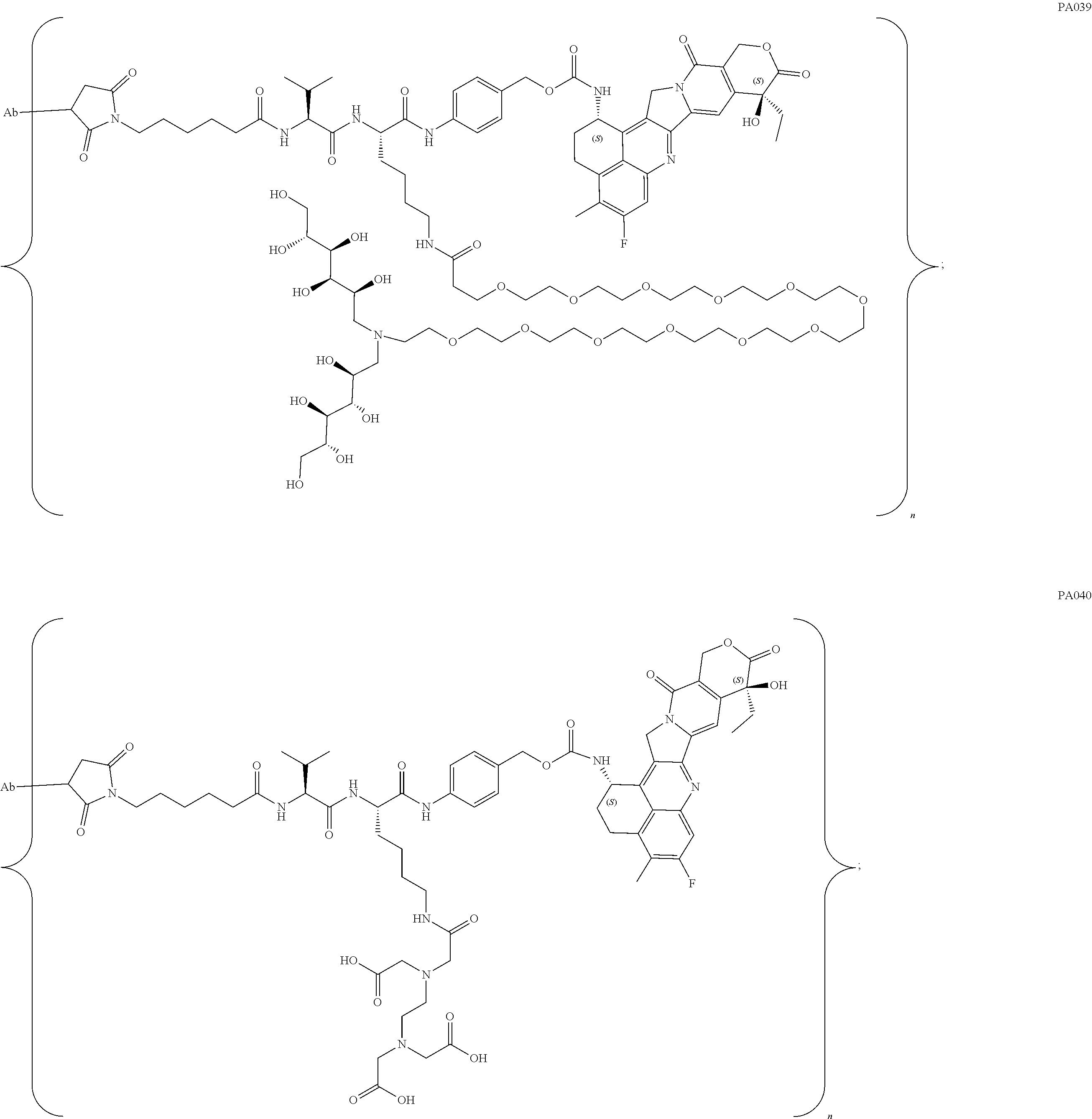 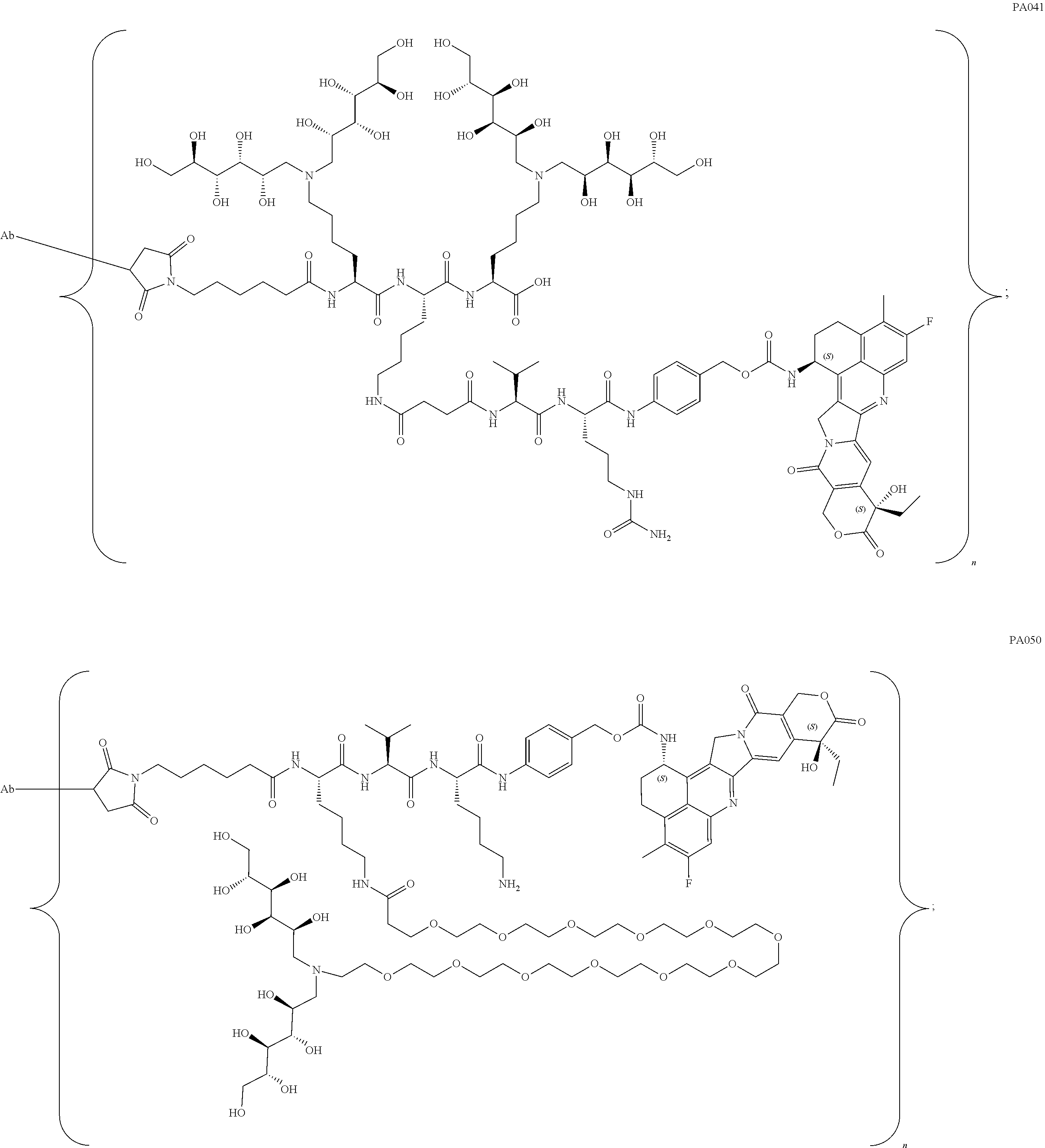 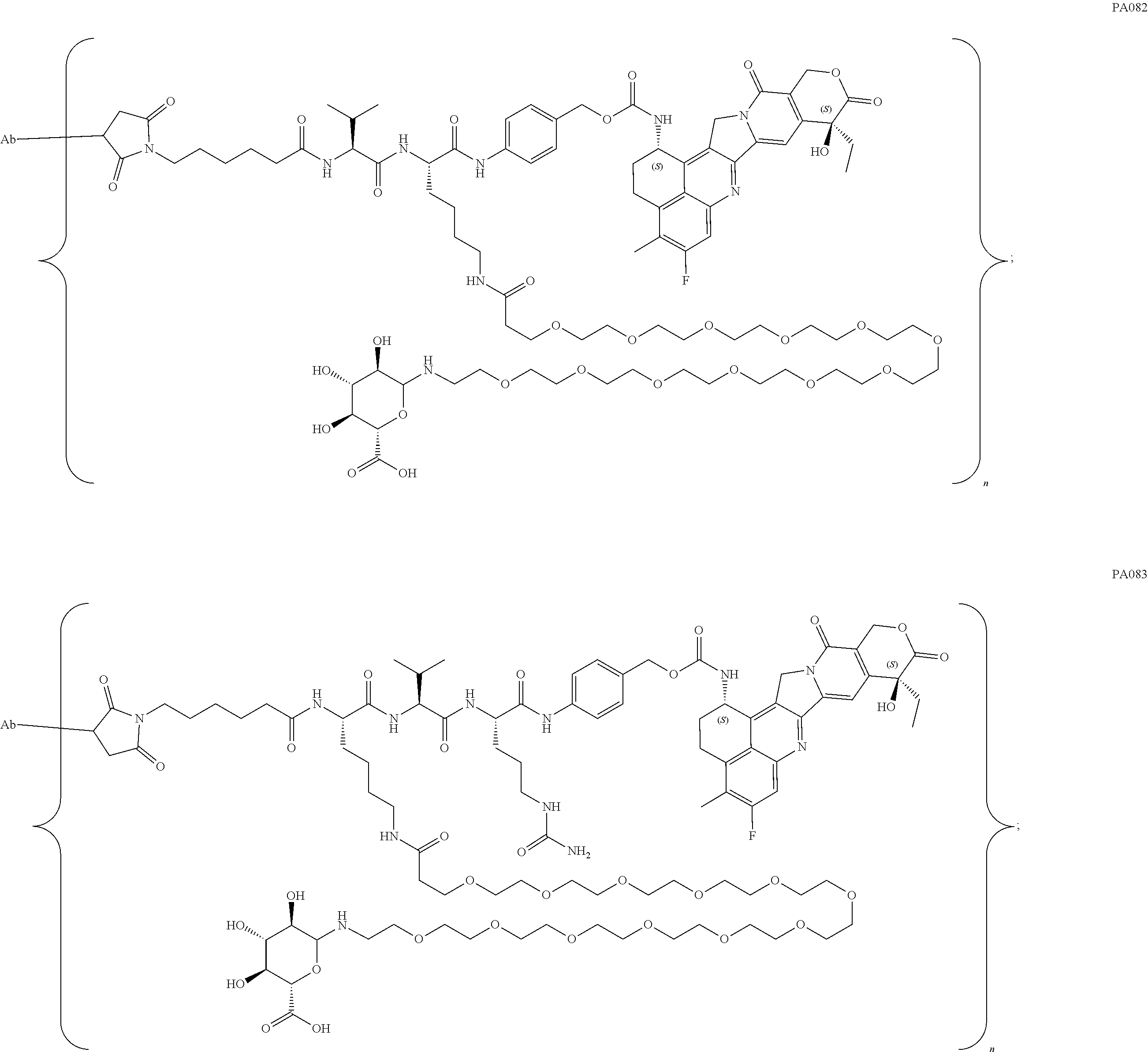 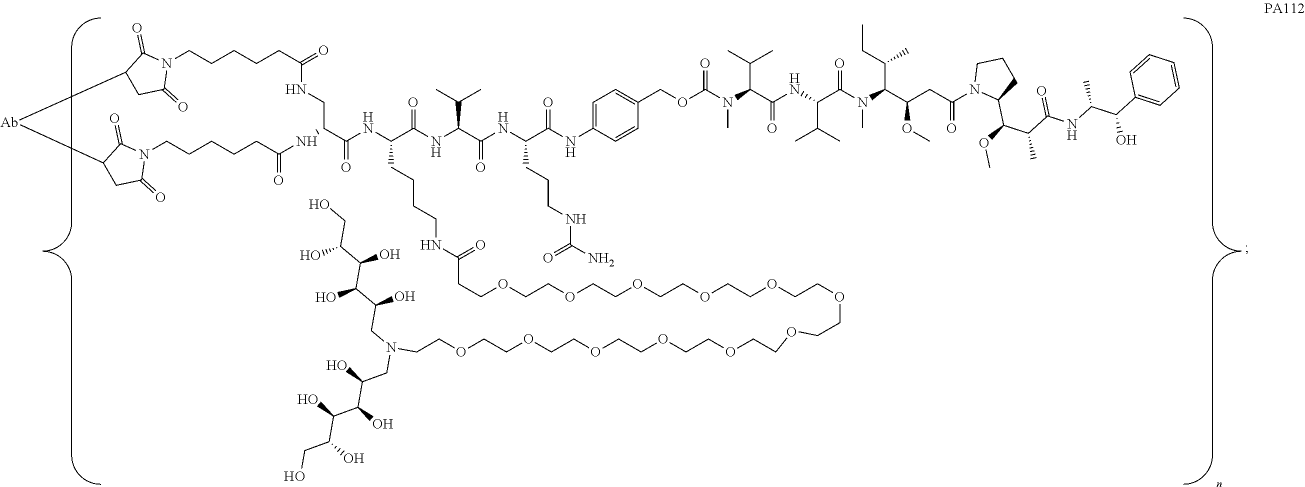 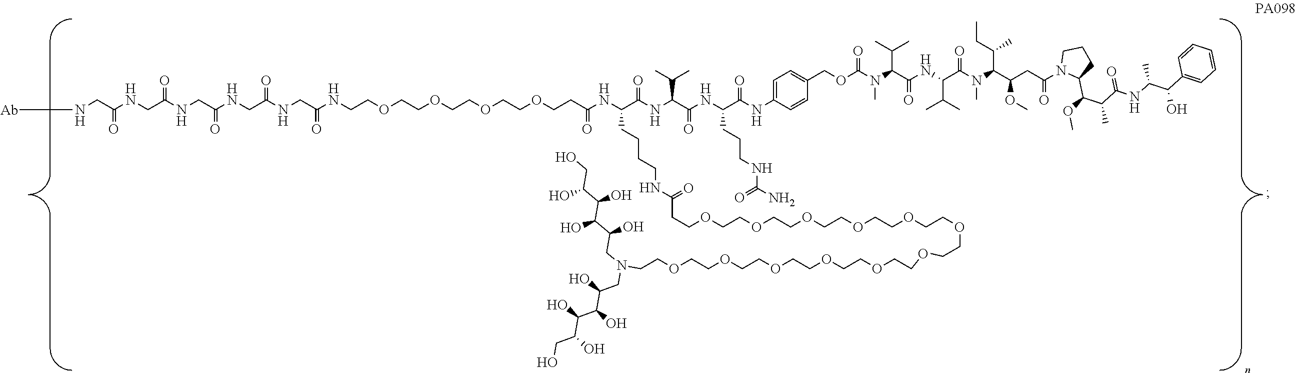 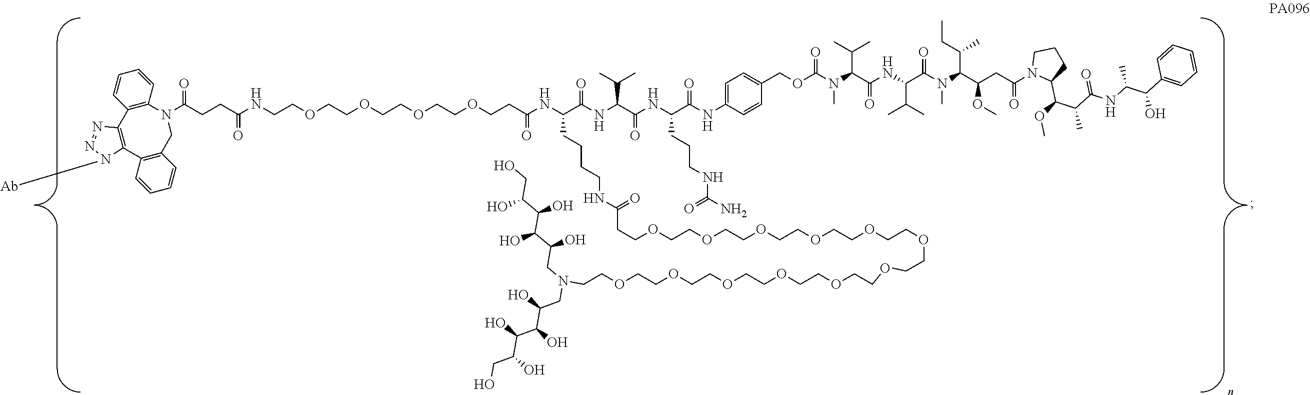 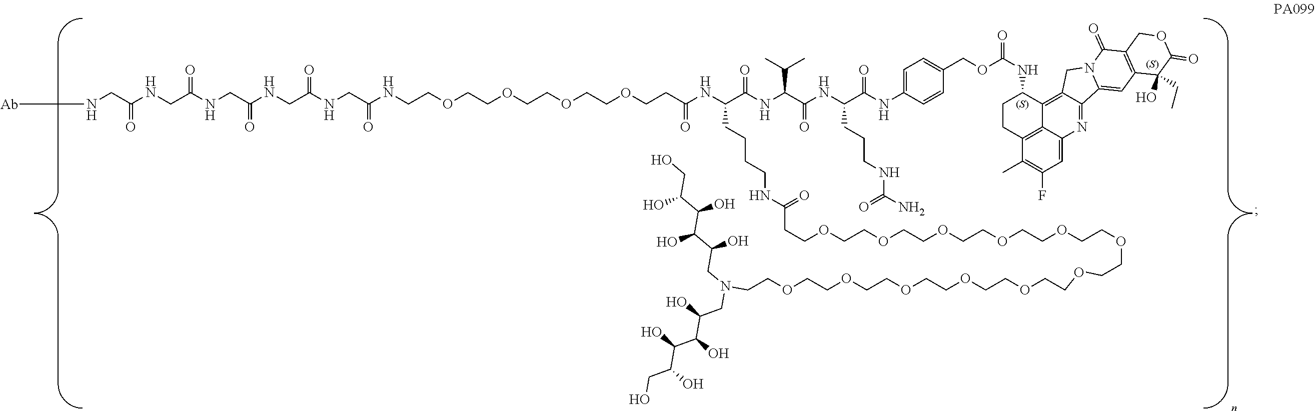 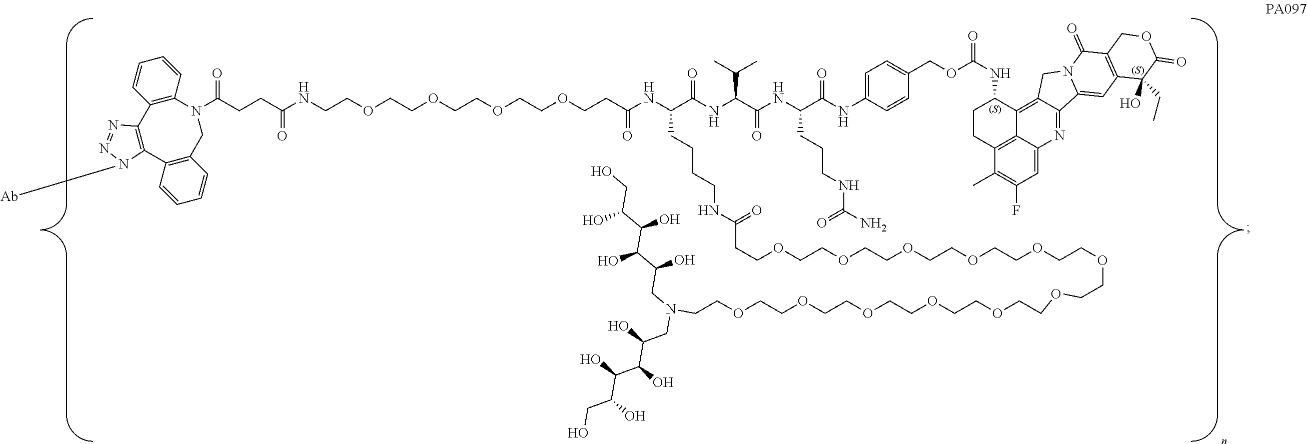 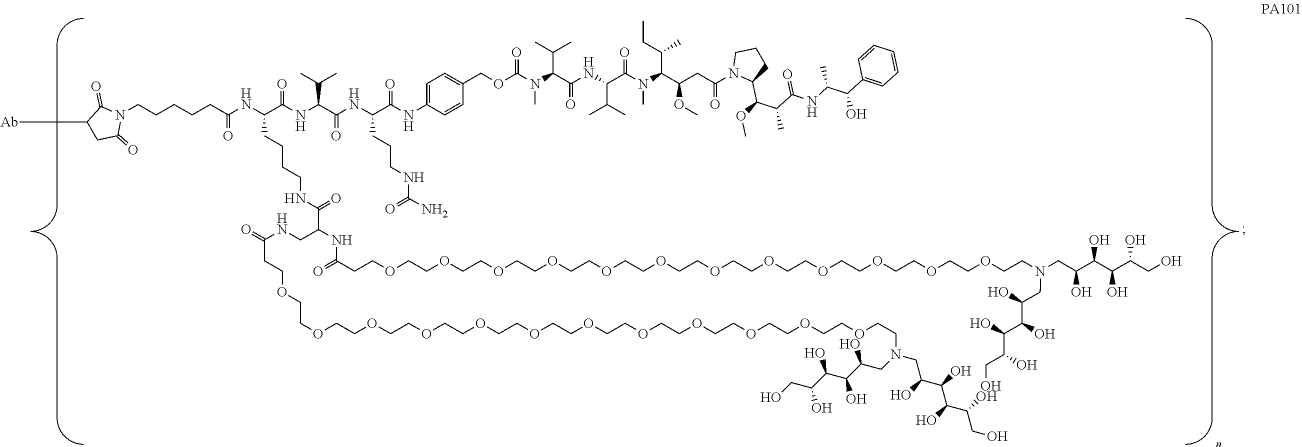 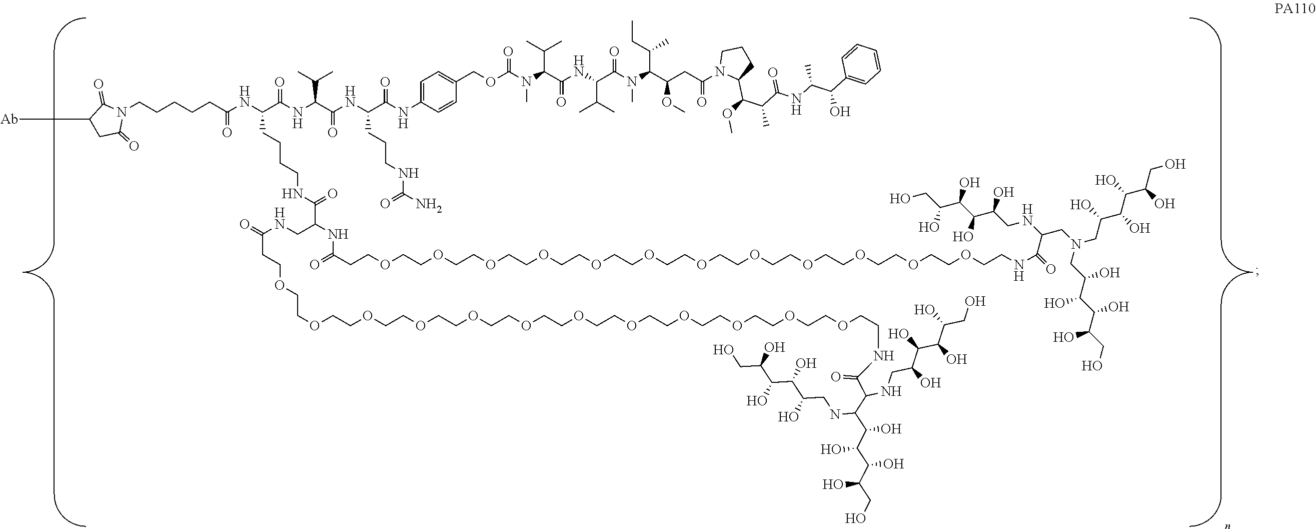 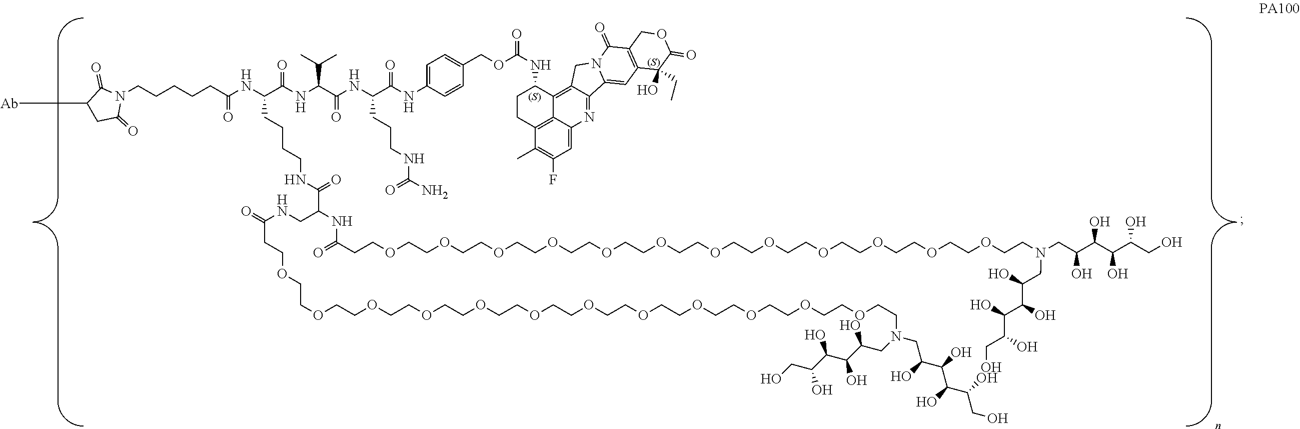 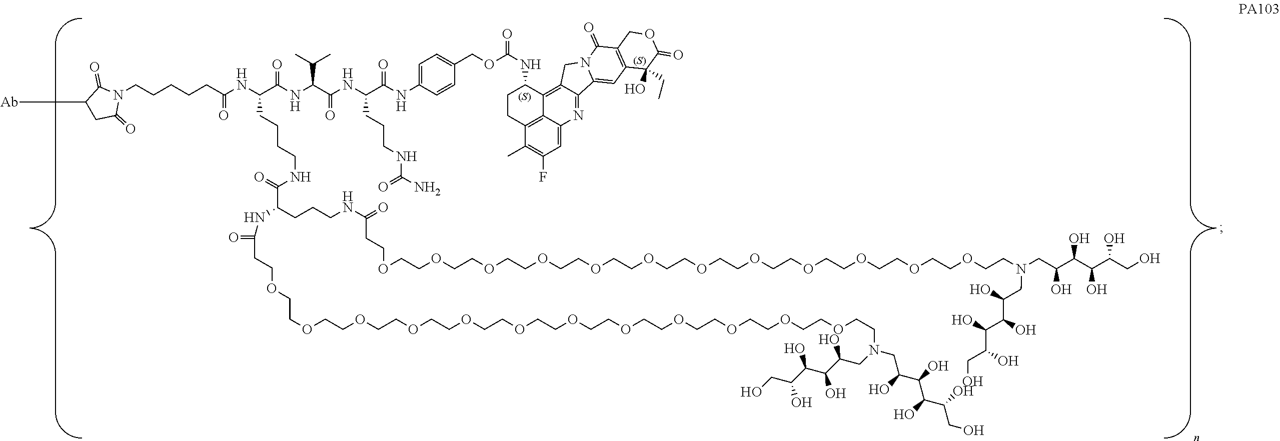 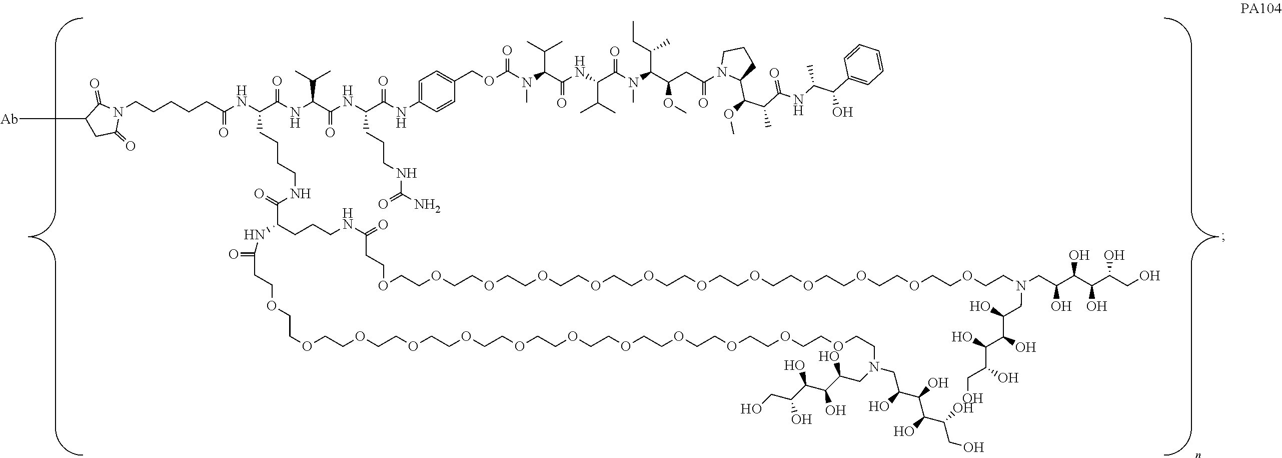 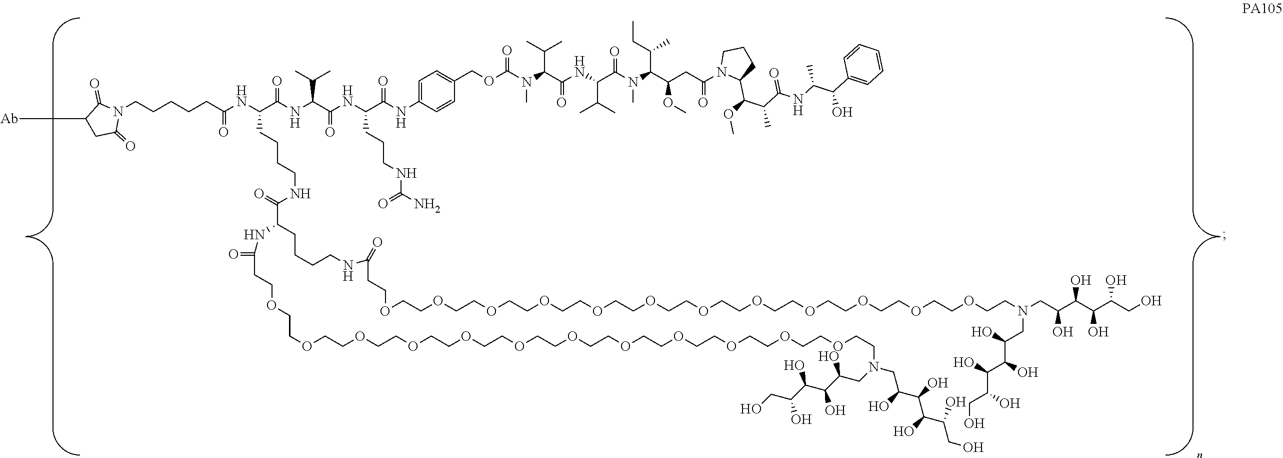 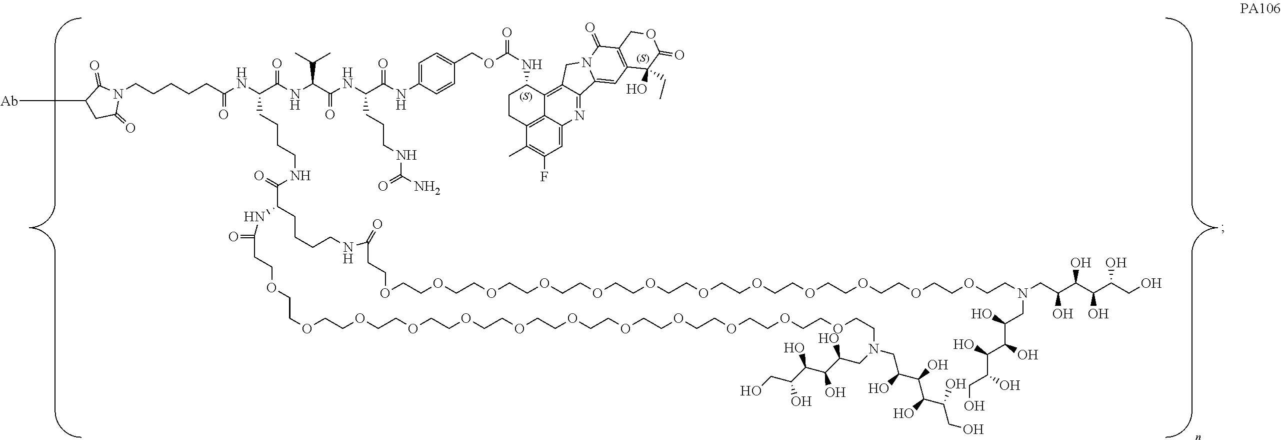 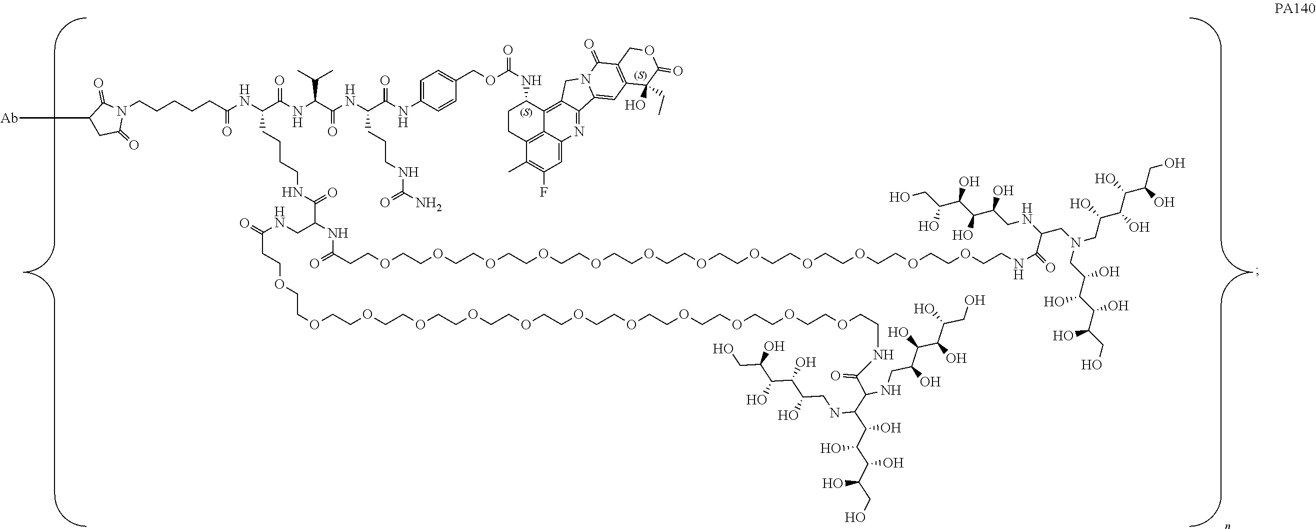 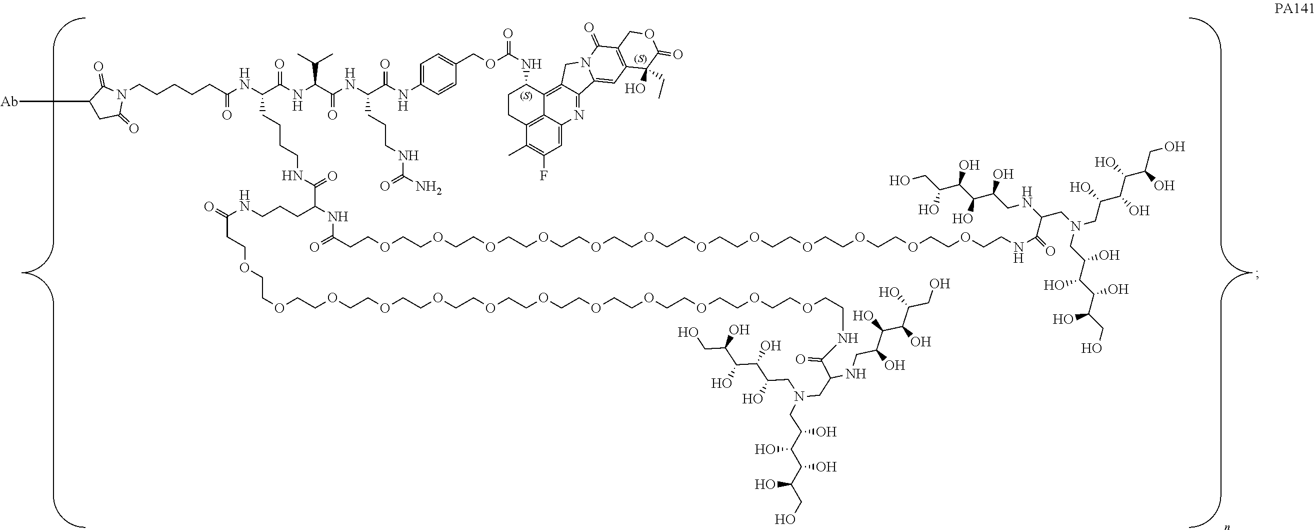 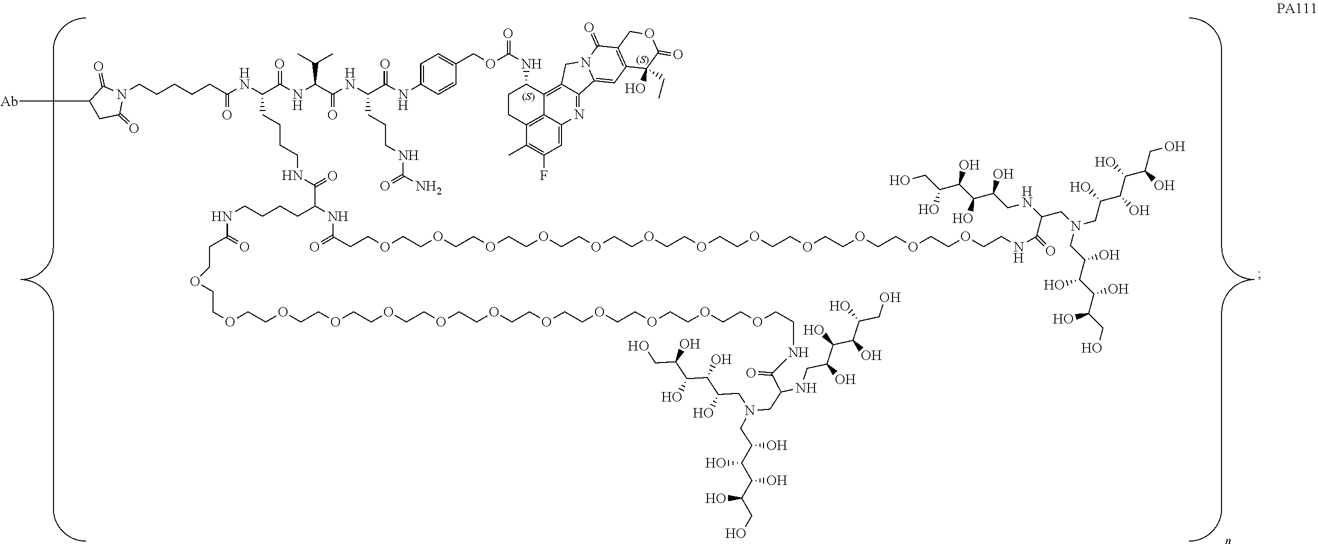 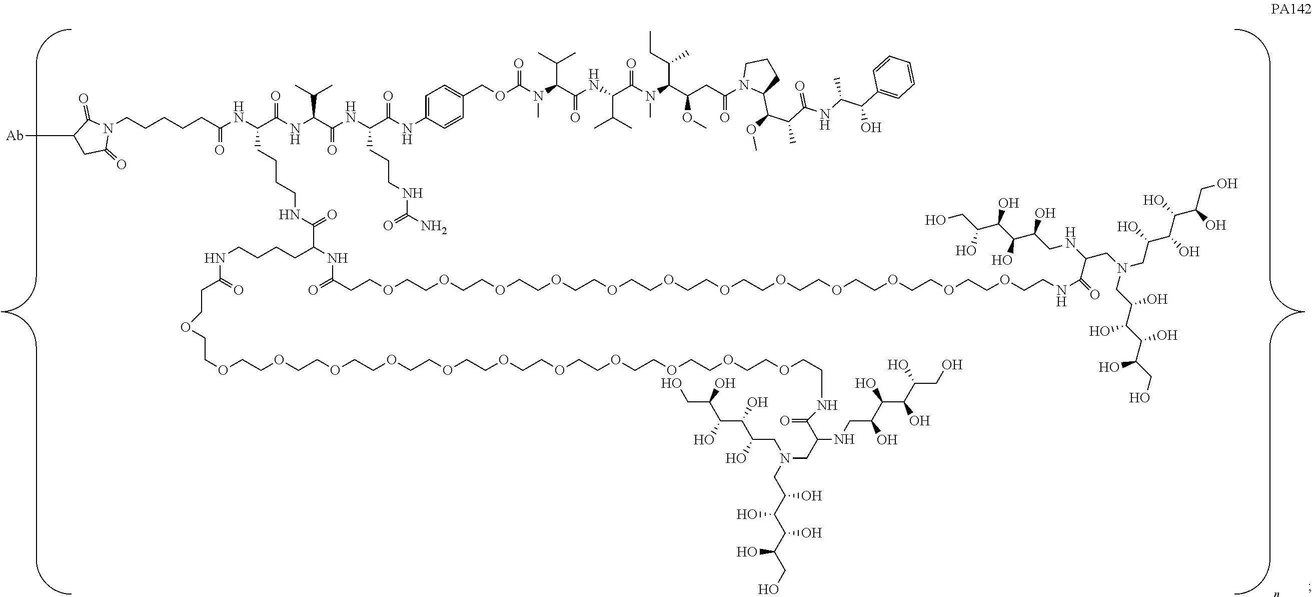 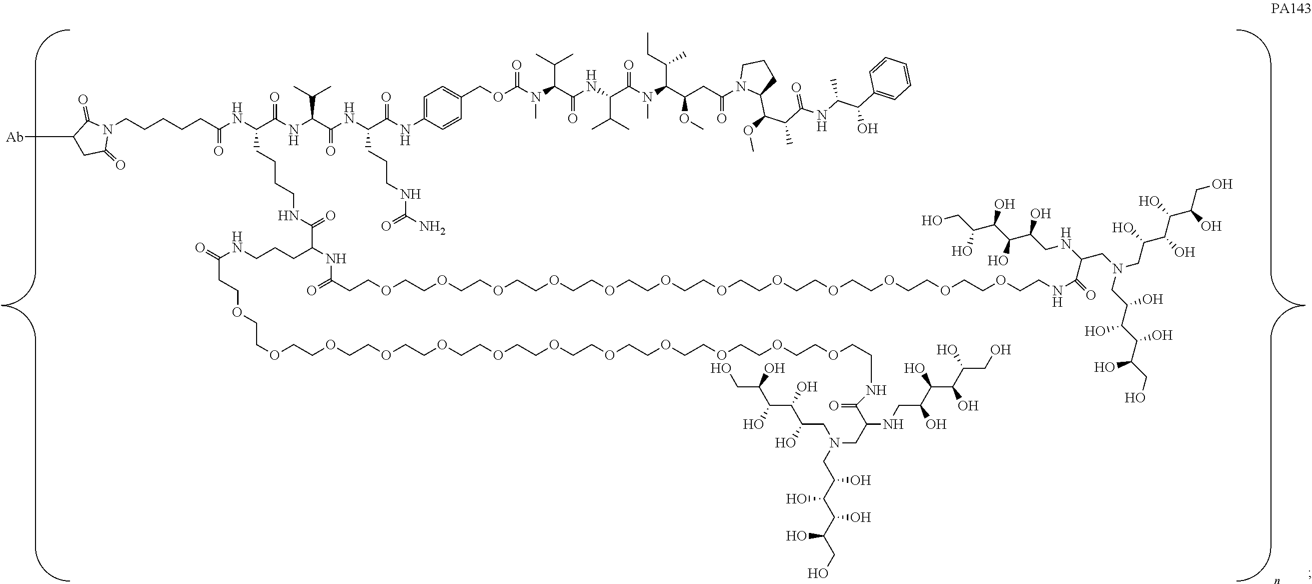 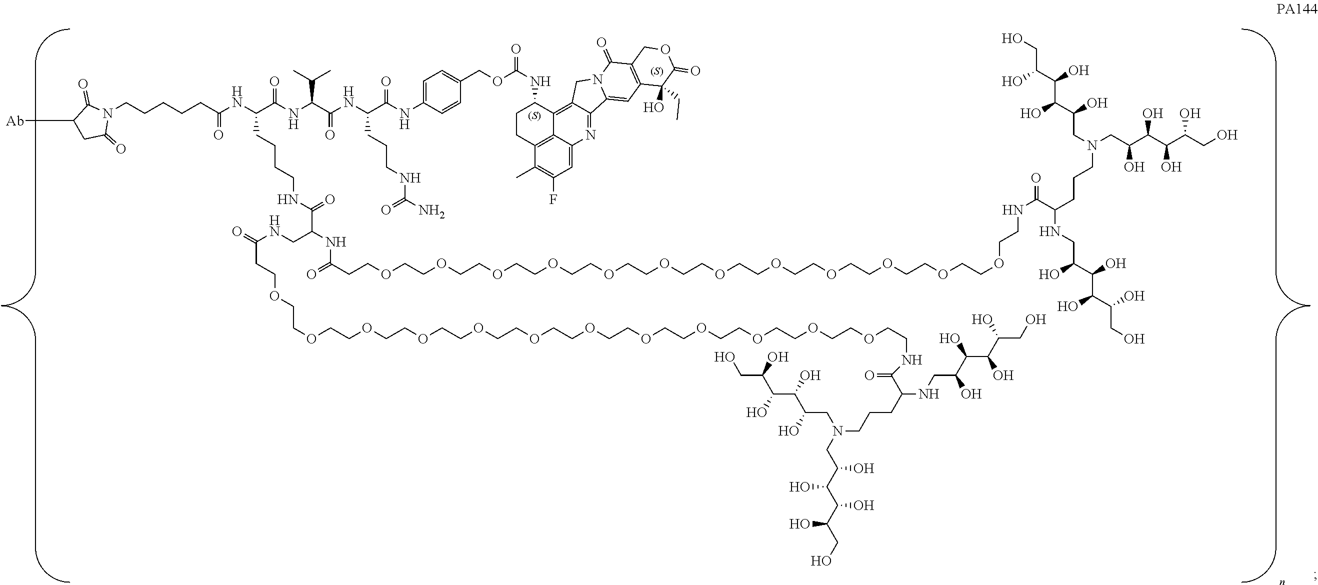 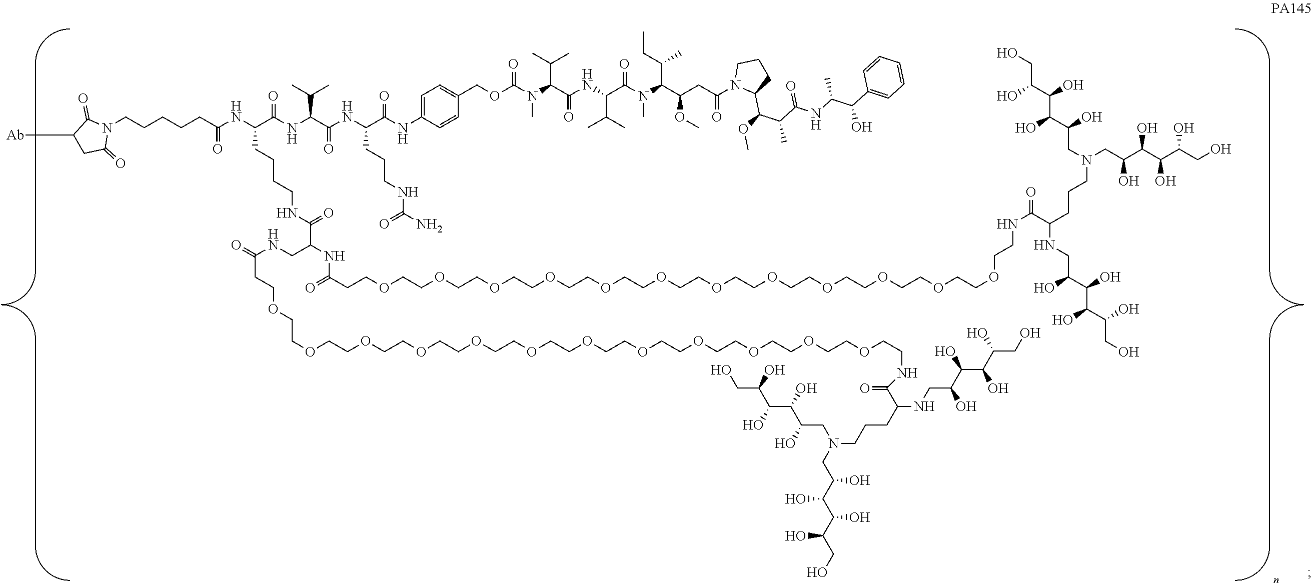 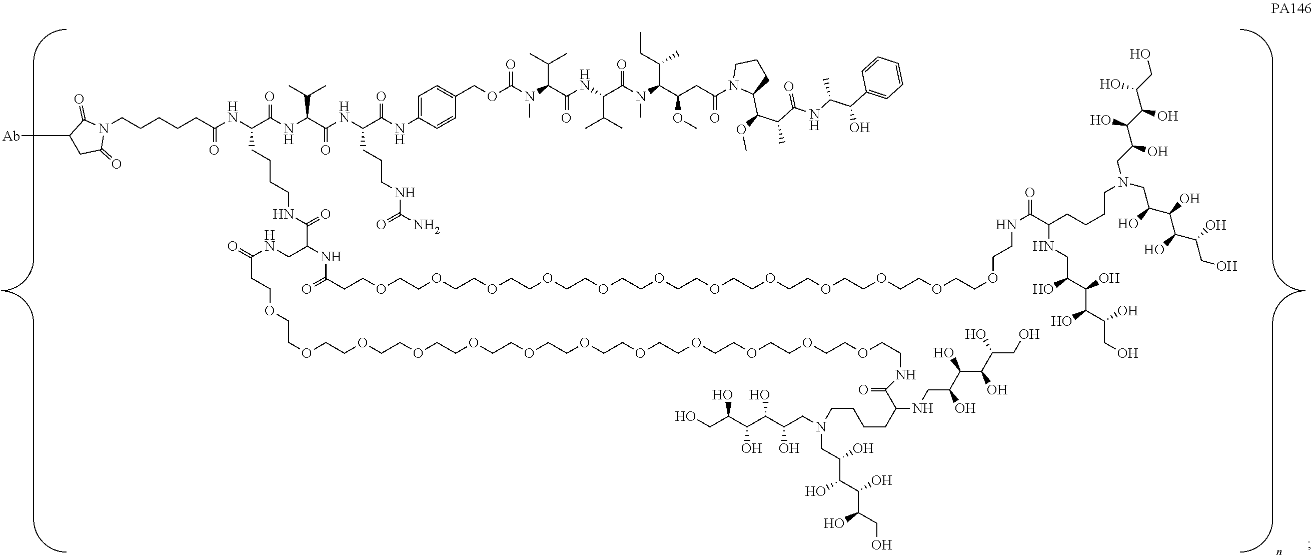 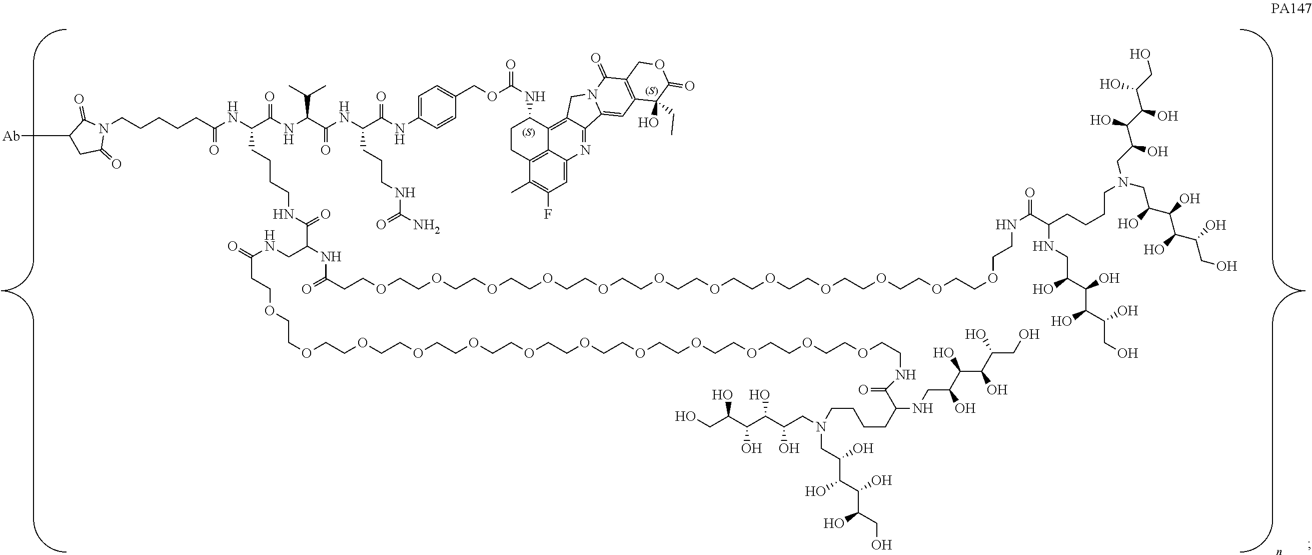 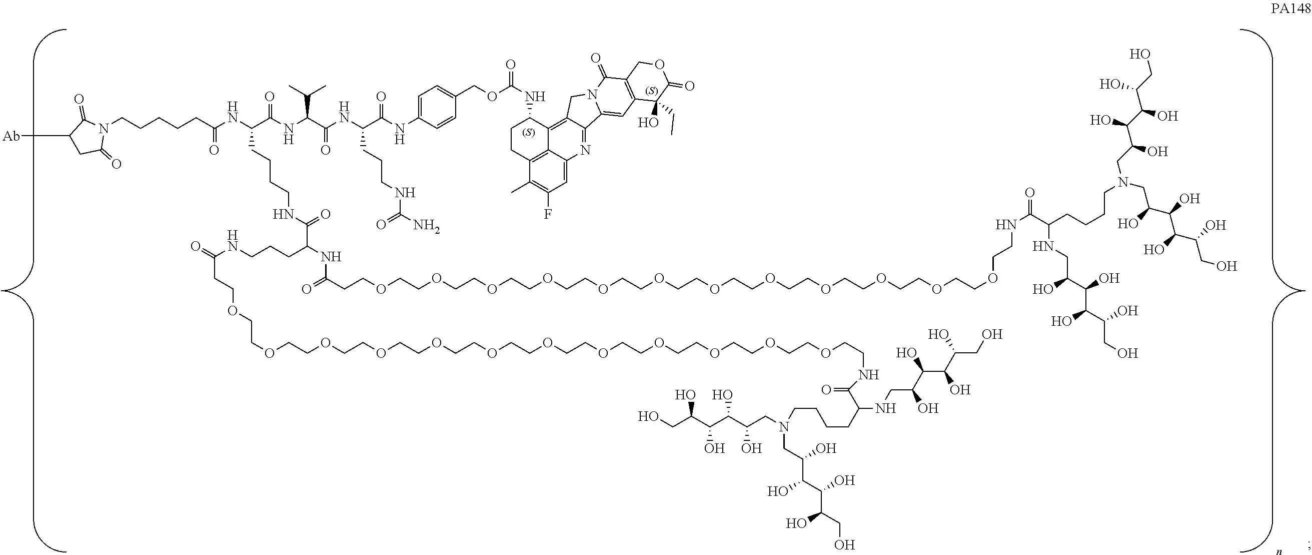 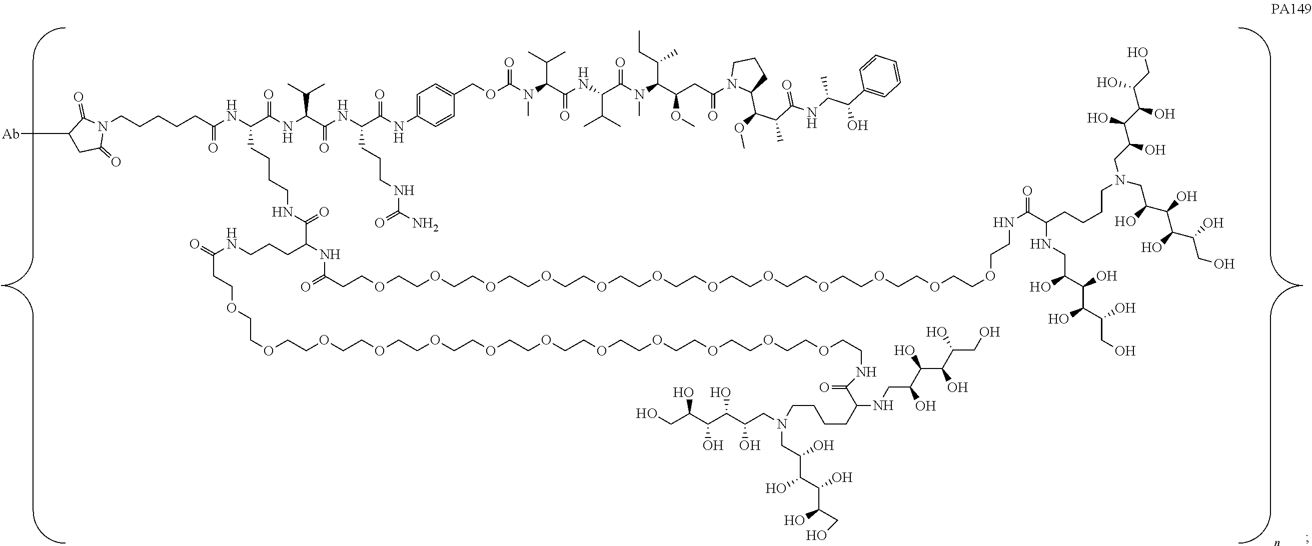 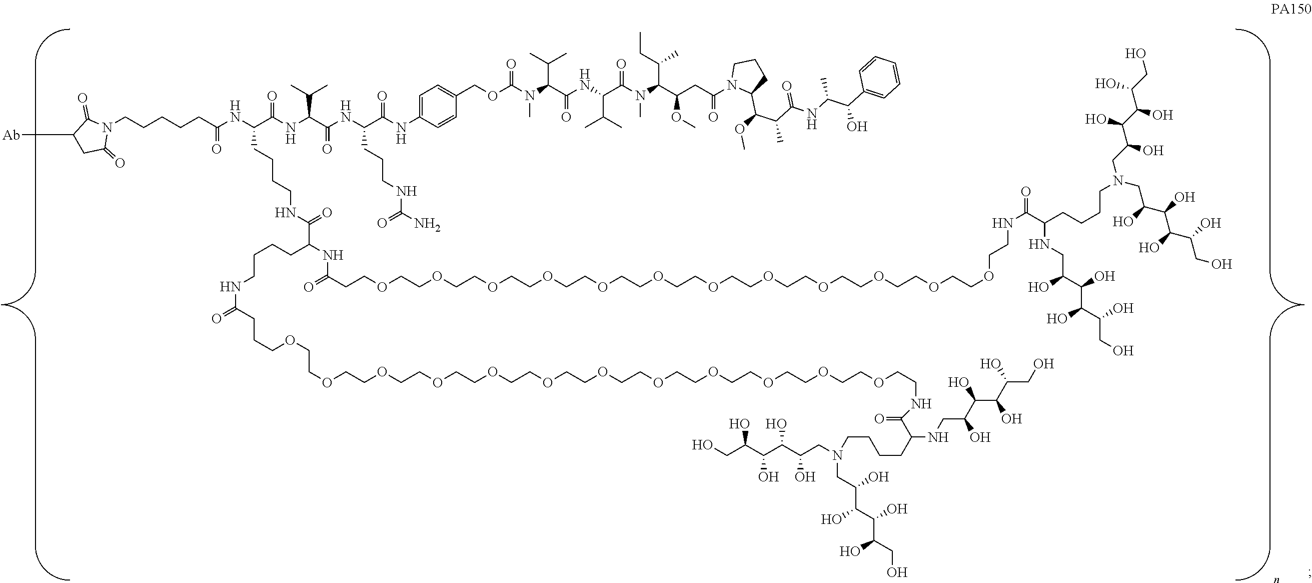 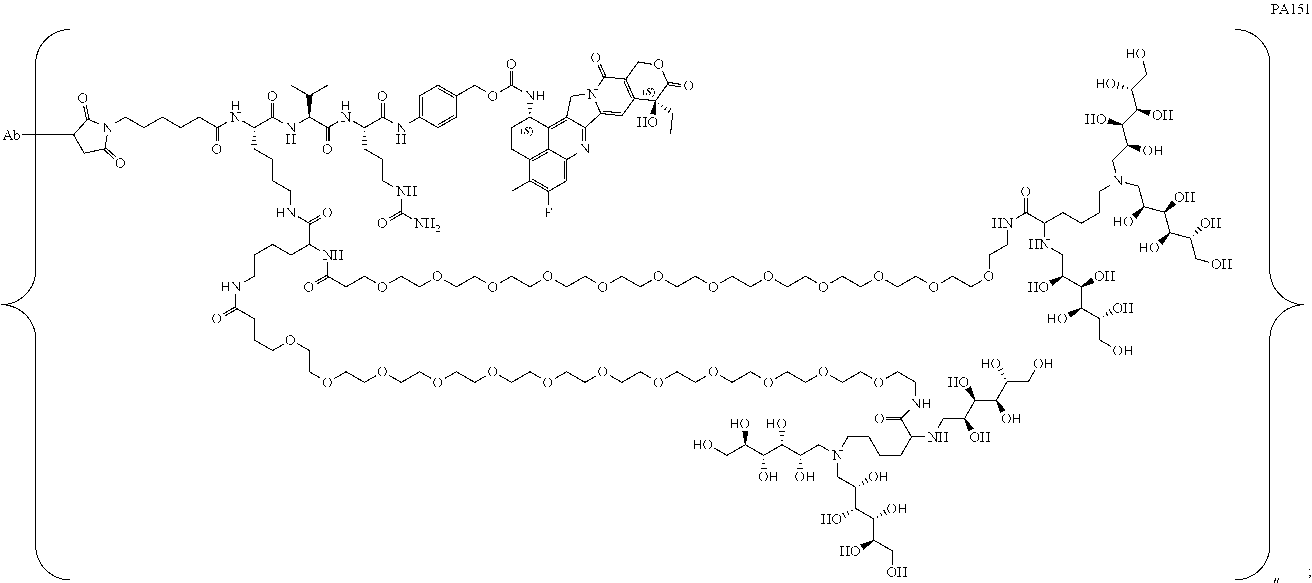 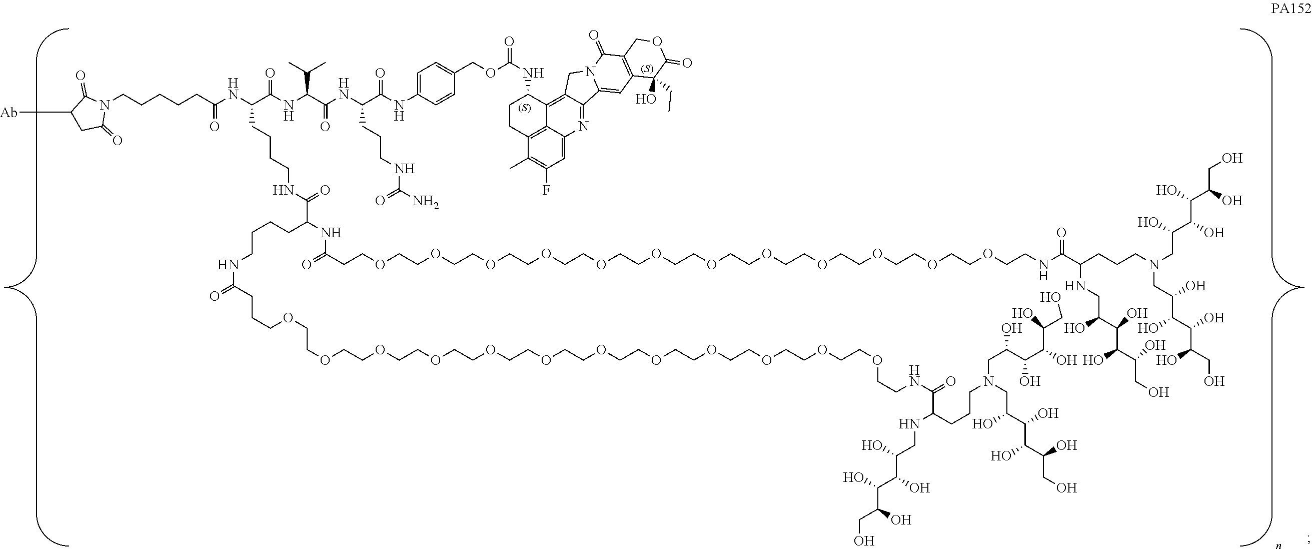 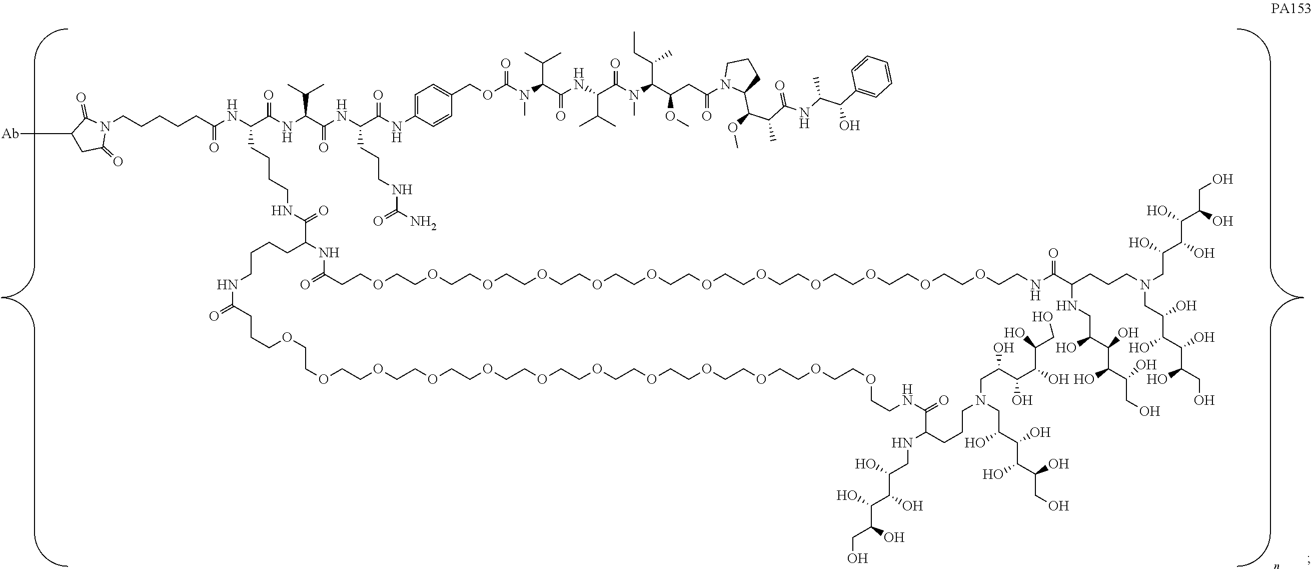 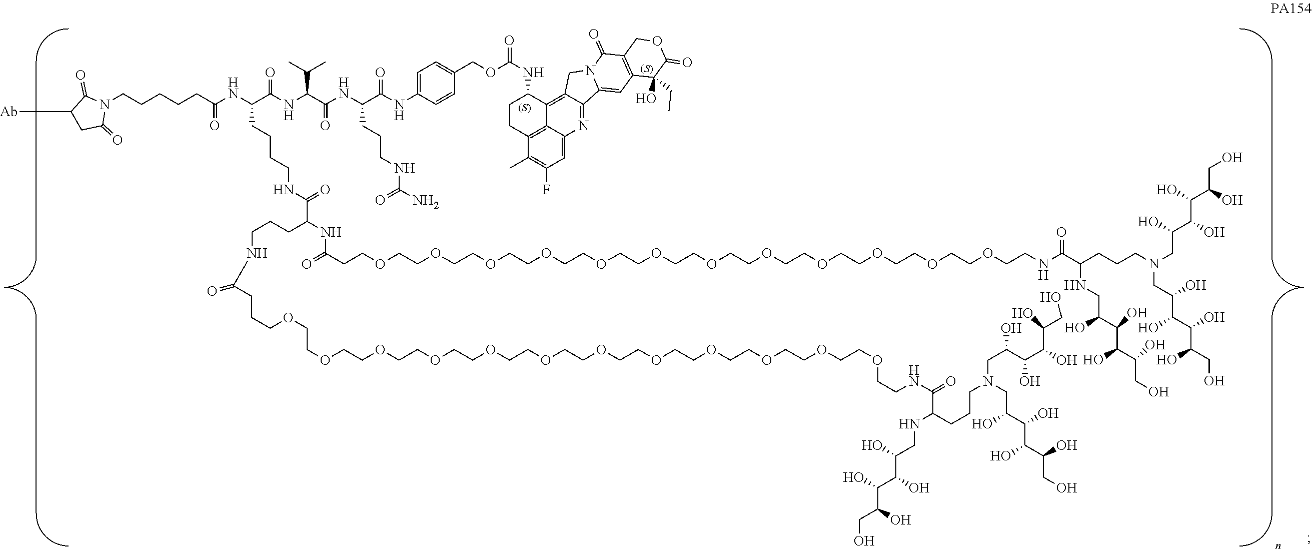 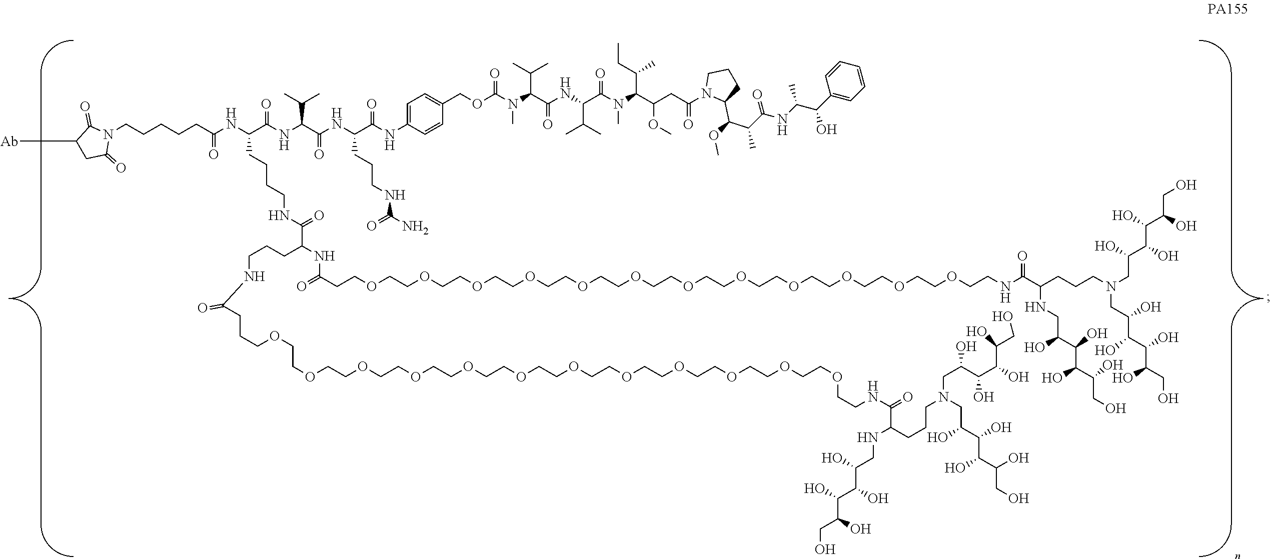 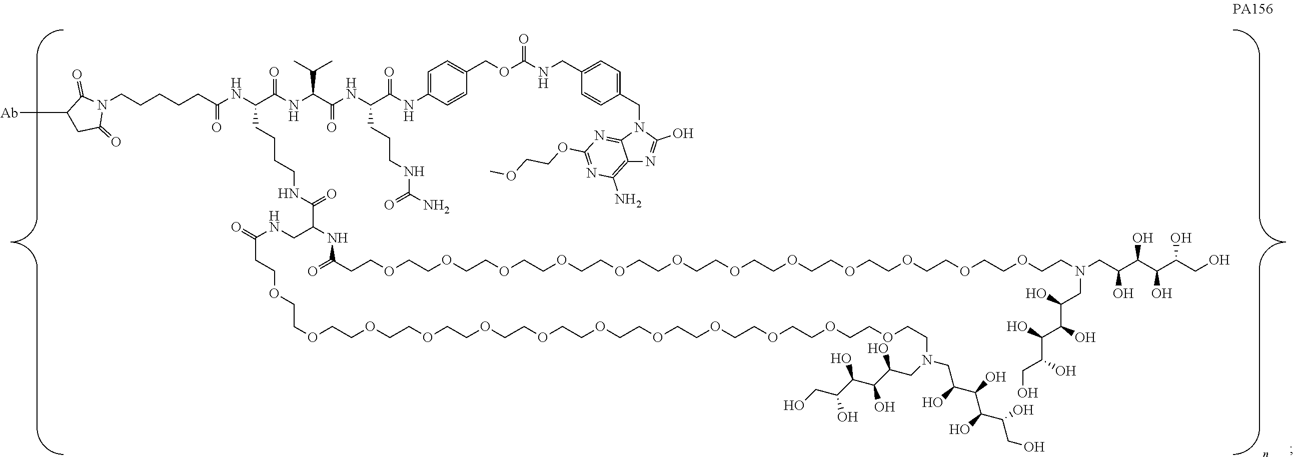 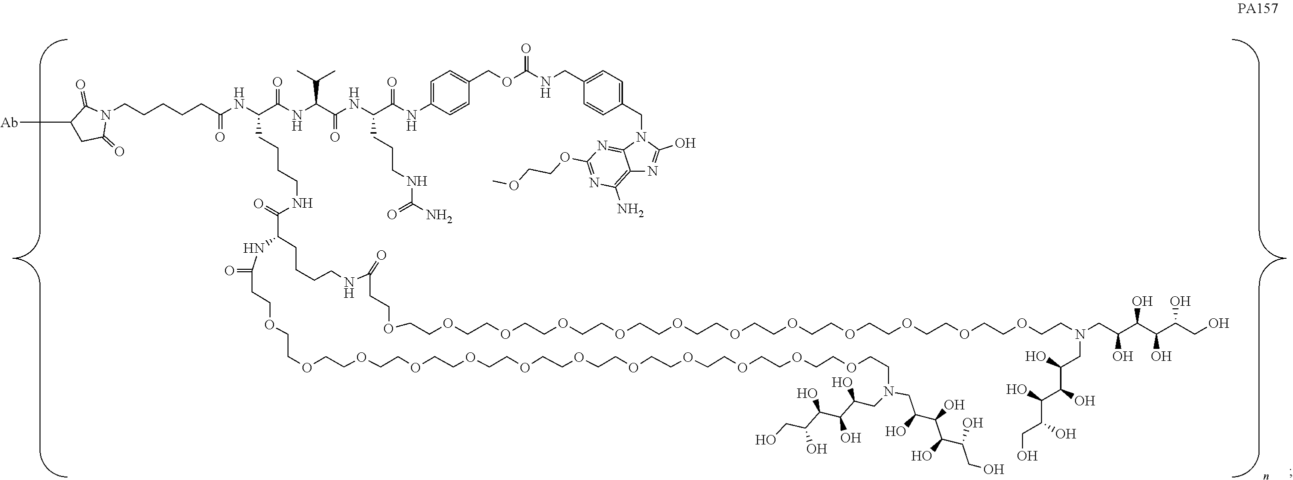 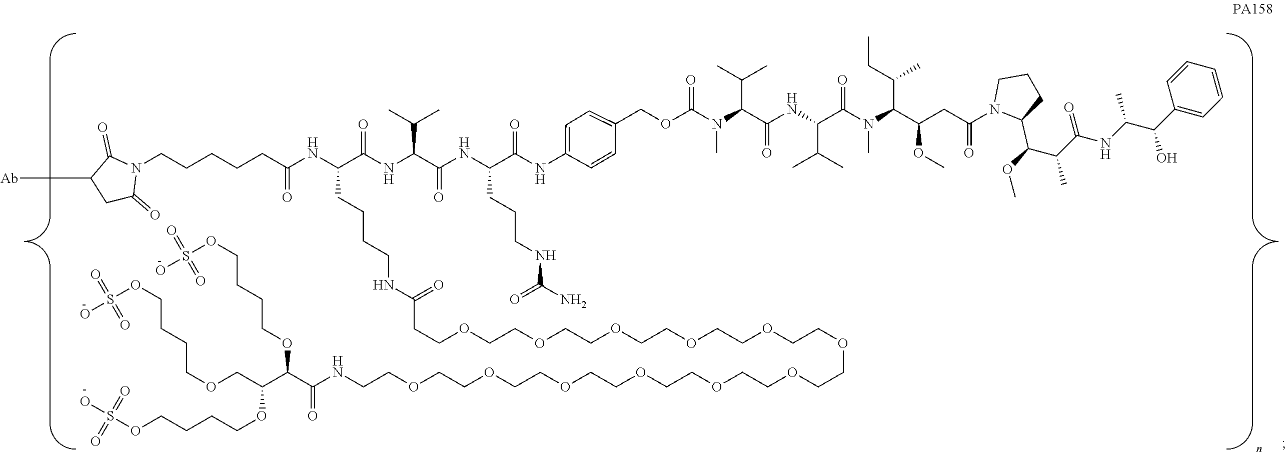 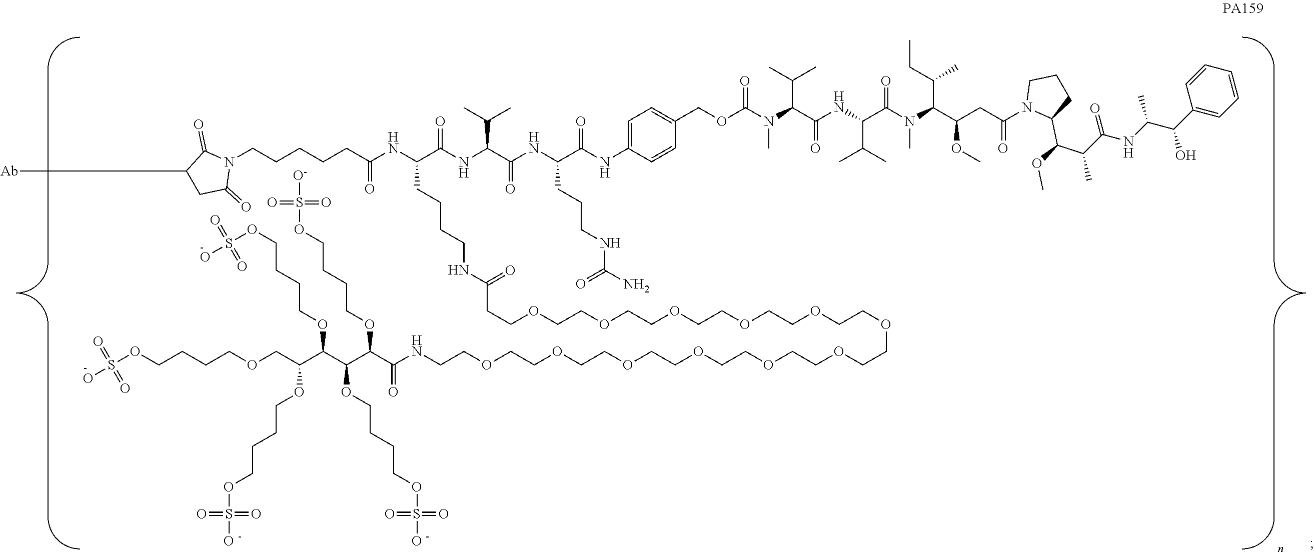 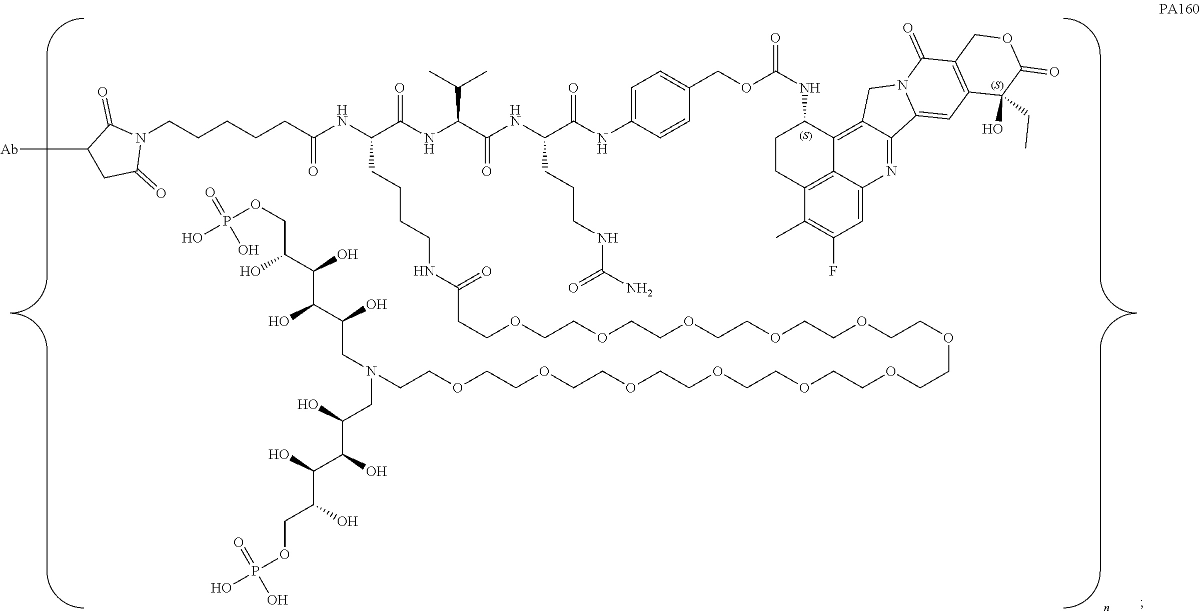 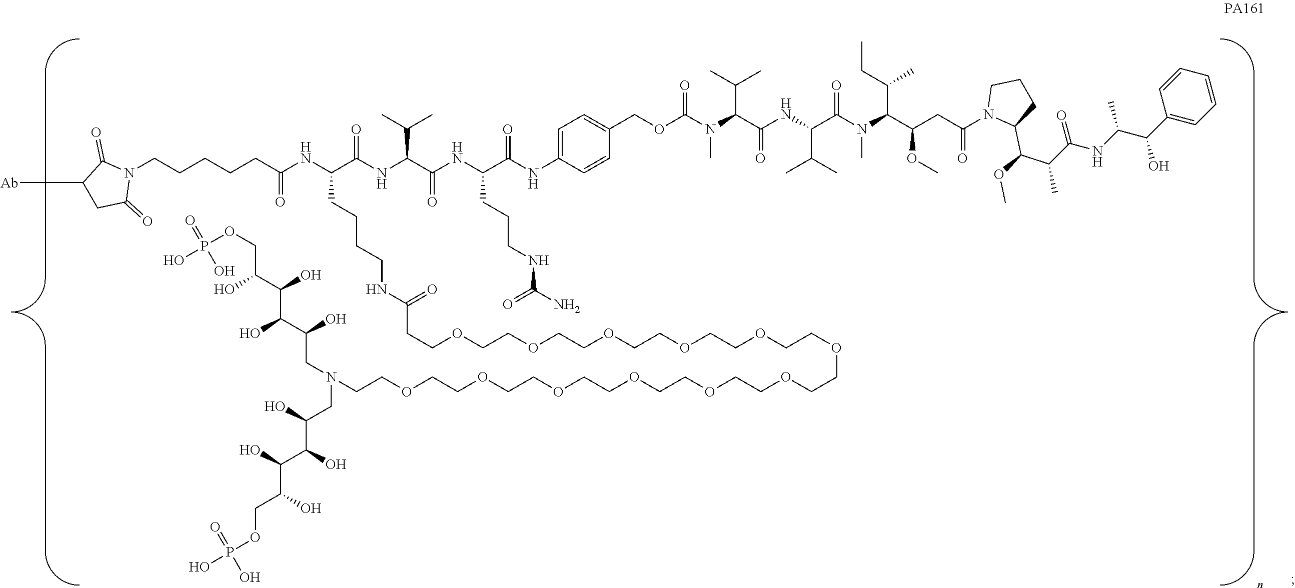 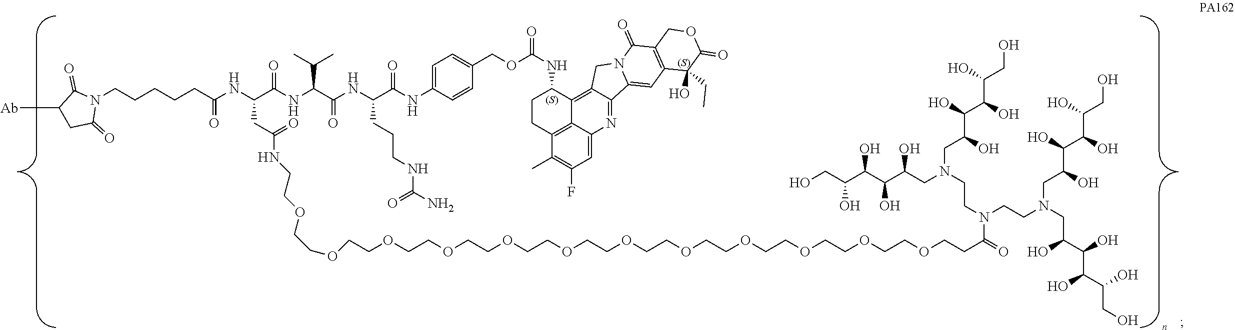 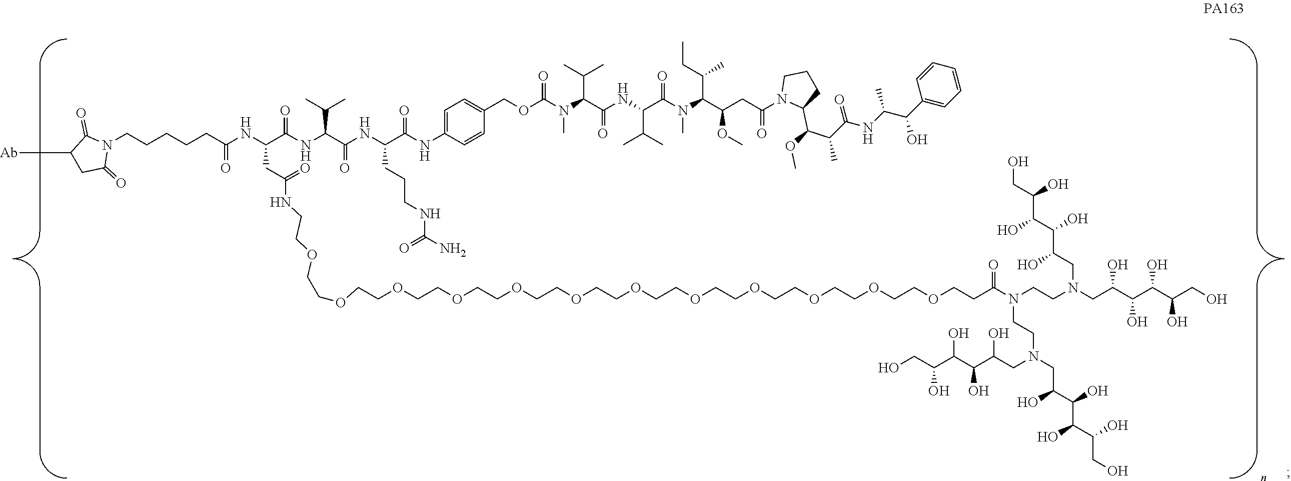 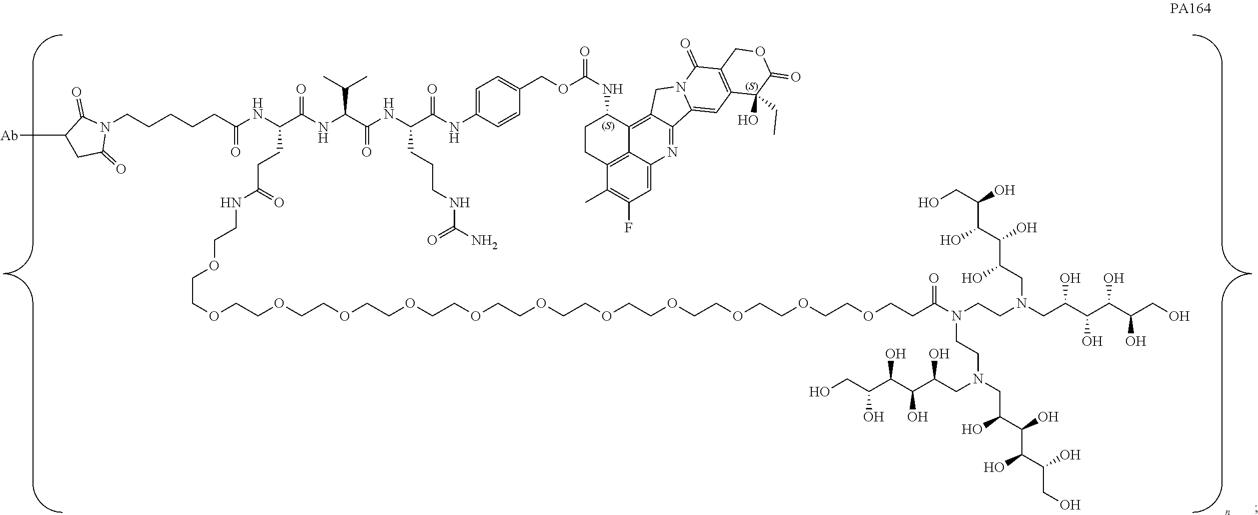 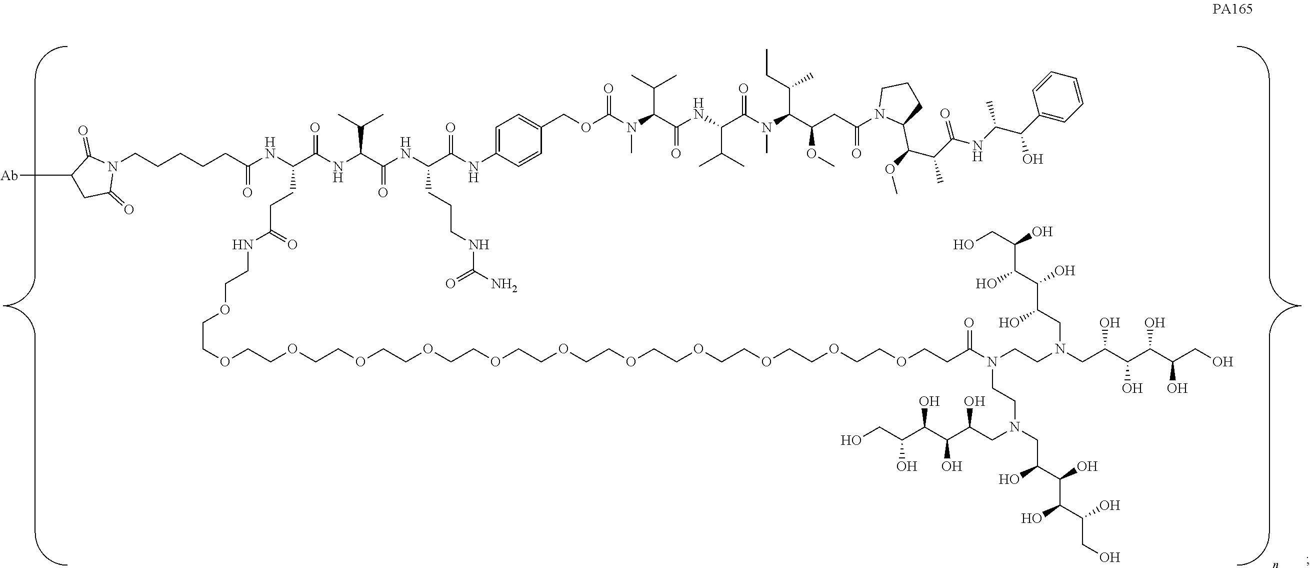 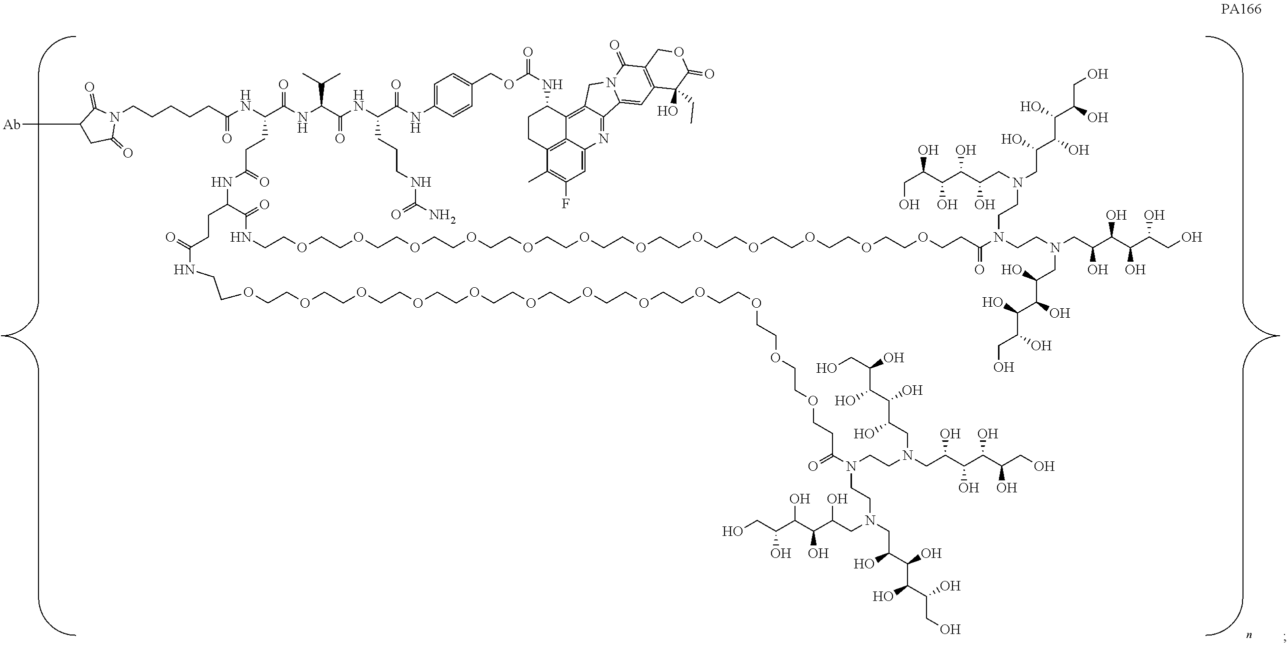 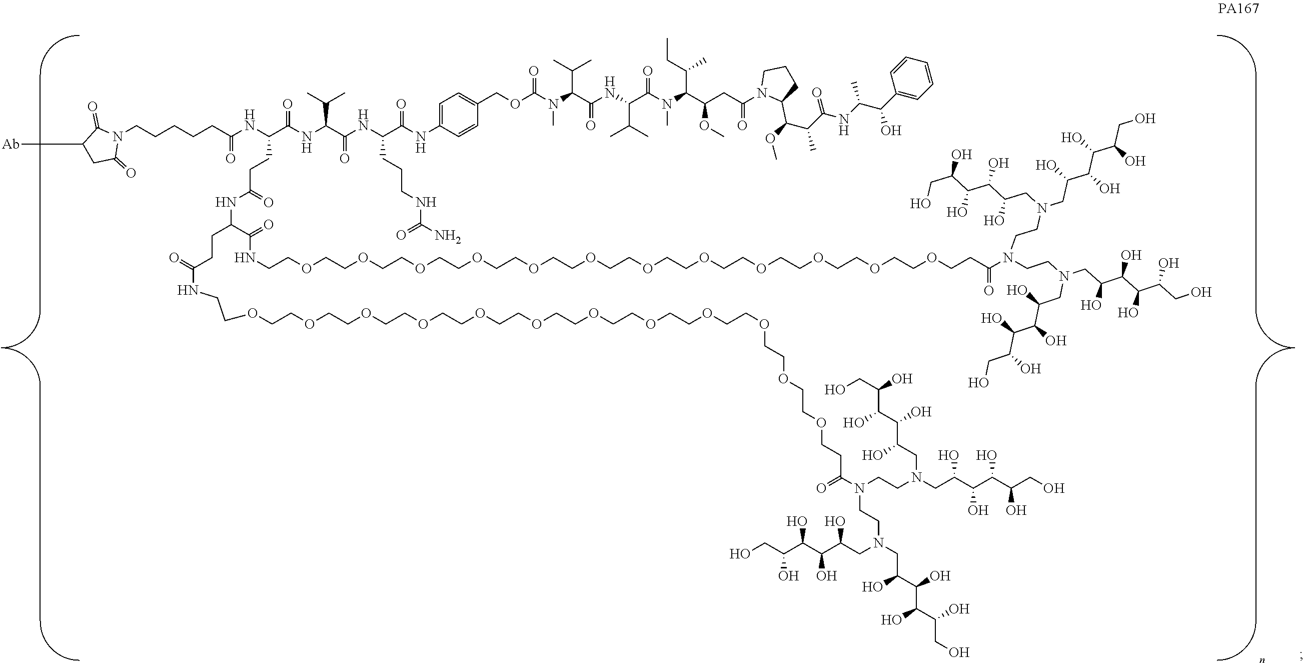 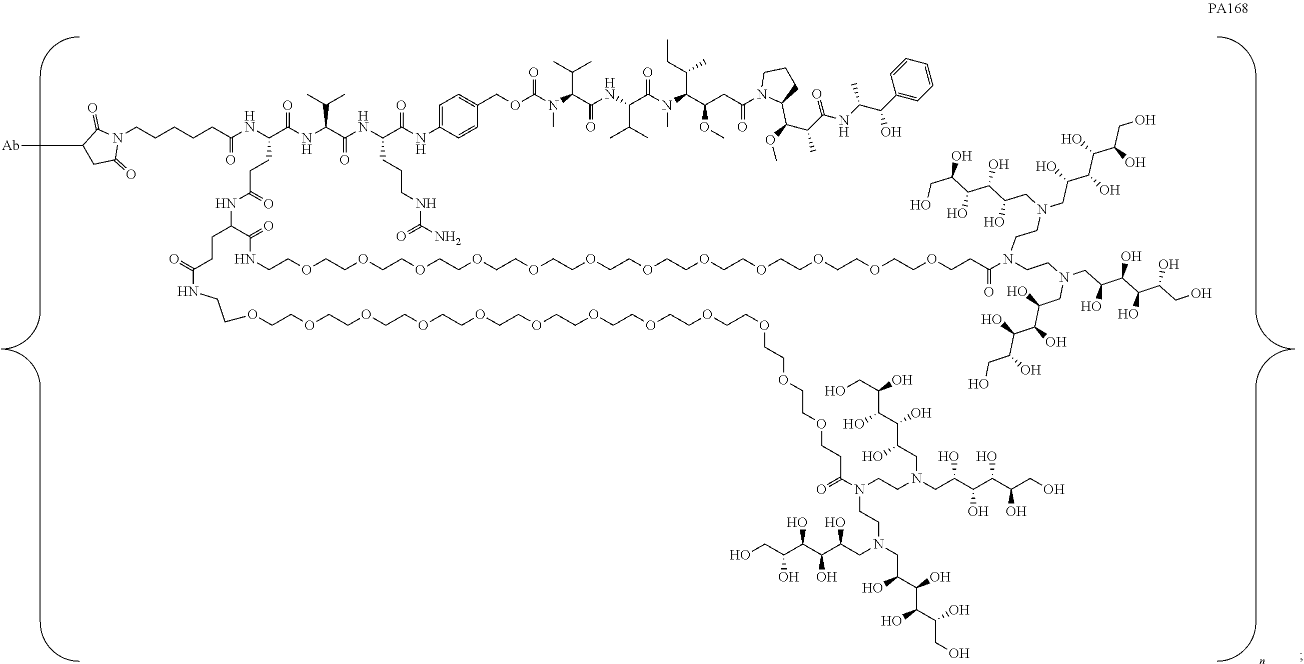 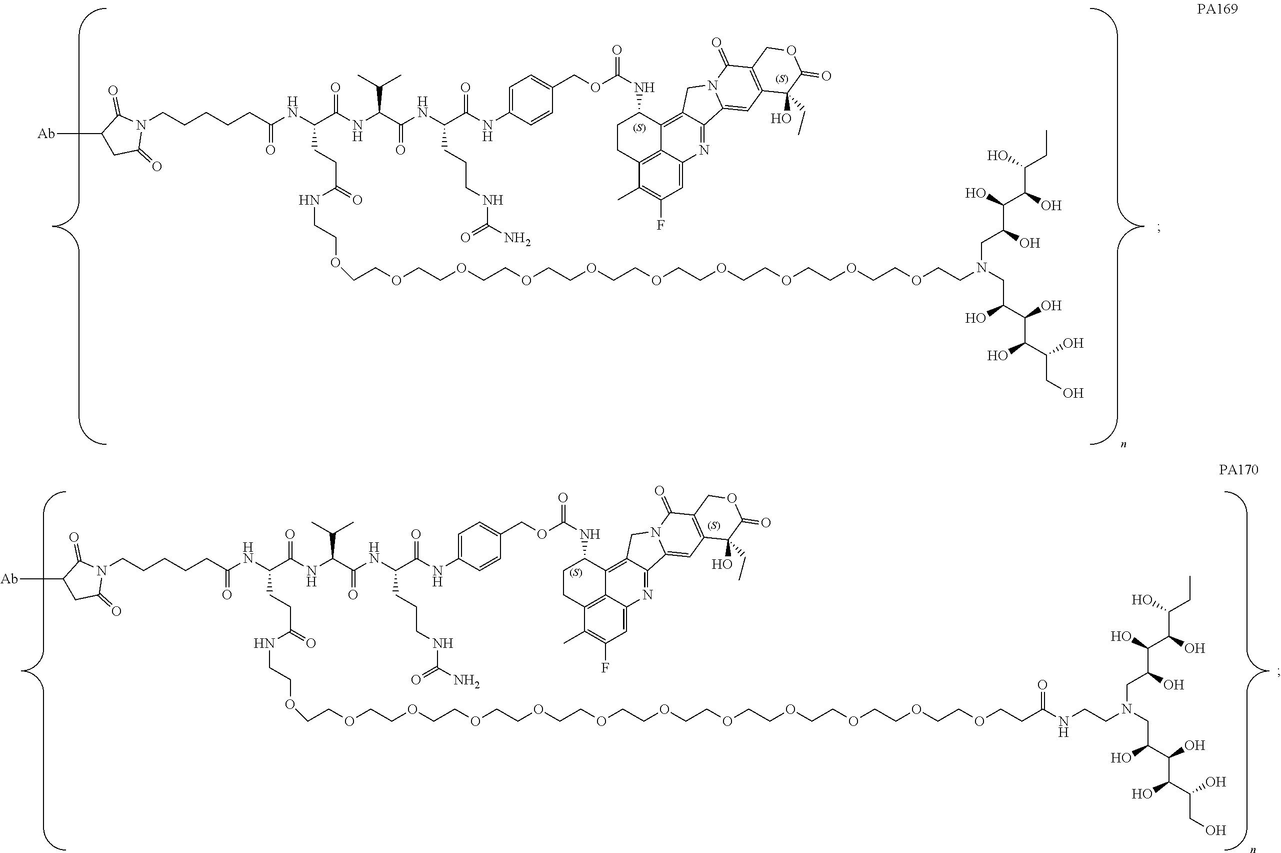 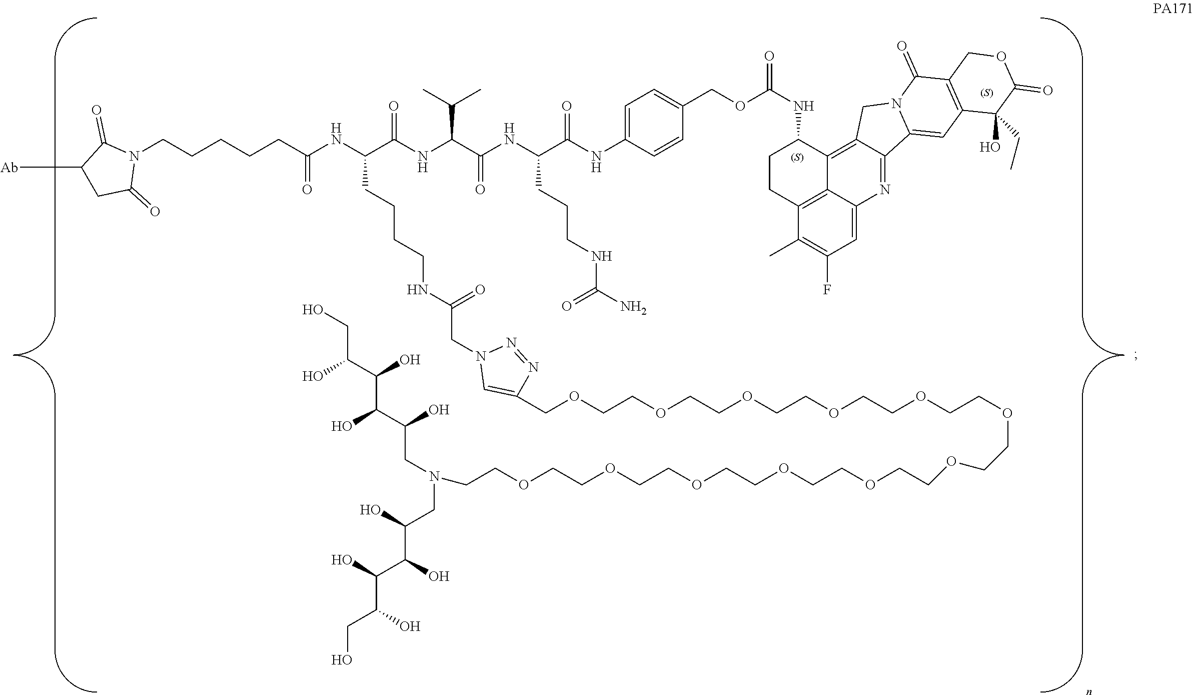 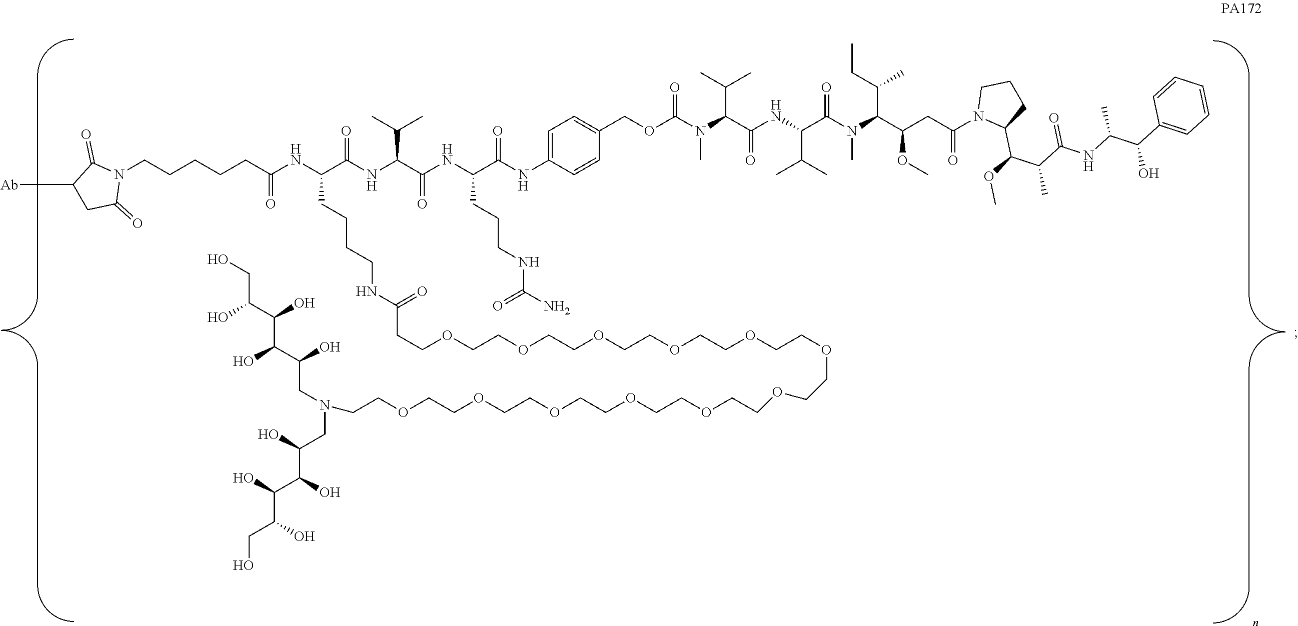 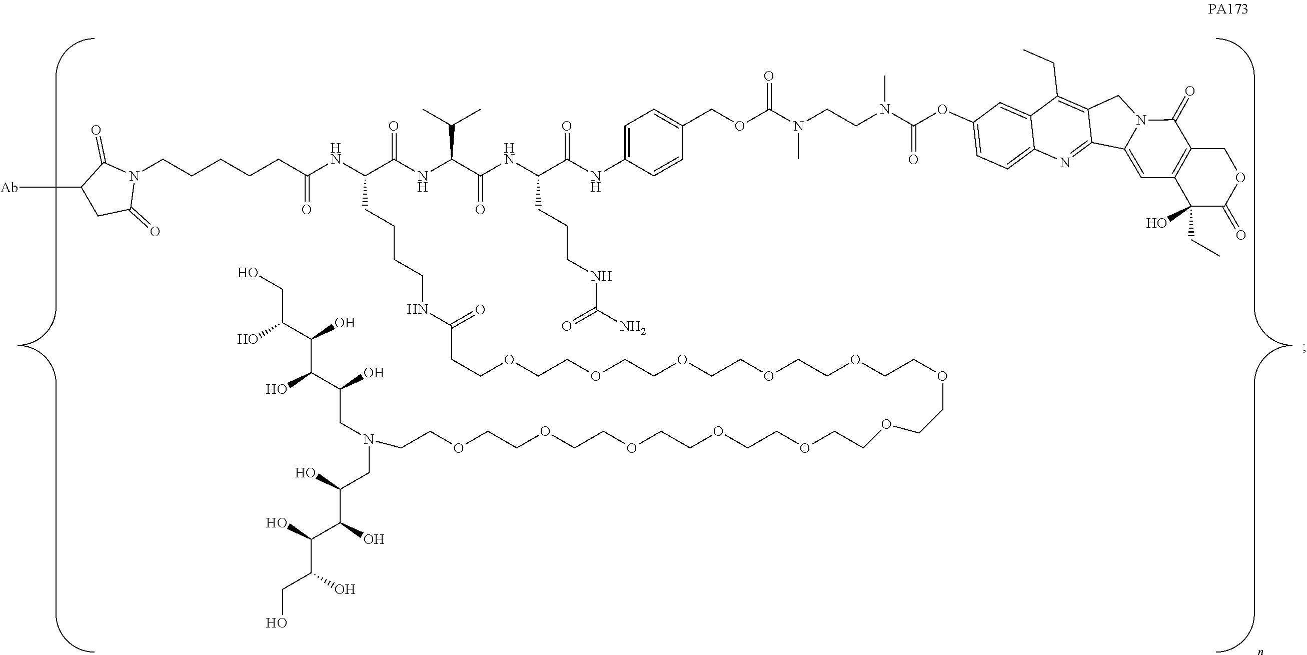 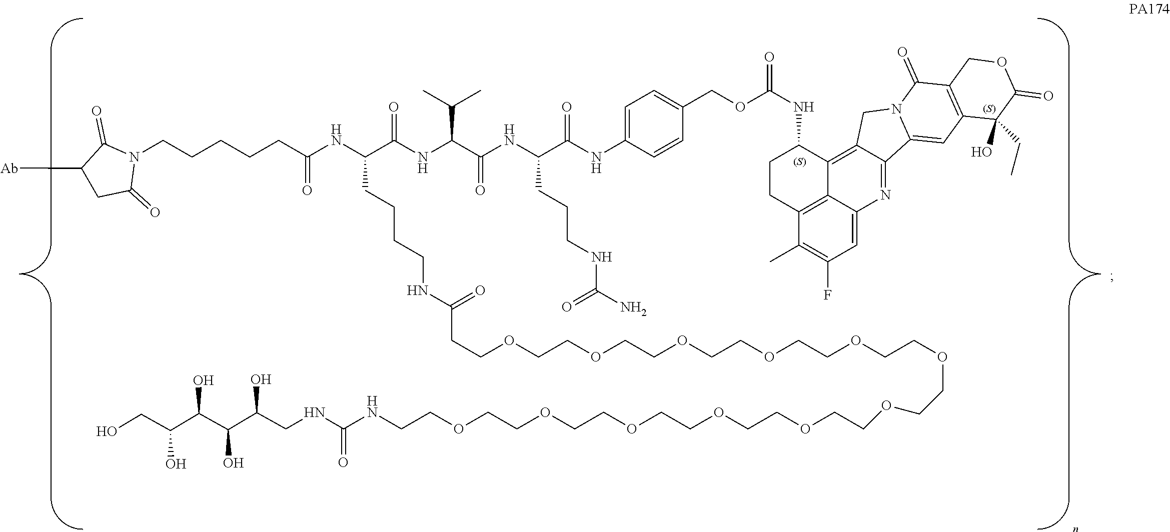 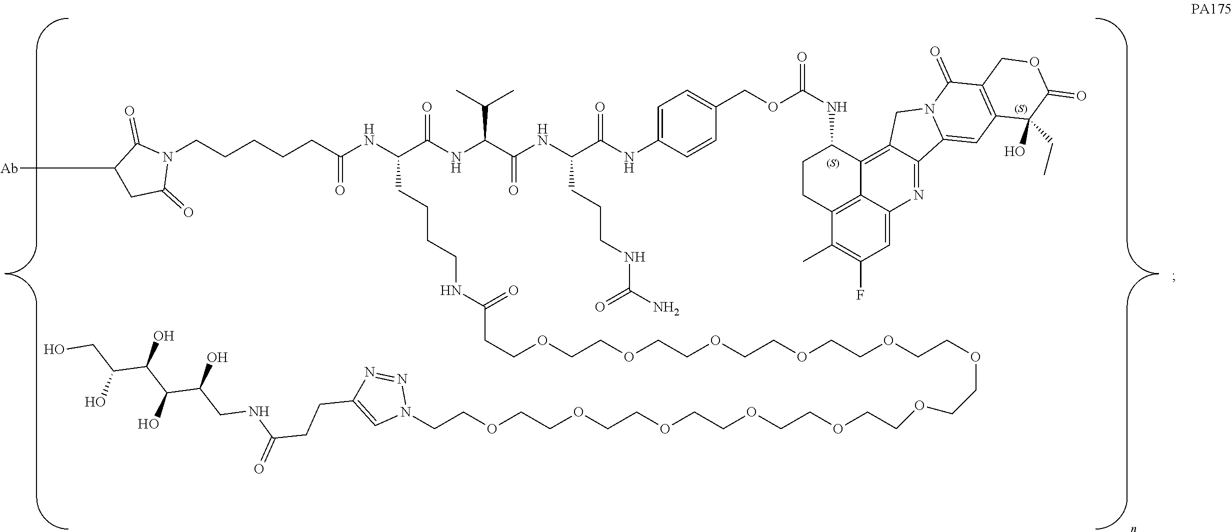 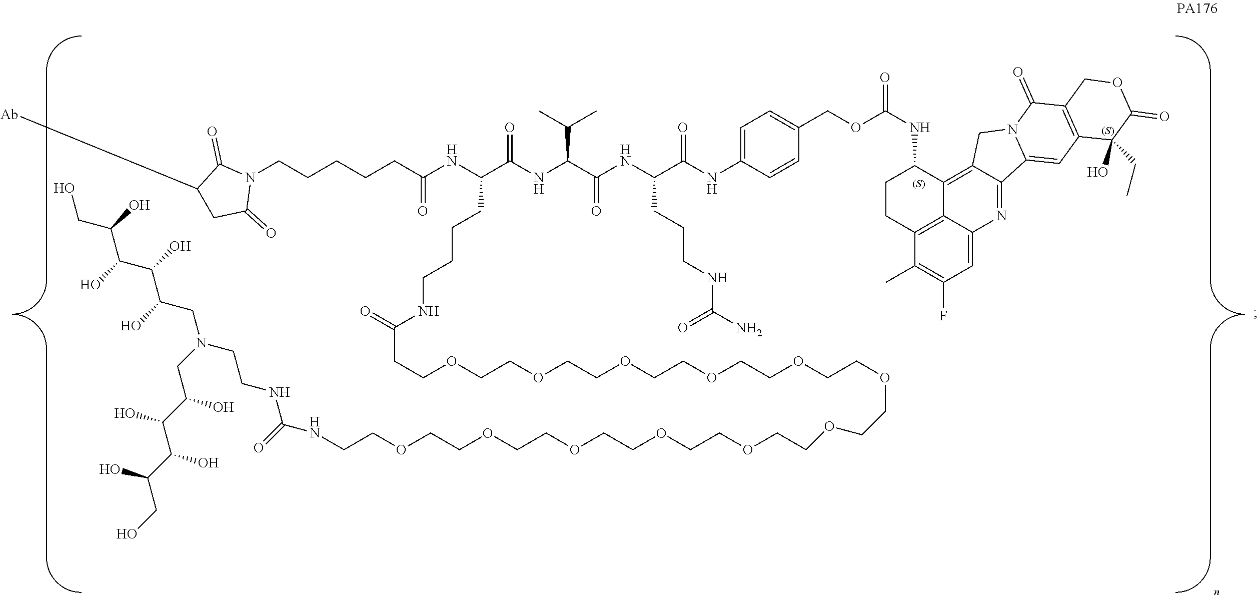 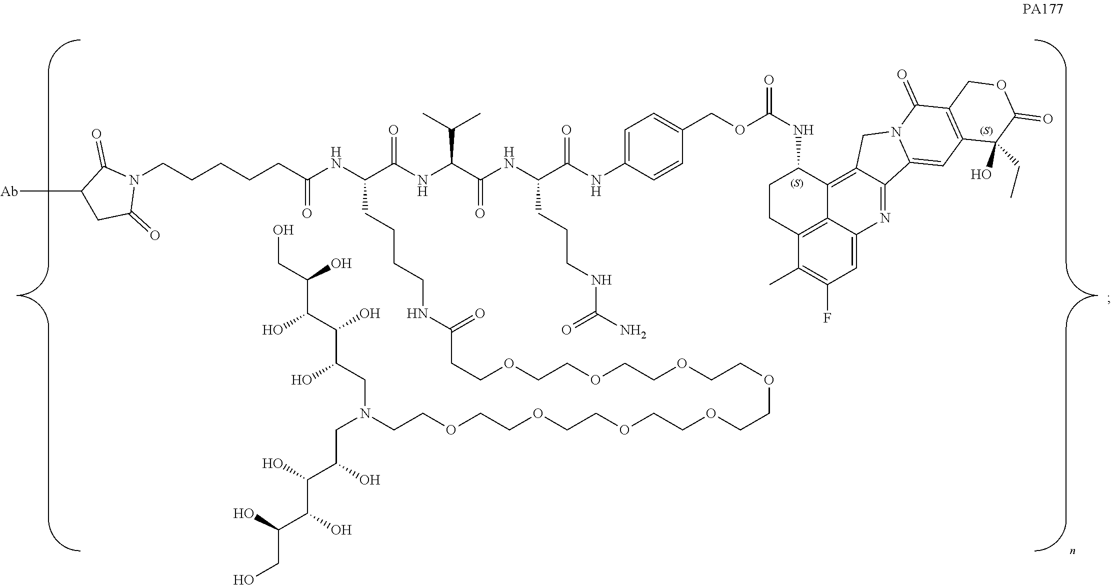 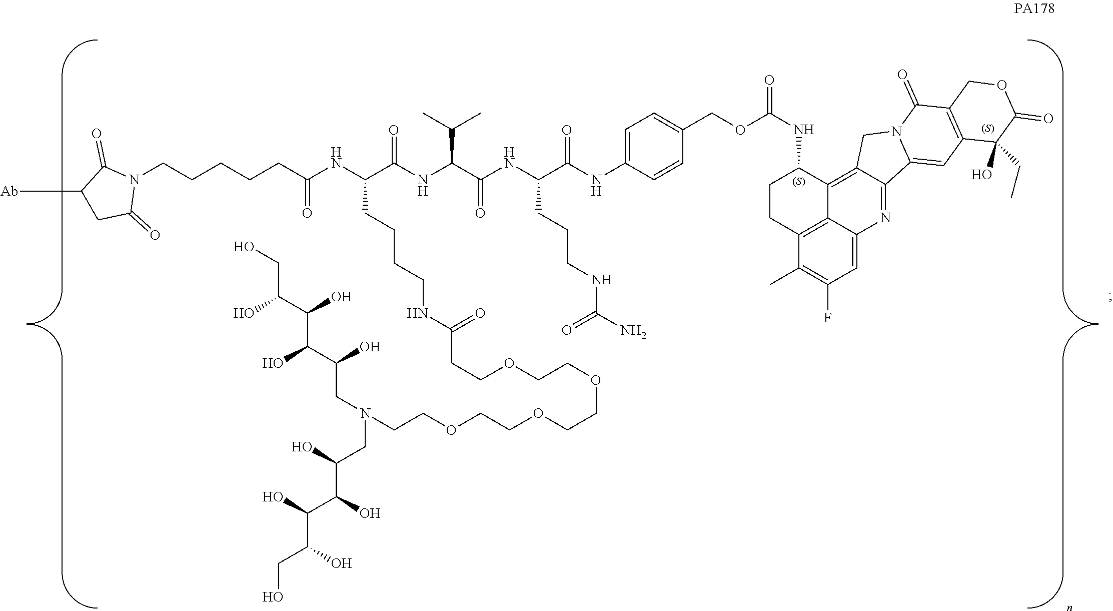 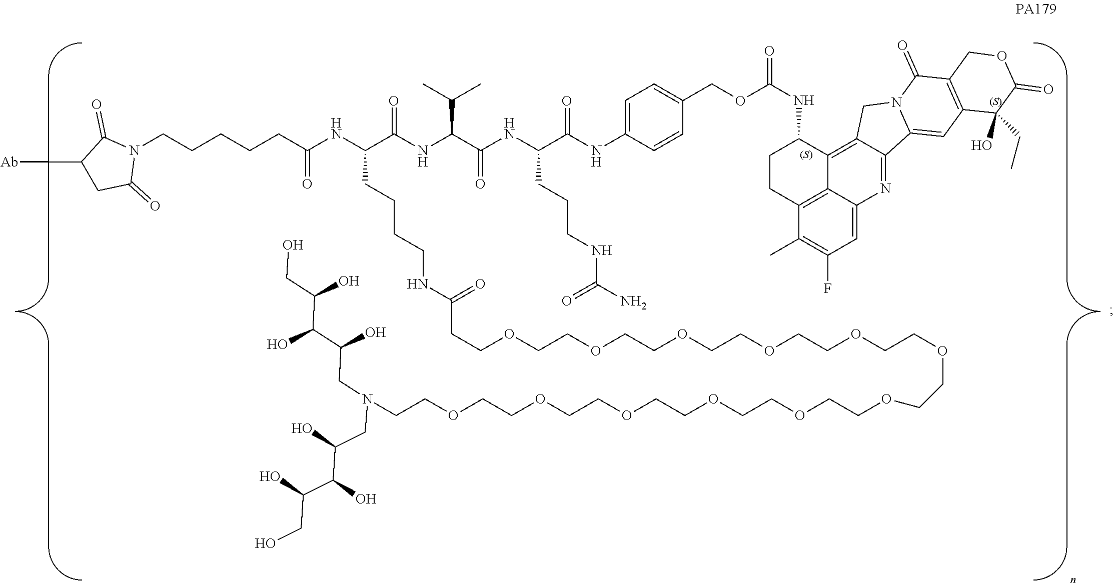 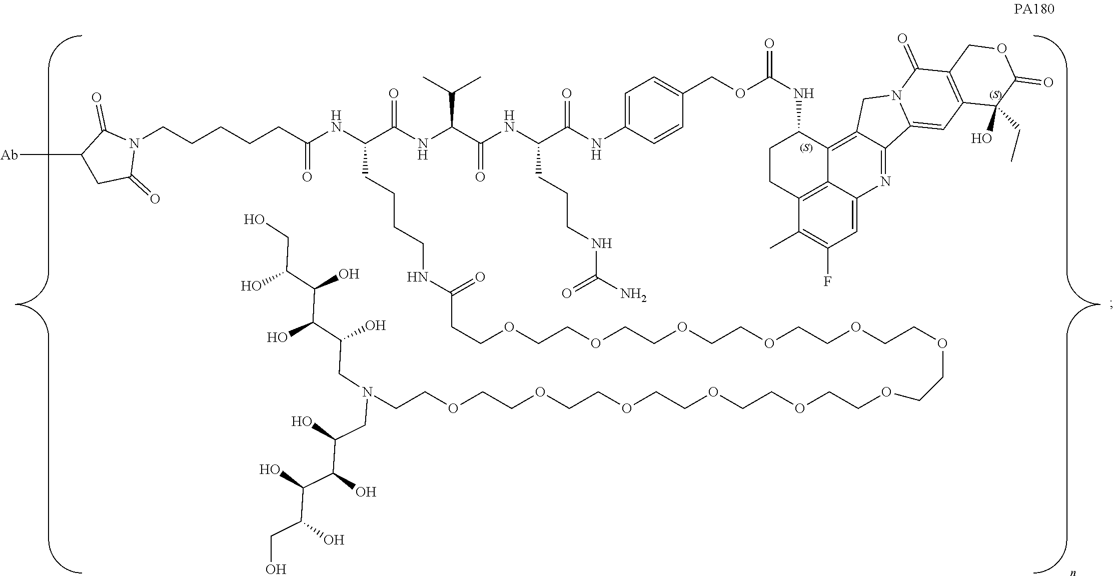 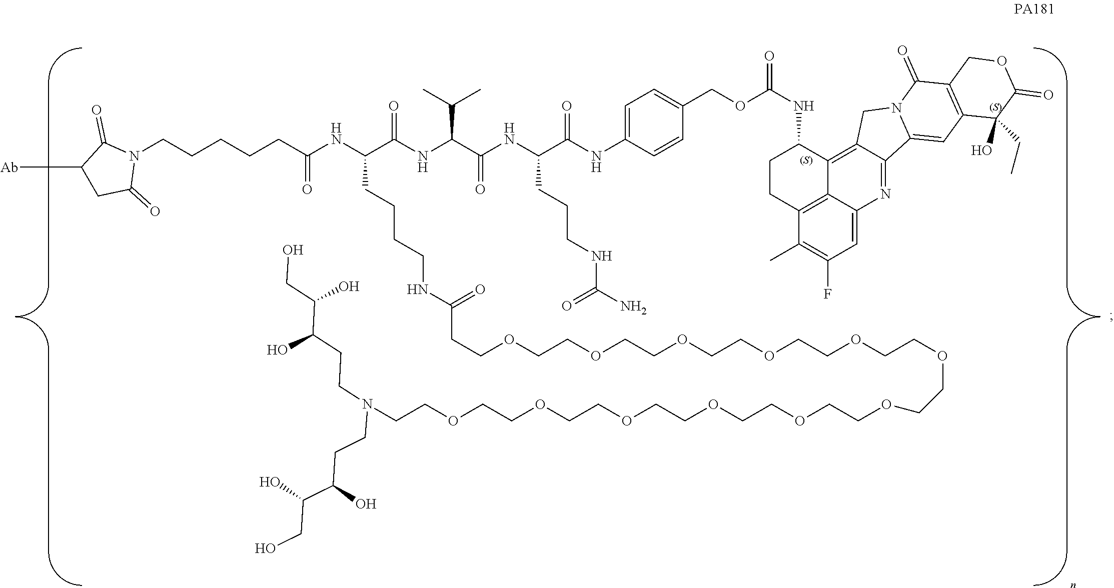 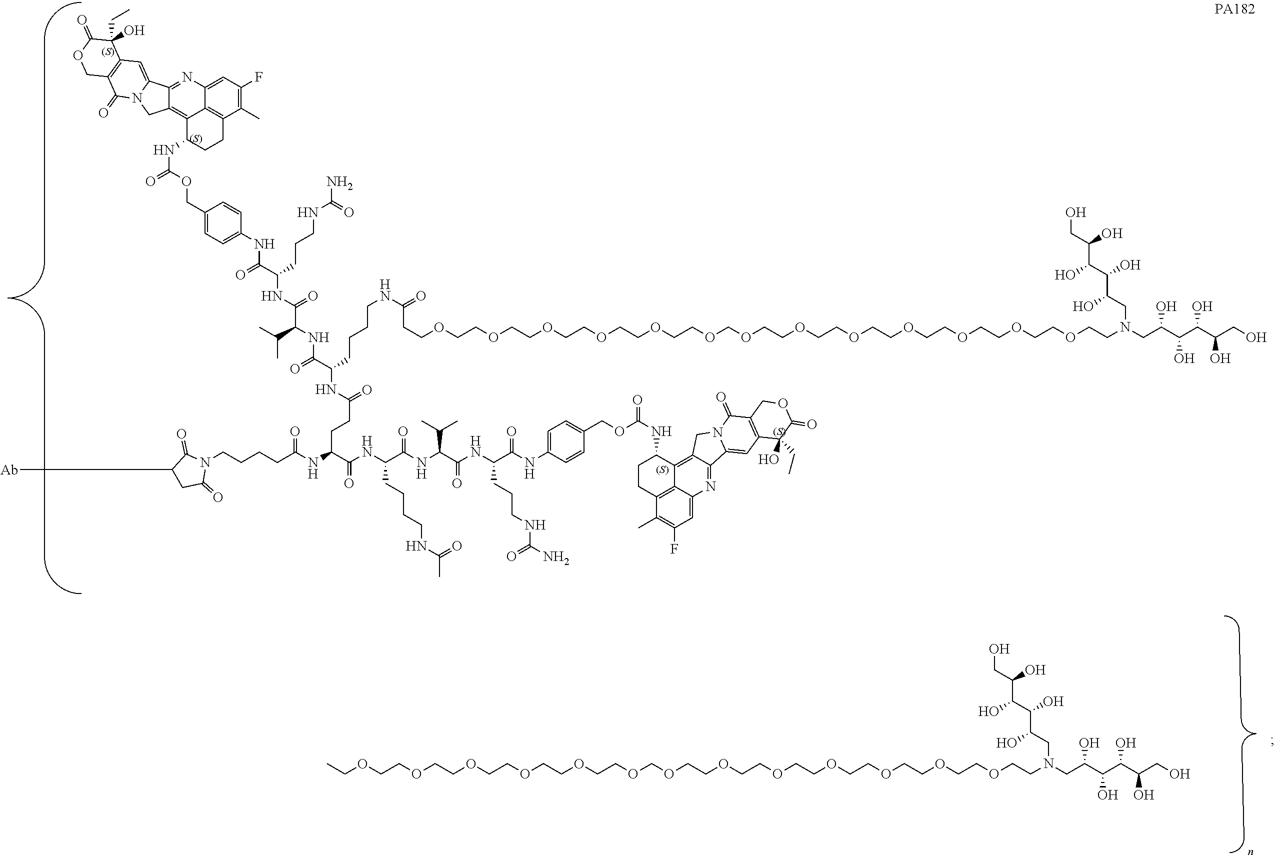 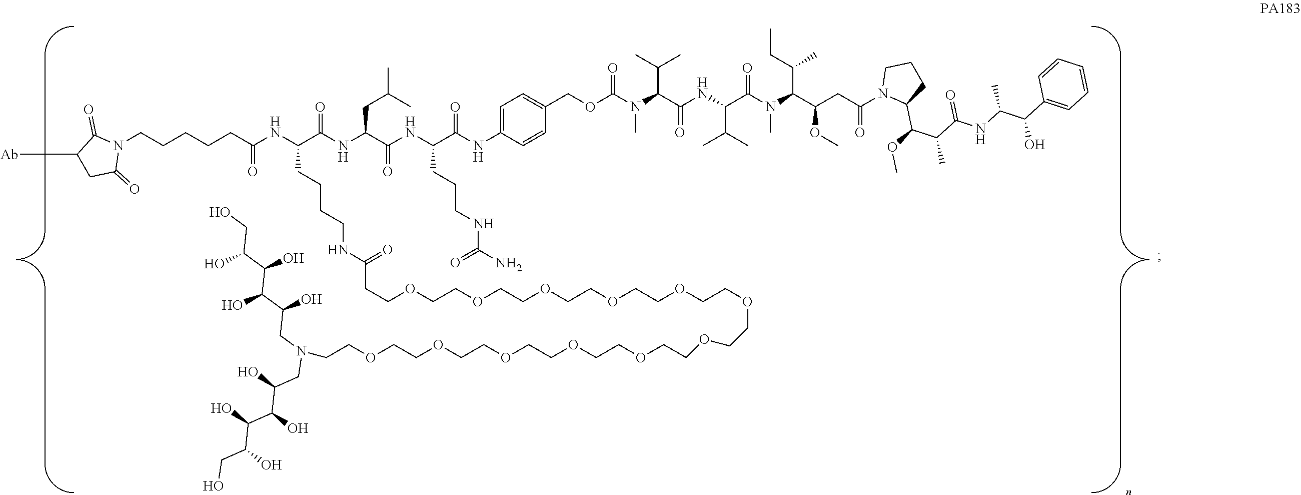 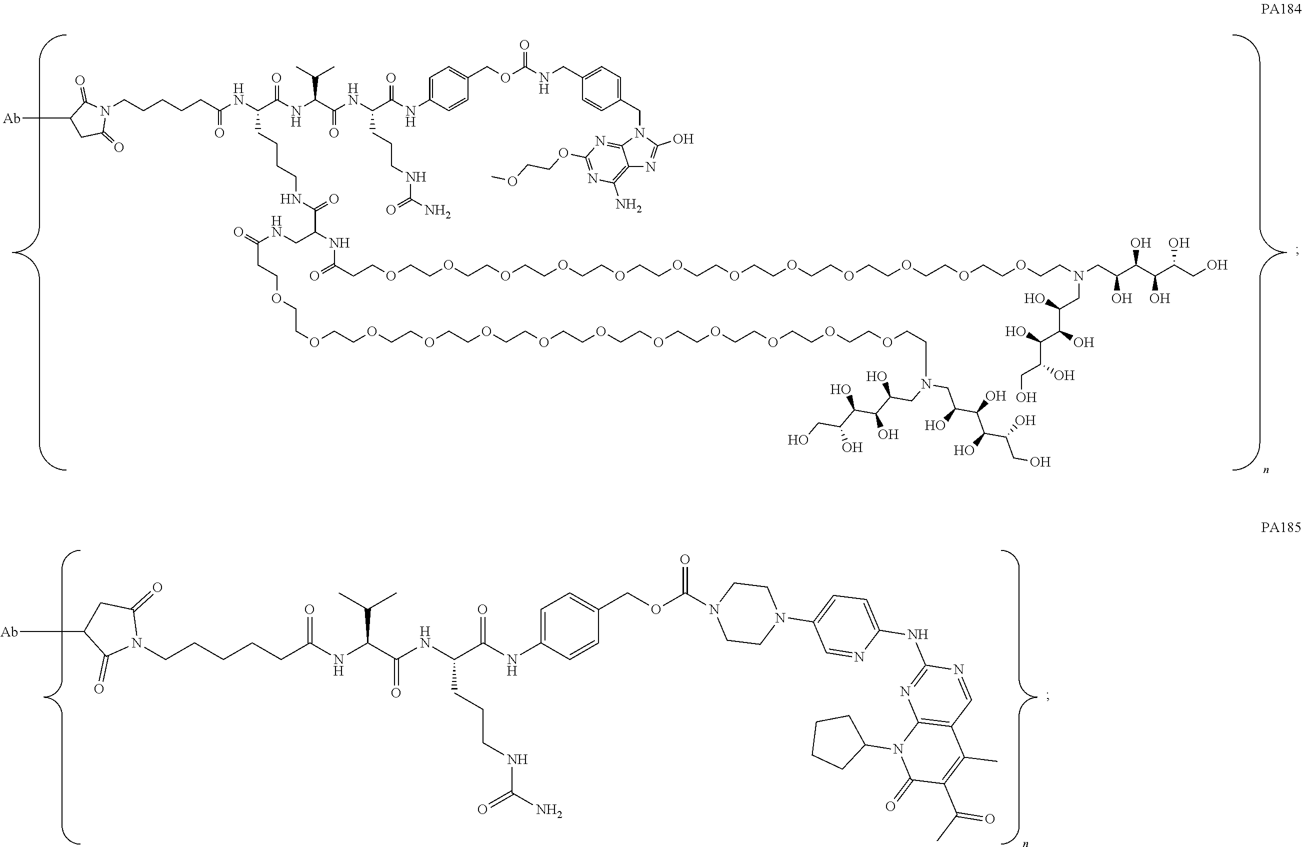 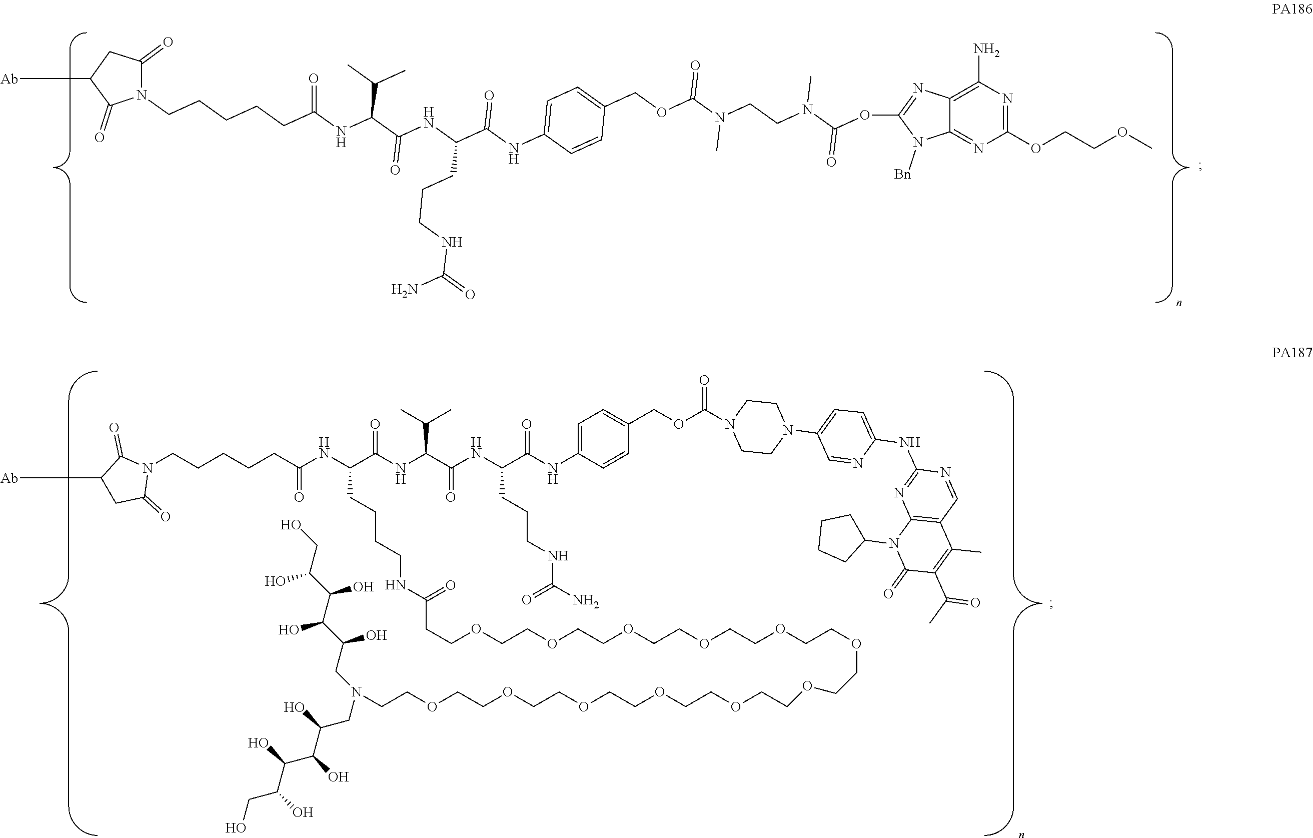 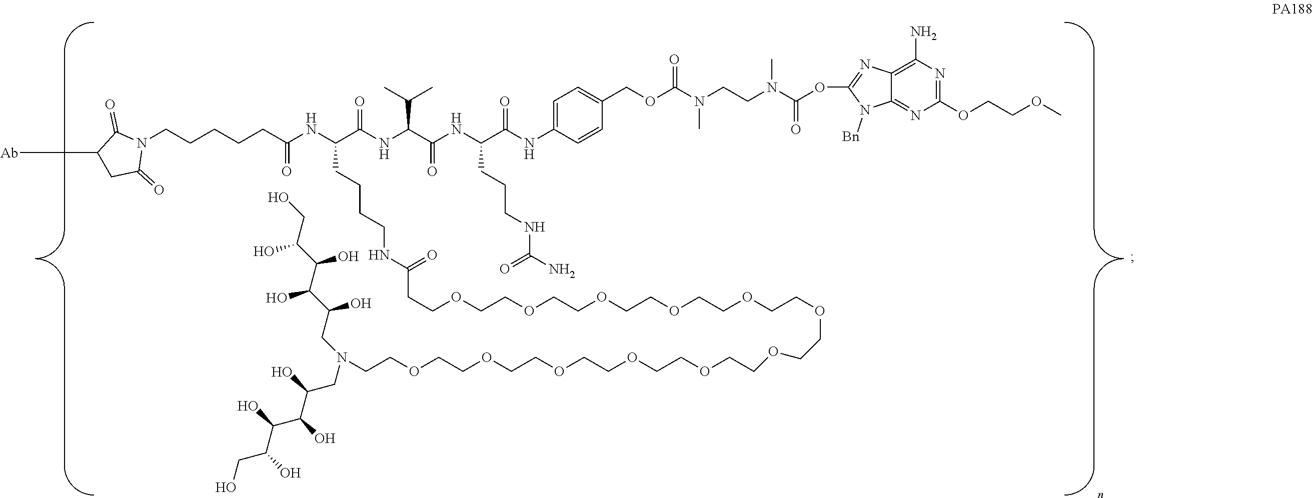 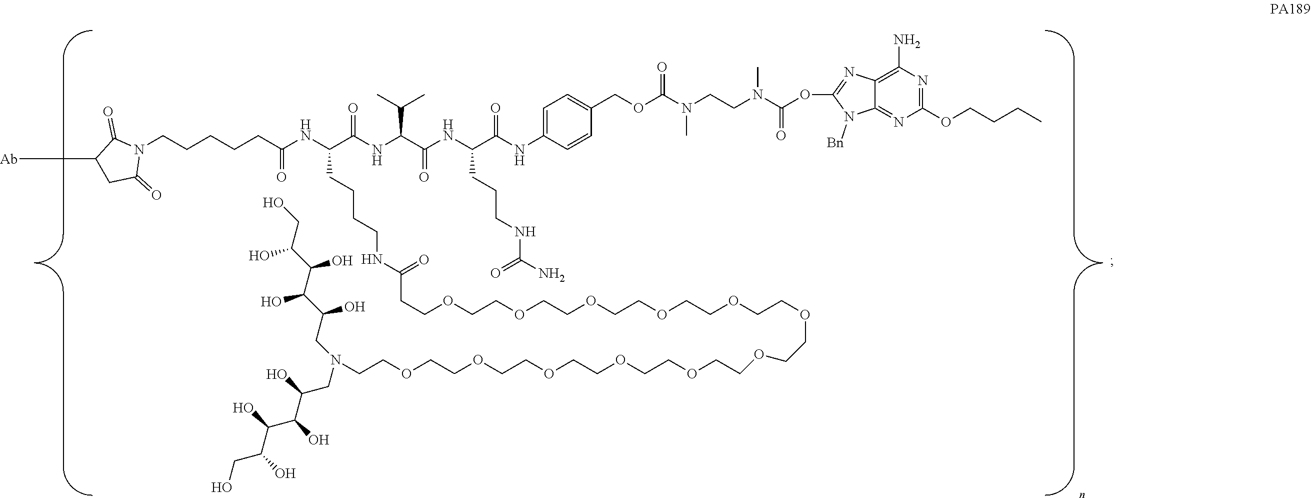 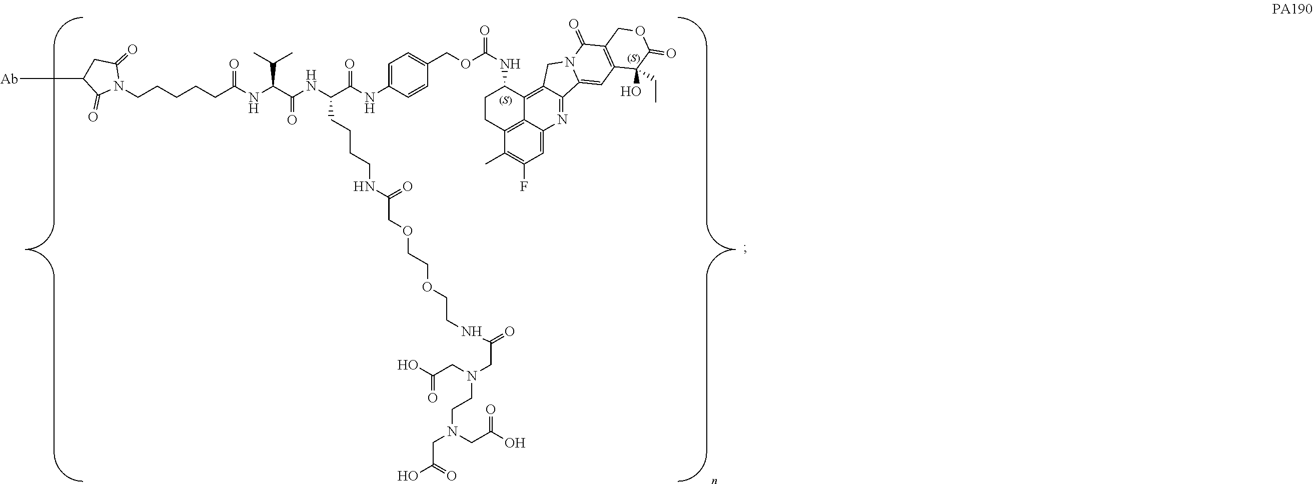 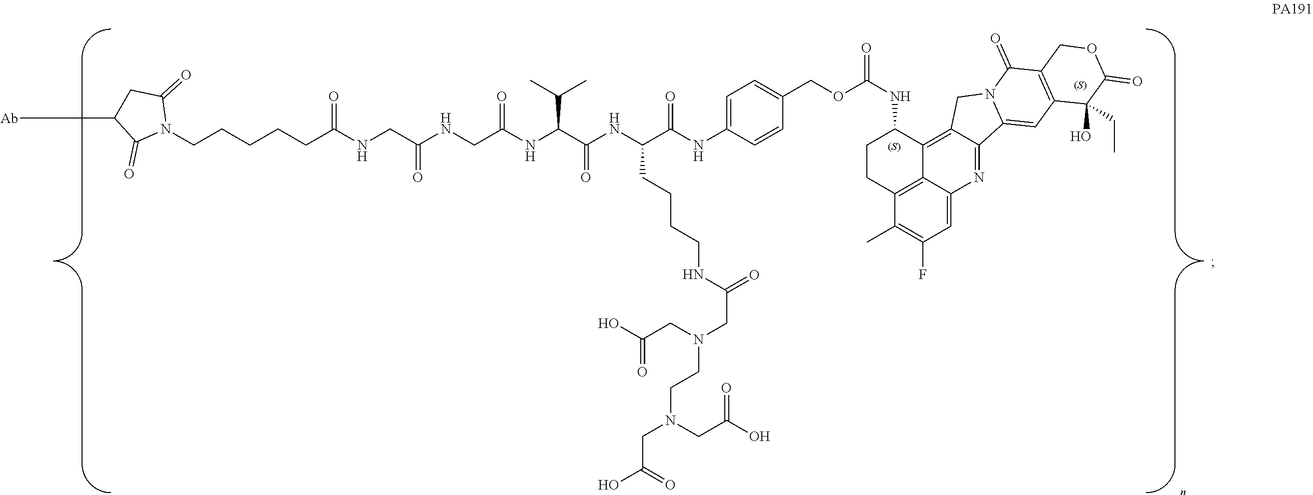 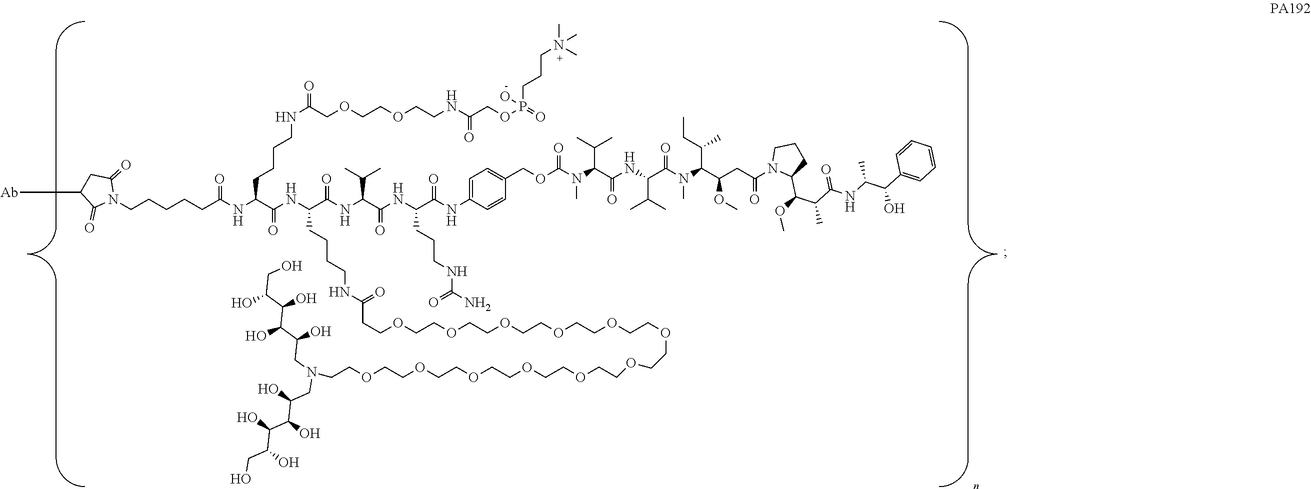 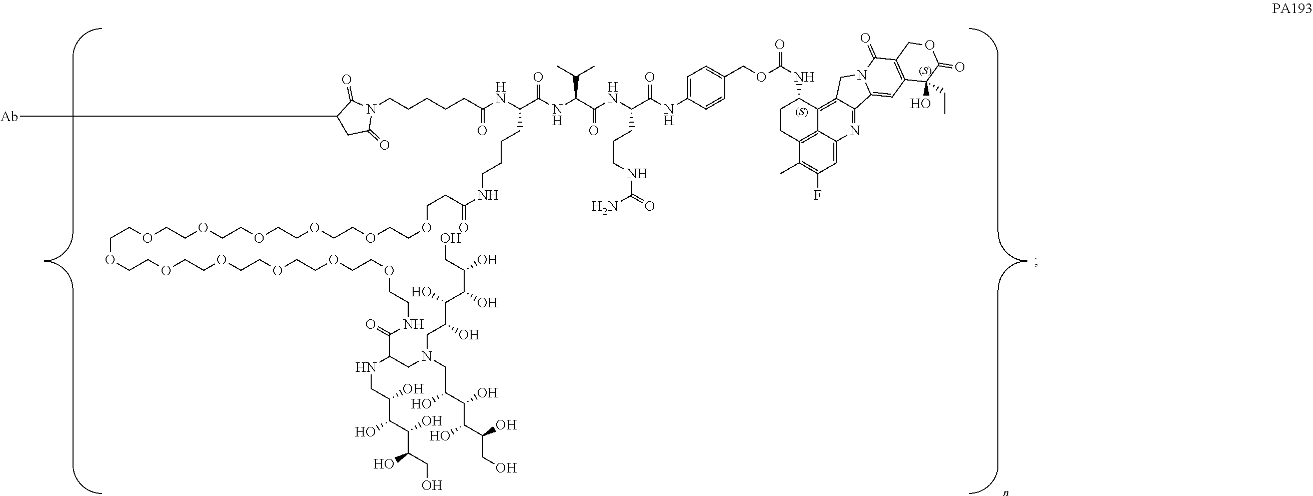 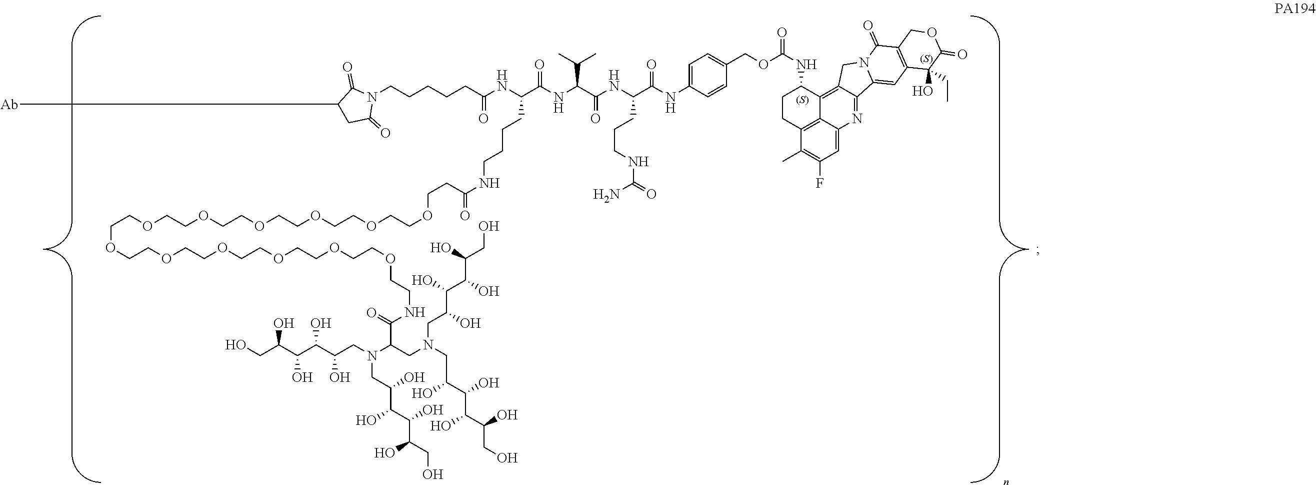 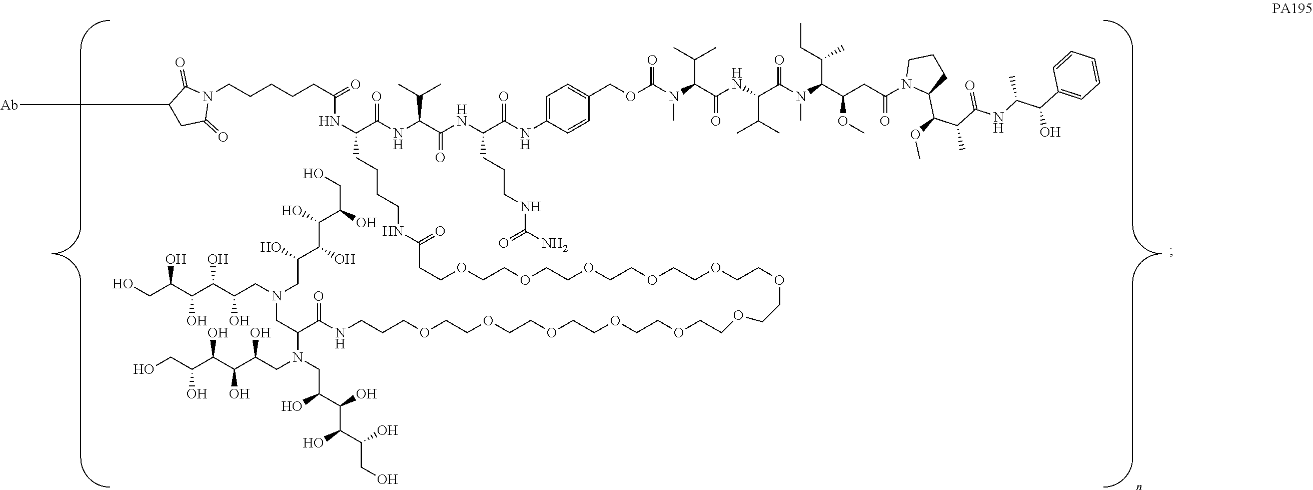 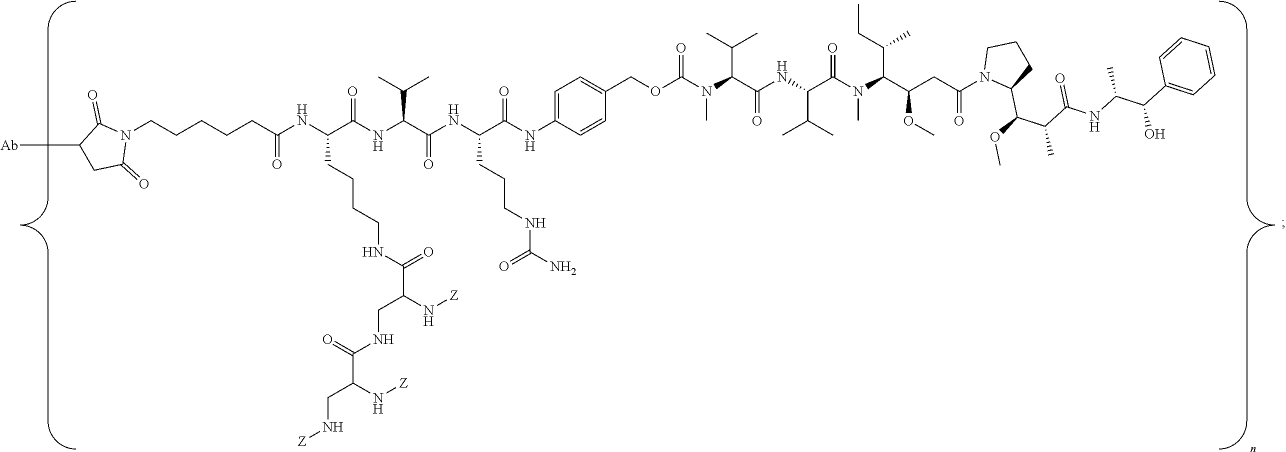
wherein each Z is attached at * and is individually selected from: 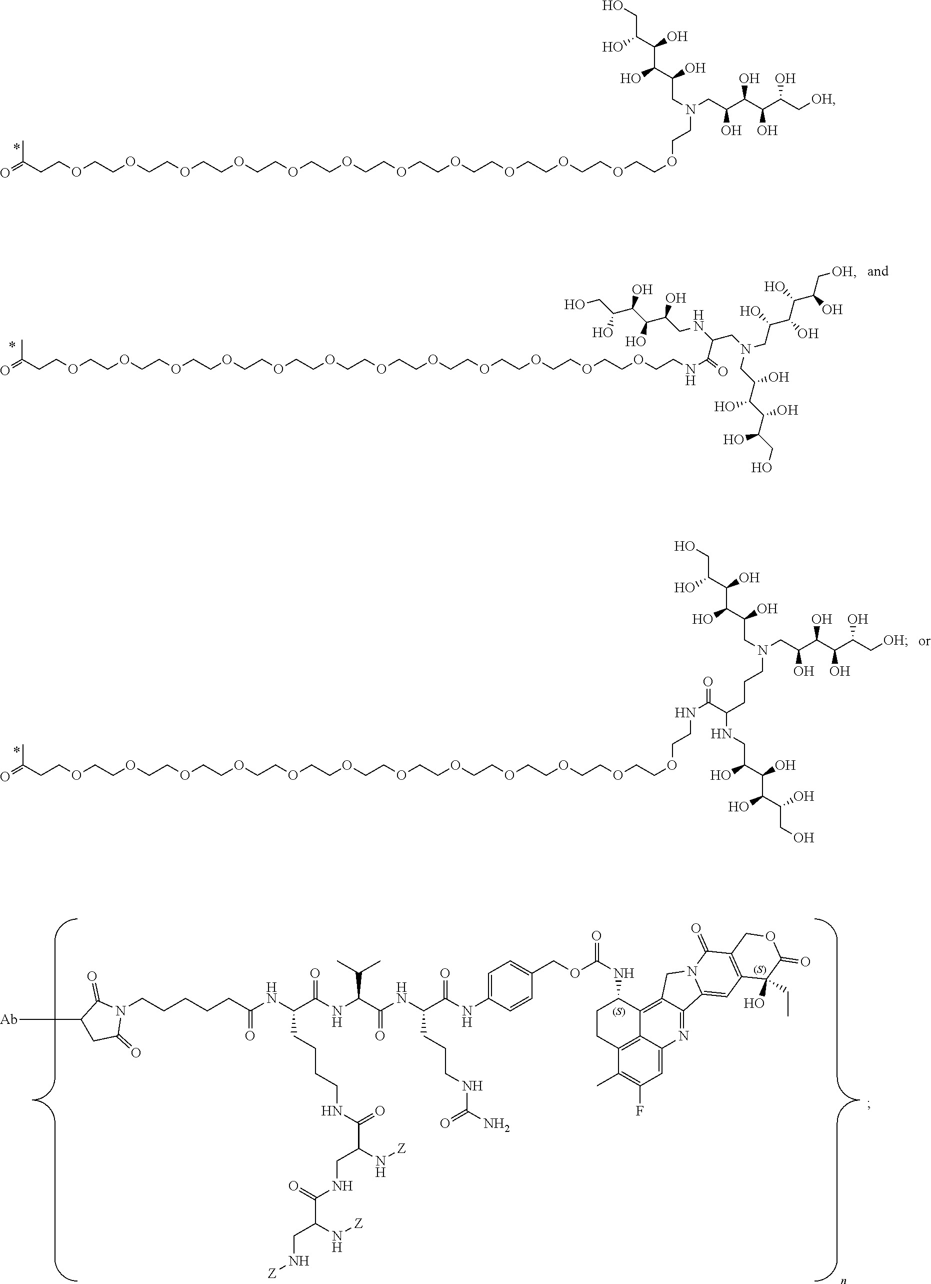
wherein each Z is attached at * and is individually selected from: 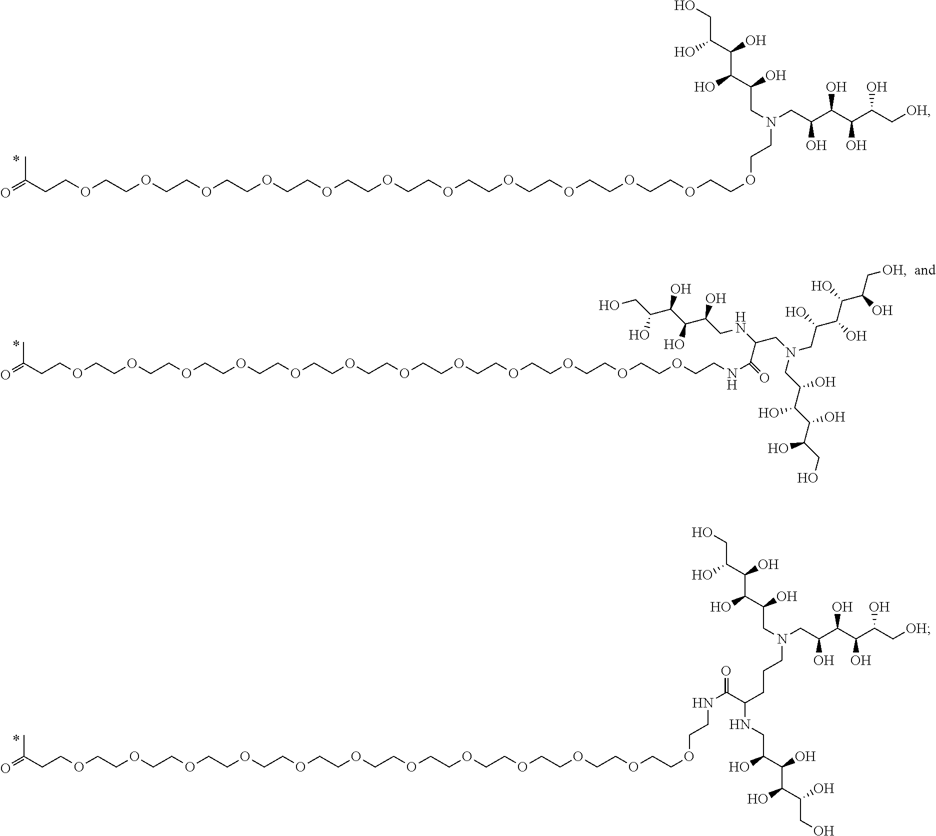
or a stereoisomer thereof, wherein Ab represents the Binding unit and n is p^{load}, wherein pload is from about 1 to about 8, about 2, about 4, about 6, about 8, about 10, about 12, about 14, about 16, about 3 to about 5, about 6 to about 8, or about 8 to about 16.

119. The conjugate of claim 3, wherein the conjugate has the following structure: 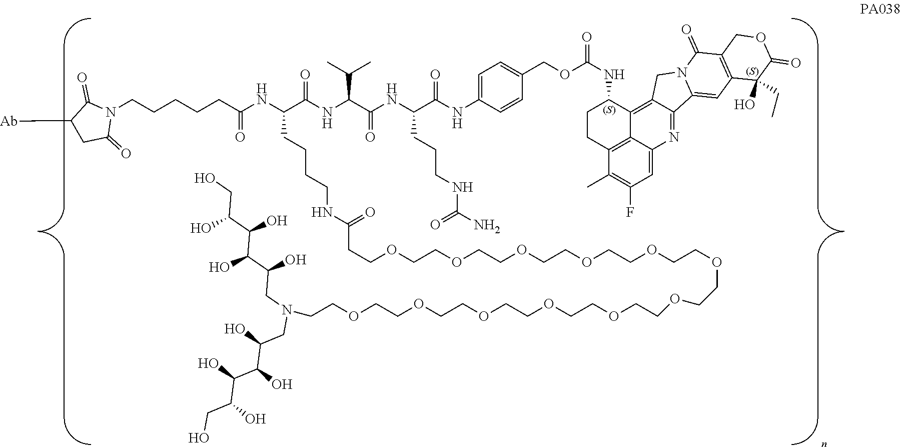
and wherein Ab is 2E7 and n is p^{load}, wherein pload is from about 1 to about 8, about 2, about 4, about 6, about 8, about 10, about 12, about 14, about 16, about 3 to about 5, about 6 to about 8, or about 8 to about 16.

120. The conjugate of claim 3, wherein the conjugate has the following structure: 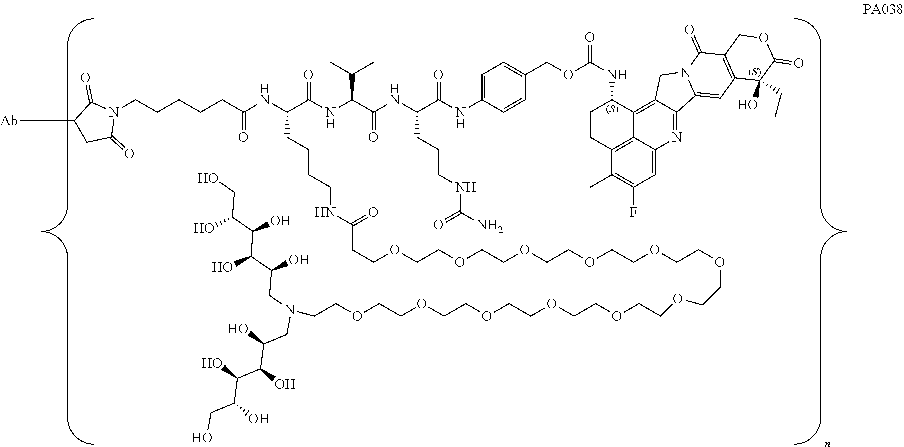
and wherein Ab is 2E7, which comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs comprising amino acids sequences selected from the sets of amino acid sequences set forth in SEQ ID NO:21, SEQ ID NO:22, SEQ ID NO:15, SEQ ID NO:24, SEQ ID NO:25 and SEQ ID NO:26, respectively,
wherein the VH and VL regions comprise amino acid sequences that are selected from the pairs of amino acid sequences set forth in SEQ ID NO:7 and SEQ ID NO:8, respectively,
wherein the binding agent further comprises a heavy chain constant region set forth in SEQ ID NO:28,
wherein the binding agent further comprises a light chain constant region set forth in SEQ ID NO:29,
wherein the binding agent is a monoclonal antibody,
and n is p^{load} and is 8.

121. A pharmaceutical composition comprising the conjugate of claim 3 and a pharmaceutically acceptable carrier.

122. A method of treating a CD70+ cancer, comprising administering to a subject in need thereof a therapeutically effective amount of the conjugate of claim 3 or the pharmaceutical composition of claim 121.

123-153. (canceled)


SEQUENCE LISTING (36 sequences)

SEQ ID NO: 1 - protein, 122 aa, synthetic construct
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNCFDYWGQGTLVTVSS

SEQ ID NO: 2 - protein, 107 aa, synthetic construct
EIVLTQSPATLSLSPGERATLSCRASQSVSSYLAWYQQKPGQAPRLLIFDASNRATGIPARFSGSGSGTDFTLTISSLEPEDFAVYYCQQRSNWPLTFGGGTKVEIK

SEQ ID NO: 3 - protein, 122 aa, synthetic construct
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDYGGNVFPYWGQGTLVTVSS

SEQ ID NO: 4 - protein, 107 aa, synthetic construct; identical to SEQ ID NO: 2

SEQ ID NO: 5 - protein, 122 aa, synthetic construct
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDFMGVCFDYWGQGTLVTVSS

SEQ ID NO: 6 - protein, 107 aa, synthetic construct; identical to SEQ ID NO: 2

SEQ ID NO: 7 - protein, 122 aa, synthetic construct
QVQLQESGPGLVKPSETLSLTCTVSGGSVSSDYYYWSWIRQPPGKGLEWLGYIYYSGSTNYNPSLKSRVTISVDTSKNQFSLKLRSVTTADTAVYYCARGDGDFLGVCFDYWGQGTLVTVSS

SEQ ID NO: 8 - protein, 107 aa, synthetic construct; identical to SEQ ID NO: 2

SEQ ID NO: 9 - protein, 122 aa, synthetic construct; identical to SEQ ID NO: 1

SEQ ID NO: 10 - protein, 107 aa, synthetic construct
EIVLTQSPATLSLSPGERATLSCRASQSVSSYLAWYQQKPGQAPRLLIFDASNRATGIPARFSGSGSGTDFTLTISSLEPEDFAVYYCQQRLKFPLTFGGGTKVEIK

SEQ ID NO: 11 - protein, 122 aa, unidentified
QVQLQESGPGLVKPSETLSLTCTVSGGSVYSGYYYWSWIRQPPGKGLEWLGYFSLSGSTNYNPSLKSRVTISVDTSKNQFSLKLRSVTTADTAVYYCARGDGDYGGNCFDYWGQGTLVTVSS

SEQ ID NO: 12 - protein, 107 aa, synthetic construct; identical to SEQ ID NO: 2

SEQ ID NO: 13 - protein, 12 aa, synthetic construct
GDGDYGGNVFPY

SEQ ID NO: 14 - protein, 12 aa, synthetic construct
GDGDFMGVCFDY

SEQ ID NO: 15 - protein, 12 aa, synthetic construct
GDGDFLGVCFDY

SEQ ID NO: 16 - protein, 8 aa, synthetic construct
YSGYYYWS

SEQ ID NO: 17 - protein, 16 aa, synthetic construct
YFSLSGSTNYNPSLKS

SEQ ID NO: 18 - protein, 9 aa, synthetic construct
QQRLKFPLT

SEQ ID NO: 19 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 20 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 21 - protein, 8 aa, synthetic construct
SSDYYYWS

SEQ ID NO: 22 - protein, 16 aa, synthetic construct
YIYYSGSTNYNPSLKS

SEQ ID NO: 23 - protein, 12 aa, synthetic construct
GDGDYGGNCFDY

SEQ ID NO: 24 - protein, 11 aa, synthetic construct
RASQSVSSYLA

SEQ ID NO: 25 - protein, 7 aa, synthetic construct
DASNRAT

SEQ ID NO: 26 - protein, 9 aa, synthetic construct
QQRSNWPLT

SEQ ID NO: 27 - protein, 5 aa, synthetic construct
GGGGS

SEQ ID NO: 28 - protein, 330 aa, synthetic construct [sequence omitted]

SEQ ID NO: 29 - protein, 107 aa, synthetic construct [sequence omitted]

SEQ ID NO: 30 - protein, 6 aa, synthetic construct
HHHHHH

SEQ ID NO: 31 - protein, 9 aa, synthetic construct
GCCGCCACC

SEQ ID NO: 32 - protein, 19 aa, synthetic construct
MGWSCIILFLVATATGVHS

SEQ ID NO: 33 - protein, 5 aa, synthetic construct
LPXTG

SEQ ID NO: 34 - protein, 4 aa, synthetic construct
  BINDING 1: note = (Succinimid-3-yl-N)-(CH2)n-C(=O)-
  BINDING 4: note = -NH-CH2-O-CH2-(C=O)-
GGFG

SEQ ID NO: 35 - protein, 4 aa, synthetic construct
GGFG

SEQ ID NO: 36 - protein, 4 aa, synthetic construct
ALAL